(19) United States
(12) Patent Application Publication
(10) Pub. No.: US 2026/0355368 A1
(43) Pub. Date: Nov. 12, 2026

(54) CONJUGATES AND USES THEREOF

(71) Applicant: Full-Life Technologies UK Limited, London (GB)

(72) Inventors: Fa LIU, Lincolnshire, IL (US); Xiaozhu WANG, Shanghai (CN)

(21) Appl. No.: 19/486,193

(22) PCT Filed: May 31, 2024
(86) PCT No.: PCT/CN2024/096705

§ 371 Date: Nov. 20, 2025

(30) Foreign Application Priority Data
May 31, 2023 (WO) PCT/CN2023/097421
Dec. 26, 2023 (WO) PCT/CN2023/141916

(51) Int. Cl. A61K 51/10; A61P 35/00

(52) U.S. Cl. CPC A61K 51/1096; A61K 51/1045; A61P 35/00

(57) ABSTRACT

Provided herein is a conjugate comprising a branching group which is at least trivalent, an antibody or antibody mimetic specifically binding to a tumor antigen expressed on a tumor cell, an effector moiety, and a potency enhancing group.

### RELATED APPLICATIONS

The present application claims the benefit of priorities of application No. PCT/CN2023/097421 filed on May 31, 2023 and PCT/CN2023/141916 filed on Dec. 26, 2023, the contents of which are incorporated herein by reference in their entirety.

### FIELD

The present application relates to a compound which can be used to conjugate with a target binding moiety to deliver an effector moiety of the compound to a target cell. Also provided is a conjugate comprising the compound and the target binding moiety and methods of preparing and using the same.

### BACKGROUND

Antibody-drug conjugate (ADC) is one of the most promising therapeutic modalities for cancer treatment. Working as a targeted therapy, ADC utilize an antibody as a targeting component to selectively deliver a cytotoxic drug to a target, such as a tumor-associated antigen. ADC therapy has achieved significant clinical and commercial successes. There are several factors contributing to the overall efficacy and safety of an ADC therapy, including plasma pharmacokinetic (PK) profile, tumor tissue penetration ability, binding affinity to the target, uptake by cancer cells, release of the active catabolic product (the toxins) within the target cells, accumulation of the toxins within tumor. Similar to ADCs, radionuclide drug conjugates (RDCs) may also employ antibodies to deliver diagnostically or therapeutically effective radioisotope payloads to targets. The high affinity and specificity of an antibody could potentially lead to high efficacy and low toxicity of a RDC therapy therefore broaden its therapeutic window. Previous studies have demonstrated that full-length antibodies could effectively carry radioisotopes to tumor tissues.

### SUMMARY

New targeted cancer therapies are still in demand in the field and are described herein. The compounds and conjugates of the present application are designed to further optimize the efficacy and safety profiles of the therapies which include but are not limited to increasing tumor cell uptake, extending tumor residency, minimizing normal organ accumulation such as in kidney, and maintaining acceptable systemic clearance such as in blood.

In one aspect, provided herein is a compound comprising a structure of formula (I-X):

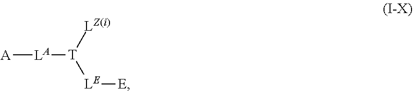

wherein:
(1) L^{Z(i)} is a linker comprising a thiol-reactive group or an amine-reactive group,
(2) T is a branching group which is at least trivalent,
(3) E is an effector moiety, wherein E is conjugated to T via a linker L^{E}, and
(4) A is a potency enhancing moiety, wherein A is conjugated to T via a linker L^{A}.

In some embodiments, A comprises one or more plasma protein binding groups. In some embodiments, the plasma protein is selected from albumin, alpha-1-acid glycoprotein, fetuin, transferrin, IgG, HDL (high-density lipoprotein) or LDL (low-density lipoprotein). In some embodiments, A comprises one or more albumin binding groups. In some embodiments, the albumin binding group is selected from the group consisting of unsubstituted or substituted C(O)C₁₋₂₆alkyleneCOOH, unsubstituted or substituted C(O)C₁₋₂₆alkenyleneCOOH, unsubstituted or substituted C(O)C₁₋₂₆alkyl, and unsubstituted or substituted C(O)C₁₋₂₆alkenyl. In some embodiments, the albumin binding group is selected from unsubstituted or substituted C(O)C₆₋₂₀alkyleneCOOH, or unsubstituted or substituted C(O)C₇₋₂₁alkyl. In some embodiments, the albumin binding group is selected from the group consisting of C(O)C₇alkyl, C(O)C₈alkyl, C(O)C₉alkyl, C(O)C₁₀alkyl, C(O)C₁₁alkyl, C(O)C₁₂alkyl, C(O)C₁₃alkyl, C(O)C₁₋₄alkyl, C(O)C₁₅alkyl, C(O)C₁₆alkyl, C(O)C₁₇alkyl, C(O)C₁₈alkyl, C(O)C₁₉alkyl, C(O)C₂₀alkyl, C(O)C₂₁alkyl, C(O)C₆alkyleneCOOH, C(O)C₇alkyleneCOOH, C(O)C₈alkyleneCOOH, C(O)C₉alkyleneCOOH, C(O)C₁₀alkyleneCOOH, C(O)C₁₁alkyleneCOOH, C(O)C₁₂alkyleneCOOH, C(O)C₁₃alkyleneCOOH, C(O)C₁₋₄alkyleneCOOH, C(O)C₁₅alkyleneCOOH, C(O)C₁₆alkyleneCOOH, C(O)C₁₇alkyleneCOOH, C(O)C₁₈alkyleneCOOH, C(O)C₁₉alkyleneCOOH, and C(O)C₂₀alkyleneCOOH. In some embodiments, the albumin binding group is C(O)C₁₋₄alkyleneCOOH, C(O)C₁₆alkyleneCOOH, or C(O)C₁₈alkyleneCOOH. In some embodiments, the albumin binding group is selected from CH₃—(CH₂)_{M1}—CO— or COOH—(CH₂)_{N1}—CO—, wherein each of M1 and N1 is independently selected from an integer of 6-22. In some embodiments, the albumin binding group is COOH—(CH₂)_{N1}—CO—, and wherein N1 is an integer of 10-18. In some embodiments, the albumin binding group is COOH—(CH₂)₁₆—CO— or COOH—(CH₂)₁₈—CO.

In some embodiments, A comprises a structure of formula (I-Dual-A):

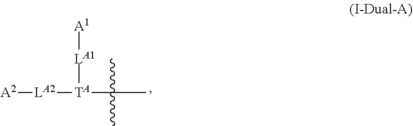

wherein each of A¹ and A² is independently a potency enhancing moiety, A¹ is conjugated to T^{A} via a linker L^{A1}, A² is conjugated to T via a linker L^{A2}, T^{A} is conjugated to T via the linker L^{A}.

In some embodiments, A¹ and A² are identical. In some embodiments, each of A¹ and A² is independently an albumin binding group. In some embodiments, each of A¹ and A² is independently selected from the group consisting of unsubstituted or substituted C(O)C₁₋₂₆alkyleneCOOH, unsubstituted or substituted C(O)C₁₋₂₆alkenyleneCOOH, unsubstituted or substituted C(O)C₁₋₂₆alkyl, and unsubstituted or substituted C(O)C₁₋₂₆alkenyl. In some embodiments, each of A¹ and A² is independently selected from unsubstituted or substituted C(O)C₆₋₂₀alkyleneCOOH, or unsubstituted or substituted C(O)C₇₋₂₁alkyl. In some embodiments, each of A¹ and A² is independently selected from the group consisting of C(O)C₇alkyl, C(O)C₈alkyl, C(O)C₉alkyl, C(O)C₁₀alkyl, C(O)C₁₁alkyl, C(O)C₁₂alkyl, C(O)C₁₃alkyl, C(O)C₁₋₄alkyl, C(O)C₁₅alkyl, C(O)C₁₆alkyl, C(O)C₁₇alkyl, C(O)C₁₈alkyl, C(O)C₁₉alkyl, C(O)C₂₀alkyl, C(O)C₂₁alkyl, C(O)C₆alkyleneCOOH, C(O)C₇alkyleneCOOH, C(O)C₈alkyleneCOOH, C(O)C₉alkyleneCOOH, C(O)C₁₀alkyleneCOOH, C(O)C₁₁alkyleneCOOH, C(O)C₁₂alkyleneCOOH, C(O)C₁₃alkyleneCOOH, C(O)C₁₋₄alkyleneCOOH, C(O)C₁₅alkyleneCOOH, C(O)C₁₆alkyleneCOOH, C(O)C₁₇alkyleneCOOH, C(O)C₁₈alkyleneCOOH, C(O)C₁₉alkyleneCOOH, and C(O)C₂₀alkyleneCOOH. In some embodiments, each of A¹ and A² is independently selected from CH₃—(CH₂)_{M1}—CO— or COOH—(CH₂)_{N1}—CO—, wherein each of M1 and N1 is independently an integer of 6-22. In some embodiments, each of A¹ and A² is independently COOH—(CH₂)_{N1}—CO—, wherein N1 is an integer of 6-20. In some embodiments, A¹ is COOH—(CH₂)₁₆—CO— and A² is COOH—(CH₂)_{N1}—CO—, wherein N1 is an integer of 8-16. In some embodiments, A¹ is COOH—(CH₂)_{N1}—CO— and A² is CH₃—(CH₂)_{M1}—CO—, wherein each of M1 and N1 is independently an integer of 10-18. In some embodiments, A¹ is COOH—(CH₂)₁₆—CO— and A² is CH₃—(CH₂)_{M1}—CO—, wherein M1 is an integer of 8-16.

In some embodiments, E comprises a chemotherapeutic agent, a toxin, an immunomodulator, a diagnostic agent, a radionuclide, or a chelating group. In some embodiments, E comprises a radionuclide selected from the group consisting of ¹¹C, ¹³N, ¹⁴C, O, ¹⁸F, ⁷⁵Br, ⁷⁶Br, ⁷⁷Br, ¹²³I, ¹²⁴I, ¹²⁵I, ¹³¹I, ³⁵S ²¹¹At, ³²P, ³³P, ⁷¹As, ⁷²As, ⁷⁴As, ⁷⁶As, and ⁷⁷As. In some embodiments, E comprises a chelating group derived from a chelating agent. In some embodiments, the chelating agent is selected from the group consisting of 1,4,7-Triazacyclononane (TACN), 1,4,7-triazacyclononane-triacetic acid (NOTA), 1,4,7-triazacyclononane-N-succinic acid-N′,N″-diacetic acid (NOTASA), 1,4,7-triazacyclononane-N-glutamic acid-N′,N″-diacetic acid (NODAGA), 1,4,7-triazacyclononane-N,N′,N″-tris(methylenephosphonic) acid (NOTP), 1,4,7,10-tetraazacyclododecane ([12]aneN4) (cyclen), 1,4,7,10-tetraazacyclotridecane ([13]aneN4), 1,4,7,11-tetraazacyclotetradecane (iso-cyclam), 1,4,7,10-tetraazacyclododecane-1,4,7,10-tetraacetic acid (DOTA), 2-(1,4,7,10-tetraazacyclododecan-1-yl)acetate (DO1A), 2,2′-(1,4,7,10-tetraazacyclododecane-1,7-diyl)diacetic acid (DO2A), 2,2′,2″-(1,4,7,10-tetraazacyclododecane-1,4,7-triyl)triacetic acid (DO3A), 1,4,7,10-tetraazacyclododecane-1,4,7,10-tetra(methanepnosphonic acid) (DOTP), 1,4,7,10-tetraazacyclododecane-1,7-di(methanephosphoriic acid) (DO2P), 1,4,7,10-tetraazacyclododecane-1,4,7-tri(methanephosphonic acid) (DO3P), 1,4,7,10-tetraazacyclo-decane-1-glutamic acid-4,7,10-triacetic acid (DOTAGA), 1,4,7,10-tetraazacyclodecane-1-succinic acid-4,7,10-triacetic acid (DOTASA), 1,4,8,11-tetraazacyclotetradecane ([14]aneN4) (cyclam), 1,4,8,12-tetraazacyclopentadecane ([15]aneN4), 1,5,9,13-tetraazacyclohexadecane ([16]aneN4), 1,4-ethano-1,4,8,11-tetraazacyclo-tetradecane (et-cyclam), 1,4,8,11-tetraazacyclotetradecane-1,4,8,1 1-tetraacetic acid (TETA), 2-(1,4,8,11-tetraazacyclotetradecane-1-y I) acetic acid (TE1A), 2,2′-(1,4,8,11-tetraazacyclotetradecane-1,8-diyl)diacetic acid (TE2A), 4,11-bis(carboxy methyl)-1,4,8,11-tetraazabicyclo[6.6.2]-hexadecane (CB-TE2A), 3,6,10,13,16,19-hexaazabicyclo[6.6.6]icosane (Sar), 1,4,7,10-tetra-(2-carbamoyl-methyl)-cyclododecane (TCMC), N,N′-bis[(6-carboxy-2-pyridil)methyl]-4,13-diaza-18-crown-6 (macropa), phthalocyanines, porphyrins, PCTA (3,6,9,15-tetraazabicyclo[9.3.1]pentadeca-1(15),11,13-triene-3,6,9-triacetic acid), DEPA (7-[2-(biscarboxymethylamino)ethyl]-4,10-biscarboxymethyl-1,4,7,10-tetraazacyclododec-1-yl-acetic acid), DTPA (1,1,4,7,7-diethylenetriaminepentaacetic acid), CHX-DTPA (cyclohexane-1,2-diamineN,N,N′,N′-tetraacetate), BATPA (1,2-bis[2-aminophenoxy]ethane-N,N,N′,N′-tetraacetic acid), TTHA (triethylenetetramineN,N,N′,N″,N″′,N″′-hexaacetic acid), HBED (N,N′-bis[2-hydroxybenzyl]ethylenediamine-N,N′-diacetic acid), EGTA (ethylene glycol bis[2-aminoethyl ether]-N,N,N′,N′-tetraacetic acid), EDTMP (ethylenediamine tetra-[methylene phosphonic acid]), TRAP (triazacyclononate phosphinic acids), SHBED (N,N′-bis[2-hydroxy-5-sulfobenzyl]ethylenediaminediacetic acid), H6Sbbpen(N,N′-bis-[2-hydroxy-5-sulfonylbenzyl]-N,N′-bis[2-methylpyridyl]ethylenediamine), THP (Tris(3,4-hydroxypyridinone), DFO (deferoxamine), FSC (Fusarinine), TAFC (triacetylfusarinine C), FOXE (ferrioxamine E), 6SS (N,N′-bis[2,2-dimethyl-2-mercaptoethyl]ethylenediamine-N,N′-diacetic acid), ECC (ethylenecysteamine cysteine), ECD (ethyl cysteinate dimer), NETA ([2-{4,7-biscarboxymethyl(1,4,7)triazacyclonona-1-yl-ethyl}carbonylmethylamino]acetic acid, THPN (Tetrakis(3-Hydroxy-4-Pyridinone)), H2dedpa (1,2-[{6-(carboxylato-)pyridin-2-yl}methylamino]-ethane), H4octapa (N,N′-bis[6-carboxy-2-pyridylmethyl]-ethylenediamine-N,N′-diacetic acid), H2bispa2 (6,6′-[{9-hydroxy-1,5-bis-(methoxycarbonyl)-2,4-di(pyridin-2-yl)-3,7-diazabicyclo[3.3.1]nonane-3,7-diyl}bis(methylene)]dipicolinic acid), DOTMP(1,4,7,10-Tetraazacyclododecane-1,4,7,10-tetrayl-tetrakis(methylphosphonic acid)), PEPA (1,4,7,10,13-pentaazocyclopentadecane pentaacetic acid), HEHA (1,4,7,10,13,16-hexaazocyclooctadecane hexaacetic acid), H2hox, H2CHXhox, H2octox, H2pyhox, H4neunopa, TETPA, H4pypa, H4py4pa, DTPAm, EGTAm, ampam, Me-3,2-HOPO, 3,4,3-(L1-1,2-HOPO), and macrocyclic tetrapthalimide. In some embodiments, the chelating group is derived from DOTA or DOTAGA. In some embodiments, each of L^{E} and L^{A} is independently selected from a direct bond or a divalent linker. In some embodiments, the divalent linker comprises one or more groups selected from the group consisting of R¹NC₁₋₂O alkyleneNR², R¹NC₁₋₂₀alkenyleneNR², C(O)C₁₋₂₀alkyleneC(O), C(O)C₁₋₂₀alkenyleneC(O), R¹NC₁₋₂₀alkyleneC(O), R¹NC₁₋₂₀alkenyleneC(O), C(O)C₁₋₂O alkyleneNHR², C(O)C₁₋₂₀alkenyleneNR², C(S)C₁₋₂₀alkyleneC(S), C(S)C₁₋₂₀alkenyleneC(S), C(S)C₁₋₂₀alkyleneC(O), C(S)C₁₋₂₀alkenyleneC(O), C(O)C₁₋₂₀alkyleneC(S), C(O)C₁₋₂₀alkenyleneC(S), C(O)C₁₋₂₀alkyleneO, C(O)C₁₋₂₀alkenyleneO, R¹NC₁₋₂₀alkenyleneC(S), C(S)C₁₋₂₀alkyleneNR² and C(S)C₁₋₂₀alkenyleneNR², each of which is independently and optionally interrupted by one or more of S, O, NH, N(C₁₋₆alkyl), C(O), C(O)NH, NHC(O), C(S)NH, NHC(S), NHC(O)NH, NHC(S)NH, NHC(NH), NHC(NC₁₋₄alkyl), C(NH)NH, C(NC₁₋₄alkyl)NH, NC₄₋₁₀cycloalkyl, C₄₋₁₀heterocycloalkyl, C₆₋₁₀aryl and C₅₋₁₀heteroaryl, and wherein each alkyl, alkylene and alkenylene is optionally substituted with one or more substituents selected from halo, COOH, C₁₋₆alkyl, OH, OC₁₋₆alkyl, SH, SC₁₋₆alkyl, NR³R⁴, C₁₋₄alkyleneOH, C₁₋₄alkyleneOC₁₋₄alkyl and C₁₋₄alkyleneNR³R⁴, and each R¹, R², R³ and R⁴ is independently selected from H and C₁₋₄alkyl.

In some embodiments, the divalent linker comprises one or more amino acid residues. In some embodiments, the amino acid residue is derived from a naturally occurring amino acid selected from the group consisting of alanine (Ala, A), arginine (Arg, R), asparagine (Asn, N), aspartic acid (Asp, D), glutamine (Gln, Q), glutamic acid (Glu, E), γGlu, glutamine (Gln), glycine (Gly, G), histidine (His, H), isoleucine (Ile, I), leucine (Leu, L), lysine (Lys, K), εLys, methionine (Met, M), phenylalanine (Phe, F), proline (Pro, P), serine (Ser, S), threonine (Thr, T), tryptophan (Trp, W), tyrosine (Tyr, Y), valine (Val, V), pyrrolysine (Pyl, O), pyrroline-carboxy-lysine (PCL), gamma-carboxyglutamic acid (Gla), and a D enantiomer thereof. In some embodiments, the amino acid residue is derived from a unnaturally occurring amino acid selected from the group consisting of NR⁵C₅alkyleneC(O) (Ahx), L-Cysteic acid (Cya), norleucine (Nle), norvaline (Nva), 2-aminooctanoic acid (Aoc), 2-naphthylalanine (2-Nal), 3-(trifluoromethyl)phenylalanine (TFP), homophenylalanine (hPhe), cyclohexylalanine (Cha), 1-naphthylalanine (1-Nal), 4-benzoyl-L-phenylalanine (Bpa), 2-methoxy-4-vinylphenylalanine (MvF), 4-fluorophenylalanine (4-F-Phe), 4-phenyl-2,3-dihydroxy-6-nitrophenylalanine (pNIPA), 2-(2-naphthyl)alanine (2-Nal-ala), 4-(4-propoxyphenyl)alanine (Ppa), 4-carboxyphenylalanine (4-CPA), 4-butylphenylalanine (Bua), 2-nitrophenylalanine (2-Npa), 4-azidophenylalanine (4-AzF), 2-(4-nitrophenyl)ethylalanine (2-Npe), 3-iodo-L-tyrosine (Ity), and 5,5,5-trifluoroleucine (TFL), and a D enantiomer thereof. In some embodiments, the divalent linker comprises one of more moieties of —(OCH₂CH₂)ₙ—, wherein n is an integer of 1-28. In some embodiments, the divalent linker comprises one or more moieties each independently selected from the group consisting of —(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), —(C₂alkyleneO)₆C₂alkyleneC(O) (PEG6), —(C₂alkyleneO)₆C₂alkyleneC(O) (PEG8), and —(C₂alkyleneO)₆C₂alkyleneC(O) (PEG12).

In some embodiments, at least one of L^{E} and L^{A} is a cleavable linker. In some embodiments, the cleavable linker comprises one or more cleavable moieties selected from the group consisting of S—S, C(O)O, OC(O), OC(O)O, OC(O)NH, NHC(O)O, SC(O), C(O)S, C═NNH, C═NO, NC₁₋₄alkylC(O)O, NHOC(O)NH, NC₁₋₄alkylOC(O), and an enzymatically cleavable peptide sequence. In some embodiments, the cleavable linker comprises one or more groups selected from the group consisting of R⁵NC₁₋₂₀alkyleneNR⁶, R⁵NC₁₋₂₀alkenyleneNR⁵, C(O)C₁₋₂₀alkyleneC(O), C(O)C₁₋₂₀alkenyleneC(O), R⁵NC₁₋₂₀alkyleneC(O), R⁵NC₁₋₂₀alkenyleneC(O), C(O)C₁₋₂₀alkyleneNR⁶, C(O)C₁₋₂₀alkenyleneNR⁶, C(S)C₁₋₂₀alkyleneC(S), C(S)C₁₋₂₀alkenyleneC(S), C(S)C₁₋₂₀alkyleneC(O), C(S)C₁₋₂₀alkenyleneC(O), C(O)C₁₋₂₀alkyleneC(S), C(O)C₁₋₂₀alkenyleneC(S), SC₁₋₂₀alkyleneS, SC₁₋₂₀alkenyleneS, SC₁₋₂₀alkyleneNR⁶, SC₁₋₂₀alkenyleneNR⁶, R⁵NC₁₋₂₀alkyleneS, R⁵NC₁₋₂₀alkenyleneS, R⁵NC₁₋₂₀alkyleneO, R⁵NC₁₋₂₀alkenyleneO, OC₁₋₂O alkyleneNR⁶, OC₁₋₂₀alkenyleneNR⁶, SC₁₋₂₀alkyleneO, SC₁₋₂₀alkenyleneO, OC₁₋₂₀alkyleneS, and OC₁₋₂₀alkenyleneS, C(O)C₁₋₂₀alkyleneO, C(O)C₁₋₂₀alkenyleneO, OC₁₋₂₀alkyleneC(O), OC₁₋₂₀alkenyleneC(O), C(O)C₁₋₂₀alkyleneS, C(O)C₁₋₂₀alkenyleneS, SC₁₋₂₀alkyleneC(O), SC₁₋₂₀alkenyleneC(O), R⁵NC₁₋₂₀alkyleneC(S), R⁵NC₁₋₂₀alkenyleneC(S), C(S)C₁₋₂₀alkyleneNR⁶, C(S)C₁₋₂₀alkenyleneNR⁶, C(S)C₁₋₂₀alkyleneO, C(S)C₁₋₂₀alkenyleneO, OC₁₋₂₀alkyleneC(S), OC₁₋₂₀alkenyleneC(S), SC₁₋₂₀alkyleneC(S), SC₁₋₂₀alkenyleneC(S), OC₁₋₂₀alkyleneO, OC₁₋₂₀alkenyleneCO, SC₁₋₂₀alkyleneS, or SC₁₋₂₀alkenyleneS, each of which is independently and optionally interrupted by one or more of S—S, C(O)O, OC(O), OC(O)O, OC(O)NH, NHC(O)O, SC(O), C(O)S, NC₁₋₄alkylC(O)O, NHOC(O)NH, NC₁₋₄alkylOC(O), C═NNH, C═NNH₂, C═NOH, C═NO, NH—NH, NH—NC₁₋₄alkyl, NC₁₋₄alkyl-NH, NC₁₋₄alkylNC₁₋₄alkyl, S, O, NH, N(C₁₋₆alkyl), C(O), C(O)NH, NHC(O), NHC(O)NH, NHC(S)NH, C(S)NH, NHC(S), NHC(NH), NHC(NC₁₋₄alkyl), C(NH)NH, C(NC₁₋₄alkyl)NH, NC₄₋₁₈cycloalkyl, C₄₋₁₀heterocycloalkyl, C₆₋₁₀aryl and C₅₋₁₀heteroaryl, and wherein each alkyl, alkylene or alkenylene is optionally substituted with one or more substituents selected from halo, COOH, C₁₋₆alkyl, OH, OC₁₋₆alkyl, SH, SC₁₋₆alkyl, NR⁷R⁷, C₁₋₄alkyleneOH, C₁₋₄alkyleneOC₁₋₄alkyl and C₁₋₄alkyleneNR⁷R⁸, and each of R⁵, R⁶, R⁷ and R⁸ is independently selected from H and C₁₋₄alkyl.

In some embodiments, the cleavable linker comprises or is a structure of Formula (I-CL3):

—Y¹—W¹—Y²—W²—Y³—W³—Y⁴—W⁴—Y⁵—W⁵—Y⁶—, (I-CL3)
wherein each of W¹, W², W³, W⁴, or W⁵ is independently a bond, or unbranched or branched C₁₋₆alkylene;
wherein each of Y¹, Y², Y³, Y⁴, Y⁵, Y⁶ is independently a bond, O, S, S—S, C(O)O, OC(O), OC(O)O, OC(O)NR^{Y}, NR^{Y}C(O)O, NR^{Y}, C(O), C(O)NR^{Y}, NR^{Y}C(O), NR^{Y}C(O)NR^{Y}, C₅₋₇cycloalkyl, C₄₋₆heterocycloalkyl, or CH(RAA),
wherein each R^{Y} is independently H, C₁₋₆alkyl, C₁₋₆alkyl substituted with one or more independently selected halogen, C₁₋₆alkyleneOH, C₁₋₆alkyleneNR⁷R⁸, C₁₋₅alkyleneCOOH, or C₁₋₄alkyleneOC₁₋₄alkyl; and
wherein R^{AA} is a side chain of a naturally occurring amino acid;
provided that at least one of Y¹, Y², Y³, Y⁴, Y⁵, Y⁶ is not a bond.

In some embodiments, at least one of Y¹, Y², Y³, Y⁴, Y⁵, Y⁶ is C(O)O, OC(O), OC(O)O, OC(O)NR^{Y}, or NR^{Y}C(O)O. In some embodiments, the cleavable linker comprises one or more groups comprising a structure of Formula (I-CL1):

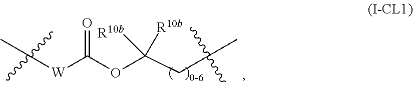

wherein W is selected from C(R^{10a})₂, N(R^{10a}) or O,
each R^{10a} is independently H, C₁₋₆alkyl, C₁₋₆alkyl substituted with one or more independently selected halogen, C₁₋₆alkyleneOH, C₁₋₆alkyleneNR⁷R⁸, C₁₋₅alkyleneCOOH, C₁₋₄alkyleneOC₁₋₄alkyl, or the side chain of a naturally occurring amino acid, or two R^{10a} groups, together with the carbon atom to which they are attached, form a C₃₋₁₀cycloalkyl or C₃₋₁₀heterocycloalkyl;
each R^{10b} is independently H, C₁₋₆alkyl, C₁₋₆alkyl substituted with one or more independently selected halogen, C₁₋₆alkyleneOH, C₁₋₆alkyleneNR⁷R⁸, C₁₋₅alkyleneCOOH, C₁₋₄alkyleneOC₁₋₄alkyl, or two R^{10b} groups, together with the carbon atom to which they are attached, form a C₃₋₁₀cycloalkyl or C₃₋₁₀heterocycloalkyl; and R⁷ and R⁸ are each independently H, or C₁₋₄alkyl.

In some embodiments, the cleavable linker comprises one or more groups selected from the group consisting of

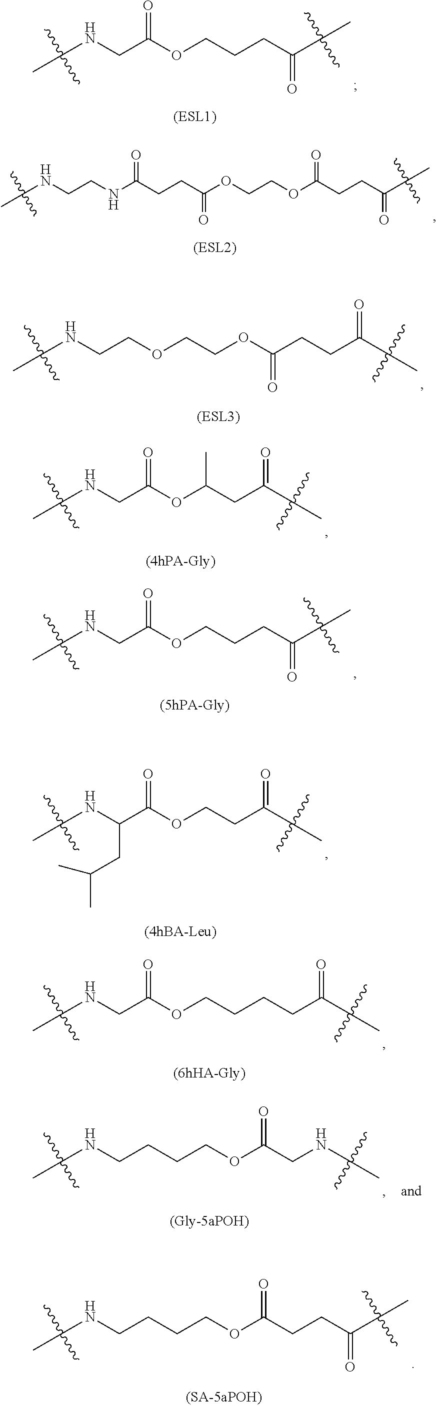

In some embodiments, at least one of Y¹, Y², Y³, Y⁴, Y⁵, Y⁶ is —S—S—. In some embodiments, the cleavable linker comprises one or more groups having a structure of Formula (I-CL2):

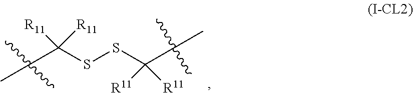

wherein each R¹¹ is independently selected from H or C₁₋₄alkyl.

In some embodiments, the cleavable linker comprises one or more groups selected from the group consisting of

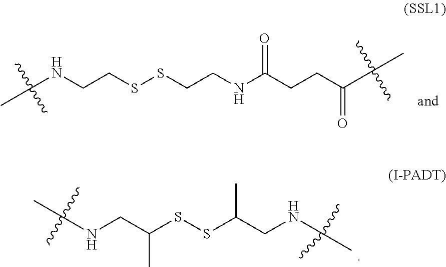 [SMILES: *NCCSSCCNC(=O)CCC(*)=O ; *NCC(C)SSC(C)CN*]

In some embodiments, the enzymatically cleavable peptide sequence is selected from the group consisting of Met-Val-Lys, Ala-Val, Ala-Pro, Gly-Pro, Ser-Ser, Ser-Gly, Gly-Ala and Ser-Lys.

In some embodiments, L^{Z(i)} comprises a thiol-reactive group. In some embodiments, the thiol-reactive group is selected from a thiol-reactive double bond, a thiol-reactive triple bond, or a leaving group containing moiety. In some embodiments, the thiol-reactive group is selected from the group consisting of

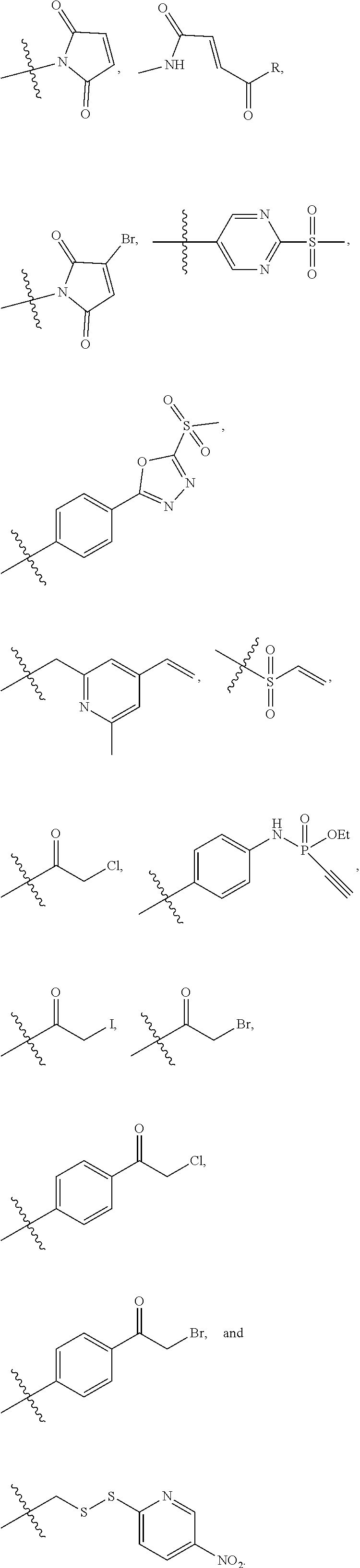

In some embodiments, L^{Z(i)} comprises an amine-reactive group. In some embodiments, the amine-reactive group is selected from the group consisting of

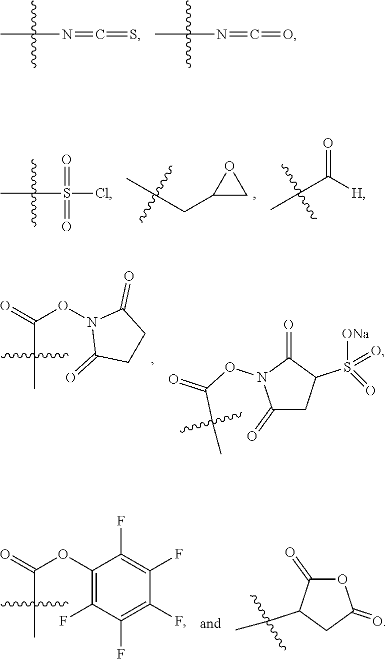

In some embodiments, L^{Z(i)} further comprises one or more groups selected from an R¹NC₁₋₂₀alkyleneNR², R¹NC₁₋₂₀alkenyleneNR² C(O)C₁₋₂₀alkyleneC(O), C(O)C₁₋₂₀alkenyleneC(O), R¹NC₁₋₂₀alkyleneC(O), R¹NC₁₋₂₀alkenyleneC(O), C(O)C₁₋₂O alkyleneNHR², C(O)C₁₋₂₀alkenyleneNR², C(S)C₁₋₂₀alkyleneC(S), C(S)C₁₋₂₀alkenyleneC(S), C(S)C₁₋₂₀alkyleneC(O), C(S)C₁₋₂₀alkenyleneC(O), C(O)C₁₋₂₀alkyleneC(S), C(O)C₁₋₂₀alkenyleneC(S), C(O)C₁₋₂₀alkyleneO, C(O)C₁₋₂₀alkenyleneO, R¹NC₁₋₂₀alkenyleneC(S), C(S)C₁₋₂₀alkyleneNR² or C(S)C₁₋₂₀alkenyleneNR², each of which is independently and optionally interrupted by one or more of S, O, NH, N(C₁₋₆alkyl), C(O), C(O)NH, NHC(O), C(S)NH, NHC(S), NHC(O)NH, NHC(S)NH, NHC(NH), NHC(NC₁₋₄alkyl), C(NH)NH, C(NC₁₋₄alkyl)NH, NC₄₋₁₀cycloalkyl, C₄₋₁₀heterocycloalkyl, C₆₋₁₀aryl and C₅₋₁₀heteroaryl, and each alkyl, alkylene and alkenylene is optionally and independently substituted with one or more substituents selected from halo, COOH, C₁₋₆alkyl, OH, OC₁₋₆alkyl, SH, SC₁₋₆alkyl, NR³R⁴, C₁₋₄alkyleneOH, C₁₋₄alkyleneOC₁₋₄alkyl and C₁₋₄alkyleneNR³R⁴, and wherein each R¹, R², R³ and R⁴ is independently selected from H and C₁₋₄alkyl.

In some embodiments, L^{Z(i)} comprises one or more amino acid residues. In some embodiments, the amino acid residue is derived from a naturally occurring amino acid selected from the group consisting of alanine (Ala, A), arginine (Arg, R), asparagine (Asn, N), aspartic acid (Asp, D), glutamine (Gln, Q), glutamic acid (Glu, E), γGlu, glutamine (Gln), glycine (Gly, G), histidine (His, H), isoleucine (Ile, I), leucine (Leu, L), Lysine (Lys, K), εLys, methionine (Met, M), phenylalanine (Phe, F), proline (Pro, P), serine (Ser, S), threonine (Thr, T), tryptophan (Trp, W), tyrosine (Tyr, Y), valine (Val, V), pyrrolysine (Pyl, O), pyrroline-carboxy-lysine (PCL), gamma-carboxyglutamic acid (Gla), and a D enantiomer thereof. In some embodiments, the amino acid residue is derived from a unnaturally occurring amino acid selected from the group consisting of NR⁵C₅alkyleneC(O) (Ahx), L-Cysteic acid (Cya), norleucine (Nle), norvaline (Nva), 2-aminooctanoic acid (Aoc), 2-naphthylalanine (2-Nal), 3-(trifluoromethyl)phenylalanine (TFP), homophenylalanine (hPhe), cyclohexylalanine (Cha), 1-naphthylalanine (1-Nal), 4-benzoyl-L-phenylalanine (Bpa), 2-methoxy-4-vinylphenylalanine (MvF), 4-fluorophenylalanine (4-F-Phe), 4-phenyl-2,3-dihydroxy-6-nitrophenylalanine (pNIPA), 2-(2-naphthyl)alanine (2-Nal-ala), 4-(4-propoxyphenyl)alanine (Ppa), 4-carboxyphenylalanine (4-CPA), 4-butylphenylalanine (Bua), 2-nitrophenylalanine (2-Npa), 4-azidophenylalanine (4-AzF), 2-(4-nitrophenyl)ethylalanine (2-Npe), 3-iodo-L-tyrosine (Ity), or 5,5,5-trifluoroleucine (TFL), and a D enantiomer thereof.

In some embodiments, L^{Z(i)} comprises one of more moieties of —(OCH₂CH₂)ₙ—, wherein n is an integer of 1-28. In some embodiments, L^{Z(i)} comprises one or more moieties each independently selected from the group consisting of —(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), —(C₂alkyleneO)₆C₂alkyleneC(O) (PEG6), —(C₂alkyleneO)₆C₂alkyleneC(O) (PEG8), and —(C₂alkyleneO)₆C₂alkyleneC(O) (PEG12).

In some embodiments, T comprises one or more amino acid residues each independently derived from Lys, ornithine (Orn), homo-lysine, 2,3-diaminopropionic acid (Dap), 2,4-diaminobutyric acid (Dab), cysteine, homo-cysteine, glutamine, glutamic acid, asparagine, aspartic acid, 3,5-bis(aminomethyl)benzoic acid (Bab), 4-aminomethylphenylalaline (Amp), 4R-4-aminoproline (Apr), 4-(2-aminoethoxy)phenylalanine, 4-aminopiperidine-4-carboxylic acid (Apc), 2-((1,3-diaminopropan-2-yl)oxy)acetic acid (Dpa), or a D enantiomer thereof. In some embodiments, T comprises a first terminal functionality, a second terminal functionality and a third terminal functionality, which are the same or different and bind to L^{Z(i)}, L^{A} (or alternatively A), and L^{E} (or alternatively E), respectively to independently form an amide group, an amine group, a urea group, a thioether group, a thiourea group or a thioamide group. In some embodiments, T binds to L^{A} (or alternatively A), and/or L^{E} (or alternatively E) to further form an ester group, a thioester group, a carbonate group, a carbamate group, a disulfide bond, a hydrazone group, or an oxime group.

In some embodiments, the compound is selected from any one of Table 2.

In one aspect, provided herein is a conjugate, comprising the structure of formula (I):

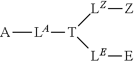

wherein:
(1) T is a branching group which is at least trivalent,
(2) Z is a target binding moiety specifically binding to a target expressed on a cell, wherein Z has a molecular size of less than 66 kDa and is conjugated to T via a linker L^{Z},
(3) E is an effector moiety, wherein E is conjugated to T via a linker L^{E}, and
(4) A is a potency enhancing moiety, wherein A is conjugated to T via a linker L^{A},
wherein A, L^{A}, E, L^{E}, and T are as defined herein.

In some embodiments, the potency enhanced by A includes improved binding affinity against the tumor antigen that Z specifically binds to, extended in vivo half-life, increased tumor uptake, and/or enhanced in vivo activity.

In some embodiments, E comprises a chelating group complexed with a radionuclide. In some embodiments, the radionuclide is a radioactive isotope selected from the group consisting of As, Se, K, Sc, Ti, Cr, Mn, Fe, Co, Ni, Cu, Ga, Ge, Rb, Sr, Y, Zr, Nb, Tc, Rh, Pd, In, Sn, Sb, Zn, Ta, W, Re, Os, Ir, Pt, Au, Hg, Tl, Pb, Bi, Po, Fr, Pm, a lanthanide, an actinide, Mg, Al, Ca, Cd, and Ba. In some embodiments, the radioactive isotope is lanthanide, and wherein the lanthanide is Lu, Sm, Ho, or Tb. In some embodiments, the radioactive isotope is actinide, and wherein the actinide is Ac, Th, or U. In some embodiments, the one or more radionuclides are each independently selected from the group consisting of ⁹⁹Tc, ^{99m}Tc, ¹⁸⁸Re, ¹⁸⁶Re, ¹⁵³Sm, ⁶⁶Ga ⁶⁷Ga, ⁶⁸Ga, ¹¹In, ¹²³In ⁵⁹Fe, ⁶³Zn, ⁵²Fe, ⁵²Mn, ⁴⁵Ti, ⁶⁰Cu, ⁶¹Cu, ⁶⁷Cu, ⁶⁴Cu, ⁶²Cu, ⁸²Rb, ^{195m}Pt, ^{191m}Pt, ^{193m}Pt, ^{117m}Sn, ⁸⁹Zr, ¹⁷⁷Lu, ¹⁸F, ¹⁸⁸Re, ¹⁸⁶Re, ¹⁵³Sm, ⁶⁶Ho, ⁸⁶Y, ⁸⁷Y, ⁹⁰Y, ⁸⁹Sr, ¹⁵³Gd, ¹⁵⁹Gd, ²²⁵Ac, ²¹²Bi, ²¹³Bi, ¹⁹⁸Au, ¹⁹⁹Au, ^{193m}Pt, ¹⁹⁷Pt, ¹⁰³Pd, ¹⁰⁹Pd, ¹⁰⁵Rh, ^{101m}Rh, ^{103m}Rh, ²²³Ra, ²²⁴Ra, ⁹⁷Ru, ²²⁷Th, ²²⁹Th, ¹⁶¹Tb, ¹⁴⁹Tb, ²⁰³Pb, ²¹²Pb, ²⁰¹TI, ¹¹⁹Sb, ^{5m8}Co, Co, ⁵⁷Co, ⁴⁷Sc, ¹⁴⁹Pm, ¹⁴²Pr, ¹⁶¹Ho, ¹⁶⁶Ho, ¹⁷⁵Yb, and ⁵¹Cr. In some embodiments, E is ¹⁷⁷Lu-DOTA-, ¹⁷⁷Lu-DOTAGA-, ²²⁵Ac-DOTA-, or ²²⁵Ac-DOTAGA-. In some embodiments, the target Z specifically binds to is a tumor antigen expressed on a tumor cell. In some embodiments, the target is selected from the group consisting of Carbonic Anhydrase IX (CAIX), B7 Homolog 3 (B7-H3), Guanylyl cyclase C (GCC), prostate specific membrane antigen (PSMA), fibroblast activation protein alpha (FAP-alpha, FAP), Epidermal Growth Factor Receptor (EGFR), Human Epidermal Growth Factor Receptor-2 (HER-2), Receptor tyrosine-protein kinase erbB-3 (HER-3), Platelet-derived growth factor receptor beta (PDGF-Rβ), Programmed death-ligand 1 (PD-L1), Tumour Necrosis Factor alpha (TNF-α), Vascular endothelial growth factor receptor 2 (VEGFR-2), Vascular Endothelial Growth Factor (VGFR), Vascular Endothelial Growth Factor Receptor (VEGFR), Claudin 18.2, Glucagon-like Peptide-1 Receptor (GLP-1R), Glucose-dependent Insulinotropic Polypeptide Receptor (GIP-R), Cholecystokinin-2 Receptor (CCK2R), Gastrin Releasing Peptide Receptor (GRPR), Somatostatin Receptor 2 (SSTR2), Neurotensin Receptor 1 (NTR1), Neuropeptide Y Receptor Type 1 (Y1R), Nectin-4, Epithelial Cell Adhesion Molecule (EpCAM), and Insulin-like Growth Factor-1 (IGF-1) Receptor. In some embodiments, Z comprises an antibody or antibody mimetic selected from the group consisting of a single domain antibody (sdAb), a fragment antigen-binding region (Fab), a single-chain variable fragment (scFv), an affibody, a DARPin (an acronym for designed ankyrin repeat proteins), an Anticalin, an Avimer (avidity multimer), and a Versabody. In some embodiments, L^{Z} is covalently conjugated with a Cys or Lys amino acid residue of Z. In some embodiments, L^{Z} is covalently conjugated with a Cys amino acid residue of Z by a structure of L^{Z} selected from the group consisting of

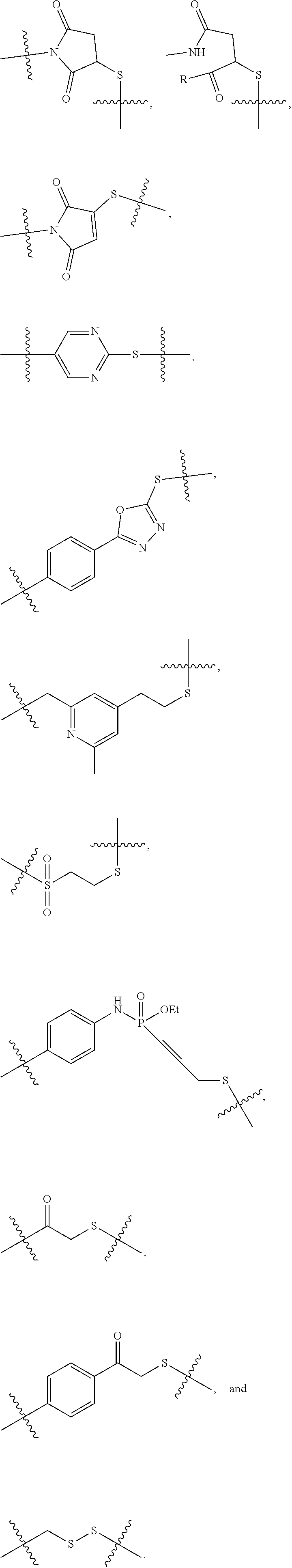

In some embodiments, L^{Z} is covalently conjugated with a Lys amino acid residue of Z by a structure L^{Z} selected from the group consisting of

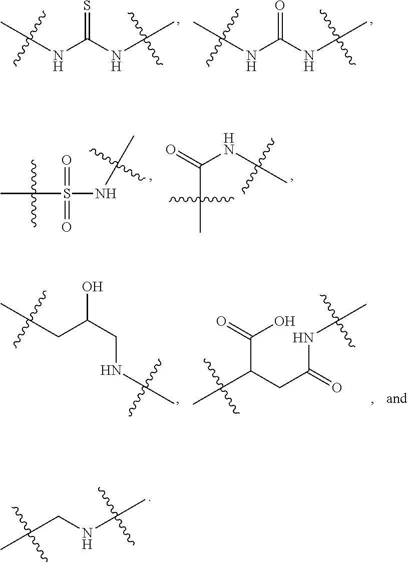

In some embodiments, Z comprises a structure of formula (XI):

B—P (XI),

wherein:

B is an antibody or antibody mimetic selected from a single domain antibody (sdAb), a fragment antigen-binding region (Fab), a single-chain variable fragment (scFv), an affibody, a DARPin (an acronym for designed ankyrin repeat proteins), an Anticalin, an Avimer (avidity multimer), or a Versabody; and P is null or comprises a structure of formula (XII):

X—J—Y (XII),

wherein X is null or an amino acid linker, J is a Cys residue, and Y is null or an extension moiety, and wherein X or Y do not contain an amino acid residue that is identical to J.

In some embodiments, B is a single domain antibody specifically binding to a tumor antigen selected from the group consisting of Guanylyl cyclase C (GCC), prostate specific membrane antigen (PSMA), fibroblast activation protein alpha (FAP-alpha, FAP), Epidermal Growth Factor Receptor (EGFR), Human Epidermal Growth Factor Receptor-2 (HER-2), Vascular Endothelial Growth Factor (VGFR), Vascular Endothelial Growth Factor Receptor (VEGFR), Claudin 18.2, Glucagon-like Peptide-1 Receptor (GLP-1R), Glucose-dependent Insulinotropic Polypeptide Receptor (GIP-R), Cholecystokinin-2 Receptor (CCK2R), Gastrin Releasing Peptide Receptor (GRPR), Somatostatin Receptor 2 (SSTR2), Neurotensin Receptor 1 (NTR1), Neuropeptide Y Receptor Type 1 (Y1R), Nectin-4, Epithelial Cell Adhesion Molecule (EpCAM), and Insulin-like Growth Factor-1 (IGF-1) Receptor.

In some embodiments, B is a single domain antibody comprising:
(1) a CDR1 comprising an amino acid sequence as set forth in SEQ ID NO. 6, 11, or 19, or an amino acid sequence with one to three amino acid alterations as compared to SEQ ID NO. 6, 11, or 19,
(2) a CDR2 comprising an amino acid sequence as set forth in SEQ ID NO. 7, 12, or 20, or an amino acid sequence with one to three amino acid alterations as compared to SEQ ID NO. 7, 12, or 20, and
(3) a CDR3 comprising an amino acid sequence as set forth in SEQ ID NO. 8, 13, or 21, or an amino acid sequence with one to three amino acid alterations as compared to SEQ ID NO. 8, 13, or 21.

In some embodiments, the sdAb comprises an amino acid sequence having at least 80% identity with SEQ ID NO. 5, 10, or 18.

In some embodiments, B is an affibody specifically binding to a tumor antigen selected from Carbonic Anhydrase IX (CAIX), Epidermal growth factor receptor (EGFR), Receptor tyrosine-protein kinase erbB-2 (HER-2), Receptor tyrosine-protein kinase erbB-3 (HER-3), Insulin-like growth factor 1 (IGF-1R), Platelet-derived growth factor receptor beta (PDGF-Rβ), Programmed death-ligand 1 (PD-L1), Tumour Necrosis Factor alpha (TNF-α), Vascular endothelial growth factor receptor 2 (VEGFR-2) or B7 Homolog 3 (B7-H3).

In some embodiments, B is an affibody comprising an amino acid sequence as set forth in SEQ ID NO. 23:

(SEQ ID NO. 23)
AEAKXAKEKIX₁₁ALX₁₄EIIWLPNLTX₁₉X₂₀QIX₂₃AFIAX₂₈LNDDP
SQSSELLSEAKKLX₄₇X₄₈SQ,

wherein
X₅ is selected from Phe or Tyr;
X₁₁ is selected from Lys, Asn or Ala;
X₁₄ is selected from Ser or Gly;
X₁₉ is selected from Tyr or His;
X₂₀ is selected from Gly or Asp;
X₂₃ is selected from Lys or Met;
X₂₈ is selected from Lys or Ala;
X₄₇ is selected from Ser or Asn;
X₄₈ is selected from Glu or Asp;
X₅₁ is selected from null or Gly;
X₅₂ is selected from null or Gly;
X₅₃ is selected from null or Gly;
X₅₄ is selected from Cys or Lys; and
X₅₅ is selected from null or Ala.

In some embodiments, B is an affibody comprising an amino acid sequence having at least 80% identity with SEQ ID NO. 24.

In some embodiments, P is at C-terminus of B. In some embodiments, P comprises no more than 20 amino acid residues.

In some embodiments, X comprises an amino acid sequence selected from (GGS)ₙ, (GSG)ₙ, (GGGS)ₙ or (GGGGS)ₙ, (GGG)ₙ, (EAAAK)ₙ, and n is any integer from 0-3. In some embodiments, X comprises an amino acid sequence selected from GGG, GGS, GSG, GGGS or GGGGS.

In some embodiments, Y comprises no more than 5 amino acids. In some embodiments, Y comprises an amino acid residue adjacent to J with polar uncharged side chain or positive charged side chain. In some embodiments, the amino acid residue adjacent to J is selected from the group consisting of Ala, Ser, Thr, Gln, Asn, Pro, His, and Arg.

In some embodiments, P comprises an amino acid sequence of anyone selected from the group consisting of GGGC, GGSC, GGGSC, GGGCA, GGGSCA, GGSCA, GGGSCSA, GGGSCYA, GGGSCQA, GGGSCPA, GGGSCHA, GGSCSA, GGSCYA, GGSCQA, GGSCPA, GGSCHA, GGGCSA, GGGCYA, GGGCQA, GGGCPA, GGGCHA, GGGGSCSA, GGGGSCYA, GGGGSCQA, GGGGSCPA, GGGGSCHA, GSGCSA, GSGCYA, GSGCQA, GSGCPA, and GSGCHA.

In some embodiments, L^{Z} is covalently conjugated with the thiol group of J by a structure of L^{Z} selected from the group consisting of

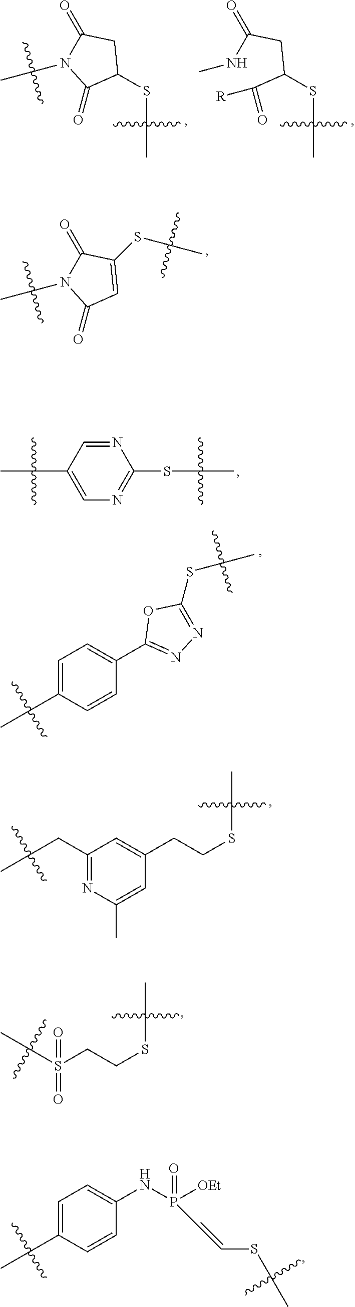

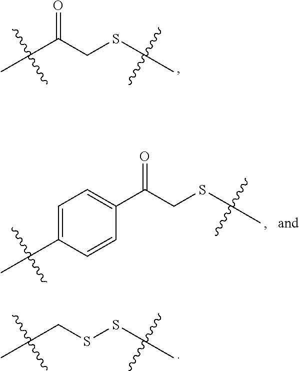

In some embodiments, the conjugate comprises a structure selected from any one of Table 3.

In one aspect, provided herein is a method of preparing the conjugate described herein, comprising:
(i) introducing into a host cell a nucleic acid encoding Z,
(ii) culturing the host cell under conditions that allow expression of Z;
(iii) isolating Z; and
(iv) conjugating Z with the compound of formula (I-X) via L^{Z(i)} of the compound:

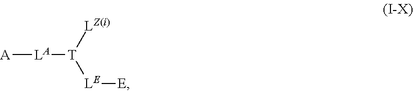

wherein:
(1) L^{Z(i)} is a linker comprising a thiol-reactive group or an amine-reactive group,
(2) T is a branching group which is at least trivalent,
(3) E is an effector moiety, wherein E is conjugated to T via a linker L^{E}, and
(4) A is a potency enhancing moiety, wherein A is conjugated to T via a linker L^{A}, wherein A, L^{A}, E, L^{E}, L^{Z}(i), T and Z are as defined herein.

In some embodiments, the host cell is a eukaryotic cell or a prokaryotic cell. In some embodiments, the eukaryotic cell is a mammalian cell.

In one aspect, provided herein is a pharmaceutical composition, comprising the conjugate described herein and a pharmaceutically acceptable excipient.

In one aspect, provided herein is a kit, comprising the conjugate described herein, and instructions for using the kit to diagnose a disease or disorder in a subject in need thereof.

In one aspect, provided herein is a method of treating a disease or disorder in a subject in need thereof, comprising administering to the subject a pharmaceutical effective amount of the conjugate described herein.

In one aspect, provided herein is a method of inhibiting proliferative activity in a cell, comprising administering an effective amount of the conjugate described herein.

In one aspect, provided herein is a method of imaging a tissue in a subject by administering an imaging effective amount a pharmaceutical effective amount of the conjugate described herein, to a subject in need thereof and applying an imaging technique to detect emitted gamma rays.

In one aspect, provided herein is a method of diagnosing cancer in subject by administering a diagnostic effective amount a pharmaceutical effective amount of the conjugate described herein and applying an imaging technique to detect emitted gamma rays.

### BRIEF DESCRIPTION OF THE DRAWINGS

Certain embodiments of the application will now be described in greater detail with reference to the attached drawings in which:

FIGS. 1A-1B illustrate mass spec. results of exemplary conjugates of the present application.

FIGS. 2A-2B illustrate biodistribution profile of exemplary sdAb conjugates targeting Claudin-18.2.

FIGS. 3A-3D illustrate biodistribution profile of exemplary sdAb conjugates targeting FAP.

FIGS. 4A-4D illustrate biodistribution profile of exemplary affibody conjugates targeting B7-H3.

### DETAILED DESCRIPTION

### Definition

Unless otherwise indicated, the definitions and embodiments described in this and other sections are intended to be applicable to all embodiments and aspects of the present application herein described for which they are suitable as would be understood by a person skilled in the art.

All features disclosed in the specification, including the claims, abstract, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent, or similar purpose, unless expressly stated otherwise.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

As used in the present application, the singular forms “a”, “an” and “the” include plural references unless the content clearly dictates otherwise. For example, an embodiment including “a group” should be understood to present certain aspects with one group, or two or more additional groups. an embodiment including “a group” should be understood to present certain aspects with one group, or two or more additional groups.

In embodiments comprising an “additional” or “second” component or effect, such as an additional or second conjugate, the second conjugate as used herein is different from the other conjugates or first conjugate. A “third” conjugate is different from the other, first, and second conjugates, and further enumerated or “additional” conjugates are similarly different.

The term “and/or” as used herein means that the listed items are present, or used, individually or in combination. In effect, this term means that “at least one of” or “one or more” of the listed items is used or present.

As described herein, “tumor antigen” refers to an antigenic substance produced at a tumor site.

As described herein, the term “antibody” is used in its broadest sense and encompasses various antibody structures, including but not limited to monoclonal antibodies, polyclonal antibodies, full-length antibodies and antigen-binding fragments thereof, so long as they exhibit the desired antigen-binding activity.

A full-length antibody comprises two heavy chains and two light chains. The variable regions of the light and heavy chains are responsible for antigen binding. The variable domains of the heavy chain and light chain may be referred to as “VH” and “VL”, respectively. The variable regions in both chains generally contain three highly variable loops called the complementarity determining regions (CDRs) (light chain (LC) CDRs including LC-CDR1, LC-CDR2, and LC-CDR3, heavy chain (HC) CDRs including HC-CDR1, HC-CDR2, and HC-CDR3). CDR boundaries for the antibodies and antigen-binding fragments disclosed herein may be defined or identified by the conventions of IMGT, Kabat, or Chothia. The three CDRs of the heavy or light chains are interposed between flanking stretches known as framework regions (FRs), which are more highly conserved than the CDRs and form a scaffold to support the hypervariable loops. The constant regions of the heavy and light chains are not involved in antigen binding but exhibit various effector functions. Antibodies are assigned to classes based on the amino acid sequence of the constant region of their heavy chain. The five major classes or isotypes of antibodies are IgA, IgD, IgE, IgG, and IgM, which are characterized by the presence of α, δ, δ, γ, and μ heavy chains, respectively. Several of the major antibody classes are divided into subclasses such as IgG1 (γ1 heavy chain), IgG2 (γ2 heavy chain), IgG3 (γ3 heavy chain), IgG4 (γ4 heavy chain), IgA1 (al heavy chain), or IgA2 (α2 heavy chain).

The term “antigen-binding fragment” as used herein refers to an antibody fragment including, for example, an Fab, an Fab′, an Fv fragment, a disulfide stabilized Fv fragment (dsFv), a single-chain Fv (scFv), a single domain antibody (e.g., a camelized single domain antibody), a nanobody, a domain antibody, or any other antibody fragment that binds to an antigen but does not comprise a complete antibody structure. An antigen-binding fragment is capable of binding to the same antigen to which the parent antibody or a parent antibody fragment (e.g., a parent scFv) binds. In some embodiments, an antigen-binding fragment may comprise one or more CDRs from a particular human antibody grafted to a framework region from one or more different human antibodies.

The term “single-domain antibody (sdAb, also known as nanobody)” as used herein refers to an antibody fragment consisting of a single monomeric variable antibody domain. Like a whole antibody, it is capable of selectively binding to a specific antigen. With a molecular weight of only 12-15 kDa, single-domain antibodies are much smaller than full-length antibodies (150-160 kDa), and even smaller than Fab fragments (~50 kDa, one light chain and half a heavy chain) and single-chain variable fragments (~25 kDa, two variable domains, one from a light and one from a heavy chain).

The first single-domain antibodies were engineered from heavy-chain antibodies found in camelids, which are also named as “VHH” fragments. Cartilaginous fishes also have heavy-chain antibodies (IgNAR, immunoglobulin new antigen receptor), from which single-domain antibodies called VNAR fragments can be obtained. An alternative approach is to split the dimeric variable domains from common immunoglobulin G (IgG) from humans or mice into monomers. Although most researches into single-domain antibodies are currently based on heavy chain variable domains, nanobodies derived from light chains are also capable of binding specifically to target epitopes.

Single-domain antibodies have been shown to be as specific as full-length antibodies, and in some cases, they are even more robust. Single-domain antibodies can be easily isolated using the same phage panning procedure used for antibodies. In addition, the smaller size and single domain allow it easier to transform into bacterial cells for bulk production, making single-domain antibody ideal for pharmaceutical purpose.

As used herein, the term “CDR” or “complementarity determining region” is intended to mean the non-contiguous antigen combining sites found within the variable region of both heavy and light chain polypeptides. These particular regions have been described by Kabat, et al. (1977) J. Biol. Chem. 252:6609-6616; Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Plickthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. The amino acid residues which encompass the CDRs as defined by each of the above cited references are set forth below in Table 1 as a comparison. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present application and for possible inclusion in one or more claims herein.

TABLE 1
CDR Definitions

| | Kabat¹ | Chothia² | MacCallum³ | IMGT⁴ | AHo⁵ |
|---|---|---|---|---|---|
| VH CDR1 | 31-35 | 26-32 | 30-35 | 27-38 | 25-40 |
| VH CDR2 | 50-65 | 53-55 | 47-58 | 56-65 | 58-77 |
| VH CDR3 | 95-102 | 96-101 | 93-101 | 105-117 | 109-137 |
| VL CDR1 | 24-34 | 26-32 | 30-36 | 27-38 | 25-40 |
| VL CDR2 | 50-56 | 50-52 | 46-55 | 56-65 | 58-77 |
| VL CDR3 | 89-97 | 91-96 | 89-96 | 105-117 | 109-137 |

¹Residue numbering follows the nomenclature of Kabat et al., supra
²Residue numbering follows the nomenclature of Chothia et al., supra
³Residue numbering follows the nomenclature of MacCallum et al., supra
⁴Residue numbering follows the nomenclature of Lefranc et al., supra
⁵Residue numbering follows the nomenclature of Honegger and Plückthun, supra

In some embodiments, the CDRs of an antibody can be determined according to the IMGT numbering system. With respect to the IMGT numbering system, (i) the VH CDR1 is typically present at amino acid positions 25 to 35 of the heavy chain; (ii) the VH CDR2 is typically present at amino acid positions 51 to 57 of the heavy chain; and (iii) the VH CDR2 is typically present at amino acid positions 93 to 102 of the heavy chain. With respect to the IMGT numbering system, (i) the VL CDR1 is typically present at amino acid positions 27 to 32 of the light chain; (ii) the VL CDR2 is typically present at amino acid positions 50 to 52 of the light chain; and (iii) the VL CDR3 is typically present at amino acid positions 89 to 97 of the light chain.

The expression “variable-domain residue-numbering as in Kabat” or “amino-acid position numbering as in Kabat,” and variations thereof, refers to the numbering system used for heavy-chain variable domains or light-chain variable domains of the compilation of antibodies in Kabat et al., supra. Using this numbering system, the actual linear amino acid sequence may contain fewer or additional amino acids corresponding to a shortening of, or insertion into, a FR or hypervariable region (HVR) of the variable domain. For example, a heavy-chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

“Framework” or “FR” residues are those variable-domain residues other than the CDR residues as herein defined.

“Percent (%) amino acid sequence identity” or “homology” with respect to the polypeptide and antibody sequences identified herein is defined as the percentage of amino acid residues in a candidate sequence that are identical with the amino acid residues in the polypeptide or antibody being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32(5):1792-1797, 2004; Edgar, R. C., BMC Bioinformatics 5(1):113, 2004).

The term “Fc region” or “fragment crystallizable region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies described herein include human IgG1, IgG2 (IgG2A, IgG2B), IgG3 and IgG4.

As used herein, the term “antibody mimetic” refers to a molecule capable of mimicking an antibody's ability to bind an antigen, but not limited to native antibody structure. Examples of such antibody mimetics include, but are not limited to, affibodies, DARPins, Anticalins, Avimers, and Versabodies.

Affibody molecules represent a new class of affmity proteins based on a 58-amino acid residue protein domain, derived from one of the IgG-binding domains of staphylococcal protein A. This three-helix bundle domain has been used as a scaffold for the construction of combinatorial phagemid libraries, from which affibody variants that target the desired molecules can be selected using phage display technology (Nord K, Gunneriusson E, Ringdahl J, Stahl S, Uhlen M, Nygren PA, Binding proteins selected from the group consisting of combinatorial libraries of an α-helical bacterial receptor domain, Nat Biotechnol 1997; 15:772-7. Ronmark J, Gronlund H, Uhlen M, Nygren PA, Human immunoglobulin A (IgA) -specific ligands from combinatorial engineering of protein A, Eur J Biochem 2002; 269:2647-55.). The simple, robust structure of affibody molecules in combination with their low molecular weight (~6 kDa), make them suitable for a wide variety of applications, for instance, as detection reagents (Ronmark J, Hansson M, Nguyen T, et al, Construction and characterization of affibody-Fc chimeras produced in Escherichia coli, J Immunol Methods 2002; 261:199-211) and to inhibit receptor interactions (Sandstorm K, Xu Z, Forsberg G, Nygren PA, Inhibition of the CD28-CD80 co-stimulation signal by a CD28-binding Affibody ligand developed by combinatorial protein engineering, Protein Eng 2003; 16:691-7). Further details of affibodies and methods of production thereof may be obtained by reference to U.S. Pat. No. 5,831,012 which is herein incorporated by reference in its entirety.

DARPin (Designed Ankyrin Repeat Proteins) is an example of antibody mimetic DRP (Designed Repeat Protein) technology that has been developed to exploit the binding abilities of non-antibody polypeptides. Repeat proteins such as ankyrin or leucine-rich repeat proteins, are ubiquitous binding molecules, which occur, unlike antibodies, intra- and extracellularly. Their unique modular architecture features repeating structural units (repeats), which stack together to form elongated repeat domains displaying variable and modular target-binding surfaces. Based on this modularity, combinatorial libraries of polypeptides with highly diversified binding specificities can be generated. This strategy includes the consensus design of self-compatible repeats displaying variable surface residues and their random assembly into repeat domains. Additional information regarding DARPins and other DRP technologies can be found in US Patent Application Publication No. 2004/0132028, and International Patent Application Publication No. WO 02/20565, both of which are hereby incorporated by reference in their entirety.

Anticalins are derived from lipocalins, a family of low molecular weight proteins that are naturally and abundantly expressed in human tissues and body fluids. Anticalins can also be formatted as dual targeting proteins, so-called Duocalins. A Duocalin binds two separate therapeutic targets in one easily produced monomeric protein using standard manufacturing processes while retaining target specificity and affmity regardless of the structural orientation of its two binding domains. Additional information regarding Anticalins can be found in U.S. Pat. No. 7,250,297 and International Patent Application Publication No. WO 99/16873, both of which are hereby incorporated by reference in their entirety.

Another antibody mimetic technology useful in the present application is Avimer. Avimers are evolved from a large family of human extracellular receptor domains by in vitro exon shuffling and phage display, generating multidomain proteins with binding and inhibitory properties. Linking multiple independent binding domains has been shown to create avidity and results in improved affmity and specificity compared with conventional single-epitope binding proteins. Other potential advantages include simple and efficient production of multitarget-specific molecules in E. coli, improved thermostability and resistance to proteases. Avimers with sub-nanomolar affmities have been obtained against a variety of targets. Additional information regarding Avimers can be found in US Patent Application Publication Nos. 2006/0286603, 2006/0234299, 2006/0223114, 2006/0177831, 2006/0008844, 2005/0221384, 2005/0164301, 2005/0089932, 2005/0053973, 2005/0048512, 2004/0175756, all of which are hereby incorporated by reference in their entirety.

Versabody is another antibody mimetic technology that could be used in the present application. Versabodies are small proteins of 3-5 kDa with >15% cysteines, which form a high disulfide density scaffold, replacing the hydrophobic core that typical proteins have. Given the structure of Versabodies, these antibody mimetics offer a versatile format that includes multi-valency, multi-specificity, a diversity of half-life mechanisms, tissue targeting modules and the absence of the antibody Fc region. Furthermore, Versabodies are manufactured in E. coli at high yields, and because of their hydrophilicity and small size, Versabodies are highly soluble and can be formulated to high concentrations. Versabodies are exceptionally heat stable (they can be boiled) and offer extended shelf-life. Additional information regarding Versabodies can be found in US Patent Application Publication No. 2007/0191272 which is hereby incorporated by reference in its entirety.

The term “epitope” as used herein refers to the specific group of atoms or amino acids on an antigen to which an antibody or antibody moiety binds. Two antibodies or antibody moieties may bind the same epitope within an antigen if they exhibit competitive binding for the antigen.

As used herein, the terms “specifically binds,” “specifically recognizing,” and “is specific for” refer to measurable and reproducible interactions, such as binding between an antibody and an antigen thereof, which is determinative of the presence of the target or antigen in the presence of a heterogeneous population of molecules, including biological molecules. For example, an antibody that specifically recognizes an antigen is the antibody that binds this antigen with greater affinity, avidity, more readily, and/or with greater duration than its bindings to other targets or antigens. In some embodiments, the extent of binding of antibody to an unrelated target or antigen is less than about 10% of the binding of the antibody to the antigen thereof as measured, e.g., by a radioimmunoassay (RIA). In some embodiments, an antibody that specifically binds the antigen thereof has a dissociation constant (K_{D}) of ≤10⁻⁸ M, ≤10⁻⁹ M, ≤10⁻¹¹¹ M, ≤10⁻¹² M, or <10⁻¹² M. In some embodiments, said specific binding can include, but does not require exclusive binding. Binding specificity of the antibody can be determined experimentally by methods known in the art. Such methods comprise, but are not limited to, e.g., Western blots, ELISA-, RIA-, ECL-, IRMA-, EIA-, BIACORE™-tests and peptide scans.

As used herein, the term “conjugate” refers to a molecule which comprises more than one functional moieties, in which a first functional moiety and a second functional moiety are linked (e.g. covalently linked) to one another via a linker, a spacer or a reactive linking group, as described herein. In some embodiments, the term “conjugate(s) of the application” or “conjugate(s) of the present application” and the like as used herein include but are not limited to the conjugates of Formula I, I-A, I-B, I-C, I-D, I-E, I-F, I-G, I-H, I-I, I-J, I-K, I-L, I-A′, I-A′(a), I-A′(b), I-A′(c), I-A′(d), I-B′, I-C′ and I-D′.

The term “composition of the application” or “composition of the present application” and the like as used herein refers to a composition comprising one or more conjugates of the application.

The term “radionuclide” as used herein refers to any atom capable of undergoing radioactive decay. The term radionuclide is used synonymously herein with radioactive nuclide, radioisotope, and radioactive isotope.

The term “suitable” as used herein means that the selection of the particular conjugate or conditions would depend on the specific synthetic manipulation to be performed, the identity of the molecule(s) to be transformed and/or the specific use for the conjugate, but the selection would be well within the skill of a person trained in the art.

The present description refers to a number of chemical terms and abbreviations used by those skilled in the art. Nevertheless, definitions of selected terms are provided for clarity and consistency.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas).

As used herein, the term “PEG” or “polyethylene glycol” refers to a water-soluble poly(ethylene oxide) with the structure “—(OCH₂CH₂)ₙ—.” As used herein, PEG also includes “—CH₂CH₂—O(CH₂CH₂O)ₙ—CH₂CH₂—” and “—(OCH₂CH₂)ₙO—,” depending upon whether or not the terminal oxygens have been displaced. In some embodiments, n is in a range from about 1 to about 28. Throughout the present application, it should be understood that the term “PEG” includes structures having various terminal groups and so forth. The term “PEG” also means a polymer that contains a majority, that is to say, greater than 50%, of —OCH₂CH₂— repeating subunits. With respect to specific forms, the PEG encompasses those branched, linear, and forked structures.

The term “alkyl” as used herein, whether it is used alone or as part of another group, means straight or branched chain, saturated alkyl groups. The numbers of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms.

All alkyl groups are optionally substituted unless otherwise indicated.

The term “alkylene”, whether it is used alone or as part of another group, means straight or branched chain, saturated alkylene group, that is, a saturated carbon chain that contains substituents on two of its ends. The numbers of carbon atoms that are possible in the referenced alkylene group are indicated by the prefix “Cₙ₁₋ₙ₂”. For example, the term C₂₋₆ alkylene means an alkylene group having 2, 3, 4, 5 or 6 carbon atoms. All alkylene groups are optionally substituted unless otherwise indicated.

The term “alkenyl” as used herein, whether it is used alone or as part of another group, means straight or branched chain, unsaturated alkyl groups containing at least one double bond. The number of carbon atoms that are possible in the referenced alkenyl group are indicated by the prefix “Cₙ₁₋ₙ₂”. For example, the term C₂₋₆alkenyl means an alkenyl group having 2, 3, 4, 5 or 6 carbon atoms and at least one double bond. All alkenyl groups are optionally substituted unless otherwise indicated.

The term “alkenylene”, whether it is used alone or as part of another group, means straight or branched chain, unsaturated alkyl groups containing at least one double bond that contains substituents on two of its ends. The numbers of carbon atoms that are possible in the referenced alkenylene group are indicated by the prefix “Cₙ₁₋ₙ₂”. For example, the term C₂₋₆alkenylene means an alkenylene group having 2, 3, 4, 5 or 6 carbon atoms. All alkenylene groups are optionally substituted unless otherwise indicated.

The term “aryl” as used herein, whether it is used alone or as part of another group, refers to carbocyclic groups containing at least one aromatic ring and contains 6 to 20 carbon atoms.

The term “cycloalkyl,” as used herein, whether it is used alone or as part of another group, means a saturated carbocyclic group containing from 3 to 20 carbon atoms and one or more rings. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms.

The term “heterocycloalkyl” as used herein, whether it is used alone or as part of another group, refers to cyclic groups containing at least one non-aromatic ring containing from 3 to 20 atoms in which one or more of the atoms are a heteroatom selected from the group consisting of O, S and N and the remaining atoms are C. Heterocycloalkyl groups are either saturated or unsaturated (i.e. contain one or more double bonds). When a heterocycloalkyl group contains the prefix Cₙ₁₋ₙ₂ this prefix indicates the number of carbon atoms in the corresponding carbocyclic group, in which one or more, suitably 1 to 5, of the ring atoms is replaced with a heteroatom as selected from the group consisting of O, S and N and the remaining atoms are C. Heterocycloalkyl groups are optionally benzofused.

The term “heteroaryl” as used herein, whether it is used alone or as part of another group, refers to cyclic groups containing at least one heteroaromatic ring containing 5-20 atoms in which one or more of the atoms are a heteroatom selected from the group consisting of O, S and N and the remaining atoms are C. When a heteroaryl group contains the prefix Cₙ₁₋ₙ₂ this prefix indicates the number of carbon atoms in the corresponding carbocyclic group, in which one or more, suitably 1 to 5, of the ring atoms is replaced with a heteroatom as defined above. Heteroaryl groups are optionally benzofused.

All cyclic groups, including aryl, heteroaryl, heterocycloalkyl and cycloalkyl groups, contain one or more than one ring (i.e. are polycyclic). When a cyclic group contains more than one ring, the rings may be fused, bridged, spirofused or linked by a bond.

The term “target binding moiety” as used herein refers to a moiety that is recognized by a target site to which it binds.

The term “target” or “target site” as used herein means a receptor, for example a cell surface receptor, antigen, for example Claudin 18.2, GCC, FAP, B7-H3 or other protein on a cell surface to which a target binding group can bind.

The term “chelating group” as used herein is chelator moiety capable of complexing a radionuclide.

The term “branching group that is at least trivalent” as used herein refers to any molecular structure that comprises at least three terminal functional groups and each terminal functional group connects with another molecular structure. The at least three terminal functional groups can be the same or different.

The term “half-life” as used herein refers to a pharmacokinetic property of a conjugate and is a measure of the mean survival time of the conjugate following administration of the conjugate to a subject. Half-life is the time required for half the quantity of the conjugate administered in a subject to be cleared from the subject's body or a specific compartment thereof (e.g serum or in other tissue).

The term “in vivo half-life” as used herein refers to the time required for half the quantity of a conjugate administered to a subject to be cleared from the circulation (e.g blood) and/or other tissue of the subject.

The term “ex vivo plasma half-life” as used herein refers to the time required for half the quantity of a conjugate that has been combined with plasma, for example, mouse plasma, to be degraded within the plasma at about 37° C. and at neutral pH. Ex vivo plasma half-life may be measured for example by incubating a radioligand in mouse plasma at 37° C.

At each desired time point, an aliquot of sample may be taken out, worked up and analyzed by HPLC with radio detection. This measures the remaining % of intact radioligand, which can be plotted to provide a measure of ex vivo plasma half-life.

The term “linker group” as used herein refers to any molecular structure that connects two or more other molecular structures together.

The term “non-cleavable linker group” as used herein refers to any molecular structure that joins two or more other molecular structures together and comprises non-cleavable moieties. The non-cleavable linker group contains functional groups on each of the termini that react with complementary functional groups of the molecules to be linked to form non-cleavable moieties. When a conjugate of the application comprises more than one non-cleavable linker, each non-cleavable linker is an independent non-cleavable linker, and the one or more non-cleavable linkers may be the same or different.

The term “cleavable linker” as used herein refers to any molecular structure that joins two or more other molecular structures together and comprises at least one cleavable moiety. The cleavable linker group contains functional groups on each of the termini that reacts with complementary functional groups of the molecules to be linked to form non-cleavable or cleavable moieties. When a conjugate of the application comprises more than one cleavable linker, each cleavable linker is an independent cleavable linker and the one or more cleavable linkers may be the same or different.

The term “non-cleavable moiety(ies)” as used herein refers to chemical functional groups that resist degradation by one or more of acids, bases, reducing agents oxidizing agents and enzymes. Non-cleavable moieties are generally stable towards cleavage but can be cleavable, for example, by enzymes or physiological conditions inside a cell, tissue, or organ after a period of time and after the conjugate or the portion of the conjugate that contains the non-cleavable moiety is delivered or transported to the target site.

The term “cleavable moiety(ies)” as used herein refers to a chemical functional group that is degradable by one or more acids, bases, reducing agents, oxidizing agents and/or enzymes.

The term “stable towards cleavage”, or “resists degradation” as used herein refers to a chemical functional group that less than about 5% of which is degraded in mouse plasma at about 37° C. after at least about 48 hours following combining of a conjugate comprising the chemical functional group with the mouse plasma.

The term “degraded” or “cleavable” as used herein in relation to the cleavable moiety means that greater than about 5% of the cleavable moiety is degraded in mouse plasma at about 37° C. after at least about 48 hours following combining of a conjugate comprising the cleavable moiety with the mouse plasma.

The term “amino acid residue” as used herein refers to an amino acid without the “—OH” of its carboxyl group and/or the “H” portion of an amino group.

The term “amino acid” as used herein is any structure comprising a carboxyl (—COOH) functional group and an amine (—NH₂) functional group. In some embodiments, the amino acid used herein, unless otherwise specified, encompasses naturally occurring amino acids, unnaturally occurring amino acids, chemically modified naturally or unnaturally occurring amino acids, D enantiomers of the naturally or unnaturally occurring amino acid residues or the modified amino acid residues, amino acid residues derived from a naturally or unnaturally β-amino acid or a γ-amino acid. In some embodiments, “unnatural amino acid”, as used herein, refers to an amino acid that is not a naturally occurring amino acid and is obtained synthetically or by modification of a natural amino acid. In some embodiments, “naturally occurring amino acid” as used herein refers to amino acids that occur naturally and are encoded by the genetic code, as well as those encoded amino acids that are later modified in vivo. It is understood that any of the amino acids or amino acid residues described herein may, in some embodiments, connect to one or more amino acids or amino acid residues described herein via a covalent peptide bond.

The term “at least one” when preceding an item refers to a single member of the item or when preceding a list of items refers to a single member of the item and any combination of those items. For example “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The term “treating” or “treatment” as used herein and as is well understood in the art, means an approach for obtaining beneficial or desired results, including clinical results. Beneficial or desired clinical results include but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. A subject with early cancer can be treated to prevent progression for example, or alternatively a subject in remission can be treated with a conjugate or composition of the application to prevent recurrence. Treatment methods comprise administering to a subject a therapeutically effective amount of one or more of the conjugates of the application and optionally consist of a single administration, or alternatively comprise a series of administrations.

“Palliating” a disease, disorder or condition means that the extent and/or undesirable clinical manifestations of a disease, disorder or condition are lessened and/or time course of the progression is slowed or lengthened, as compared to not treating the disorder.

The terms “preventing”, “prevention” or “prophylaxis”, or synonyms thereto, as used herein refers to a reduction in the risk or probability of a patient becoming afflicted with a disease, disorder or condition or manifesting a symptom associated with a disease, disorder or condition.

As used herein, the term “therapeutically effective amount” means an amount of a conjugate, or one or more conjugates, of the application that is effective, at dosages and for periods of time necessary to achieve the desired result.

The term “imaging effective amount” when used in connection with a one or more conjugates of the application, is an amount of the conjugate that is sufficient to produce a visible image when the conjugate is administered to a subject and the radiation emitted by the conjugate is detected using positron-emission tomography (“PET”) or single photon emission tomography (SPECT) or autoradiography or ex vivo or in vitro binding assays.

As used herein, the term “diagnostic effective amount” means an amount of a conjugate, or one or more conjugates, of the application, that is effective, at dosages and for periods of time necessary to achieve the desired diagnostic effect including, for example, diagnosing a particular condition being assessed.

The term “administered” as used herein means administration of an imaging, diagnostic and/or therapeutically effective amount of one or more conjugates or compositions of the application to a cell, tissue, organ or subject.

The term “cancer” as used herein refers to cellular-proliferative disease states.

The term “subject” as used herein includes all members of the animal kingdom including mammals, and suitably refers to humans. Thus, the methods and uses of the present application are applicable to both human therapy and veterinary applications.

The term “cell” as used herein refers to a single cell or a plurality of cells and includes a cell either in a cell culture or in a subject.

The term “pharmaceutically acceptable” means compatible with the treatment of subjects, for example humans.

The term “pharmaceutically acceptable carrier” means a non-toxic solvent, dispersant, excipient, adjuvant or other material which is mixed with the active ingredient in order to permit the formation of a pharmaceutical composition, i.e., a dosage form capable of administration to a subject.

The term “pharmaceutically acceptable salt” means either an acid addition salt or a base addition salt which is suitable for, or compatible with the treatment of subjects.

An acid addition salt suitable for, or compatible with, the treatment of subjects is any non-toxic organic or inorganic acid addition salt of any basic compound.

A base addition salt suitable for, or compatible with, the treatment of subjects is any non-toxic organic or inorganic base addition salt of any acidic compound.

The term “solvate” as used herein means a compound, or a salt and/or prodrug of a compound, wherein molecules of a suitable solvent are incorporated in the crystal lattice. A suitable solvent is physiologically tolerable at the dosage administered.

The term “ESL1” or “ESL-1” as used herein refers to a cleavable linker group having the chemical formula:

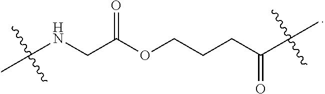 [SMILES: *NCC(=O)OCCCC(*)=O]

The term “ESL2” or “ESL-2” as used herein refers to a cleavable linker group having the chemical formula:

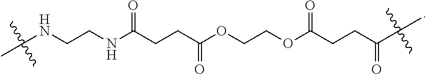 [SMILES: *NCCNC(=O)CCC(=O)OCCOC(=O)CCC(*)=O]

The term “ESL3” or “ESL-3” as used herein refers to a cleavable linker group having the chemical formula:

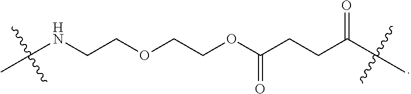 [SMILES: *NCCOCCOC(=O)CCC(*)=O]

The term “SSL1” or “SSL-1” as used herein refers to a cleavable linker group having the chemical formula:

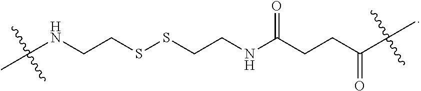 [SMILES: *NCCSSCCNC(=O)CCC(*)=O]

The term “OEG” as used herein refers to a group having the chemical formula:

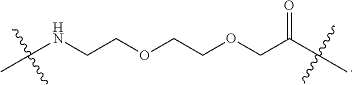 [SMILES: *NCCOCCOCC(*)=O]

The term “2Nal” as used herein refers to a group having the chemical formula:

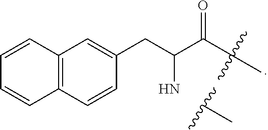

The term “Aoc” as used herein refers to a group having the chemical formula:

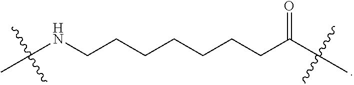 [SMILES: *NCCCCCCCC(*)=O]

The term “Aun” as used herein refers to a group having the chemical formula:

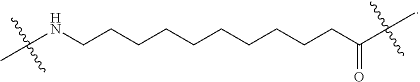 [SMILES: *NCCCCCCCCCCC(*)=O]

The term “Ava” as used herein refers to a group having the chemical formula:

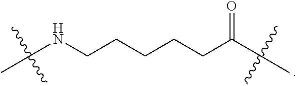 [SMILES: *NCCCCCC(*)=O]

The term “4hBA” as used herein refers to a group having the chemical name 4-hydroxybutanoic acid and having the chemical formula:

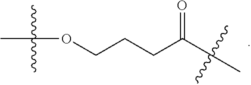 [SMILES: *OCCCC(*)=O]

The term “4hPA-Gly” as used herein refers to a group having the chemical name 4-hydroxybutanoic acid and having the chemical formula:

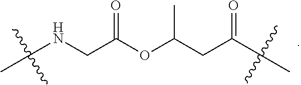 [SMILES: *NCC(=O)OC(C)CC(*)=O]

The term “5hPA-Gly” as used herein refers to a group having the chemical name 4-hydroxybutanoic acid and having the chemical formula:

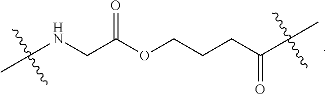 [SMILES: *NCC(=O)OCCCC(*)=O]

The term “4hBA-Leu” as used herein refers to a group having the chemical name 4-hydroxybutanoic acid and having the chemical formula:

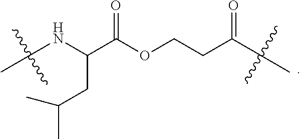 [SMILES: *NC(CC(C)C)C(=O)OCCC(*)=O]

The term “6hHA-Gly” as used herein refers to a group having the chemical name 4-hydroxybutanoic acid and having the chemical formula:

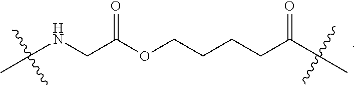 [SMILES: *NCC(=O)OCCCCC(*)=O]

The term “Gly-5aPOH” as used herein refers to a group having the chemical name 4-hydroxybutanoic acid and having the chemical formula:

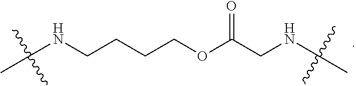 [SMILES: *NCCCCOC(=O)CN*]

The term “I-PADT” as used herein refers to a group having the chemical name 4-hydroxybutanoic acid and having the chemical formula:

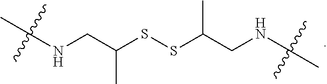 [SMILES: *NCC(C)SSC(C)CN*]

The term “SA-5aPOH” as used herein refers to a group having the chemical name 4-hydroxybutanoic acid and having the chemical formula:

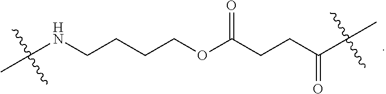 [SMILES: *NCCCCOC(=O)CCC(*)=O]

The term “DAB” as used herein refers to a group having the chemical name 2,4-diaminobutyric acid and having the chemical formula:

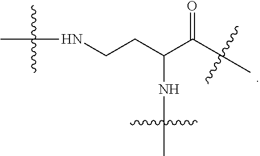 [SMILES: *NCCC(N*)C(*)=O]

The term “DAP” as used herein refers to a group having the chemical name 2,3-diaminopropionic acid and having the chemical formula:

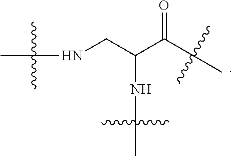 [SMILES: *NCC(N*)C(*)=O]

The term “MAL” as used herein refers to a maleimide group having the chemical formula:

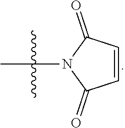 [SMILES: *N1C(=O)C=CC1=O]

The term “Suc” as used herein refers to a thiosuccinimide group having the chemical formula:

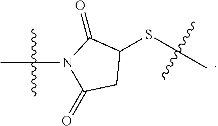 [SMILES: *SC1CC(=O)N(*)C1=O]

The term “p-NCS” as used herein refers to a p-phenylisothiocyanate group having the chemical formula:

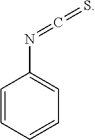

The term “p-PTU” as used herein refers to a p-phenylthiourea group having the chemical formula:

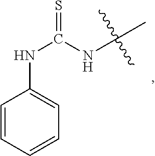 [SMILES: *NC(=S)Nc1ccccc1]

The term “IPhBu” and “4-pIBA” (4-pIPhBu) can be used interchangeably herein and refer to a group having the chemical name 4-iodophenylbutyryl (4-(p-iodophenyl)butyryl) and having the chemical formula:

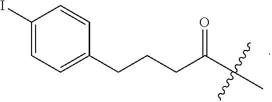 [SMILES: *C(=O)CCCc1ccc(I)cc1]

The term “Ahx” as used herein refers to a linker group having the chemical formula:

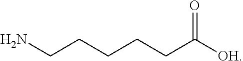

The term “SA-eLys” as used herein refers to a group having the chemical formula:

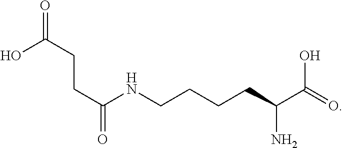 [SMILES: N[C@@H](CCCCNC(=O)CCC(=O)O)C(=O)O]

The term “HO-C14” as used herein refers to a tetradecanedioic acid group having the chemical formula:

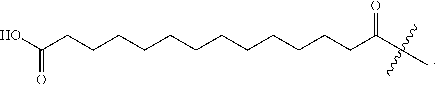 [SMILES: *C(=O)CCCCCCCCCCCCC(=O)O]

The term “HO-C18” as used herein refers to an octadecanedioic acid group having the chemical formula:

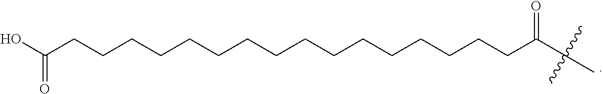 [SMILES: *C(=O)CCCCCCCCCCCCCCCCC(=O)O]

The symbol “ [symbol] ” when drawn perpendicularly across a bond indicates a point of covalent attachment of a chemical group.

When used, for example, with respect to the methods of treatment, uses, compositions, packages and/or kits of the application, a subject, for example a subject “in need thereof” is a subject that would benefit from administration of one or more conjugates of the application, or a pharmaceutically acceptable salt and/or solvate thereof.

### Compound

In one aspect, provided herein is a compound comprising a structure of formula (I-X):

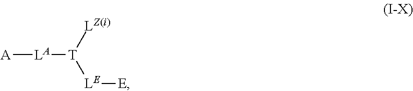

wherein L^{Z(i)} is a linker comprising a thiol-reactive group or an amine-reactive group; T is a branching group which is at least trivalent; E is an effector moiety which is conjugated to T via a linker L^{E}; and A is a potency enhancing moiety which is conjugated to T via a linker L^{A}.

In some embodiments, A of the compound of the present application comprises one or more blood (e.g., plasma) protein binding groups. In some embodiments, the blood (e.g., plasma) protein is selected from the group consisting of albumin, alpha-1-acid glycoprotein, fetuin, transferrin, IgG, HDL (high-density lipoprotein) and LDL (low-density lipoprotein). In some embodiments, A comprises one or more albumin binding groups. In some embodiments, A comprises at least one albumin binding group. In some embodiments, A comprises one albumin binding group. In some embodiments, A comprises at least two albumin binding groups. In some embodiments, A comprises two albumin binding groups. In some embodiments, A comprises two albumin binding groups, and the two albumin binding groups are identical. In some embodiments, A comprises two albumin binding groups, and the two albumin binding groups are different.

Any suitable albumin binding group known in the art can be used in the present application. In some embodiments, the albumin binding group is selected from the group consisting of unsubstituted or substituted C(O)C₁₋₂₆alkyleneCOOH, unsubstituted or substituted C(O)C₁₋₂₆alkenyleneCOOH, unsubstituted or substituted C(O)C₁₋₂₆alkyl, and unsubstituted or substituted C(O)C₁₋₂₆alkenyl. In some embodiments, the albumin binding groups is selected from the group consisting of unsubstituted or substituted C(O)C₆₋₂₀alkyleneCOOH, and unsubstituted or substituted C(O)C₇₋₂₁alkyl. In some embodiments, the albumin binding group is selected from the group consisting of C(O)C₇alkyl, C(O)C₈alkyl, C(O)C₉alkyl, C(O)C₁₀alkyl, C(O)C₁₁alkyl, C(O)C₁₂alkyl, C(O)C₁₃alkyl, C(O)C₁₋₄alkyl, C(O)C₁₅alkyl, C(O)C₁₆alkyl, C(O)C₁₇alkyl, C(O)C₁₈alkyl, C(O)C₁₉alkyl, C(O)C₂₀alkyl, C(O)C₂₁alkyl, C(O)C₆alkyleneCOOH, C(O)C₇alkyleneCOOH, C(O)C₈alkyleneCOOH, C(O)C₉alkyleneCOOH, C(O)C₁₀alkyleneCOOH, C(O)C₁₁alkyleneCOOH, C(O)C₁₂alkyleneCOOH, C(O)C₁₃alkyleneCOOH, C(O)C₁₋₄alkyleneCOOH, C(O)C₁₅alkyleneCOOH, C(O)C₁₆alkyleneCOOH, C(O)C₁₇alkyleneCOOH, C(O)C₁₈alkyleneCOOH, C(O)C₁₉alkyleneCOOH, and C(O)C₂₀alkyleneCOOH. In some embodiments, the albumin binding group is selected from the group consisting of C(O)C₁₋₄alkyleneCOOH, C(O)C₁₆alkyleneCOOH, and C(O)C₁₈alkyleneCOOH.

In some embodiments, the albumin binding group is an unsubstituted or substituted fatty acid moiety. In some embodiments, the fatty acid moiety is a fatty monoacid moiety or a fatty diacid moiety. In some embodiments, the albumin binding group is selected from the group consisting of CH₃—(CH₂)_{M1}—CO— and COOH—(CH₂)_{N1}—CO—, wherein each of M1 and N1 is independently selected from an integer of 6-22. In some embodiments, the albumin binding group is COOH—(CH₂)_{N1}—CO—, wherein N1 is an integer of 6-22. In some embodiments, the albumin binding group is COOH—(CH₂)_{N1}—CO—, wherein N1 is an integer of 6-18. In some embodiments, the albumin binding group is COOH—(CH₂)_{N1}—CO—, wherein N1 is an integer of 10-18. In some embodiments, the albumin binding group is COOH—(CH₂)_{N1}—CO—, wherein N1 is an integer of 6-12. In some embodiments, the albumin binding group is COOH—(CH₂)_{N1}—CO—, wherein N1 is an integer of 14-18. In some embodiments, the albumin binding group is selected from the group consisting of COOH—(CH₂)₆—CO—, COOH—(CH₂)₇—CO—, COOH—(CH₂)₈—CO—, COOH—(CH₂)₉—CO—, COOH—(CH₂)₁₀—CO—, COOH—(CH₂)₁₁—CO—, COOH—(CH₂)₁₂—CO—, COOH—(CH₂)₁₃—CO—, COOH—(CH₂)₁₄—CO—, COOH—(CH₂)₁₅—CO—, COOH—(CH₂)₁₆—CO—, COOH—(CH₂)₁₇—CO—, COOH—(CH₂)₁₈—CO—, COOH—(CH₂)₁₉—CO—, COOH—(CH₂)₂₀—CO—, COOH—(CH₂)₂₁—CO—, and COOH—(CH₂)₂₂—CO—.

In some embodiments, the albumin binding group is COOH—(CH₂)₁₆—CO— or COOH—(CH₂)₁₈—CO.

Additional exemplary albumin binding group that can be used in the present application includes but is not limited to a myristic acid, a substituted or unsubstituted indole-2-carboxylic acid, a substituted or unsubstituted thioamide, a substituted or unsubstituted 4-oxo-4-(5,6,7,8-tetrahydronaphthalen-2-yl)butanoic acid, a substituted or unsubstituted naphthalene acylsulfonamide, a substituted or unsubstituted diphenylcyclohexanol phosphate ester, a substituted or unsubstituted 4-iodophenylalkanoic acid, a substituted or unsubstituted 3-(4-iodophenyl)propionic acid, a substituted or unsubstituted 2-(4-iodophenyl)acetic acid, or a substituted or unsubstituted 4-(4-iodophenyl)butanoic acid.

In some embodiments, the albumin binding group comprises a structure of

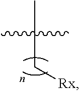

wherein Rx is selected from the group consisting of

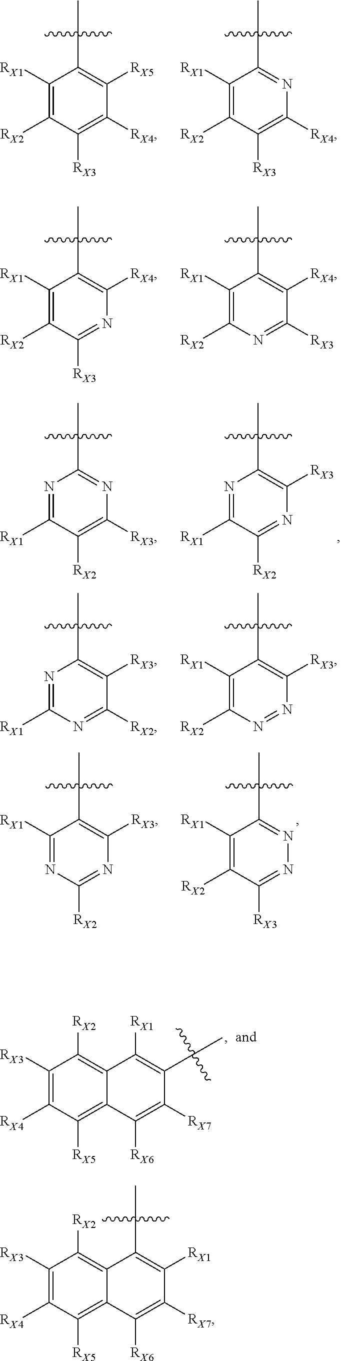

wherein n is any integer ranging from 1 to 6,

each of Rₓ₁, Rₓ₂, Rₓ₃, Rₓ₄, Rₓ₅, Rₓ₆, and Rₓ₇ is independently selected from the group consisting of H, N, S, O, Se, P, halogen, a C₅₋₂₀ aryl group, a C₁₋₂₀ alkyl group, a C₂₋₂₀ alkenyl group, and a C₂₋₂₀ alkynyl group. In some embodiments, the C₅₋₂₀ aryl group, C₁₋₂₀ alkyl group, C₂₋₂₀ alkenyl group or the C₂₋₂₀ alkynyl group can be unsubstituted or substituted by one or more substituents selected from the group consisting of N, S, O, Se, P, and halogen atom. In some embodiments, 0, 1 or 2 carbon atoms of the C₅₋₂₀ aryl group, C₁₋₂₀ alkyl group, C₂₋₂₀ alkenyl group or the C₂₋₂₀ alkynyl group can be replaced by any group selected from the group consisting of C₆₋₁₀ arylene, 5 to 10 membered heteroarylene group, C₃₋₇ carbocyclylen, or 5 to 10 membered heterocyclylene group, and wherein the arylene, heteroarylene, carbocyclylene and heterocyclylene groups are unsubstituted or substituted by one or more substituents selected from the group consisting of N, S, O, Se, P, halogen, C₁₋₆ alkyl, C₁₋₆ alkoxy, C₁₋₆ alkylthiol, —N(C₁₋₆ alkyl)(C₁₋₆ alkyl), nitro and sulfonic acid groups. In some embodiments, 0, 1 or 2 —CH— or —CH₂-groups of the C₅₋₂₀ aryl group, C₁₋₂₀ alkyl group, C₂₋₂₀ alkenyl group or the C₂₋₂₀ alkynyl group can be replaced by any group selected —O—, —S—, —S—S—, —C(O)— or —N(C₁₋₆ alkyl)- group.

In some embodiments, the albumin binding group comprises a structure of

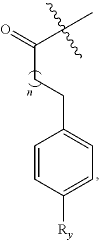

wherein R_{y} is selected from the group consisting of H, F, Cl, Br, I, —CH₃, —OCH₃, COOH, and —CF₃, and n is any integer ranging from 1 to 6.

In some embodiments, the albumin binding group is selected from the group consisting of

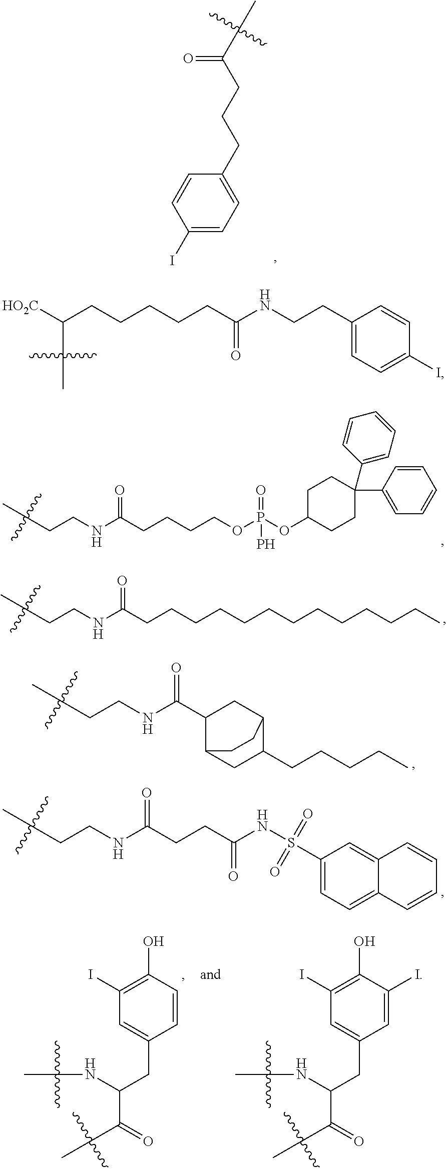

and

In some embodiments, A comprises a structure of formula (I-Dual-A):

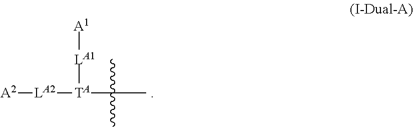

wherein each of A¹ and A² is independently a potency enhancing moiety, A¹ is conjugated to T^{A} via a linker L^{A1}, A² is conjugated to T via a linker L^{A2}, T^{A} is conjugated to T via the linker L^{A} as described herein.

In some embodiments, each of A¹ and A² is independently an albumin binding group as described herein. In some embodiments, A¹ and A² are identical. In some embodiments, A¹ and A² are different.

In some embodiments, each of A¹ and A² is independently selected from the group consisting of unsubstituted or substituted C(O)C₁₋₂₆alkyleneCOOH, unsubstituted or substituted C(O)C₁₋₂₆alkenylenerOOH, unsubstituted or substituted C(O)C₁₋₂₆alkyl, and unsubstituted or substituted C(O)C₁₋₂₆alkenyl. In some embodiments, each of A¹ and A² is independently selected from the group consisting of unsubstituted or substituted C(O)C₆₋₂₀alkyleneCOOH, and unsubstituted or substituted C(O)C₇₋₂₁alkyl. In some embodiments, each of A¹ and A² is independently selected from the group consisting of C(O)C₇alkyl, C(O)C₈alkyl, C(O)C₉alkyl, C(O)C₁₀alkyl, C(O)C₁₁alkyl, C(O)C₁₂alkyl, C(O)C₁₃alkyl, C(O)C₁₋₄alkyl, C(O)C₁₅alkyl, C(O)C₁₆alkyl, C(O)C₁₇alkyl, C(O)C₁₈alkyl, C(O)C₁₉alkyl, C(O)C₂₀alkyl, C(O)C₂₁alkyl, C(O)C₆alkyleneCOOH, C(O)C₇alkyleneCOOH, C(O)C₈alkyleneCOOH, C(O)C₉alkyleneCOOH, C(O)C₁₀alkyleneCOOH, C(O)C₁₁alkyleneCOOH, C(O)C₁₂alkyleneCOOH, C(O)C₁₃alkyleneCOOH, C(O)C₁₋₄alkyleneCOOH, C(O)C₁₅alkyleneCOOH, C(O)C₁₆alkyleneCOOH, C(O)C₁₇alkyleneCOOH, C(O)C₁₈alkyleneCOOH, C(O)C₁₉alkyleneCOOH, and C(O)C₂₀alkyleneCOOH. In some embodiments, each of A¹ and A² is independently selected from the group consisting of C(O)C₁₋₄alkyleneCOOH, C(O)C₁₆alkyleneCOOH, and C(O)C₁₈alkyleneCOOH.

In some embodiments, each of A¹ and A² is independently a fatty acid moiety. In some embodiments, the fatty acid moiety is a fatty monoacid moiety or a fatty diacid moiety. In some embodiments, each of A¹ and A² is independently selected from the group consisting of CH₃—(CH₂)_{M1}—CO— and COOH—(CH₂)_{N1}—CO—, wherein each of M1 and N1 is independently selected from an integer of 6-22. In some embodiments, A¹ is COOH—(CH₂)_{N1}—CO— and A² is CH₃—(CH₂)_{M1}—CO—, wherein each of M1 and N1 is independently selected from an integer of 6-22. In some embodiments, A² is COOH—(CH₂)_{N1}—CO— and A¹ is CH₃—(CH₂)_{M1}—CO—, wherein each of M1 and N1 is independently selected from an integer of 6-22. In some embodiments, A¹ is COOH—(CH₂)₁₆—CO— and A² is CH₃—(CH₂)_{M1}—CO—, wherein M1 is an integer of 10-16. In some embodiments, A² is COOH—(CH₂)₁₆—CO— and A¹ is CH₃—(CH₂)_{M1}—CO—, wherein M1 is an integer of 10-16. In some embodiments, A¹ is COOH—(CH₂)₁₈—CO— and A² is CH₃—(CH₂)_{M1}—CO—, wherein M1 is an integer of 10-16. In some embodiments, A² is COOH—(CH₂)₁₈—CO— and A¹ is CH₃—(CH₂)_{M1}—CO—, wherein M1 is an integer of 10-16.

In some embodiments, A¹ is COOH—(CH₂)₁₆—CO— and A² is CH₃—(CH₂)₁₀—CO—. In some embodiments, A² is COOH—(CH₂)₁₆—CO— and A¹ is CH₃—(CH₂)₁₀—CO—. In some embodiments, A¹ is COOH—(CH₂)₁₆—CO— and A² is CH₃—(CH₂)₁₂—CO—. In some embodiments, A² is COOH—(CH₂)₁₆—CO— and A¹ is CH₃—(CH₂)₁₂—CO—. In some embodiments, A¹ is COOH—(CH₂)₁₆—CO— and A² is CH₃—(CH₂)₁₄—CO—. In some embodiments, A² is COOH—(CH₂)₁₆—CO— and A¹ is CH₃—(CH₂)₁₄—CO—. In some embodiments, A¹ is COOH—(CH₂)₁₆—CO— and A² is CH₃—(CH₂)₁₆—CO—. In some embodiments, A² is COOH—(CH₂)₁₆—CO— and A¹ is CH₃—(CH₂)₁₆—CO—. In some embodiments, A¹ is COOH—(CH₂)₁₈—CO—, and A² is CH₃—(CH₂)₁₀—CO—. In some embodiments, A² is COOH—(CH₂)₁₈—CO—, and A¹ is CH₃—(CH₂)₁₀—CO—. In some embodiments, A¹ is COOH—(CH₂)₁₈—CO— and A² is CH₃—(CH₂)₁₂—CO—. In some embodiments, A² is COOH—(CH₂)₁₈—CO— and A¹ is CH₃—(CH₂)₁₂—CO—. In some embodiments, A¹ is COOH—(CH₂)₁₈—CO— and A² is CH₃—(CH₂)₁₄—CO—. In some embodiments, A² is COOH—(CH₂)₁₈—CO— and A¹ is CH₃—(CH₂)₁₄—CO—. In some embodiments, A¹ is COOH—(CH₂)₁₈—CO— and A² is CH₃—(CH₂)₁₆—CO—. In some embodiments, A² is COOH—(CH₂)₁₈—CO— and A¹ is CH₃—(CH₂)₁₆—CO—.

In some embodiments, each of A¹ and A² is independently COOH—(CH₂)_{N1}—CO—, wherein N1 is an integer of 6-18. In some embodiments, each of A¹ and A² is independently COOH—(CH₂)_{N1}—CO—, wherein N1 is an integer of 10-18. In some embodiments, each of A¹ and A² is independently COOH—(CH₂)_{N1}—CO—, wherein N1 is an integer of 14-18.

In some embodiments, A¹ is COOH—(CH₂)₁₂—CO— and A² is COOH—(CH₂)₁₂—CO—. In some embodiments, A¹ is COOH—(CH₂)₁₄—CO— and A² is COOH—(CH₂)₁₄—CO—. In some embodiments, A¹ is COOH—(CH₂)₁₆—CO— and A² is COOH—(CH₂)₁₆—CO—. In some embodiments, A¹ is COOH—(CH₂)₁₈—CO— and A² is COOH—(CH₂)₁₈—CO—. In some embodiments, A¹ is COOH—(CH₂)₁₆—CO— and A² is COOH—(CH₂)₁₀—CO—. In some embodiments, A² is COOH—(CH₂)₁₆—CO— and A¹ is COOH—(CH₂)₁₀—CO—. In some embodiments, A¹ is COOH—(CH₂)₁₆—CO— and A² is COOH—(CH₂)₁₂—CO—. In some embodiments, A² is COOH—(CH₂)₁₆—CO— and A¹ is COOH—(CH₂)₁₂—CO—. In some embodiments, A¹ is COOH—(CH₂)₁₆—CO— and A² is COOH—(CH₂)₁₄—CO—. In some embodiments, A² is COOH—(CH₂)₁₆—CO— and A¹ is COOH—(CH₂)₁₄—CO—. In some embodiments, A¹ is COOH—(CH₂)₁₆—CO— and A² is COOH—(CH₂)₁₈—CO—. In some embodiments, A² is COOH—(CH₂)₁₆—CO— and A¹ is COOH—(CH₂)₁₈—CO—.

In some embodiments, each of L^{A1} and L^{A2} is independently a direct bond or a divalent linker. In some embodiments, L^{A1} and L^{A2} are both direct bonds. In some embodiments, L^{A1} is a direct bond and L^{A2} a divalent linker. In some embodiments, L^{A2} is a direct bond and L^{A1} a divalent linker. In some embodiments, L^{A1} and L^{A2} are both divalent linkers.

In some embodiments, at least one of L^{A1} and L^{A2} is a divalent linker, and the divalent linker comprises one or more groups selected from the group consisting of R¹NC₁₋₂₀ alkyleneNR², R¹NC₁₋₂₀alkenyleneNR² C(O)C₁₋₂₀alkyleneC(O), C(O)C₁₋₂₀alkenyleneC(O), R¹NC₁₋₂₀alkyleneC(O), R¹NC₁₋₂₀alkenyleneC(O), C(O)C₁₋₂₀alkyleneNHR², C(O)C₁₋₂₀alkenyleneNR², C(S)C₁₋₂₀alkyleneC(S), C(S)C₁₋₂₀alkenyleneC(S), C(S)C₁₋₂₀alkyleneC(O), C(S)C₁₋₂₀alkenyleneC(O), C(O)C₁₋₂₀alkyleneC(S), C(O)C₁₋₂₀alkenyleneC(S), C(O)C₁₋₂₀alkyleneO, C(O)C₁₋₂₀alkenyleneO, R¹NC₁₋₂₀alkenyleneC(S), C(S)C₁₋₂₀alkyleneNR² or C(S)C₁₋₂₀alkenyleneNR², each of which is independently and optionally interrupted by one or more of S, O, NH, N(C₁₋₆alkyl), C(O), C(O)NH, NHC(O), C(S)NH, NHC(S), NHC(O)NH, NHC(S)NH, NHC(NH), NHC(NC₁₋₄alkyl), C(NH)NH, C(NC₁₋₄alkyl)NH, NC₄₋₁₀cycloalkyl, C₄₋₁₀heterocycloalkyl, C₆₋₁₀aryl and C₅₋₁₀heteroaryl, and each alkyl, alkylene and alkenylene is optionally and independently substituted with one or more substituents selected from the group consisting of halo, COOH, C₁₋₆alkyl, OH, OC₁₋₆alkyl, SH, SC₁₋₆alkyl, NR³R⁴, C₁₋₄alkyleneOH, C₁₋₄alkyleneOC₁₋₄alkyl and C₁₋₄alkyleneNR³R⁴, wherein each R¹, R², R³ and R⁴ is independently selected from the group consisting of H and C₁₋₄alkyl.

In some embodiments, at least one of L^{A1} and L^{A2} is a divalent linker, and the divalent linker comprises one or more amino acid residues. In some embodiments, the amino acid residue of the divalent linker is derived from a naturally occurring amino acid selected from the group consisting of alanine (Ala, A), arginine (Arg, R), asparagine (Asn, N), aspartic acid (Asp, D), glutamine (Gln, Q), glutamic acid (Glu, E), γGlu, glutamine (Gln), glycine (Gly, G), histidine (His, H), isoleucine (Ile, I), leucine (Leu, L), Lysine (Lys, K), εLys, methionine (Met, M), phenylalanine (Phe, F), proline (Pro, P), serine (Ser, S), threonine (Thr, T), tryptophan (Trp, W), tyrosine (Tyr, Y), valine (Val, V), pyrrolysine (Pyl, O), pyrroline-carboxy-lysine (PCL), gamma-carboxyglutamic acid (Gla), and a D enantiomer thereof. In some embodiments, the amino acid residue of the divalent linker is derived from a unnaturally occurring amino acid selected from the group consisting of NR⁵C₅alkyleneC(O) (Ahx), L-Cysteic acid (Cya), norleucine (Nle), norvaline (Nva), 2-aminooctanoic acid (Aoc), 2-naphthylalanine (2-Nal), 3-(trifluoromethyl)phenylalanine (TFP), homophenylalanine (hPhe), cyclohexylalanine (Cha), 1-naphthylalanine (1-Nal), 4-benzoyl-L-phenylalanine (Bpa), 2-methoxy-4-vinylphenylalanine (MvF), 4-fluorophenylalanine (4-F-Phe), 4-phenyl-2,3-dihydroxy-6-nitrophenylalanine (pNIPA), 2-(2-naphthyl)alanine (2-Nal-ala), 4-(4-propoxyphenyl)alanine (Ppa), 4-carboxyphenylalanine (4-CPA), 4-butylphenylalanine (Bua), 2-nitrophenylalanine (2-Npa), 4-azidophenylalanine (4-AzF), 2-(4-nitrophenyl)ethylalanine (2-Npe), 3-iodo-L-tyrosine (Ity), 5,5,5-trifluoroleucine (TFL), and a D enantiomer thereof.

In some embodiments, at least one of L^{A1} and L^{A2} is a divalent linker, and the divalent linker comprises —N(R^{N})(C₂alkyleneO)ₓ₁X^{b}C(O), wherein R^{N} is H or C₁-C₆ alkyl, x1 is an integer from 1 to 10, and X^{b} is C₁-C₃ alkylene. In some embodiments, the —N(R^{N})(C₂alkyleneX^{a})ₓ₁X^{b}C(O)— is selected from the group consisting of —N(R^{N})(C₂alkyleneO)₂C₁alkyleneC(O), —N(R^{N})(C₂alkyleneO)C₂alkyleneC(O), —N(R^{N})(C₂alkyleneO)₃C₂alkyleneC(O), and —N(R^{N})(C₂alkyleneO)₆C₂alkyleneC(O), wherein R^{N} is H or C₁-C₆ alkyl. In some embodiments, R^{N} is H. In some embodiments, R^{N} is methyl, ethyl, or propyl.

In some embodiments, at least one of L^{A1} and L^{A2} is a divalent linker, and the divalent linker comprises one or more moieties —(OCH₂CH₂)ₙ—, and n is an integer of 1-28. In some embodiments, at least one of L^{A1} and L^{A2} is a divalent linker, and the divalent linker comprises one or more groups selected from the group consisting of —(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), and —(C₂alkyleneO)₆C₂alkyleneC(O) (PEG6), —(C₂alkyleneO)₆C₂alkyleneC(O) (PEG8), and —(C₂alkyleneO)₆C₂alkyleneC(O) (PEG12).

In some embodiments, at least one of L^{A1} and L^{A2} is a cleavable linker as described herein.

In some embodiments, the compound of the present application comprises an effector moiety E. In some embodiments, E is a chemotherapeutic agent, a toxin, an immunomodulator, a diagnostic agent, a radionuclide, or a chelating group.

In some embodiments, E of the compound comprises a chemotherapeutic agent capable of killing a tumor cell. Examples of chemotherapeutic agents include but are not limited to Erlotinib (TARCEVA®, Genentech/OSI Pharm.), Bortezomib (VELCADE®, Millennium Pharm.), Fulvestrant (FASLODEX®, Astrazeneca), Sutent (SUl 1248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (GSK572016, GlaxoSmithKline), Lonafarnib (SCH 66336), Sorafenib (BAY43-9006, Bayer Labs.), and Gefitinib (IRESSA®, Astrazeneca), AG1478, AG1571 (SU 5271; Sugen), alkylating agents such as Thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins; a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall; dynemicin, including dynemicin A; bisphosphonates, such as clodronate; an esperamicin; as well as neocarzinostatin chromophore and related chromoprotein enediyne antibiotic chromophores), aclacinomysins, actinomycin, anthramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N. J.), ABRAXANE™ Cremophor-free, albumin-engineered nanoparticle formulation of paclitaxel (American Pharmaceutical Partners, Schaumberg, Illinois), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluorometlhylornithine (DMFO); retinoids such as retinoic acid; capecitabine; and pharmaceutically acceptable salts, acids or derivatives of any of the above.

In some embodiments, the chemotherapeutic agent also includes but is not limited to (i) anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON-toremifene; (ii) aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; (iii) anti-androgens such as flutamide, nilutamide, bicalutamide, leuprolide, and goserelin; as well as troxacitabine (a 1,3-dioxolane nucleoside cytosine analog); (iv) aromatase inhibitors; (v) protein kinase inhibitors; (vi) lipid kinase inhibitors; (vii) antisense oligonucleotides, particularly those which inhibit expression of genes in signaling pathways implicated in abherant cell proliferation, such as, for example, PKC-alpha, Ralf and H-Ras; (viii) ribozymes such as a VEGF expression inhibitor (e.g., ANGIOZYME® ribozyme) and a HER2 expression inhibitor; (ix) vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; (x) anti-angiogenic agents such as bevacizumab (AVASTIN®, Genentech); and (xi) pharmaceutically acceptable salts, acids or derivatives of any of the above.

In some embodiments, E of the compound as described herein comprises a cytokine. The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-a and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-a and TGF-β; insulin-like growth factor-I and —II; erythropoietin (EPO); osteoinductive factors; interferons such as interferon-α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1a, IL-2, IL-3, IL-4, IL-5, IL-6, IL-7, IL-8, IL-9, IL-10, IL-11, IL-12; a tumor necrosis factor such as TNF-α or TNF-β; and other polypeptide factors including LIF and kit ligand (KL).

In some embodiments, E of the compound as described herein comprises a diagnostic agent. In some embodiments, the diagnostic agent is a label selected from the group consisting of a fluorescent label, a chromogenic label, or a radiolabel. Therefore, in some embodiments, the diagnostic agent is selected from the group consisting of a fluorescent compound, an enzyme, a prosthetic group, a luminescent material, a bioluminescent material, or a radioactive material.

In some embodiments, E comprises a radionuclide. In some embodiments, the radionuclide is radioactive isotope of H, C, N, F, Br, I, S, P and At. In some embodiments, the radionuclide is selected from the group consisting of ¹¹C, ¹³N, ¹⁴C, ¹⁵O, ¹⁸F, ⁷⁵Br, ⁷⁶Br, ⁷⁷Br, ¹²³I, ¹²⁴I, ¹²⁵I, ¹³¹I, ³⁵S, ²¹¹At ³²P, ³³P, ⁷¹As, ⁷²As, ⁷⁴As, ⁷⁶As, and ⁷⁷As. In some embodiments, E comprises a radionuclide selected from the group consisting of ¹⁸F, ¹²⁵I, ¹³¹I, and ²¹¹At.

In some embodiment, E of the compound as described herein comprises a chelating group that is capable of binding with and/or complexing a metal ion. In some embodiments, E comprises a chelating group that is capable of binding with and/or complexing a metal ion to form a heterocyclic ring including the metal ion. Any suitable chelating group known in the art can be used in E of the compound, for example, as disclosed in Banerjee et al., Nucl. Med. Biol., 2005, 32, 1-20, Wadas et al., Chem. Rev., 2010, 110, 2858-2902, U.S. Pat. Nos. 5,367,080, 5,364,613, 5,021,556, 5,075,099, and 5,886,142.

A person skilled in the art would appreciate that “a chelating group derived from a chelating agent” as used herein refers to a chelating agent derivative formed after the chelating agent is connected to the conjugate of Formula I. For example, “a chelating group derived from a chelating agent” may be a chelating agent without the “—OH” (or ester thereof) of an available carboxyl group (or ester thereof) on the chelating agent, without the “H” portion of an available amino group on the chelating agent, without the “NCS” portion of an available isothiocyanate on the chelating agent, without the “H” portion of an available maleimide group on the chelating agent, a chelating agent after an available acetylene group on the chelating agent has been reacted to connect to the conjugate of Formula I, or a chelating agent after an available tetrazole group on the chelating agent has been reacted to connect to the conjugate of Formula I. For example, a person skilled in the art would appreciate that when E is a chelating group derived from a DOTA, one “—OH” from one of the four available carboxyl groups on DOTA is removed to form the connection to L^{A} (or T when L^{A} is a direct bond) in the conjugate of Formula I.

In some embodiments, E comprises a chelating group derived from a chelating agent. In some embodiments, the chelating agent is selected from the group consisting of a cyclic and an acyclic bifunctional chelating agent capable of complexing one or more radionuclides. In some embodiments, the chelating agent is any one selected from the group consisting of 1,4,7-Triazacyclononane (TACN); 1,4,7-triazacyclononane-triacetic acid (NOTA); 1,4,7-triazacyclononane-N-succinic acid-N′,N″-diacetic acid (NOTASA); 1,4,7-triazacyclononane-N-glutamic acid-N′,N″-diacetic acid (NODAGA); 1,4,7-triazacyclononane-N,N′,N″-tris(methylenephosphonic) acid (NOTP); 1,4,7,10-tetraazacyclododecane ([12]aneN4) (cyclen); 1,4,7,10-tetraazacyclotridecane ([13]aneN4); 1,4,7,11-tetraazacyclotetradecane (iso-cyclam); 1,4,7,10-tetraazacyclododecane-1,4,7,10-tetraacetic acid (DOTA); 2-(1,4,7,10-tetraazacyclododecan-1-yl)acetate (DO1A); 2,2′-(1,4,7,10-tetraazacyclododecane-1,7-diyl)diacetic acid (DO2A); 2,2′,2″-(1,4,7,10-tetraazacyclododecane-1 ,4,7-triyl)triacetic acid (DO3A); 1,4,7,10-tetraazacyclododecane-1,4,7,10-tetra(methanepnosphonic acid) (DOTP); 1,4,7,10-tetraazacyclododecane-1,7-di(methanephosphoriic acid) (DO2P); 1,4,7,10-tetraazacyclododecane-1,4,7-tri(methanephosphonic acid) (DO3P); 1,4,7,10-tetraazacyclo-decane-1-glutamic acid-4,7,10-triacetic acid (DOTAGA); 1,4,7,10-tetraazacyclodecane-1-succinic acid-4,7,10-triacetic acid (DOTASA); 1,4,8,11-tetraazacyclotetradecane ([14]aneN4) (cyclam); 1,4,8,12-tetraazacyclopentadecane ([15]aneN4); 1,5,9,13-tetraazacyclohexadecane ([16]aneN4); 1,4-ethano-1,4,8,11-tetraazacyclo-tetradecane (et-cyclam); 1,4,8,11-tetraazacyclotetradecane-1,4,8,1 1-tetraacetic acid (TETA); 2-(1,4,8,11-tetraazacyclotetradecane-1-y I) acetic acid (TE1A); 2,2′-(1,4,8,11-tetraazacyclotetradecane-1,8-diyl)diacetic acid (TE2A); 4,11-bis(carboxy methyl)-1,4,8,11-tetraazabicyclo[6.6.2]-hexadecane (CB-TE2A); 3,6,10,13,16,19-hexaazabicyclo[6.6.6]icosane (Sar); 1,4,7,10-tetra-(2-carbamoyl-methyl)-cyclododecane (TCMC); N,N′-bis[(6-carboxy-2-pyridil)methyl]-4,13-diaza-18-crown-6 (macropa), phthalocyanines; porphyrins; PCTA (3,6,9,15-tetraazabicyclo[9.3.1]pentadeca-1(15),11,13-triene-3,6,9-triacetic acid); DEPA (7-[2-(biscarboxymethylamino)ethyl]-4,10-biscarboxymethyl-1,4,7,10-tetraazacyclododec-1-yl-acetic acid); DTPA (1,1,4,7,7-diethylenetriaminepentaacetic acid); CHX-DTPA (cyclohexane-1,2-diamineN,N,N′,N′-tetraacetate); BATPA (1,2-bis[2-aminophenoxy]ethane-N,N,N′,N′-tetraacetic acid); TTHA (triethylenetetramineN,N,N′,N″,N″′,N″′-hexaacetic acid); HBED (N,N′-bis[2-hydroxybenzyl]ethylenediamine-N,N′-diacetic acid); EGTA (ethylene glycol bis[2-aminoethyl ether]-N,N,N′,N′-tetraacetic acid); EDTMP (ethylenediamine tetra-[methylene phosphonic acid]); TRAP (triazacyclononate phosphinic acids); SHBED (N,N′-bis[2-hydroxy-5-sulfobenzyl]ethylenediaminediacetic acid); H6Sbbpen(N,N′-bis-[2-hydroxy-5-sulfonylbenzyl]-N,N′-bis[2-methylpyridyl]ethylenediamine); THP (Tris(3,4-hydroxypyridinone); DFO (deferoxamine); FSC (Fusarinine); TAFC (triacetylfusarinine C); FOXE (ferrioxamine E); 6SS (N,N′-bis[2,2-dimethyl-2-mercaptoethyl]ethylenediamine-N,N′-diacetic acid); ECC (ethylenecysteamine cysteine); ECD (ethyl cysteinate dimer); NETA ([2-{4,7-biscarboxymethyl(1,4,7)triazacyclonona-1-yl-ethyl}carbonylmethylamino]acetic acid; THPN (Tetrakis(3-Hydroxy-4-Pyridinone)); H2dedpa (1,2-[{6-(carboxylato-)pyridin-2-yl}methylamino]-ethane); H4octapa (N,N′-bis[6-carboxy-2-pyridylmethyl]-ethylenediamine-N,N′-diacetic acid); H2bispa2 (6,6′-[{9-hydroxy-1,5-bis-(methoxycarbonyl)-2,4-di(pyridin-2-yl)-3,7-diazabicyclo[3.3.1]nonane-3,7-diyl}bis(methylene)]dipicolinic acid); DOTMP(1,4,7,10-Tetraazacyclododecane-1,4,7,10-tetrayl-tetrakis(methylphosphonic acid)); PEPA (1,4,7,10,13-pentaazocyclopentadecane pentaacetic acid); HEHA (1,4,7,10,13,16-hexaazocyclooctadecane hexaacetic acid); H2hox; H2CHXhox; H2octox; H2pyhox; H4neunopa; TETPA; H4pypa; H4py4pa; DTPAm; EGTAm; ampam; Me-3,2-HOPO; 3,4,3-(L1-1,2-HOPO); macrocyclic tetrapthalimide; and any derivatives thereof. In some embodiments, E comprises a chelating group derived from a chelating agent selected from the group consisting of DOTA and DOTAGA.

In some embodiments, E is connected to L^{E} or T through any suitable functional groups. In some embodiments, E is a chelating group comprising two or more carboxyl groups, and E is connected to L^{E} or T through a carboxyl functional group. In some embodiments, E is a chelating group derived from DOTA or DOTAGA and is connected to L^{E} or T through any one of the available carboxyl functional groups. In some embodiments, E is a chelating group derived from DOTA and is connected to L^{E} or T through any one of the available carboxyl functional groups.

In some embodiments, E comprises a chelating group complexed with a radionuclide. In some embodiments, E comprises a chelating group complexed with a radioactive isotope selected from the group consisting of As, Se, K, Sc, Ti, Cr, Mn, Fe, Co, Ni, Cu, Ga, Ge, Rb, Sr, Y, Zr, Nb, Tc, Rh, Pd, In, Sn, Sb, Zn, Ta, W, Re, Os, Ir, Pt, Au, Hg, Ti, Pb, Bi, Po, Fr, Pm, a lanthanide, an actinide, Mg, Al, Ca, Cd, and Ba. In some embodiments, the lanthanide is Lu, Sm, Ho, or Tb. In some embodiments, the actinide is Ac, Th, or U. In some embodiments, the one or more radionuclides are each independently selected from the group consisting of ⁹⁹Tc, ^{99m}Tc, ¹⁸⁸Re, ¹⁸⁶Re, ¹⁵³Sm, ⁶⁶Ga ⁶⁷Ga, ⁶⁸Ga, ¹¹¹n, ¹²³In ⁵⁹Fe, ⁶³Zn, ⁵²Fe, ⁵²Mn, ⁴⁵Ti ⁶⁰Cu, ⁶¹Cu, ⁶⁷Cu, ⁶⁴Cu, ⁶²Cu, ⁸²Rb, ^{195m}Pt, ^{191m}Pt, ^{193m}Pt, ^{117m}Sn, ⁸⁹Zr, ¹⁷⁷Lu, ¹⁸F, ¹⁸⁸Re, ¹⁸⁶Re, ¹⁵³Sm, ⁶⁶Ho, ⁸⁶Y, ⁸⁷Y, ⁹⁰Y, ⁸⁹Sr, ¹⁵³Gd, ¹⁵⁹Gd, ²²⁵Ac, ²¹²Bi, ²¹³Bi, ¹⁹⁸Au, ¹⁹⁹Au, ^{193m}Pt, ¹⁹⁷Pt, ¹⁰³Pd, ¹⁰⁹Pd, ¹⁰⁵Rh, ^{101m}Rh, ^{103m}Rh, ²²³Ra, ²²⁴Ra, ⁹⁷Ru, ²²⁷Th, ²²⁹Th, ¹⁶¹Tb, ¹⁴⁹Tb, ²⁰³Pb, ²¹²Pb, ²⁰¹TI, ¹¹⁹Sb, ^{58m}Co, Co, ⁵⁷Co, ⁴⁷Sc, ¹⁴⁹Pm, ¹⁴²Pr, ¹⁶¹Ho, ¹⁶⁶Ho, ¹⁷⁵Yb, and ⁵¹Cr.

In some embodiments, A of the compound is conjugated to T via a linker L^{A} and E of the compound is conjugated to T via a linker L^{E}. In some embodiments, each of L^{E} and L^{A} of the compound as described herein is independently a direct bond or a divalent linker.

In some embodiments, L^{E} and L^{A} are both direct bonds. In some embodiments, L^{E} is a direct bond and L^{A} a divalent linker. In some embodiments, L^{A} is a direct bond and L^{E} a divalent linker. In some embodiments, L^{E} and L^{A} are both divalent linkers.

In some embodiments, at least one of L^{E} and L^{A} is a divalent linker, and the divalent linker comprises one or more groups selected from the group consisting of R¹NC₁₋₂O alkyleneNR², R¹NC₁₋₂₀alkenyleneNR² C(O)C₁₋₂₀alkyleneC(O), C(O)C₁₋₂₀alkenyleneC(O), R¹NC₁₋₂₀alkyleneC(O), R¹NC₁₋₂₀alkenyleneC(O), C(O)C₁₋₂₀alkyleneNHR², C(O)C₁₋₂₀alkenyleneNR², C(S)C₁₋₂₀alkyleneC(S), C(S)C₁₋₂₀alkenyleneC(S), C(S)C₁₋₂₀alkyleneC(O), C(S)C₁₋₂₀alkenyleneC(O), C(O)C₁₋₂₀alkyleneC(S), C(O)C₁₋₂₀alkenyleneC(S), C(O)C₁₋₂₀alkyleneO, C(O)C₁₋₂₀alkenyleneO, R¹NC₁₋₂₀alkenyleneC(S), C(S)C₁₋₂₀alkyleneNR² and C(S)C₁₋₂₀alkenyleneNR², each of which is independently and optionally interrupted by one or more of S, O, NH, N(C₁₋₆alkyl), C(O), C(O)NH, NHC(O), C(S)NH, NHC(S), NHC(O)NH, NHC(S)NH, NHC(NH), NHC(NC₁₋₄alkyl), C(NH)NH, C(NC₁₋₄alkyl)NH, NC₄₋₁₀cycloalkyl, C₄₋₁₀heterocycloalkyl, C₆₋₁₀aryl and C₅₋₁₀heteroaryl, and each alkyl, alkylene and alkenylene is optionally and independently substituted with one or more substituents selected from the group consisting of halo, COOH, C₁₋₆alkyl, OH, OC₁₋₆alkyl, SH, SC₁₋₆alkyl, NR³R⁴, C₁₋₄alkyleneOH, C₁₋₄alkyleneOC₁₋₄alkyl and C₁₋₄alkyleneNR³R⁴, wherein each R¹, R², R³ and R⁴ is independently selected from the group consisting of H and C₁₋₄alkyl.

In some embodiments, at least one of L^{E} and L^{A} is a divalent linker, and the divalent linker comprises one or more amino acid residues. In some embodiments, the amino acid residue of the divalent linker is derived from a naturally occurring amino acid selected from the group consisting of alanine (Ala, A), arginine (Arg, R), asparagine (Asn, N), aspartic acid (Asp, D), glutamine (Gln, Q), glutamic acid (Glu, E), γGlu, glutamine (Gln), glycine (Gly, G), histidine (His, H), isoleucine (Ile, I), leucine (Leu, L), Lysine (Lys, K), εLys, methionine (Met, M), phenylalanine (Phe, F), proline (Pro, P), serine (Ser, S), threonine (Thr, T), tryptophan (Trp, W), tyrosine (Tyr, Y), valine (Val, V), pyrrolysine (Pyl, O), pyrroline-carboxy-lysine (PCL), gamma-carboxyglutamic acid (Gla), and a D enantiomer thereof. In some embodiments, the amino acid residue of the divalent linker is derived from a unnaturally occurring amino acid selected from the group consisting of NR⁵C₅alkyleneC(O) (Ahx), L-Cysteic acid (Cya), norleucine (Nle), norvaline (Nva), 2-aminooctanoic acid (Aoc), 2-naphthylalanine (2-Nal), 3-(trifluoromethyl)phenylalanine (TFP), homophenylalanine (hPhe), cyclohexylalanine (Cha), 1-naphthylalanine (1-Nal), 4-benzoyl-L-phenylalanine (Bpa), 2-methoxy-4-vinylphenylalanine (MvF), 4-fluorophenylalanine (4-F-Phe), 4-phenyl-2,3-dihydroxy-6-nitrophenylalanine (pNIPA), 2-(2-naphthyl)alanine (2-Nal-ala), 4-(4-propoxyphenyl)alanine (Ppa), 4-carboxyphenylalanine (4-CPA), 4-butylphenylalanine (Bua), 2-nitrophenylalanine (2-Npa), 4-azidophenylalanine (4-AzF), 2-(4-nitrophenyl)ethylalanine (2-Npe), 3-iodo-L-tyrosine (Ity), or 5,5,5-trifluoroleucine (TFL), and a D enantiomer thereof.

In some embodiments, at least one of L^{E} and L^{A} is a divalent linker, and the divalent linker comprises —N(R^{N})(C₂alkyleneO)ₓ₁X^{b}C(O), wherein R^{N} is H or C₁-C₆ alkyl, x1 is an integer from 1 to 10, and X^{b} is C₁-C₃ alkylene. In some embodiments, the —N(R^{N})(C₂alkyleneX^{a})ₓ₁X^{b}C(O)— is selected from the group consisting of —N(R^{N})(C₂alkyleneO)₂C₁alkyleneC(O), —N(R^{N})(C₂alkyleneO)C₂alkyleneC(O), —N(R^{N})(C₂alkyleneO)₃C₂alkyleneC(O), and —N(R^{N})(C₂alkyleneO)₆C₂alkyleneC(O), wherein R^{N} is H or C₁-C₆ alkyl. In some embodiments, R^{N} is H. In some embodiments, R^{N} is methyl, ethyl, or propyl.

In some embodiments, the divalent linker comprises one or more moieties of —(OCH₂CH₂)ₙ—, wherein n is an integer of 1-28. In some embodiments, the divalent linker comprises one or more moieties each independently selected from the group consisting of —(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), and —(C₂alkyleneO)₆C₂alkyleneC(O) (PEG6), —(C₂alkyleneO)₆C₂alkyleneC(O) (PEG8), and —(C₂alkyleneO)₆C₂alkyleneC(O) (PEG12).

In some embodiments, at least one of L^{E} and L^{A} is a cleavable linker. In some embodiments, L^{E} and L^{A} are both cleavable linkers. In some embodiments, L^{E} is a cleavable linker and L^{A} is a non-cleavable linker. In some embodiments, L^{A} is a cleavable linker and L^{E} is a non-cleavable linker.

In some embodiments, the cleavable linker comprises one or more amino acid residues. In some embodiments, the amino acid residue of the cleavable linker is derived from a naturally occurring amino acid selected from the group consisting of alanine (Ala, A), arginine (Arg, R), asparagine (Asn, N), aspartic acid (Asp, D), glutamine (Gln, Q), glutamic acid (Glu, E), γGlu, glutamine (Gln), glycine (Gly, G), histidine (His, H), isoleucine (Ile, I), leucine (Leu, L), Lysine (Lys, K), εLys, methionine (Met, M), phenylalanine (Phe, F), proline (Pro, P), serine (Ser, S), threonine (Thr, T), tryptophan (Trp, W), tyrosine (Tyr, Y), valine (Val, V), pyrrolysine (Pyl, O), pyrroline-carboxy-lysine (PCL), gamma-carboxyglutamic acid (Gla), and a D enantiomer thereof. In some embodiments, the amino acid residue of the cleavable linker is derived from a unnaturally occurring amino acid selected from the group consisting of NR⁵C₅alkyleneC(O) (Ahx), L-Cysteic acid (Cya), norleucine (Nle), norvaline (Nva), 2-aminooctanoic acid (Aoc), 2-naphthylalanine (2-Nal), 3-(trifluoromethyl)phenylalanine (TFP), homophenylalanine (hPhe), cyclohexylalanine (Cha), 1-naphthylalanine (1-Nal), 4-benzoyl-L-phenylalanine (Bpa), 2-methoxy-4-vinylphenylalanine (MvF), 4-fluorophenylalanine (4-F-Phe), 4-phenyl-2,3-dihydroxy-6-nitrophenylalanine (pNIPA), 2-(2-naphthyl)alanine (2-Nal-ala), 4-(4-propoxyphenyl)alanine (Ppa), 4-carboxyphenylalanine (4-CPA), 4-butylphenylalanine (Bua), 2-nitrophenylalanine (2-Npa), 4-azidophenylalanine (4-AzF), 2-(4-nitrophenyl)ethylalanine (2-Npe), 3-iodo-L-tyrosine (Ity), 5,5,5-trifluoroleucine (TFL), and a D enantiomer thereof.

In some embodiments, the cleavable linker comprises one or more cleavable moieties selected from the group consisting of S—S, C(O)O, OC(O), OC(O)O, OC(O)NH, NHC(O)O, SC(O), C(O)S, C═NNH, C═NO, NC₁₋₄alkylC(O)O, NHOC(O)NH, NC₁₋₄alkylOC(O), and an enzymatically cleavable peptide sequence. In some embodiments, the cleavable linker comprises one or more cleavable moieties selected from the group consisting of S—S, C(O)O, and OC(O).

In some embodiments, the cleavable linker comprises one or more groups selected from the group consisting of R⁵NC₁₋₂₀alkyleneNR⁶, R⁵NC₁₋₂₀alkenyleneNR⁵, C(O)C₁₋₂₀alkyleneC(O), C(O)C₁₋₂₀alkenyleneC(O), R⁵NC₁₋₂₀alkyleneC(O), R⁵NC₁₋₂₀alkenyleneC(O), C(O)C₁₋₂₀alkyleneNR⁶, C(O)C₁₋₂₀alkenyleneNR⁶, C(S)C₁₋₂₀alkyleneC(S), C(S)C₁₋₂₀alkenyleneC(S), C(S)C₁₋₂₀alkyleneC(O), C(S)C₁₋₂₀alkenyleneC(O), C(O)C₁₋₂₀alkyleneC(S), C(O)C₁₋₂₀alkenyleneC(S), SC₁₋₂₀alkyleneS, SC₁₋₂₀alkenyleneS, SC₁₋₂₀alkyleneNR⁶, SC₁₋₂₀alkenyleneNR⁶, R⁵NC₁₋₂₀alkyleneS, R⁵NC₁₋₂₀alkenyleneS, R⁵NC₁₋₂₀alkyleneO, R⁵NC₁₋₂₀alkenyleneO, OC₁₋₂₀alkyleneNR⁶, OC₁₋₂₀alkenyleneNR⁶, SC₁₋₂₀alkyleneO, SC₁₋₂₀alkenyleneO, OC₁₋₂₀alkyleneS, and OC₁₋₂₀alkenyleneS, C(O)C₁₋₂₀alkyleneO, C(O)C₁₋₂₀alkenyleneO, OC₁₋₂₀alkyleneC(O), OC₁₋₂₀alkenyleneC(O), C(O)C₁₋₂₀alkyleneS, C(O)C₁₋₂₀alkenyleneS, SC₁₋₂₀alkyleneC(O), SC₁₋₂₀alkenyleneC(O), R⁵NC₁₋₂₀alkyleneC(S), R⁵NC₁₋₂₀alkenyleneC(S), C(S)C₁₋₂₀alkyleneNR⁶, C(S)C₁₋₂₀alkenyleneNR⁶, C(S)C₁₋₂₀alkyleneO, C(S)C₁₋₂₀alkenyleneO, OC₁₋₂₀alkyleneC(S), OC₁₋₂₀alkenyleneC(S), SC₁₋₂₀alkyleneC(S), SC₁₋₂₀alkenyleneC(S), OC₁₋₂₀alkyleneO, OC₁₋₂₀alkenyleneCO, SC₁₋₂₀alkyleneS, and SC₁₋₂₀alkenyleneS, each of which is independently and optionally interrupted by one or more of S—S, C(O)O, OC(O), OC(O)O, OC(O)NH, NHC(O)O, SC(O), C(O)S, NC₁₋₄alkylC(O)O, NHOC(O)NH, NC₁₋₄alkylOC(O), C═NNH, C═NNH₂, C═NOH, C═NO, NH—NH, NH—NC₁₋₄alkyl, NC₁₋₄alkyl-NH, NC₁₋₄alkylNC₁₋₄alkyl, S, O, NH, N(C₁₋₆alkyl), C(O), C(O)NH, NHC(O), NHC(O)NH, NHC(S)NH, C(S)NH, NHC(S), NHC(NH), NHC(NC₁₋₄alkyl), C(NH)NH, C(NC₁₋₄alkyl)NH, NC₄₋₁₈cycloalkyl, C₄₋₁₀heterocycloalkyl, C₆₋₁₀aryl and C₅₋₁₀heteroaryl, and wherein each alkyl, alkylene or alkenylene is optionally substituted with one or more substituents selected from the group consisting of halo, COOH, C₁₋₆alkyl, OH, OC₁₋₆alkyl, SH, SC₁₋₆alkyl, NR⁷R⁷, C₁₋₄alkyleneOH, C₁₋₄alkyleneOC₁₋₄alkyl and C₁₋₄alkyleneNR⁷R⁸, wherein R⁵, R⁶, R⁷ and R⁸ are independently selected from the group consisting of H and C₁₋₄alkyl.

In some embodiments, the cleavable linker comprises one or more groups selected from the group consisting of R⁵NC₁₋₂₀alkyleneNR⁶, C(O)C₁₋₂₀alkyleneC(O), R⁵NC₁₋₂₀alkyleneC(O), C(O)C₁₋₂₀alkyleneNR⁶, R⁵NC₁₋₂₀alkyleneO, OC₁₋₂₀alkyleneNR⁶, C(O)C₁₋₂₀alkyleneO or OC₁₋₂₀alkyleneC(O), each of which is independently and optionally interrupted by one or more of S—S, C(O)O, OC(O), OC(O)O, OC(O)NH, NHC(O)O, SC(O), C(O)S, NC₁₋₄alkylC(O)O, NHOC(O)NH, NC₁₋₄alkylOC(O), S, O, NH, N(C₁₋₆alkyl), C(O), C(O)NH, NHC(O), NHC(O)NH, NHC(S)NH, C(S)NH, NHC(S), C₄₋₁₈cycloalkyl, C₄₋₁₀heterocycloalkyl, C₆₋₁₀aryl and C₅₋₁₀heteroaryl, and wherein each alkyl and alkylene is optionally substituted with one or more substituents selected from the group consisting of halo, COOH, C₁₋₆alkyl, OH, OC₁₋₆alkyl, SH, SC₁₋₆alkyl, NR⁷R⁷, C₁₋₄alkyleneOH, C₁₋₄alkyleneOC₁₋₄alkyl and C₁₋₄alkyleneNR⁷R⁸, wherein R⁵, R⁶, R⁷ and R⁸ are independently selected from the group consisting of H and C₁₋₄alkyl.

In some embodiments, the cleavable linker comprises one or more groups selected from the group consisting of R⁵NC₁₋₂₀alkyleneNR⁶, C(O)C₁₋₂₀alkyleneC(O), R⁵NC₁₋₂₀alkyleneC(O), C(O)C₁₋₂₀alkyleneNR⁶, R⁵NC₁₋₂₀alkyleneO, OC₁₋₂₀alkyleneNR⁶, C(O)C₁₋₂₀alkyleneO or OC₁₋₂₀alkyleneC(O), which optionally interrupted by one or more of S—S, C(O)O, OC(O), S, O, C(O)NH, NHC(O), C₄₋₁₈cycloalkyl, and C₄₋₁₀heterocycloalkyl, and each alkyl and alkylene is optionally substituted with one or more substituents selected from the group consisting of halo, COOH, NR⁷R⁷, and C₁₋₄alkyleneNR⁷R⁸, wherein R⁵, R⁶, R⁷ and R⁸ are independently selected from the group consisting of H and C₁₋₄alkyl.

In some embodiments, the cleavable linker comprises or is a structure of Formula (I-CL3):

—Y¹—W¹—Y²—W²—Y³—W³—Y⁴—W⁴—Y⁵—W⁵—Y⁶—, (I-CL3),
wherein each of W¹, W², W³, W⁴, or W⁵ is independently a bond, or unbranched or branched C₁₋₆alkylene;
wherein each of Y¹, Y², Y³, Y⁴, Y⁵, Y⁶ is independently a bond, O, S, S—S, C(O)O, OC(O), OC(O)O, OC(O)NR^{Y}, NR^{Y}C(O)O, NR^{Y}, C(O), C(O)NR^{Y}, NR^{Y}C(O), NR^{Y}C(O)NR^{Y}, C₅₋₇cycloalkyl, C₄₋₆heterocycloalkyl, or CH(RAA),
wherein each R^{Y} is independently H, C₁₋₆alkyl, C₁₋₆alkyl substituted with one or more independently selected halogen, C₁₋₆alkyleneOH, C₁₋₆alkyleneNR⁷R⁸, C₁₋₅alkyleneCOOH, or C₁₋₄alkyleneOC₁₋₄alkyl; and
wherein R^{AA} is a side chain of a naturally occurring amino acid;
provided that at least one of Y¹, Y², Y³, Y⁴, Y⁵, Y⁶ is not a bond.

In some embodiments, at least one of Y¹, Y², Y³, Y⁴, Y⁵, Y⁶ in Formula (I-CL3) is C(O)O, OC(O), OC(O)O, OC(O)NR^{Y}, or NR^{Y}C(O)O.

In some embodiments, the cleavable linker comprises one or more groups comprising a structure of Formula (I-CL1)

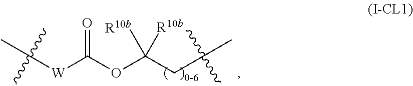

wherein W is selected from C(R^{10a})₂, N(R^{10a}) or O,
each R^{10a} is independently H, C₁₋₆alkyl, C₁₋₆alkyl substituted with one or more independently selected halogen, C₁₋₆alkyleneOH, C₁₋₆alkyleneNR⁷R⁸, C₁₋₅alkyleneCOOH, C₁₋₄alkyleneOC₁₋₄alkyl, or the side chain of a naturally occurring amino acid, or two R^{10a} groups, together with the carbon atom to which they are attached, form a C₃₋₁₀cycloalkyl or C₃₋₁₀heterocycloalkyl;
each R^{10b} is independently H, C₁₋₆alkyl, C₁₋₆alkyl substituted with one or more independently selected halogen, C₁₋₆alkyleneOH, C₁₋₆alkyleneNR⁷R⁸, C₁₋₅alkyleneCOOH, C₁₋₄alkyleneOC₁₋₄alkyl, or two R^{10b} groups, together with the carbon atom to which they are attached, form a C₃₋₁₀cycloalkyl or C₃₋₁₀heterocycloalkyl; and
R⁷ and R⁸ are each independently H, or C₁₋₄alkyl.

In some embodiments, the cleavable linker comprises at least one group selected

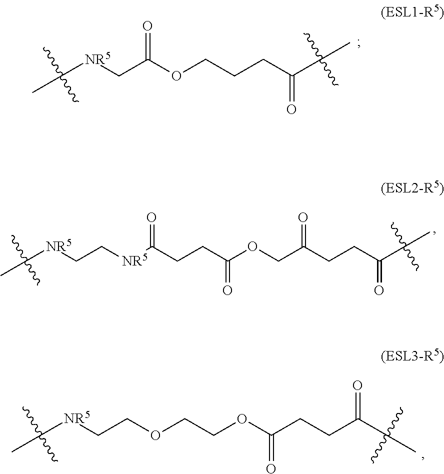

wherein each R⁵ is selected from the group consisting of H and C₁₋₄alkyl.

In some embodiments, the cleavable linker comprises one or more groups selected from the group consisting of

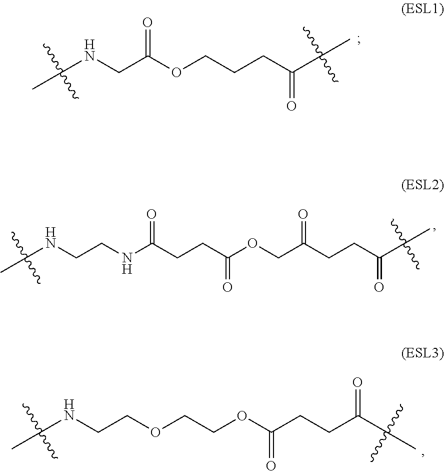 [SMILES: *NCC(=O)OCCCC(*)=O ; *NCCNC(=O)CCC(=O)OCC(=O)CCC(*)=O ; *NCCOCCOC(=O)CCC(*)=O]

In some embodiments, the cleavable linker comprises one or more groups having a structure of Formula (I-CL2)

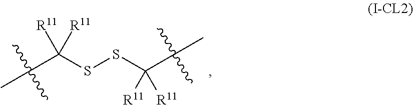

and wherein each R¹ is independently selected from H or C₁₋₄alkyl. In some embodiments, the cleavable linker comprises one or more groups of

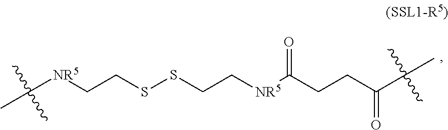

wherein each R⁵ is selected from the group consisting of H and C₁₋₄alkyl. In some embodiments, the cleavable linker comprises one or more groups selected from the group consisting of

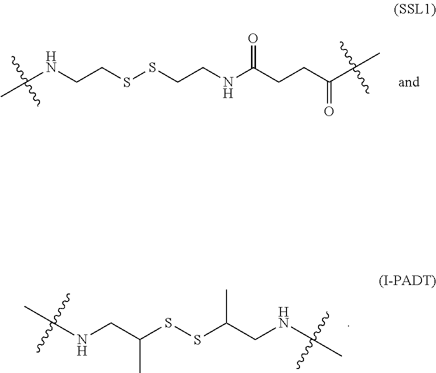 [SMILES: *NCCSSCCNC(=O)CCC(*)=O ; *NCC(C)SSC(C)CN*]

In some embodiments, cleavable linker comprises an enzymatically cleavable peptide sequence. In some embodiments, the enzymatically cleavable peptide sequence is selected from the group consisting of Met-Val-Lys, Ala-Val, Ala-Pro, Gly-Pro, Ser-Ser, Ser-Gly, Gly-Ala and Ser-Lys.

In some embodiments, L^{Z(i)} of the compound as described herein comprises a thiol-reactive group or an amine reactive group.

In some embodiments, L^{Z(i)} comprises a thiol-reactive group. In some embodiments, the thiol-reactive group is selected from the group consisting of a thiol-reactive double bond, a thiol-reactive triple bond, and a leaving group containing moiety. In some embodiments, the thiol-reactive group is selected from the group consisting of

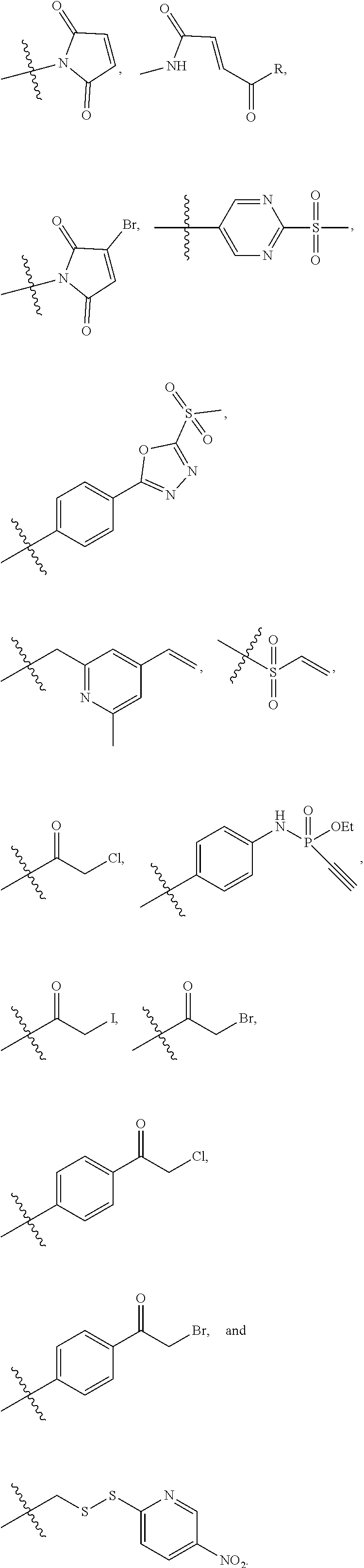

In some embodiments, L^{Z(i)} comprises an amine-reactive group. In some embodiments, the amine-reactive group is selected from the group consisting of

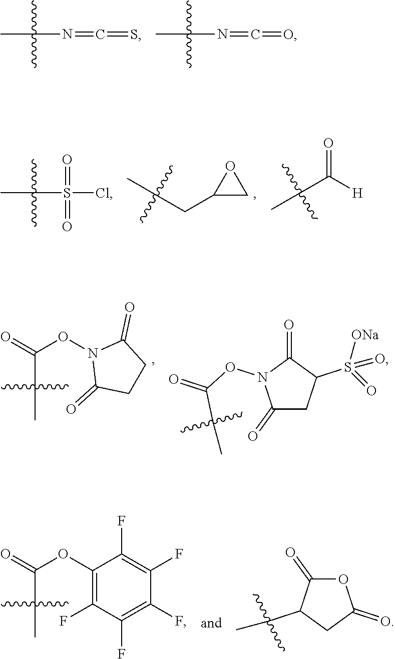

In some embodiments, L^{Z(i)} further comprises one or more groups selected from the group consisting of R¹NC₁₋₂₀alkyleneNR², R¹NC₁₋₂₀alkenyleneNR² C(O)C₁₋₂₀alkyleneC(O), C(O)C₁₋₂₀alkenyleneC(O), R¹NC₁₋₂₀alkyleneC(O), R¹NC₁₋₂₀alkenyleneC(O), C(O)C₁₋₂O alkyleneNHR², C(O)C₁₋₂₀alkenyleneNR², C(S)C₁₋₂₀alkyleneC(S), C(S)C₁₋₂₀alkenyleneC(S), C(S)C₁₋₂₀alkyleneC(O), C(S)C₁₋₂₀alkenyleneC(O), C(O)C₁₋₂₀alkyleneC(S), C(O)C₁₋₂₀alkenyleneC(S), C(O)C₁₋₂₀alkyleneO, C(O)C₁₋₂₀alkenyleneO, R¹NC₁₋₂₀alkenyleneC(S), C(S)C₁₋₂₀alkyleneNR², and C(S)C₁₋₂₀alkenyleneNR², each of which is independently and optionally interrupted by one or more of S, O, NH, N(C₁₋₆alkyl), C(O), C(O)NH, NHC(O), C(S)NH, NHC(S), NHC(O)NH, NHC(S)NH, NHC(NH), NHC(NC₁₋₄alkyl), C(NH)NH, C(NC₁₋₄alkyl)NH, NC₄₋₁₀cycloalkyl, C₄₋₁₀heterocycloalkyl, C₆₋₁₀aryl and C₅₋₁₀heteroaryl, and wherein each alkyl, alkylene and alkenylene is optionally substituted with one or more substituents selected from the group consisting of halo, COOH, C₁₋₆alkyl, OH, OC₁₋₆alkyl, SH, SC₁₋₆alkyl, NR³R⁴, C₁₋₄alkyleneOH, C₁₋₄alkyleneOC₁₋₄alkyl and C₁₋₄alkyleneNR³R⁴, wherein each R¹, R², R³ and R⁴ is independently selected from the group consisting of H and C₁₋₄alkyl.

In some embodiments, L^{Z(i)} comprises one or more amino acid residues. In some embodiments, the amino acid residue of L^{Z(i)} is derived from a naturally occurring amino acid selected from the group consisting of alanine (Ala, A), arginine (Arg, R), asparagine (Asn, N), aspartic acid (Asp, D), glutamine (Gln, Q), glutamic acid (Glu, E), γGlu, glutamine (Gln), glycine (Gly, G), histidine (His, H), isoleucine (Ile, I), leucine (Leu, L), Lysine (Lys, K), εLys, methionine (Met, M), phenylalanine (Phe, F), proline (Pro, P), serine (Ser, S), threonine (Thr, T), tryptophan (Trp, W), tyrosine (Tyr, Y), valine (Val, V), pyrrolysine (Pyl, O), pyrroline-carboxy-lysine (PCL) gamma-carboxyglutamic acid (Gla), and a D enantiomer thereof.

In some embodiments, the amino acid residue of L^{Z(i)} is derived from a unnaturally occurring amino acid selected from the group consisting of NR⁵C₅alkyleneC(O) (Ahx), L-Cysteic acid (Cya), norleucine (Nle), norvaline (Nva), 2-aminooctanoic acid (Aoc), 2-naphthylalanine (2-Nal), 3-(trifluoromethyl)phenylalanine (TFP), homophenylalanine (hPhe), cyclohexylalanine (Cha), 1-naphthylalanine (1-Nal), 4-benzoyl-L-phenylalanine (Bpa), 2-methoxy-4-vinylphenylalanine (MvF), 4-fluorophenylalanine (4-F-Phe), 4-phenyl-2,3-dihydroxy-6-nitrophenylalanine (pNIPA), 2-(2-naphthyl)alanine (2-Nal-ala), 4-(4-propoxyphenyl)alanine (Ppa), 4-carboxyphenylalanine (4-CPA), 4-butylphenylalanine (Bua), 2-nitrophenylalanine (2-Npa), 4-azidophenylalanine (4-AzF), 2-(4-nitrophenyl)ethylalanine (2-Npe), 3-iodo-L-tyrosine (Ity), 5,5,5-trifluoroleucine (TFL), and a D enantiomer thereof.

In some embodiments, L^{Z(i)} of the compound as described herein further comprises —N(R^{N})(C₂alkyleneO)ₓ₁X^{b}C(O), wherein R^{N} is H or C₁-C₆ alkyl, x1 is an integer from 1 to 10, and X^{b} is C₁-C₃ alkylene. In some embodiments, the —N(R^{N})(C₂alkyleneX^{a})ₓ₁X^{b}C(O)— is selected from the group consisting of —N(R^{N})(C₂alkyleneO)₂C₁alkyleneC(O), —N(R^{N})(C₂alkyleneO)C₂alkyleneC(O), —N(R^{N})(C₂alkyleneO)₃C₂alkyleneC(O), and —N(R^{N})(C₂alkyleneO)₆C₂alkyleneC(O), wherein R^{N} is H or C₁-C₆ alkyl. In some embodiments, R^{N} is H. In some embodiments, R^{N} is methyl, ethyl, or propyl. one of more moieties of —(OCH₂CH₂)ₙ—, wherein n is an integer of 1-28.

In some embodiments, L^{Z(i)} comprises one or more moieties each independently selected from the group consisting of —(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), and —(C₂alkyleneO)₆C₂alkyleneC(O) (PEG6), —(C₂alkyleneO)₆C₂alkyleneC(O) (PEG8), and —(C₂alkyleneO)₆C₂alkyleneC(O) (PEG12).

In some embodiments, the compound as described herein comprises a branching group T which is at least trivalent. In some embodiments, T of the compound of the present application is at least trivalent. In some embodiments, T of the compound of the present application is at least tetravalent. In some embodiments, T of the compound comprises one or more amino acid residues each independently derived from the group consisting of Lys, ornithine (Orn), homo-lysine, 2,3-diaminopropionic acid (Dap), 2,4-diaminobutyric acid (Dab), cysteine, homo-cysteine, glutamine, glutamic acid, asparagine, aspartic acid, 3,5-bis(aminomethyl)benzoic acid (Bab), 4-aminomethylphenylalaline (Amp), 4R-4-aminoproline (Apr), 4-(2-aminoethoxy)phenylalanine, 4-aminopiperidine-4-carboxylic acid (Apc), 2-((1,3-diaminopropan-2-yl)oxy)acetic acid (Dpa), and a D enantiomer thereof.

In some embodiments, T further comprises a first terminal functionality, a second terminal functionality and a third terminal functionality, which are the same or different and bind to L^{Z(i)}, L^{A} (or alternatively A), and L^{E} (or alternatively E), respectively to independently form an amide group, an amine group, a urea group, a thioether group, a thiourea group or a thioamide group.

In some embodiments, provided at least one of L^{A} and L^{E} is a cleavable linker, T binds to L^{A} (or alternatively A), and/or L^{E} (or alternatively E) to further form an ester group, a thioester group, a carbonate group, a carbamate group, a disulfide bond, a hydrazone group, or an oxime group. In some embodiments, provided L^{A} is a cleavable linker, T binds to L^{A} to further form an ester group, a thioester group, a carbonate group, a carbamate group, a disulfide bond, a hydrazone group, or an oxime group. In some embodiments, provided L^{E} is a cleavable linker, T binds to L^{E} to further form an ester group, a thioester group, a carbonate group, a carbamate group, a disulfide bond, a hydrazone group, or an oxime group.

In some embodiments, T of the compound is a branching group which is trivalent, tetravalent or pentavalent. In some embodiments, T is a branching group which is trivalent. Therefore, in some embodiments, T is a trivalent branching group. In some embodiments, T is a branching group which is tetravalent. Therefore, in some embodiments, T is a tetravalent branching group.

In some embodiments, T is at least tetravalent further comprising a fourth terminal functionality to bind to a detection moiety (D) via a linker LD. In some embodiments, the fourth terminal functionality binds to L^{D} (or alternatively D) to form an amide group, a urea group, a thiourea group or a thioamide group.

Accordingly, in some embodiments, T is a tetravalent branching group and the compound of Formula I further includes a detection moiety (D) and D is conjugated to T via a linker LD, and the conjugate of Formula I is a conjugate of Formula I(a-X):

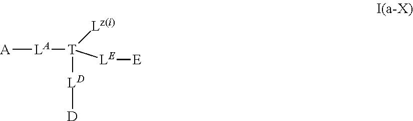

wherein:
T is a tetravalent branching group,
A, E, L^{E}, L^{A}, L^{Z(i)} are as defined in Formula (I-X),
D is a detection moiety, wherein D is conjugated to T via a linker L^{D}.

In some embodiments, D comprises an aromatic ring. In some embodiments, the aromatic ring is a benzene ring. In some embodiments, D comprises a tyrosine (Tyr) residue, a tryptophan (Trp) residue or a phenylalanine (Phe) residue. In some embodiments, D is detectable by a UV detector. In some embodiments, D is detectable by HPLC.

In some embodiments, D is

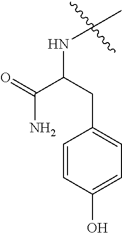 [SMILES: *NC(Cc1ccc(O)cc1)C(N)=O]

or the R- or S- stereoisomer thereof.

In some embodiments, L^{D} is a cleavable liner, direct bond or a non-cleavable linker as described herein. In some embodiments, L^{D} is selected from the group consisting of amine bonds, ether bonds, thioether bonds, amide bonds, thioamide, urea and thiourea bonds.

In some embodiments, T is at least tetravalent and comprises two or more amino acid residues derived from Lys, ornithine (Orn), homo-lysine, 2,3-diaminopropionic acid (Dap), 2,4-diaminobutyric acid (Dab), cysteine, homo-cysteine, glutamine, glutamic acid, asparagine, aspartic acid, 3,5-bis(aminomethyl)benzoic acid (Bab), 4-aminomethylphenylalaline (Amp), 4R-4-aminoproline (Apr), 4-(2-aminoethoxy)phenylalanine, 4-aminopiperidine-4-carboxylic acid (Apc), 2-((1,3-diaminopropan-2-yl)oxy)acetic acid (Dpa), or a D enantiomer thereof. In some embodiments, T is tetravalent and comprises two amino acid residues derived from Lys or a D enantiomer thereof.

In some embodiments, the compound of the present application comprises any of the structures as listed in Table 2.

TABLE 2
Exemplary structures of the compound of the present application

| No. | Seq/Structure |
|---|---|
| L001 | 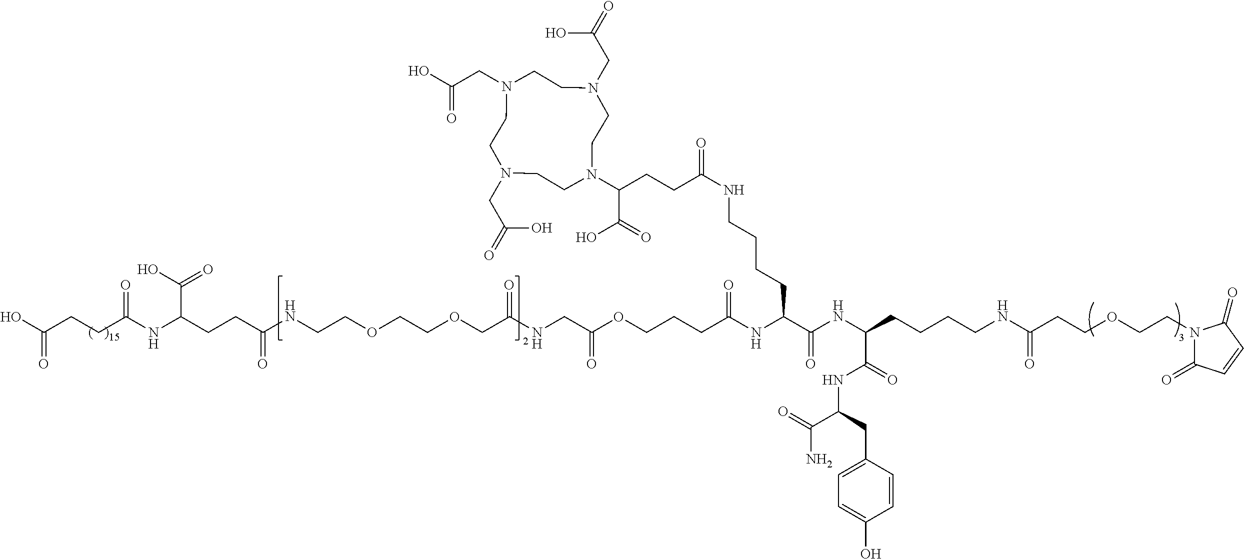 |
| | HO-C18-gGlu-OEG-OEG-ESL1-Lys(DOTAGA)-Lys(PEG3-MAL)-Tyr-NH2 |
| L002 | 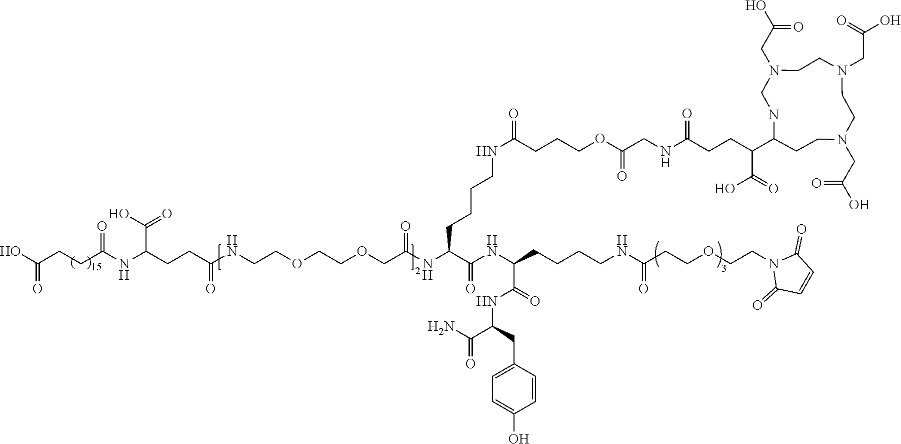 |
| | HO-C18-gGlu-OEG-OEG-Lys(ESL1-DOTAGA)-Lys(PEG3-MAL)-Tyr-NH2 |
| L003 | 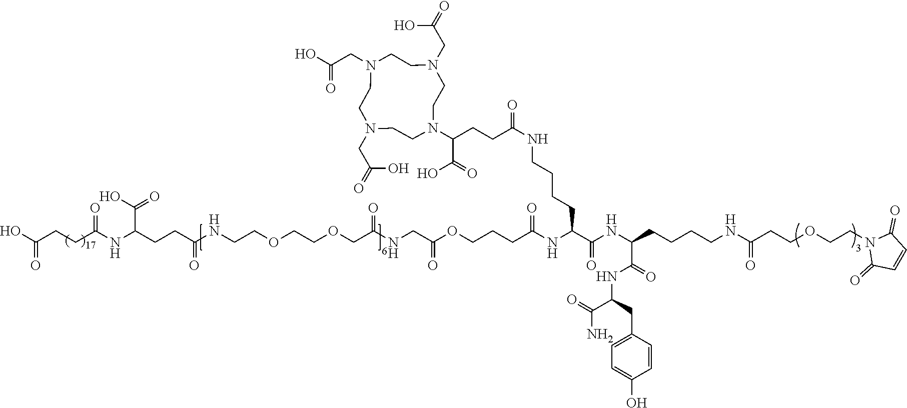 |
| | HO-C20-gGlu-OEG-OEG-OEG-OEG-OEG-OEG-ESL1-Lys(DOTAGA)-Lys(PEG3-MAL)-Tyr-NH2 |
| L004 | 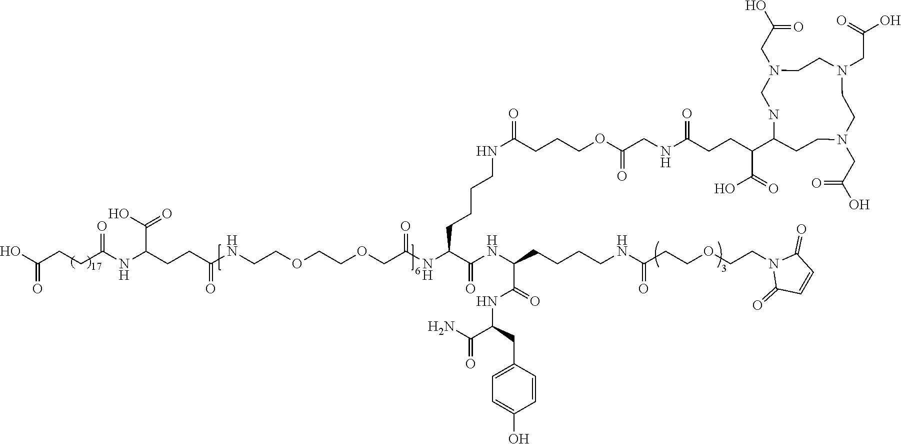 |
| | HO-C20-gGlu-OEG-OEG-OEG-OEG-OEG-OEG-Lys(ESL1-DOTAGA)-Lys(PEG3-MAL)-Tyr-NH2 |
| L005 | 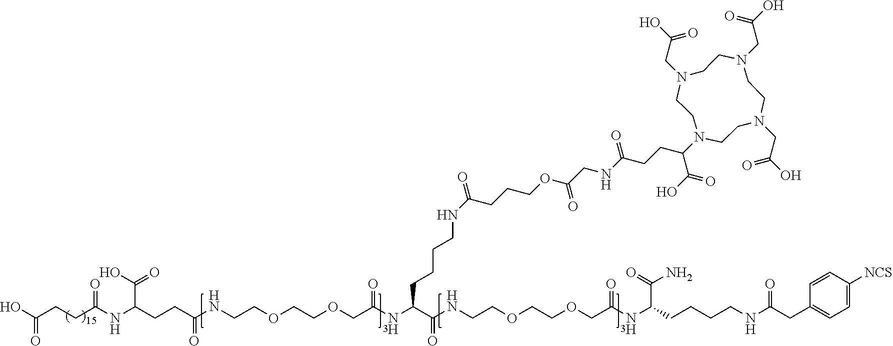 |
| | HO-C18-gGlu-OEG-OEG-OEG-Lys(ESL1-DOTAGA)-OEG-OEG-OEG-Lys[CO-CH2-C6H4-(pNCS)]-NH2 |
| L006 | 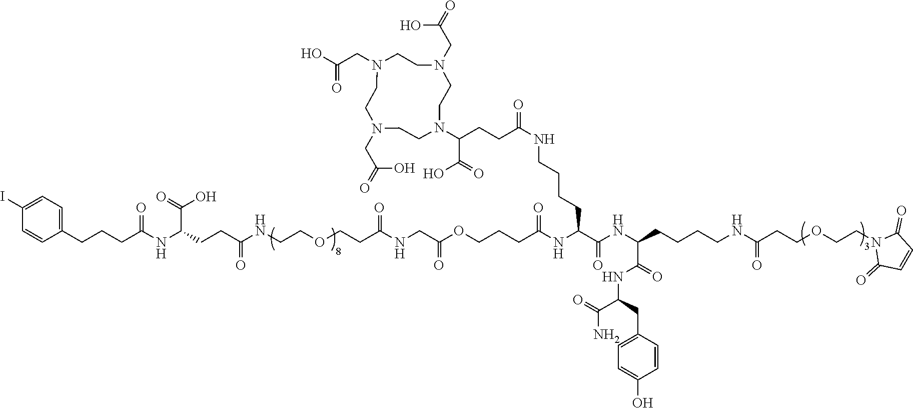 |
| | IPhBu-gGlu-PEG8-ESL1-Lys(DOTAGA)-Lys(PEG3-MAL)-Tyr-NH2 |
| L007 | 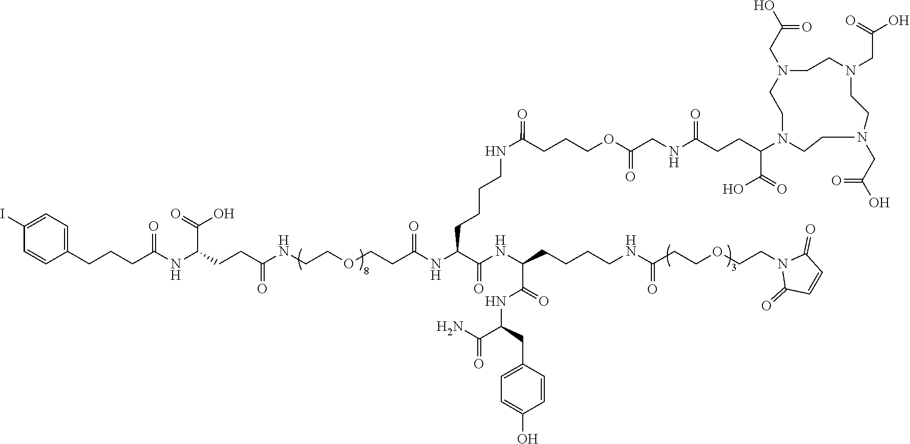 |
| | IPhBu-gGlu-PEG8-Lys(ESL1-DOTAGA)-Lys(PEG3-MAL)-Tyr-NH2 |
| L008 | 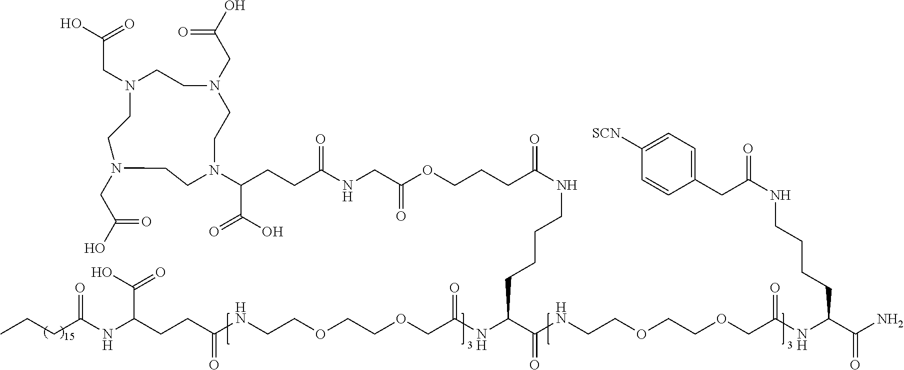 |
| | C18-gGlu-OEG-OEG-OEG-Lys(ESL1-DOTAGA)-OEG-OEG-OEG-Lys[CO-CH2-C6H4-(pNCS)]-NH2 |
| L009 | 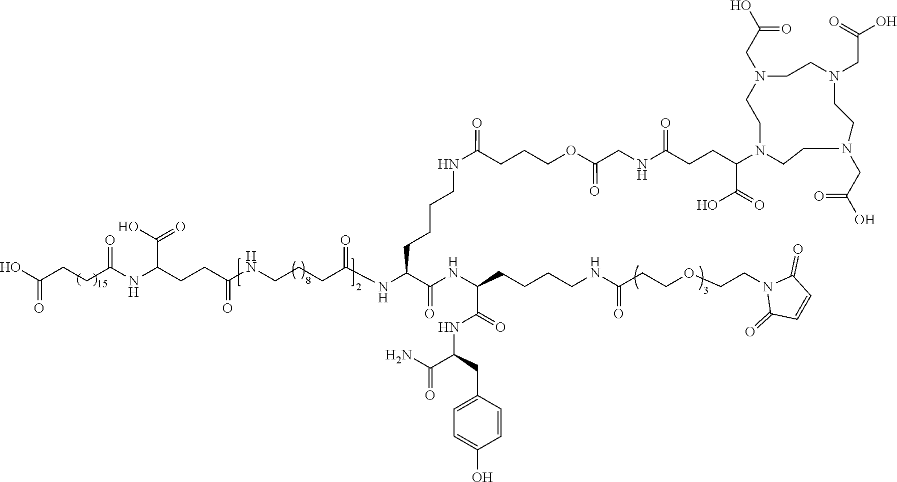 |
| | HO-C18-gGlu-Aun-Aun-Lys(ESL1-DOTAGA)-Lys(PEG3-MAL)-Tyr-NH2 |
| L010 | 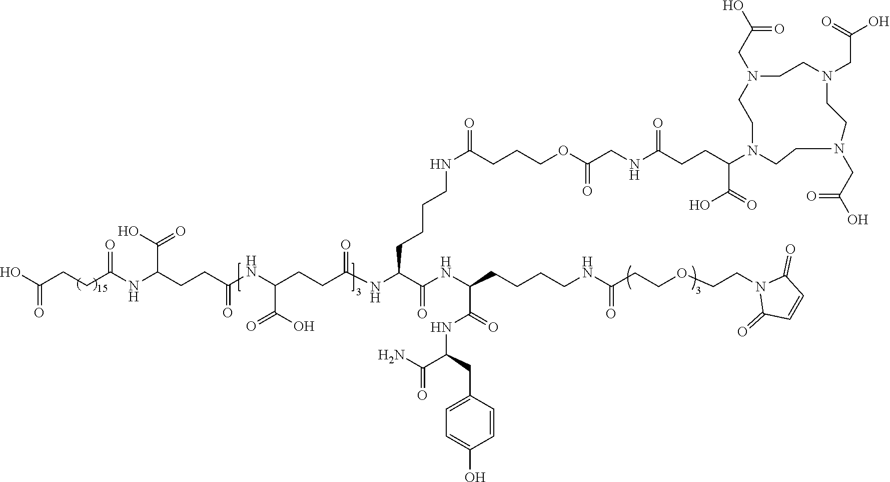 |
| | HO-C18-gGlu-gGlu-gGlu-gGlu-Lys(ESL1-DOTAGA)-Lys(PEG3-MAL)-Tyr-NH2 |
| L011 | 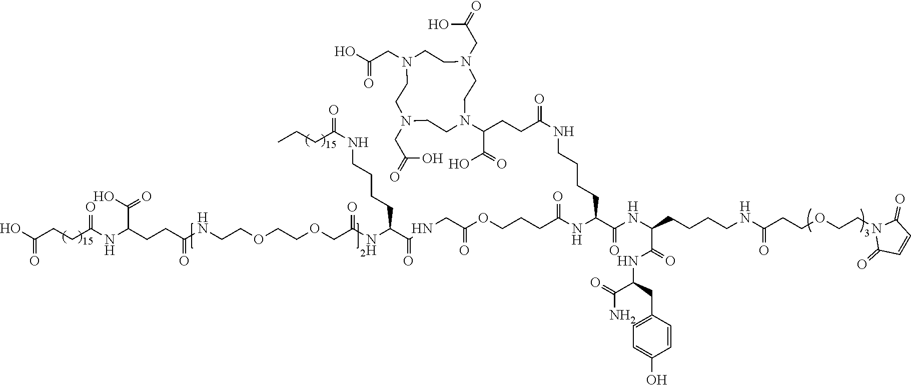 |
| | HO-C18-gGlu-OEG-OEG-Lys(C18)-ESL1-Lys(DOTAGA)-Lys(PEG3-MAL)-Tyr-NH2 |
| L012 | 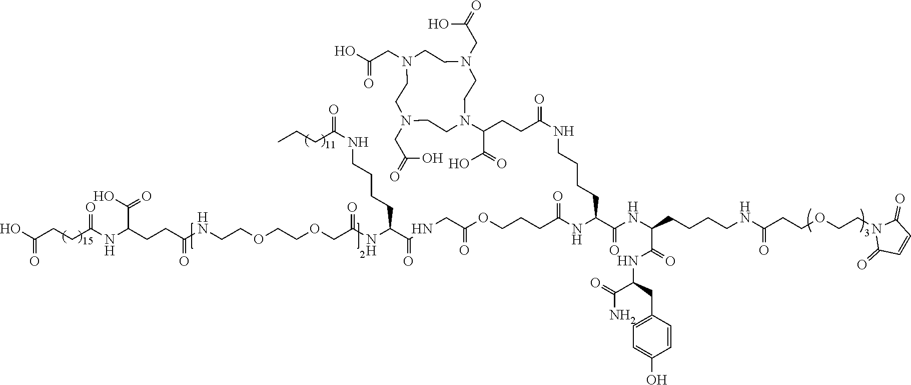 |
| | HO-C18-gGlu-OEG-OEG-Lys(C14)-ESL1-Lys(DOTAGA)-Lys(PEG3-MAL)-Tyr-NH2 |
| L013 | 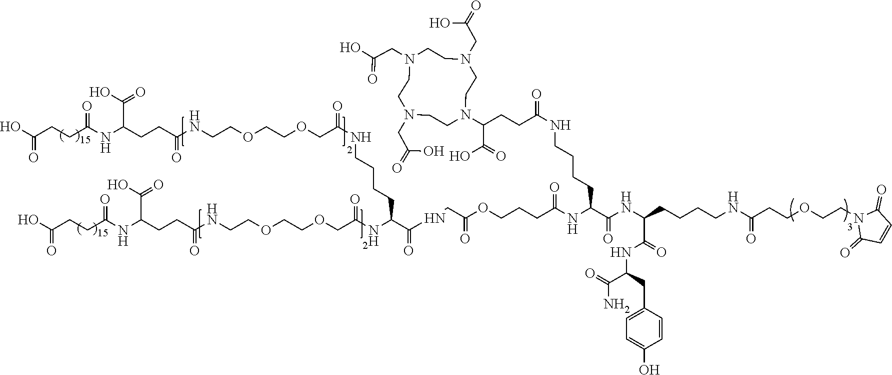 |
| | [HO-C18-gGlu-OEG-OEG]2-Lys-ESL1-Lys(DOTAGA)-Lys(PEG3-MAL)-Tyr-NH2 |
| L014 | 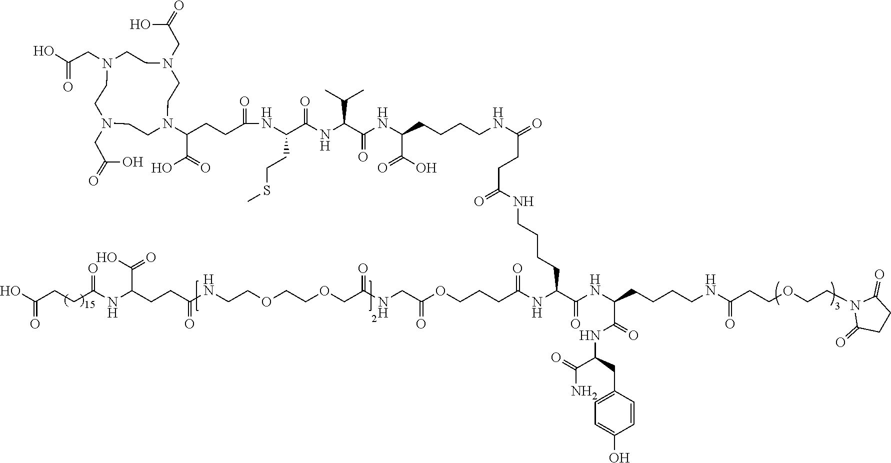 |
| | HO-C18-gGlu-OEG-OEG-ESL1-Lys(SA-eLys-Val-Met-DOTAGA)-Lys(PEG3-MAL)-Tyr-NH2 |
| L015 | 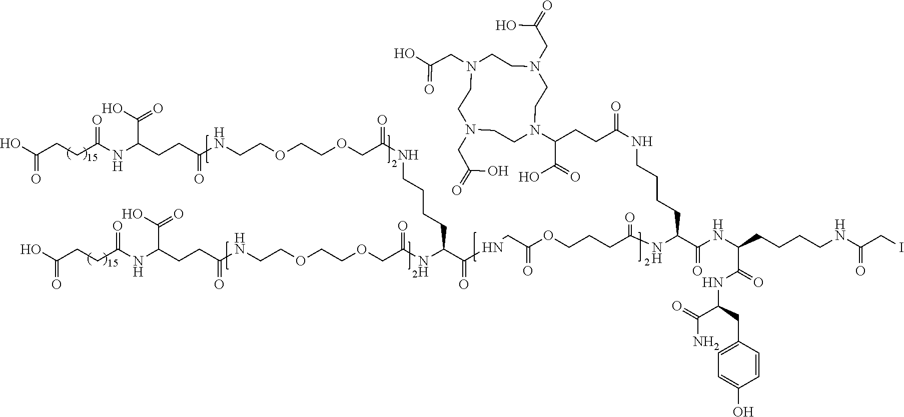 |
| | [HO-C18-gGlu-OEG-OEG]2-Lys-ESL1-ESL1-Lys(DOTAGA)-Lys(COCH2I)-Tyr-NH2 |
| L016 | 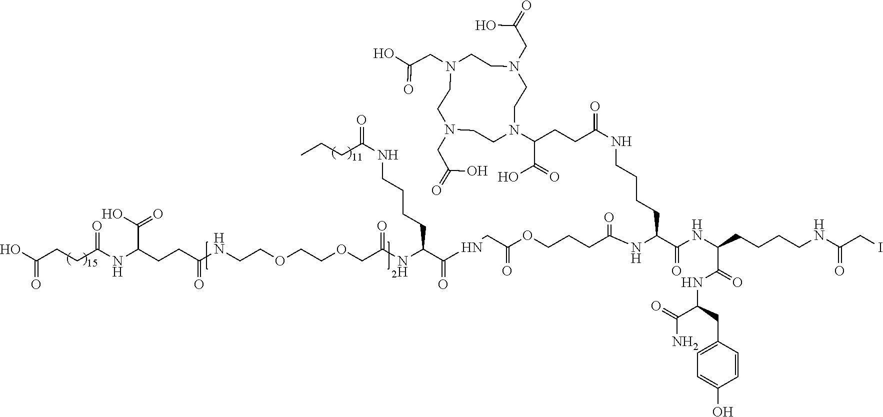 |
| | HO-C18-gGlu-OEG-OEG-Lys(C14)-ESL1-Lys(DOTAGA)-Lys(COCH2I)-Tyr-NH2 |
| L017 | 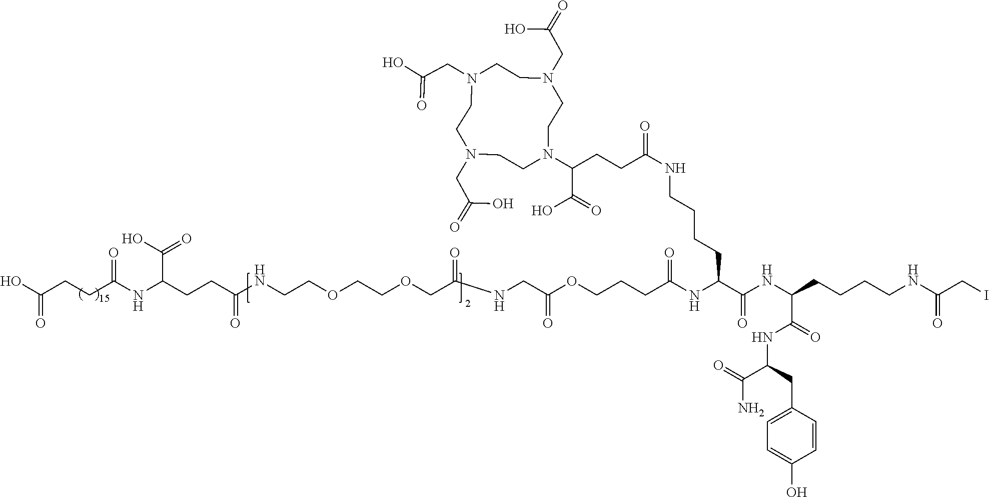 |
| | HO-C18-gGlu-OEG-OEG-Lys(DOTAGA)-Lys(COCH2I)-Tyr-NH2 |
| L018 | 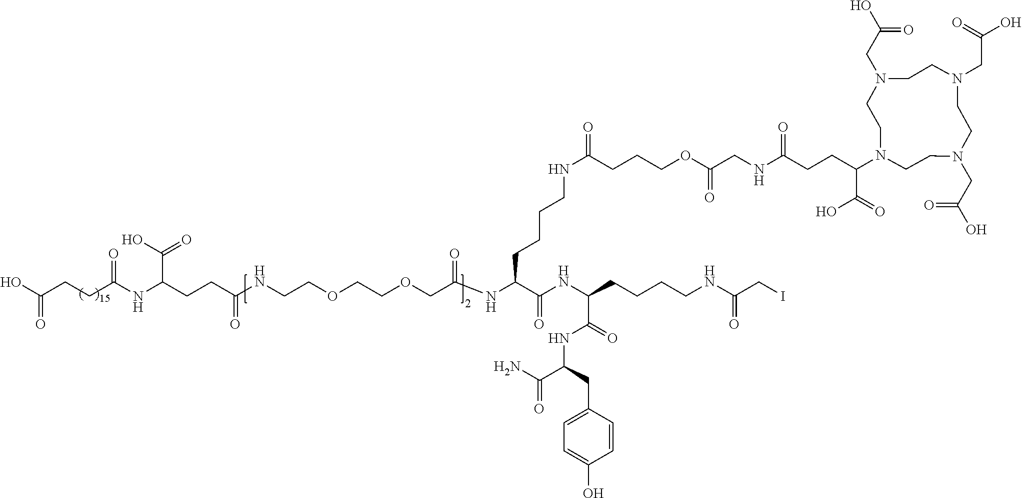 |
| | HO-C18-gGlu-OEG-OEG-Lys(ESL1-DOTAGA)-Lys(COCH2I)-Tyr-NH2 |
| L019 | 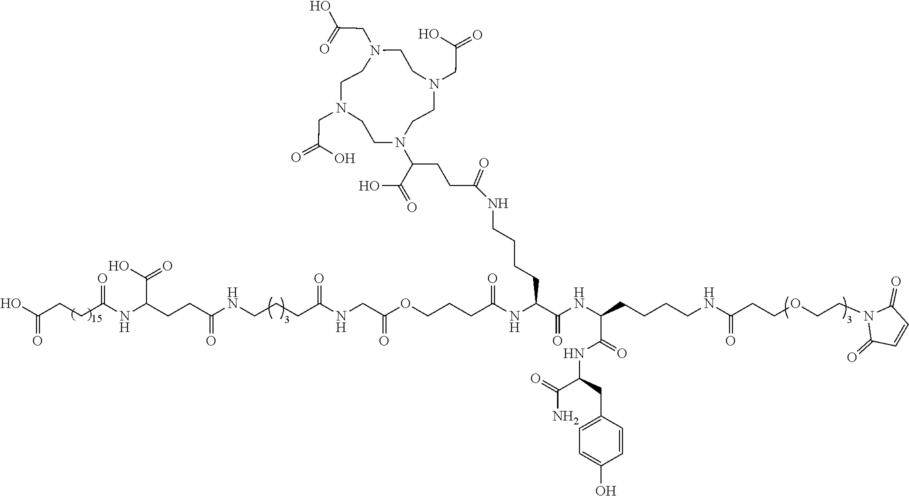 |
| | HO-C18-gGlu-Ahx-ESL1-Lys(DOTAGA)-Lys(PEG3-MAL)-Tyr-NH2 |
| L020 | 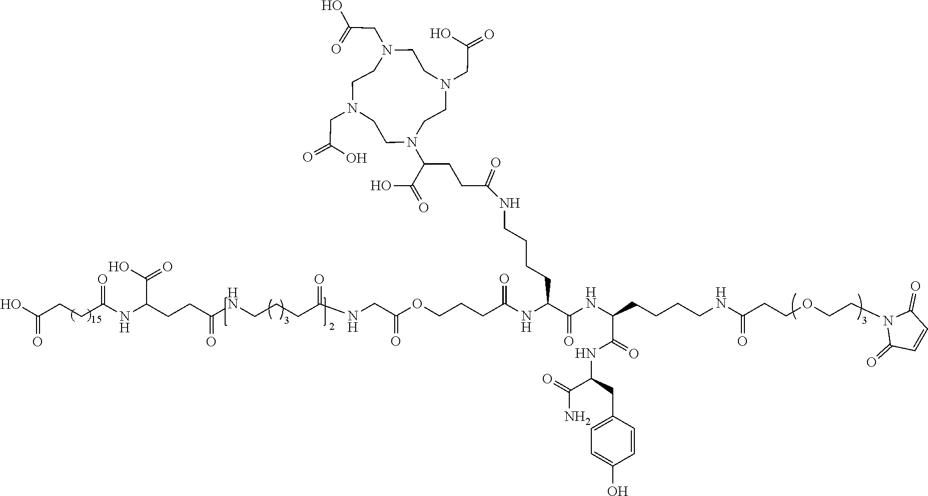 |
| | HO-C18-gGlu-Ahx-Ahx-ESL1-Lys(DOTAGA)-Lys(PEG3-MAL)-Tyr-NH2 |
| L021 | 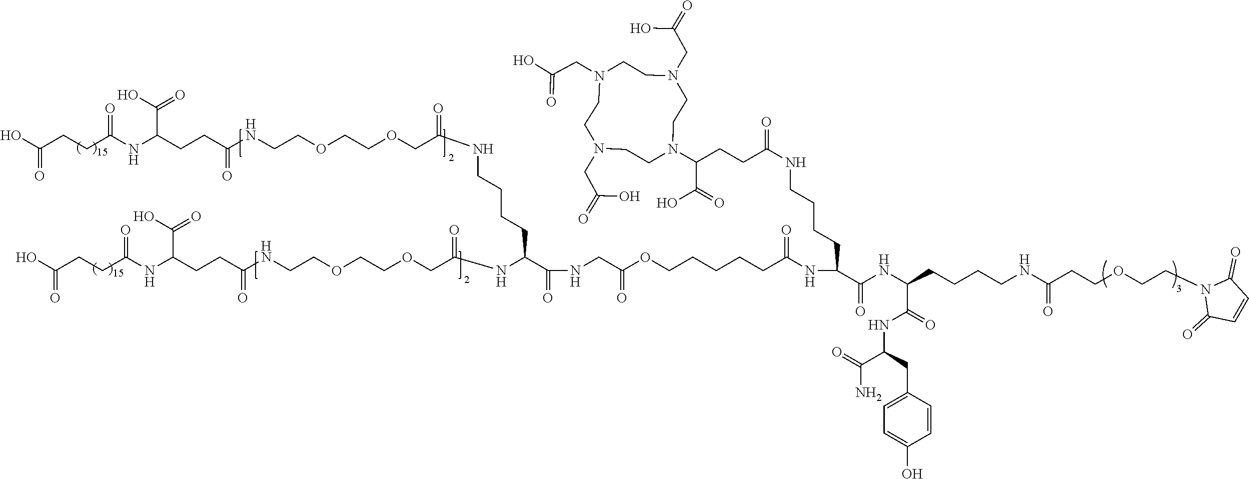 |
| | [HO-C18-gGlu-OEG-OEG]2-Lys-Gly-6hHA-Lys(DOTAGA)-Lys(PEG3-MAL)-Tyr-NH2 |
| L022 | 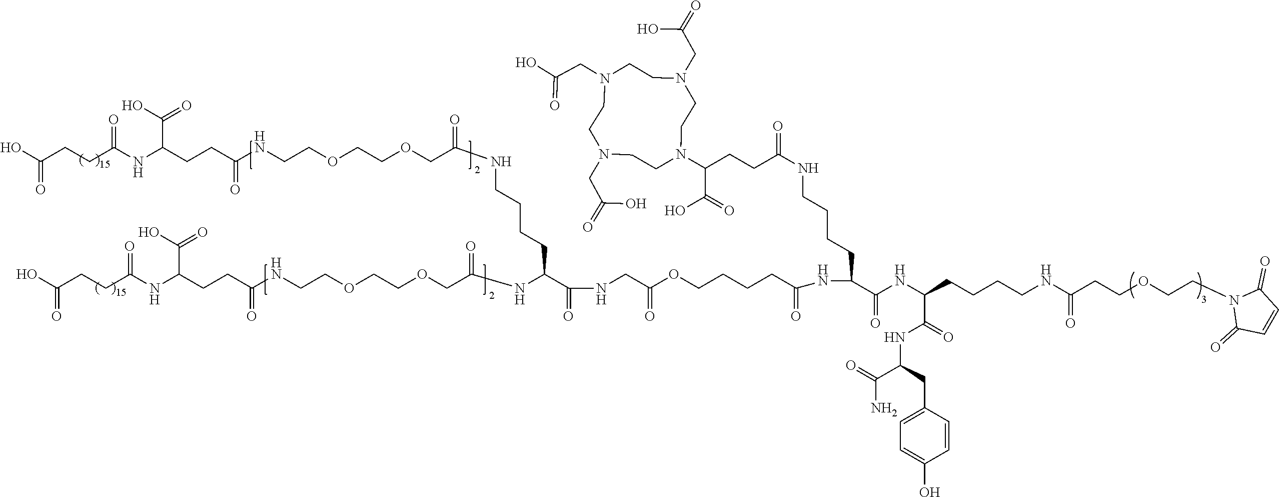 |
| | [HO-C18-gGlu-OEG-OEG]2-Lys-Gly-5hPA-Lys(DOTAGA)-Lys(PEG3-MAL)-Tyr-NH2 |
| L023 | 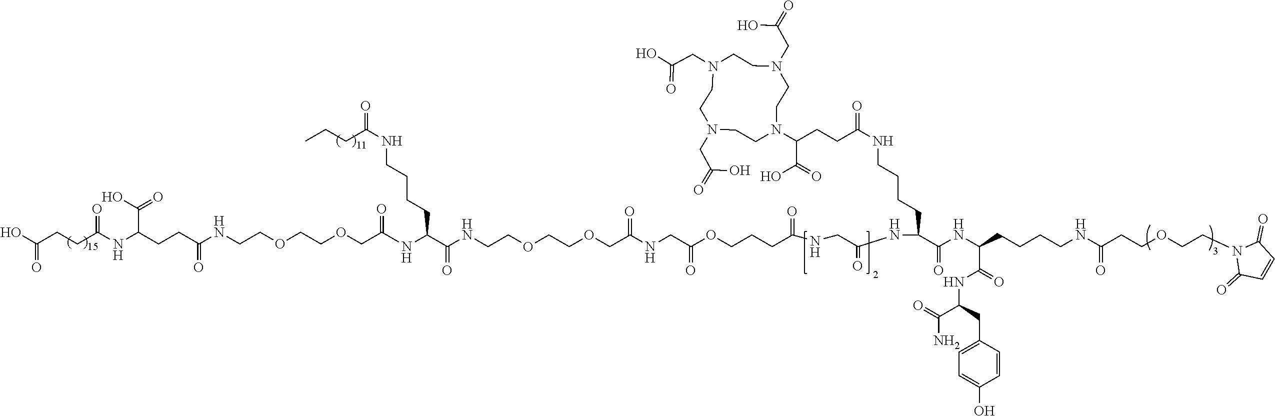 |
| | HO-C18-gGlu-OEG-Lys(C14)-OEG-ESL1-Gly-Gly-Lys(DOTAGA)-Lys(PEG3-MAL)-Tyr-NH2 |
| L024 | 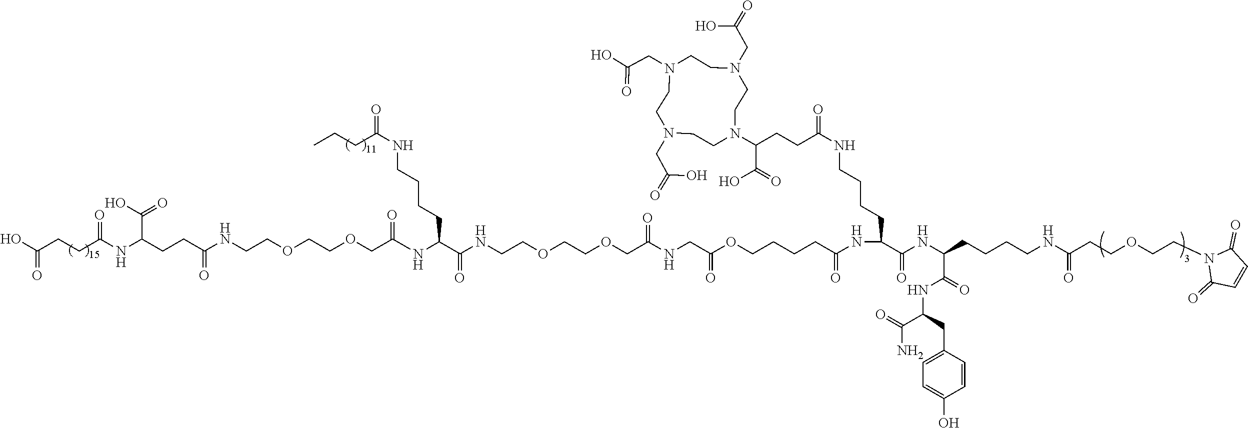 |
| | HO-C18-gGlu-OEG-Lys(C14)-OEG-Gly-5hPA-Lys(DOTAGA)-Lys(PEG3-MAL)-Tyr-NH2 |
| L025 | 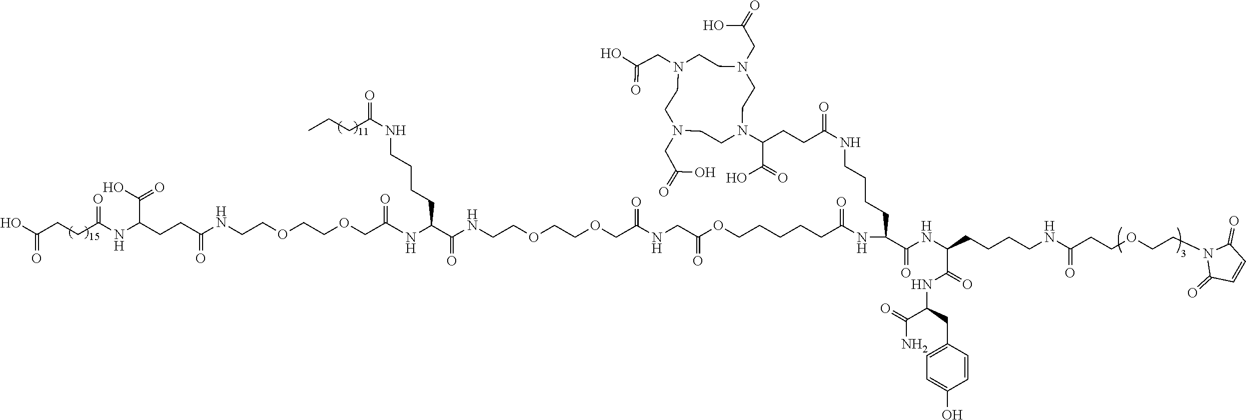 |
| | HO-C18-gGlu-OEG-Lys(C14)-OEG-Gly-6hHA-Lys(DOTAGA)-Lys(PEG3-MAL)-Tyr-NH2 |
| L026 | 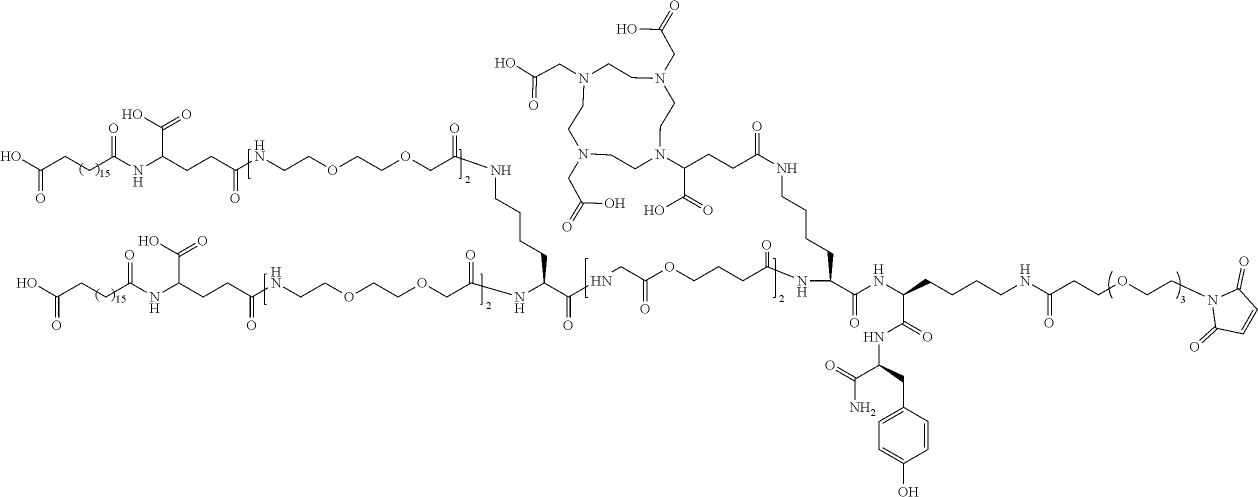 |
| | [HO-C18-gGlu-OEG-OEG]2-Lys-ESL1-ESL1-Lys(DOTAGA)-Lys(PEG3-MAL)-Tyr-NH2 |
| L027 | 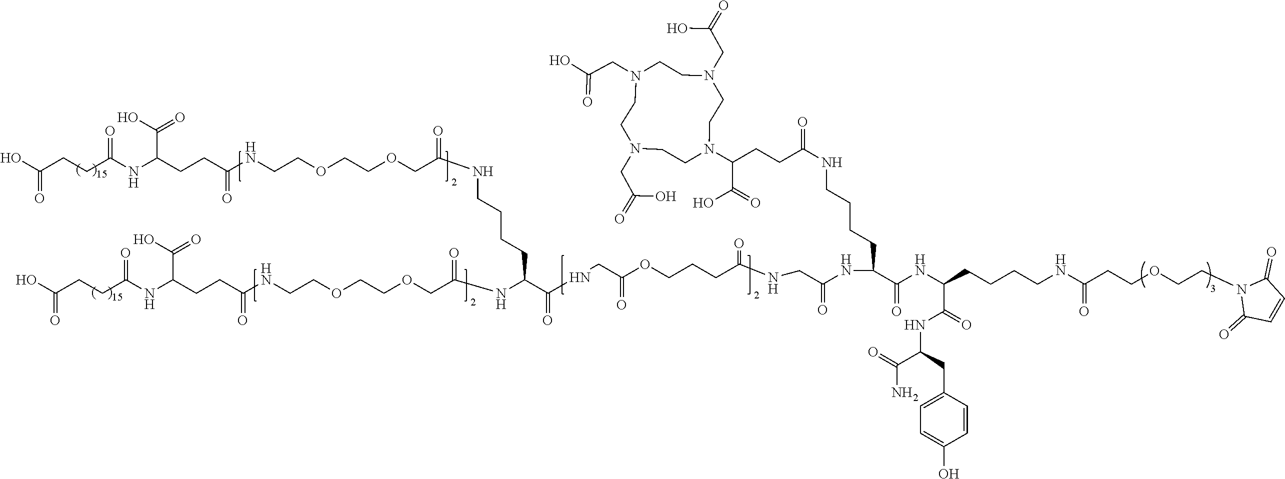 |
| | [HO-C18-gGlu-OEG-OEG]2-Lys-Gly-Lys(DOTAGA)-Lys(PEG3-MAL)-Tyr-NH2 |
| L028 | 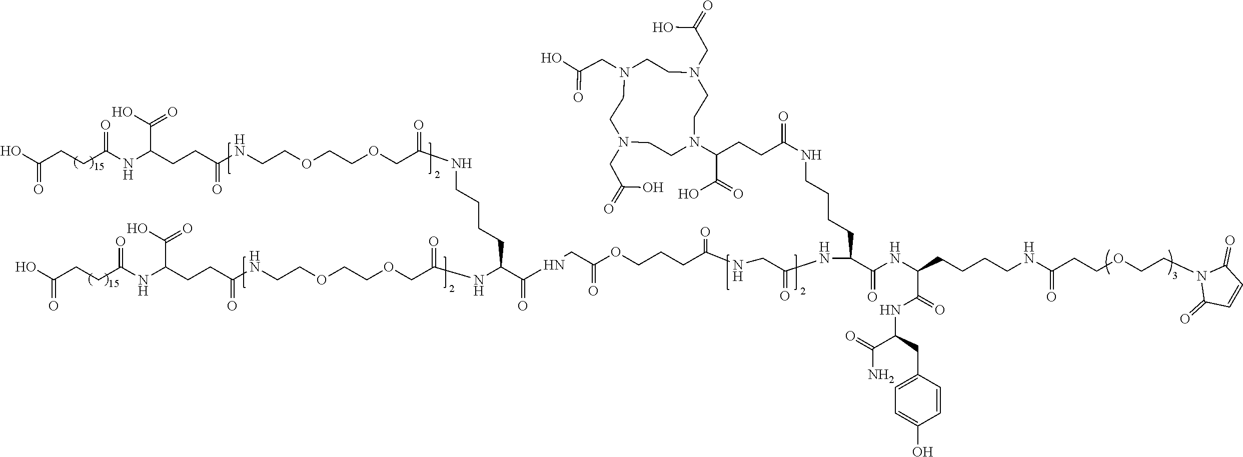 |
| | [HO-C18-gGlu-OEG-OEG]2-Lys-ESL1-Gly-Gly-Lys(DOTAGA)-Lys(PEG3-MAL)-Tyr-NH2 |
| L029 | 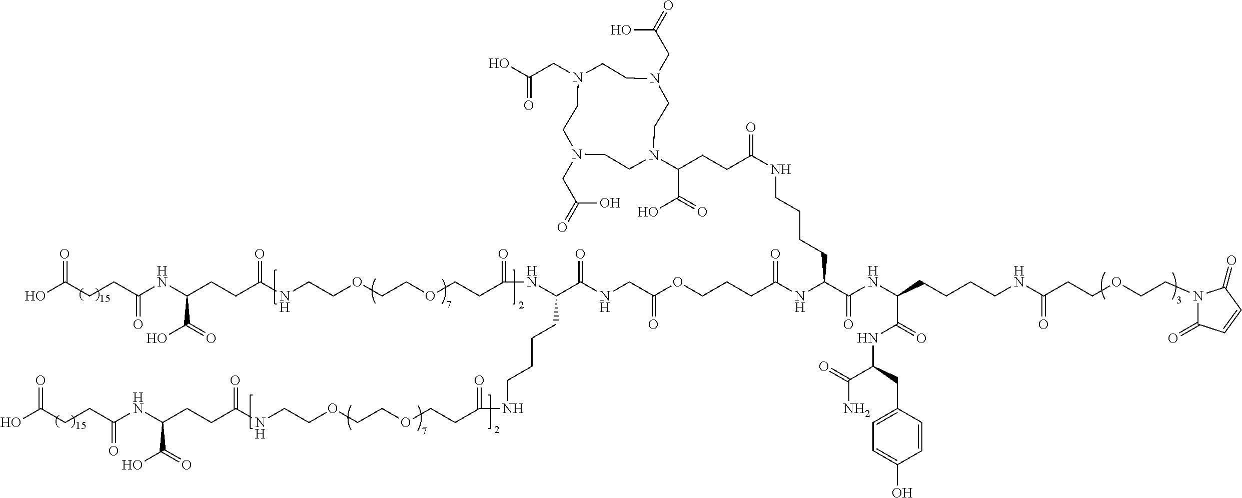 |
| | [HO-C18-gGlu-PEG8-PEG8]2-Lys-ESL1-Lys(DOTAGA)-Lys(PEG3-MAL)-Tyr-NH2 |
| L030 | 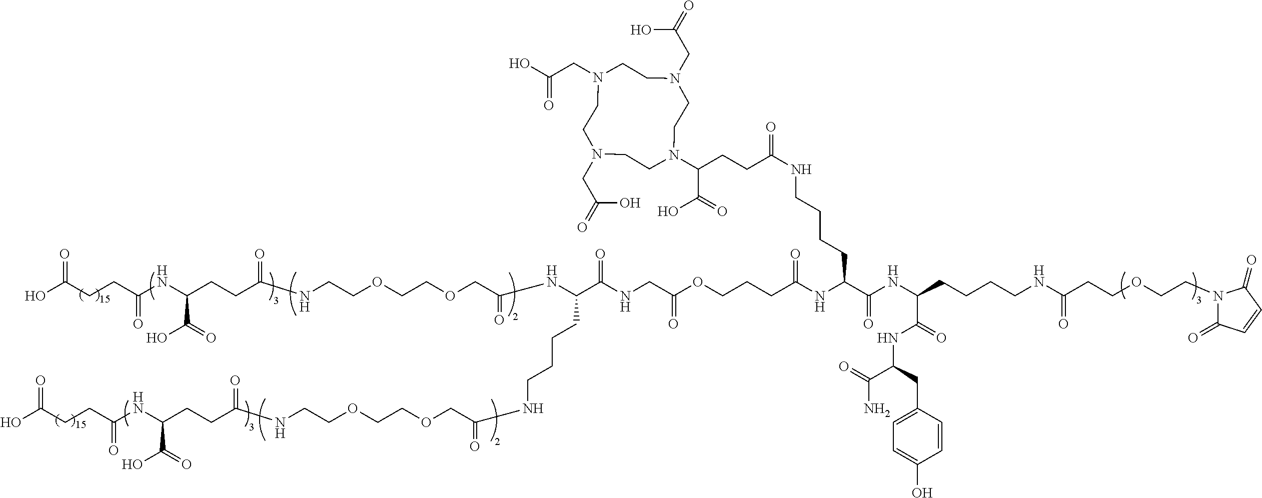 |
| | [HO-C18-gGlu-gGlu-gGlu-OEG-OEG]2-Lys-ESL1-Lys(DOTAGA)-Lys(PEG3-MAL)-Tyr-NH2 |
| L031 | 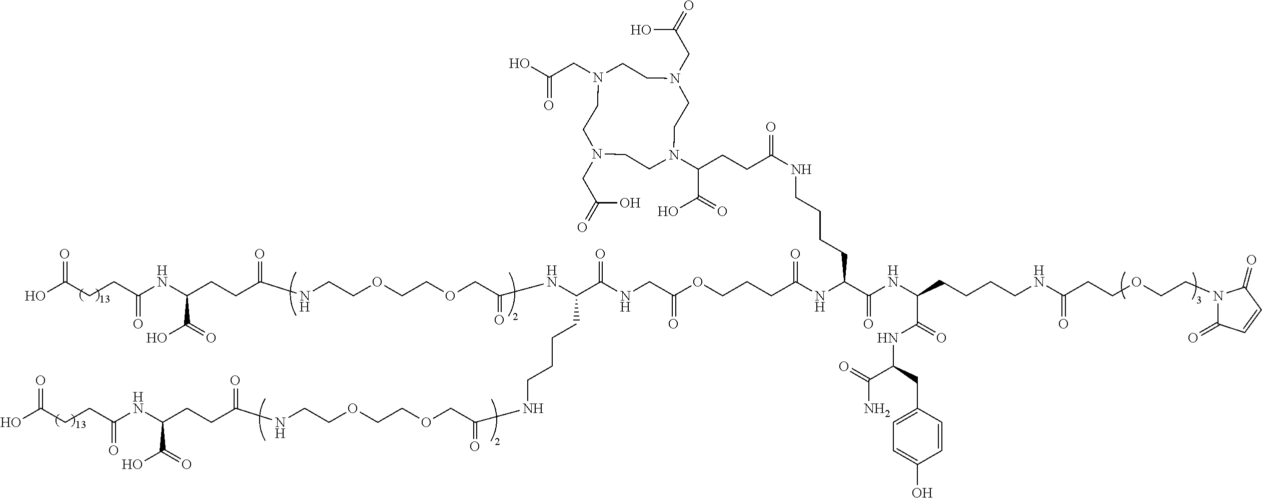 |
| | [HO-C16-gGlu-OEG-OEG]2-Lys-ESL1-Lys(DOTAGA)-Lys(PEG3-MAL)-Tyr-NH2 |
| L032 | 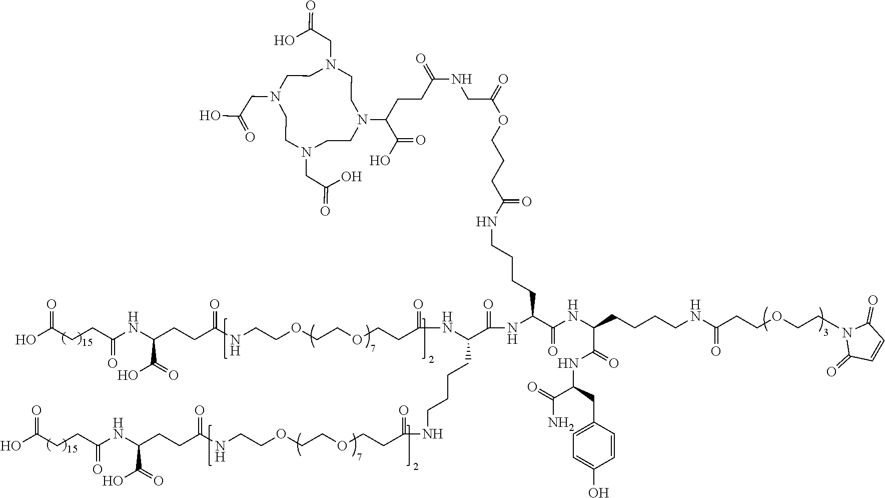 |
| | [HO-C18-gGlu-PEG8-PEG8]2-Lys-Lys(ESL1-DOTAGA)-Lys(PEG3-MAL)-Tyr-NH2 |
| L033 | 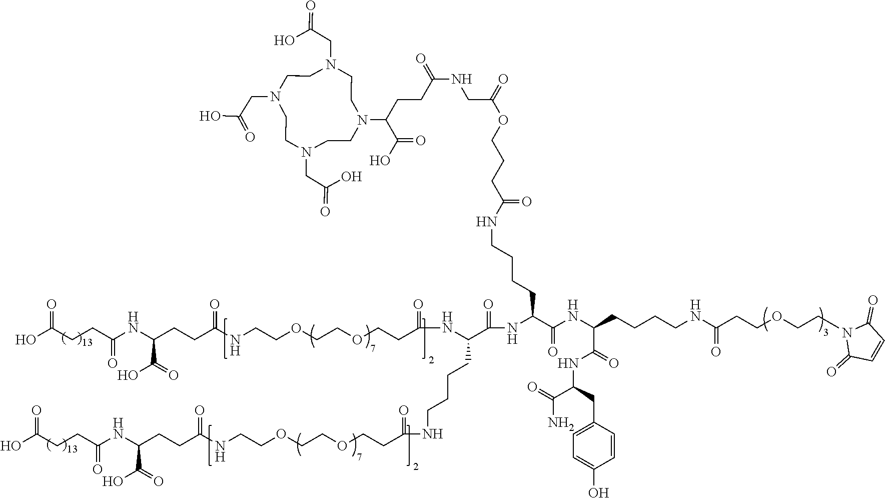 |
| | [HO-C16-gGlu-PEG8-PEG8]2-Lys-Lys(ESL1-DOTAGA)-Lys(PEG3-MAL)-Tyr-NH2 |
| L034 | 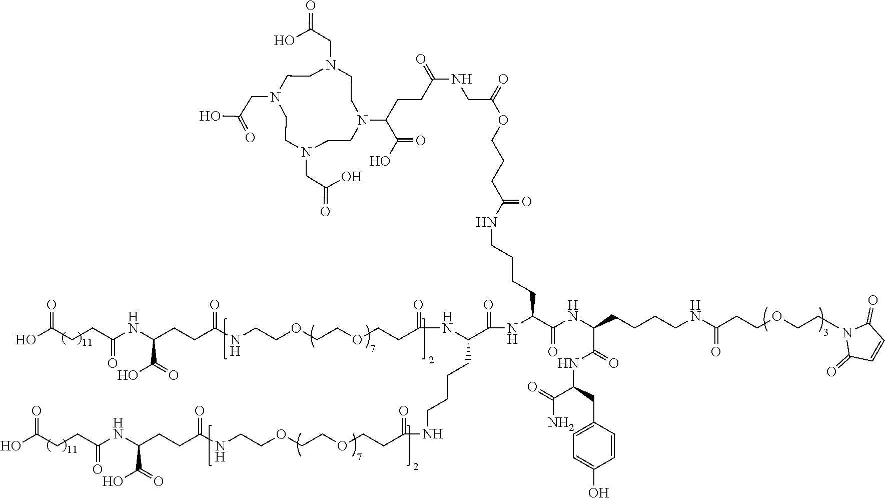 |
| | [HO-C14-gGlu-PEG8-PEG8]2-Lys-Lys(ESL1-DOTAGA)-Lys(PEG3-MAL)-Tyr-NH2 |
| L035 | 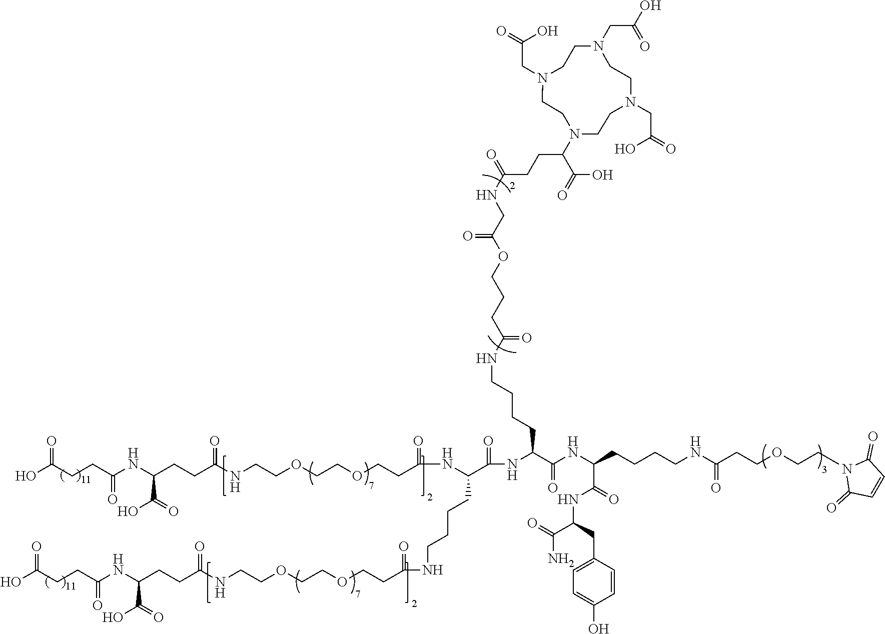 |
| | [HO-C18-gGlu-PEG8-PEG8]2-Lys-Lys(ESL1-ESL1-DOTAGA)-Lys(PEG3-MAL)-Tyr-NH2 |
| L036 | 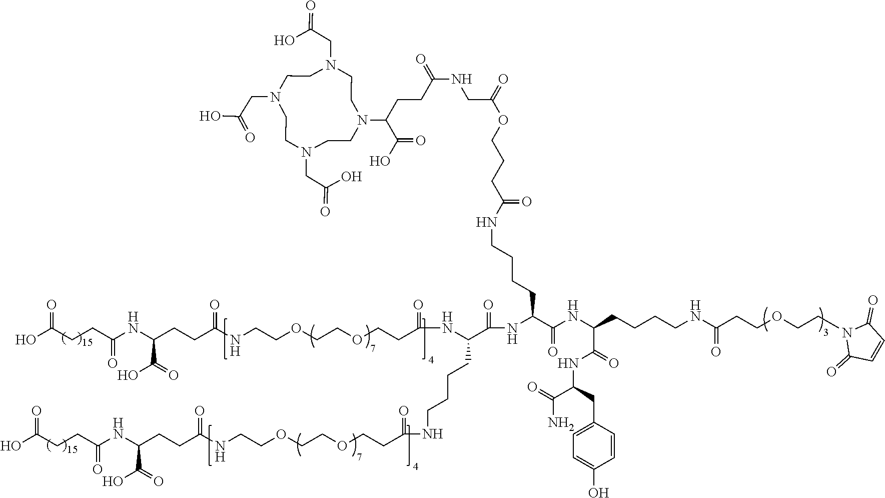 |
| | [HO-C18-gGlu-PEG8-PEG8-PEG8-PEG8]2-Lys-Lys(ESL1-DOTAGA)-Lys(PEG3-MAL)-Tyr-NH2 |
| L037 | 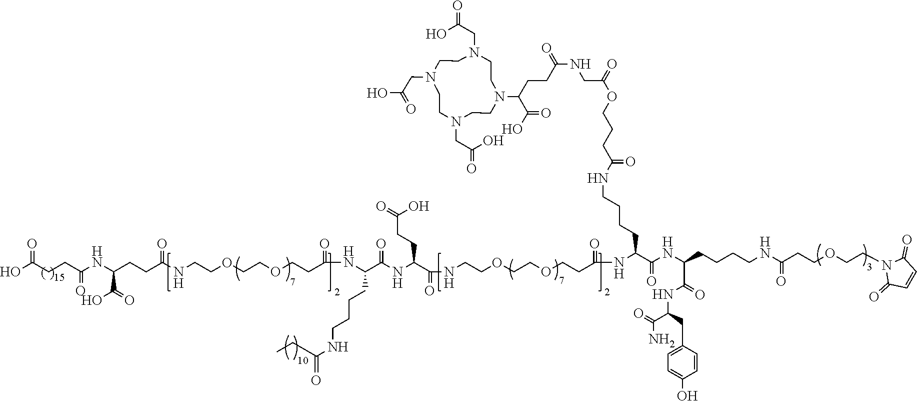 |
| | HO-C18-gGlu-PEG8-PEG8-Lys(C12)-Glu-PEG8-PEG8-Lys(ESL1-DOTAGA)-Lys(PEG3-MAL)-Tyr-NH2 |
| L038 | 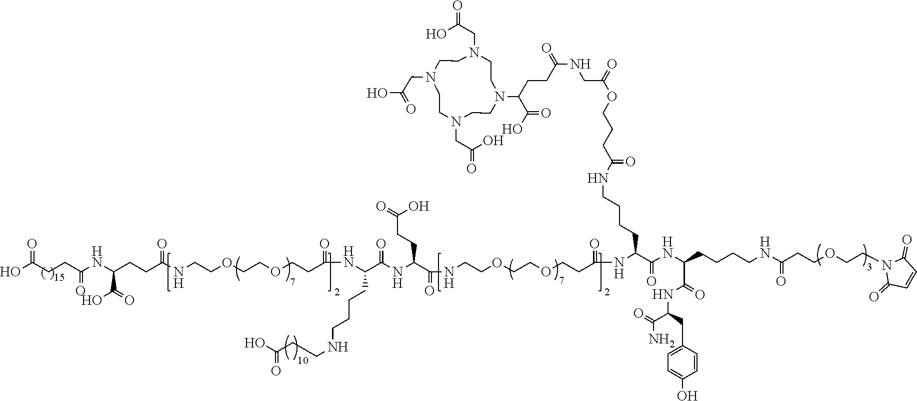 |
| | HO-C18-gGlu-PEG8-PEG8-Lys(C12-OH)-Glu-PEG8-PEG8-Lys(ESL1-DOTAGA)-Lys(PEG3-MAL)-Tyr-NH2 |
| L039 | 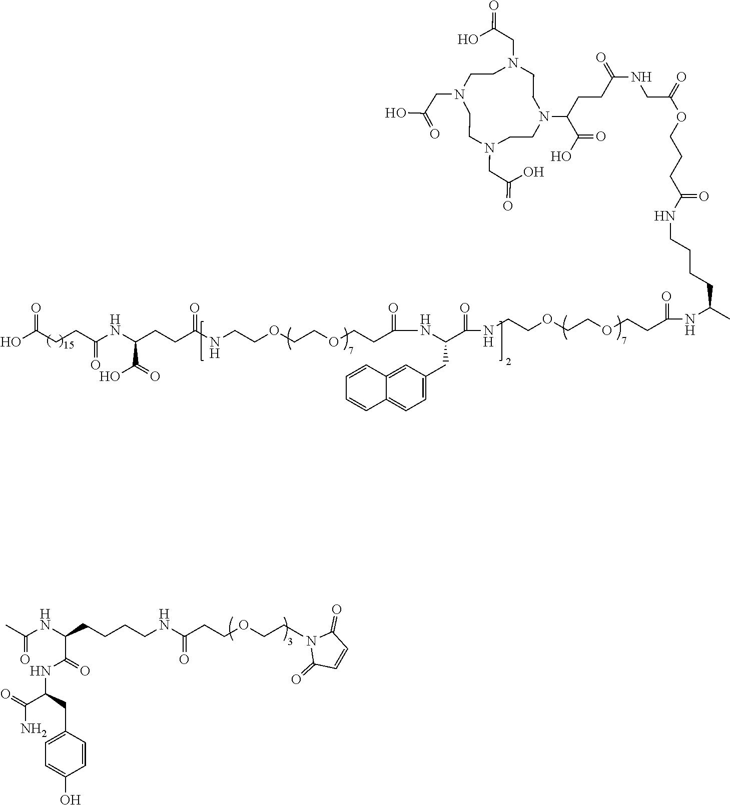 |
| | HO-C18-gGlu-PEG8-Nal-PEG8-Nal-PEG8-Lys(ESL1-DOTAGA)-Lys(PEG3-MAL)-Tyr-NH2 |
| L040 | 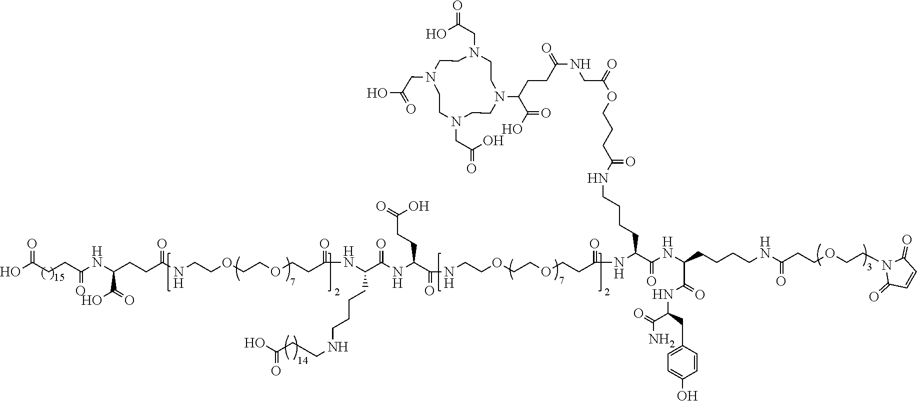 |
| | HO-C18-gGlu-PEG8-PEG8-Lys(HO-C16)-Glu-PEG8-PEG8-Lys(ESL1-DOTAGA)-Lys(PEG3-MAL)-Tyr-NH2 |
| L041 | 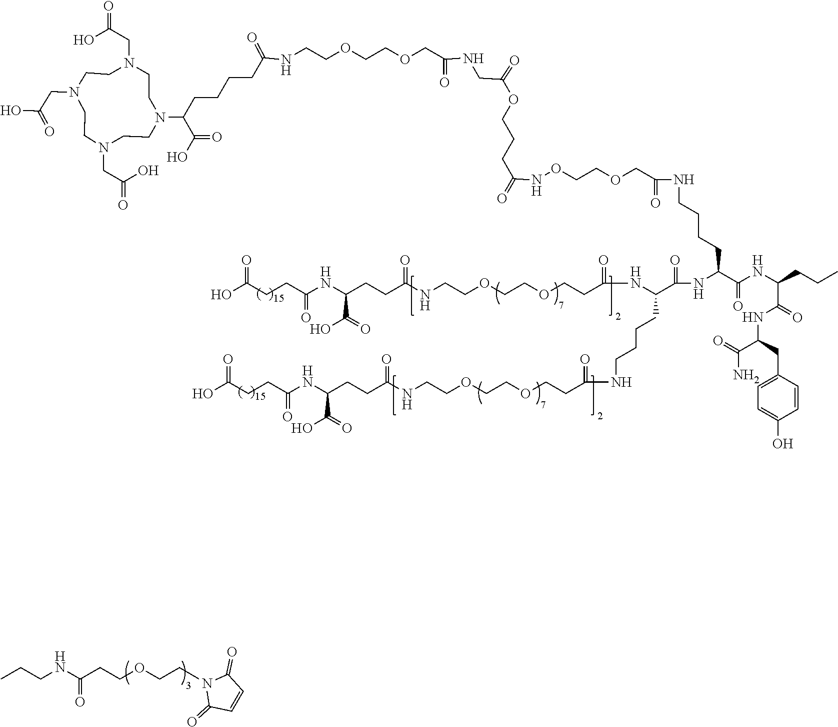 |
| | [HO-C18-gGlu-PEG8-PEG8-PEG8-PEG8]2-Lys-Lys(OEG-ESL1-OEG-DOTAGA)-Lys(PEG3-MAL)-Tyr-NH2 |
| L042 | 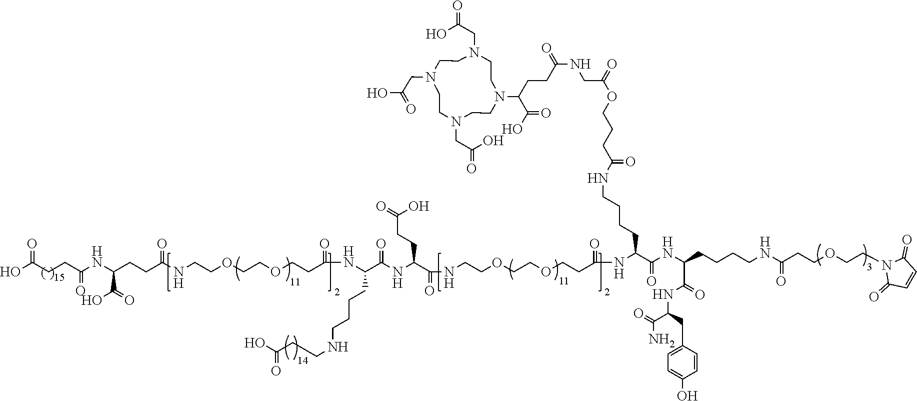 |
| | HO-C18-gGlu-PEG12-PEG12-Glu-Lys(C16)-Glu-PEG12-PEG12-Lys(ESL1-DOTAGA)-Lys(PEG3-MAL)-Tyr-NH2 |
| L043 | 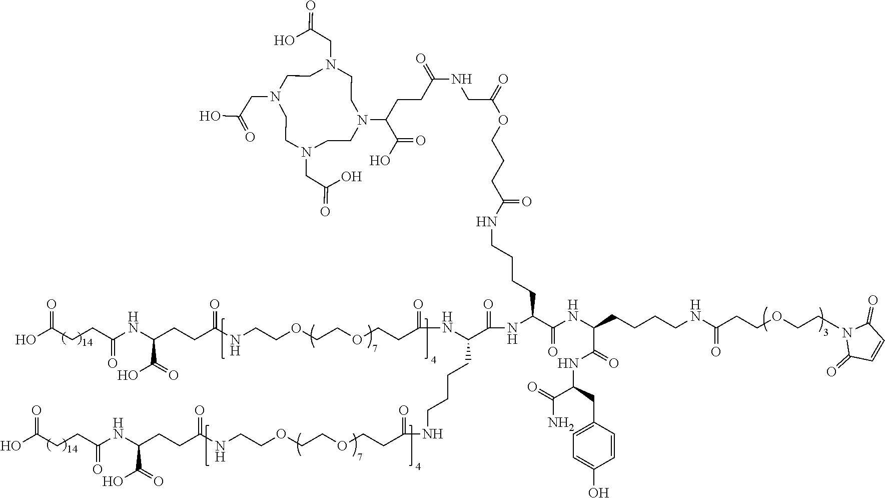 |
| | [HO-C17-gGlu-PEG8-PEG8-PEG8-PEG8]2-Lys-Lys(ESL1-DOTAGA)-Lys(PEG3-MAL)-Tyr-NH2 |
| L044 | 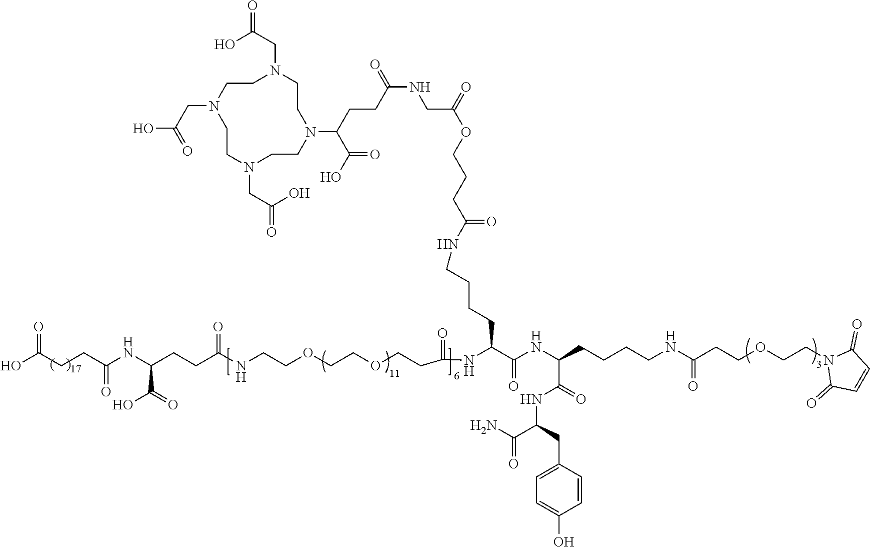 |
| | HO-C20-gGlu-PEG12-PEG12-PEG12-PEG12-PEG12-PEG12-Lys(ESL1-DOTAGA)-Lys(PEG3-MAL)-Tyr-NH2 |
| L045 | 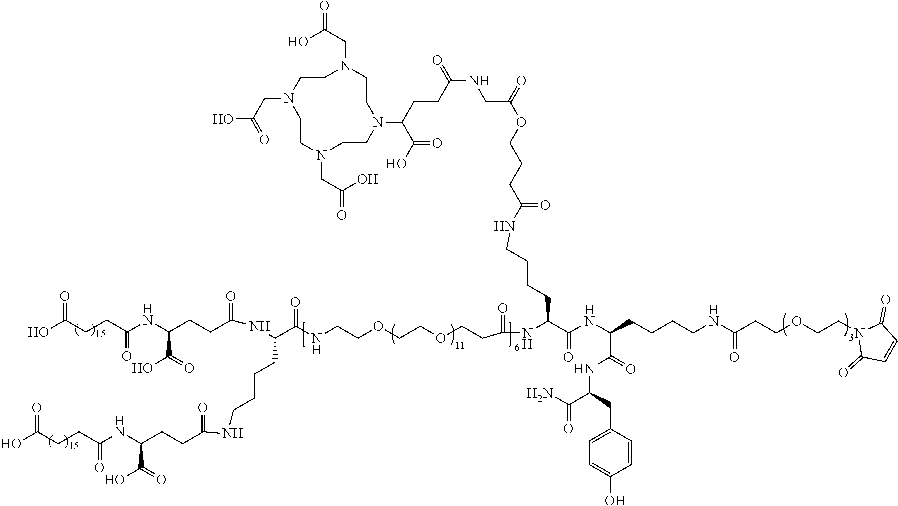 |
| | [HO-C18-gGlu]2-Lys-PEG12-PEG12-PEG12-PEG12-PEG12-PEG12-Lys(ESL1-DOTAGA)-Lys(PEG3-MAL)-Tyr-NH2 |
| L046 | 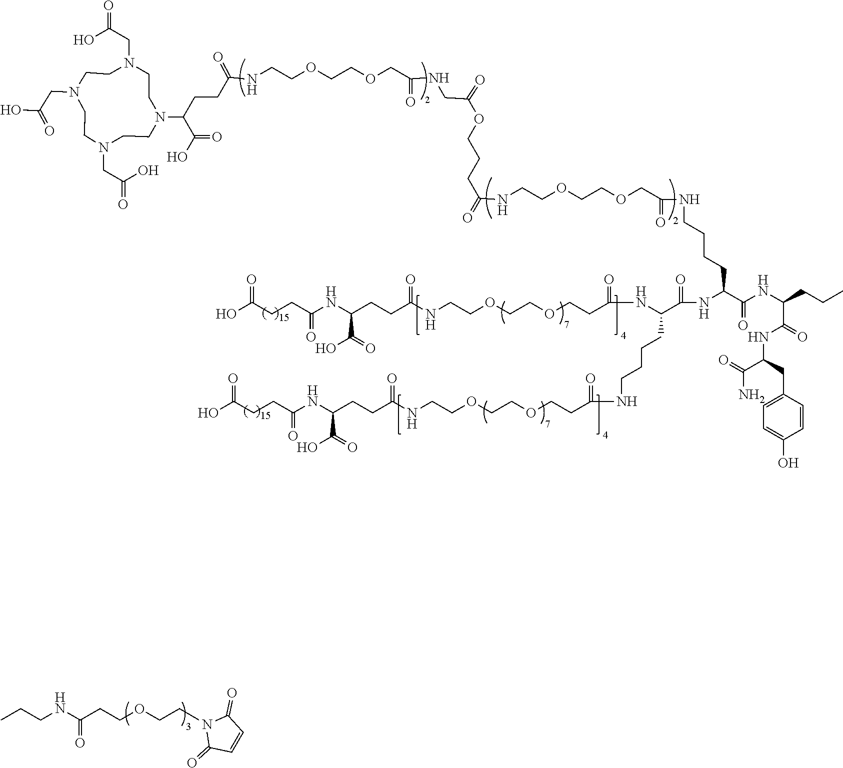 |
| | [HO-C18-gGlu-PEG8-PEG8-PEG8-PEG8]2-Lys-Lys(OEG-OEG-ESL1-OEG-OEG-DOTAGA)-Lys(PEG3-MAL)-Tyr-NH2 |
| L047 | 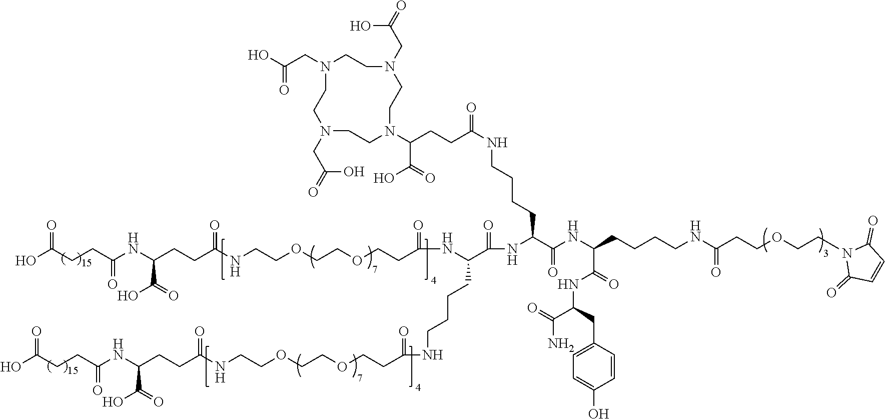 |
| | [HO-C18-gGlu-PEG8-PEG8-PEG8-PEG8]2-Lys-Lys(DOTAGA)-Lys(PEG3-MAL)-Tyr-NH2 |
| L048 | 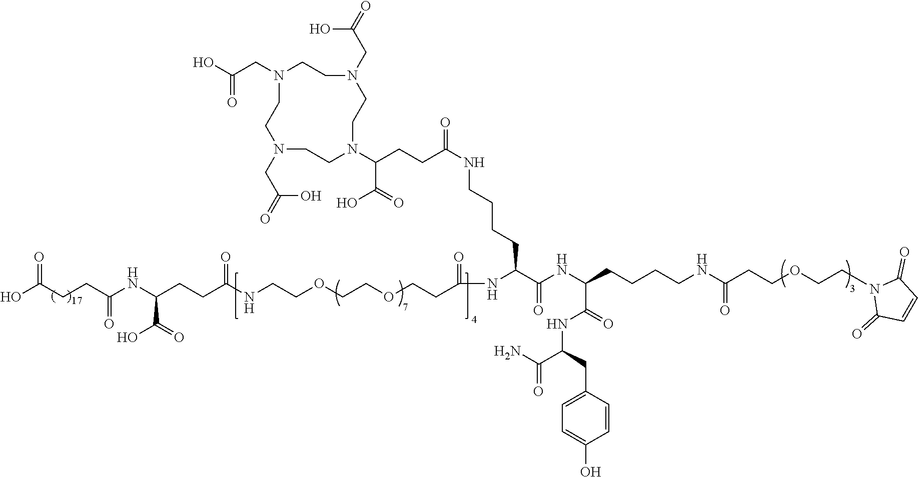 |
| | HO-C20-gGlu-PEG8-PEG8-PEG8-PEG8-Lys(DOTAGA)-Lys(PEG3-MAL)-Tyr-NH2 |
| L049 | 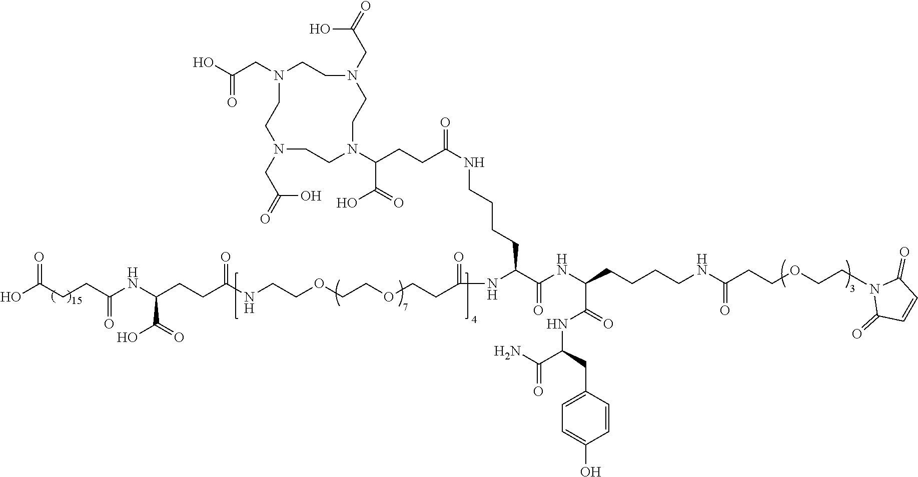 |
| | HO-C18-gGlu-PEG8-PEG8-PEG8-PEG8-Lys(DOTAGA)-Lys(PEG3-MAL)-Tyr-NH2 |
| L050 | 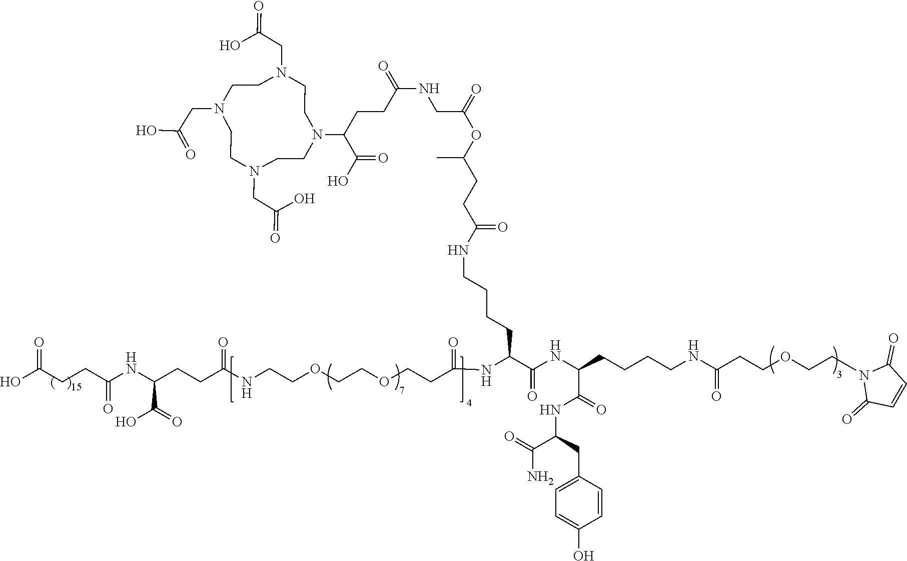 |
| | HO-C18-gGlu-PEG8-PEG8-PEG8-PEG8-Lys(4hPA-Gly-DOTAGA)-Lys(PEG3-MAL)-Tyr-NH2 |
| L051 | 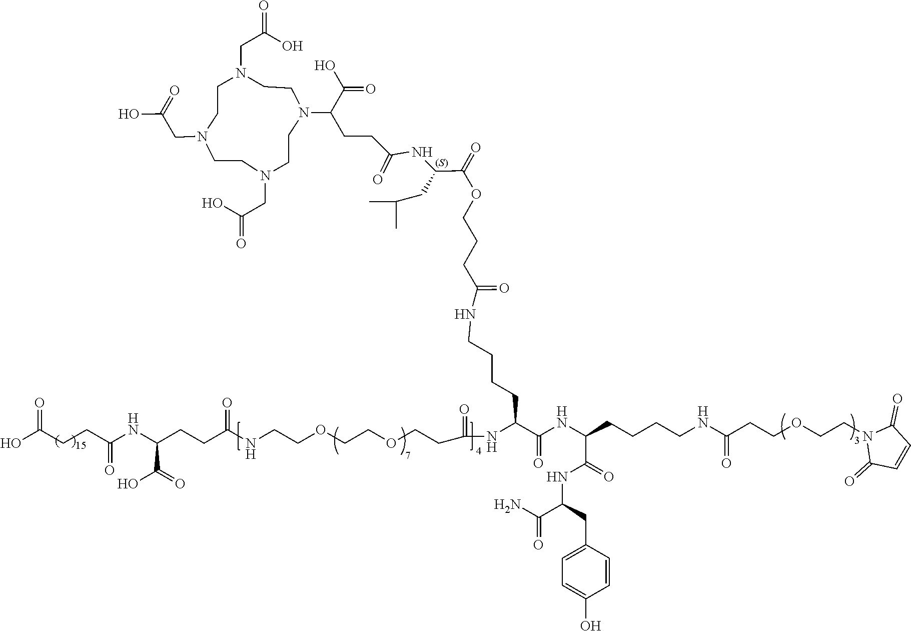 |
| | HO-C18-gGlu-PEG8-PEG8-PEG8-PEG8-Lys(4hBA-Leu-DOTAGA)-Lys(PEG3-MAL)-Tyr-NH2 |
| L052 | 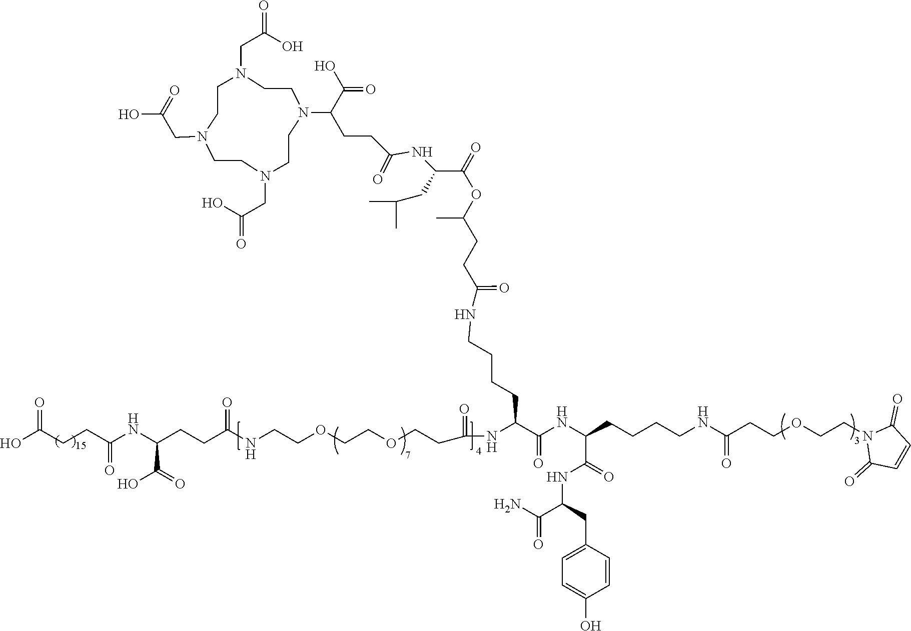 |
| | HO-C18-gGlu-PEG8-PEG8-PEG8-PEG8-Lys(4hPA-Leu-DOTAGA)-Lys(PEG3-MAL)-Tyr-NH2 |
| L053 | 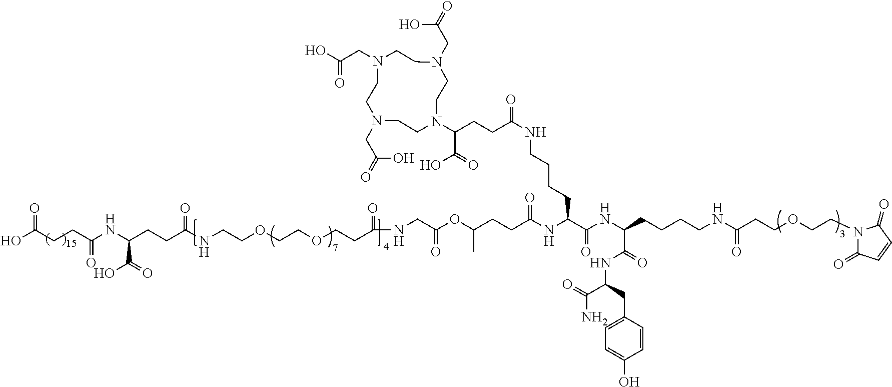 |
| | HO-C18-gGlu-PEG8-PEG8-PEG8-PEG8-Gly-4hPA-Lys(DOTAGA)-Lys(PEG3-MAL)-Tyr-NH2 |
| L054 | 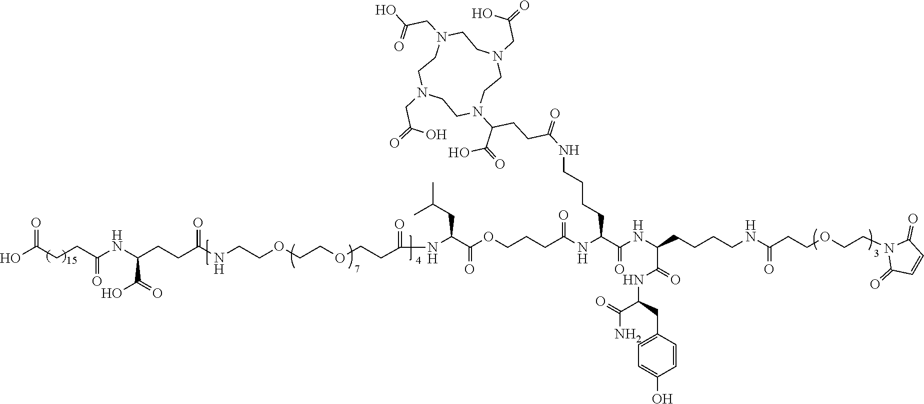 |
| | HO-C18-gGlu-PEG8-PEG8-PEG8-PEG8-Leu-4hBA-Lys(DOTAGA)-Lys(PEG3-MAL)-Tyr-NH2 |
| L055 | 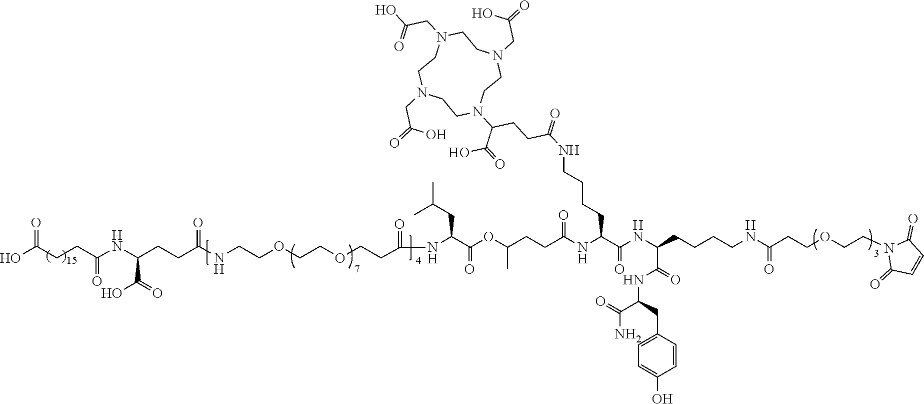 |
| | HO-C18-gGlu-PEG8-PEG8-PEG8-PEG8-Leu-4hPA-Lys(DOTAGA)-Lys(PEG3-MAL)-Tyr-NH2 |
| L056 | 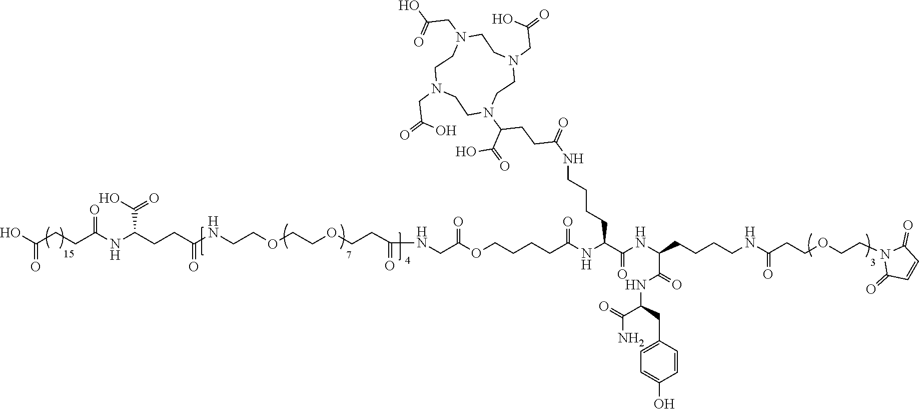 |
| | HO-C18-gGlu-PEG8-PEG8-PEG8-PEG8-Gly-5hPA-Lys(DOTAGA)-Lys(PEG3-MAL)-Tyr-NH2 |
| L057 | 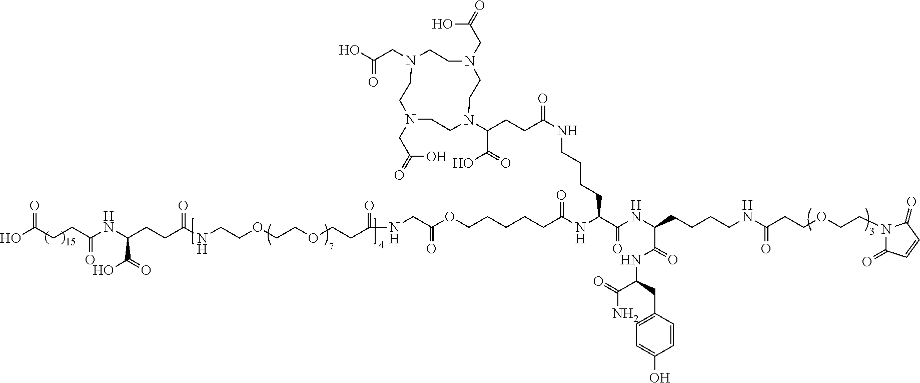 |
| | HO-C18-gGlu-PEG8-PEG8-PEG8-PEG8-Gly-6hHA-Lys(DOTAGA)-Lys(PEG3-MAL)-Tyr-NH2 |
| L058 | 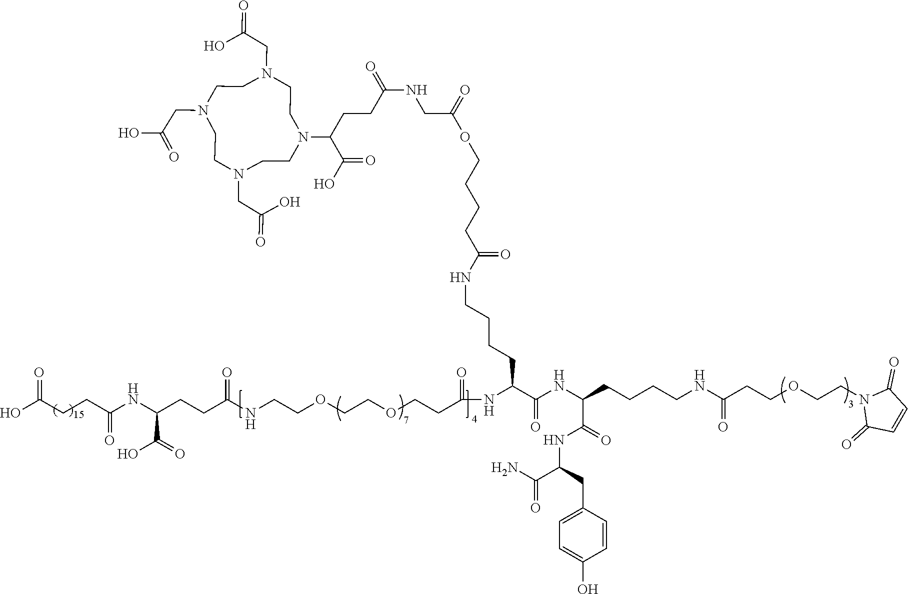 |
| | HO-C18-gGlu-PEG8-PEG8-PEG8-PEG8-Lys(5hPA-Gly-DOTAGA)-Lys(PEG3-MAL)-Tyr-NH2 |
| L059 | 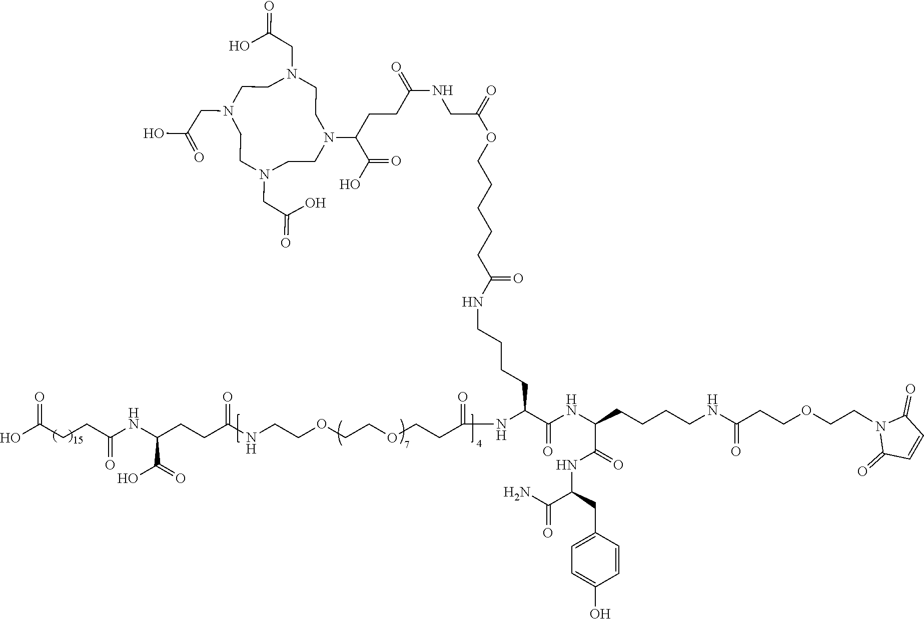 |
| | HO-C18-gGlu-PEG8-PEG8-PEG8-PEG8-Lys(6hPA-Gly-DOTAGA)-Lys(PEG3-MAL)-Tyr-NH2 |
| L060 | 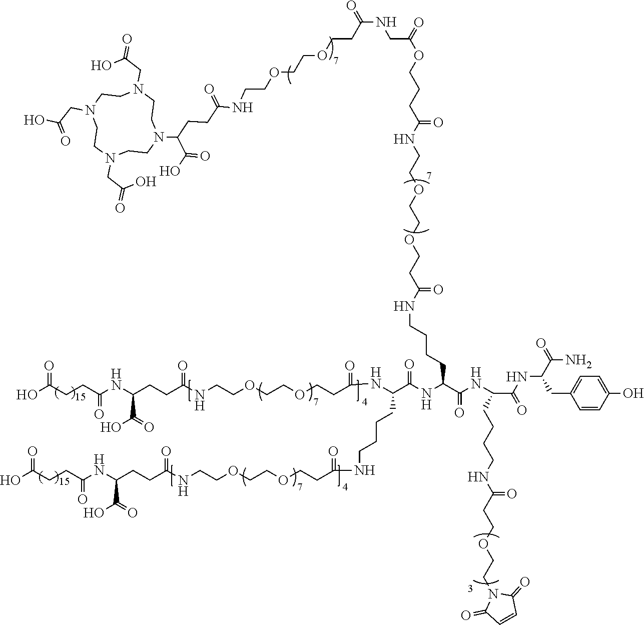 |
| | [HO-C18-gGlu-PEG8-PEG8-PEG8-PEG8]2-Lys-Lys(PEG8ESL1-PEG8-DOTAGA)-Lys(PEG3-MAL)-Tyr-NH2 |
| L061 | 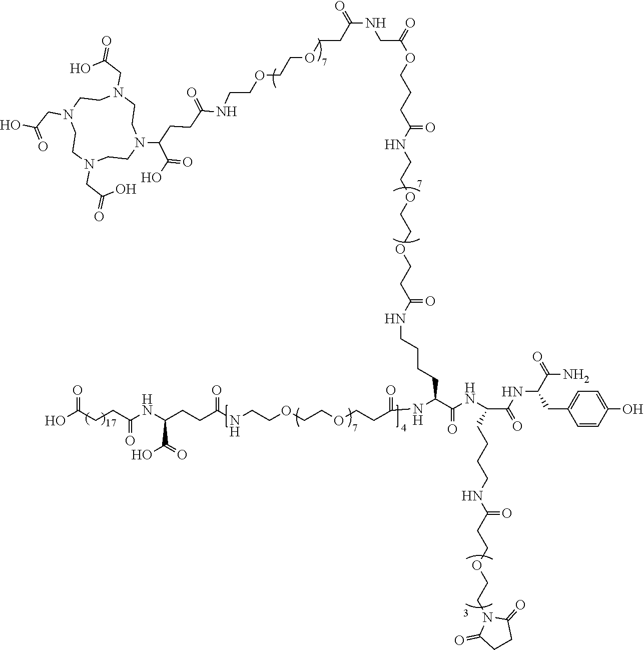 |
| | HO-C20-gGlu-PEG8-PEG8-PEG8-PEG8-Lys(PEG8-ESL1-PEG8-DOTAGA)-Lys(PEG3-MAL)-Tyr-NH2 |
| L062 | 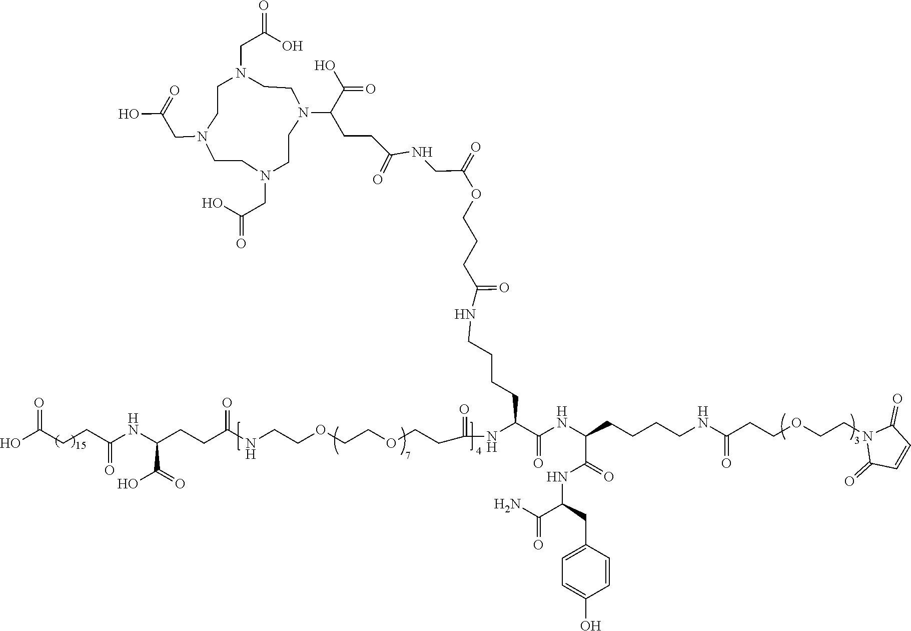 |
| | HO-C18-gGlu-PEG8-PEG8-PEG8-PEG8-Lys(ESL1-DOTAGA)-Lys(PEG3-MAL)-Tyr-NH2 |
| L063 | 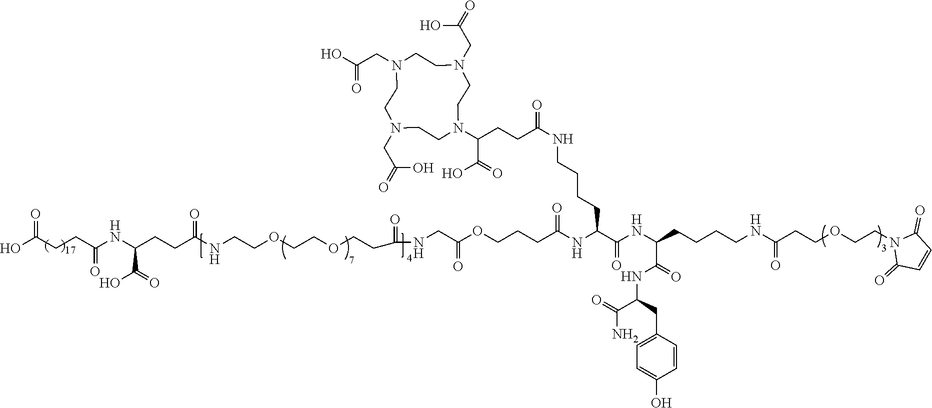 |
| | HO-C20-gGlu-PEG8-PEG8-PEG8-PEG8-Lys(DOTAGA)-Lys(PEG3-MAL)-Tyr-NH2 |
| L064 | 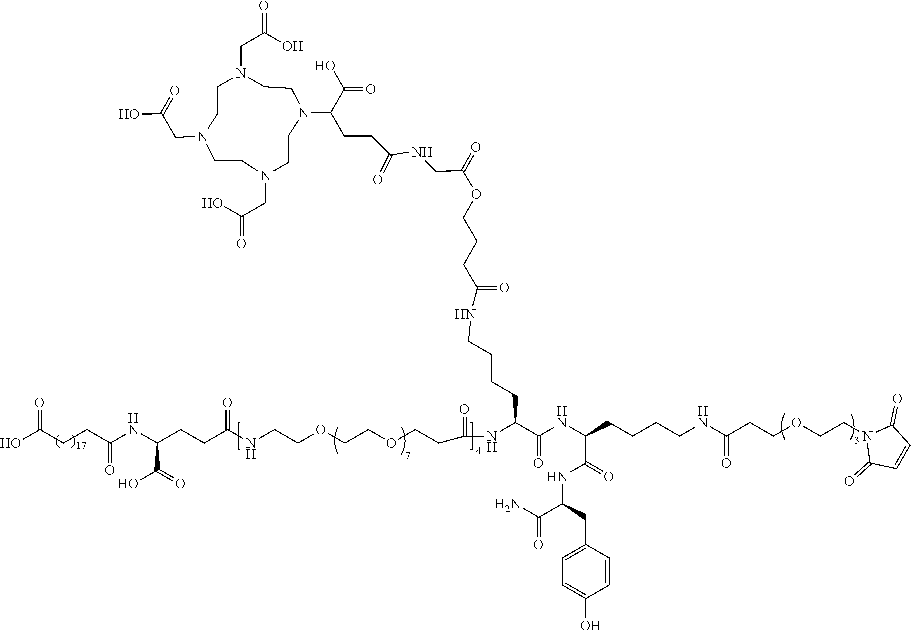 |
| | HO-C20-gGlu-PEG8-PEG8-PEG8-PEG8-Lys(ESL1-DOTAGA)-Lys(PEG3-MAL)-Tyr-NH2 |
| L065 | 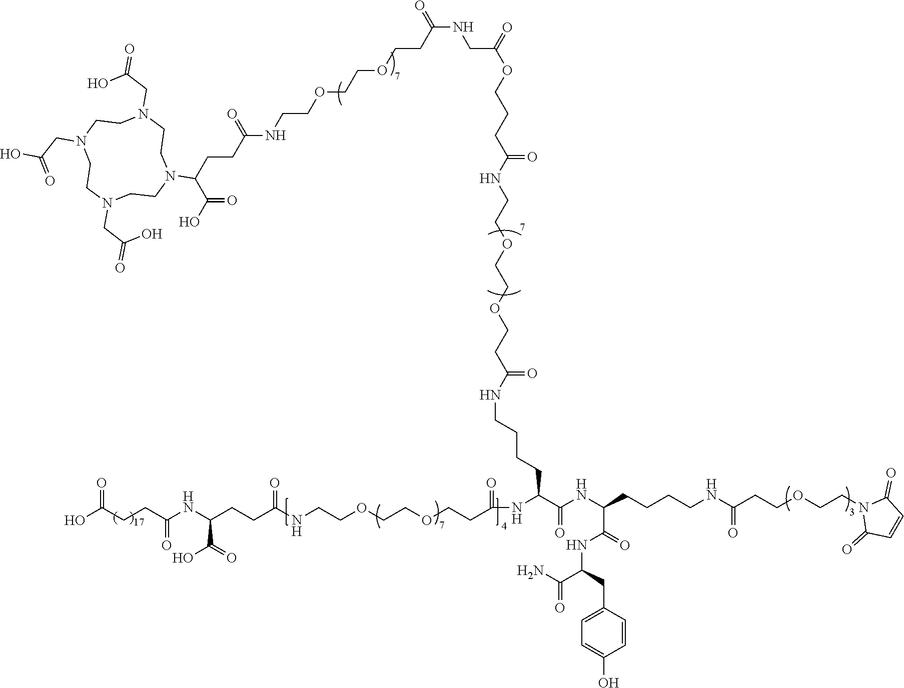 |
| | HO-C20-gGlu-PEG8-PEG8-PEG8-PEG8-Lys(PEG8-ESL1-PEG8-DOTAGA)-Lys(PEG3-MAL)-Tyr-NH2 |
| L066 | 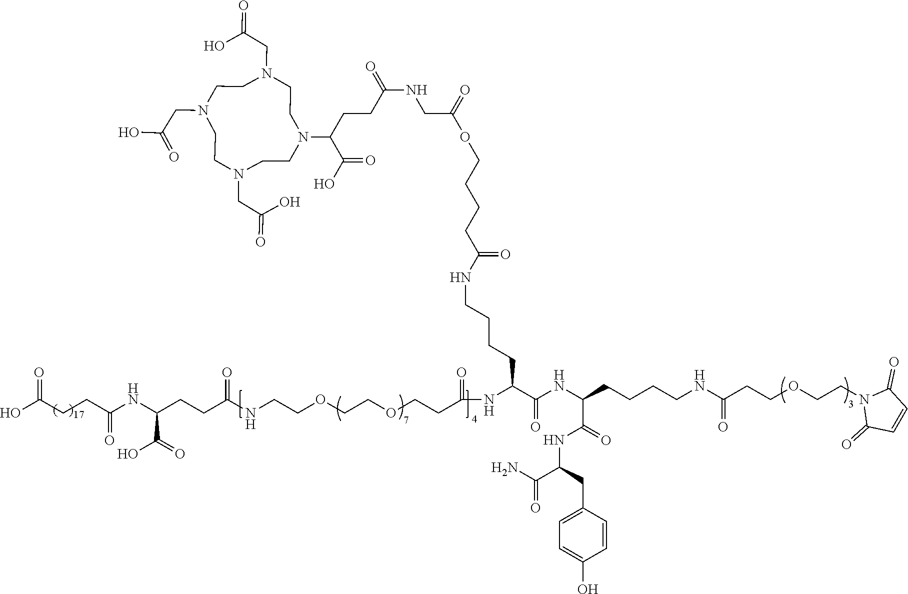 |
| | HO-C20-gGlu-PEG8-PEG8-PEG8-PEG8-Lys(5hPA-Gly-DOTAGA)-Lys(PEG3-MAL)-Tyr-NH2 |
| L067 | 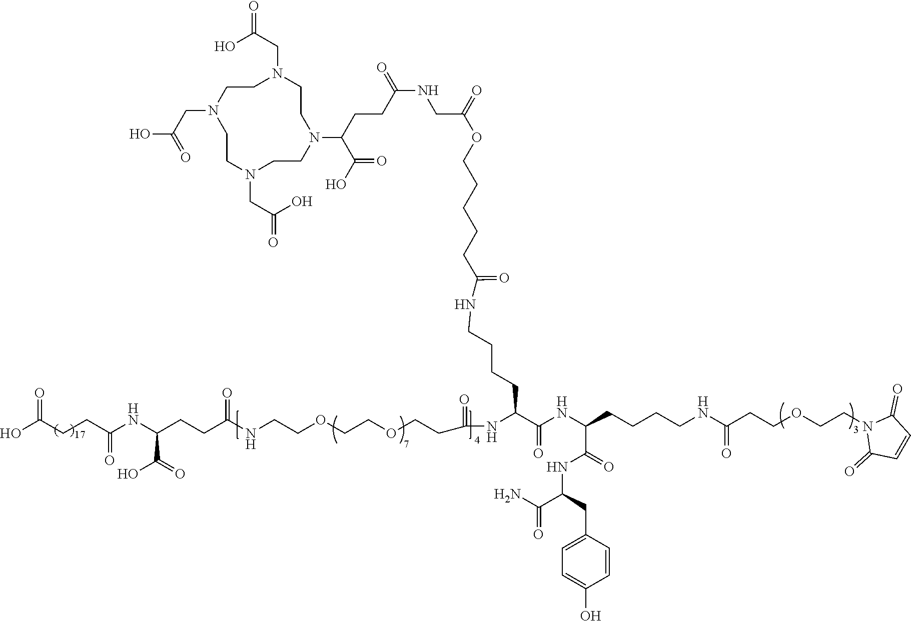 |
| | HO-C20-gGlu-PEG8-PEG8-PEG8-PEG8-Lys(6hHA-Gly-DOTAGA)-Lys(PEG3-MAL)-Tyr-NH2 |
| L068 | 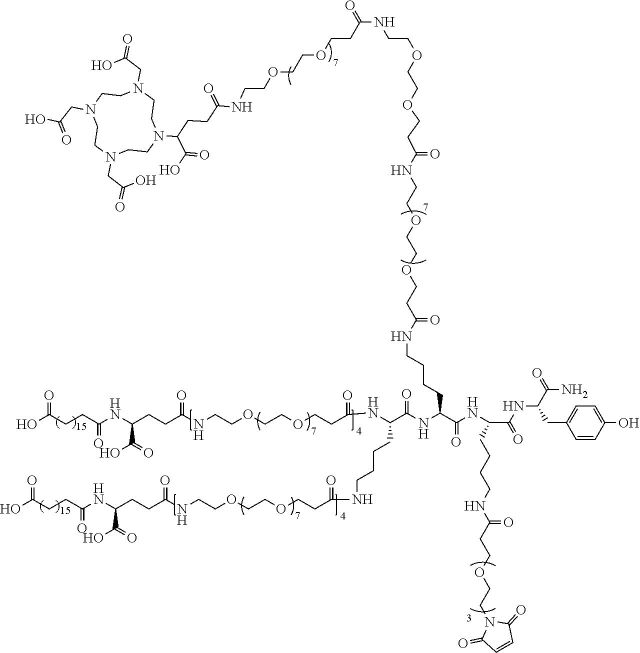 |
| | [HO-C18-gGlu-PEG8-PEG8-PEG8-PEG8]2-Lys-Lys(PEG8-OEG-PEG8-DOTAGA)-Lys(PEG3-MAL)-Tyr-NH2 |
| L069 | 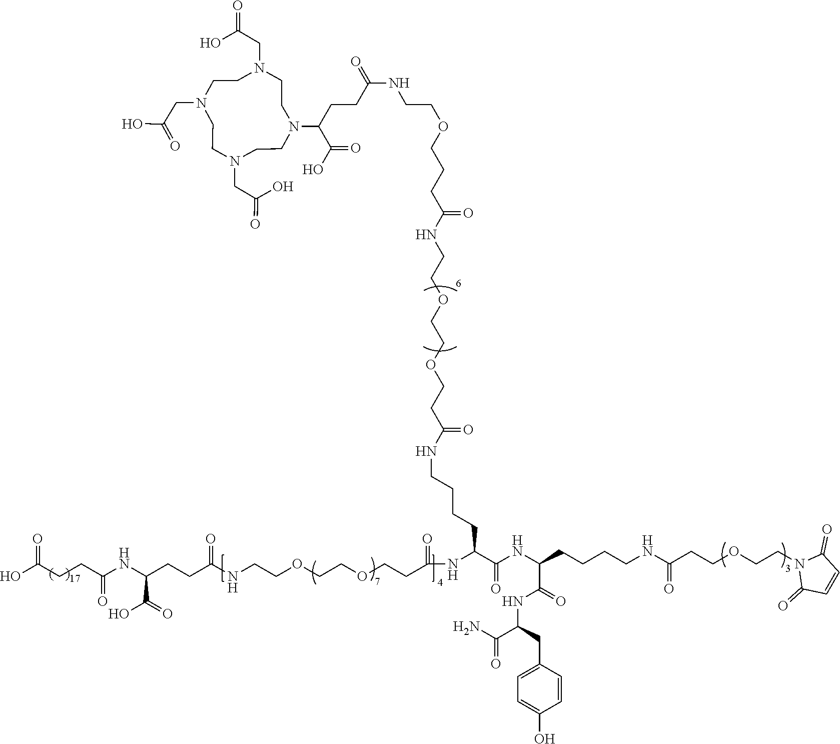 |
| | HO-C20-gGlu-PEG8-PEG8-PEG8-PEG8-Lys(PEG6-ESL1-DOTAGA)-Lys(PEG3-MAL)-Tyr-NH2 |
| L070 | 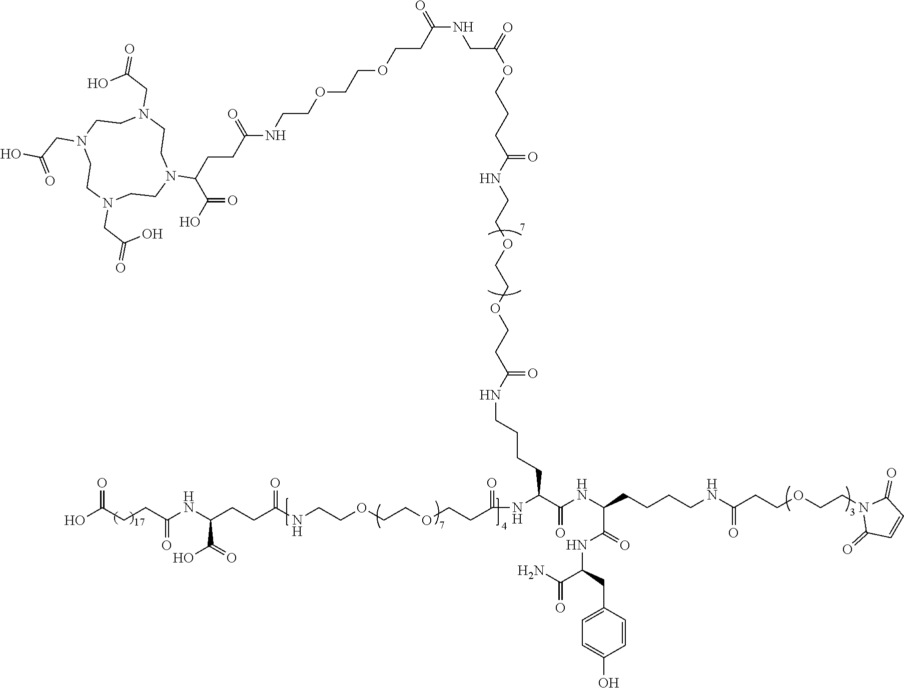 |
| | HO-C20-gGlu-PEG8-PEG8-PEG8-PEG8-Lys(PEG8-ESL1-OEG-DOTAGA)-Lys(PEG3-MAL)-Tyr-NH2 |
| L071 | 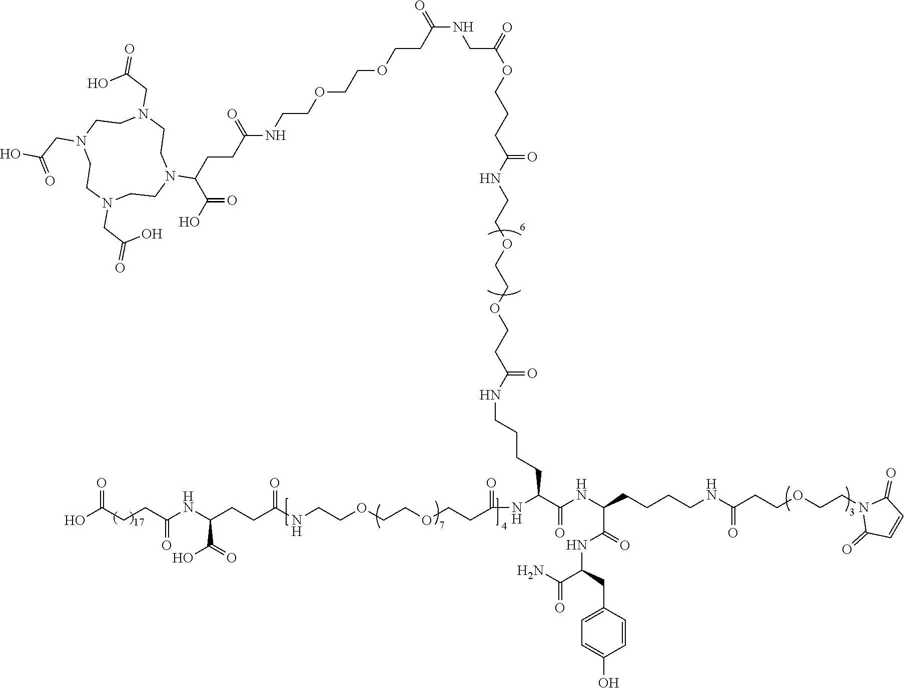 |
| | HO-C20-gGlu-PEG8-PEG8-PEG8-PEG8-Lys(PEG6-ESL1-OEG-DOTAGA)-Lys(PEG3-MAL)-Tyr-NH2 |
| L072 | 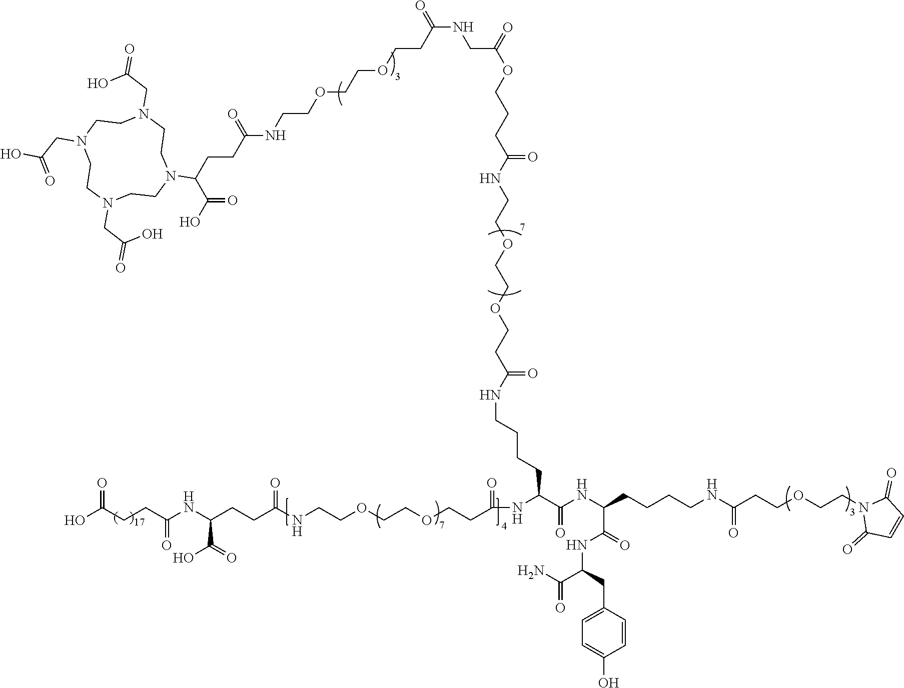 |
| | HO-C20-gGlu-PEG8-PEG8-PEG8-PEG8-Lys(PEG8-ESL1-PEG4-DOTAGA)-Lys(PEG3-MAL)-Tyr-NH2 |
| L073 | 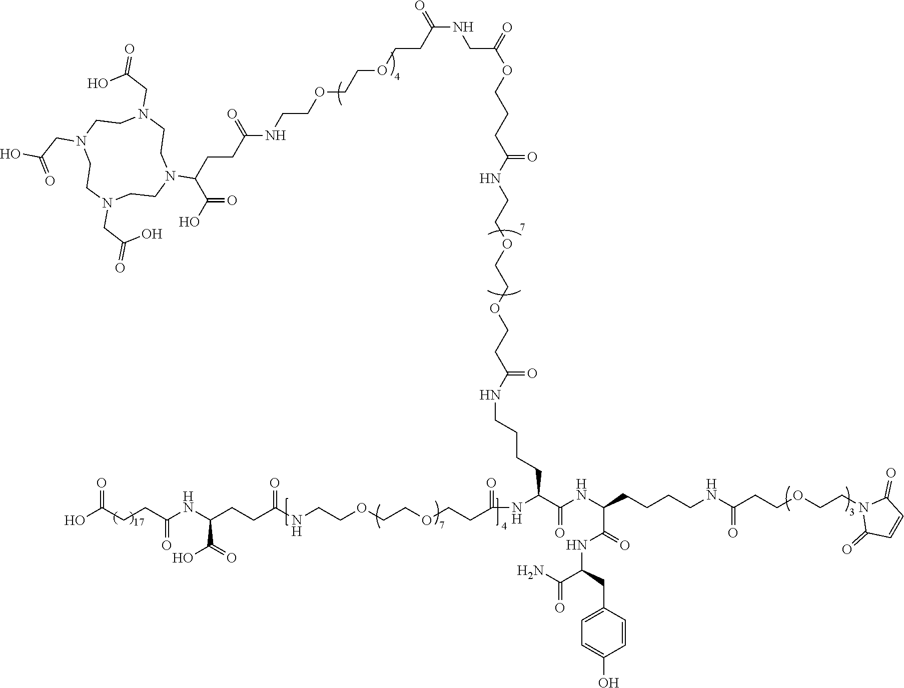 |
| | HO-C20-gGlu-PEG8-PEG8-PEG8-PEG8-Lys(PEG8-ESL1-PEG5-DOTAGA)-Lys(PEG3-MAL)-Tyr-NH2 |
| L074 | 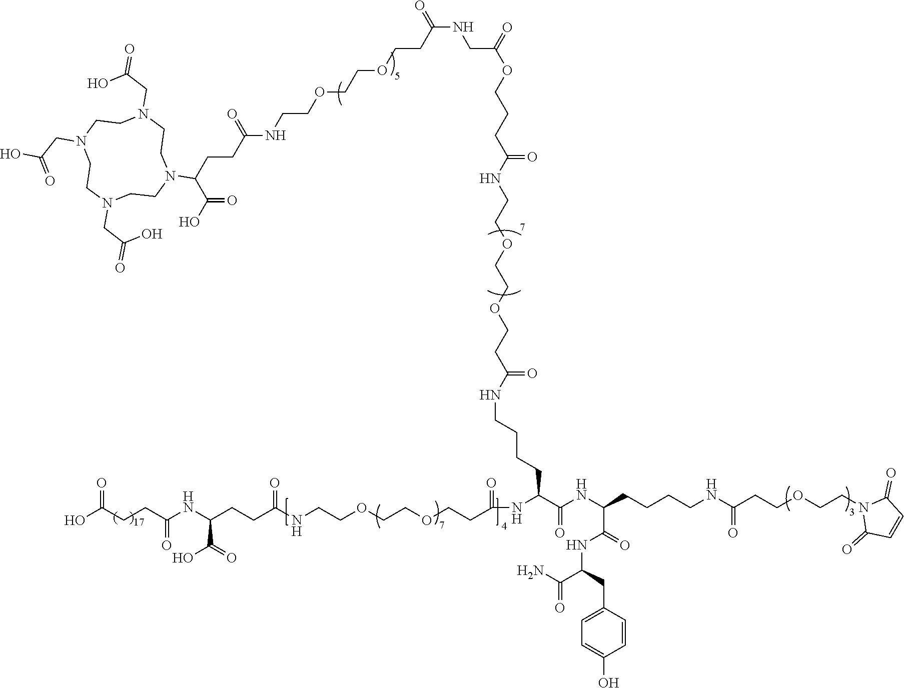 |
| | HO-C20-gGlu-PEG8-PEG8-PEG8-PEG8-Lys(PEG8-ESL1-PEG6-DOTAGA)-Lys(PEG3-MAL)-Tyr-NH2 |
| L075 | 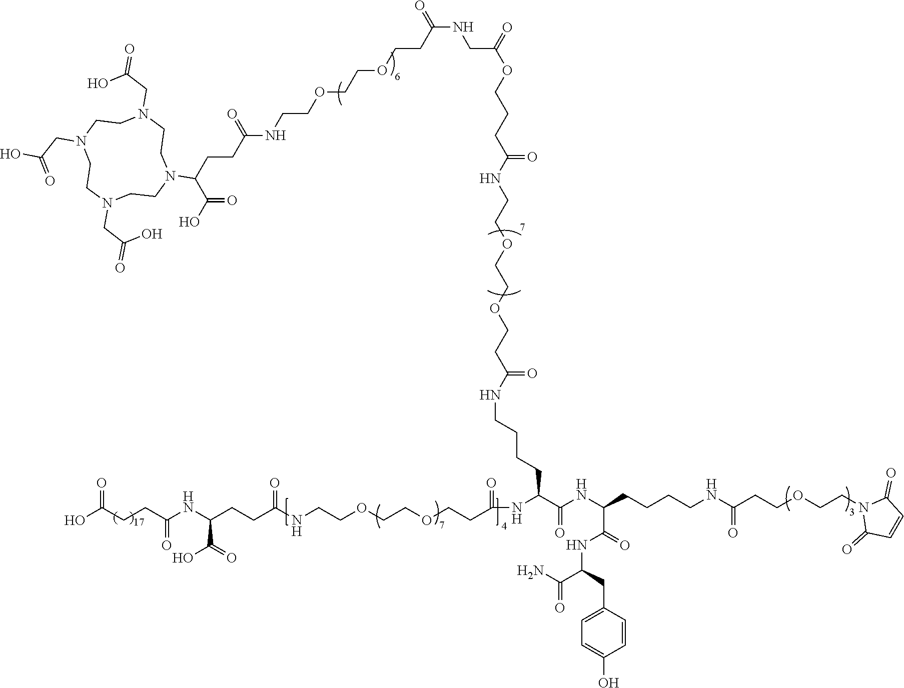 |
| | HO-C20-gGlu-PEG8-PEG8-PEG8-PEG8-Lys(PEG8-ESL1-PEG7-DOTAGA)-Lys(PEG3-MAL)-Tyr-NH2 |
| L076 | 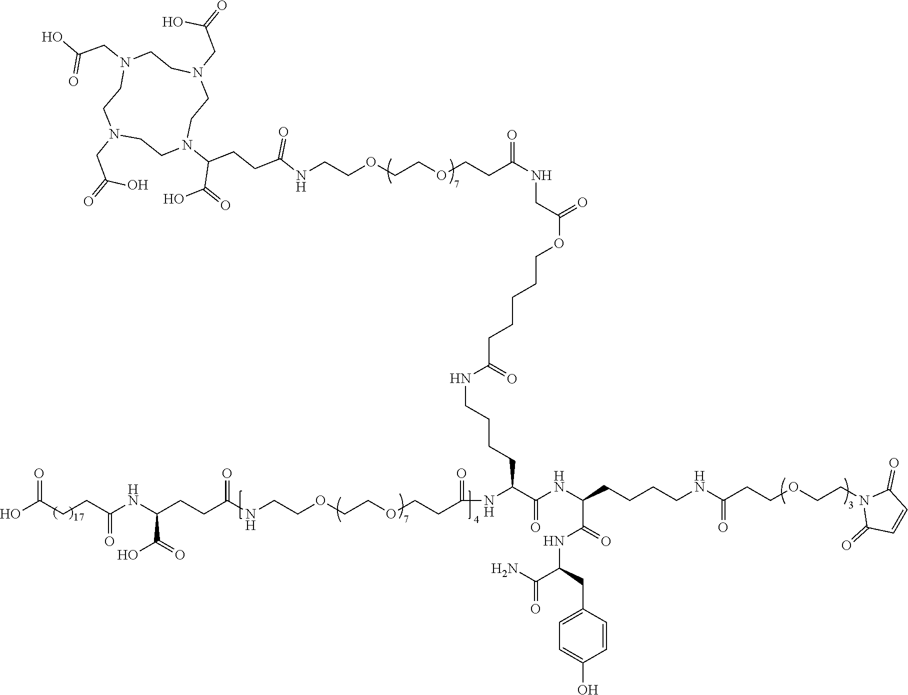 |
| | HO-C20-gGlu-PEG8-PEG8-PEG8-PEG8-Lys(6hHA-Gly-PEG8-DOTAGA)-Lys(PEG3-MAL)-Tyr-NH2 |
| L077 | 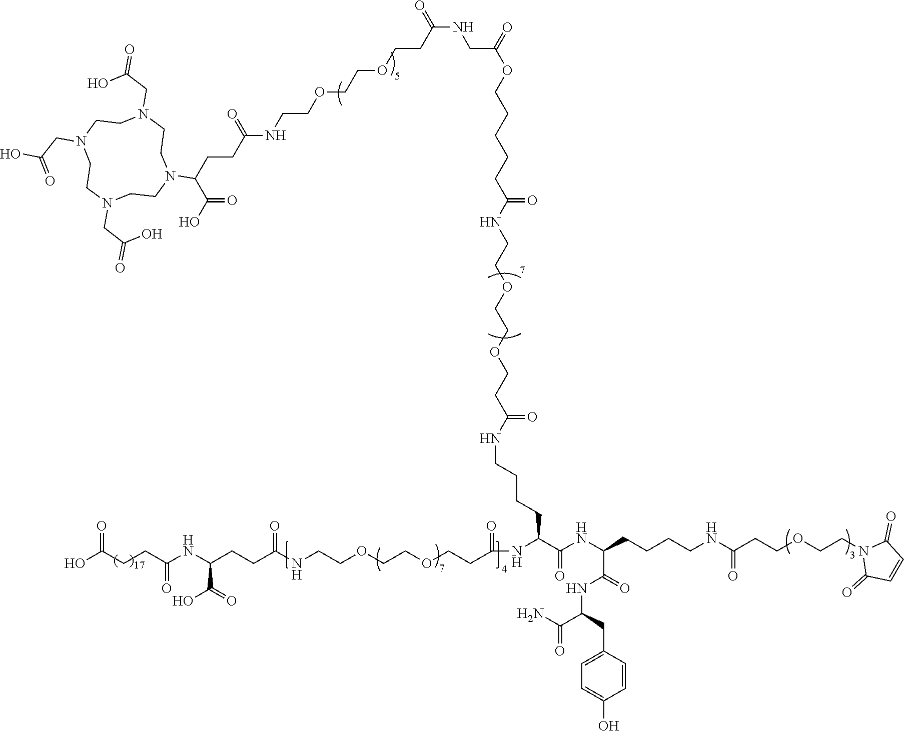 |
| | HO-C20-gGlu-PEG8-PEG8-PEG8-PEG8-Lys(PEG8-6hHA-Gly-PEG6-DOTAGA)-Lys(PEG3-MAL)-Tyr-NH2 |
| L078 | 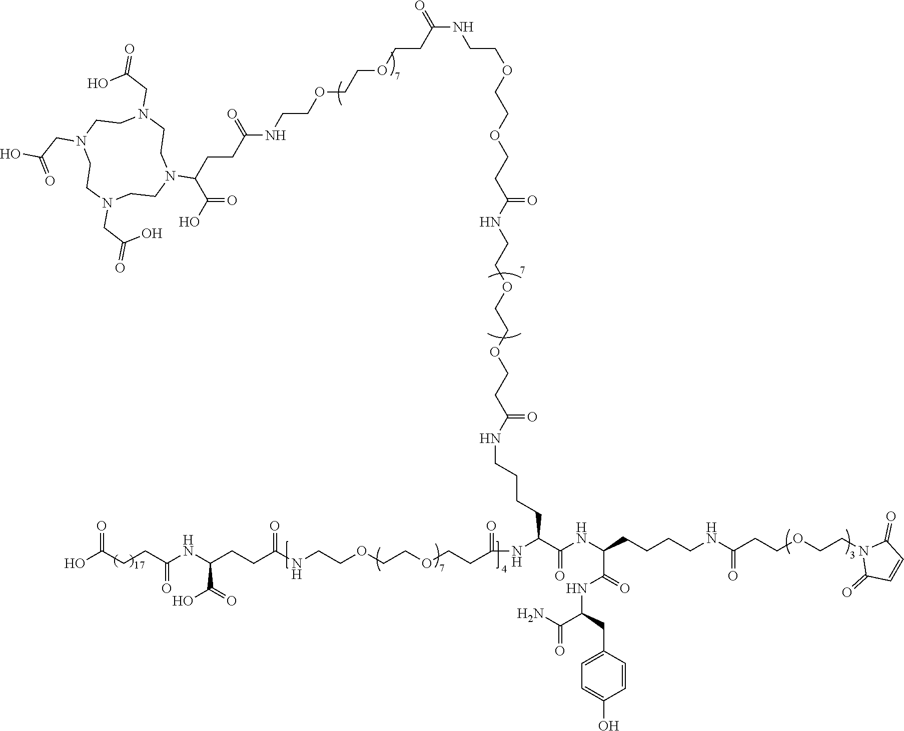 |
| | HO-C20-gGlu-PEG8-PEG8-PEG8-PEG8-Lys(PEG8-OEG-PEG8-DOTAGA)-Lys(PEG3-MAL)-Tyr-NH2 |
| L079 | 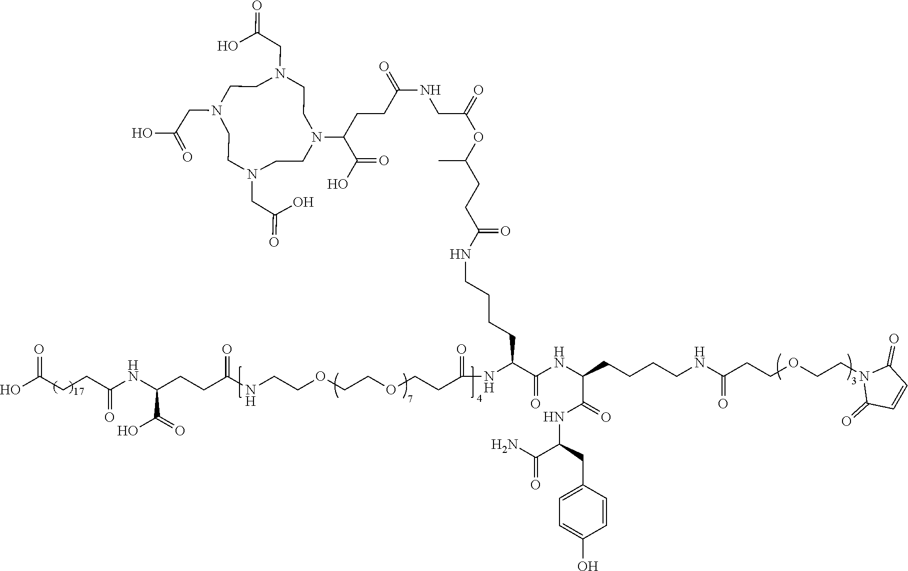 |
| | HO-C20-gGlu-PEG8-PEG8-PEG8-PEG8-Lys(4hPA-Gly-DOTAGA)-Lys(PEG3-MAL)-Tyr-NH2 |
| L080 | 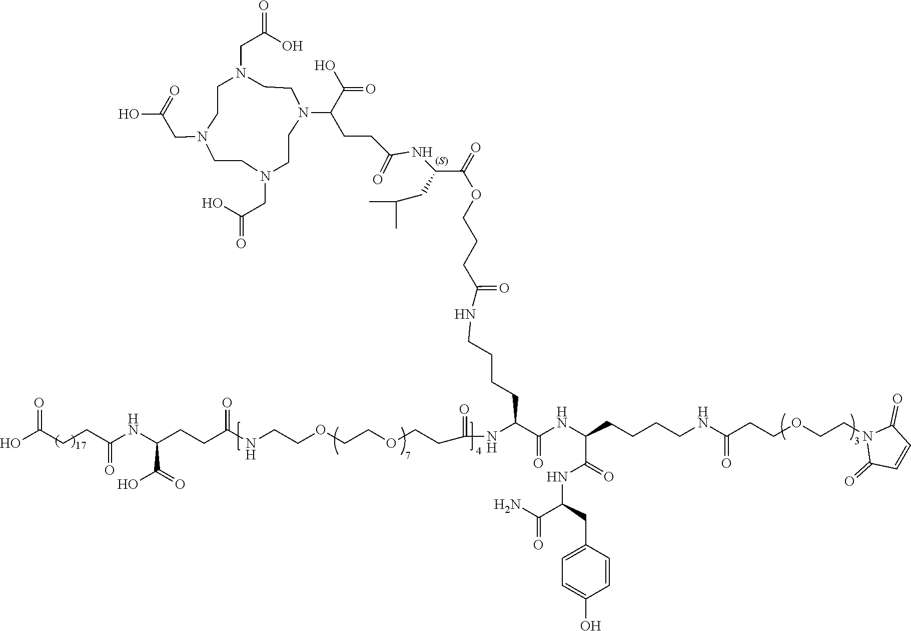 |
| | HO-C20-gGlu-PEG8-PEG8-PEG8-PEG8-Lys(4hBA-Leu-DOTAGA)-Lys(PEG3-MAL)-Tyr-NH2 |
| L081 | 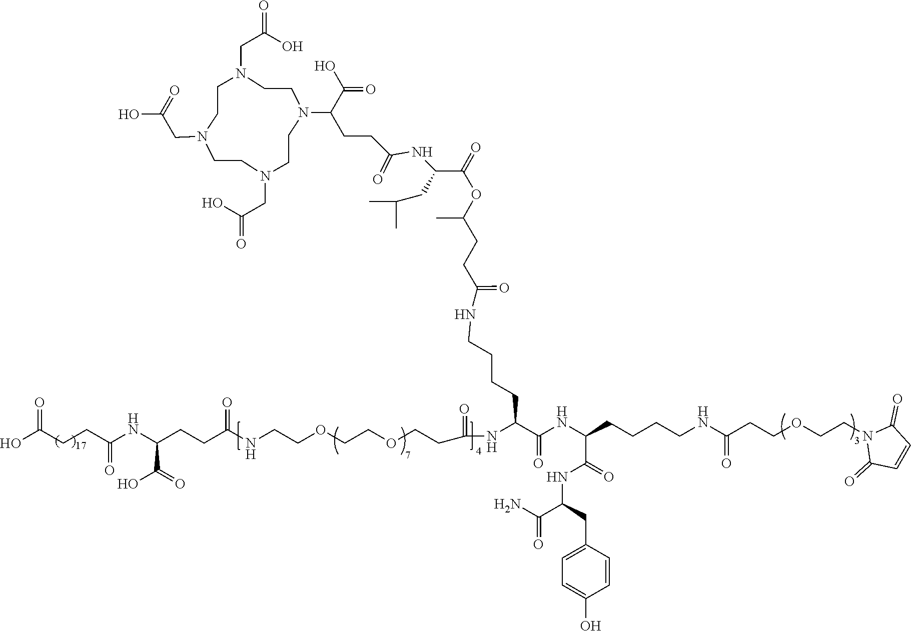 |
| | HO-C20-gGlu-PEG8-PEG8-PEG8-PEG8-Lys(4hPA-Leu-DOTAGA)-Lys(PEG3-MAL)-Tyr-NH2 |
| L082 | 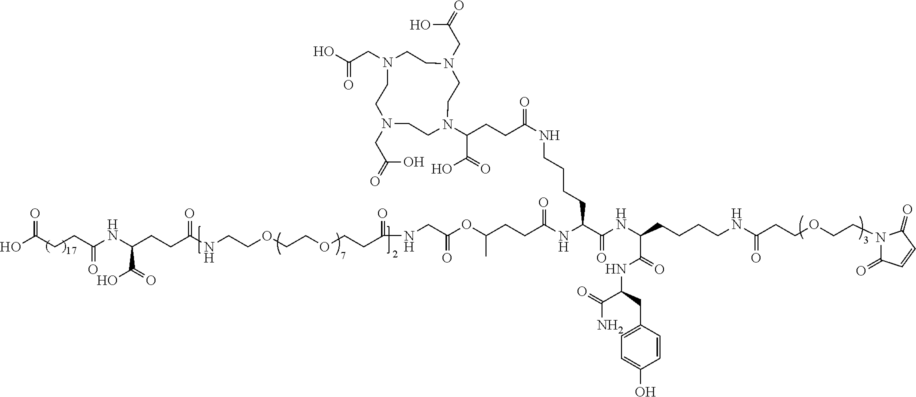 |
| | HO-C20-gGlu-PEG8-PEG8-PEG8-PEG8-Gly-4hPA-Lys(DOTAGA)-Lys(PEG3-MAL)-Tyr-NH2 |
| L083 | 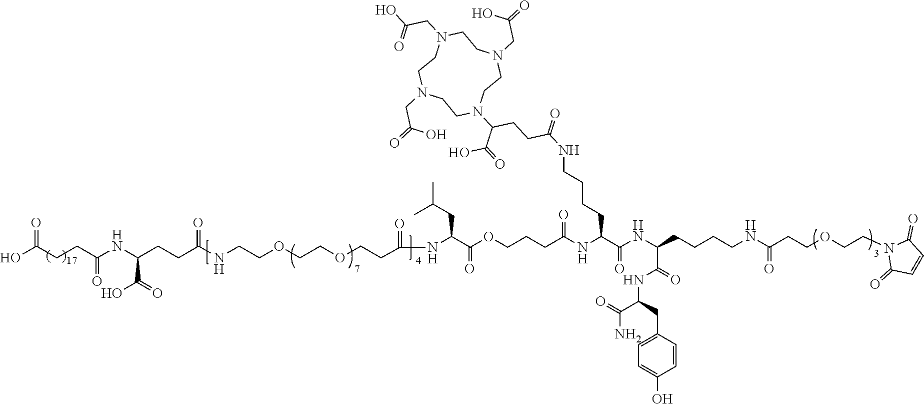 |
| | HO-C20-gGlu-PEG8-PEG8-PEG8-PEG8-Leu-4hBA-Lys(DOTAGA)-Lys(PEG3-MAL)-Tyr-NH2 |
| L084 | 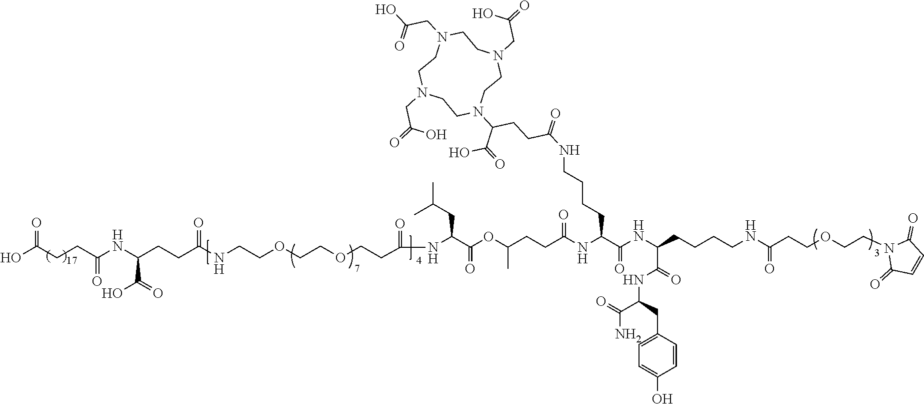 |
| | HO-C20-gGlu-PEG8-PEG8-PEG8-PEG8-Leu-4hPA-Lys(DOTAGA)-Lys(PEG3-MAL)-Tyr-NH2 |
| L085 | 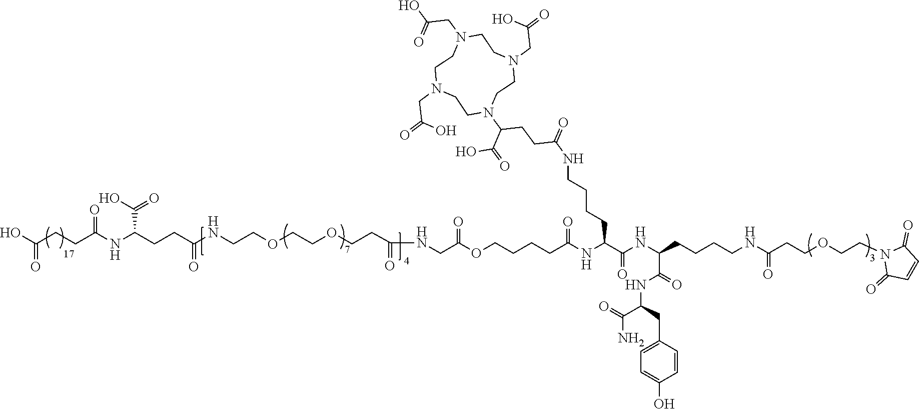 |
| | HO-C20-gGlu-PEG8-PEG8-PEG8-PEG8-Gly-5hPA-Lys(DOTAGA)-Lys(PEG3-MAL)-Tyr-NH2 |
| L086 | 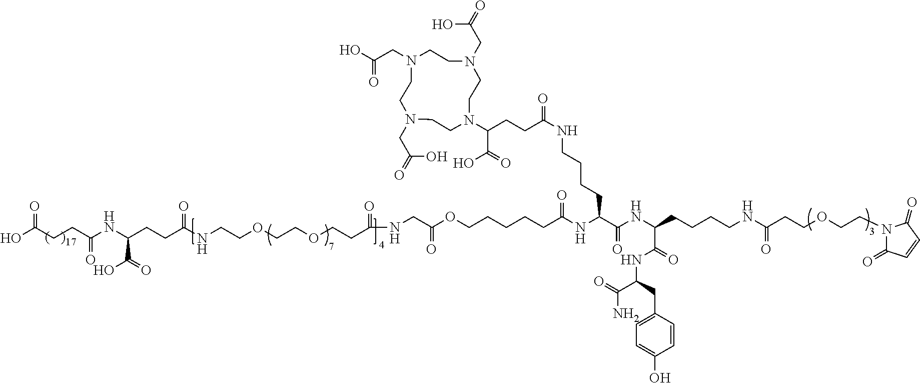 |
| | HO-C20-gGlu-PEG8-PEG8-PEG8-PEG8-Gly-6hHA-Lys(DOTAGA)-Lys(PEG3-MAL)-Tyr-NH2 |
| L087 | 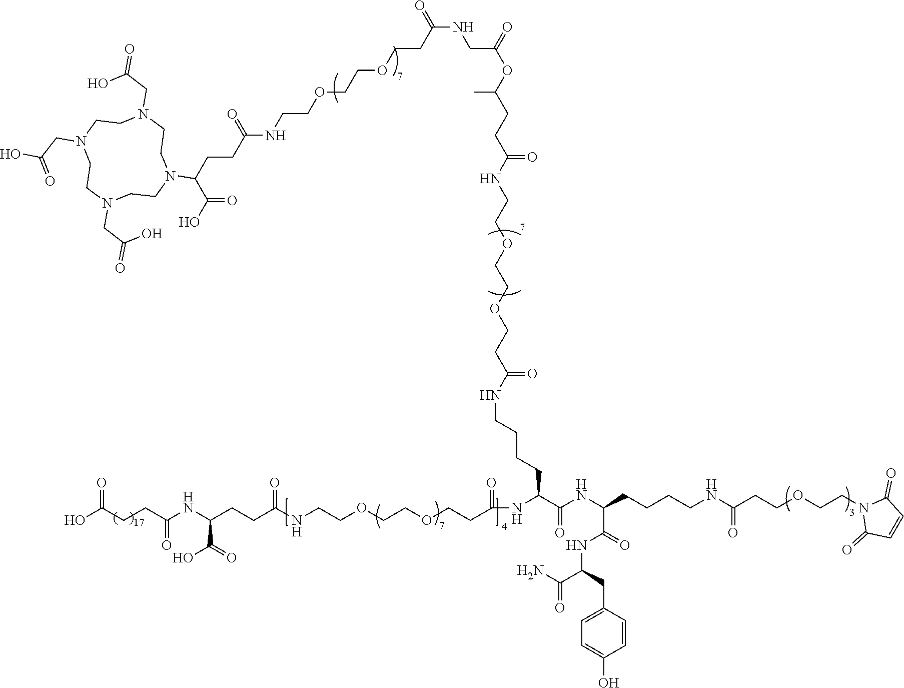 |
| | HO-C20-gGlu-PEG8-PEG8-PEG8-PEG8-Lys(PEG8-4hPA-Gly-PEG8-DOTAGA)-Lys(PEG3-MAL)-Tyr-NH2 |
| L088 | 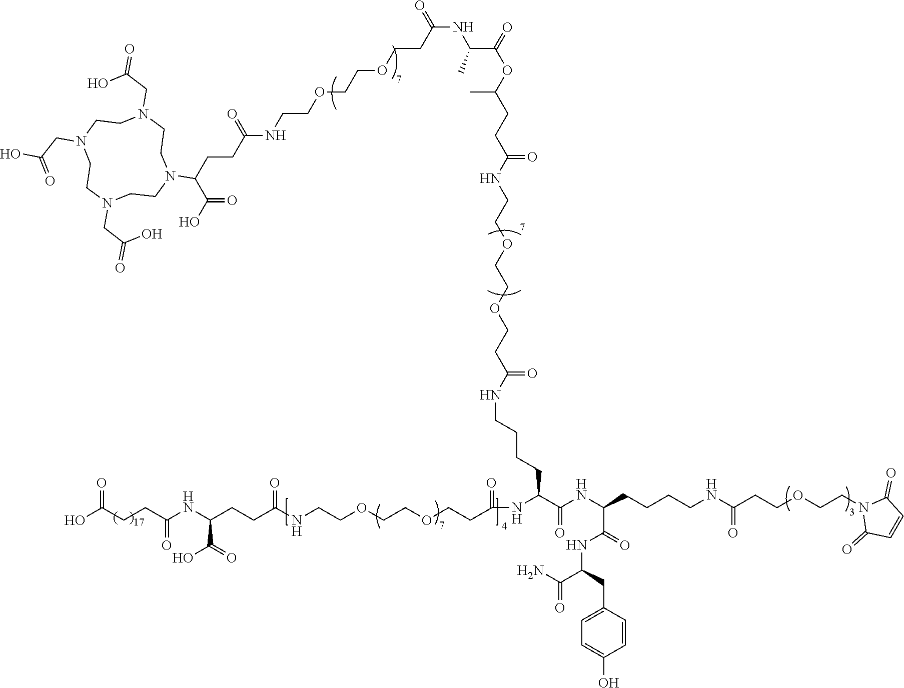 |
| | HO-C20-gGlu-PEG8-PEG8-PEG8-PEG8-Lys(PEG8-4hPA-Ala-PEG8-DOTAGA)-Lys(PEG3-MAL)-Tyr-NH2 |
| L089 | 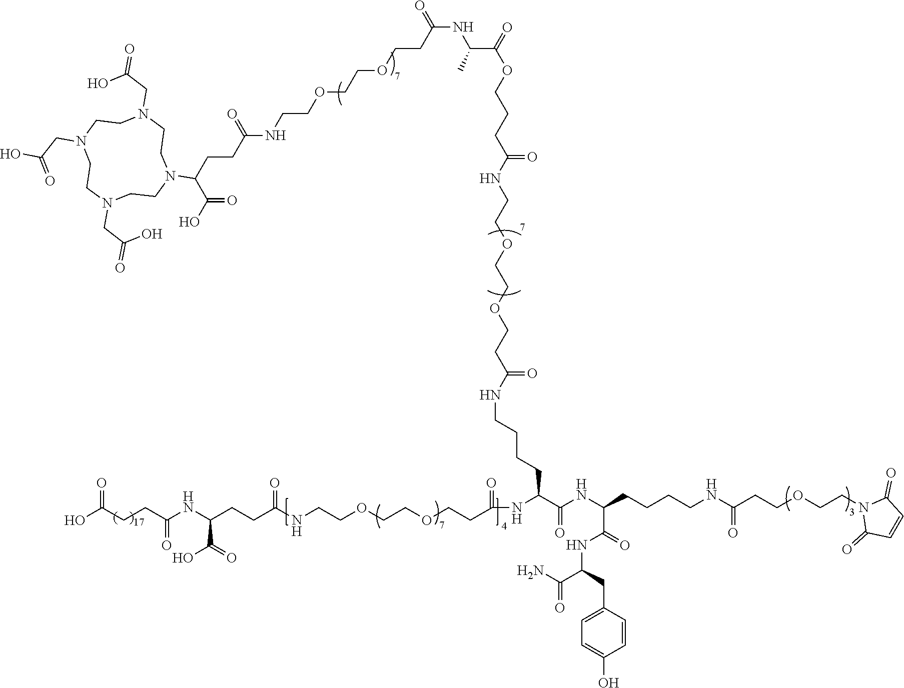 |
| | HO-C20-gGlu-PEG8-PEG8-PEG8-PEG8-Lys(PEG8-4hBA-Ala-PEG8-DOTAGA)-Lys(PEG3-MAL)-Tyr-NH2 |
| L090 | 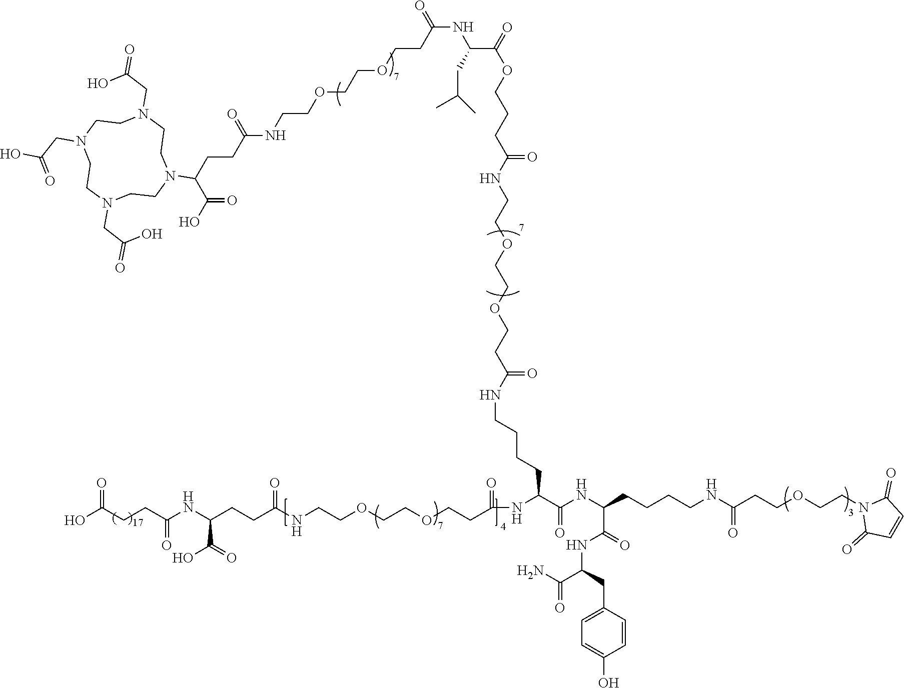 |
| | HO-C20-gGlu-PEG8-PEG8-PEG8-PEG8-Lys(PEG8-4hBA-Leu-PEG8-DOTAGA)-Lys(PEG3-MAL)-Tyr-NH2 |
| L091 | 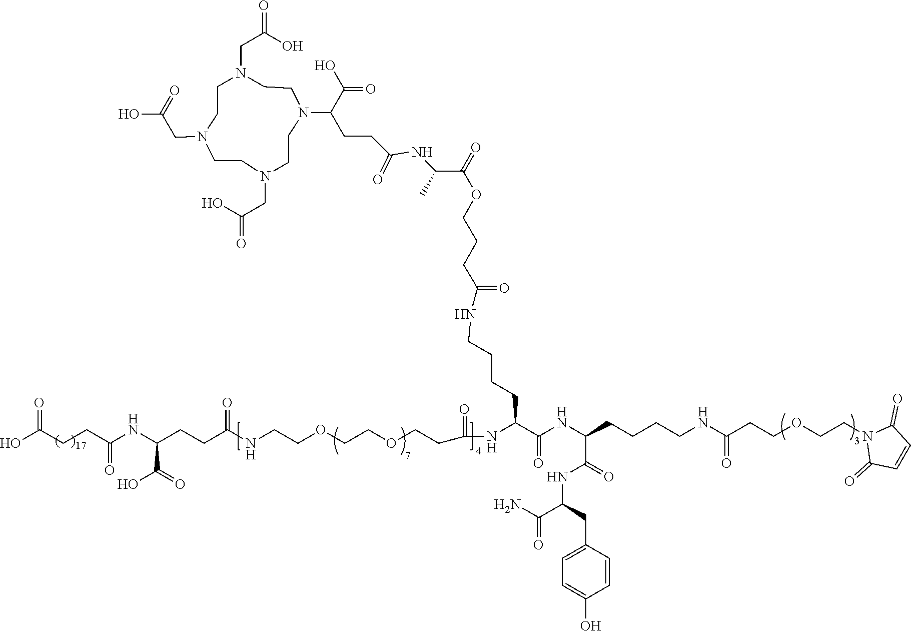 |
| | HO-C20-gGlu-PEG8-PEG8-PEG8-PEG8-Lys(4hBA-Ala-DOTAGA)-Lys(PEG3-MAL)-Tyr-NH2 |
| L092 | 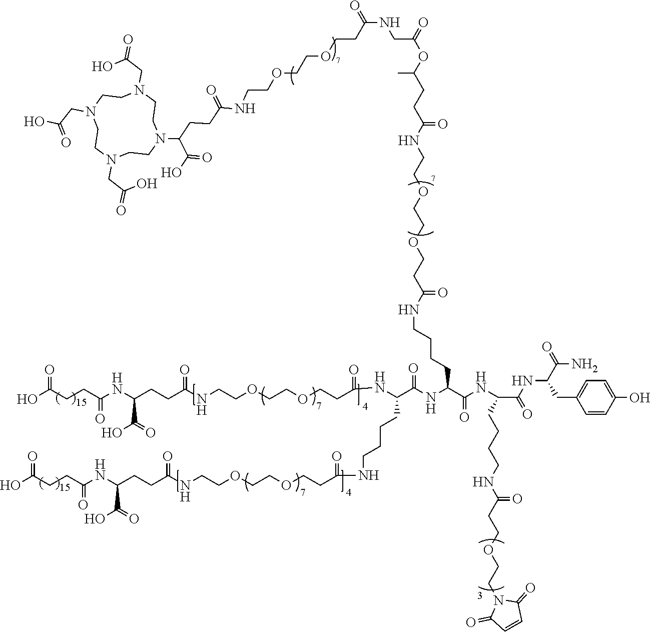 |
| | [HO-C18-gGlu-PEG8-PEG8-PEG8-PEG8]2-Lys-Lys(PEG8-4hPA-Gly-PEG8-DOTAGA)-Lys(PEG3-MAL)-Tyr-NH2 |
| L093 | 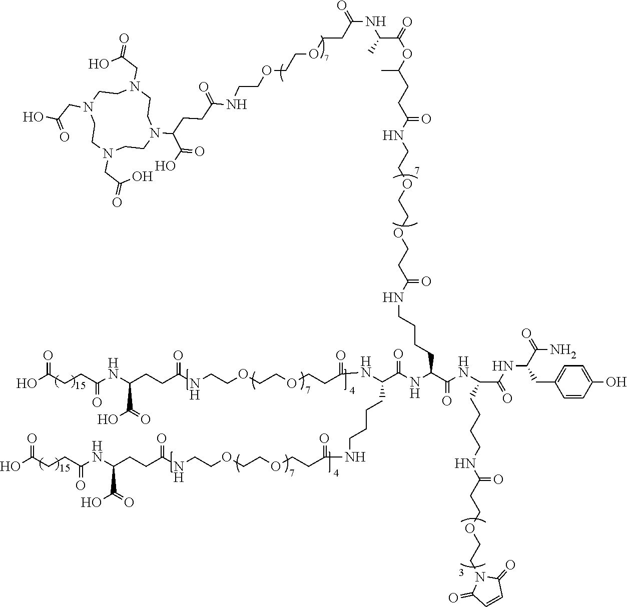 |
| | [HO-C18-gGlu-PEG8-PEG8-PEG8-PEG8]2-Lys-Lys(PEG8-4hPA-Ala-PEG8-DOTAGA)-Lys(PEG3-MAL)-Tyr-NH2 |
| L094 | 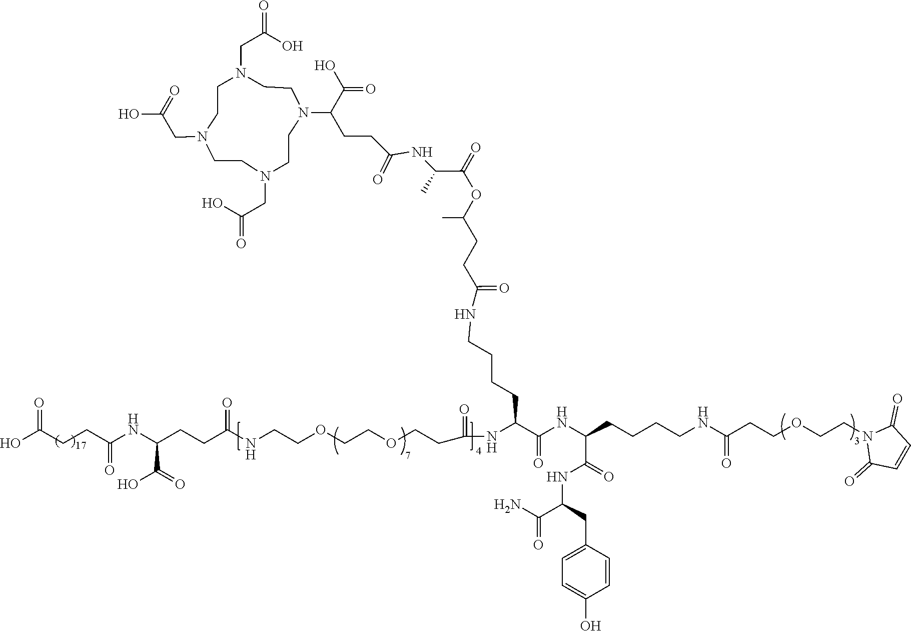 |
| | HO-C20-gGlu-PEG8-PEG8-PEG8-PEG8-Lys(4hPA-Ala-DOTAGA)-Lys(PEG3-MAL)-Tyr-NH2 |
| L095 | 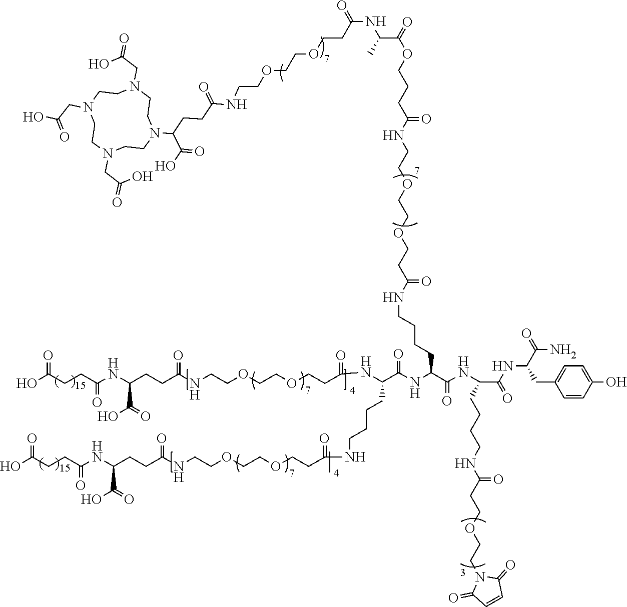 |
| | [HO-C18-gGlu-PEG8-PEG8-PEG8-PEG8]2-Lys-Lys(PEG8-4hBA-Ala-PEG8-DOTAGA)-Lys(PEG3-MAL)-Tyr-NH2 |
| L096 | 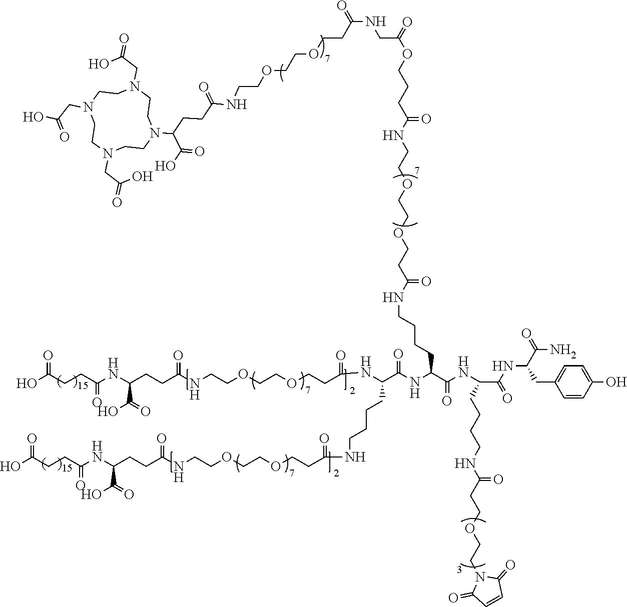 |
| | [HO-C18-gGlu-PEG8-PEG8]2-Lys-Lys(PEG8-ESL1-PEG8-DOTAGA)-Lys(PEG3-MAL)-Tyr-NH2 |
| L097 | 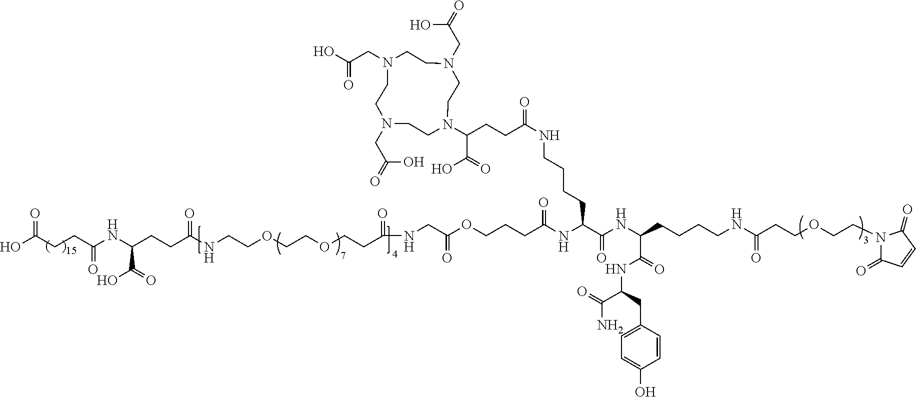 |
| | HO-C18-gGlu-PEG8-PEG8-PEG8-PEG8-ESL1-Lys(DOTAGA)-Lys(PEG3-MAL)-Tyr-NH2 |
| L098 | 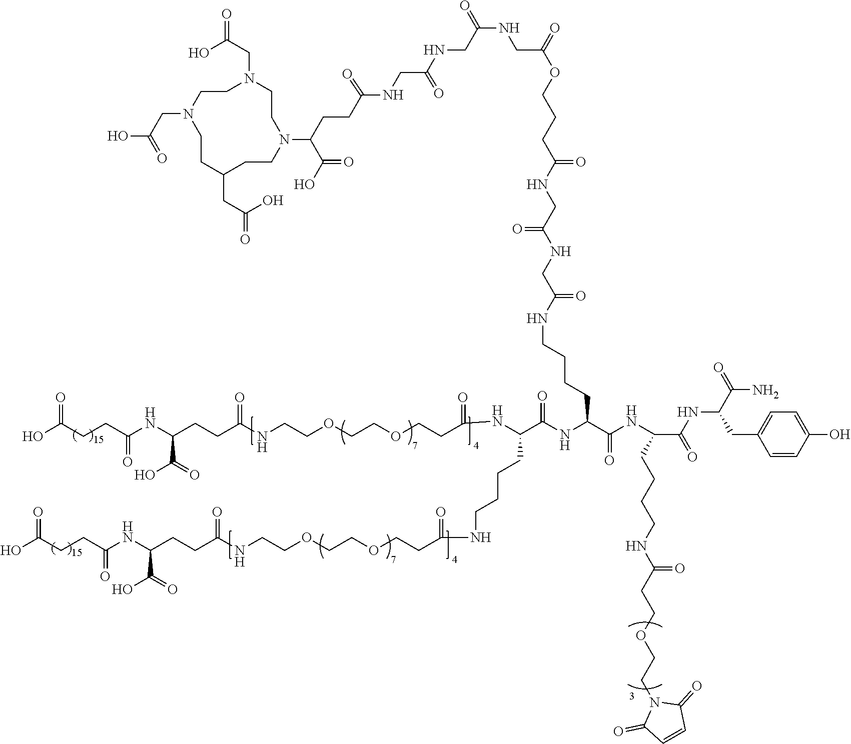 |
| | [HO-C18-gGlu-PEG8-PEG8-PEG8-PEG8]2-Lys-Lys(Gly-Gly-ESL1-Gly-Gly-DOTAGA)-Lys(PEG3-MAL)-Tyr-NH2 |
| L099 | 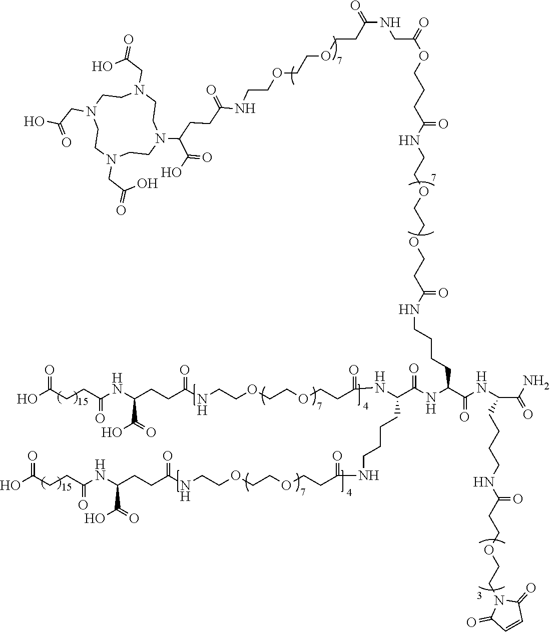 |
| | [HO-C18-gGlu-PEG8-PEG8-PEG8-PEG8-]2-Lys-Lys(PEG8-ESL1-PEG8-DOTAGA)-Lys(PEG3-MAL)-Tyr-NH2 |
| L100 | 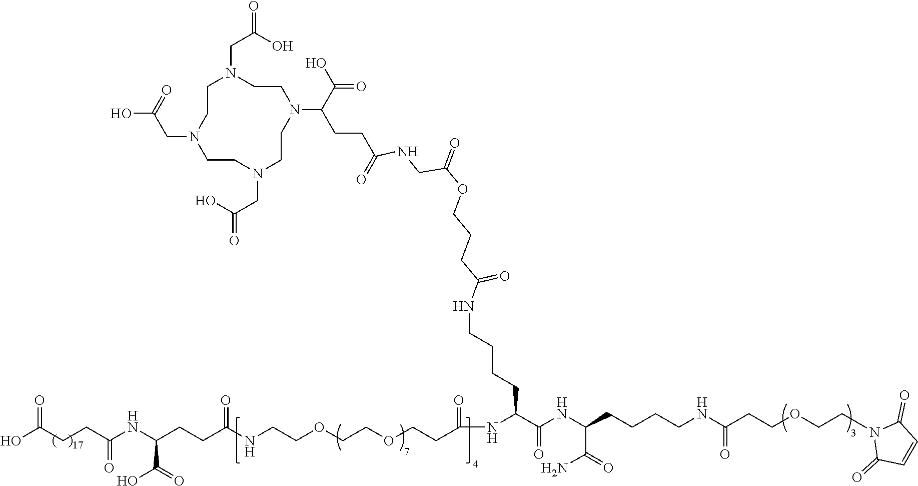 |
| | HO-C20-gGlu-PEG8-PEG8-PEG8-PEG8-Lys(ESL1-DOTAGA)-Lys(PEG3-MAL)-Tyr-NH2 |
| L101 | 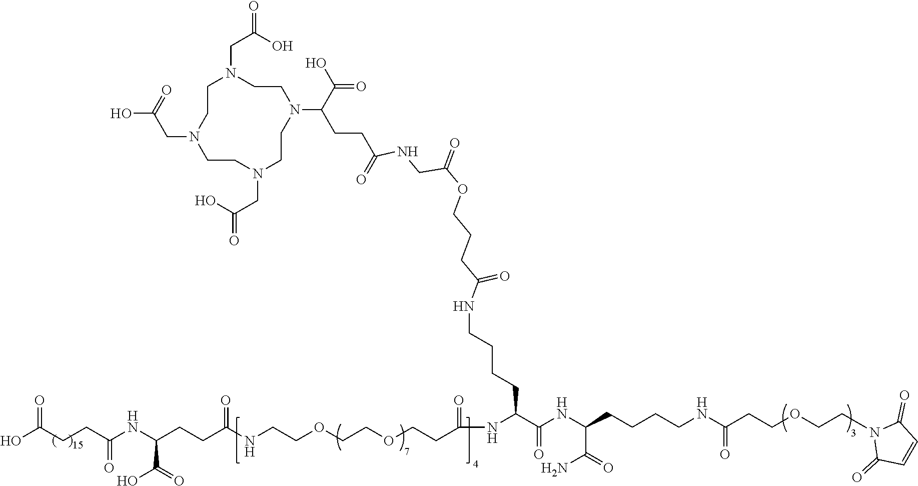 |
| | HO-C18-gGlu-PEG8-PEG8-PEG8-PEG8-Lys(ESL1-DOTAGA)-Lys(PEG3-MAL)-Tyr-NH2 |

### Conjugate

In another aspect, provided herein is a conjugate comprising the compound as described herein and a target binding moiety specifically binds to a target expressed on a cell. In some embodiments, the target is a tumor antigen expressed on a tumor cell. In some embodiments, provided herein is a conjugate comprising the compound as described herein and a tumor targeting moiety specifically binds to a tumor antigen. In some embodiments of the conjugate of the present application, the compound is conjugated with the targeting binding moiety via L^{Z(i)} of the compound.

In some embodiments, the conjugate of the present application comprises a structure of formula (I):

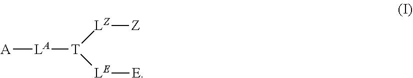

wherein T is a branching group which is at least trivalent; Z is a target binding moiety specifically binding to target expressed on a cell and has a molecular size of less than 66 kDa, wherein Z is conjugated to T via a linker L^{Z}; E is an effector moiety, wherein E is conjugated to T via a linker L^{E}; and A is a potency enhancing moiety, wherein A is conjugated to T via a linker L^{A}, wherein A, L^{A}, E, L^{E} and T are as defined in Formula (I-X).

In some embodiments, the potency enhanced by A includes but is not limited to improved binding affmity against the target that Z specifically binds to, extended in vivo half-life, increased tumor uptake, and/or enhanced in vivo activity, as compared to the conjugate without A.

In some embodiments, Z of the conjugate specifically binds to a tumor antigen expressed on a tumor cell, optionally a cancer cell. In some embodiments, Z comprises an antibody or antibody mimetic. In some embodiments, the antibody or antibody mimetic is selected from the group consisting of a single domain antibody (sdAb), a fragment antigen-binding region (Fab), a single-chain variable fragment (scFv), an affibody, a DARPin (an acronym for designed ankyrin repeat proteins), an Anticalin, an Avimer (avidity multimer), and a Versabody. In some embodiments, Z comprises a single domain antibody (sdAb). In some embodiments, Z comprises an affibody.

In some embodiments, Z of the conjugate described herein has a molecular size of less than about 66 kDa. In some embodiments, Z of the conjugate described herein has a molecular size of less than about 60 kDa. In some embodiments, Z of the conjugate described herein has a molecular size of less than about 50 kDa. In some embodiments, Z of the conjugate described herein has a molecular size of less than about 40 kDa. In some embodiments, Z of the conjugate described herein has a molecular size of less than about 30 kDa. In some embodiments, Z of the conjugate described herein has a molecular size of less than about 20 kDa. In some embodiments, Z of the conjugate described herein has a molecular size of less than about 10 kDa. In some embodiments, Z of the conjugate described herein has a molecular size of less than about 5 kDa. In some embodiments, Z of the conjugate described herein has a molecular size of about 5 kDa to about 66 kDa, about 10 kDa to about 50 kDa about 20 kDa to about 50Kda.

In some embodiments, Z of the conjugate of the present application specifically binds to a tumor antigen selected from the group consisting of Carbonic Anhydrase IX (CAIX), B7 Homolog 3 (B7-H3), Guanylyl cyclase C (GCC), prostate specific membrane antigen (PSMA), fibroblast activation protein alpha (FAP-alpha, FAP), Epidermal Growth Factor Receptor (EGFR), Human Epidermal Growth Factor Receptor-2 (HER-2), Receptor tyrosine-protein kinase erbB-3 (HER-3), Platelet-derived growth factor receptor beta (PDGF-Rβ), Programmed death-ligand 1 (PD-L1), Tumour Necrosis Factor alpha (TNF-α), Vascular endothelial growth factor receptor 2 (VEGFR-2), Vascular Endothelial Growth Factor (VGFR), Vascular Endothelial Growth Factor Receptor (VEGFR), Claudin 18.2, Glucagon-like Peptide-1 Receptor (GLP-1R), Glucose-dependent Insulinotropic Polypeptide Receptor (GIP-R), Cholecystokinin-2 Receptor (CCK2R), Gastrin Releasing Peptide Receptor (GRPR), Somatostatin Receptor 2 (SSTR2), Neurotensin Receptor 1 (NTR1), Neuropeptide Y Receptor Type 1 (Y1R), Nectin-4, Epithelial Cell Adhesion Molecule (EpCAM), and Insulin-like Growth Factor-1 (IGF-1) Receptor. In some embodiments, Z of the conjugate of the present application specifically binds to Claudin 18.2. In some embodiments, Z of the conjugate of the present application specifically binds to GCC. In some embodiments, Z of the conjugate of the present application specifically binds to FAP. In some embodiments, Z of the conjugate of the present application specifically binds to B7-H3.

In some embodiments, Z of the conjugate comprises a sdAb specifically binding to a tumor antigen selected from the group consisting of Guanylyl cyclase C (GCC), prostate specific membrane antigen (PSMA), fibroblast activation protein alpha (FAP-alpha, FAP), Epidermal Growth Factor Receptor (EGFR), Human Epidermal Growth Factor Receptor-2 (HER-2), Vascular Endothelial Growth Factor (VGFR), Vascular Endothelial Growth Factor Receptor (VEGFR), Claudin 18.2, Glucagon-like Peptide-1 Receptor (GLP-1R), Glucose-dependent Insulinotropic Polypeptide Receptor (GIP-R), Cholecystokinin-2 Receptor (CCK2R), Gastrin Releasing Peptide Receptor (GRPR), Somatostatin Receptor 2 (SSTR2), Neurotensin Receptor 1 (NTR1), Neuropeptide Y Receptor Type 1 (Y1R), Nectin-4, Epithelial Cell Adhesion Molecule (EpCAM), or Insulin-like Growth Factor-1 (IGF-1) Receptor. In some embodiments, Z of the conjugate comprises a sdAb specifically binding to Claudin 18.2. In some embodiments, Z of the conjugate comprises a sdAb specifically binding to GCC. In some embodiments, Z of the conjugate comprises a sdAb specifically binding to FAP.

In some embodiments, Z of the conjugate comprises an affibody specifically binding to a tumor antigen selected from the group consisting of Carbonic Anhydrase IX (CAIX), Epidermal growth factor receptor (EGFR), Receptor tyrosine-protein kinase erbB-2 (HER-2), Receptor tyrosine-protein kinase erbB-3 (HER-3), Insulin-like growth factor 1 (IGF-1R), Platelet-derived growth factor receptor beta (PDGF-Rβ), Programmed death-ligand 1 (PD-L1), Tumour Necrosis Factor alpha (TNF-α), Vascular endothelial growth factor receptor 2 (VEGFR-2) or B7 Homolog 3 (B7-H3). In some embodiments, Z of the conjugate comprises an affibody specifically binding to B7-H3.

In some embodiments, the binding affinity of Z to the tumor antigen is in a range of from about 10⁻¹² and about 10⁻⁸ M. In some embodiments, the binding affinity of Z to the tumor antigen is in a range of from about 10⁻¹² to about 10⁻¹¹ M. In some embodiments, the binding affinity of Z to the tumor antigen is in a range of from about 10⁻¹² to about 10⁻¹⁰ M.

In some embodiments, the binding affmity of Z to the tumor antigen is in a range of from about 10⁻¹² to about 10⁻⁹ M. In some embodiments, the binding affinity of Z to the tumor antigen is in a range of from about 10⁻¹¹ to about 10⁻¹⁰ M. In some embodiments, the binding affinity of Z to the tumor antigen is in a range of from about 10⁻¹¹ to about 10⁻⁹ M. In some embodiments, the binding affinity of Z to the tumor antigen is in a range of from about 10⁻¹¹ to about 10⁻⁸ M. In some embodiments, the binding affinity of Z to the tumor antigen is in a range of from about 10⁻¹⁰ to about 10⁻⁹ M. In some embodiments, the binding affinity of Z to the tumor antigen is in a range between about 10⁻¹⁰ and about 10⁻⁸ M. In some embodiments, the binding affinity of Z to the tumor antigen is in a range of from about 10⁻⁹ to about 10⁻⁸ M. In some embodiments, the binding affinity of Z to the tumor antigen is about 10⁻¹², about 10⁻¹¹, about 10⁻¹⁰, about 10⁻⁹ or about 10⁻⁸ M.

In some embodiments, Z comprises one or more Cysteine (Cys) residues and Z is conjugated with L^{Z} via the one or more Cys residues of Z. In some embodiments, Z comprises one or more Lysine (Lys) residues and Z is conjugated with L^{Z} via the one or more Lys residues of Z.

In some embodiments, L^{Z} is covalently conjugated with the Cys amino acid residue of Z and L^{Z} comprises a structure selected from the group consisting of

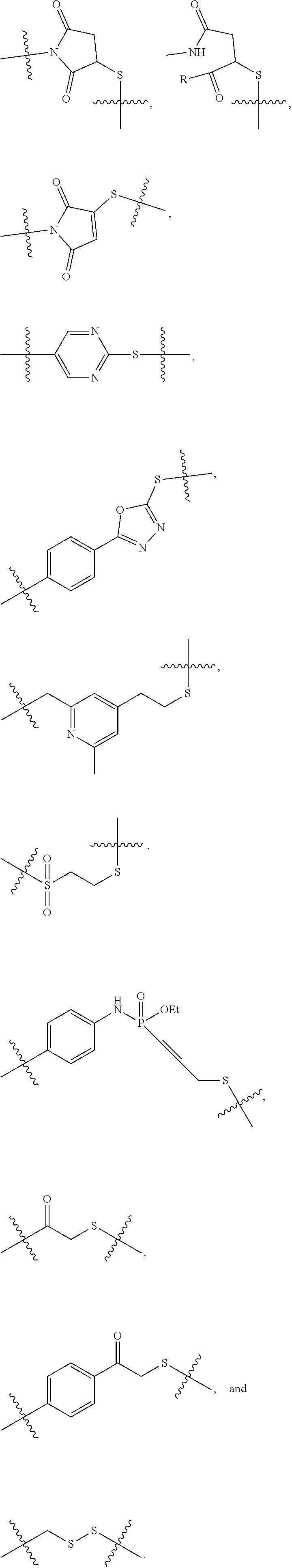

In some embodiments, L^{Z} is covalently conjugated with a Lys amino acid residue of Z and L^{Z} comprises a structure selected from the group consisting of

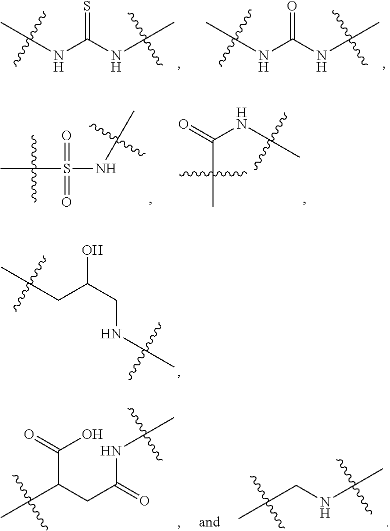

In some embodiments, Z comprises a structure of formula (XI):

B—P (XI),

wherein B is an antibody or antibody mimetic; P is null or P comprises a structure of formula

X—J—Y (XII):

wherein X is null or an amino acid linker, J is a Cys residue, and Y is null or an extension moiety, and wherein X or Y do not contain a residue that is identical to J.

In some embodiments, P is at C-terminus of B. In some embodiments, P is at N-terminus of B. In some embodiments, Z comprises a structure of B-X-J-Y, or Y-J-X-B.

In some embodiments, B is selected from the group consisting of a single domain antibody (sdAb), a fragment antigen-binding region (Fab), a single-chain variable fragment (scFv), an affibody, a DARPin (an acronym for designed ankyrin repeat proteins), an Anticalin, an Avimer (avidity multimer), and a Versabody. In some embodiments, B is a single domain antibody (sdAb). In some embodiments, B is an affibody.

In some embodiments, B binds to a tumor antigen selected from the group consisting of Carbonic Anhydrase IX (CAIX), B7 Homolog 3 (B7-H3), Guanylyl cyclase C (GCC), prostate specific membrane antigen (PSMA), fibroblast activation protein alpha (FAP-alpha, FAP), Epidermal Growth Factor Receptor (EGFR), Human Epidermal Growth Factor Receptor-2 (HER-2), Receptor tyrosine-protein kinase erbB-3 (HER-3), Platelet-derived growth factor receptor beta (PDGF-Rβ), Programmed death-ligand 1 (PD-L1), Tumour Necrosis Factor alpha (TNF-α), Vascular endothelial growth factor receptor 2 (VEGFR-2), Vascular Endothelial Growth Factor (VGFR), Vascular Endothelial Growth Factor Receptor (VEGFR), Claudin 18.2, Glucagon-like Peptide-1 Receptor (GLP-1R), Glucose-dependent Insulinotropic Polypeptide Receptor (GIP-R), Cholecystokinin-2 Receptor (CCK2R), Gastrin Releasing Peptide Receptor (GRPR), Somatostatin Receptor 2 (SSTR2), Neurotensin Receptor 1 (NTR1), Neuropeptide Y Receptor Type 1 (Y1R), Nectin-4, Epithelial Cell Adhesion Molecule (EpCAM), and Insulin-like Growth Factor-1 (IGF-1) Receptor.

In some embodiment, B is a single domain antibody (sdAb) or an affibody that specifically binds to a tumor antigen. In some embodiments, the binding affmity of the sdAb or the affibody to the tumor antigen is in a range of from about 10⁻¹² to about 10⁻⁸ M. In some embodiments, the binding affmity of the sdAb or the affibody to the tumor antigen is in a range of from about 10⁻¹² to about 10⁻¹¹ M. In some embodiments, the binding affinity of the sdAb or the affibody to the tumor antigen is in a range of from about 10⁻¹² to about 10⁻¹⁰ M. In some embodiments, the binding affinity of the sdAb or the affibody to the tumor antigen is in a range of from about 10⁻¹² to about 10⁻⁹ M. In some embodiments, the binding affinity of the sdAb or the affibody to the tumor antigen is in a range of from about 10⁻¹¹ to about 10⁻¹⁰ M. In some embodiments, the binding affinity of the sdAb or the affibody to the tumor antigen is in a range of from about 10⁻¹¹ to about 10⁻⁹ M. In some embodiments, the binding affinity of the sdAb or the affibody to the tumor antigen is in a range of from about 10⁻¹¹ to about 10⁻⁸ M. In some embodiments, the binding affmity of the sdAb or the affibody to the tumor antigen is in a range of from about 10⁻¹⁰ to about 10⁻⁹ M. In some embodiments, the binding affmity of the sdAb or the affibody to the tumor antigen is in a range of from about 10⁻¹⁰ to about 10⁻⁸ M. In some embodiments, the binding affinity of the sdAb or the affibody to the tumor antigen is in a range of from about 10⁻⁹ and about 10⁻⁸ M. In some embodiments, the binding affinity of the sdAb or the affibody to the tumor antigen is about 10⁻¹², about 10⁻¹¹, about 10⁻¹⁰, about 10⁻⁹ or about 10⁻⁸ M.

In some embodiments, the binding affinity of the conjugate to the tumor antigen is in a range of from about 10⁻¹² and about 10⁻⁸ M. In some embodiments, the binding affinity of the conjugate to the tumor antigen is in a range of from about 10⁻¹² to about 10⁻¹¹ M. In some embodiments, the binding affmity of the conjugate to the tumor antigen is in a range of from about 10⁻¹² to about 10⁻¹⁰ M. In some embodiments, the binding affmity of the conjugate to the tumor antigen is in a range of from about 10⁻¹² to about 10⁻⁹ M. In some embodiments, the binding affinity of the conjugate to the tumor antigen is in a range of from about 10⁻¹¹ to about 10⁻¹⁰ M. In some embodiments, the binding affinity of the conjugate to the tumor antigen is in a range of from about 10⁻¹¹ to about 10⁻⁹ M. In some embodiments, the binding affinity of the conjugate to the tumor antigen is in a range of from about 10⁻¹¹ to about 10⁻⁸ M. In some embodiments, the binding affinity of the conjugate to the tumor antigen is in a range of from about 10⁻¹⁰ to about 10⁻⁹ M. In some embodiments, the binding affinity of the conjugate to the tumor antigen is in a range between about 10⁻¹⁰ and about 10⁻⁸ M. In some embodiments, the binding affinity of the conjugate to the tumor antigen is in a range of from about 10⁻⁹ to about 10⁻⁸ M. In some embodiments, the binding affinity of the conjugate to the tumor antigen is about 10⁻¹², about 10⁻¹¹, about 10⁻¹⁰, about 10⁻⁹ or about 10⁻⁸ M.

Claudin 18.2 is a protein encoded by CLDN18 gene in human. Claudin 18.2 is highly expressed in a significant proportion of gastric and pancreatic adenocarcinomas, while normal tissue expression is limited to the epithelium of the stomach.

In some embodiments, B is a single domain antibody specifically binding to Claudin 18.2. In some embodiments B comprises: (1) a CDR1 comprising an amino acid sequence as set forth in SEQ ID NO. 6 or an amino acid sequence with one to three amino acid alterations as compared to SEQ ID NO. 6; (2) a CDR2 comprising an amino acid sequence as set forth in SEQ ID NO. 7 or an amino acid sequence with one to three amino acid alterations as compared to SEQ ID NO. 7; and (3) a CDR3 comprising an amino acid sequence as set forth in SEQ ID NO. 8 or an amino acid sequence with one to three amino acid alterations as compared to SEQ ID NO. 8. In some embodiments, B is a single domain antibody comprising an amino acid sequence having at least 80%, at least 85%, at least 90%, at least 95%, at least 96%, at least 97%, at least 98%, or at least 99% identity with SEQ ID NO. 5. In some embodiments, B is a single domain antibody comprising an amino acid sequence of SEQ ID NO. 5.

Guanylate cyclase 2C, also known as guanylyl cyclase C (GCC), intestinal guanylate cyclase, guanylate cyclase-C receptor, or the heat-stable enterotoxin receptor (hSTAR) is an enzyme that in human is encoded by the GUCY2C gene. It's expressed at the mucosal cells, e.g., at the apical brush border membranes, lining the small intestine, large intestine and rectum, which can regulate intestinal homeostasis and has emerged as a tumor suppressor.

Further, it is universally expressed in advanced metastatic colorectal tumors, as well as other cancer types that arise through intestinal metaplasia, thereby satisfying many characteristics of a compelling target for treating malignancies.

In some embodiments, B is a single domain antibody specifically binding to GCC. In some embodiments B comprises (1) a CDR1 comprising an amino acid sequence as set forth in SEQ ID NO. 11 or an amino acid sequence with one to three amino acid alterations as compared to SEQ ID NO. 11; (2) a CDR2 comprising an amino acid sequence as set forth in SEQ ID NO. 12 or an amino acid sequence with one to three amino acid alterations as compared to SEQ ID NO. 12; and (3) a CDR3 comprising an amino acid sequence as set forth in SEQ ID NO. 13 or an amino acid sequence with one to three amino acid alterations as compared to SEQ ID NO. 13. In some embodiments, B is a single domain antibody comprising an amino acid sequence having at least 80%, at least 85%, at least 90%, at least 95%, at least 96%, at least 97%, at least 98%, or at least 99% identity with SEQ ID NO. 10. In some embodiments, B is a single domain antibody comprising an amino acid sequence of SEQ ID NO. 10.

Fibroblast activation protein alpha (FAP or FAP-alpha) is a 170 kDa single pass type II transmembrane glycoprotein belonging to the dipeptidyl peptidase 4 family. It is highly expressed in cancer-associated fibroblasts (CAFs) and plays an important role in modulating the tumor microenvironment and supports the tumors cells through the release of enzymes, cytokines and growth factor. FAP can provide target specificity to therapeutic agents because in adult humans its expression is restricted to pathologic sites, including cancer, fibrosis, arthritis, wounding, or inflammation. Studies have shown that FAP expression is highly upregulated on reactive CAFs of more than 90% of all primary and metastatic epithelial tumors, while it is generally absent from normal adult tissues. FAP overexpression is associated with poor prognosis and increased risk of metastasis.

In some embodiments, B is a single domain antibody specifically binding to FAP. In some embodiments B comprises (1) a CDR1 comprising an amino acid sequence as set forth in SEQ ID NO. 19 or an amino acid sequence with one to three amino acid alterations as compared to SEQ ID NO. 19; (2) a CDR2 comprising an amino acid sequence as set forth in SEQ ID NO. 20 or an amino acid sequence with one to three amino acid alterations as compared to SEQ ID NO. 20; and (3) a CDR3 comprising an amino acid sequence as set forth in SEQ ID NO. 21 or an amino acid sequence with one to three amino acid alterations as compared to SEQ ID NO. 21. In some embodiments, B is a single domain antibody comprising an amino acid sequence having at least 80%, at least 85%, at least 90%, at least 95%, at least 96%, at least 97%, at least 98%, or at least 99% identity with SEQ ID NO. 18.

In some embodiments, B is a single domain antibody comprising an amino acid sequence of SEQ ID NO. 18.

B7 Homolog 3 (B7-H3, also known as CD276) is a protein belonging to the B7 family of immune regulatory molecules. It is involved in the regulation of immune responses, particularly in the context of tumor immunity. In normal tissues, B7-H3 expression is limited, but in tumors, its overexpression is associated with poor prognosis, tumor aggressiveness, and resistance to conventional therapies.

In some embodiments, B is an affibody specifically binding to B7-H3. In some embodiments, B is an affibody comprising the sequence of SEQ ID NO. 23. In some embodiments, B is an affibody comprising an amino acid sequence having at least 80%, at least 85%, at least 90%, at least 95%, at least 96%, at least 97%, at least 98%, or at least 99% identity with SEQ ID NO. 24. In some embodiments, B is a single domain antibody comprising an amino acid sequence of SEQ ID NO. 24.

In some embodiments, P comprises no more than 20, no more than 19, no more than 18, no more than 17, no more than 16 no more than 15, no more than 14, no more than 13, no more than 12, no more than 11, no more than 10, no more than 8, no more than 7, no more than 6, no more than 5, no more than 4, no more than 3, no more than 2, or no more than 1 amino acid.

In some embodiments, X of P is null or comprises an amino acid sequence selected from the group consisting of(GGS)ₙ, (GSG)ₙ, (GGGS)ₙ or (GGGGS)ₙ, (GGG)ₙ, (EAAAK)ₙ, wherein n is any integer from 0-3. In some embodiments, X comprises an amino acid sequence selected from the group consisting of GGG, GGS, GSG, GGGS or GGGGS.

In some embodiments, Y of P comprises no more than 5, no more than 4, no more than 3, no more than 2, or no more than 1 amino acid.

In some embodiments, Y comprises one or more amino acid residues selected from the group consisting of alanine (Ala, A), arginine (Arg, R), asparagine (Asn, N), aspartic acid (Asp, D), glutamine (Gln, Q), glutamic acid (Glu, E), glutamine (Gln), glycine (Gly, G), histidine (His, H), isoleucine (Ile, I), leucine (Leu, L), methionine (Met, M), phenylalanine (Phe, F), proline (Pro, P), serine (Ser, S), threonine (Thr, T), tryptophan (Trp, W), tyrosine (Tyr, Y), and valine (Val, V).

In some embodiments, Y comprises an amino acid residue with polar uncharged side chain which is adjacent to J. In some embodiments, the amino acid residue with polar uncharged side chain is selected from the group consisting of Ser, Thr, Gln, and Asn. In some embodiments, Y comprises an amino acid residue with positive charged side chain which is adjacent to J. In some embodiments, amino acid residue with positive charged side chain is selected from the group consisting of His, and Arg. In some embodiments, Y comprises an amino acid residue of Pro which is adjacent to J. In some embodiments, Y comprises an amino acid residue selected from the group consisting of Ser, Thr, Gln, Asn, Pro, His, and Arg which is adjacent to J. In some embodiments, Y further comprises one or more additional amino acid residues selected from the group consisting of alanine (Ala, A), arginine (Arg, R), asparagine (Asn, N), aspartic acid (Asp, D), glutamine (Gln, Q), glutamic acid (Glu, E), glutamine (Gln), glycine (Gly, G), histidine (His, H), isoleucine (Ile, I), leucine (Leu, L), methionine (Met, M), proline (Pro, P), serine (Ser, S), threonine (Thr, T), and valine (Val, V).

In some embodiments, Y comprises an amino acid sequence of A, SA, SV, SG, YA, YV, YG. QA, QV, QG, PA, PV, PG, HA, HV, or HG. In some embodiments, Y comprises an amino acid sequence of SA, QA, PA, or HA.

In some embodiments, P comprises an amino acid sequence of GGGC, GGSC, GGGSC, GGGCA, GGGSCA, GGSCA, GGGSCSA, GGGSCYA, GGGSCQA, GGGSCPA, GGGSCHA, GGSCSA, GGSCYA, GGSCQA, GGSCPA, GGSCHA, GGGCSA, GGGCYA, GGGCQA, GGGCPA, GGGCHA, GGGGSCSA, GGGGSCYA, GGGGSCQA, GGGGSCPA, GGGGSCHA, GSGCSA, GSGCYA, GSGCQA, GSGCPA, and GSGCHA.

In some embodiments, P comprises an amino acid sequence as set forth in any one of SEQ ID NO. 1-4, or an amino acid sequence with one to three amino acid alterations as compared to any one of SEQ ID NO. 1-4.

In some embodiments, Z comprises an amino acid sequence having at least 80%, at least 85%, at least 90%, at least 95, at least 96%, at least 97%, at least 98%, at least 99% identity with SEQ ID NO. 9. In some embodiments, Z comprises an amino acid sequence of SEQ ID NO. 9.

In some embodiments, Z comprises an amino acid sequence having at least 80%, at least 85%, at least 90%, at least 95, at least 96%, at least 97%, at least 98%, at least 99% identity with anyone selected from SEQ ID NO. 14-17. In some embodiments, Z comprises an amino acid sequence of anyone selected from SEQ ID NO. 14-17.

In some embodiments, Z comprises an amino acid sequence having at least 80%, at least 85%, at least 90%, at least 95, at least 96%, at least 97%, at least 98%, at least 99% identity with SEQ ID NO. 22. In some embodiments, Z comprises an amino acid sequence of SEQ ID NO. 22.

In some embodiments, Z comprises an amino acid sequence having at least 80%, at least 85%, at least 90%, at least 95, at least 96%, at least 97%, at least 98%, at least 99% identity with SEQ ID NO. 25. In some embodiments, Z comprises an amino acid sequence of SEQ ID NO. 25.

In some embodiments, L^{Z} is covalently conjugated with the thiol group of J. In some embodiments, L^{Z} comprises a structure selected from the group consisting of

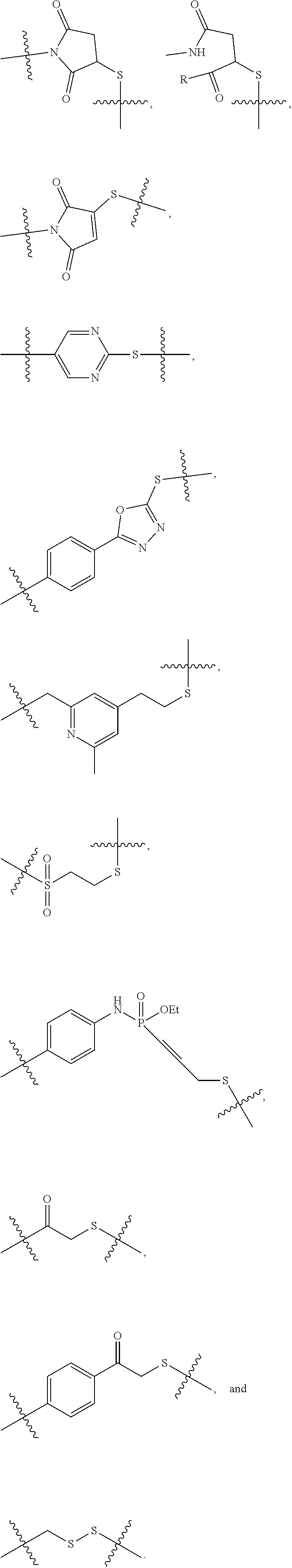

E of the conjugate as described herein comprises a chemotherapeutic agent, a toxin, an immunomodulator, a diagnostic agent, a radionuclide, or a chelating group. In some embodiments, E of the conjugate comprises a chelating group. In some embodiments, E of the conjugate comprises a chelating group which further complexes with one or more radionuclide. In some embodiments, the radionuclides complexed with the chelating group are each independently a radioactive isotope of As, K, Sc, Ti, Cr, Mn, Fe, Co, Ni, Cu, Ga, Ge, Rb, Sr, Y, Zr, Nb, Tc, Rh, Pd, In, Sn, Sb, Zn, Ta, W, Re, Os, Ir, Pt, Au, Hg, Ti, Pb, Bi, Po, Fr, Pm, lanthanide (such as La, Ce, Pr, Nd, Pm, Sm, Eu, Gd, Tb, Dy, Ho, Er, Tm, Yb, Lu), an actinide (such as Ac, Th, U), Mg, Al, Ca, Cd, and Ba. In some embodiments, the lanthanide is Lu, Sm, Ho, or Tb. In some embodiments, the actinide is Ac, Th, or U.

In some embodiments, the one or more radionuclides complexed with the chelating group are each independently selected from the group consisting of ¹⁹⁹Tc, ^{99m}Tc, ¹⁸⁸Re, ¹⁸⁶Re, ¹⁵³Sm, ⁶⁶Ga, ⁶⁷Ga, ⁶⁸Ga, ¹¹¹In, ¹²³In, ⁵⁹Fe, ⁶³Zn, ⁵²Fe, ⁵²Mn, ⁴⁵Ti, ⁶⁰Cu, ⁶¹Cu, ⁶⁷Cu, ⁶⁴Cu, ⁶²Cu, ⁸²Rb, ^{195m}Pt, ^{191m}Pt, ^{193m}Pt, ^{117m}Sn, ⁸⁹Zr, ¹⁷⁷Lu, ¹⁸F, ¹⁸⁸Re, ¹⁸⁶Re, ¹⁵³Sm, ⁶⁶Ho, ⁸⁶Y, ⁸⁷Y, ⁹⁰Y, ⁸⁹Sr ¹⁵³Gd, ¹⁵⁹Gd, ²²⁵Ac, ²¹²Bi, ²¹³Bi, ¹⁹⁸Au, ¹⁹⁹Au, ^{193m}Pt, ¹⁹⁷Pt, ¹⁰³Pd, ¹⁰⁹Pd, ¹⁰⁵Rh, ^{101m}Rh, ^{103m}Rh, ²²³Ra, ²²⁴Ra, ⁹⁷Ru, ²²⁷Th, ²²⁹Th, ¹⁶¹Tb, ¹⁴⁹Tb, ²⁰³Pb, ²¹²Pb, ²⁰¹TI, ¹¹⁹Sb, ^{58m}Co, ⁵⁵Co, ⁵⁷Co ⁴⁷SC, ¹⁴⁹Pm, ¹⁴²Pr, ¹⁶¹Ho, ¹⁶⁶Ho, ¹⁷⁵Yb, and ⁵¹Cr.

In some embodiments, the compound provided herein may be used for imaging, and the one or more radionuclides complexed with the chelating group for use in imaging are selected from the group consisting of ^{99m}Tc, ¹⁸⁸Re, ¹⁸⁶Re, ¹⁵³Sm, ⁶⁶Ga, ⁶⁷Ga, ⁶⁸Ga, ¹¹¹In, ⁵⁹Fe, ⁶³Zn, ⁵²Fe, ⁵²Mn, ⁴⁵Ti, ⁶⁰Cu, ⁶¹Cu, ⁶⁷Cu, ⁶⁴Cu, ⁶²Cu, ⁸²Rb, ¹⁹⁸Au, ¹⁹⁹Au, ^{195m}Pt, ^{191m}Pt, ^{193m}Pt, ^{117m}Sn, ⁸⁹Zr, ¹⁷⁷Lu, ²⁰³Pb, ⁴⁴Sc, ⁵¹Cr, ^{101m}Rh, and ¹⁶⁶Ho.

In some embodiments, the compound provided herein may be used for killing a cell or treating a disease, and the one or more radionuclides for killing a cell or treating a disease are selected from the group consisting of ¹⁸⁸Re, ¹⁸⁶Re, ¹⁵³Sm, ⁶⁶Ho, ⁹⁰Y, ⁸⁹Sr, ¹¹¹In, ¹⁵³Gd, ²²⁵Ac, ²¹²Bi, ²¹³Bi, ⁶⁰Cu, ⁶¹Cu, ⁶⁷Cu, ⁶⁴Cu, ⁶²Cu, ¹⁹⁸Au, ⁹⁹Au, ^{195m}Pt, ^{193m}Pt, ¹⁹⁷Pt, ^{17m}Sn, ¹⁰³Pd, ¹⁰⁵Rh, ^{103m}Rh, ¹⁷⁷Lu, ²²³Ra, ²²⁴Ra, ²²⁷Th, ²²⁹Th, ¹⁴⁹Tb, ¹⁶¹Tb, ²⁰³Pb, ²¹²Pb, ²⁰¹TI, ¹¹⁹Sb, ^{58m}Co ⁴⁷SC, ¹⁴⁹Pm, ¹⁶¹Ho, ¹⁵⁹Gd, ¹⁴²Pr, ¹⁶⁶Ho, and ¹⁷⁵Yb. In some embodiments, the one or more radionuclides for use in therapy are selected from the group consisting of ¹⁷⁷Lu, ²¹²Pb, and ²²⁵Ac. In some embodiments, the radionuclides for killing a cell or treating a disease is ¹⁷⁷Lu or ²²⁵Ac. In some embodiments, the one or more radionuclides for killing a cell or treating a disease is 177Lu. In some embodiments, the one or more radionuclides for killing a cell or treating a disease is ²²⁵Ac.

In some embodiments, E is ¹⁷⁷Lu-DOTA-, ¹⁷⁷Lu-DOTAGA-, ²²⁵Ac-DOTA-, or ²²⁵Ac-DOTAGA-.

In another aspect, the present application relates to a conjugate that target cell surface receptors. The inventors have shown the radionuclide complexes described herein have significantly enhanced the uptake of radionuclide in the targeting tumor while maintaining the off-target normal organ accumulation to a minimum. The normal organs include but are not limited to kidney, blood and muscle.

Although the full-length antibody can effectively deliver radioisotopes to the tumor tissue, and it has been highly successful in delivering non-radioactive cytotoxic drugs (ADCs), its prolonged circulation half-life, however, has been repeatedly found to cause serious safety concerns for RDC therapy application. The discrepancy on the safety profile of these two modalities is mostly due to the fact that an intact full-length antibody-radionuclide conjugate is still radioactive (toxic) while an intact ADC has minimal toxicity.

Consequently, various full-length antibody engineering efforts have been made to address such limitations including FcRn binding motif mutation to weaken the interaction of the antibody to FcRn therefore shorten its circulation time. In addition, direct modifications of antibody with chemical linkers that may accelerate its systemic clearance have also been reported. However, the inherent large size of a full-length antibody (~150 K Da), far beyond renal clearance cut-off of ~60 K Da, has significantly limited the outcome of these approaches.

Concerning the prolonged circulation half-life of a full-length antibody, the antibody fragments that can largely retain the high affinity and specificity profiles of a full-length antibody while with significantly smaller molecular size have attracted great attention and been explored as the targeting motif of RDC drugs. Although it has been demonstrated that these antibody fragments, such as sdAb, Darpin and others, can be cleared significantly faster than a full length antibody from the blood circulation, however its tumor uptake is typically very low and off-target kidney accumulation is extremely high, preventing the development of them as a therapeutic RDC drug for cancer treatment.

To overcome the above-mentioned limitations associated with utilizing antibody fragments to construct a RDC vector, the present inventors describe methods involving the introduction of an albumin binding motif to the antibody fragments. The present inventors have shown that such modification can moderately increase the blood circulation half-life of the antibody fragments and provides significantly higher tumor uptake, and simultaneously limits the renal clearance pathway of the intact radioligand therefore minimize off-target kidney accumulation. To further minimize the amount of radionuclide exposure to the normal organs including blood and to purposely degrade the radioligand to metabolites that are less likely accumulate in non-target receptor expressing cells, the present inventors have introduced a cleavable linker to a radioligand conjugate. Contrary to the drug conjugate concept in the art, the cleavage process of the radioligand conjugates of the application is purposely designed to take place preferably in plasma and throughout the body, rather than at the targeted tumor site. Such cleavage processes do not rely on the specific tumor-enriched enzymes or the unique chemical conditions within tumor microenvironment. Accordingly, such design avoids the impact of heterogeneity nature of the tumor that may considerably impact the anticipated cleavage kinetics, and ensures minimal inter-patient variability of drug (radionuclide) distribution profile in the body.

The present inventors have found that the cleavable moieties such as ester moieties not only accelerate the systemic clearance rate but also positively influence the biodistribution profile of the RDC radioligands. The cleavable RDC radioligand conjugates possess favorable tumor uptake and normal organ accumulation profile that can potentially benefits their therapeutic efficacy and safety window. It was also found that the biodistribution profile can also be significantly impacted by the nature of the potency enhancing group such as albumin binding moiety.

Accordingly, the present application includes a conjugate comprising one or more potency enhancing groups, one or more target binding groups such as one or more antibodies (for example, sdAbs) and/or antibody mimetics (for example, affibody), one or more chelating groups and at least one branching group that is at least trivalent, wherein the branching group that is at least trivalent is connected to at least one target binding group such as an antibody (for example, an sdAb) or antibody mimetic (for example, affibody) directly or through a first non-cleavable linker, to at least one potency enhancing group directly, through a second non-cleavable linker or through a first cleavable linker, and to at least one chelating group directly, through a third non-cleavable linker or through a second cleavable linker, provided the branching group that is at least trivalent is connected to the at least one potency enhancing group through the first cleavable linker, or the branching group that is at least trivalent is connected to the at least one chelating group through the second cleavable linker.

In some embodiments, the potency enhancing group comprises a plasma protein binding group and/or a polyethylene glycol chain. In some embodiments, the potency enhancing groups comprises an albumin binding group.

In some embodiments, the branching group that is at least trivalent is connected to at least one target binding group such as an antibody (for example, sdAb) or antibody mimetic (for example, affibody) directly or through a first non-cleavable linker, to at least one potency enhancing group through a first cleavable linker, and to at least one chelating group through a second non-cleavable linker.

In some embodiments, the branching group that is at least trivalent is connected to at least one target binding group such as an antibody (for example, sdAb) or antibody mimetic (for example, affibody) directly or through a first non-cleavable linker, to at least one potency enhancing group through a first cleavable linker, and directly to at least one chelating group.

In some embodiments, the branching group that is at least trivalent is connected to at least one target binding group such as an antibody (for example, sdAb) or antibody mimetic (for example, affibody) directly or through a first non-cleavable linker, to at least one potency enhancing group directly or through a second non-cleavable linker, and to at least one chelating group through a first cleavable linker.

In some embodiments, the first, second and third non-cleavable linkers resist degradation and the first, second and third non-cleavable linker are less than 5%, less than 4%, less than 3% or less than 2% degraded in the ex vivo mouse plasma after at least about 4 hours, at least about 8 hours, at least about 12 hours, at least about 16 hours, at least about 32 hours, or at least about 48 hour following administration of the conjugate to mouse plasma.

In some embodiments, the first, second and third non-cleavable linkers resist degradation and the conjugate comprising the group is less than 5%, less than 4%, less than 3% or less than 2% degraded in the mouse plasma after at least about 8 hours, at least about 12 hours, at least about 16 hours, at least about 24 hours, or at least about 32 hour following administration of the conjugate to the mouse plasma.

In some embodiments, the first, second and third non-cleavable linkers resist degradation for at least about 2 to about 8 hours, at least about 4 hours to about 16 hours, at least about 12 hours to about 24 hours, at least about 1 day to about 5 days, or at least about 5 days to about 10 days under physiological conditions.

In some embodiments, the first, second and third non-cleavable linkers each independently comprises one or more non-cleavable moieties. In some embodiments, the first, second and third non-cleavable linkers each independently comprises one or more non-cleavable moieties that resist degradation by one or more of acids, bases, reducing agents, oxidizing agents and enzymes.

In some embodiments, the first, second and/or third non-cleavable linkers each comprise one or more non-cleavable moieties that resist degradation by one or more of acids and bases. Therefore, in some embodiments, the first, second and/or third non-cleavable linker resists degradation in the plasma. In some embodiments, the first, second and/or third non-cleavable linker comprises one or more non-cleavable moieties that resist degradation in the ex vivo plasma at about 37° C. for at least about 2 to about 8 hours, at least about 4 hours to about 16 hours, at least about 12 hours to about 24 hours, at least about 1 day to about 5 days, at least about 5 days to about 10 days under or at least about 24 hours following administration of a conjugate comprising the group to the plasma.

In some embodiments, the first, second and third non-cleavable linkers independently comprise one or more non-cleavable moieties that resist degradation by enzymes.

In some embodiments, the first, second and third non-cleavable linkers comprise one or more non-cleavable moieties that resist degradation that are selected from the group consisting of amine bonds, ether bonds, thioether bonds, amide bonds, urea bonds, thiourea groups, thioamide groups or triazole groups. In some embodiments, the triazole group is prepared using click chemistry.

In some embodiments, the first and second cleavable linkers are degradable in ex vivo mouse plasma and have an ex vivo mouse plasma half-life of about 4 hours to about 360 hours, about 6 hours to about 144 hours, about 12 hours to about 120 hours at about 37° C. in mouse plasma.

In some embodiments, the first and second cleavable linkers are degradable in ex vivo mouse plasma and greater than about 5%, about 6%, about 7%, about 8%, about 9% or about 10% of the cleavable linker is degraded after about 48 to about 96 hours, or about 60 to 96 hours at about 37° C. in mouse plasma.

In some embodiments, the first and second cleavable linkers each comprise one or more cleavable moieties that are degradable by one or more of acids, bases, reducing agents, oxidizing agents and enzymes.

In some embodiments, the first and second cleavable linkers each comprise one or more cleavable moieties that are degradable by enzymes.

In some embodiments, the one or more cleavable moieties are independently selected from the group consisting of an ester group, a disulfide bond, a thioester group, a carbamate group, a carbonate group, a hydrazone bond, an oxime bond such as a ketoxime or aldoxime bond, or enzymatically cleavable peptide sequences.

In some embodiments, the first and second cleavable linkers comprise one or more cleavable moieties that are degradable by one or more of acids and bases. Therefore, in some embodiments, the first and second cleavable linker are degradable in the circulating blood and ex-vivo plasma such as mouse plasma.

Accordingly, in some embodiments, the first and second cleavable linkers each independently comprises at least one cleavable moiety that is cleavable in the circulating blood and ex vivo plasma. Accordingly, in some embodiments, the first and second cleavable linkers each independently comprises one or more cleavable moieties that are cleavable in the circulating blood and ex vivo plasma. In some embodiments, the first and second cleavable linkers independently comprise one to four cleavable moieties that are cleavable in the circulating blood and ex vivo plasma. In some embodiments, the first and second cleavable linkers independently comprise one to three cleavable moieties that are cleavable in the circulating blood and ex vivo plasma. In some embodiments, the first and second cleavable linkers independently comprise one or two cleavable moieties that are cleavable in the circulating blood and ex vivo plasma. In some embodiments, the first and second cleavable linkers independently comprise one cleavable moiety that is cleavable in the circulating blood and ex vivo plasma.

In some embodiments, the first and second cleavable linker each independently comprise at least one cleavable moiety that is cleavable in the ex-vivo mouse plasma and has an ex vivo mouse plasma half-life at about 37° C. of at least about 4 hours to about 360 hours, about 6 hours to about 144 hours, about 12 hours to about 120 hours, about 18 hours to about 108 hours, or about 24 hours to 96 hours following administration of the conjugate to the mouse plasma.

In some embodiments, the conjugate (optionally the first and second cleavable linkers) comprises one to four cleavable moieties. In some embodiments, the conjugate (optionally the first and second cleavable linkers) comprises one to three cleavable moieties.

In some embodiments, the conjugate (optionally the first and second cleavable linkers) comprises three cleavable moieties. In some embodiments, the conjugate comprises two cleavable moieties. In some embodiments, the conjugate (optionally the first and second cleavable linkers) comprises one or two cleavable moieties. In some embodiments, the conjugate (optionally the first and second cleavable linkers) comprises one cleavable moiety.

It would be appreciated by a person skilled in the art that the first, second and third non-cleavable linkers do not comprise a cleavable moiety, while for the cleavable linkers it is an option that they can further comprise non-cleavable moieties. It would be appreciated by a person skilled in the art that the non-cleavable linker group comprises a functional group on each of the termini that reacts with complementary functional groups of the molecules to be linked to form non-cleavable moieties.

It would be further appreciated by a person skilled in the art that the non-cleavable and cleavable linkers join two or more molecular structures together through connections (e.g., functional groups) and that for non-cleavable linkers the connections between the linker and the molecules to be joined are non-cleavable and for cleavable linkers it is an option that the connections between the cleavable linkers and the molecules to be joined can also be cleavable.

Further, it would be appreciated by a person skilled in the art that the branching group that is at least trivalent comprises a functional group on each terminus that reacts with a complementary functional group of each of the at least three molecules to be linked. In some embodiments, the branching group that is at least trivalent comprises a functional group on each termini that reacts with a complementary functional group of the potency enhancing group, the target binding group such as the antibody (for example, the sdAb) or antibody mimetic, and/or the chelating group when bound directly to any one of these groups, or to the first, second and/or third non-cleavable linkers, and/or the first and second cleavable linkers.

It would be further appreciated by a person skilled in the art that the branching group that is at least trivalent comprises a functional group on each termini that connects to the potency enhancing group, the target binding group such as the antibody (for example, the sdAb) or antibody mimetic, and/or the chelating group, or to the first, second and/or third non-cleavable linkers, and/or to the first and second cleavable linkers through connections (e.g., functional groups) and that, when joining to the first, second and/or third non-cleavable linkers or to the target binding group such as the antibody (for example, the sdAb) or antibody mimetic (for example, affibody), the connections between the branching group that is at least trivalent and the first, second and/or third non-cleavable linkers or the target binding group such as the antibody (for example, the sdAb) or antibody mimetic (for example, affibody) to be joined are non-cleavable, and when joining to the first and/or second cleavable linkers, to the potency enhancing groups and/or the target binding groups such as the antibody (for example, the sdAb) or antibody mimetic (for example, affibody), the connections between the branching group that is at least trivalent and the first and/or second cleavable linkers, the potency enhancing groups and/or the target binding groups such as the antibody (for example, the sdAb) or antibody mimetic (for example, affibody) are non-cleavable or cleavable.

In some embodiments, the branching group that is at least trivalent comprises at least a first terminal functionality, a second terminal functionality and a third terminal functionality that connects to the target binding groups such as the antibody (for example, the sdAb) or antibody mimetic (for example, affibody) directly or through the first non-cleavable linker, to the potency enhancing groups directly or through the second non-cleavable linker or through the first cleavable linker and to the chelating groups directly or through the third non-cleavable linker or through the second cleavable linker, respectively.

In some embodiments, the non-cleavable linker groups (e.g., the first non-cleavable linker, the second non-cleavable linker and the third non-cleavable groups) and the cleavable linker groups (e.g., the first cleavable linker and the second cleavable linker groups) optionally comprise functional groups, in addition to the functional groups on each of the termini, that react with complementary functional groups of molecules, in addition to the one target binding group such as the antibody (for example, the sdAb) or antibody mimetic (for example, affibody), the one potency enhancing group or the one chelating group, to be linked. In some embodiments, the molecules in addition to the target binding group such as the antibody (for example, the sdAb) or antibody mimetic (for example, affibody), the potency enhancing group or the chelating group, are other target binding groups such as the antibodies (for example, the sdAbs) or antibody mimetics (for example, affibody), potency enhancing groups and/or chelating groups, and/or other cleavable or non-cleavable linkers.

In some embodiments, the first non-cleavable linker further connects a second target binding group such as an antibody (for example, the sdAb) or antibody mimetic (for example, affibody) to the branching group, the second non-cleavable linker or the first cleavable linker further connects a second potency enhancing group to the branching group and/or the third non-cleavable linker or the second cleavable linker connects a second chelating group to the branching group. In some embodiments, the first non-cleavable linker further connects a second target binding group such as an antibody (for example, the sdAb) or antibody mimetic (for example, affibody) to the branching group. In some embodiments, the second non-cleavable linker or the first cleavable linker further connects a second potency enhancing group to the branching group. In some embodiments, the third non-cleavable linker or the second cleavable linker connects a second chelating group to the branching group.

In some embodiments, the conjugate comprises formula (I):

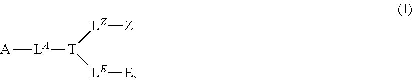

wherein (1) T is a branching group which is at least trivalent, (2) Z is an antibody or antibody mimetic specifically binding to a tumor antigen expressed on a tumor cell, wherein Z is conjugated to T via a linker L^{Z} and has a molecular size of less than 66 kDa, (3) E is an effector moiety, wherein E is conjugated to T via a linker L^{E}, and (4) A is a potency enhancing group, wherein A is conjugated to T via a linker L^{A}. In some embodiments, at least one of L^{E} and L^{A} is cleavable.

In some embodiments of the conjugate of the present application, the potency enhancing group (A) comprises one or more plasma protein binding groups. Any suitable plasma protein binding group known in the art can be used in the conjugate of the present application. In some embodiments, the plasma protein is selected from the group consisting of albumin, alpha-1-acid glycoprotein, fetuin, transferrin, IgG, HDL (high-density lipoprotein) and LDL (low-density lipoprotein). In some embodiments, the plasma protein binding group comprises an albumin binding group or an albumin binder. In some embodiments, A comprises one or more polyethylene glycol chains.

In some embodiments, A comprises one or more albumin binding groups. In some embodiments, the albumin binding group is selected from the group consisting of

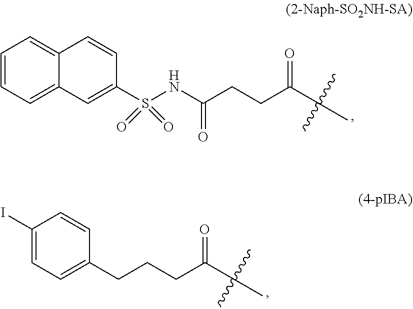 [SMILES: *C(=O)CCC(=O)NS(=O)(=O)c1ccc2ccccc2c1 ; *C(=O)CCCc1ccc(I)cc1]

unsubstituted or substituted C(O)C₁₋₂₆alkyleneCOOH, unsubstituted or substituted C(O)C₁₋₂₆alkenyleneCOOH, unsubstituted or substituted C(O)C₁₋₂₆alkyl, unsubstituted or substituted C(O)C₁₋₂₆alkenyl, and any combination thereof

In some embodiments, the albumin binding group is selected from the group consisting of Naph-SO₂NH-Su, 4-pIBA, unsubstituted or substituted C(O)C₆₋₂₀alkyleneCOOH, unsubstituted or substituted C(O)C₆₋₂₀alkenyleneCOOH, unsubstituted or substituted C(O)C₆₋₂₀alkyl, unsubstituted or substituted C(O)C₆₋₂₀alkenyl, or any combination thereof.

In some embodiments, the albumin binding group is selected from the group consisting of Naph-SO₂NH-Su, 4-pIBA, unsubstituted or substituted C(O)C₁₀₋₂₀alkyleneCOOH and unsubstituted or substituted C(O)C₁₀₋₂₀alkenyleneCOOH, unsubstituted or substituted C(O)C₁₁₋₁₉alkyl, unsubstituted or substituted C(O)C₁₁₋₁₉alkenyl, or any combination thereof.

In some embodiments, the albumin binding group is selected from the group consisting of Naph-SO₂NH-Su, 4-pIBA, unsubstituted or substituted C(O)C₁₋₁₈alkyleneCOOH, unsubstituted or substituted C(O)C₁₄₋₁₉alkyl, or any combination thereof. In some embodiments, the albumin binding group is selected from the group consisting of

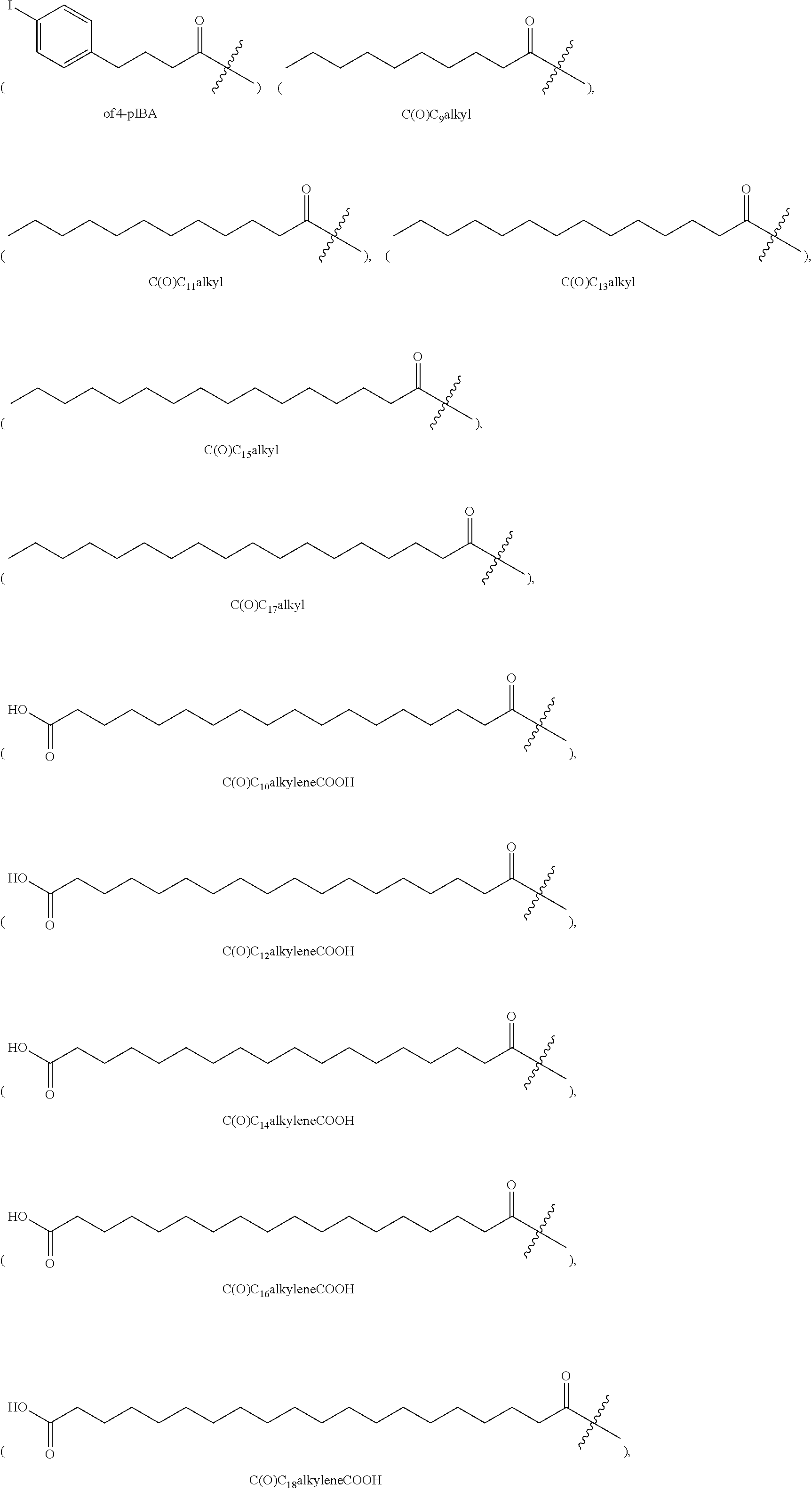

and any combination thereof.

In some embodiments, the albumin binding group is selected from the group consisting of

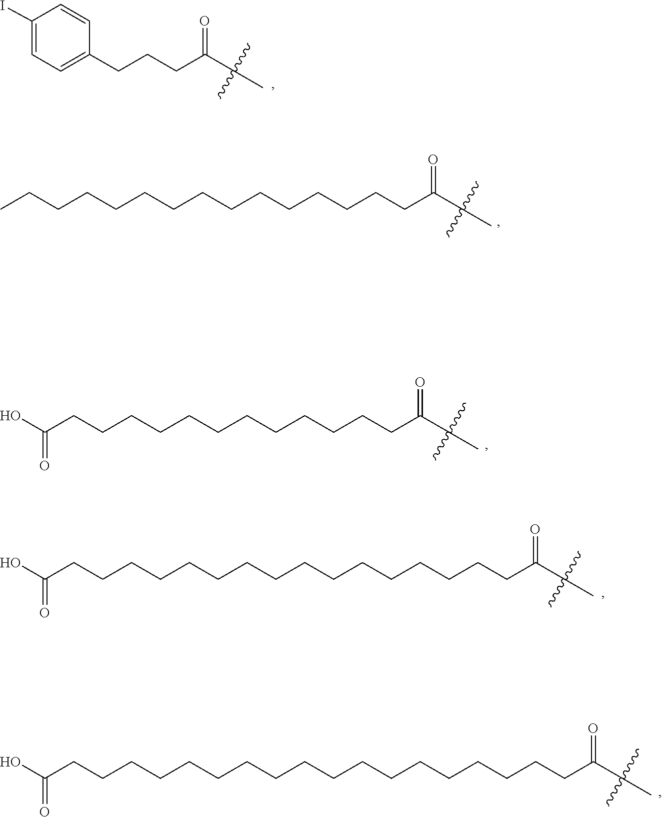 [SMILES: *C(=O)CCCc1ccc(I)cc1 ; *C(=O)CCCCCCCCCCCCCCC ; *C(=O)CCCCCCCCCCCCC(=O)O ; *C(=O)CCCCCCCCCCCCCCCCC(=O)O ; *C(=O)CCCCCCCCCCCCCCCCCCC(=O)O]

and any combination thereof.

In some embodiments, the albumin binding group is selected from the group consisting of unsubstituted or substituted C(O)C₁₂₋₁₈alkyleneCOOH. In some embodiments, the albumin binding group is selected from the group consisting of C(O)C₁₂₋₁₈alkyleneCOOH. In some embodiments, the albumin binding group is selected from the group consisting of

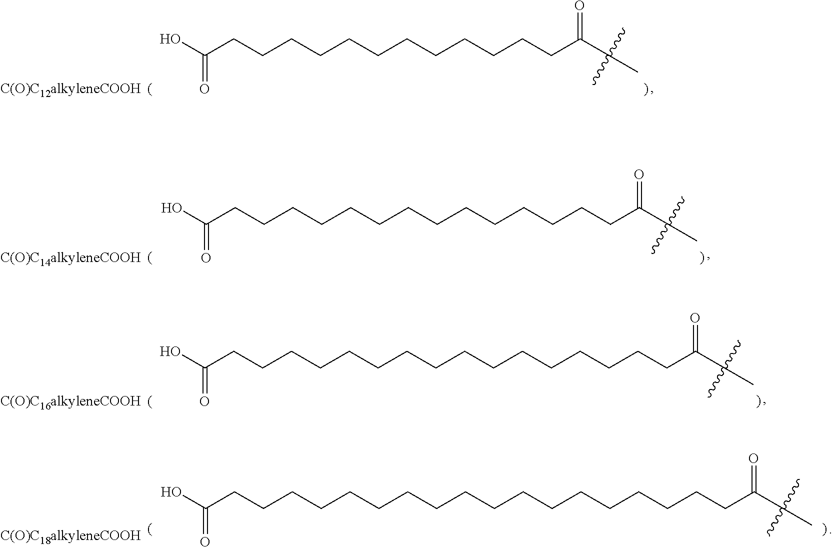 [SMILES: *C(=O)CCCCCCCCCCCCC(=O)O ; *C(=O)CCCCCCCCCCCCCCC(=O)O ; *C(=O)CCCCCCCCCCCCCCCCC(=O)O ; *C(=O)CCCCCCCCCCCCCCCCCCC(=O)O]

and any combination thereof.

In some embodiments, the albumin binding group is selected from the group consisting of C(O)C₁₀alkyleneCOOH, C(O)C₁₂alkyleneCOOH, C(O)C₁₋₄alkyleneCOOH, C(O)C₁₆alkyleneCOOH, C(O)C₁₈alkyleneCOOH, and any combination thereof. In some embodiments, the albumin binding group is C(O)C₁₂alkyleneCOOH. In some embodiments, the albumin binding group is C(O)C₁₆alkyleneCOOH.

In some embodiments, when substituted each C(O)C₆₋₂₀alkyleneCOOH, C(O)C₆₋₂₀alkenyleneCOOH, C(O)C₆₋₁₈alkyl and C(O)C₂₋₂₀alkenyl is substituted with one or more of halo, COOH, CO₂C₁-C₄alkyl, C(O)NH₂, C(O)N(CH₃)₂, C(O)NHCH₃, SO₂CH₃, C₁-C₄alkyl, C₁-C₄fluoralkyl, C₂-C₆alkenyl, C₂-C₆fluoroalkenyl, C₂-C₆alkynyl, C₂-C₆fluoroalkynyl, C₃-C₆cycloalkyl and a 3- to 6-membered heterocyclic ring including 1 to 2 ring members selected from the group consisting of O, S, S(O), SO₂, N, NH and NCH₃.

In some embodiments, the present inventors have found that conjugates of Formula I comprising C(O)C₁₂₋₁₈alkyleneCOOH as the albumin binding group demonstrate a higher tumor to kidney uptake ratio. Therefore, in an exemplary embodiment, the albumin binding group (A) is selected from the group consisting of unsubstituted or substituted C(O)C₁₂₋₁₈alkyleneCOOH. Accordingly, in some embodiments, the conjugate of Formula I is a conjugate of Formula I-A

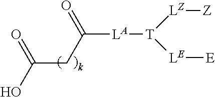

wherein
Z, E, T, L^{A}, L^{E} and L^{Z} are as defined in Formula I, and
k is an integer from 10 to 20.

In some embodiments, at least one of L^{A} and L^{E} is a cleavable linker.

In some embodiments, k is 12 to 18. In some embodiments, k is 14 to 18. In some embodiments, k is 16 to 18. In some embodiments, k is 16 or 18.

In some embodiments, Z is an antibody or antibody mimetic such as a Claudin 18.2 sdAb, a FAP sdAb, a GCC sdAb or a B7-H3 affibody, and the albumin binding group (A) is an unsubstituted or substituted C(O)C₁₀₋₁₈alkyleneCOOH. In some embodiments, Z is an antibody or antibody mimetic such as a Claudin 18.2 sdAb, a FAP sdAb, a GCC sdAb or a B7-H3 affibody, and the albumin binding group (A) is C(O)C₁₀₋₁₈alkyleneCOOH. Accordingly, in some embodiments, when the Z is an antibody or antibody mimetic such as a Claudin 18.2 sdAb, a FAP sdAb, a GCC sdAb or a B7-H3 affibody, and the albumin binding group (A) is an unsubstituted or substituted C(O)C₁₀₋₁₈alkyleneCOOH, the conjugate of Formula I is a conjugate of Formula I-A′

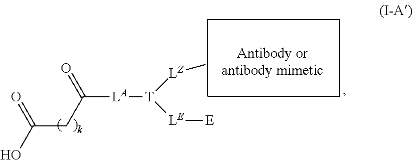

wherein
E, T, L^{A}, L^{E} and L^{Z} are as defined in Formula I, and
k is an integer from 10 to 20.

In some embodiments, at least one of L^{A} and L^{E} is a cleavable linker.

In some embodiments of the conjugate of the present application, T is a branching group that is at least trivalent comprising at least a first terminal functionality, a second terminal functionality and a third terminal functionality, which are the same or different and bind to L^{A} (or alternatively A), L^{Z} (or alternatively Z) and L^{E} (or alternatively E), respectively. In some embodiments, T comprises at least a first terminal functionality, a second terminal functionality and a third terminal functionality which are the same or different and when bonded to L^{A} (or alternatively A), L^{Z} (or alternatively Z) and L^{E} (or alternatively E), respectively, independently form amide group, an amine group, a urea group, a thioether group, a thiourea group or a thioamide group. In some embodiments, when bonded to L^{A} (or alternatively A), and/or L^{E} (or alternatively E), an ester group, a thioester group, a carbonate group, a carbamate group, a disulfide bond, a hydrazone group, or an oxime group such as a ketoxime or aldoxime is further formed. In some embodiments, when bonded to L^{A} (or alternatively A), and/or L^{E} (or alternatively E) an enzymatically cleavable sequence is formed.

In some embodiments, T comprises at least a first terminal functionality, a second terminal functionality and a third terminal functionality which are the same or different and when bonded to L^{A} (or alternatively A), L^{Z} (or alternatively Z) and L^{E} (or alternatively E), respectively independently form an amide group. In some embodiments, when bonded to L^{A} (or alternatively A), and/or L^{E} (or alternatively E), an amide group, an ester group or a disulfide bond group is formed. In some embodiments, when bonded to L^{A} (or alternatively A), and/or L^{E} (or alternatively E), an ester group is formed. In some embodiments, T comprises at least a first terminal functionality, a second terminal functionality and a third terminal functionality which are the same or different and when bonded to L^{A} (or alternatively A), L^{Z} (or alternatively Z) and L^{E} (or alternatively E), respectively independently form an amide group.

In some embodiments, T is a branching group which is trivalent, tetravalent or pentavalent. In some embodiments, T is a branching group which is trivalent. Therefore, in some embodiments, T is a trivalent branching group. In some embodiments, T is a branching group which is tetravalent. Therefore, in some embodiments, T is a tetravalent branching group.

In some embodiments, T is at least tetravalent further comprising a fourth terminal functionality to bind to a detection moiety (D) via a linker LD. In some embodiments, the fourth terminal functionality binds to L^{D} (or alternatively D) to form an amide group, a urea group, a thiourea group or a thioamide group.

Accordingly, in some embodiments, T is a tetravalent branching group and the compound of Formula I further includes a detection moiety (D) which is conjugated to T via a linker L^{D}, and the conjugate of Formula I is a conjugate of Formula I(a):

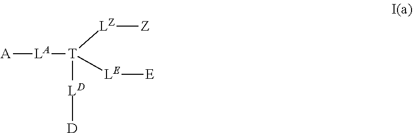

wherein:
T is a tetravalent branching group,
Z, A, E, L^{E}, L^{A}, L^{Z} are as defined in Formula I,
D is a detection moiety, wherein D is conjugated to T via a linker L^{D}.

In some embodiments, at least one of L^{A} and L^{E} is a cleavable linker.

In some embodiments, D comprises an aromatic ring. In some embodiments, the aromatic ring is a benzene ring. In some embodiments, D comprises a tyrosine (Tyr) residue, a tryptophan (Trp) residue or a phenylalanine (Phe) residue. In some embodiments, D is detectable by a UV detector. In some embodiments, D is detectable by HPLC.

In some embodiments, D is

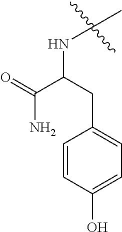 [SMILES: *NC(Cc1ccc(O)cc1)C(N)=O]

or the R- or S- stereoisomer thereof.

In some embodiments, L^{D} is a cleavable linker, direct bond or a non-cleavable linker.

In some embodiments, L^{D} is selected from the group consisting of amine bonds, ether bonds, thioether bonds, amide bonds, thioamide, urea or thiourea bonds.

In some embodiments, T comprises one or more amino acid residues derived from lysine, ornithine, homo-lysine, 2,3-diaminopropionic acid (DAP), 2,4-diaminobutyric acid (DAB), cysteine, homo-cysteine, and/or glutamine.

In some embodiments, T comprises one or more amino acid residues derived from lysine,

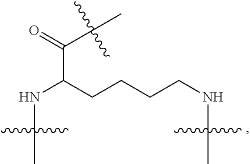 [SMILES: *NCCCCC(N*)C(*)=O]

DAP and/or DAB. In some embodiments, T comprises one or more amino acid residues derived from lysine,

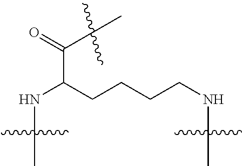 [SMILES: *NCCCCC(N*)C(*)=O]

and/or DAB. In some embodiments, T is one or more amino acid residues derived from DAB. In some embodiments, T is one or more amino acid residues derived from lysine,

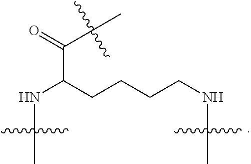 [SMILES: *NCCCCC(N*)C(*)=O]

Therefore, in some embodiments, T is an amino acid residue derived from lysine,

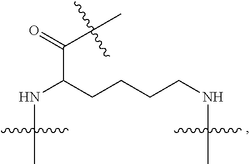 [SMILES: *NCCCCC(N*)C(*)=O]

and the conjugate of Formula I is a conjugate of Formula I-B, a conjugate of Formula I-C, a conjugate of Formula I-B′, or a conjugate of Formula I-C′,

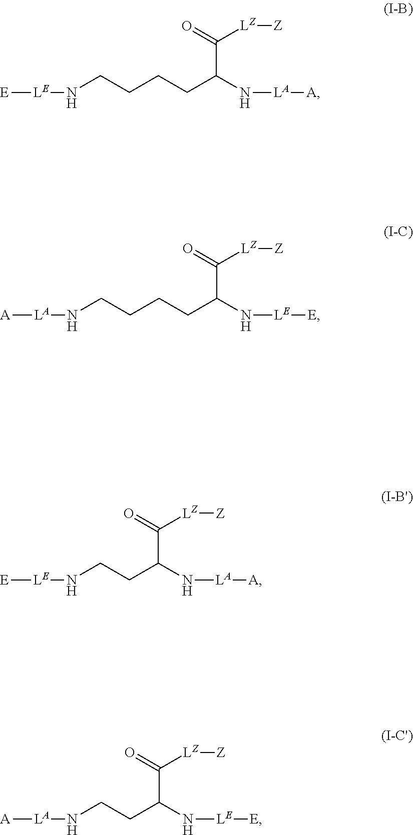

and wherein A, L^{A}, E, L^{E}, Z and L^{Z} are as defined in Formula I.

In some embodiments, the conjugate of Formula I is a conjugate of Formula I-B or a conjugate of Formula I-C:

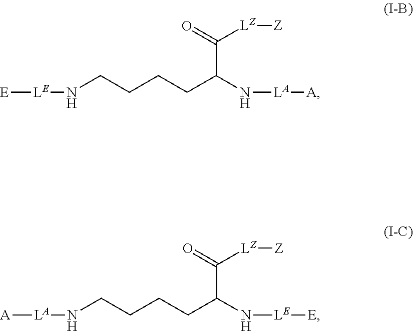

wherein A, L^{A}, E, L^{E}, Z and L^{Z} are as defined in Formula I.

In some embodiments, T comprises at least two amino acid residues derived from lysine, ornithine, homo-lysine, 2,3-diaminopropionic acid (DAP), 2,4-diaminobutyric acid (DAB), cysteine, homo-cysteine, glutamine, glutamic acid, asparagine, or aspartic acid.

In some embodiments, T is a tetravalent branching group that comprises two amino acid residues derived from lysine, DAP and/or DAB. In some embodiments, T is a tetravalent branching group that comprises two amino acid residues derived from lysine, having the following structure,

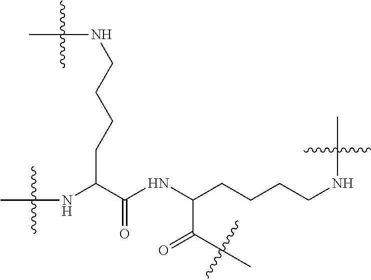 [SMILES: *NCCCCC(NC(=O)C(CCCCN*)N*)C(*)=O]

In an exemplary embodiment, T is a tetravalent branching group that comprises two amino acid residues derived from lysine, and the conjugate of Formula I is a conjugate of Formula I(a) is a conjugate of Formula I(a)-B

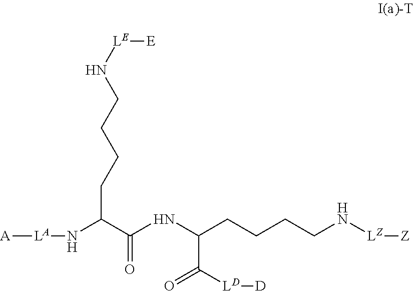

wherein A, L^{A}, E, L^{E}, D, L^{E}, Z and L^{Z} are as defined in Formula I(a).

In some embodiments, at least one of L^{A} and L^{E} is a cleavable linker.

In an exemplary embodiment, the albumin binding group (A) is selected from the group consisting of unsubstituted or substituted C(O)C₁₂₋₁₈alkyleneCOOH and C(O)C₁₂₋₁₈alkyl. In an exemplary embodiment, the albumin binding group (A) is selected from the group consisting of unsubstituted or substituted C(O)C₁₂₋₁₈alkyleneCOOH.

In an exemplary embodiment, the albumin binding group (A) is selected from the group consisting of unsubstituted or substituted C(O)C₁₂₋₁₈alkyleneCOOH, Z is an antibody or antibody mimetic and T is an amino acid residue derived from lysine,

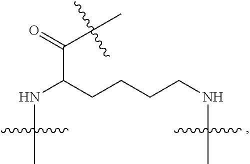 [SMILES: *NCCCCC(N*)C(*)=O]

DAP or DAB. In some embodiments, the albumin binding group (A) is selected from the group consisting of unsubstituted or substituted C(O)C₁₂₋₁₈alkyleneCOOH, Z is an antibody or antibody mimetic and T is an amino acid residue derived from lysine,

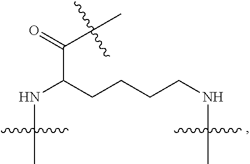 [SMILES: *NCCCCC(N*)C(*)=O]

or DAB. Therefore, in some embodiments, the conjugate of Formula I is a conjugate of Formula I-A′(a), a conjugate of Formula I-A′(b), a conjugate of Formula I-A′(c), a conjugate of Formula I-A′(d), and conjugate of Formula I(a)-A

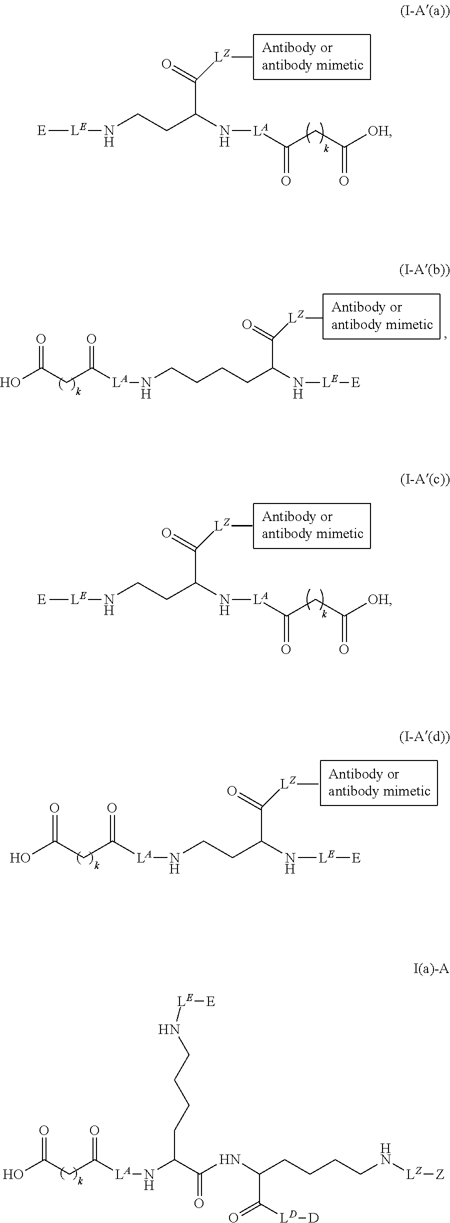

wherein in the Formulae of Formula I-A(a), Formula I-A(b), Formula I-A(c), Formula I-A(d) and Formula I(a)-A,
E, T, L^{A}, L^{E} and L^{Z} are as defined in Formula I,
L^{D} and D are as defined in Formula I(a); and
k is an integer from 10 to 20.

In some embodiments, at least one of L^{A} and L^{E} is a cleavable linker.

In some embodiments, k in the Formulae of Formula I-A(a), Formula I-A(b), Formula I-A(c), Formula I-A(d) and Formula I(a)-A is 14 to 18. In some embodiments, k in the Formulae of Formula I-A(a), Formula I-A(b), Formula I-A(c) Formula I-A(d) and Formula I(a)-A is 16 to 18. In some embodiments, k in the Formulae of Formula I-A(a), Formula I-A(b), Formula I-A(d) and Formula I(a)-A is 16 or 18. In some embodiments, E in the Formulae of Formula I-A(a), Formula I-A(b), Formula I-A(c), Formula I-A(d) and Formula I(a)-A is 14 to 18. In some embodiments, E in the Formulae of Formula I-A(a), Formula I-A(b), Formula I-A(c), Formula I-A(d) and Formula I(a)-A) is 16 or 18. In some embodiments, E in the Formulae of Formula I-A(a), Formula I-A(b), Formula I-A(d) and Formula I(a)-A is a chelating group derived from a chelating agent selected from the group consisting of DOTA and DOTAGA.

In some embodiments, L^{Z} of the conjugate is selected from the group consisting of a direct bond or a non-cleavable linker. In some embodiments of the conjugates, L^{Z} is a direct bond. In some embodiments, L^{Z} is a non-cleavable linker.

In some embodiments, L^{Z} conjugate is a non-cleavable linker comprising one or more non-cleavable moieties that resist degradation. In some embodiments, the one or more non-cleavable moieties that resist degradation are selected from the group consisting of amine bonds, ether bonds, thioether bonds, amide bonds, thioamide, urea and thiourea bonds.

In some embodiments, L^{Z} conjugate comprises one or more groups selected from the group consisting of

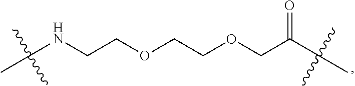 [SMILES: *NCCOCCOCC(*)=O]

R¹NC₁₋₂₀alkyleneNR², C(O)C₁₋₂₀alkyleneC(O), R¹NC₁₋₂₀alkyleneC(O), or C(O)C₁₋₂₀alkyleneNR², wherein each R¹ and R² is independently selected from the group consisting of H or C₁₋₂alkyl.

In some embodiments, one of L^{A} and L^{E} of the conjugate is a cleavable linker and the other is a direct bond or a non-cleavable linker. In some embodiments, one of L^{A} and L^{E} is a direct bond and the other is a cleavable linker. In some embodiments, one of L^{A} and L^{E} is a non-cleavable linker and the other is a cleavable linker. In some embodiments, L^{A} and L^{E} are both cleavable linkers. In some embodiments, L^{A} is a cleavable linker and L^{E} is a direct bond or a non-cleavable linker. In some embodiments, L^{A} and is a cleavable linker and L^{E} is a direct bond. In some embodiments, L^{A} and is a cleavable linker and L^{E} is a non-cleavable linker. In some embodiments, L^{E} and is a cleavable linker and L^{A} is a direct bond. In some embodiments, L^{E} and is a cleavable linker and L^{A} is a non-cleavable linker.

In some embodiments, each non-cleavable linker independently comprises one or more groups each comprising one or more non-cleavable moieties. In some embodiments, when L^{Z} is a non-cleavable linker and/or when one of L^{A} and L^{E} is a non-cleavable linker, each non-cleavable linker independently comprises one or more non-cleavable moieties that resist degradation. In some embodiments, the one or more non-cleavable moieties that resist degradation are selected from the group consisting of amine bonds, ether bonds, thioether bonds, amide bonds, thioamide, urea and thiourea bonds.

In some embodiments, each non-cleavable linker independently comprises one or more groups selected from the group consisting of R¹NC₁₋₂₀alkyleneNR², R¹NC₁₋₂₀alkenyleneNR² C(O)C₁₋₂₀alkyleneC(O), C(O)C₁₋₂₀alkenyleneC(O), R¹NC₁₋₂₀alkyleneC(O), R¹NC₁₋₂₀alkenyleneC(O), C(O)C₁₋₂₀alkyleneNHR², C(O)C₁₋₂₀alkenyleneNR², C(S)C₁₋₂₀alkyleneC(S), C(S)C₁₋₂₀alkenyleneC(S), C(S)C₁₋₂₀alkyleneC(O), C(S)C₁₋₂₀alkenyleneC(O), C(O)C₁₋₂₀alkyleneC(S), C(O)C₁₋₂₀alkenyleneC(S), C(O)C₁₋₂₀alkyleneO, C(O)C₁₋₂₀alkenyleneO, R¹NC₁₋₂₀alkenyleneC(S), C(S)C₁₋₂₀alkyleneNR² and C(S)C₁₋₂₀alkenyleneNR², each of which is independently and optionally interrupted by one or more of S, O, NH, N(C₁₋₆alkyl), C(O), C(O)NH, NHC(O), C(S)NH, NHC(S), NHC(O)NH, NHC(S)NH, NHC(NH), NHC(NC₁₋₄alkyl), C(NH)NH, C(NC₁₋₄alkyl)NH, NC₄₋₁₀cycloalkyl, C₄₋₁₀heterocycloalkyl, C₆₋₁₀aryl and C₅₋₁₀heteroaryl, and wherein each alkyl, alkylene and alkenylene is optionally substituted with one or more substituents selected from the group consisting of halo, COOH, C₁₋₆alkyl, OH, OC₁₋₆alkyl, SH, SC₁₋₆alkyl, NR³R⁴, C₁₋₄alkyleneOH, C₁₋₄alkyleneOC₁₋₄alkyl and C₁₋₄alkyleneNR³R⁴, wherein each R¹, R², R³ and R⁴ is independently selected from the group consisting of H and C₁₋₄alkyl.

In some embodiments, each non-cleavable linker independently comprises one or more groups selected from the group consisting of HNC₁₋₂₀alkyleneNH, HNC₁₋₂₀alkenyleneNH, C(O)C₁₋₂₀alkyleneC(O), C(O)C₁₋₂₀alkenyleneC(O), HNC₁₋₂₀alkyleneC(O), HNC₁₋₂₀alkenyleneC(O), C(O)C₁₋₂₀alkyleneNH, C(O)C₁₋₂₀alkenyleneNH, C(S)C₁₋₂₀alkyleneC(S), C(S)C₁₋₂₀alkenyleneC(S), C(S)C₁₋₂₀alkyleneC(O), C(S)C₁₋₂₀alkenyleneC(O), C(O)C₁₋₂₀alkyleneC(S), and C(O)C₁₋₂₀alkenyleneC(S), each of which is independently and optionally interrupted by one or more of S, O, NH, N(C₁₋₆alkyl), C(O), C(O)NH, NHC(O), C(S)NH, NHC(S), NHC(O)NH, NHC(S)NH, NHC(NH), NHC(NC₁₋₄alkyl), C(NH)NH, C(NC₁₋₄alkyl)NH, NC₄₋₁₀cycloalkyl, C₄₋₁₀heterocycloalkyl, C₆₋₁₀aryl and C₅₋₁₀heteroaryl, and wherein each alkyl, alkylene and alkenylene is optionally substituted with one or more substituents selected from the group consisting of halo, COOH, C₁₋₆alkyl, OH, OC₁₋₆alkyl, SH, SC₁₋₆alkyl, NH₂, NHC₁₋₄alkyl and N(C₁₋₄alkyl)₂.

In some embodiments, each non-cleavable linker independently comprises one or more groups selected from the group consisting of R¹NC₁₋₂₀alkyleneNR², R¹NC₁₋₂₀alkenyleneNR², C(O)C₁₋₂₀alkyleneC(O), C(O)C₁₋₂₀alkenyleneC(O), R¹NC₁₋₂₀alkyleneC(O), R¹NC₁₋₂₀alkenyleneC(O), C(O)C₁₋₂₀alkyleneNR², and C(O)C₁₋₂₀alkenyleneNR², each of which is independently and optionally interrupted by one or more of S, O, NH, N(C₁₋₆alkyl), C(O), C(O)NH, NHC(O), NHC(O)NH, NHC(S)NH, NC₄. 6cycloalkyl, C₄₋₆heterocycloalkyl, C₆₋₁₀aryl and C₅₋₁₀heteroaryl, and wherein each alkyl, alkylene and alkenylene is optionally substituted with one or more substituents selected from the group consisting of halo, COOH, C₁₋₆alkyl, OH, OC₁₋₆alkyl, SH, SC₁₋₆alkyl, NR³R⁴, C₁₋₄alkyleneOH, C₁₋₄alkyleneOC₁₋₄alkyl and C₁₋₄alkyleneNR³R⁴ wherein each R¹, R², R³ and R⁴ is independently selected from the group consisting of H and C₁₋₄alkyl.

In some embodiments, each non-cleavable linker independently comprises one or more groups selected from the group consisting of HNC₁₋₂₀alkyleneNH, HNC₁₋₂₀alkyleneNH, C(O)C₁₋₂₀alkyleneC(O), C(O)C₁₋₂₀alkyleneC(O), HNC₁₋₂₀alkyleneC(O), HNC₁₋₂₀alkenyleneC(O), C(O)C₁₋₂₀alkyleneNH, and C(O)C₁₋₂₀alkenyleneNH, each of which is independently and optionally interrupted by one or more of S, O, NH, N(C₁₋₆alkyl), C(O), C(O)NH, NHC(O), NHC(O)NH, NHC(S)NH, NC₄₋₆cycloalkyl, C₄₋₆heterocycloalkyl, C₆₋₁₀aryl and C₅₋₁₀heteroaryl, and wherein each alkyl, alkylene and alkenylene is optionally substituted with one or more substituents selected from the group consisting of halo, COOH, C₁₋₆alkyl, OH, OC₁₋₆alkyl, SH, SC₁₋₆alkyl, NH₂, NHC₁₋₄alkyl and N(C₁₋₄alkyl)₂.

In some embodiments, each non-cleavable linker independently comprises one or more groups selected from the group consisting of R¹NC₁₋₂₀alkyleneNR², C(O)C₁₋₂₀alkyleneC(O), R¹NC₁₋₂₀alkyleneC(O), and C(O)C₁₋₂₀alkyleneNR², each of which is independently and optionally interrupted by one or more of S, O, C(O)NH, NHC(O), NC₄₋₆ cycloalkyl, C₄₋₆heterocycloalkyl, and each alkyl, and alkylene is optionally substituted with one or more substituents selected from the group consisting of halo, COOH, C₁₋₆alkyl, OH, OC₁₋₆alkyl, SH, SC₁₋₆alkyl, NR³R⁴, C₁₋₄alkyleneOH, C₁₋₄alkyleneOC₁₋₄alkyl and C₁₋₄alkyleneNR³R⁴ wherein each R¹, R², R³ and R⁴ is independently selected from the group consisting of H and C₁₋₄alkyl.

In some embodiments, each non-cleavable linker independently comprises one or more groups selected from the group consisting of HNC₁₋₂₀alkyleneNH, C(O)C₁₋₂₀alkyleneC(O), HNC₁₋₂₀alkyleneC(O), and C(O)C₁₋₂₀alkyleneNH, the latter 4 groups being optionally interrupted by one or more of S, O, C(O)NH, NHC(O), NC₄₋₆cycloalkyl, C₄₋₆heterocycloalkyl, and each alkyl, alkylene and alkenylene is optionally and independently substituted with one or more substituents selected from the group consisting of halo, COOH, C₁₋₆alkyl, OH, OC₁₋₆alkyl, SH, SC₁₋₆alkyl, NH₂, NHC₁₋₄alkyl and N(C₁₋₄alkyl)₂.

In some embodiments, each non-cleavable linker independently comprises one or more groups selected from the group consisting of R¹NC₁₋₂₀alkyleneNR², C(O)C₁₋₂₀alkyleneC(O), R¹NC₁₋₂₀alkyleneC(O), and C(O)C₁₋₂₀alkyleneNR², which are interrupted by one or more of S, O, C(O)NH, NHC(O), C₄₋₆cycloalkyl and C₄₋₆heterocycloalkyl, and wherein each alkyl and alkylene is optionally substituted with one or more substituents selected from the group consisting of halo, COOH, NR³R⁴ and C₁₋₄alkyleneNR³R⁴ wherein each R¹, R², R³ and R⁴ is independently selected from the group consisting of H and C₁₋₄alkyl.

In some embodiments, each non-cleavable linker independently comprises one or more groups selected from the group consisting of R¹NC₁₋₂₀alkyleneNR², C(O)C₁₋₂₀alkyleneC(O), R¹NC₁₋₂₀alkyleneC(O), and C(O)C₁₋₂₀alkyleneNR², which are interrupted by one or more of S, O, C(O)NH, NHC(O), C₄₋₆cycloalkyl and C₄₋₆heterocycloalkyl, wherein each R¹ and R² is independently selected from the group consisting of H and C₁₋₄alkyl.

In some embodiments, each non-cleavable linker independently comprises one or more groups selected from the group consisting of HNC₁₋₂₀alkyleneNH, C(O)C₁₋₂₀alkyleneC(O), HNC₁₋₂₀alkyleneC(O), and C(O)C₁₋₂₀alkyleneNH, each of which is independently and optionally interrupted by one or more of S, O, C(O)NH, NHC(O), C₄₋₆cycloalkyl and C₄₋₆heterocycloalkyl.

In some embodiments, R¹, R², R³ and R⁴ are independently selected from the group consisting of H, CH₃, CH₂CH₃, CH₂CH₂CH₃, CH(CH₃)₂, and C(CH₃)₃. In some embodiments, R¹, R², R³ and R⁴ are independently selected from the group consisting of H and C₁₋₃alkyl. In some embodiments, R¹, R², R³ and R⁴ are independently selected from the group consisting of H, CH₃, CH₂CH₃, and CH(CH₃)₂. In some embodiments, R¹, R², R³ and R⁴ are independently selected from the group consisting of H, and CH₃.

In some embodiments, one or both of L^{A} and L^{E} are cleavable linkers, and each cleavable linker independently comprises at least one group comprising one to four cleavable moieties or at least two groups comprising one to four cleavable moieties which connect to form a cleavable moiety. In some embodiments, one or both of L^{A} and L^{E} is a cleavable linker, and each cleavable linker independently comprises two to four groups each comprising one or two cleavable moieties and/or which connect to form a cleavable moiety.

In some embodiments, one or both of L^{A} and L^{E} is a cleavable linker, and each cleavable linker independently comprises two groups which each comprise one or two cleavable moieties and/or which connect to form cleavable moieties. In some embodiments, one or both of L^{A} and L^{E} is a cleavable linker, and each cleavable linker independently comprises one group comprising one or two cleavable moieties. In some embodiments, L^{A} and L^{E} are both cleavable linkers each comprising one or two groups which each comprise two cleavable moieties and/or which further connect to form a cleavable moiety.

In some embodiments, the cleavable linker comprises one or more cleavable moieties selected from the group consisting of S—S, C(O)O, OC(O), OC(O)O, OC(O)NH, NHC(O)O, SC(O), C(O)S, C═NNH, C═NO, NC₁₋₄alkylC(O)O, NHOC(O)NH, NC₁-4alkylOC(O), and an enzymatically cleavable peptide sequence. In some embodiments, the cleavable linker comprises one or more cleavable moieties of S—S, C(O)O, and/or OC(O).

In some embodiments, one or both of L^{A} and L^{E} are cleavable linkers and each cleavable linker independently comprises one or more groups selected from the group consisting of R⁵NC₁₋₂₀alkyleneNR⁶, R⁵NC₁₋₂₀alkenyleneNR⁵, C(O)C₁₋₂₀alkyleneC(O), C(O)C₁₋₂₀alkenyleneC(O), R⁵NC₁₋₂₀alkyleneC(O), R⁵NC₁₋₂₀alkenyleneC(O), C(O)C₁₋₂₀alkyleneNR⁶, C(O)C₁₋₂₀alkenyleneNR⁶, C(S)C₁₋₂₀alkyleneC(S), C(S)C₁₋₂₀alkenyleneC(S), C(S)C₁₋₂₀alkyleneC(O), C(S)C₁₋₂₀alkenyleneC(O), C(O)C₁₋₂₀alkyleneC(S), C(O)C₁₋₂₀alkenyleneC(S), SC₁₋₂₀alkyleneS, SC₁₋₂₀alkenyleneS, SC₁₋₂₀alkyleneNR⁶, SC₁₋₂₀alkenyleneNR⁶, RNC₁₋₂₀alkyleneS, R⁵NC₁₋₂₀alkenyleneS, R⁵NC₁₋₂₀alkyleneO, R⁵NC₁₋₂₀alkenyleneO, OC₁₋₂₀alkyleneNR⁶, OC₁₋₂₀alkenyleneNR⁶, SC₁₋₂₀alkyleneO, SC₁₋₂₀alkenyleneO, OC₁₋₂₀alkyleneS, and OC₁₋₂₀alkenyleneS, C(O)C₁₋₂₀alkyleneO, C(O)C₁₋₂₀alkenyleneO, OC₁₋₂₀alkyleneC(O), OC₁₋₂₀alkenyleneC(O), C(O)C₁₋₂₀alkyleneS, C(O)C₁₋₂₀alkenyleneS, SC₁₋₂₀alkyleneC(O), SC₁₋₂₀alkenyleneC(O), R⁵NC₁₋₂₀alkyleneC(S), R⁵NC₁₋₂₀alkenyleneC(S), C(S)C₁₋₂₀alkyleneNR⁶, C(S)C₁₋₂₀alkenyleneNR⁶, C(S)C₁₋₂₀alkyleneO, C(S)C₁₋₂₀alkenyleneO, OC₁₋₂₀alkyleneC(S), OC₁₋₂₀alkenyleneC(S), SC₁₋₂₀alkyleneC(S), SC₁₋₂₀alkenyleneC(S), OC₁₋₂₀alkyleneO, OC₁₋₂₀alkenyleneCO, SC₁₋₂₀alkyleneS, SC₁₋₂₀alkenyleneS, each of which is independently and optionally interrupted by one or more of S—S, C(O)O, OC(O), OC(O)O, OC(O)NH, NHC(O)O, SC(O), C(O)S, NC₁₋₄alkylC(O)O, NHOC(O)NH, NC₁₋₄alkylOC(O), C═NNH, C═NNH₂, C═NOH, C═NO, NH—NH, NH—NC₁₋₄alkyl, NC₁₋₄alkyl-NH, NC₁₋₄alkylNC₁₋₄alkyl, S, O, NH, N(C₁₋₆alkyl), C(O), C(O)NH, NHC(O), NHC(O)NH, NHC(S)NH, C(S)NH, NHC(S), NHC(NH), NHC(NC₁₋₄alkyl), C(NH)NH, C(NC₁₋₄alkyl)NH, NC₄₋₁₈cycloalkyl, C₄₋₁₀heterocycloalkyl, C₆₋₁₀aryl and C₅₋₁₀heteroaryl, and wherein each alkyl, alkylene and alkenylene is optionally substituted with one or more substituents selected from the group consisting of halo, COOH, C₁₋₆alkyl, OH, OC₁₋₆alkyl, SH, SC₁₋₆alkyl, NR⁷R⁷, C₁₋₄alkyleneOH, C₁₋₄alkyleneOC₁₋₄alkyl and C₁₋₄alkyleneNR⁷R⁸ wherein R⁵, R⁶, R⁷ and R⁸ are independently selected from the group consisting of H and C₁₋₄alkyl.

In some embodiments, one or both of L^{A} and L^{E} is a cleavable linker and each cleavable linker independently comprises one or more groups selected from the group consisting of HNC₁₋₂₀alkyleneNH, HNC₁₋₂₀alkenyleneNH, C(O)C₁₋₂₀alkyleneC(O), C(O)C₁₋₂₀alkenyleneC(O), HNC₁₋₂₀alkyleneC(O), HNC₁₋₂₀alkenyleneC(O), C(O)C₁₋₂₀alkyleneNH, C(O)C₁₋₂₀alkenyleneNH, C(S)C₁₋₂₀alkyleneC(S), C(S)C₁₋₂₀alkenyleneC(S), C(S)C₁₋₂₀alkyleneC(O), C(S)C₁₋₂₀alkenyleneC(O), C(O)C₁₋₂₀alkyleneC(S), C(O)C₁₋₂₀alkenyleneC(S), SC₁₋₂₀alkyleneS, SC₁₋₂₀alkenyleneS, SC₁₋₂₀alkyleneNH, SC₁₋₂₀alkenyleneNH, HNC₁₋₂₀alkyleneS, HNC₁₋₂₀alkenyleneS, HNC₁₋₂₀alkyleneO, HNC₁₋₂₀alkenyleneO, OC₁₋₂₀alkyleneNH, OC₁₋₂₀alkenyleneNH, SC₁₋₂₀alkyleneO, SC₁₋₂₀alkenyleneO, OC₁₋₂₀alkyleneS, and OC₁₋₂₀alkenyleneS, each of which is independently and optionally interrupted by one or more of S—S, C(O)O, OC(O), OC(O)O, OC(O)NH, NHC(O)O, SC(O), C(O)S, NC₁₋₄alkylC(O)O, NHOC(O)NH, NC₁₋₄alkylOC(O), C═NNH, C═NNH₂, C═NOH, C═NO, NH—NH, NH—NC₁₋₄alkyl, NC₁₋₄alkyl-NH, NC₁₋₄alkylNC₁₋₄alkyl, S, O, NH, N(C₁₋₆alkyl), C(O), C(O)NH, NHC(O), NHC(O)NH, NHC(S)NH, C(S)NH, NHC(S), NHC(NH), NHC(NC₁₋₄ alkyl), C(NH)NH, C(NC₁₋₄alkyl)NH, NC₄₋₁₈cycloalkyl, C₄₋₁₀heterocycloalkyl, C₆₋₁₀aryl and C₅₋₁₀heteroaryl, and wherein each alkyl, alkylene and alkenylene is optionally substituted with one or more substituents selected from the group consisting of halo, COOH, C₁₋₆(alkyl, OH, OC₁₋₆alkyl, SH, SC₁₋₆alkyl, NH₂, NHC₁₋₄alkyl and N(C₁₋₄alkyl)₂, and wherein R⁵, R⁶, R⁷ and R⁸ are independently selected from the group consisting of H and C₁₋₄alkyl

In some embodiments, one or both of L^{A} and L^{E} of the conjugate is a cleavable linker, and each cleavable linker independently comprises one or more groups selected from the group consisting of R⁵NC₁₋₂₀alkyleneNR⁶, R⁵NC₁₋₂₀alkenyleneNR⁶, C(O)C₁₋₂₀alkyleneC(O), C(O)C₁₋₂₀alkenyleneC(O), R⁵NC₁₋₂₀alkyleneC(O), R⁵NC₁₋₂₀alkenyleneC(O), C(O)C₁₋₂₀alkyleneNR⁶, C(O)C₁₋₂₀alkenyleneNR⁶, C(S)C₁₋₂₀alkyleneC(S), C(S)C₁₋₂₀alkenyleneC(S), C(S)C₁₋₂₀alkyleneC(O), C(S)C₁₋₂₀alkenyleneC(O), C(O)C₁₋₂₀alkyleneC(S), SC₁₋₂₀alkyleneS, SC₁₋₂₀alkyleneNR⁶, R⁵NC₁₋₂₀alkyleneS, R⁵NC₁₋₂₀alkyleneO, R⁵NC₁₋₂₀alkenyleneO, OC₁₋₂₀alkyleneNR⁶, OC₁₋₂₀alkenyleneNR⁶, SC₁₋₂₀alkyleneO, OC₁₋₂₀alkyleneS, C(O)C₁₋₂₀alkyleneO, C(O)C₁₋₂₀alkenyleneO, OC₁₋₂₀alkyleneC(O) and OC₁₋₂₀alkenyleneC(O), each of which is independently and optionally interrupted by one or more of S—S, C(O)O, OC(O), OC(O)O, OC(O)NH, NHC(O)O, SC(O), C(O)S, NC₁₋₄alkylC(O)O, NHOC(O)NH, NC₁₋₄alkylOC(O), C═NNH, C═NNH₂, C═NOH, C═NO,), NH—NH, S, O, NH, N(C₁₋₆alkyl), C(O), C(O)NH, NHC(O), NHC(O)NH, NHC(S)NH, C(S)NH, NHC(S), NC₄₋₁₈cycloalkyl, C₄₋₁₀heterocycloalkyl, C₆₋₁₀aryl and C₅₋₁₀heteroaryl, and wherein each alkyl, alkylene and alkenylene is optionally substituted with one or more substituents selected from the group consisting of halo, COOH, C₁₋₆alkyl, OH, OC₁₋₆alkyl, SH, SC₁₋₆alkyl, NR⁷R⁷, C₁₋₄alkyleneOH, C₁₋₄alkyleneOC₁₋₄alkyl and C₁₋₄alkyleneNR⁷R⁸, wherein R⁵, R⁶, R⁷ and R⁸ are independently selected from the group consisting of H and C₁₋₄alkyl.

In some embodiments, one or both of L^{A} and L^{E} is a cleavable linker, and each cleavable linker independently comprises one or more groups selected from the group consisting of HNC₁₋₂₀alkyleneNH, HNC₁₋₂₀alkenyleneNH, C(O)C₁₋₂₀alkyleneC(O), C(O)C₁₋₂₀alkenyleneC(O), HNC₁₋₂₀alkyleneC(O), HNC₁₋₂₀alkenyleneC(O), C(O)C₁₋₂₀alkyleneNH, C(O)C₁₋₂₀alkenyleneNH, C(S)C₁₋₂₀alkyleneC(S), C(S)C₁₋₂₀alkenyleneC(S), C(S)C₁₋₂₀alkyleneC(O), C(S)C₁₋₂₀alkenyleneC(O), C(O)C₁₋₂₀alkyleneC(S), SC₁₋₂₀alkyleneS, SC₁₋₂₀alkyleneNH, HNC₁₋₂₀alkyleneS, HNC₁₋₂₀alkyleneO, HNC₁₋₂₀alkenyleneO, OC₁₋₂₀alkyleneNH, OC₁₋₂₀alkenyleneNH, SC₁₋₂₀alkyleneO and OC₁₋₂₀alkyleneS, each of which is independently and optionally interrupted by one or more of S—S, C(O)O, OC(O), OC(O)O, OC(O)NH, NHC(O)O, SC(O), C(O)S, NC₁₋₄alkylC(O)O, NHOC(O)NH, NC₁₋₄ alkylOC(O), C═NNH, C═NNH₂, C═NOH, C═NO,), NH—NH, S, O, NH, N(C₁₋₆alkyl), C(O), C(O)NH, NHC(O), NHC(O)NH, NHC(S)NH, C(S)NH, NHC(S), NC₄₋₁₈cycloalkyl, C₄₋₁₀heterocycloalkyl, C₆₋₁₀aryl and C₅₋₁₀heteroaryl, and each alkyl, alkylene and alkenylene is optionally and independently substituted with one or more substituents selected from the group consisting of halo, COOH, C₁₋₆alkyl, OH, OC₁₋₆alkyl, SH, SC₁₋₆alkyl, NH₂, NHC₁₋₄alkyl and N(C₁₋₄ alkyl)₂, and wherein R⁵, R⁶, R⁷ and R⁸ are independently selected from the group consisting of H and C₁₋₄alkyl.

In some embodiments, one or both of L^{A} and L^{E} is a cleavable linker, and each cleavable linker independently comprises one or more groups selected from the group consisting of R⁵NC₁₋₂₀alkyleneNR⁶, R⁵NC₁₋₂₀alkenyleneNR⁶, C(O)C₁₋₂₀alkyleneC(O), C(O)C₁₋₂₀alkenyleneC(O), R⁵NC₁₋₂₀alkyleneC(O), R⁵NC₁₋₂₀alkenyleneC(O), C(O)C₁₋₂₀alkyleneNR⁶, C(O)C₁₋₂₀alkenyleneNR⁶, R⁵NC₁₋₂₀alkyleneO, R⁵NC₁₋₂₀alkenyleneO, OC₁₋₂O alkyleneNR⁶, OC₁₋₂₀alkenyleneNR⁶, OC₁₋₂₀alkyleneS, C(O)C₁₋₂₀alkyleneO, C(O)C₁₋₂₀alkenyleneO, OC₁₋₂₀alkyleneC(O) and OC₁₋₂₀alkenyleneC(O), each of which is independently and optionally interrupted by one or more of S—S, C(O)O, OC(O), OC(O)O, OC(O)NH, NHC(O)O, SC(O), C(O)S, NC₁₋₄alkylC(O)O, NHOC(O)NH, NC₁₋₄alkylOC(O), S, O, NH, N(C₁₋₆alkyl), C(O), C(O)NH, NHC(O), NHC(O)NH, NHC(S)NH, C(S)NH, NHC(S) NC₄₋₁₈cycloalkyl, C₄₋₁₀heterocycloalkyl, C₆₋₁₀aryl and C₅₋₁₀heteroaryl, and wherein each alkyl, alkylene and alkenylene is optionally substituted with one or more substituents selected from the group consisting of halo, COOH, C₁₋₆alkyl, OH, OC₁₋₆alkyl, SH, SC₁₋₆alkyl, NR⁷R⁷, C₁₋₄alkyleneOH, C₁₋₄alkyleneOC₁₋₄alkyl and C₁₋₄alkyleneNR⁷R⁸ wherein R⁵, R⁶, R⁷ and R⁸ are independently selected from the group consisting of H and C₁₋₄alkyl.

In some embodiments, one or both of L^{A} and L^{E} is a cleavable linker, and each cleavable linker independently comprises one or more groups selected from the group consisting of HNC₁₋₂₀alkyleneNH, HNC₁₋₂₀alkenyleneNH, C(O)C₁₋₂₀alkyleneC(O), C(O)C₁₋₂₀alkenyleneC(O), HNC₁₋₂₀alkyleneC(O), HNC₁₋₂₀alkenyleneC(O), C(O)C₁₋₂₀alkyleneNH, C(O)C₁₋₂₀alkenyleneNH, HNC₁₋₂₀alkyleneO, HNC₁₋₂₀alkenyleneO, OC₁₋₂₀alkyleneNH, and OC₁₋₂₀alkenyleneNH, each of which is independently and optionally interrupted by one or more of S—S, C(O)O, OC(O), OC(O)O, OC(O)NH, NHC(O)O, SC(O), C(O)S, NC₁₋₄ alkylC(O)O, NHOC(O)NH, NC₁₋₄alkylOC(O), S, O, NH, N(C₁₋₆alkyl), C(O), C(O)NH, NHC(O), NHC(O)NH, NHC(S)NH, C(S)NH, NHC(S) NC₄₋₁₈cycloalkyl, C₄₋₁₀heterocycloalkyl, C₆₋₁₀aryl and C₅₋₁₀heteroaryl, and wherein each alkyl, alkylene and alkenylene is optionally substituted with one or more substituents selected from the group consisting of halo, COOH, C₁₋₆alkyl, OH, OC₁₋₆alkyl, SH, SC₁₋₆alkyl, NH₂, NHC₁₋₄alkyl and N(C₁₋₄alkyl)₂.

In some embodiments, one or both of L^{A} and L^{E} of the conjugate is a cleavable linker, and each cleavable linker independently comprises one or more groups selected from the group consisting of R⁵NC₁₋₂₀alkyleneNR⁶, C(O)C₁₋₂₀alkyleneC(O), R⁵NC₁₋₂₀alkyleneC(O), C(O)C₁₋₂₀alkyleneNR⁶, R⁵NC₁₋₂₀alkyleneO, OC₁₋₂₀alkyleneNR⁶, C(O)C₁₋₂₀alkyleneO and OC₁₋₂₀alkyleneC(O), each of which is independently and optionally interrupted by one or more of S—S, C(O)O, OC(O), OC(O)O, OC(O)NH, NHC(O)O, SC(O), C(O)S, NC₁₋₄alkylC(O)O, NHOC(O)NH, NC₁₋₄alkylOC(O), S, O, NH, N(C₁₋₆alkyl), C(O), C(O)NH, NHC(O), NHC(O)NH, NHC(S)NH, C(S)NH, NHC(S), C₄₋₁₈cycloalkyl, C₄₋₁₀heterocycloalkyl, C₆₋₁₀aryl and C₅₋₁₀heteroaryl, and wherein each alkyl and alkylene is optionally substituted with one or more substituents selected from the group consisting of halo, COOH, C₁₋₆alkyl, OH, OC₁₋₆alkyl, SH, SC₁₋₆alkyl, NR⁷R⁷, C₁₋₄alkyleneOH, C₁₋₄alkyleneOC₁₋₄alkyl and C₁₋₄alkyleneNR⁷R⁸ wherein R⁵, R⁶, R⁷ and R⁸ are independently selected from the group consisting of H and C₁₋₄alkyl.. In some embodiments, one or both of L^{A} and L^{E} is a cleavable linker, and each cleavable linker independently comprises one or more groups selected from the group consisting of HNC₁₋₂₀alkyleneNH, C(O)C₁₋₂₀alkyleneC(O), HNC₁₋₂₀alkyleneC(O), C(O)C₁₋₂₀alkyleneNH, HNC₁₋₂₀alkyleneO, OC₁₋₂₀alkyleneNH, which are being optionally interrupted by one or more of S—S, C(O)O, OC(O), OC(O)O, OC(O)NH, NHC(O)O, SC(O), C(O)S, NC₁₋₄alkylC(O)O, NHOC(O)NH, NC₁₋₄alkylOC(O), S, O, NH, N(C₁₋₆alkyl), C(O), C(O)NH, NHC(O), NHC(O)NH, NHC(S)NH, C(S)NH, NHC(S), C₄₋₁₈cycloalkyl, C₄₋₁₀heterocycloalkyl, C₆₋₁₀aryl and C₅₋₁₀heteroaryl, and wherein each alkyl and alkylene is optionally substituted with one or more substituents selected from the group consisting of halo, COOH, C₁₋₆alkyl, OH, OC₁₋₆alkyl, SH, SC₁₋₆alkyl, NH₂, NHC₁₋₄alkyl and N(C₁₋₄ alkyl)₂.

In some embodiments, one or both of L^{A} and L^{E} of the conjugate is a cleavable linker, and each cleavable linker independently comprises one or more groups selected from the group consisting of R⁵NC₁₋₂₀alkyleneNR⁶, C(O)C₁₋₂₀alkyleneC(O), R⁵NC₁₋₂₀alkyleneC(O), C(O)C₁₋₂₀alkyleneNR⁶, R⁵NC₁₋₂₀alkyleneO, OC₁₋₂₀alkyleneNR⁶, C(O)C₁₋₂₀alkyleneO and OC₁₋₂₀alkyleneC(O) each of which is independently and optionally interrupted by one or more of S—S, C(O)O, OC(O), OC(O)O, OC(O)NH, SC(O), C(O)S, S, O, NH, N(C₁₋₆alkyl), C(O), C(O)NH, NHC(O), NHC(O)NH, NHC(S)NH, C(S)NH, NHC(S), C₄₋₁₈cycloalkyl, and C₄₋₁₀heterocycloalkyl, and wherein each alkyl and alkylene is optionally substituted with one or more substituents selected from the group consisting of halo, COOH, C₁₋₆alkyl, OH, OC₁₋₆alkyl, SH, SC₁₋₆alkyl, NR⁷R⁷, C₁₋₄alkyleneOH, and C₁₋₄alkyleneNR⁷R⁸

wherein R⁵, R⁶, R⁷ and R⁸ are independently selected from the group consisting of H and C₁₋₄alkyl.

In some embodiments, one or both of L^{A} and L^{E} of the conjugate is a cleavable linker, and each cleavable linker independently comprises one or more groups selected from the group consisting of HNC₁₋₂₀alkyleneNH, C(O)C₁₋₂₀alkyleneC(O), HNC₁₋₂₀alkyleneC(O), C(O)C₁₋₂₀alkyleneNH, HNC₁₋₂₀alkyleneO and OC₁₋₂₀alkyleneNH, each of which is independently and optionally interrupted by one or more of S—S, C(O)O, OC(O), OC(O)O, OC(O)NH, SC(O), C(O)S, S, O, NH, N(C₁₋₆alkyl), C(O), C(O)NH, NHC(O), NHC(O)NH, NHC(S)NH, C(S)NH, NHC(S), C₄₋₁₈cycloalkyl, and C₄₋₁₀heterocycloalkyl, and wherein each alkyl and alkylene is optionally substituted with one or more substituents selected from the group consisting of halo, COOH, C₁₋₆alkyl, OH, OC₁₋₆alkyl, SH, SC₁₋₆alkyl, NH₂, NHC₁₋₄alkyl and N(C₁₋₄alkyl)₂.

In some embodiments, one or both of L^{A} and L^{E} of the conjugate is a cleavable linker, and each cleavable linker independently comprises one or more groups selected from the group consisting of R⁵NC₁₋₂₀alkyleneNR⁶, C(O)C₁₋₂₀alkyleneC(O), R⁵NC₁₋₂₀alkyleneC(O), C(O)C₁₋₂₀alkyleneNR⁶, R⁵NC₁₋₂₀alkyleneO, OC₁₋₂₀alkyleneNR⁶, C(O)C₁₋₂₀alkyleneO and OC₁₋₂₀alkyleneC(O), each of which is independently and optionally interrupted by one or more of S—S, C(O)O, OC(O), S, O, C(O)NH, NHC(O), C₄₋₁₈cycloalkyl, and C₄₋₁₀heterocycloalkyl, and wherein each alkyl and alkylene is optionally substituted with one or more substituents selected from the group consisting of halo, COOH, C₁₋₆alkyl, OH, OC₁₋₆alkyl, SH, SC₁₋₆alkyl, NR⁷R⁷, C₁₋₄alkyleneOH, and C₁₋₄alkyleneNR⁷R⁸, wherein R⁵, R⁶, R⁷ and R⁸ are independently selected from the group consisting of H and C₁₋₄alkyl.

In some embodiments, one or both of L^{A} and L^{E} of the conjugate is a cleavable linker, and each cleavable linker independently comprises one or more groups selected from the group consisting of HNC₁₋₂₀alkyleneNH, C(O)C₁₋₂₀alkyleneC(O), HNC₁₋₂₀alkyleneC(O), C(O)C₁₋₂₀alkyleneNH, HNC₁₋₂₀alkyleneO and OC₁₋₂₀alkyleneNH, each of which is independently and optionally interrupted by one or more of S—S, C(O)O, OC(O), S, O, C(O)NH, NHC(O), C₄₋₁₈cycloalkyl, and C₄₋₁₀heterocycloalkyl, and wherein each alkyl, and alkylene is optionally substituted with one or more substituents selected from the group consisting of halo, COOH, C₁₋₆alkyl, OH, OC₁₋₆alkyl, SH, SC₁₋₆alkyl, NH₂, NHC₁₋₄alkyl and N(C₁₋₄alkyl)₂. In some embodiments, one or both of L^{A} and L^{E} is a cleavable linker, and each cleavable linker independently comprises one or more groups selected from the group consisting of R⁵NC₁₋₂₀alkyleneNR⁶, C(O)C₁₋₂₀alkyleneC(O), R⁵NC₁₋₂₀alkyleneC(O), C(O)C₁₋₂₀alkyleneNR⁶, R⁵NC₁₋₂₀alkyleneO, OC₁₋₂₀alkyleneNR⁶, C(O)C₁₋₂₀alkyleneO and OC₁₋₂₀alkyleneC(O), each of which is independently and optionally interrupted by one or more of S—S, C(O)O, OC(O), S, O, C(O)NH, NHC(O), C₄₋₁₈cycloalkyl, and C₄₋₁₀heterocycloalkyl, and each alkyl and alkylene is optionally substituted with one or more substituents selected from the group consisting of halo, COOH, NR⁷R⁷, and C₁₋₄alkyleneNR⁷R⁸, wherein R⁵, R⁶, R⁷ and R⁸ are independently selected from the group consisting of H and C₁₋₄alkyl. In some embodiments, one or both of L^{A} and L^{E} is a cleavable linker, and each cleavable linker independently comprises one or more groups selected from the group consisting of HNC₁₋₂₀alkyleneNH, C(O)C₁₋₂₀alkyleneC(O), HNC₁₋₂₀alkyleneC(O), C(O)C₁₋₂₀alkyleneNH, HNC₁₋₂₀alkyleneO and OC₁₋₂₀alkyleneNH, each of which is independently and optionally interrupted by one or more of S—S, C(O)O, OC(O), S, O, C(O)NH, NHC(O), C₄₋₁₈cycloalkyl, and C₄₋₁₀heterocycloalkyl.

In some embodiments, at least two groups connect to form a cleavable moiety. In some embodiments, two groups selected from the group consisting of amino acid residue, R⁵NC₁₋₂₀alkyleneO, OC₁₋₂₀alkyleneNR⁶, C(O)C₁₋₂₀alkyleneO and OC₁₋₂₀alkyleneC(O) connect to form a cleavable moiety selected from the group consisting of C(O)O and OC(O).

In some embodiments, an amino acid residue and one of R⁵NC₁₋₂₀alkyleneO, OC₁₋₂₀alkyleneNR⁶, C(O)C₁₋₂₀alkyleneO and OC₁₋₂₀alkyleneC(O) connect to form a cleavable moiety selected from the group consisting of C(O)O and OC(O). In some embodiments, an amino acid residue and one of C(O)C₁₋₂₀alkyleneO and OC₁₋₂₀alkyleneC(O) connect to form a cleavable moiety selected from the group consisting of C(O)O, S—S, and OC(O). In some embodiments, the OC₁₋₂₀alkyleneC(O) is selected from the group consisting of 4-hydroxybutanoic acid (4hBA), 4-hydroxypentanoic acid (4hPA), 5-hydroxypentanoic acid (5HPA) and 6-hydroxyhexanoic acid (6hHA).

In some embodiments, C₄₋₁₀cycloalkyl is selected from the group consisting of cyclopropyl, cyclobutyl, cyclopentyl, cyclohexyl, and cycloheptyl.

In some embodiments, C₄₋₁₀heterocycloalkyl is selected from the group consisting of azetidinyl, oxetanyl, tetrohydrofuranyl, tetrahydrothiophenyl, pyrrolidinyl, dihydropyrrolyl, imidazolidinyl, pyrazolidinyl, thiazolidinyl, isothiazolidinyl, dioxolanyl, dithiolanyl, 3,4,5,6-tetrahydro-1,2,4-triazinyl, dioxidothiomorpholino, tetrahydropyridinyl, dihydropyridinyl, dihydropyranyl, thianyl, piperidinyl, piperazinyl, tetrahydropyranyl, thiomorpholinyl, morpholinyl, dioxanyl, azepanyl, diazepanyl, oxepanyl and thiepanyl.

In some embodiments, the C₆₋₁₀aryl is selected from the group consisting of phenyl, indanyl or naphthyl.

In some embodiments, the C₅₋₁₁heteroaryl is selected from the group consisting of triazolyl, pyrrolyl, imidazolyl, oxazolyl, pyrazolyl, thiazolyl, pyridinyl, pyrazinyl, pyridazinyl and pyrimidinyl.

In some embodiments, one or both of L^{A} and L^{E} of the conjugate is a cleavable linker, and each cleavable linker independently comprises one or more amino acid residues.

In some embodiments, the amino acid residue in L^{A}, L^{E} and/or L^{Z} is derived from a naturally occurring amino acid or an unnaturally occurring amino acid.

In some embodiments, the naturally occurring amino acid is selected from the group consisting of, but are not limited to, alanine (Ala, A), arginine (Arg, R), asparagine (Asn, N), aspartic acid (Asp, D), cysteine (Cys, C), glutamine (Gln, Q), glutamic acid (Glu, E), γGlu

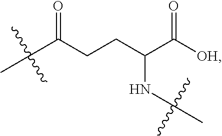 [SMILES: *NC(CCC(*)=O)C(=O)O]

glutamine (Gln), glycine (Gly, G), histidine (His, H), isoleucine (Ile, I), leucine (Leu, L), Lysine (Lys, K), εLys,

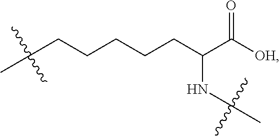 [SMILES: *CCCCCC(N*)C(=O)O]

methionine (Met, M), phenylalanine (Phe, F), proline (Pro, P), serine (Ser, S), threonine (Thr, T), tryptophan (Trp, W), tyrosine (Tyr, Y), valine (Val, V), pyrrolysine (Pyl, O), selenocycleine (Sec), and pyrroline-carboxy-lysine (PCL).

In some embodiments, the unnaturally occurring amino acid is selected from the group consisting of modified amino acids, β- amino acids, γ-amino acids, homo amino acids, N-methyl amino acids, alpha-methyl amino acids, des-amino amino acids and D enantiomer of the naturally occurring amino acids or the modified amino acids.

In some embodiments, the unnaturally occurring amino acid is one or more modified amino acid. In some embodiments, the one or more modified amino acids are selected from the group consisting of, but not limited to, hydroxyproline (Hyp), γ-carboxyglutamate, O-phosphoserine, azetidinecarboxylic acid, 2-aminoadipic acid, 3-aminoadipic acid, beta-alanine, aminopropionic acid, 2-aminobutyric acid (Aib), 4-aminobutyric acid, 6-aminocaproic acid, 2-aminoheptanoic acid, 2-aminoisobutyric acid, 3-aminoisobutyric acid, 2-aminopimelic acid, tertiary-butylglycine, 2,4-diaminoisobutyric acid, desmosine, 2,2′-diaminopimelic acid, 2,3-diaminoproprionic acid, N-ethylglycine, N-methylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, norleucine, ornithine, pentylglycine, pipecolic acid, thioproline, nitroarginine, 2,4-diaminobutyric acid (DAB), 2,3-diaminopropionic acid (DAP), methionine sulfoxide and methionine sulfone. .

In some embodiments, the amino acid residue is derived from the D enantiomer of the naturally occurring amino acids or the modified amino acids.

In some embodiments, the amino acid residue derived from Glu is connected through the α-amino and the α-carboxy or the α-amino and the γ-carboxy terminal. Therefore, in some embodiments, the amino acid residue derived from Glu is

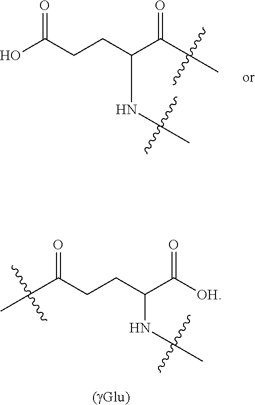 [SMILES: *NC(CCC(=O)O)C(*)=O ; *NC(CCC(*)=O)C(=O)O]

In some embodiments, the amino acid residue derived from Asp is connected through amino and the α-carboxy or is connected through the amino and the 3-carboxy terminal. In some embodiments, the amino acid residue derived from Lys is connected through α-amino and the α-carboxy or the α-amino and the s-amino terminal.

Therefore, in some embodiments, the amino acid residue is derived from Lys

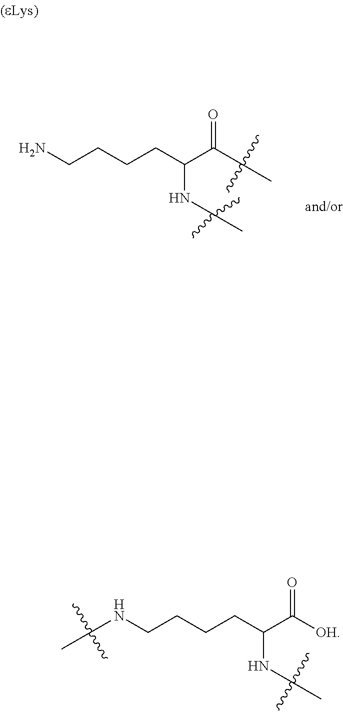 [SMILES: *NC(CCCCN)C(*)=O ; *NCCCCC(N*)C(=O)O]

In some embodiments, the amino acid residue in L^{A}, L^{E} and/or L^{Z} is derived from a β- amino acid or a γ-amino acid. In some embodiments, the β- amino acid is β- alanine.

In some embodiments, the amino acid residue in L^{A}, L^{E} and/or L^{Z} is derived from an unnaturally occurring amino acid. In some embodiments, the unnaturally occurring amino acid comprises a hydrophobic side chain. In some embodiments, the unnatural amino acid is derived from any one selected from the group consisting of noreleucine (Nle), norvaline (Nva), 2-aminooctanoic acid (Aoc), 2-naphthylalanine (2-Nal), 3-(trifluoromethyl)phenylalanine (TFP), homophenylalanine (hPhe), cyclohexylalanine (Cha), 1-naphthylalanine (1-Nal), 4-benzoyl-L-phenylalanine (Bpa), 2-methoxy-4-vinylphenylalanine (MvF), 4-fluorophenylalanine (4-F-Phe), 4-phenyl-2,3-dihydroxy-6-nitrophenylalanine (pNIPA), 2-(2-naphthyl)alanine (2-Nal-ala), 4-(4-propoxyphenyl)alanine (Ppa), 4-carboxyphenylalanine (4-CPA), 4-butylphenylalanine (Bua), 2-nitrophenylalanine (2-Npa), 4-azidophenylalanine (4-AzF), 2-(4-nitrophenyl)ethylalanine (2-Npe), 3-iodo-L-tyrosine (Ity), or 5,5,5-trifluoroleucine (TFL).

In some embodiments, when L^{Z} is a non-cleavable linker and/or one of L^{A} and L^{E} is a non-cleavable linker, each non-cleavable linker independently comprises one or more groups selected from the group consisting of one or more amino acids residues derived from Ala, Arg, Asn, Asp, Cys, Gln, Glu, Gly, His, Ile, Leu, Lys, Met, Phe, Pro, Ser, Thr, Trp, Tyr, Val, Pyl, Sec or PCL or the D enantiomers thereof; one or more amino acids residues derived from DAB or DAP; R¹NC₁₋₂₀alkyleneNR²; C(O)C₁₋₂₀alkyleneC(O); R¹NC₁₋₂₀alkyleneC(O); and C(O)C₁₋₂₀alkyleneNR², the latter 4 groups being optionally interrupted by one or more of S, O, C(O)NH, NHC(O), C₄₋₆cycloalkyl and C₄₋₆heterocycloalkyl wherein each R¹ and R² is independently selected from the group consisting of H and C₁₋₂alkyl.

In some embodiments, when L^{Z} is a non-cleavable linker and/or one of L^{A} and L^{E} is a non-cleavable linker, each non-cleavable linker independently comprises one or more groups selected from the group consisting of one or more amino acids residues derived from Ala, Arg, Asn, Asp, Cys, Gln, Glu, Gly, His, Ile, Leu, Lys, Met, Phe, Pro, Ser, Thr, Trp, Tyr, Val, Pyl, Sec of PCL or the D enantiomers thereof; one or more amino acids residues derived from DAB or DA,

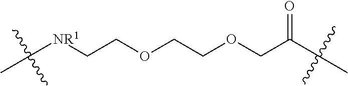

(OEG-R¹), —(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), —(C₂alkyleneO)₆C₂alkyleneC(O) (PEG6), —(C₂alkyleneO)₆C₂alkyleneC(O) (PEG8), and —(C₂alkyleneO)₆C₂alkyleneC(O) (PEG12), R¹NC₁₋₂₀alkyleneNR², C(O)C₁₋₂₀alkyleneC(O), R¹NC₁₋₂₀alkyleneC(O), and C(O)C₁₋₂₀alkyleneNR², wherein each R¹ and R² is independently selected from the group consisting of H and C₁₋₂alkyl.

In some embodiments, when L^{Z} is a non-cleavable linker and/or one of L^{A} and L^{E} is a non-cleavable linker, each non-cleavable linker independently comprises one or more groups selected from the group consisting of one or more amino acids residues derived from Ala, Arg, Asn, Asp, Cys, Gln, Glu, Gly, His, Ile, Leu, Lys, Met, Phe, Pro, Ser, Thr, Trp, Tyr, Val, Pyl, Sec, and PCL, and the D enantiomers thereof;

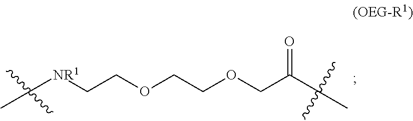

C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), —(C₂alkyleneO)₆C₂alkyleneC(O) (PEG6), —(C₂alkyleneO)₆C₂alkyleneC(O) (PEG8), and —(C₂alkyleneO)₆C₂alkyleneC(O)

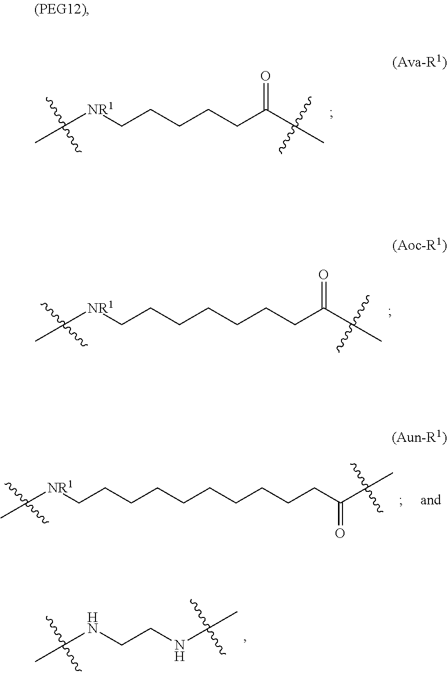

wherein each R¹ is independently selected from the group consisting of H and C₁₋₂alkyl.

In some embodiments, L^{Z} comprises one or more groups selected from the group consisting of

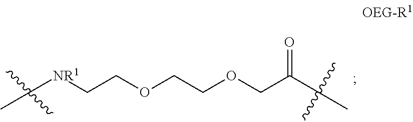

—(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), —(C₂alkyleneO)₆C₂alkyleneC(O) (PEG6), —(C₂alkyleneO)C₂alkyleneC(O) (PEG8), and —(C₂alkyleneO)₆C₂alkyleneC(O) (PEG12), R¹NC₁₋₂₀alkyleneNR², C(O)C₁₋₂₀alkyleneC(O), R¹NC₁₋₂₀alkyleneC(O), and C(O)C₁₋₂₀alkyleneNR², wherein each R¹ and R² is independently selected from the group consisting of H and C₁₋₂alkyl.

In some embodiments, L^{Z} comprises one or more groups selected from the group consisting of

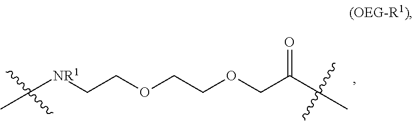

—(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), —(C₂alkyleneO)₆C₂alkyleneC(O) (PEG6), —(C₂alkyleneO)₆C₂alkyleneC(O) (PEG8), and -(C₂alkyleneO)₆C₂alkyleneC(O)

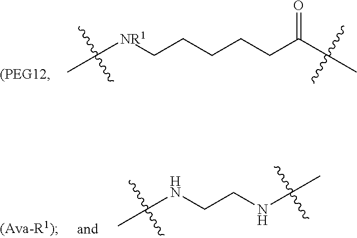

wherein each R¹ and R² is independently selected from the group consisting of H and C₁₋₂alkyl.

Therefore, in some embodiments, L^{Z} is a non-cleavable linker comprising one or more groups selected from the group consisting of OEG-R¹,—(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), —(C₂alkyleneO)₆C₂alkyleneC(O) (PEG6), —(C₂alkyleneO)₆C₂alkyleneC(O) (PEG8), and —(C₂alkyleneO)₆C₂alkyleneC(O) (PEG12), R¹NC₁₋₂₀alkyleneNR², C(O)C₁₋₂₀alkyleneC(O), R¹NC₁₋₂₀alkyleneC(O), and C(O)C₁₋₂₀alkyleneNR², wherein each R¹ and R² is independently selected from the group consisting of H and C₁₋₂alkyl.

In some embodiments, L^{Z} is a non-cleavable linker comprising one or more groups selected from the group consisting of

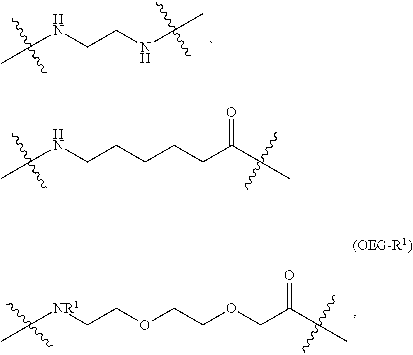

wherein each R¹ and R² is independently selected from the group consisting of H and C₁₋₂alkyl.

In some embodiments, one of L^{A} and L^{E} is a non-cleavable linker, and the non-cleavable linker comprises one or more groups selected from the group consisting of one or more amino acids residues derived from Ala, Arg, Asn, Asp, Cys, Gln, Glu, Gly, His, Ile, Leu, Lys, Met, Phe, Pro, Ser, Thr, Trp, Tyr, Val, Pyl, Sec or PCL or the D enantiomers thereof; one or more amino acids residues derived from DAB or DAP; OEG-R¹, —(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), —(C₂alkyleneO)₆C₂alkyleneC(O) (PEG6), —(C₂alkyleneO)₆C₂alkyleneC(O) (PEG8), —(C₂alkyleneO)₆C₂alkyleneC(O) (PEG12), R¹NC₁₋₂₀alkyleneNR², C(O)C₁₋₂₀alkyleneC(O), R¹NC₁₋₂₀alkyleneC(O), and C(O)C₁₋₂₀alkyleneNR², wherein each R¹ and R² is independently selected from the group consisting of H and C₁₋₂alkyl.

In some embodiments, one of L^{A} and L^{E} is a non-cleavable linker, and the non-cleavable linker comprises one or more groups selected from the group consisting of one or more amino acids residues derived from Ala, Arg, Asn, Asp, Cys, Gln, Glu, Gly, His, Ile, Leu, Lys, Met, Phe, Pro, Ser, Thr, Trp, Tyr, Val, Pyl, Sec, PCL, or the D enantiomers thereof; OEG-R¹; —(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), a —(C₂alkyleneO)₆C₂alkyleneC(O) (PEG6), —(C₂alkyleneO)₆C₂alkyleneC(O) (PEG8); —(C₂alkyleneO)₆C₂alkyleneC(O) (PEG12); Ava-R¹; Aoc-R¹; Aun-R¹ or

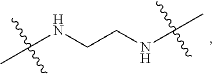

wherein each R¹ is independently selected from the group consisting of H and C₁₋₂alkyl.

In some embodiments, one of L^{A} and L^{E} of the conjugate is a non-cleavable linker, and the non-cleavable linker comprises one or more groups selected from the group consisting of one or more amino acids residues derived from Glu, Gly, Lys, Phe, Tyr, and Val, or the D enantiomers thereof; an amino acid residue derived from DAB, R¹NC₁₋₂₀alkyleneNR², C(O)C₁₋₂₀alkyleneC(O), R¹NC₁₋₂₀alkyleneC(O), and C(O)C₁₋₂₀alkyleneNR², wherein each R¹ and R² is independently selected from the group consisting of H and C₁₋₂alkyl.

In some embodiments, one of L^{A} and L^{E} of the conjugate is a non-cleavable linker, and the non-cleavable linker comprises one or more groups selected from the group consisting of one or more amino acids residues derived from Glu, Gly, Lys, Phe, Tyr, and Val, and the D enantiomers thereof; OEG-R¹; —(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), a —(C₂alkyleneO)₆C₂alkyleneC(O) (PEG6), —(C₂alkyleneO)₆C₂alkyleneC(O) (PEG8); Ava-R¹; Aoc-R¹; Aun-R¹ or

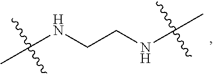

wherein each R¹ is independently selected from the group consisting of H and C₁₋₂alkyl.

In some embodiments, L^{A} is a non-cleavable linker, and the non-cleavable linker comprises one or more groups selected from the group consisting of amino acids residues derived from Gly, Glu, OEG-R¹, —(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), a —(C₂alkyleneO)₆C₂alkyleneC(O) (PEG6), —(C₂alkyleneO)₆C₂alkyleneC(O) (PEG8); R¹NC₁₋₂₀alkyleneNR², C(O)C₁₋₂₀alkyleneC(O), R¹NC₁₋₂₀alkyleneC(O), and C(O)C₁₋₂₀alkyleneNR², wherein each R¹ and R² is independently selected from the group consisting of H and C₁₋₂alkyl.

In some embodiments, L^{A} is a non-cleavable linker, and the non-cleavable linker comprises one or more groups selected from the group consisting of amino acids residues derived from Glu, OEG-R^{1′}—(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), a —(C₂alkyleneO)₆C₂alkyleneC(O) (PEG6), —(C₂alkyleneO)₆C₂alkyleneC(O) (PEG8) and

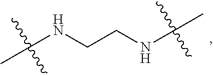

wherein R¹ is selected from the group consisting of H and C₁₋₂alkyl. In some embodiments, L^{A} is a non-cleavable linker, and the non-cleavable linker comprises one or more groups selected from the group consisting of amino acids residues derived from Glu and OEG-R¹ wherein R¹ is selected from the group consisting of H and C₁₋₂alkyl.

In some embodiments, the amino acid residue derived from Glu is γGlu

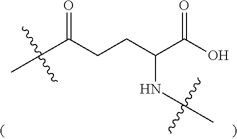 [SMILES: *NC(CCC(*)=O)C(=O)O]

and L^{A} is a non-cleavable linker, and the non-cleavable linker comprises one or more groups selected from the group consisting of γGlu

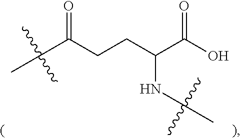 [SMILES: *NC(CCC(*)=O)C(=O)O]

OEG-R¹, —(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), a —(C₂alkyleneO)₆C₂alkyleneC(O) (PEG6), —(C₂alkyleneO)₆C₂alkyleneC(O) (PEG8); wherein R¹ is selected from the group consisting of H and C₁₋₂alkyl. In some embodiments, L^{A} is a non-cleavable linker, and the non-cleavable linker comprises one or more of γGlu

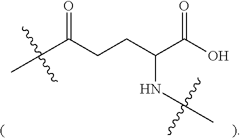 [SMILES: *NC(CCC(*)=O)C(=O)O]

In some embodiments, L^{A} is a non-cleavable linker, and the non-cleavable linker comprises one or more of OEG-R¹ wherein R¹ is selected from the group consisting of H and C₁₋₂alkyl. In some embodiments, the non-cleavable linker is or comprises γGlu-(OEG)₁₋₃. In some embodiments, the non-cleavable linker is or comprises γGlu-(OEG-R¹) wherein R¹ is selected from the group consisting of H and C₁₋₂alkyl.

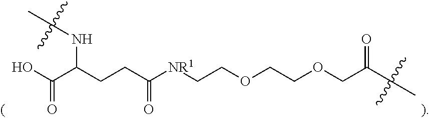

In some embodiments, L^{A} comprises two or more of OEG-R¹ wherein R¹ is selected from the group consisting of H and C₁₋₂alkyl.

In some embodiments, L^{E} is a non-cleavable linker, and the non-cleavable linker comprises one or more groups selected from the group consisting of OEG-R¹, —(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), a —(C₂alkyleneO)₆C₂alkyleneC(O) (PEG6), —(C₂alkyleneO)₆C₂alkyleneC(O) (PEG8); R¹NC₁₋₂₀alkyleneNR², C(O)C₁₋₂₀alkyleneC(O), R¹NC₁₋₂₀alkyleneC(O), and C(O)C₁₋₂₀alkyleneNR², wherein each R¹ and R² is independently selected from the group consisting of H and C₁₋₂alkyl. In some embodiments, L^{E} is a non-cleavable linker, and the non-cleavable linker comprises one or more groups selected from the group consisting of OEG-R¹ —(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), a —(C₂alkyleneO)₆C₂alkyleneC(O) (PEG6), —(C₂alkyleneO)₆C₂alkyleneC(O) (PEG8) and

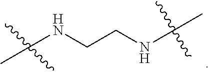

wherein R¹ is selected from the group consisting of H and C₁₋₂alkyl. In some embodiments, L^{E} comprises two or more of OEG-R¹ wherein R¹ is selected from the group consisting of H and C₁₋₂alkyl.

In some embodiments, one or both of L^{A} and L^{E} is a cleavable linker, and each cleavable linker independently comprises one or more groups selected from the group consisting of R⁵NC₁₋₂₀alkyleneC(O); C(O)C₁₋₂₀alkyleneNR⁶; C(O)C₁₋₂₀alkyleneO and OC₁₋₂₀alkyleneC(O), each of which is independently and optionally interrupted by one or more of S—S, C(O)O, OC(O), O, C(O)NH and NHC(O), and wherein each alkyl and alkylene is optionally substituted with one or more substituents selected from the group consisting of halo, COOH, NR⁷R⁷, and C₁₋₄alkyleneNR⁷R⁸;
wherein R⁵, R⁶, R⁷ and R⁸ are independently selected from the group consisting of H and C₁₋₄alkyl.

In some embodiments, one or both of L^{A} and L^{E} is a cleavable linker, and the cleavable linker comprises R⁵NC₁₋₂₀alkyleneC(O), each of which is independently and optionally interrupted by one or more of S—S, C(O)O, OC(O), O, C(O)NH and NHC(O), provided R⁵NC₁₋₂₀alkyleneC(O) is interrupted by at least one of S—S, C(O)O and OC(O); or the cleavable linker comprises at least one group connected to C(O)C₁₋₂₀alkyleneO or OC₁₋₂O alkyleneC(O) to form a cleavable moiety selected from the group consisting of C(O)O and OC(O). In some embodiments, one or both of L^{A} and L^{E} is a cleavable linker, and the cleavable linker comprises R⁵NC₁₋₂₀alkyleneC(O) optionally interrupted by one or more of S—S, C(O)O, OC(O), O, C(O)NH and NHC(O), provided R⁵NC₁₋₂₀alkyleneC(O) is interrupted by at least one of S—S, C(O)O ad OC(O); or the cleavable linker comprises at least one amino acid residue connected to C(O)C₁₋₂₀alkyleneO or OC₁₋₂₀alkyleneC(O) to form a cleavable moiety selected from the group consisting of C(O)O and OC(O). In some embodiments, one or both of L^{A} and L^{E} is a cleavable linker, and the cleavable linker comprises R⁵NC₁₋₂₀alkyleneC(O) optionally interrupted by one to five of S—S, C(O)O, OC(O), O, C(O)NH and NHC(O), provided R⁵NC₁₋₂₀alkyleneC(O) is interrupted by at least one of S—S, C(O)O ad OC(O); or the cleavable linker comprises at least one amino acid residue connected to C(O)C₁₋₂₀alkyleneO or OC₁₋₂₀alkyleneC(O) to form a cleavable moiety selected from the group consisting of C(O)O and OC(O).

In some embodiments, one or both of L^{A} and L^{E} of the conjugate is a cleavable linker, and the cleavable linker comprises R⁵NC₁₋₂₀alkyleneC(O) optionally interrupted by one to five of S—S, C(O)O, OC(O), O, C(O)NH and NHC(O), provided R⁵NC₁₋₂₀alkyleneC(O) is interrupted by at least one of S—S, C(O)O and OC(O).

In some embodiments, one or both of L^{A} and L^{E} is a cleavable linker, and the cleavable linker comprises at least one group selected from the group consisting of

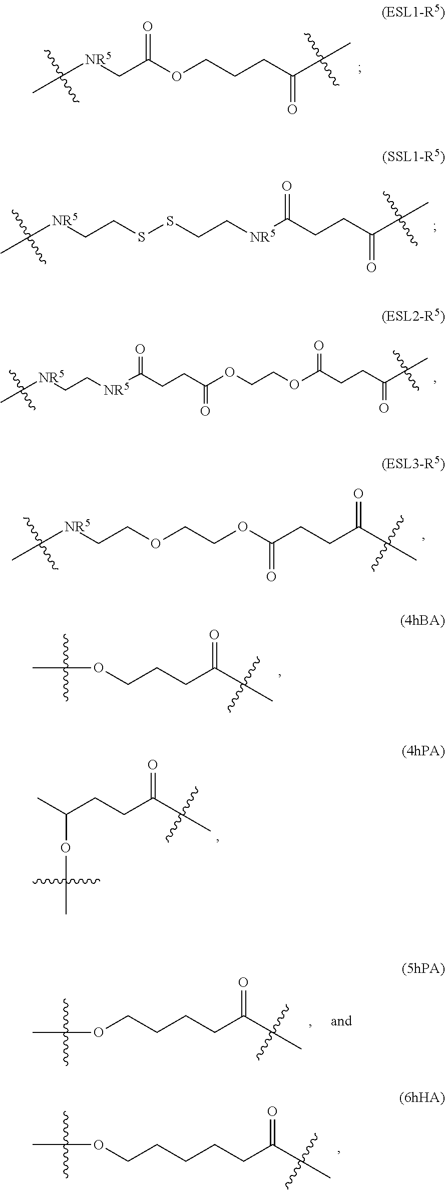

wherein each R⁵ is selected from the group consisting of H and C₁₋₄alkyl.

In some embodiments, one or both of L^{A} and L^{E} of the conjugate is a cleavable linker, and the cleavable linker comprises at least one amino acid residue connected to C(O)C₁₋₂₀alkyleneO or OC₁₋₂₀alkyleneC(O) to form a cleavable moiety selected from the group consisting of C(O)O and OC(O). In some embodiments, one or both of L^{A} and L^{E} is a cleavable linker, and the cleavable linker comprises at least one amino acid residue connected to C(O)C₁₋₁₀alkyleneO or OC₁₋₁₀alkyleneC(O) to form a cleavable moiety selected from the group consisting of C(O)O and OC(O). In some embodiments, the at least one amino acid residue connected to C(O)C₁₋₁₀alkyleneO or OC₁₋₁₀alkyleneC(O) is derived from an amino acid selected from the group consisting of a naturally occurring amino acid or the D-enantiomer thereof, DAB and DAP. In some embodiments, amino acid is selected from the group consisting of a Gly, Leu, DAB and DAP. Therefore, in some embodiments, one or both of L^{A} and L^{E} is a cleavable linker, and the cleavable linker comprises at least one amino acid residue a naturally occurring amino acid or the D-enantiomer thereof, DAB and DAP connected to C(O)C₁₋₁₀alkyleneO or OC₁₋₁₀alkyleneC(O) to form a cleavable moiety selected from the group consisting of C(O)O and OC(O).

In some embodiments, one or both of L^{A} and L^{E} of the conjugate is a cleavable linker, and the cleavable linker comprises enzymatically cleavable peptide sequences. In some embodiments, the enzymatically cleavable peptide sequences are peptide sequences cleavable by the kidney brush border enzyme, thermolysin, prolineendopeptidase, fibroblast activation protein (FAP), neprilysin or a general endopeptidase. In some embodiments, the peptide sequence cleavable by the kidney brush border enzyme is Met-Val-Lys. In some embodiments, the peptide sequence cleavable by thermolysin is Ala-Val. In some embodiments, the peptide sequence cleavable by prolineendopeptidase is Ala-Pro or Gly-Pro. In some embodiments, the peptide sequence cleavable by FAP is Gly-Pro. In some embodiments, the peptide sequence cleavable by neprilysin is Ser-Ser, Ser-Gly, Gly-Ala or Ser-Lys. Therefore, in some embodiments, the enzymatically cleavable peptide sequence is selected from the group consisting of Met-Val-Lys, Ala-Val, Ala-Pro, Gly-Pro, Ser-Ser, Ser-Gly, Gly-Ala and Ser-Lys.

In some embodiments, the cleavable linker optionally further comprises one or more groups selected from the group consisting of amino acid residues derived from Ala, Arg, Asn, Asp, Cys, Gln, Glu, Gly, His, Ile, Leu, Lys, Met, Phe, Pro, Ser, Thr, Trp, Tyr, Val, Pyl, Sec or PCL or the D enantiomers thereof; one or more amino acids residues derived from DAB or DAP; R⁵NC₁₋₂₀alkyleneNR⁶; and R⁵NC₁₋₂₀alkyleneC(O) optionally interrupted by one or more of O. In some embodiments, the cleavable linker optionally further comprises one or more groups selected from the group consisting of amino acid residues derived from Ala, Arg, Asn, Asp, Cys, Gln, Glu, Gly, His, Ile, Leu, Lys, Met, Phe, Pro, Ser, Thr, Trp, Tyr, Val, Pyl, Sec, and PCL and the D enantiomers thereof, OEG-R¹; Ava-R¹; Aoc-R¹; Aun-R¹ and

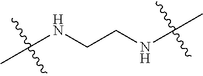

wherein each R¹ is independently selected from the group consisting of H and C₁₋₂alkyl.

In some embodiments, one or both L^{A} and L^{E} is a cleavable linker, and each cleavable linker independently comprises one or more groups selected from the group consisting of one or more amino acid residues derived from Gly, Glu, Leu, Phe, Tyr, or Lys, or the D enantiomers thereof; one or more amino acid residues derived from DAP or DAB; R⁵NC₁₋₂₀alkyleneNR⁶; and R⁵NC₁₋₂₀alkyleneC(O) optionally interrupted by one or more of O; and

at least one R⁵NC₁₋₂₀alkyleneC(O) optionally interrupted by one or more of S—S, C(O)O, OC(O), O, C(O)NH and NHC(O), provided R⁵NC₁₋₂₀alkyleneC(O) is interrupted by at least one of S—S, C(O)O and OC(O), or at least one amino acid residue connected to C(O)C₁₋₁₀alkyleneO or OC₁₋₁₀alkyleneC(O) to form a cleavable moiety selected from the group consisting of C(O)O and OC(O), or an enzymatically cleavable peptide sequence selected from the group consisting of Met-Val-Lys, Ala-Val, Ala-Pro, Gly-Pro, Ser-Ser, Ser-Gly, Gly-Ala and Ser-Lys. In some embodiments, one or both L^{A} and L^{E} is a cleavable linker, and each cleavable linker independently comprises one or more groups selected from the group consisting of amino acid residues derived from Gly, Glu, Leu, Phe, Tyr, or Lys, or the D enantiomers thereof; one or more amino acid residues derived from DAP or DAB; R⁵NC₁₋₂₀alkyleneNR⁶; and R⁵NC₁₋₂₀alkyleneC(O) optionally interrupted by one or more of O; and

at least one amino acid residue connected to C(O)C₁₋₁₀alkyleneO or OC₁₋₁₀alkyleneC(O) to form a cleavable moiety selected from the group consisting of C(O)O and OC(O). In some embodiments, one or both L^{A} and L^{E} is a cleavable linker, and each cleavable linker independently comprises one or more groups selected from the group consisting of amino acid residues derived from Gly, Glu, Leu, Phe, Tyr, or Lys, or the D enantiomers thereof; one or more amino acid residues derived from DAP or DAB; R⁵NC₁₋₂₀alkyleneNR⁶; and R⁵NC₁₋₂₀alkyleneC(O) optionally interrupted by one or more of O; and

at least one group R⁵NC₁₋₂₀alkyleneC(O) optionally interrupted by one or more of S—S, C(O)O, OC(O), O, C(O)NH and NHC(O), provided R⁵NC₁₋₂₀alkyleneC(O) is interrupted by at least one of S—S, C(O)O ad OC(O), or

an enzymatically cleavable peptide sequence selected from the group consisting of Met-Val-Lys, Ala-Val, Ala-Pro, Gly-Pro, Ser-Ser, Ser-Gly, Gly-Ala and Ser-Lys. In some embodiments, one or both L^{A} and L^{E} is a cleavable linker, and each cleavable linker independently comprises one or more groups selected from the group consisting of amino acid residues derived from Glu, Lys, Phe, Tyr, and the D enantiomers thereof;

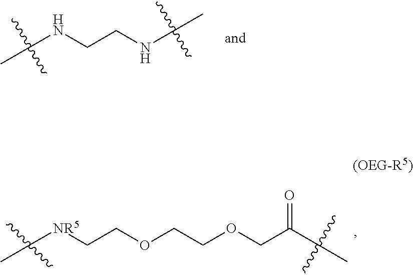

—(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), —(C₂alkyleneO)₆C₂alkyleneC(O) (PEG6), —(C₂alkyleneO)₆C₂alkyleneC(O) (PEG8), and —(C₂alkyleneO)₆C₂alkyleneC(O) (PEG12), wherein each R⁵ is selected from the group consisting of H and C₁₋₄alkyl; and

at least one group selected from the group consisting of SSL1-R⁵, ESL2-R⁵ and ESL3-R⁵ wherein each R⁵ is selected from the group consisting of H and C₁₋₄alkyl or an enzymatically cleavable peptide sequence selected from the group consisting of Met-Val-Lys, Ala-Val, Ala-Pro, Gly-Pro, Ser-Ser, Ser-Gly, Gly-Ala and Ser-Lys. In some embodiments, one or both L^{A} and L^{E} is a cleavable linker, and each cleavable linker independently comprises one or more groups selected from the group consisting of amino acid residues derived from Gly, Glu, Leu, Phe, Tyr, or Lys, or the D enantiomers thereof; one or more amino acid residues derived from DAP and DAB; R⁵NC₁₋₂₀alkyleneNR⁶; and R⁵NC₁₋₂₀alkyleneC(O) optionally interrupted by one or more of O; and

at least one group R⁵NC₁₋₂₀alkyleneC(O) optionally interrupted by one or more of S—S, C(O)O, OC(O), O, C(O)NH and NHC(O), provided R⁵NC₁₋₂₀alkyleneC(O) is interrupted by at least one of S—S, C(O)O ad OC(O).

In some embodiments, L^{A} is a cleavable linker, and the cleavable linker comprises one or more groups selected from the group consisting of amino acid residues derived from Gly, Glu, Leu, Phe, Tyr, or Lys, or the D enantiomers thereof; one or more amino acid residues derived from DAP or DAB; R⁵NC₁₋₂₀alkyleneNR⁶; and R⁵NC₁₋₂₀alkyleneC(O) optionally interrupted by one or more of O; and

at least one R⁵NC₁₋₂₀alkyleneC(O) optionally interrupted by one or more of S—S, C(O)O, OC(O), O, C(O)NH and NHC(O), provided R⁵NC₁₋₂₀alkyleneC(O) is interrupted by at least one of S—S, C(O)O and OC(O),

at least one amino acid residue connected to C(O)C₁₋₁₀alkyleneO or OC₁₋₁₀alkyleneC(O) to form a cleavable moiety selected from the group consisting of C(O)O and OC(O), or an enzymatically cleavable peptide sequence selected from the group consisting of Met-Val-Lys, Ala-Val, Ala-Pro, Gly-Pro, Ser-Ser, Ser-Gly, Gly-Ala and Ser-Lys. In some embodiments, L^{A} is a cleavable linker, and the cleavable linker comprises one or more groups selected from the group consisting of one or more amino acid residues derived from Gly, Glu, Leu, Phe, Tyr, or Lys, or the D enantiomers thereof; one or more amino acid residues derived from DAP and DAB; R⁵NC₁₋₂₀alkyleneNR⁶; and R⁵NC₁₋₂₀alkyleneC(O) optionally interrupted by one or more of O; and

at least one amino acid residue connected to C(O)C₁₋₁₀alkyleneO or OC₁₋₁₀alkyleneC(O) to form a cleavable moiety selected from the group consisting of C(O)O and OC(O). In some embodiments, L^{A} is a cleavable linker, and the cleavable linker comprises one or more groups selected from the group consisting of amino acid residues derived from Glu, Lys, Phe, Tyr, and the D enantiomers thereof;

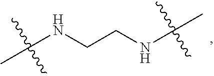 [SMILES: *NCCN*]

OEG-R⁵, —(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), —(C₂alkyleneO)₆C₂alkyleneC(O) (PEG6), —(C₂alkyleneO)₆C₂alkyleneC(O) (PEG8), and —(C₂alkyleneO)₆C₂alkyleneC(O) (PEG12), wherein each R⁵ is selected from the group consisting of H and C₁₋₄alkyl.; and at least one group selected from the group consisting of SSL1-R⁵, ESL2-R⁵ and ESL3-R⁵ wherein each R⁵ is selected from the group consisting of H and C₁₋₄alkyl or an enzymatically cleavable peptide sequence selected from the group consisting of Met-Val-Lys, Ala-Val, Ala-Pro, Gly-Pro, Ser-Ser, Ser-Gly, Gly-Ala and Ser-Lys.

In some embodiments, L^{E} is a cleavable linker, and the cleavable linker comprises one or more groups selected from the group consisting of one or more amino acid residues derived from Gly, Glu, Leu, Phe, Tyr, or Lys, or the D enantiomers thereof; one or more amino acid residues derived from DAP or DAB; R⁵NC₁₋₂₀alkyleneNR⁶; and R⁵NC₁₋₂₀alkyleneC(O) optionally interrupted by one or more of O; and

at least one R⁵NC₁₋₂₀alkyleneC(O) optionally interrupted by one or more of S—S, C(O)O, OC(O), O, C(O)NH and NHC(O), provided R⁵NC₁₋₂₀alkyleneC(O) is interrupted by at least one of S—S, C(O)O ad OC(O), or

at least one amino acid residue connected to C(O)C₁₋₁₀alkyleneO or OC₁₋₁₀alkyleneC(O) to form a cleavable moiety selected from the group consisting of C(O)O and OC(O), or an enzymatically cleavable peptide sequence selected from the group consisting of Met-Val-Ala-Val, Ala-Pro, Gly-Pro, Ser-Ser, Ser-Gly, Gly-Ala and Ser-Lys. In some embodiments, L^{E} is a cleavable linker, and the cleavable linker comprises one or more groups selected from the group consisting of one or more amino acid residues derived from Gly, Glu, Leu, Phe, Tyr, or Lys, or the D enantiomers thereof; one or more amino acid residues derived from DAP and DAB; R⁵NC₁₋₂₀alkyleneNR⁶; and R⁵NC₁₋₂₀alkyleneC(O) optionally interrupted by one or more of O; and at least one amino acid residue connected to C(O)C₁₋₁₀alkyleneO or OC₁₋₁₀alkyleneC(O) to form a cleavable moiety selected from the group consisting of C(O)O and OC(O). In some embodiments, L^{E} is a cleavable linker, and the cleavable linker comprises one or more of OEG-R⁵, —(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), —(C₂alkyleneO)₆C₂alkyleneC(O) (PEG6), —(C₂alkyleneO)₆C₂alkyleneC(O) (PEG8), and —(C₂alkyleneO)₆C₂alkyleneC(O) (PEG12); and at least one group selected from the group consisting of ESL1-R5, SSL1-R5, ESL2-R5, ESL3-R5, ESL1, ESL2, ESL3, 4hPA-Gly, 5hPA-Gly, 4hBA-Leu, 6hHA-Gly, Gly-5aPOH, I-PADT, and SA-5aPOH, wherein each R⁵ is selected from the group consisting of H and C₁₋₄alkyl, and an enzymatically cleavable peptide sequence selected from the group consisting of Met-Val-Lys, Ala-Val, Ala-Pro, Gly-Pro, Ser-Ser, Ser-Gly, Gly-Ala and Ser-Lys. In an exemplary embodiment, L^{E} is a cleavable linker, and the cleavable linker is or comprises

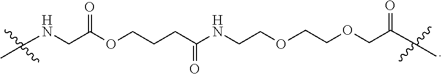 [SMILES: *NCC(=O)OCCCC(=O)NCCOCCOCC(*)=O]

In some embodiments, R⁵, R⁶, R⁷ and R⁸ are independently selected from the group consisting of H, CH₃, CH₂CH₃, CH₂CH₂CH₃, CH(CH₃)₂, and C(CH₃)₃. In some embodiments, R⁵, R⁶, R⁷ and R⁸ are independently selected from the group consisting of H and C₁₋₃alkyl. In some embodiments, R⁵, R⁶, R⁷ and R⁸ are independently selected H, CH₃, CH₂CH₃, and CH(CH₃)₂. In some embodiments, R⁵, R⁶, R⁷ and R⁸ are independently selected H, and CH₃.

In some embodiments, L^{A}, L^{E}, and L^{Z} each independently comprise 1 to 15 groups, 1 to 12 groups, 1 to 10 groups, 1 to 8 groups, or 1 to 6 groups that are connected together.

In some embodiments, L^{A}, L^{E}, and L^{Z} each independently comprise 1 to 15 groups, 1 to 12 groups or 1 to 10 groups connected together. In some embodiments, L^{A}, L^{E}, and L^{Z} each independently consist of, 1 to 5 groups, 1 to 4 groups, 1 to 3 groups, or 1 or 2 groups connected together. In some embodiments, L^{A}, L^{E}, and L^{Z} each independently consist of 1 to 4 groups, 1 to 3 groups, or 1 or 2 groups connected together.

In some embodiments, L^{A} is

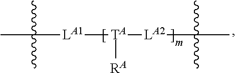

### L^{Z} is

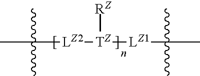

### and L^{E} is

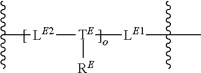

and the conjugate of Formula I is a conjugate of Formula I-D or a pharmaceutically acceptable salt and/or solvate thereof

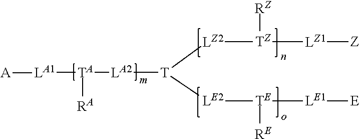

wherein
A, Z, E and T are as defined in Formula I
T^{A}, T^{Z}, T^{E} are each independently T as defined in formula I and T^{A}, T^{Z}, T^{E} and T are the same or different;
L^{A1} and L^{A2} are each independently L^{A} as defined in formula I;
L^{E1} and L^{E2} are each independently L^{E} as defined in formula I;
L^{Z1} and L^{Z2} are each independently L^{Z} as defined in formula I;
R^{A} is selected from the group consisting of

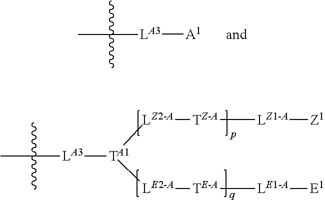

R^{Z} is selected from the group consisting of

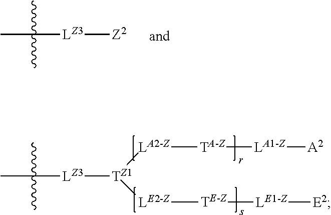

R^{E} is selected from the group consisting of

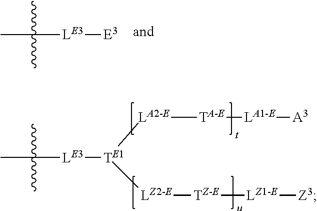

T^{A1}, T^{Z-A}, T^{E-A}, T^{Z1}, T^{A-Z}, T^{E-Z}, T^{E1}, T^{Z-E}, T^{A-E} are each independently T as defined in Formula I and T, T^{A1}, T^{Z-A}, T^{E-A}, T^{Z1}, T^{A-Z}, T^{E-Z}, T^{E1}, T^{Z-E} and T^{A-E} are the same or different;
L^{A3}, L^{A1-Z}, L^{A2-Z}, L^{A1-E} and L^{A2-E} are each independently L^{A} as defined in Formula I;
L^{E1-A}, L^{E2-A}, L^{E1-Z}, L^{E2-Z} and L^{E3} are each independently L^{E} as defined in Formula I;
L^{Z3}, L^{Z1-A}, L^{Z2-A}, L^{Z1-E} and L^{Z2-E} are each independently L^{Z} as defined in Formula I;
A¹, A² and A³ are each independently A as defined in Formula I and A¹, A², A³ and A are the same or different;
Z¹, Z² and Z³ each independently Z as defined in Formula I and Z¹, Z², Z³ and Z are the same or different;
E¹, E² and E³ each independently E as defined in Formula I and E¹, E², E³ and E are the same or different; and
m, n, o, p, q, r, s, t and u are each independently selected from the group consisting of 0 and 1,
wherein at least one of L^{A3}, L^{E1-A}, L^{E2-A}, L^{A1-Z}, L^{A2-Z}, L^{E1-Z}, L^{E2-Z}, L^{E3}, L^{A1-E} and L^{A2-E} is a cleavable linker.

In some embodiments, T is a branching group that is at least trivalent comprising at least a first terminal functionality, a second terminal functionality and a third terminal functionality, which are the same or different and bind to L^{A} (or alternatively T^{A}, L^{A1} or A), L^{Z2} (or alternatively T^{Z}, L^{Z1} or Z) and L^{E2} (or alternatively T^{E}, L^{E1} or E), respectively. In an exemplary embodiment, T is lysine or DAB. In an exemplary embodiment, T is lysine. Therefore, in exemplary embodiments, the conjugate of Formula I-D is a conjugate of Formula I-E or I-F.

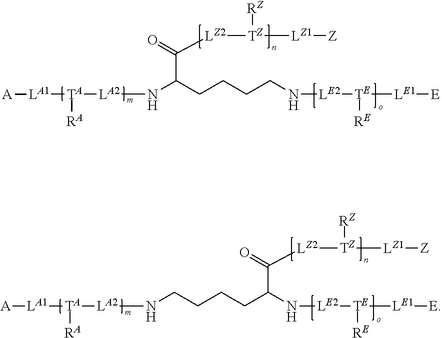

In some embodiments, when p and r are each 0, then L^{A1} is L^{A} as defined in Formula I, L^{Z1} is L^{Z} as defined in Formula I, and L^{E1} is L^{E} as defined in Formula I and the conjugate of Formula I-D is the same as the conjugate of Formula I.

In some embodiments, T^{A}, T^{Z} and T^{E} are each independently a branching group that is at least trivalent comprising at least a first terminal functionality, a second terminal functionality and a third terminal functionality, which are the same or different and bind to L^{A1}, L^{A2} and R^{A} (or alternatively A and T), to L^{Z1}, L^{Z2} and R^{Z} (or alternatively Z and T), and to L^{E1}, L^{E2} and R^{E} (or alternatively E and T), respectively. In some embodiments, T^{A}, T^{Z} and T^{E} are each independently a branching group that is at least trivalent comprising at least a first terminal functionality, a second terminal functionality and a third terminal functionality, which are the same or different and bind to L^{A1}, L^{A2} and R^{A} (or alternatively A or T), to L^{Z1}, L^{Z2} and R^{Z} (or alternatively Z and T), and to L^{Ed1}, L^{E2} and R^{E} (or alternatively E and T), an amide group, a thiourea groups, a urea or a thioamide group. In some embodiments, when bonded to L^{A1}, L^{A2} and R^{A} (or alternatively A or T) and to L^{Ed1}, L^{E2} and R^{E} (or alternatively E and T), an ester group, a thioester group, a carbonate group, a carbamate group, a disulfide bond, a hydrazone group, or an oxime group such as a ketoxime or aldoxime is further formed. In some embodiments, T^{A}, T^{Z} and T^{E} are each independently selected from the group consisting of an amino acid residue derived from lysine and/or glutamine and a trimesic acid residue,

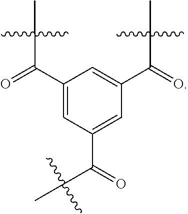 [SMILES: *C(=O)c1cc(C(*)=O)cc(C(*)=O)c1]

In some embodiments, T^{A}, T^{Z} and T^{E} are each independently selected from the group consisting of an amino acid residue derived from lysine and/or glutamine, DAB and TMA. In some embodiments, T^{A}, T^{Z} and T^{E} are each independently selected from the group consisting of an amino acid residue derived from lysine and a trimesic acid residue.

In some embodiments, T is a tetravalent branching group and the compound of Formula I-D (optionally Formula I-E and I-F) further includes a detection moiety (D) and D is conjugated to T via a linker L^{D}, and the conjugate of Formula I-D, I-E and I-F is a conjugate of Formula I(a)-D:

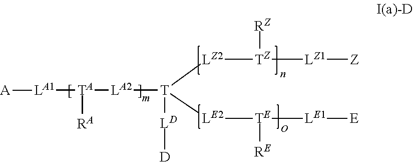

wherein
A, Z, E, T, T^{A}, T^{Z}, T^{E} L^{A1} L^{A2}, L^{E1} L^{E2} L^{Z1}, L^{Z2}, R^{A}, R^{Z}, R^{E}, m, n, and o are as defined in Formula I-D, and
D and L^{D} are as defined in Formula I(a).

In some embodiments, R^{A} is

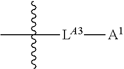

and L^{A}i, L^{A2} and L^{A3} are each independently L^{A} as defined in Formula I. In some embodiments, A¹ and A are the same. In some embodiments, A¹ and A are different.

In some embodiments, R^{Z} is

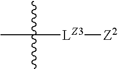

and L^{Z1}, L^{Z2} and L^{Z3} are each independently L^{Z} as defined in Formula I. In some embodiments, Z² and Z are the same. In some embodiments, Z² and Z are different.

In some embodiments, R^{E} is

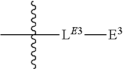

and L^{E1}, L^{E2} and L^{E3} are each independently L^{E} as defined in Formula I. In some embodiments, E³ and E are the same. In some embodiments, E³ and E are different.

In some embodiments, one to three of L^{A3}, L^{E1-A}, L^{E2-A}, L^{A1-Z}, L^{A2-Z}, L^{E1-Z} L^{E2-Z}, L^{E3}, L^{A1-E} and L^{A2-E} are cleavable linkers. In some embodiments, one or two of L^{A3}, L^{E1-A}, L^{E2-A}, L^{A1-Z} L^{A2-Z}, L^{E1-Z} L^{E2-Z}, L^{E3}, L^{A1-E} and L^{A2-E} are cleavable linkers. In some embodiments, each cleavable linker comprises one to three cleavable moieties. In some embodiments, each cleavable linker comprises one or two cleavable moieties.

In some embodiments, m is 1, n and o are both 0, R^{A} is

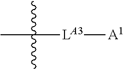

and the conjugate of Formula I-D is a conjugate of Formula I-G

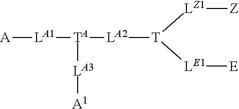

wherein
A, Z, E and T are as defined in Formula I;
L^{A1}, L^{A2} and L^{A3} are each independently L^{A} as defined for in Formula I;
A¹ is A as defined in Formula I and A¹ and A are the same or different;
L^{Z1} is L^{Z} as defined for Formula I;
L^{E1} is L^{E} as defined in Formula I.

In some embodiments, at least one of L^{A1}, L^{A2} L^{A3} and L^{E1} is a cleavable linker.

In some embodiments, T^{A} in the conjugate of Formula I-G is a branching group that is at least trivalent comprising at least a first terminal functionality, a second terminal functionality and a third terminal functionality, which are the same or different and bind to L^{A1}, L^{A2} and R^{A} (or alternatively A and/or T). In exemplary embodiments, T^{A} in the conjugate of Formula I-G is amino acid residue derived from lysine or a trimesic acid residue.

In some embodiments, T in the conjugate of Formula I-G a tetravalent branching group and the compound of Formula I-G further includes a detection moiety (D) and D is conjugated to T via a linker L^{D}, and the conjugate of Formula I-G is a conjugate of Formula I(a)-G:

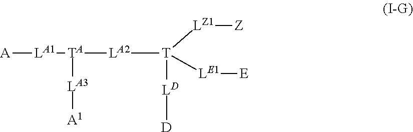

wherein
A¹, A, L^{A1}, L^{A2} L^{A3}, L^{Z1} and L^{E1} as defined in Formula I-D, and
D and L^{D} are as defined in Formula I(a).

In some embodiments, at least one of L^{A1}, L^{A2}, L^{A3} and L^{E1} is a cleavable linker.

In an exemplary embodiment, the A¹ and/or A in the conjugate of Formula I-G or I(a)-G are each independently selected from the group consisting of unsubstituted or substituted C(O)C₁₂₋₁₈alkyleneCOOH.

In an exemplary embodiment, E is selected from the group consisting of DOTA and DOTAGA.

In some embodiments, when one or more of, L^{A1}, L^{A2}, L^{A3} and L^{E1} in the conjugate of Formula I-G or I(a)-G is a non-cleavable linker, each of the non-cleavable linkers independently comprises one or more groups selected from the group consisting of Ala, Arg, Asn, Asp, Cys, Gln, Glu, Gly, His, Ile, Leu, Lys, Met, Phe, Pro, Ser, Thr, Trp, Tyr, Val, Pyl, Sec or PCL or the D enantiomers thereof; one or more amino acids residues derived from DAB or DAP; R¹NC₁₋₂₀alkyleneNR²; C(O)C₁₋₂₀alkyleneC(O); R¹NC₁₋₂₀alkyleneC(O); and C(O)C₁₋₂₀alkyleneNR², the latter 4 groups being optionally interrupted by one or more of S, O, C(O)NH, NHC(O), C₄₋₆cycloalkyl and C₄₋₆heterocycloalkyl wherein each R¹ and R² is independently selected from the group consisting of H and C₁₋₂alkyl.

In some embodiments, when one or more of L^{A1}, L^{A2}, L^{A3} and L^{E1} in the conjugate of Formula I-G is a non-cleavable linker, each non-cleavable linker independently comprises one or more groups selected from the group consisting of one or more amino acids residues derived from Ala, Arg, Asn, Asp, Cys, Gln, Glu, Gly, His, Ile, Leu, Lys, Met, Phe, Pro, Ser, Thr, Trp, Tyr, Val, Pyl, Sec, PCL and the D enantiomers thereof; OEG-R¹; Ava-R¹; Aoc-R¹; Aun-R¹ or

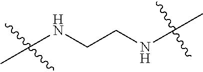

wherein each R¹ is independently selected from the group consisting of H and C₁₋₂alky.

In some embodiments, one or more of L^{A1}, L^{A2}, L^{A3} and L^{E1} in the conjugate of Formula I-G or I(a)-G is independently a cleavable linker and each cleavable linker independently optionally comprises one or more groups selected from the group consisting of one or more amino acid residues derived from Gly, Glu, Leu, Phe, Tyr, or Lys, or the D enantiomers thereof; one or more amino acid residues derived from DAP or DAB; R⁵NC₁₋₂₀alkyleneNR⁶; and R⁵NC₁₋₂₀alkyleneC(O) optionally interrupted by one or more of O; and at least one R⁵NC₁₋₂₀alkyleneC(O) optionally interrupted by one or more of S—S, C(O)O, OC(O), O, C(O)NH and NHC(O), provided R⁵NC₁₋₂₀alkyleneC(O) is interrupted by at least one of S—S, C(O)O and OC(O), or

at least one amino acid residue connected to C(O)C₁₋₁₀alkyleneO or OC₁₋₁₀alkyleneC(O) to form a cleavable moiety selected from the group consisting of C(O)O and OC(O), or an enzymatically cleavable peptide sequence selected from the group consisting of Met-Val-Lys, Ala-Val, Ala-Pro, Gly-Pro, Ser-Ser, Ser-Gly, Gly-Ala and Ser-Lys.

In some embodiments, one or more of L^{A1}, L^{A2}, L^{A3} and L^{E1} in the conjugate of Formula I-G or I(a)-G is independently a cleavable linker and each cleavable linker independently optionally comprises one or more groups selected from the group consisting of amino acid residues derived from Glu, Lys, Phe, Tyr, and the D enantiomers thereof;

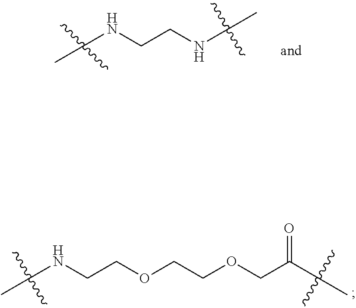 [SMILES: *NCCN* ; *NCCOCCOCC(*)=O]

and at least one group selected from the group consisting of ESL1-R⁵, SSL1-R⁵, ESL2-R⁵ and ESL3-R⁵, 4hBA, 4hPA, 5hPA, and 6hHA, wherein each R⁵ is independently selected from the group consisting of H and C₁₋₄alkyl. and an enzymatically cleavable peptide sequence selected from the group consisting of Met-Val-Lys, Ala-Val, Ala-Pro, Gly-Pro, Ser-Ser, Ser-Gly, Gly-Ala and Ser-Lys.

In some embodiments, one or more of L^{A1}, L^{A2}, L^{A3} and L^{E1} in the conjugate of Formula I-G or I(a)-G are a cleavable linker each independently comprising at least one group selected from the group consisting of ESL1-R⁵, SSL1-R⁵, ESL2-R⁵ ESL3-R⁵, 4hBA, 4hPA, 5hPA, and 6hHA, wherein each R⁵ is independently selected from the group consisting of H and C₁₋₄alkyl, and an enzymatically cleavable peptide sequence selected from the group consisting of Met-Val-Lys, Ala-Val, Ala-Pro, Gly-Pro, Ser-Ser, Ser-Gly, Gly-Ala and Ser-Lys.

In some embodiments, one or more of L^{A1}, L^{A2}, L^{A3} and L^{E1} in the conjugate of Formula I-G are a cleavable linker each independently comprising one or more

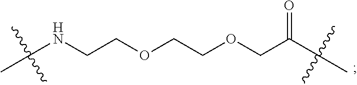 [SMILES: *NCCOCCOCC(*)=O]

and at least one group selected from the group consisting of ESL1-R5, SSL1-R5, ESL2-R5, ESL3-R5, ESL1, ESL2, ESL3, 4hPA-Gly, 5hPA-Gly, 4hBA-Leu, 6hHA-Gly, Gly-5aPOH, I-PADT, and SA-5aPOH, wherein each R⁵ is independently selected from the group consisting of H and C₁₋₄alkyl.

In some embodiments, one or more of L^{A1}, L^{A2}, L^{A3} and L^{E1} in the conjugate of Formula I-G or I(a)-G are an enzymatically cleavable peptide sequence each independently selected from the group consisting of Met-Val-Lys, Ala-Val, Ala-Pro, Gly-Pro, Ser-Ser, Ser-Gly, Gly-Ala and Ser-Lys.

In some embodiments, one or more of L^{A1}, L^{A2}, L^{A3} and L^{E1} in the conjugate of Formula I-G or I(a)-G are a cleavable linker each independently comprising one or more groups selected from the group consisting of one or more amino acid residues derived from Gly, Glu, Leu, Phe, Tyr, or Lys, or the D enantiomers thereof; one or more amino acid residues derived from DAP and DAB; R⁵NC₁₋₂₀alkyleneNR⁶; and R⁵NC₁₋₂₀alkyleneC(O) optionally interrupted by one or more of O; and at least one amino acid residue connected to C(O)C₁₋₁₀alkyleneO or OC₁₋₁₀alkyleneC(O) to form a cleavable moiety selected from the group consisting of C(O)O and OC(O).

In some embodiments, L^{Z1} in the conjugate of Formula I-G or I(a)-G is a non-cleavable linker comprising one or more groups selected from the group consisting of

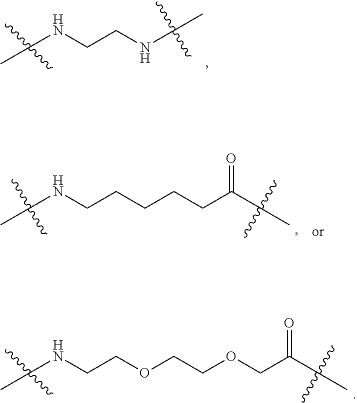 [SMILES: *NCCN* ; *NCCCCCC(*)=O ; *NCCOCCOCC(*)=O]

In some embodiments, A¹ and A in the conjugate of Formula I-G or I(a)-G are both the same. In some embodiments, L^{A1} and L^{A3} in the conjugate of Formula I-G or I(a)-G are the both the same and A¹ and A are both the same.

In some embodiments, L^{A1}, L^{A2} and L^{A3} in the conjugate of Formula I-G or I(a)-G are each independently a direct bond or a non-cleavable linker and L^{E1} in the conjugate of Formula I-G or I(a)-G is a cleavable linker. In some embodiments, L^{A1} and L^{A3} are each independently a direct bond or a non-cleavable linker, L^{A2} is a cleavable linker and L^{E1} is a direct bond or a non-cleavable linker. In some embodiments, L^{A1} and L^{A3} are each independently a cleavable linker and L^{A2} is a direct bond or a non-cleavable linker and L^{E1} is a direct bond or a non-cleavable linker. In some embodiments, L^{A1} and L^{A3} are both the same.

In some embodiments, n is 1, m and o are both 0, R^{Z} is

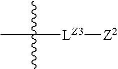

and the conjugate of Formula I-D is a conjugate of Formula I-H

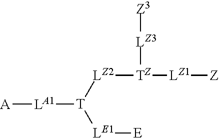

wherein
A, Z, E and T are as defined in Formula I;
Z³ is Z as defined in Formula I and are the same or different;
L^{A1} is L^{A} as defined in Formula I;
L^{Z1}, L^{Z2} and L^{Z3} are each independently L^{Z} as defined in Formula I;
L^{E1} is L^{E} as defined in Formula I.

In some embodiments, at least one of L^{A1} and L^{E1} is a cleavable linker.

In some embodiments, T^{Z} in the conjugate of Formula I-H is a branching group that is at least trivalent comprising at least a first terminal functionality, a second terminal functionality and a third terminal functionality, which are the same or different and bind to L^{Z1} (or alternatively Z), L^{Z2} (or alternatively T) and Z³. In exemplary embodiments, T^{Z} is amino acid residue derived from lysine or an acid residue. In exemplary embodiments, T^{Z} is a trimesic acid residue.

In an exemplary embodiment, E is selected from the group consisting of DOTA and DOTAGA.

In an exemplary embodiment, A in the conjugate of Formula I-H is selected from the group consisting of unsubstituted or substituted C(O)C₁₂₋₁₈alkyleneCOOH.

In some embodiments, one of L^{A1} and L^{E1} in the conjugate of Formula I-H is a non-cleavable linker, and the non-cleavable linker comprises one or more groups selected from the group consisting of one or more amino acids residues derived from Ala, Arg, Asn, Asp, Cys, Gln, Glu, Gly, His, Ile, Leu, Lys, Met, Phe, Pro, Ser, Thr, Trp, Tyr, Val, Pyl, Sec or PCL or the D enantiomers thereof; one or more amino acids residues derived from DAB or DAP; OEG, R¹NC₁₋₂₀alkyleneNR², C(O)C₁₋₂₀alkyleneC(O), R¹NC₁₋₂₀alkyleneC(O), and C(O)C₁₋₂₀alkyleneNR², wherein each R¹ and R² is independently selected from the group consisting of H and C₁₋₂alkyl.

In some embodiments, one of L^{A1} and L^{E1} in the conjugate of Formula I-H is a non-cleavable linker, and the non-cleavable linker comprises one or more groups selected from the group consisting of one or more amino acids residues derived from Ala, Arg, Asn, Asp, Cys, Gln, Glu, Gly, His, Ile, Leu, Lys, Met, Phe, Pro, Ser, Thr, Trp, Tyr, Val, Pyl, Sec, PCL and the D enantiomers thereof; OEG-R¹; Ava-R¹; Aoc-R¹; Aun-R¹ or

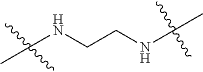

wherein each R¹ is independently selected from the group consisting of H or C₁₋₂alkyl

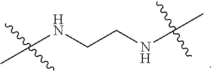 [SMILES: *NCCN*]

In some embodiments, in the conjugate of Formula I-H, one of L^{A1} and L^{E1} is a non-cleavable linker, and the non-cleavable linker comprises one or more groups selected from the group consisting of amino acids residues derived from Glu,

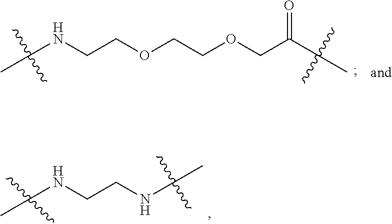 [SMILES: *NCCOCCOCC(*)=O ; *NCCN*]

and the other is a direct bond. In some embodiments, L^{A1} and L^{E1} are each independently a non-cleavable linker, and the non-cleavable linker comprises one or more groups selected from the group consisting of amino acids residues derived from Glu and

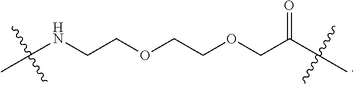 [SMILES: *NCCOCCOCC(*)=O]

In some embodiments, L^{A1} and L^{E1} are the same. In some embodiments, L^{A1} and L^{E1} are comprise two or more of

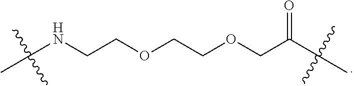 [SMILES: *NCCOCCOCC(*)=O]

In some embodiments, in the conjugate of Formula I-H, one or both of L^{A1} and/or L^{E1} is independently a cleavable linker, and each cleavable linker independently optionally comprises one or more groups selected from the group consisting of one or more amino acid residues derived from Gly, Glu, Leu, Phe, Tyr, or Lys, or the D enantiomers thereof; one or more amino acid residues derived from DAP or DAB; R⁵NC₁₋₂₀alkyleneNR⁶; and R⁵NC₁₋₂₀alkyleneC(O) optionally interrupted by one or more of O; and at least one R⁵NC₁₋₂₀alkyleneC(O) optionally interrupted by one or more of S—S, C(O)O, OC(O), O, C(O)NH and NHC(O), provided R⁵NC₁₋₂₀alkyleneC(O) is interrupted by at least one of S—S, C(O)O and OC(O), or at least one amino acid residue connected to C(O)C₁₋₁₀alkyleneO or OC₁₋₁₀alkyleneC(O) to form a cleavable moiety selected from the group consisting of C(O)O and OC(O), or an enzymatically cleavable peptide sequence selected from the group consisting of Met-Val-Lys, Ala-Val, Ala-Pro, Gly-Pro, Ser-Ser, Ser-Gly, Gly-Ala and Ser-Lys.

In some embodiments, in the conjugate of Formula I-H, one or both of L^{A1} and/or L^{E1} is independently a cleavable linker, and each cleavable linker independently optionally comprises one or more groups selected from the group consisting of amino acid residues derived from Glu, Lys, Phe, Tyr, and the D enantiomers thereof;

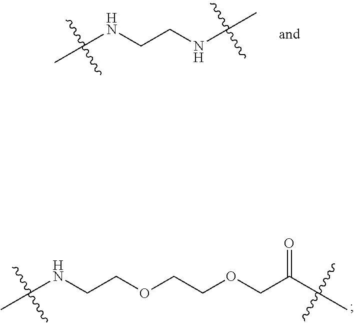 [SMILES: *NCCN* ; *NCCOCCOCC(*)=O]

and at least one group selected from the group consisting of ESL1-R⁵, SSL1-R⁵, ESL2-R⁵ and ESL3-R⁵, 4hBA, 4hPA, 5hPA, and 6hHA, wherein each R⁵ is independently selected from the group consisting of H and C₁₋₄alkyl and an enzymatically cleavable peptide sequence selected from the group consisting of Met-Val-Lys, Ala-Val, Ala-Pro, Gly-Pro, Ser-Ser, Ser-Gly, Gly-Ala and Ser-Lys.

In some embodiments, L^{E1} in the conjugate of Formula I-H is a cleavable linker, and the cleavable linker comprises one or more

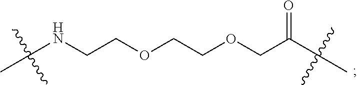 [SMILES: *NCCOCCOCC(*)=O]

and at least one group selected from the group consisting of ESL1-R⁵, SSL1-R⁵, ESL2-R⁵, ESL3-R⁵, 4hBA, 4hPA, 5hPA, and 6hHA, and an enzymatically cleavable peptide sequence, wherein each R⁵ is independently selected from the group consisting of H and C₁₋₄alkyl, and wherein the enzymatically cleavable peptide sequence is selected from the group consisting of Met-Val-Lys, Ala-Val, Ala-Pro, Gly-Pro, Ser-Ser, Ser-Gly, Gly-Ala and Ser-Lys.

In some embodiments, L^{Z1} and L^{Z3} are both the same. In some embodiments, L^{Z1} and/or L^{Z3} each independently further comprise one or more groups selected from the group consisting of

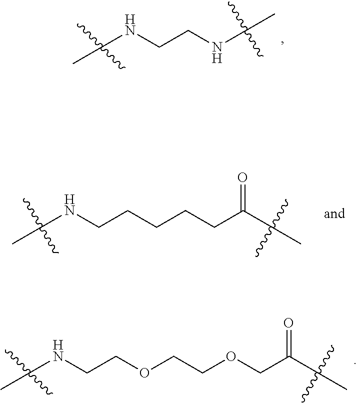 [SMILES: *NCCN* ; *NCCCCCC(*)=O ; *NCCOCCOCC(*)=O]

In some embodiments, L^{Z1} and/or L^{Z3} each independently further comprise one or more

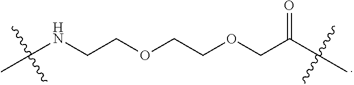 [SMILES: *NCCOCCOCC(*)=O]

in some embodiments, L^{Z1} and L^{Z3 are} both the same.

In some embodiments, L^{Z2} in the conjugate of Formula I-H is a non-cleavable linker comprising one or more groups selected from the group consisting of

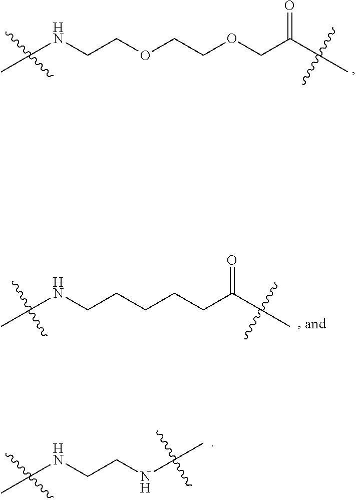 [SMILES: *NCCOCCOCC(*)=O ; *NCCCCCC(*)=O ; *NCCN*]

In some embodiments, L^{A1} is a direct bond or a non-cleavable linker and L^{E1} is a cleavable linker. In some embodiments, L^{A1} is a cleavable linker and L^{E1} is a direct bond or a non-cleavable linker. In some embodiments, L^{Z1} and L^{Z3} are both the same non-cleavable linker.

In some embodiments, Z² and Z in the conjugate of Formula I-H are both the same. In some embodiments, in the conjugate of Formula I-H, L^{Z1} and L^{Z3} are the both the same and Z¹ and Z are both the same.

In some embodiments, o is 1, m and n are both 0, R^{E} is

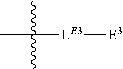

and the conjugate of Formula I-D is a conjugate of Formula I-I

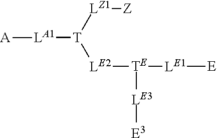

wherein
A, Z, E and T are as defined in Formula I;
E³ is E as defined in Formula I and E³ and E are the same or different;
L^{A1} is L^{A} as defined in Formula I;
L^{E1}, L^{E2} and L^{E3} are each independently L^{E} as defined in Formula I;
L^{A1} is L^{A} as defined in Formula I;
L^{Z1} is L^{Z} as defined in Formula I.

In some embodiments, at least one of L^{E1}, L^{E2} and L^{E3} and L^{A1} is a cleavable linker.

In some embodiments, T^{E} in the conjugate of Formula I-I is a branching group that is at least trivalent comprising at least a first terminal functionality, a second terminal functionality and a third terminal functionality, which are the same or different and bind to L^{E1} (or alternatively E), L^{E2} (or alternatively T) and E³. In exemplary embodiments, T^{E} in the conjugate of Formula I-I is amino acid residue derived from lysine or a trimesic acid residue.

In an exemplary embodiment, E and/or E³ is/are each independently selected from the group consisting of DOTA and DOTAGA.

In an exemplary embodiment, A in the conjugate of Formula I-I is selected from the group consisting of unsubstituted or substituted C(O)C₁₂-18alkyleneCOOH.

In some embodiments, when any one of L^{E1}, L^{E2} and L^{E3} in the conjugate of Formula I-I are a non-cleavable linker, each of the non-cleavable linkers independently comprises one or more groups selected from the group consisting of Ala, Arg, Asn, Asp, Cys, Gln, Glu, Gly, His, Ile, Leu, Lys, Met, Phe, Pro, Ser, Thr, Trp, Tyr, Val, Pyl, Sec or PCL or the D enantiomers thereof; one or more amino acids residues derived from DAB or DAP; R¹NC₁₋₂₀alkyleneNR²; C(O)C₁₋₂₀alkyleneC(O); R¹NC₁₋₂₀alkyleneC(O); and C(O)C₁₋₂₀alkyleneNR², the latter 4 groups being optionally interrupted by one or more of S, O, C(O)NH, NHC(O), C₄₋₆cycloalkyl and C₄₋₆heterocycloalkyl wherein each R¹ and R² is independently selected from the group consisting of H and C₁₋₂alkyl.

In some embodiments, when any one of L^{E1}, L^{E2} and L^{E3} in the conjugate of Formula I-I are a non-cleavable linker, each of the non-cleavable linkers independently comprises one or more groups selected from the group consisting of one or more amino acids residues derived from Ala, Arg, Asn, Asp, Cys, Gln, Glu, Gly, His, Ile, Leu, Lys, Met, Phe, Pro, Ser, Thr, Trp, Tyr, Val, Pyl, Sec, PCL and the D enantiomers thereof; OEG-R¹; Ava-R¹; Aoc-R¹; Aun-R¹ or

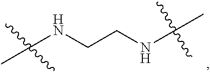

wherein each R¹ is independently selected from the group consisting of H or C₁₋₂alkyl.

In some embodiments, when any one of L^{E1}, L^{E2} and L^{E3} are a non-cleavable linker in the conjugate of Formula I-G, each non-cleavable linker independently comprises one or more groups selected from the group consisting of one or more amino acids residues derived from Ala, Arg, Asn, Asp, Cys, Gln, Glu, Gly, His, Ile, Leu, Lys, Met, Phe, Pro, Ser, Thr, Trp, Tyr, Val, Pyl, Sec, PCL and the D enantiomers thereof; OEG-R¹; Ava-R¹; Aoc-R¹; Aun-R¹ or

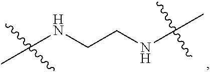

wherein each R¹ is independently selected from the group consisting of H or C₁₋₂alkyl.

In some embodiments, L^{E1} and L^{E3} in the conjugate of Formula I-G are each independently a non-cleavable linker, and each non-cleavable linker independently comprises one or more groups selected from the group consisting of amino acids residues derived from Glu,

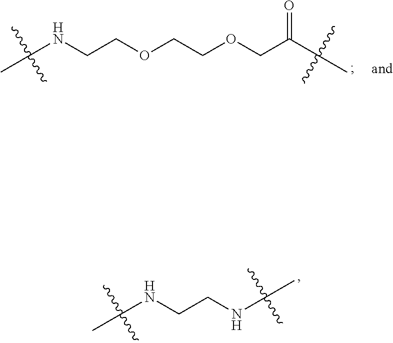 [SMILES: *NCCOCCOCC(*)=O ; *NCCN*]

and H and L^{E2} is a direct bond. In some embodiments, L^{E1} and L^{E3} are each independently a non-cleavable linker, and each non-cleavable linker independently comprises one or more groups selected from the group consisting of amino acids residues derived from Glu and

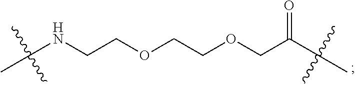 [SMILES: *NCCOCCOCC(*)=O]

and L^{E3} is a direct bond or one or more of

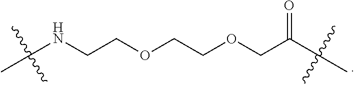 [SMILES: *NCCOCCOCC(*)=O]

In some embodiments, L^{E1} and L^{E3} each independently comprise two or more of

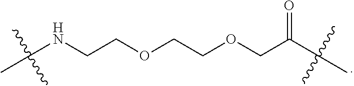 [SMILES: *NCCOCCOCC(*)=O]

In some embodiments, one or more of L^{E1}, L^{E2}, L^{E3} and L^{A1} in the conjugate of Formula I-I is independently a cleavable linker and each cleavable linker independently optionally comprises one or more groups selected from the group consisting of one or more amino acid residues derived from Gly, Glu, Leu, Phe, Tyr, or Lys, or the D enantiomers thereof; one or more amino acid residues derived from DAP and DAB; R⁵NC₁₋₂₀alkyleneNR⁶; and R⁵NC₁₋₂₀alkyleneC(O) optionally interrupted by one or more of O; and at least one R⁵NC₁₋₂₀alkyleneC(O) optionally interrupted by one or more of S—S, C(O)O, OC(O), O, C(O)NH and NHC(O), provided R⁵NC₁₋₂₀alkyleneC(O) is interrupted by at least one of S—S, C(O)O and OC(O), or

at least one amino acid residue connected to C(O)C₁₋₁₀alkyleneO or OC₁₋₁₀alkyleneC(O) to form a cleavable moiety selected from the group consisting of C(O)O and OC(O), or an enzymatically cleavable peptide sequence selected from the group consisting of Met-Val-Lys, Ala-Val, Ala-Pro, Gly-Pro, Ser-Ser, Ser-Gly, Gly-Ala and Ser-Lys.

In some embodiments, one or more of L^{E1}, L^{E2}, L^{E3} and L^{A1} in the conjugate of Formula I-I is independently a cleavable linker and each cleavable linker independently optionally comprises one or more groups selected from the group consisting of amino acid residues derived from Glu, Lys, Phe, Tyr, and the D enantiomers thereof.

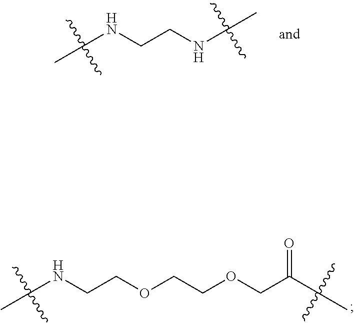 [SMILES: *NCCN* ; *NCCOCCOCC(*)=O]

and N O and at least one group selected from the group consisting of

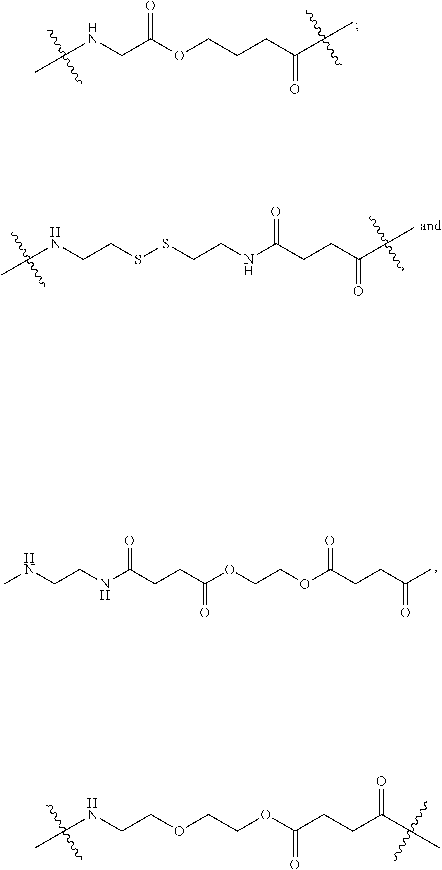 [SMILES: *NCC(=O)OCCCC(*)=O ; *NCCSSCCNC(=O)CCC(*)=O ; CNCCNC(=O)CCC(=O)OCCOC(=O)CCC(C)=O ; *NCCOCCOC(=O)CCC(*)=O]

and an enzymatically cleavable peptide sequence selected from the group consisting of Met-Val-Lys, Ala-Val, Ala-Pro, Gly-Pro, Ser-Ser, Ser-Gly, Gly-Ala and Ser-Lys.

In some embodiments, when L^{A1} in the conjugate of Formula I-I is a cleavable linker, and the cleavable linker optionally comprises one or more

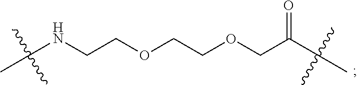 [SMILES: *NCCOCCOCC(*)=O]

and at least one group selected from the group consisting of

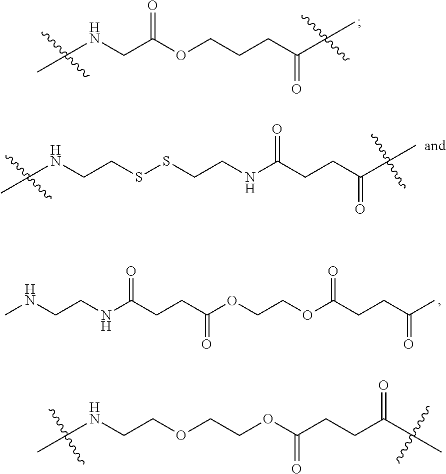 [SMILES: *NCC(=O)OCCCC(*)=O ; *NCCSSCCNC(=O)CCC(*)=O ; CNCCNC(=O)CCC(=O)OCCOC(=O)CCC(C)=O ; *NCCOCCOC(=O)CCC(*)=O]

and an enzymatically cleavable peptide sequence selected from the group consisting of Met-Val-Lys, Ala-Val, Ala-Pro, Gly-Pro, Ser-Ser, Ser-Gly, Gly-Ala and Ser-Lys.

In some embodiments, L^{Z1} in the conjugate of Formula I-I is a direct bond or a non-cleavable linker comprising one or more groups selected from the group consisting of

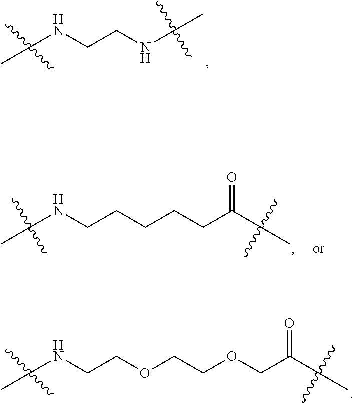 [SMILES: *NCCN* ; *NCCCCCC(*)=O ; *NCCOCCOCC(*)=O]

In some embodiments, L^{E1}, L^{E2} and L^{E3} in the conjugate of Formula I-G are each independently a direct bond or a non-cleavable linker and L^{A1} in the conjugate of Formula I-I is a cleavable linker as defined for L^{A} in Formula I. In some embodiments, L^{E1} and L^{E3}are each independently a direct bond or a non-cleavable linker, L^{E2} is a cleavable linker as defined for L^{A} in Formula I and L^{A1} is a direct bond or a non-cleavable linker. In some embodiments, L^{E1} and L^{E3} are each independently a cleavable linker and L^{E2} is a direct bond or a non-cleavable linker as defined for L^{A} in Formula I and L^{A1} is a direct bond or a non-cleavable liner. In some embodiments, L^{E1} and L^{E3} are both the same.

In some embodiments, E³ and E in the conjugate of Formula I-I are both the same. In some embodiments, L^{E1} and L^{E3} in the conjugate of Formula I-I are the both the same and E³ and E are both the same.

In some embodiments, m is 1, n and o are both 0 and R^{A} is

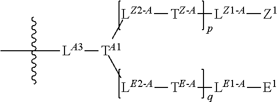

wherein q and p are 0 and the conjugate of Formula I-D is a conjugate of Formula I-J:

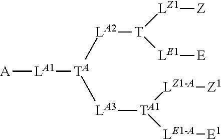

wherein
A, Z, T and E are as defined in Formula I;
E¹ is E as defined in Formula I and E¹ and E are the same or different;
Z¹ is Z as defined in Formula I and Z¹ and Z are the same or different;
L^{A1}, L^{A2} and L^{A3} are each independently L^{A} as defined for in Formula I;
T^{A} and T^{A1} are each independently T as defined for in Formula I and T^{A}, T^{A1} and T are the same or different;
L^{Z1} and L^{Z1-A} are each independently L^{Z} as defined for Formula I;
L^{E1} and L^{E1-A} are each independently L^{E} as defined in Formula I.

In some embodiments, at least one of L^{A1}, L^{A2}, L^{A3}, L^{E1} and L^{E1-A} is a cleavable linker.

In some embodiments, each of T, T^{A}, and T^{A1} is a branching group that is at least trivalent comprising at least a first terminal functionality, a second terminal functionality and a third terminal functionality, which are the same or different and bind to L^{A2} (or alternatively T^{A}), L^{Z1} (or alternatively Z) and L^{E1} (or alternatively E), to L^{A1} (or alternatively A), L^{A2} (or alternatively T) and L^{A3} (or alternatively T^{A1}), and to L^{A3} (or alternatively T^{A}), L^{Z1-A} (or alternatively Z¹) and L^{E1-A} (or alternatively E¹), respectively.

In some embodiments, each of T, T^{A}, and T^{A1} are independently selected from the group consisting of an amino acid residue derived from lysine or glutamine and a trimesic acid residue.

In an exemplary embodiment, A in the conjugate of Formula I-J is selected from the group consisting of unsubstituted or substituted C(O)C₁₂₋₁₈alkyleneCOOH.

In an exemplary embodiment, E¹ and/or E the conjugate of Formula I-J is selected from the group consisting of DOTA and DOTAGA.

In some embodiments, when any of L^{A1}, L^{A2}, L^{A3}, L^{E1} and L^{E1-A} in the conjugate of Formula I-J is a non-cleavable linker, each non-cleavable linkers independently comprises one or more groups selected from the group consisting of Ala, Arg, Asn, Asp, Cys, Gln, Glu, Gly, His, Ile, Leu, Lys, Met, Phe, Pro, Ser, Thr, Trp, Tyr, Val, Pyl, Sec or PCL or the D enantiomers thereof; one or more amino acids residues derived from DAB or DAP; R¹NC₁₋₂₀alkyleneNR²; C(O)C₁₋₂₀alkyleneC(O); R¹NC₁₋₂₀alkyleneC(O); and C(O)C₁₋₂₀alkyleneNR², the latter 4 groups being optionally interrupted by one or more of S, O, C(O)NH, NHC(O), C₄₋₆cycloalkyl and C₄₋₆heterocycloalkyl wherein each R¹ and R² is independently selected from the group consisting of H and C₁₋₂alkyl. In some embodiments, when any of L^{A}, L^{A2}, L^{A3}, L^{E1} and L^{E1-A} in the conjugate of Formula I-J is a non-cleavable linker, each non-cleavable linker independently comprises one or more groups selected from the group consisting of one or more amino acids residues derived from Ala, Arg, Asn, Asp, Cys, Gln, Glu, Gly, His, Ile, Leu, Lys, Met, Phe, Pro, Ser, Thr, Trp, Tyr, Val, Pyl, Sec, PCL and the D enantiomers thereof; OEG-R¹; Ava-R¹; Aoc-R¹; Aun-R¹ or

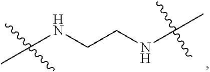

wherein each R¹ is independently selected from the group consisting of H or C₁₋₂alkyl

In some embodiments, when any of L^{A1}, L^{A2}, L^{A3}, L^{E1} and L^{E1-A} in the conjugate of Formula I-J is a non-cleavable linker, each non-cleavable linker independently comprises one or more groups selected from the group consisting of one or more amino acids residues derived from Ala, Arg, Asn, Asp, Cys, Gln, Glu, Gly, His, Ile, Leu, Lys, Met, Phe, Pro, Ser, Thr, Trp, Tyr, Val, Pyl, Sec, PCL and the D enantiomers thereof; OEG-R¹; Ava-R¹; Aoc-R¹; Aun-R¹ or

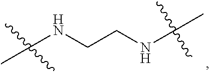

wherein each R¹ is independently selected from the group consisting of H or C₁₋₂alkyl.

In some embodiments, when any of L^{A1}, L^{A2}, L^{A3}, L^{E1} and L^{E1-A} in the conjugate of Formula I-J is a non-cleavable linker, each non-cleavable linker independently comprises one or more groups selected from the group consisting of amino acids residues derived from Glu,

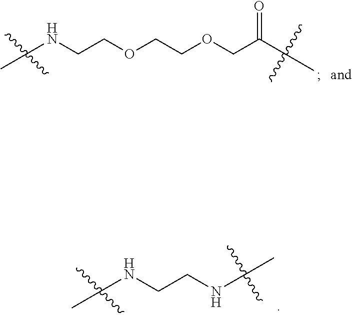 [SMILES: *NCCOCCOCC(*)=O ; *NCCN*]

In some embodiments, when any of L^{A1}, L^{A2}, L^{A3}, L^{E1} and L^{E1-A} in the conjugate of Formula I-J is a non-cleavable linker, each non-cleavable linker independently comprises one or more groups selected from the group consisting of amino acids residues derived from Glu and

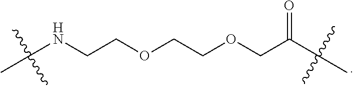 [SMILES: *NCCOCCOCC(*)=O]

In some embodiments, one or more of L^{A1}, L^{A2}, L^{A3}, L^{E1} and L^{E1-A} in the conjugate of Formula I-J are independently a cleavable linker, and each cleavable linker independently optionally comprises one or more groups selected from the group consisting of one or more amino acid residues derived from Gly, Glu, Leu, Phe, Tyr, or Lys, or the D enantiomers thereof; one or more amino acid residues derived from DAP and DAB; R⁵NC₁₋₂₀alkyleneNR⁶; and R⁵NC₁₋₂₀alkyleneC(O) optionally interrupted by one or more of O; and at least one R⁵NC₁₋₂₀alkyleneC(O) optionally interrupted by one or more of S—S, C(O)O, OC(O), O, C(O)NH and NHC(O), provided R⁵NC₁₋₂₀alkyleneC(O) is interrupted by at least one of S—S, C(O)O and OC(O), or

at least one amino acid residue connected to C(O)C₁₋₁₀alkyleneO or OC₁₋₁₀alkyleneC(O) to form a cleavable moiety selected from the group consisting of C(O)O and OC(O), or an enzymatically cleavable peptide sequence selected from the group consisting of Met-Val-Lys, Ala-Val, Ala-Pro, Gly-Pro, Ser-Ser, Ser-Gly, Gly-Ala and Ser-Lys.

In some embodiments, one or more of L^{A1}, L^{A2}, L^{A3}, L^{E1} and L^{E1-A} in the conjugate of Formula I-J are independently a cleavable linker and each cleavable linker independently comprises at least one group selected from the group consisting of ESL1-R5, SSL1-R5, ESL2-R5, ESL3-R5, ESL1, ESL2, ESL3, 4hPA-Gly, 5hPA-Gly, 4hBA-Leu, 6hHA-Gly, Gly-5aPOH, I-PADT, and SA-5aPOH, and an enzymatically cleavable peptide sequence, wherein each R⁵ is independently selected from the group consisting of H and C₁₋₄alkyl, and wherein the enzymatically cleavable peptide sequence is selected from the group consisting of Met-Val-Lys, Ala-Val, Ala-Pro, Gly-Pro, Ser-Ser, Ser-Gly, Gly-Ala and Ser-Lys. In some embodiments, each cleavable linker optionally comprises one or more groups selected from the group consisting of amino acid residues derived from Glu, Lys, Phe, Tyr, and the D enantiomers thereof;

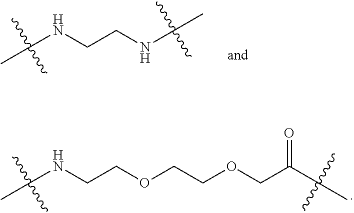 [SMILES: *NCCN* ; *NCCOCCOCC(*)=O]

In some embodiments, one or more of L^{A1}, L^{A2}, L^{A3}, L^{E1} and L^{E1-A} in the conjugate of Formula I-J are independently a cleavable linker, and each cleavable linker independently optionally comprises one or more of

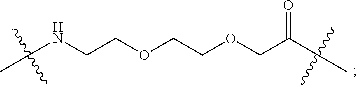 [SMILES: *NCCOCCOCC(*)=O]

and at least one group selected from the group consisting of ESL1-R5, SSL1-R5, ESL2-R5, ESL3-R5, ESL1, ESL2, ESL3, 4hPA-Gly, 5hPA-Gly, 4hBA-Leu, 6hHA-Gly, Gly-5aPOH, I-PADT, and SA-5aPOH, and an enzymatically cleavable peptide sequence, wherein each R⁵ is independently selected from the group consisting of H and C₁₋₄alkyl, and wherein the enzymatically cleavable peptide sequence is selected from the group consisting of Met-Val-Lys, Ala-Val, Ala-Pro, Gly-Pro, Ser-Ser, Ser-Gly, Gly-Ala and Ser-Lys.

In some embodiments, one or more of L^{A1}, L^{A2}, L^{A3}, L^{E1} and/L^{E1-A} in the conjugate of Formula I-J are independently a cleavable linker, and each cleavable linker independently further comprises one or more of

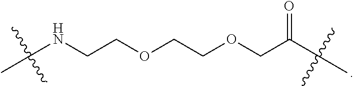 [SMILES: *NCCOCCOCC(*)=O]

In some embodiments, L^{Z1} and L^{Z1}-A in the conjugate of Formula I-J are each independently a non-cleavable linker comprising one or more groups selected from the group consisting of

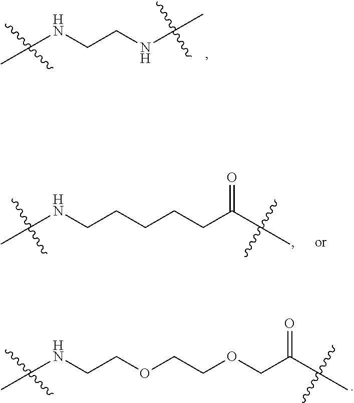 [SMILES: *NCCN* ; *NCCCCCC(*)=O ; *NCCOCCOCC(*)=O]

In some embodiments, L^{E1}, L^{E1-A}, L^{A1}, L^{Z1} and L^{Z1-A} are each independently a direct bond or a non-cleavable linker, and L^{A2} and L^{A3} are each independently cleavable linkers.

In some embodiments, L^{E1}, L^{E1-A} L^{A1}, L^{Z1} and L^{Z1}-A are each independently a direct bond or a non-cleavable linker, and one of L^{A2} and L^{A3} is a non-cleavable linked and the other is a cleavable linker. In some embodiments, L^{A1}, is a cleavable linker and L^{E1}, L^{E1-A}, L^{Z1}, L^{Z1-A}, L^{A2} and L^{A3} are each independently a direct bond or a non-cleavable linker. In some embodiments, L^{A2} and L^{A3} are both the same. In some embodiments, L^{Z1} and L^{Z1}-A are both the same. In some embodiments, L^{E1} and L^{E1-A} are both the same.

In some embodiments, o is 1, n and m are both 0 and R^{E} is

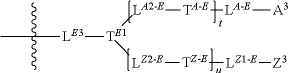

wherein t and u are 0 and the conjugate of Formula I-D is a conjugate of Formula I-K:

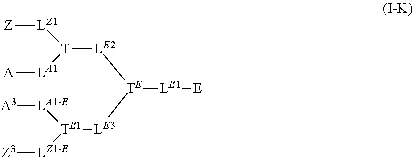

wherein
A, Z, T and E are as defined in Formula I
A³ is A as defined in Formula I and A³ and A are the same or different;
Z³ is Z as defined in Formula I and Z³ and Z are the same or different;
L^{A1} and L^{A1-E} are each independently L^{A} as defined for in Formula I;
T^{E} and T^{E1} are each independently T as defined for in Formula I and T, T^{E} and T^{E1} are the same or different;
L^{Z1} and L^{Z1-E} are each independently L^{Z} as defined for Formula I;
L^{E1}, L^{E2}, L^{E3} are each independently L^{E} as defined in Formula I.

In some embodiments, at least one of L^{E1}, L^{E2}, L^{E3}, L^{A1} and L^{A1-E} is a cleavable linker.

In some embodiments, each of T, T^{E} and T^{E1} is a branching group that is at least trivalent comprising at least a first terminal functionality, a second terminal functionality and a third terminal functionality, which are the same or different and bind to L^{A1} (or alternatively A), L^{Z1} (or alternatively Z) and L^{E2} (or alternatively T^{E}), L^{E1} (or alternatively E), L^{E2} (or alternatively T) and L^{A3} (or alternatively T^{E}l), and to L^{E3} (or alternatively T^{E}) L^{A1-E} (or alternatively A³) and L^{Z1-2} (or alternatively Z³), respectively.

In some embodiments, each of T, T^{E} and T^{E1} are independently selected from the group consisting of an amino acid residue derived from lysine or glutamine and a trimesic acid residue.

In an exemplary embodiment, A and/or A³ in the conjugate of Formula I-K is selected from the group consisting of unsubstituted or substituted C(O)C₁₂₋₁₈alkyleneCOOH.

In an exemplary embodiment, E the conjugate of Formula I-K is selected from the group consisting of DOTA and DOTAGA.

In some embodiments, when any one or more of L^{E1}, L^{E2}, L^{E3}, L^{A1} and L^{A1-E} in the conjugate of Formula I-K is a non-cleavable linker, each non-cleavable linker independently comprises one or more groups selected from the group consisting of one or more amino acids residues derived from Ala, Arg, Asn, Asp, Cys, Gln, Glu, Gly, His, Ile, Leu, Lys, Met, Phe, Pro, Ser, Thr, Trp, Tyr, Val, Pyl, Sec, PCL and the D enantiomers thereof; OEG-R¹; Ava-R¹; Aoc-R¹; Aun-R¹ or

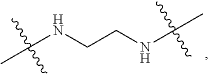

wherein each R¹ is independently selected from the group consisting of H or C₁₋₂alkyl

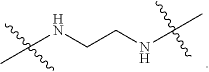 [SMILES: *NCCN*]

In some embodiments, when any of L^{E1}, L^{E2}, L^{E3}, L^{A1} and L^{A1-E} in the conjugate of Formula I-K is a non-cleavable linker, each non cleavable linkers independently comprises one or more groups selected from the group consisting of Ala, Arg, Asn, Asp, Cys, Gln, Glu, Gly, His, Ile, Leu, Lys, Met, Phe, Pro, Ser, Thr, Trp, Tyr, Val, Pyl, Sec or PCL or the D enantiomers thereof; one or more amino acids residues derived from DAB or DAP; R¹NC₁₋₂₀alkyleneNR²; C(O)C₁₋₂₀alkyleneC(O); R¹NC₁₋₂₀alkyleneC(O); and C(O)C₁₋₂₀alkyleneNR², the latter 4 groups being optionally interrupted by one or more of S, O, C(O)NH, NHC(O), C₄₋₆cycloalkyl and C₄₋₆heterocycloalkyl wherein each R¹ and R² is independently selected from the group consisting of H and C₁₋₂alkyl. In some embodiments, when any of L^{E1}, L^{E2}, L^{E3}, L^{A1} and L^{A1-E} in the conjugate of Formula I-K is a non-cleavable linker, each non-cleavable linker independently one or more groups selected from the group consisting of one or more amino acids residues derived from Ala, Arg, Asn, Asp, Cys, Gln, Glu, Gly, His, Ile, Leu, Lys, Met, Phe, Pro, Ser, Thr, Trp, Tyr, Val, Pyl, Sec, PCL and the D enantiomers thereof; OEG-R¹; Ava-R¹; Aoc-R¹; Aun-R¹ or

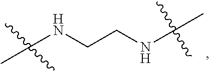

wherein each R¹ is independently selected from the group consisting of H or C₁₋₂alkyl

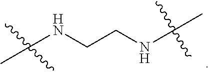 [SMILES: *NCCN*]

In some embodiments, when any one of L^{E1}, L^{E2}, L^{E3}, L^{A} and L^{A1-E} in the conjugate of Formula I-K is a non-cleavable linker, each non-cleavable linker independently one or more groups selected from the group consisting of amino acids residues derived from Glu,

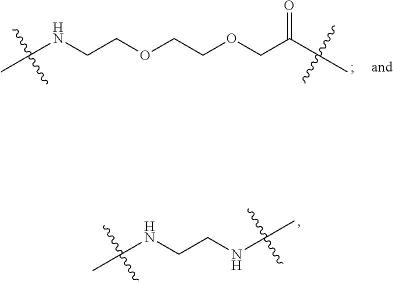 [SMILES: *NCCOCCOCC(*)=O ; *NCCN*]

In some embodiments, when any of L^{E1}, L^{E2}, L^{E3}, L^{A1} and L^{A1-E} in the conjugate of Formula I-K is a non-cleavable linker, each non-cleavable linker independently comprises one or more groups selected from the group consisting of amino acids residues derived from Glu and

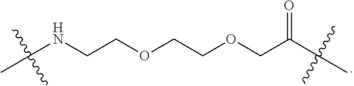 [SMILES: *NCCOCCOCC(*)=O]

In some embodiments, one or more of L^{E1}, L^{E2}, L^{E3}, L^{A1} and L^{A1-E} in the conjugate of Formula I-K are independently a cleavable linker and each cleavable linker independently comprises one or more groups selected from the group consisting of one or more amino acid residues derived from Gly, Glu, Leu, Phe, Tyr, or Lys, or the D enantiomers thereof; one or more amino acid residues derived from DAP and DAB; R⁵NC₁₋₂₀alkyleneNR⁶; and R⁵NC₁₋₂₀alkyleneC(O) optionally interrupted by one or more of O; and at least one R⁵NC₁₋₂₀alkyleneC(O) optionally interrupted by one or more of S—S, C(O)O, OC(O), O, C(O)NH and NHC(O), provided R⁵NC₁₋₂₀alkyleneC(O) is interrupted by at least one of S—S, C(O)O and OC(O), or

at least one amino acid residue connected to C(O)C₁₋₁₀alkyleneO or OC₁₋₁₀alkyleneC(O) to form a cleavable moiety selected from the group consisting of C(O)O and OC(O), or an enzymatically cleavable peptide sequence selected from the group consisting of Met-Val-Lys, Ala-Val, Ala-Pro, Gly-Pro, Ser-Ser, Ser-Gly, Gly-Ala and Ser-Lys.

In some embodiments, one or more of L^{E1}, L^{E2}, L^{E3}, L^{A1} and L^{A1-E} in the conjugate of Formula I-K are independently a cleavable linker and each cleavable linker independently comprises at least one group selected from the group consisting of ESL1-R5, SSL1-R5, ESL2-R5, ESL3-R5, ESL1, ESL2, ESL3, 4hPA-Gly, 5hPA-Gly, 4hBA-Leu, 6hHA-Gly, Gly-5aPOH, I-PADT, and SA-5aPOH, and an enzymatically cleavable peptide sequence, wherein each R⁵ is independently selected from the group consisting of H and C₁₋₄alkyl, and wherein the enzymatically cleavable peptide sequence is selected from the group consisting of Met-Val-Lys, Ala-Val, Ala-Pro, Gly-Pro, Ser-Ser, Ser-Gly, Gly-Ala and Ser-Lys. In some embodiments, each cleavable linker optionally comprises one or more groups selected from the group consisting of amino acid residues derived from Glu, Lys, Phe, Tyr, and the D enantiomers thereof;

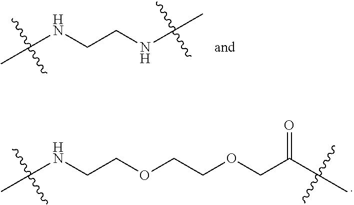 [SMILES: *NCCN* ; *NCCOCCOCC(*)=O]

In some embodiments, one or more of L^{E1}, L^{E2}, L^{E3}, L^{A1} and L^{A1-E} in the conjugate of Formula I-K are independently a cleavable linker, and each cleavable linker independently optionally comprises one or more of

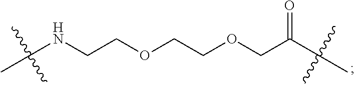 [SMILES: *NCCOCCOCC(*)=O]

and at least one group selected from the group consisting of ESL1-R⁵, SSL1-R⁵, ESL2-R⁵, ESL3-R⁵, 4hBA, 4hPA, 5hPA, and 6hHA, and an enzymatically cleavable peptide sequence, wherein each R⁵ is independently selected from the group consisting of H and C₁₋₄alkyl, and wherein the enzymatically cleavable peptide sequence is selected from the group consisting of Met-Val-Lys, Ala-Val, Ala-Pro, Gly-Pro, Ser-Ser, Ser-Gly, Gly-Ala and Ser-Lys.

In some embodiments, one or more of L^{E1}, L^{E2}, L^{E3}, L^{A1} and L^{A1-E} in the conjugate of Formula I-K are a cleavable linker, and each cleavable linker optionally further comprises one or more of

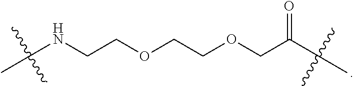 [SMILES: *NCCOCCOCC(*)=O]

In some embodiments, L^{Z1} and L^{Z1-E} in the conjugate of Formula I-K are each independently a non-cleavable linker comprising one or more groups selected from the

group consisting of

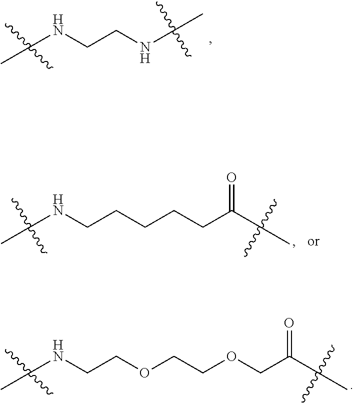 [SMILES: *NCCN* ; *NCCCCCC(*)=O ; *NCCOCCOCC(*)=O]

In some embodiments, L^{A1}, L^{A1-E}, L^{E1}, L^{Z1} and L^{Z1-E} are each independently a direct bond or a non-cleavable linker, and L^{E2} and L^{E3} are each independently cleavable linkers.

In some embodiments, L^{A1}, L^{A1-E} L^{E1}, L^{Z1} and L^{Z1-E} are each independently a direct bond or a non-cleavable linker, and one of L^{E2} and L^{E3} is a non-cleavable linked and the other is a cleavable linker. In some embodiments, L^{E1} is a cleavable linker and L^{A1}, L^{A1-E}, L^{Z1}, L^{Z1-E}, L^{E2} and L^{E3} are each independently a direct bond or a non-cleavable linker. In some embodiments, L^{E2} and L^{E3} are both the same. In some embodiments, L^{Z1} and L^{Z1-E} are both the same. In some embodiments, L^{A1} and L^{A1-E} are both the same.

In some embodiments, n is 1, m and o are both 0 and R^{Z} is

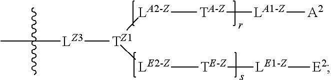

wherein r and s are 0 and the conjugate of Formula I-D is a conjugate of Formula I-L:

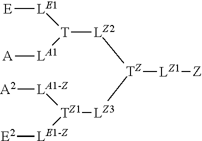

wherein
A, Z, T and E are as defined in Formula I
A² is A as defined in Formula I and A² and A are the same or different;
E² is E as defined in Formula I and E² and E are the same or different;
L^{A1} and L^{A1-Z} are each independently L^{A} as defined for in Formula I;
T^{Z} and T^{Z1} are each independently T as defined for in Formula I and T, T^{Z} and T^{Z1} are the same or different;
L^{A1} and L^{A1-Z} are each independently L^{E} as defined for Formula I;
L^{E1} and L^{E1-Z} are each independently L^{E} as defined for Formula I;
L^{Z1}, L^{Z2} and L^{Z3} are each independently L^{Z} as defined in Formula 1.

In some embodiments, at least one of L^{A1}, L^{A-Z}, L^{E1} and L^{E1-Z} is a cleavable linker.

In some embodiments, each of T, T^{Z} and T^{Z1} is a branching group that is at least trivalent comprising at least a first terminal functionality, a second terminal functionality and a third terminal functionality, which are the same or different and bind to L^{A1} (or alternatively A), L^{E1} (or alternatively E) and L^{Z2} (or alternatively T^{Z}), L^{Z1} (or alternatively Z), L^{Z2} (or alternatively T) and L^{Z3} (or alternatively T^{Z1}), and to L^{Z3} (or alternatively T^{Z}), L^{A1-Z} (or alternatively A²) and L^{E1-Z} (or alternatively E²), respectively.

In some embodiments, each of T, T^{Z} and T^{Z1} are independently selected from the group consisting of an amino acid residue derived from lysine or glutamine and a trimesic acid residue.

In an exemplary embodiment, A and/or A² in the conjugate of Formula I-L are independently selected from the group consisting of unsubstituted or substituted C(O)C₁₂₋₁₈alkyleneCOOH.

In an exemplary embodiment, E and/or E² the conjugate of Formula I-L is selected from the group consisting of DOTA and DOTAGA.

In some embodiments, when any of L^{A1}, L^{A1-Z}, L^{E1} and L^{E1-Z} in the conjugate of Formula I-L is a non-cleavable linker, each non-cleavable linker independently comprises one or more groups selected from the group consisting of Ala, Arg, Asn, Asp, Cys, Gln, Glu, Gly, His, Ile, Leu, Lys, Met, Phe, Pro, Ser, Thr, Trp, Tyr, Val, Pyl, Sec or PCL or the D enantiomers thereof; one or more amino acids residues derived from DAB or DAP; R¹NC₁₋₂₀alkyleneNR²; C(O)C₁₋₂₀alkyleneC(O); R¹NC₁₋₂₀alkyleneC(O); and C(O)C₁₋₂₀alkyleneNR², the latter 4 groups being optionally interrupted by one or more of S, O, C(O)NH, NHC(O), C₄₋₆cycloalkyl and C₄₋₆heterocycloalkyl wherein each R¹ and R² is independently selected from the group consisting of H and C₁₋₂alkyl. In some embodiments, when any of L^{A1}, L^{A1-Z}, L^{E1} and L^{E1-Z} in the conjugate of Formula I-L is a non-cleavable linker, each non-cleavable linker independently comprises one or more groups selected from the group consisting of one or more amino acids residues derived from Ala, Arg, Asn, Asp, Cys, Gln, Glu, Gly, His, Ile, Leu, Lys, Met, Phe, Pro, Ser, Thr, Trp, Tyr, Val, Pyl, Sec, PCL and the D enantiomers thereof; OEG-R¹; Ava-R¹; Aoc-R¹; Aun-R¹ or

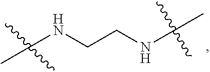

wherein each R¹ is independently selected from the group consisting of H or C₁₋₂alkyl

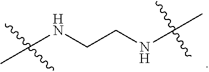 [SMILES: *NCCN*]

In some embodiments, when any of L^{A1}, L^{A-Z}, L^{E1} and L^{E1-Z} in the conjugate of Formula I-L is a non-cleavable linker, each non-cleavable linker independently comprises one or more groups selected from the group consisting of one or more amino acids residues derived from Ala, Arg, Asn, Asp, Cys, Gln, Glu, Gly, His, Ile, Leu, Lys, Met, Phe, Pro, Ser, Thr, Trp, Tyr, Val, Pyl, Sec, PCL and the D enantiomers thereof; OEG-R¹; Ava-R¹; Aoc-R¹; Aun-R¹ or

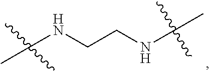

wherein each R¹ is independently selected from the group consisting of H or C₁₋₂alkyl.

In some embodiments, when any of L^{A1}, L^{A1-Z}, L^{E1} and L^{E1-Z} in the conjugate of Formula I-L is a non-cleavable linker, each non-cleavable linker independently comprises one or more groups selected from the group consisting of amino acids residues derived from Glu,

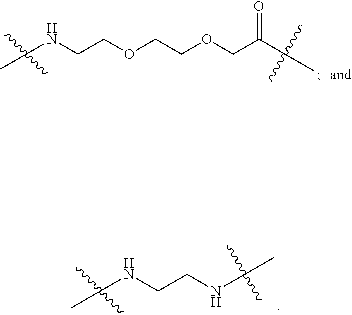 [SMILES: *NCCOCCOCC(*)=O ; *NCCN*]

In some embodiments, when any of L^{A1}, L^{A1-Z}, L^{E1} and L^{E1-Z} in the conjugate of Formula I-L is a non-cleavable linker each non-cleavable linker independently comprises one or more groups selected from the group consisting of amino acids residues derived from Glu and

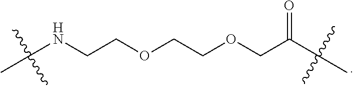 [SMILES: *NCCOCCOCC(*)=O]

In some embodiments, one or more of L^{A1}, L^{A1-Z}, L^{E1} and L^{E1-Z} in the conjugate of Formula I-L is independently a cleavable linker and each cleavable linker independently comprises one or more groups selected from the group consisting of one or more amino acid residues derived from Gly, Glu, Leu, Phe, Tyr, or Lys, or the D enantiomers thereof; one or more amino acid residues derived from DAP and DAB; R⁵NC₁₋₂₀alkyleneNR⁶; and R⁵NC₁₋₂₀alkyleneC(O) optionally interrupted by one or more of O; and at least one R⁵NC₁₋₂₀alkyleneC(O) optionally interrupted by one or more of S—S, C(O)O, OC(O), O, C(O)NH and NHC(O), provided R⁵NC₁₋₂₀alkyleneC(O) is interrupted by at least one of S—S, C(O)O and OC(O), or

at least one amino acid residue connected to C(O)C₁₋₁₀alkyleneO or OC₁₋₁₀alkyleneC(O) to form a cleavable moiety selected from the group consisting of C(O)O and OC(O), or an enzymatically cleavable peptide sequence selected from the group consisting of Met-Val-Lys, Ala-Val, Ala-Pro, Gly-Pro, Ser-Ser, Ser-Gly, Gly-Ala and Ser-Lys.

In some embodiments, one or more of L^{A1}, L^{A1-Z}, L^{E1} and L^{E1-Z} in the conjugate of Formula I-L is independently a cleavable linker and each cleavable linker independently comprises at least one group selected from the group consisting of ESL1-R5, SSL1-R5, ESL2-R5, ESL3-R5, ESL1, ESL2, ESL3, 4hPA-Gly, 5hPA-Gly, 4hBA-Leu, 6hHA-Gly, Gly-5aPOH, I-PADT, and SA-5aPOH, and an enzymatically cleavable peptide sequence, wherein each R⁵ is independently selected from the group consisting of H and C₁₋₄alkyl, and wherein the enzymatically cleavable peptide sequence is selected from the group consisting of Met-Val-Lys, Ala-Val, Ala-Pro, Gly-Pro, Ser-Ser, Ser-Gly, Gly-Ala and Ser-Lys. In some embodiments, each cleavable linker optionally comprises one or more groups selected from the group consisting of amino acid residues derived from Glu, Lys, Phe, Tyr, and the D enantiomers thereof;

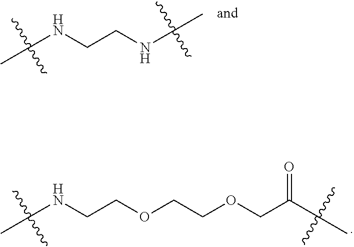 [SMILES: *NCCN* ; *NCCOCCOCC(*)=O]

In some embodiments, one or more of L^{A1}, L^{A1-Z}, L^{E1} and L^{E1-Z} in the conjugate of Formula I-L is independently a cleavable linker, and each cleavable linker independently optionally comprises one or more of

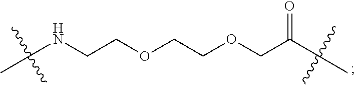 [SMILES: *NCCOCCOCC(*)=O]

and at least one group selected from the group consisting of ESL1-R5, SSL1-R5, ESL2-R5, ESL3-R5, ESL1, ESL2, ESL3, 4hPA-Gly, 5hPA-Gly, 4hBA-Leu, 6hHA-Gly, Gly-5aPOH, I-PADT, and SA-5aPOH, and an enzymatically cleavable peptide sequence, wherein each R⁵ is independently selected from the group consisting of H and C₁₋₄alkyl, and wherein the enzymatically cleavable peptide sequence is selected from the group consisting of Met-Val-Lys, Ala-Val, Ala-Pro, Gly-Pro, Ser-Ser, Ser-Gly, Gly-Ala and Ser-Lys.

In some embodiments, at least one of L^{A1}, L^{A1-Z} L^{E1} and L^{E1-Z} in the conjugate of Formula I-L is a cleavable linker, and each cleavable linker independently further comprises one or more of

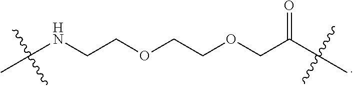 [SMILES: *NCCOCCOCC(*)=O]

In some embodiments, L^{Z1}, L^{Z2} and L^{Z3} in the conjugate of Formula I-L are each independently a non-cleavable linker comprising one or more groups selected from the group consisting of

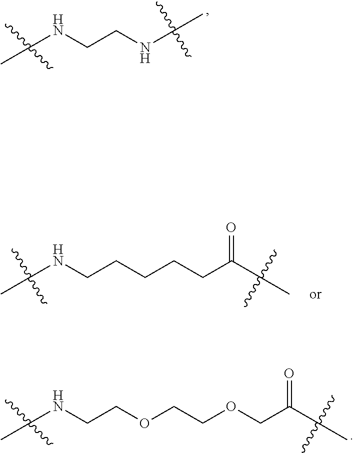 [SMILES: *NCCN* ; *NCCCCCC(*)=O ; *NCCOCCOCC(*)=O]

In some embodiments, L^{Z2} and L^{Z3} in the conjugate of Formula I-L are each independently a non-cleavable linker comprising one or more groups selected from the group consisting of

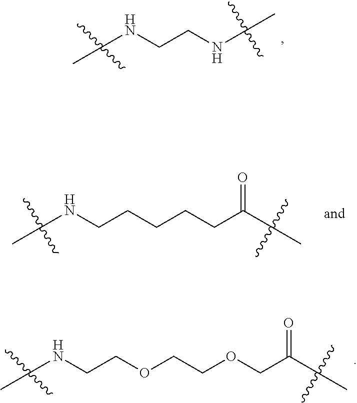 [SMILES: *NCCN* ; *NCCCCCC(*)=O ; *NCCOCCOCC(*)=O]

In some embodiments, L^{Z1} in the conjugate of Formula I-L are each independently a non-cleavable linker comprising one or more groups selected from the group consisting of

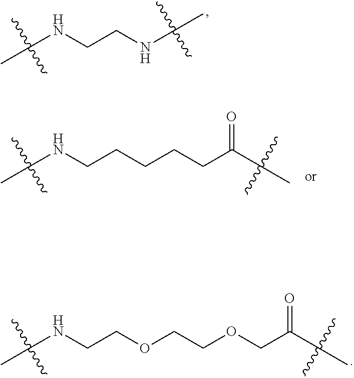 [SMILES: *NCCN* ; *NCCCCCC(*)=O ; *NCCOCCOCC(*)=O]

In some embodiments, L^{Z1}, L^{Z2}, L^{Z3}, L^{E1} and L^{E1-Z} are each independently a direct bond or a non-cleavable linker, and L^{A1} and L^{A1-Z}, are each independently cleavable linkers. In some embodiments, L^{Z1}, L^{Z2}, L^{Z3}, L^{E1} and L^{E1-Z} are each independently a direct bond or a non-cleavable linker, and one of L^{A1} and L^{A1-Z} is a non-cleavable linked and the other is a cleavable linker. In some embodiments, L^{Z1}, L^{Z2}, L^{Z3}, L^{A1} and L^{A1-Z} are each independently a direct bond or a non-cleavable linker, and L^{E1} and L^{E1-Z} are each independently cleavable linkers. In some embodiments, L^{Z1}, L^{Z2}, L^{Z3}, L^{A1} and L^{A1-Z} are each independently a direct bond or a non-cleavable linker, and one of L^{E1} and L^{E1-Z} is a non-cleavable linked and the other is a cleavable linker. In In some embodiments, L^{Z2} and L^{Z3} are both the same. In some embodiments, L^{E1} and L^{E1-Z} are both the same. In some embodiments, L^{A1} and L^{A1-Z} are both the same.

In some embodiments, the conjugate of Formula I is selected from the group consisting of any one of the conjugates listed in Table 3.

TABLE 3
Exemplary conjugates of the present application

| No. | Seq/Structure |
|---|---|
| C001 | HO—C18-gGlu-OEG-OEG-ESL1-Lys(DOTAGA)-Lys(PEG3-Suc-Z)-Tyr-NH2 |
| | 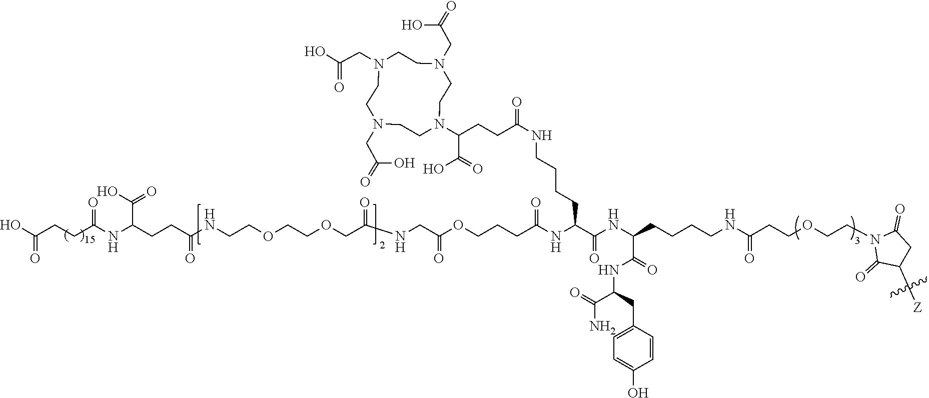 |
| C002 | HO—C18-gGlu-OEG-OEG-Lys(ESL1-DOTAGA)-Lys(PEG3-Suc-Z)-Tyr-NH2 |
| | 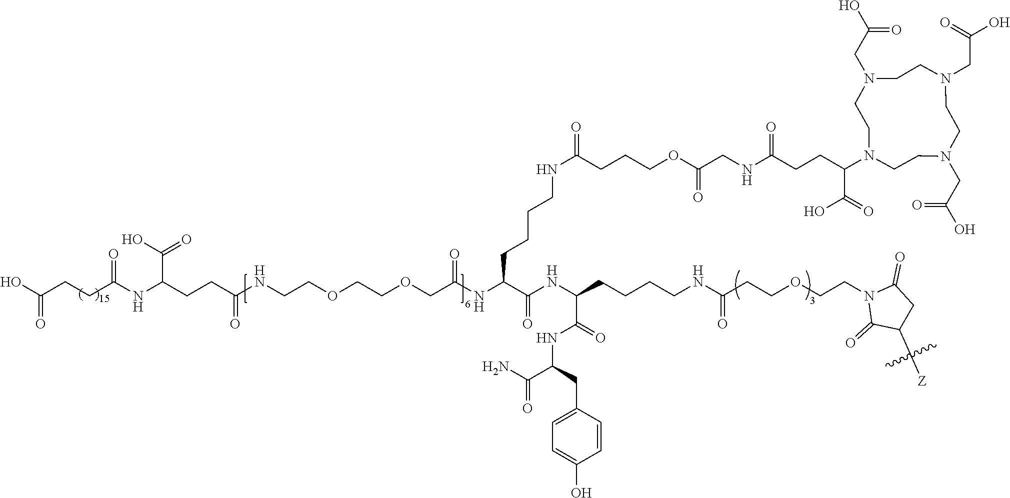 |
| C003 | HO—C20-gGlu-OEG-OEG-OEG-OEG-OEG-OEG-ESL1-Lys(DOTAGA)-Lys(PEG3-Suc-Z)-Tyr-NH2 |
| | 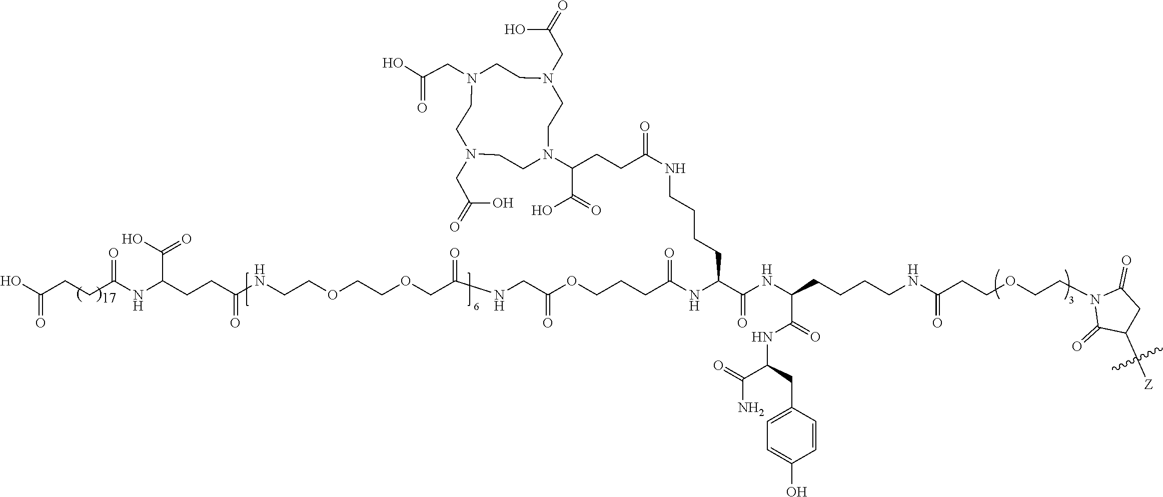 |
| C004 | HO—C20-gGlu-OEG-OEG-OEG-OEG-OEG-OEG-Lys(ESL1-DOTAGA)-Lys(PEG3-Suc-Z)-Tyr-NH2 |
| | 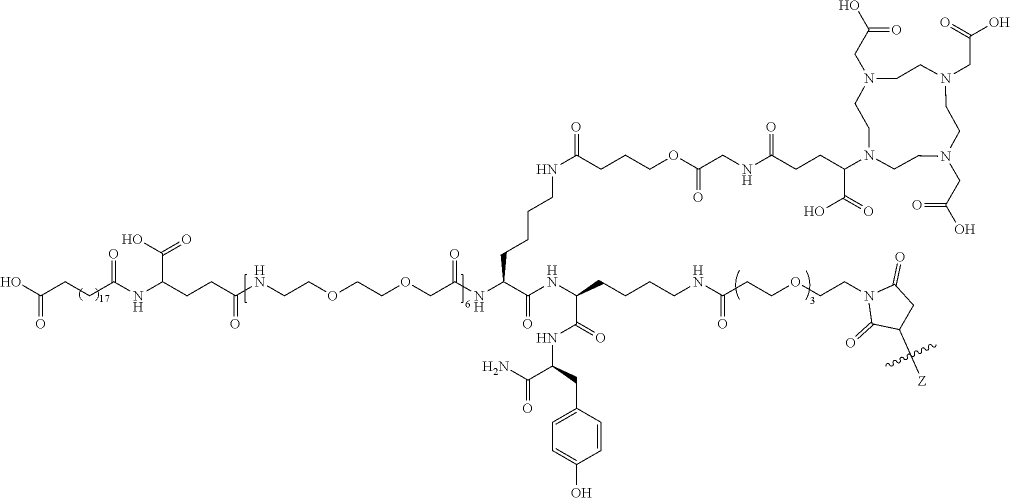 |
| C005 | HO—C18-gGlu-OEG-OEG-OEG-Lys(ESL1-DOTAGA)-OEG-OEG-OEG-Lys[CO—CH2—C6H4-(p-PTU-Z)]—NH2 |
| | 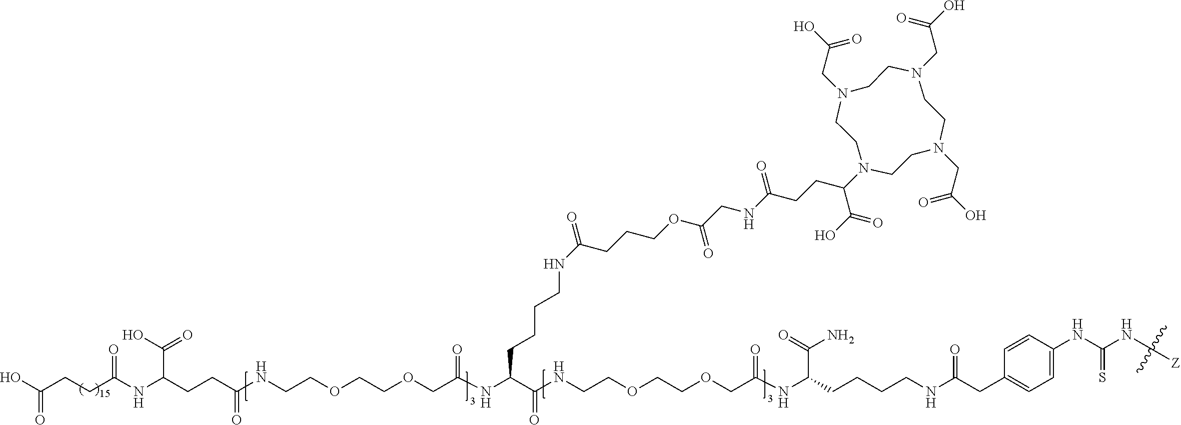 |
| C006 | IPhBu-gGlu-PEG8-ESL1-Lys(DOTAGA)-Lys(PEG3-Suc-Z)-Tyr-NH2 |
| | 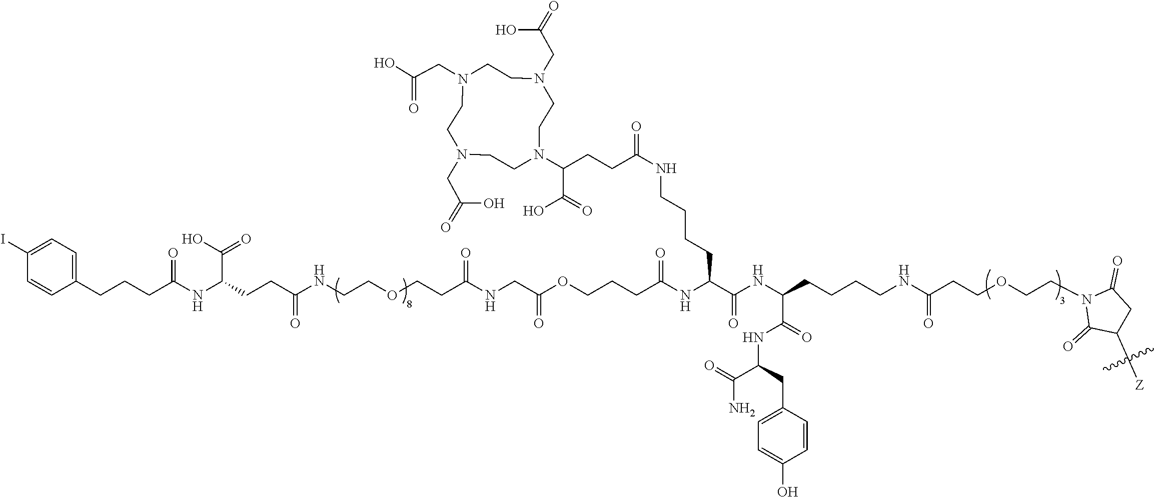 |
| C007 | IPhBu-gGlu-PEG8-Lys(ESL1-DOTAGA)-Lys(PEG3-Suc-Z)-Tyr-NH2 |
| | 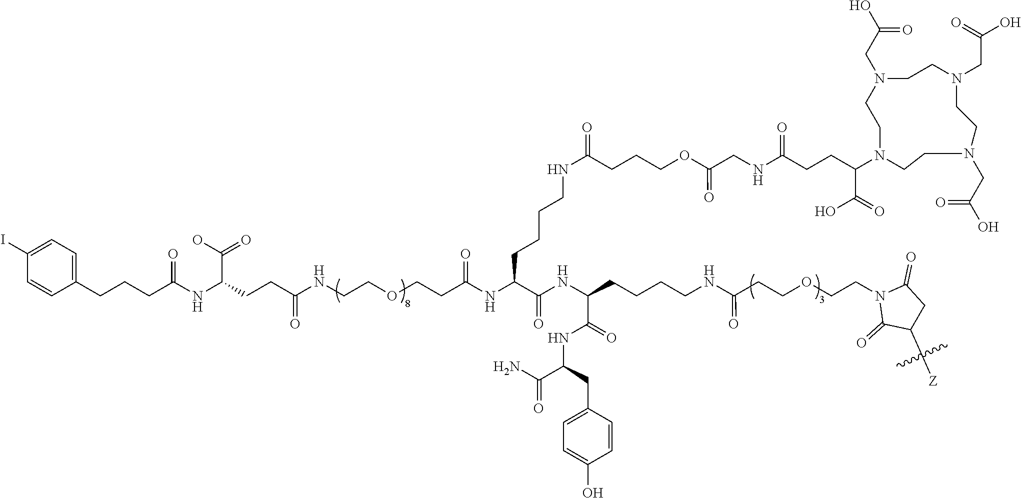 |
| C008 | C18-gGlu-OEG-OEG-OEG-Lys(ESL1-DOTAGA)-OEG-OEG-OEG-Lys[CO—CH2—C6H4-(p-PTU-Z)]—NH2 |
| | 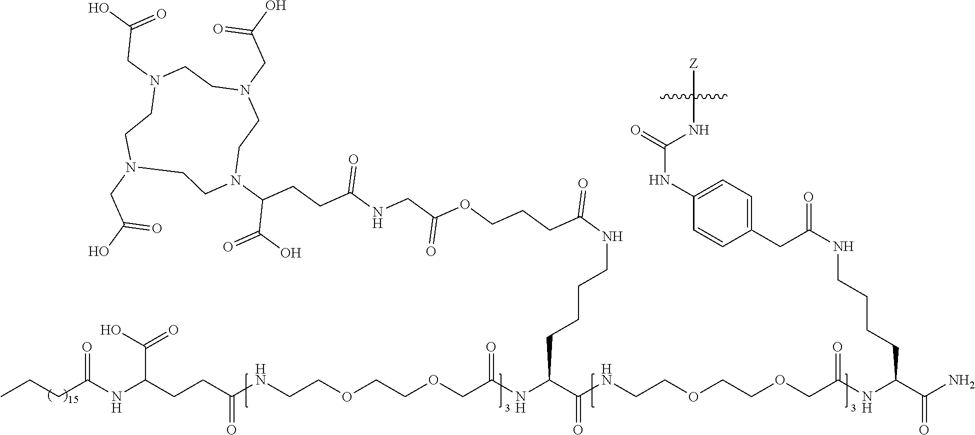 |
| C009 | HO—C18-gGlu-Aun-Aun-Lys(ESL1-DOTAGA)-Lys(PEG3-Suc-Z)-Tyr-NH2 |
| | 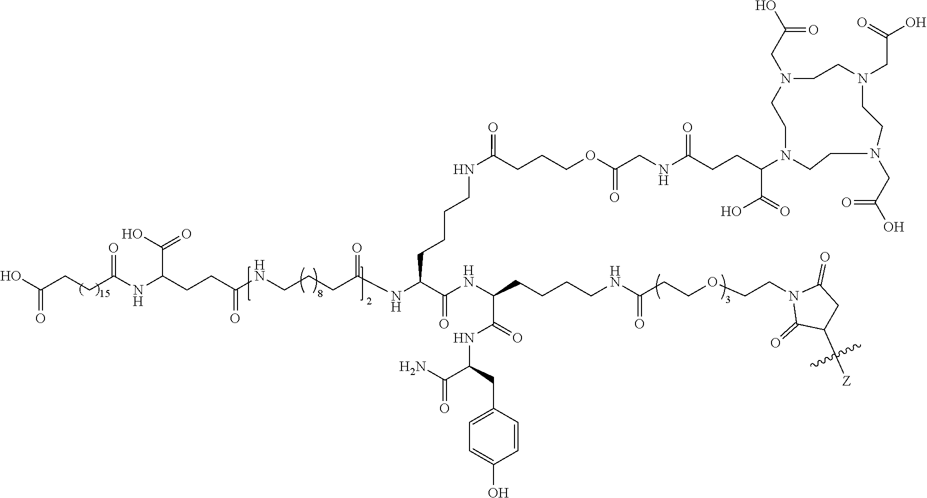 |
| C010 | HO—C18-gGlu-gGlu-gGlu-gGlu-Lys(ESL1-DOTAGA)-Lys(PEG3-Suc-Z)-Tyr-NH2 |
| | 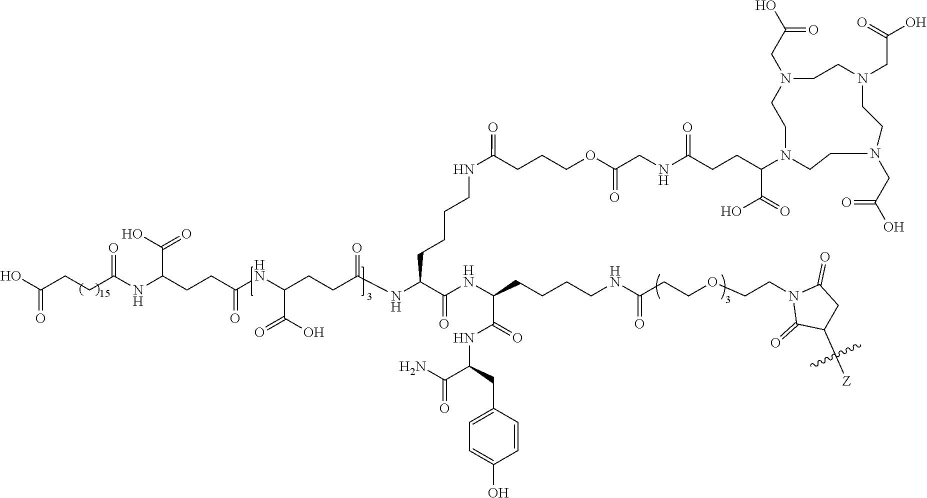 |
| C011 | HO—C18-gGlu-OEG-OEG-Lys(C18)-ESL1-Lys(DOTAGA)-Lys(PEG3-Suc-Z)-Tyr-N |
| | 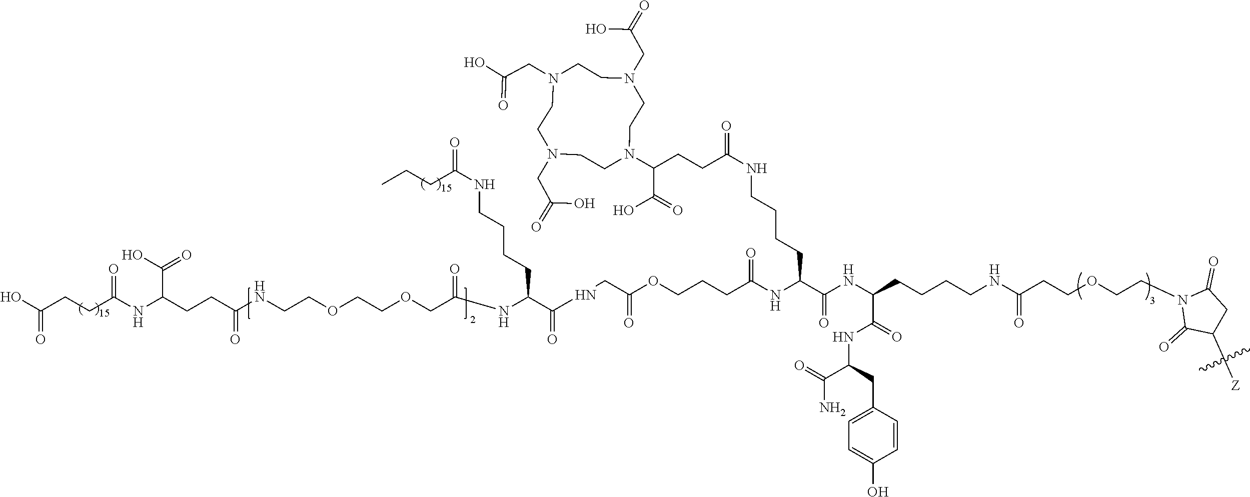 |
| C012 | HO—C18-gGlu-OEG-OEG-Lys(C14)-ESL1-Lys(DOTAGA)-Lys(PEG3-Suc-Z)-Tyr-NH2 |
| | 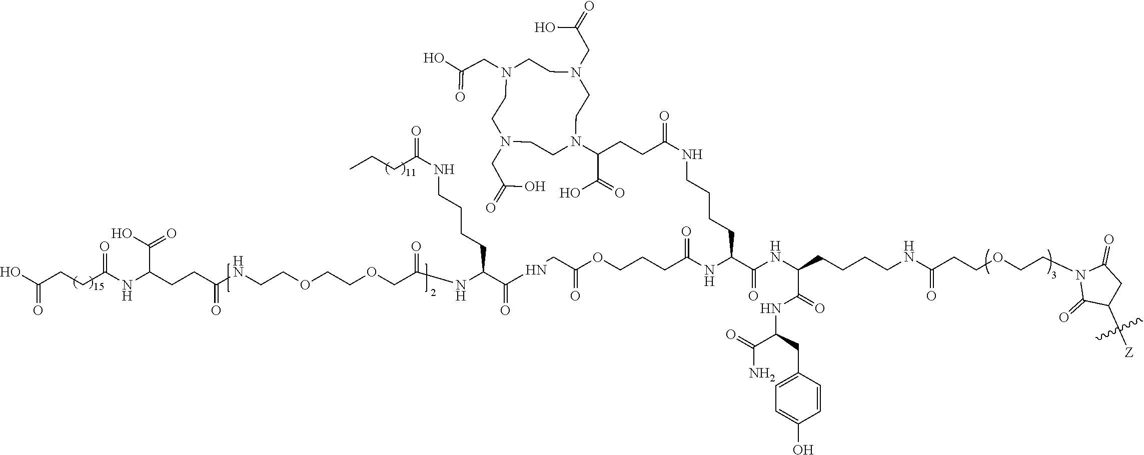 |
| C013 | [HO—C18-gGlu-OEG-OEG]2-Lys-ESL1-Lys(DOTAGA)-Lys(PEG3-Suc-Z)-Tyr-NH2 |
| | 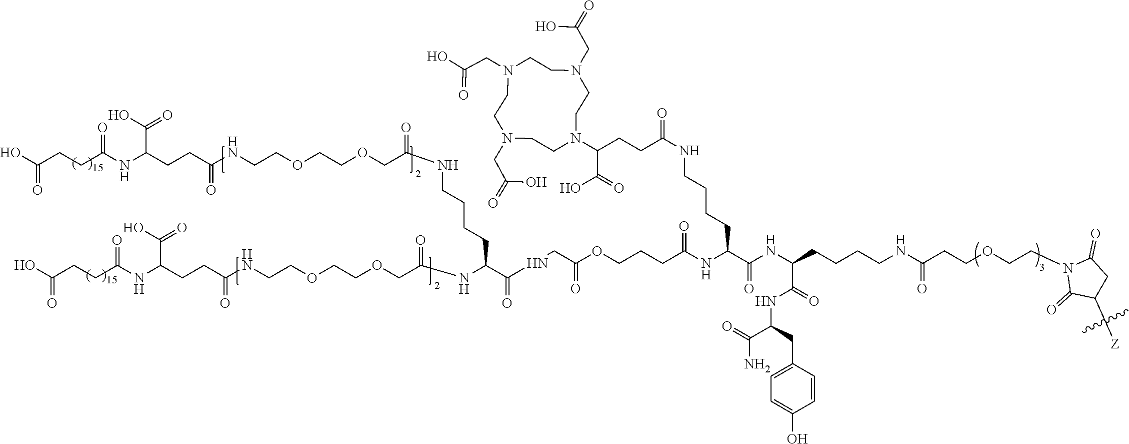 |
| C014 | HO—C18-gGlu-OEG-OEG-ESL1-Lys(SA-eLys-Val-Met-DOTAGA)-Lys(PEG3-Suc-Z)-Tyr-NH2 |
| | 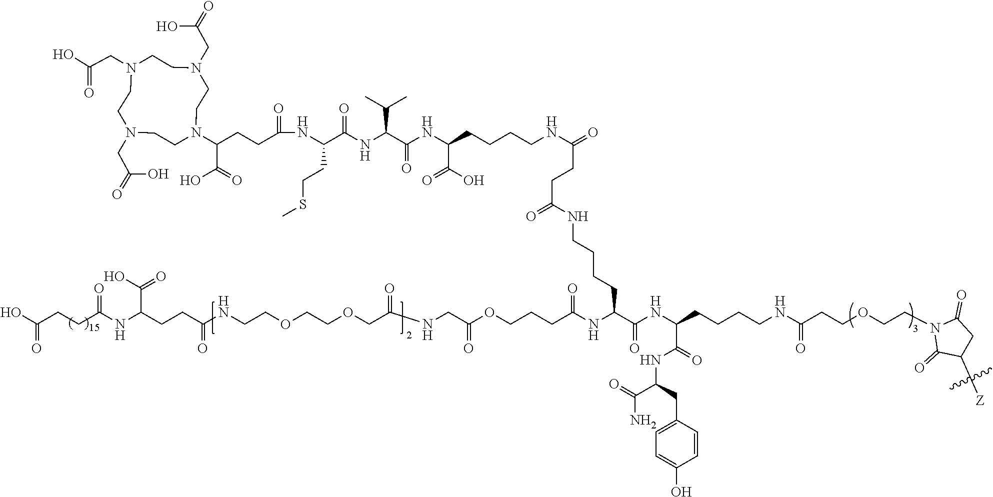 |
| C015 | [HO—C18-gGlu-OEG-OEG]2-Lys-ESL1-ESL1-Lys(DOTAGA)-Lys(COCH2—Z)-Tyr-NH2 |
| | 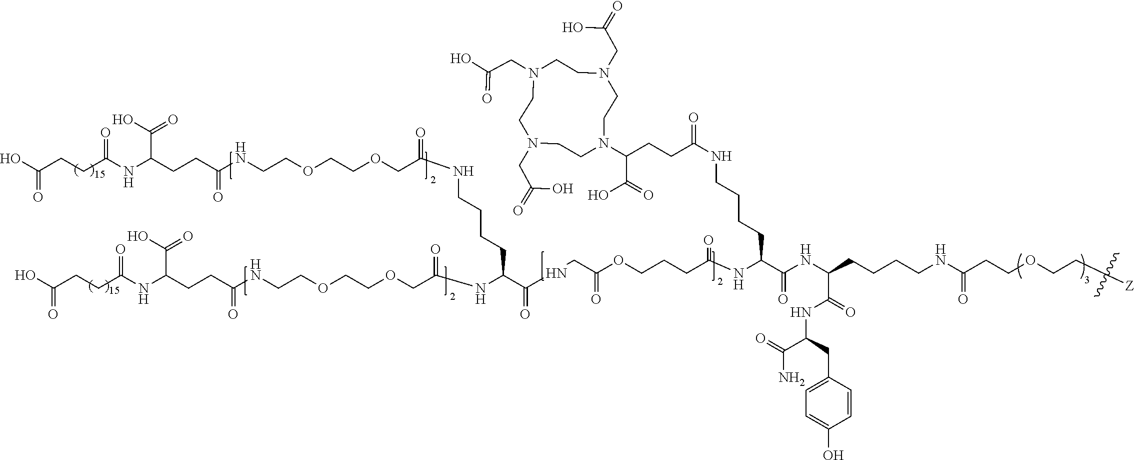 |
| C016 | HO—C18-gGlu-OEG-OEG-Lys(C14)-ESL1-Lys(DOTAGA)-Lys(COCH2—Z)-Tyr-NH2 |
| | 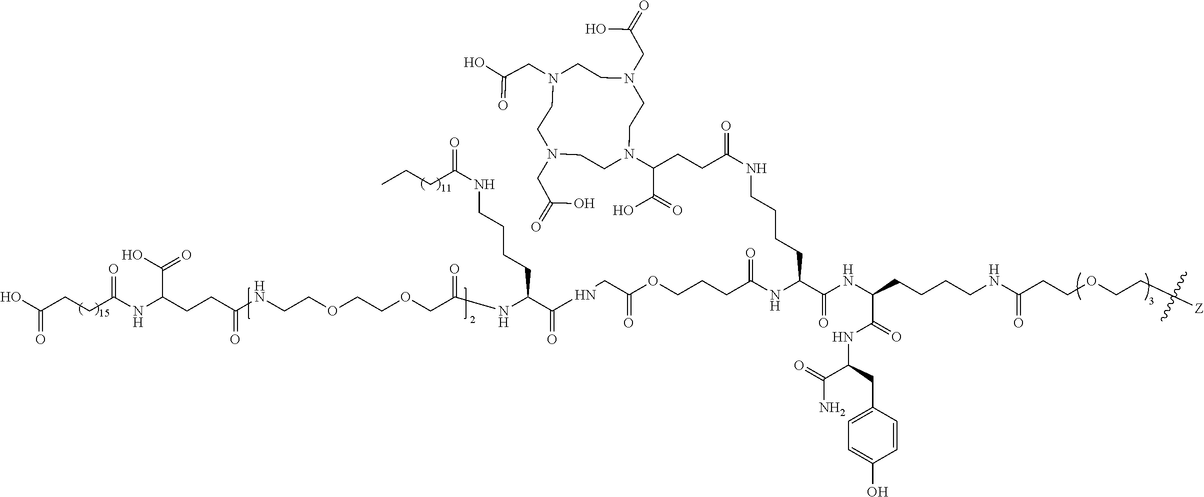 |
| C017 | HO—C18-gGlu-OEG-OEG-ESL1-Lys(DOTAGA)-Lys(COCH2—Z)-Tyr-NH2 |
| | 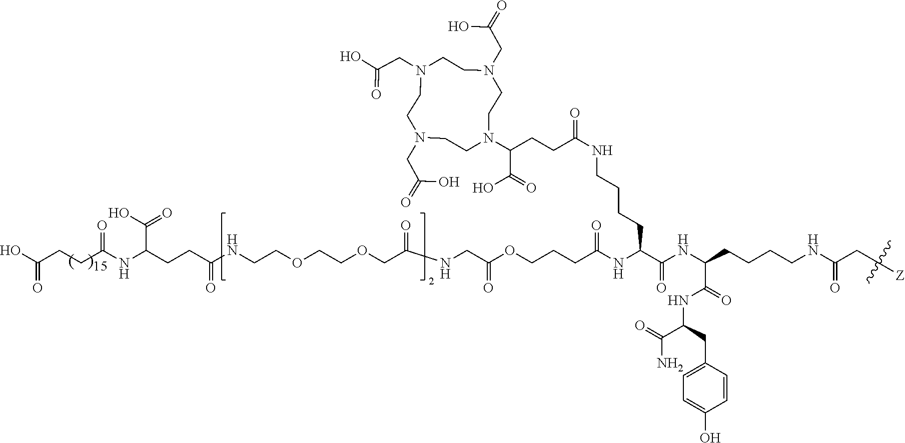 |
| C018 | HO—C18-gGlu-OEG-OEG-Lys(ESL1-DOTAGA)-Lys(COCH2—Z)-Tyr-NH2 |
| | 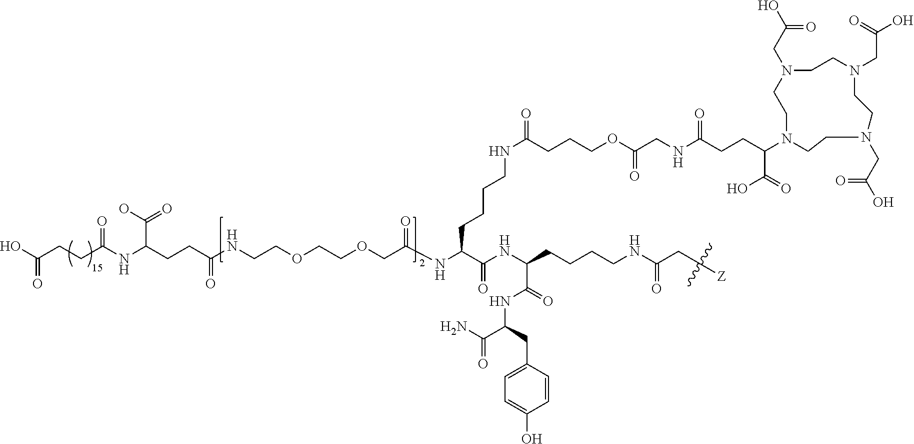 |
| C019 | HO-C18-gGlu-Ahx-ESL1-Lys(DOTAGA)-Lys(PEG3-Suc-Z)-Tyr-NH2 |
| | 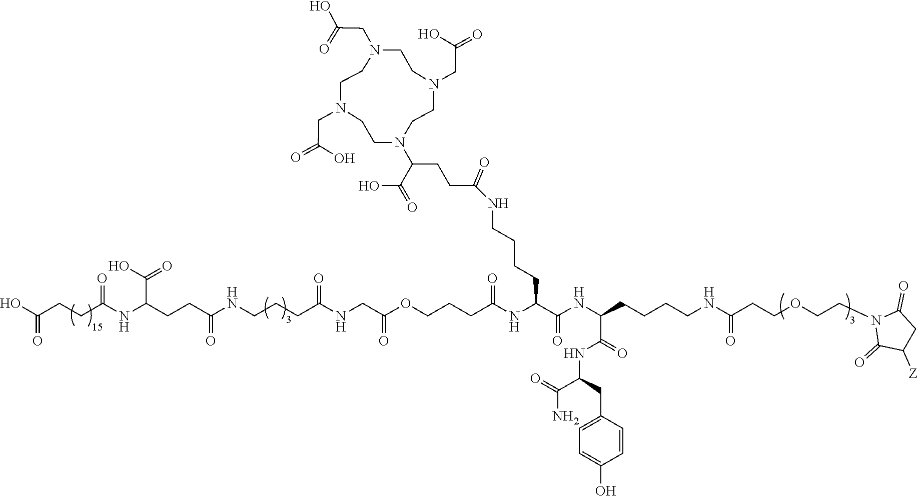 |
| C020 | HO-C18-gGlu-Ahx-Ahx-ESL1-Lys(DOTAGA)-Lys(PEG3-Suc-Z)-Tyr-NH2 |
| | 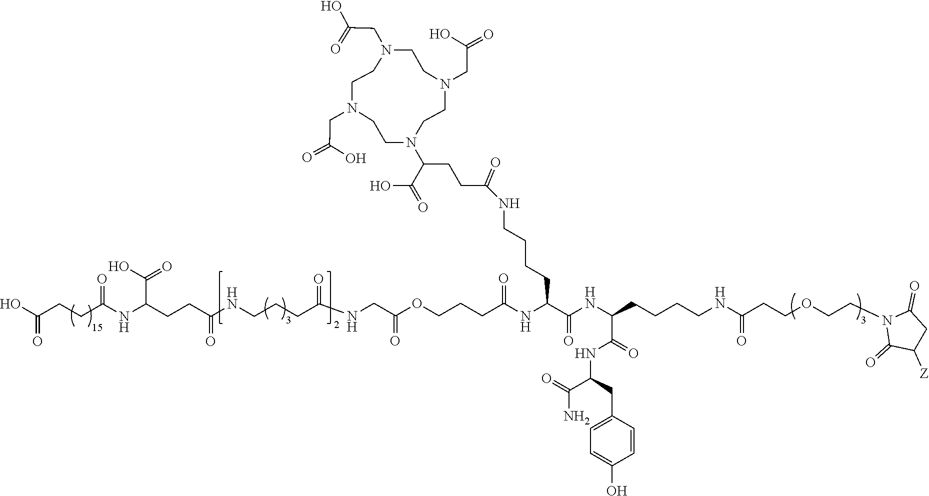 |
| C021 | [HO—C18-gGlu-OEG-OEG]2-Lys-Gly-6hHA-Lys(DOTAGA)-Lys(PEG3-Suc-Z)-Tyr-NH2 |
| | 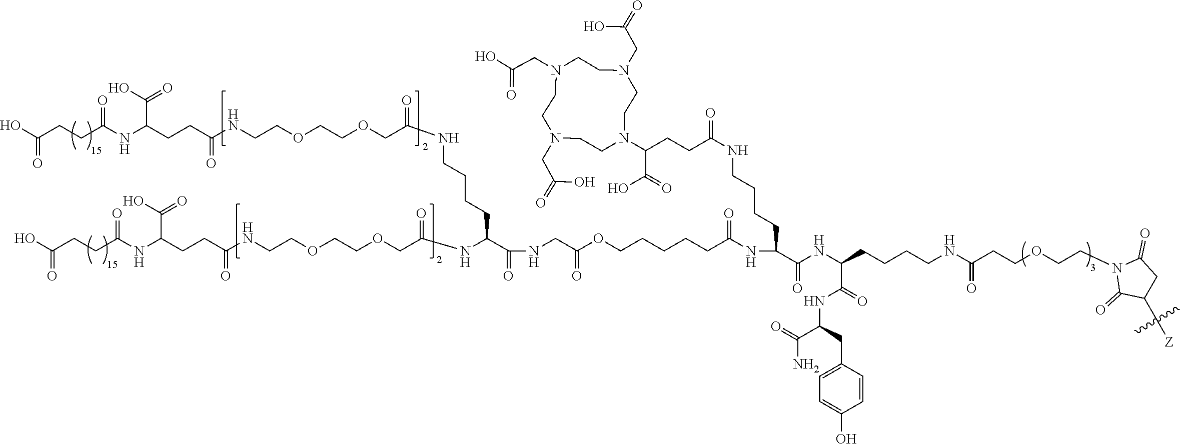 |
| C022 | [HO—C18-gGlu-OEG-OEG]2-Lys-Gly-5hPA-Lys(DOTAGA)-Lys(PEG3-Suc-Z)-Tyr-NH2 |
| | 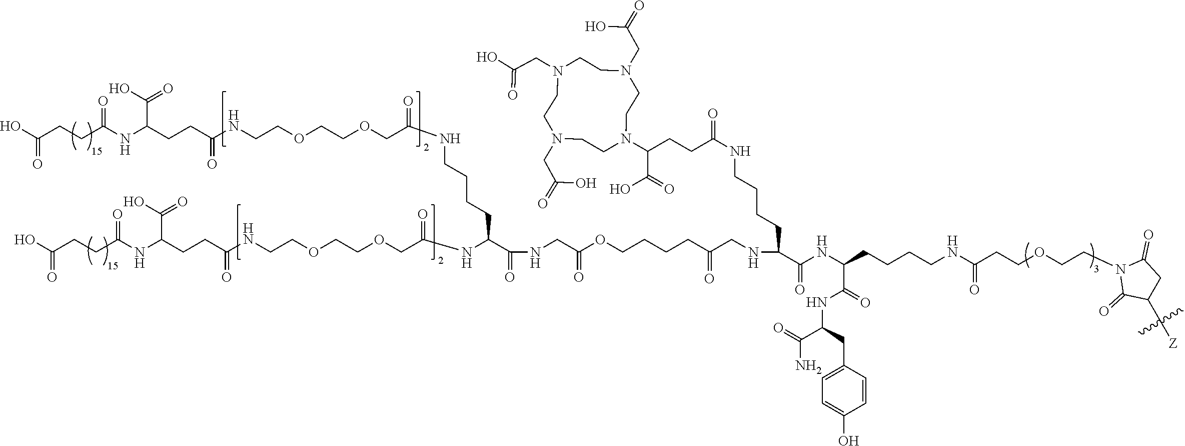 |
| C023 | HO—C18-gGlu-OEG-Lys(C14)-OEG-ESL1-Gly-Gly-Lys(DOTAGA)-Lys(PEG3-Suc-Z)-Tyr-NH2 |
| | 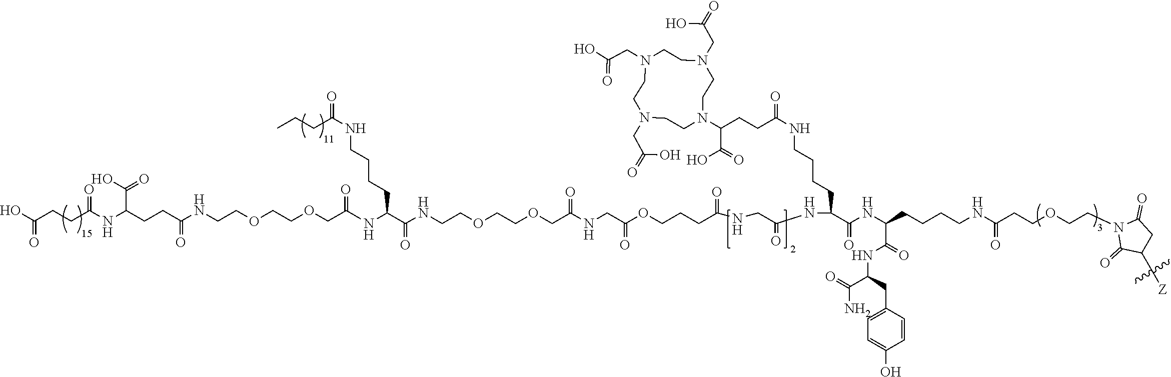 |
| C024 | HO—C18-gGlu-OEG-Lys(C14)-OEG-Gly-5hPA-Lys(DOTAGA)-Lys(PEG3-Suc-Z)-Tyr-NH2 |
| | 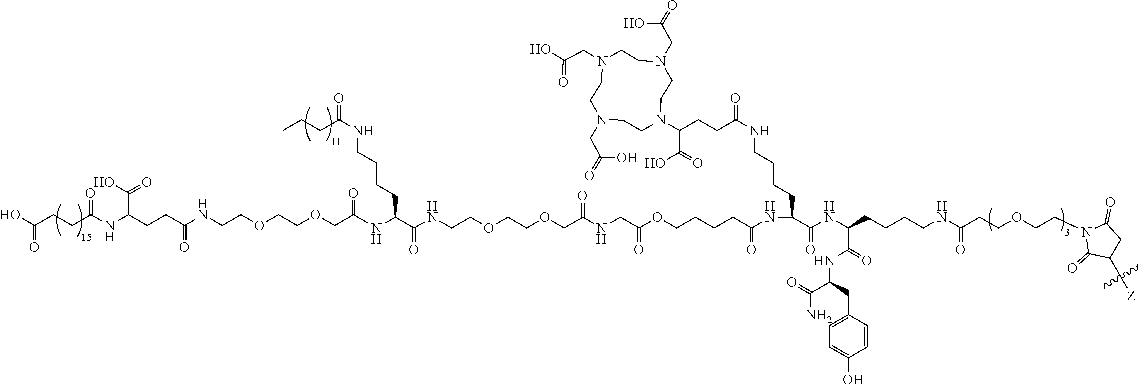 |
| C025 | HO—C18-gGlu-OEG-Lys(C14)-OEG-Gly-6hHa-Lys(DOTAGA)-Lys(PEG3-Suc-Z)-Tyr-NH2 |
| | 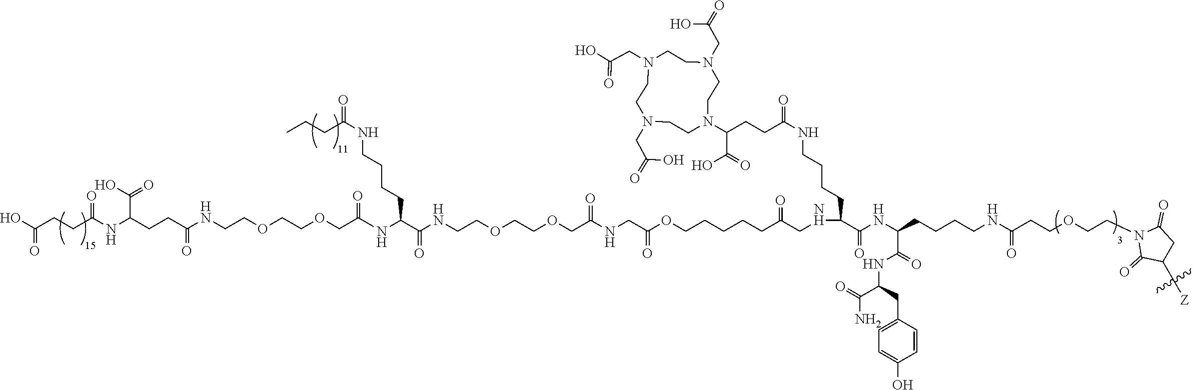 |
| C026 | [HO—C18-gGlu-OEG-OEG]2-Lys-ESL1-ESL1-Lys(DOTAGA)-Lys(PEG3-Suc-Z)-Tyr-NH2 |
| | 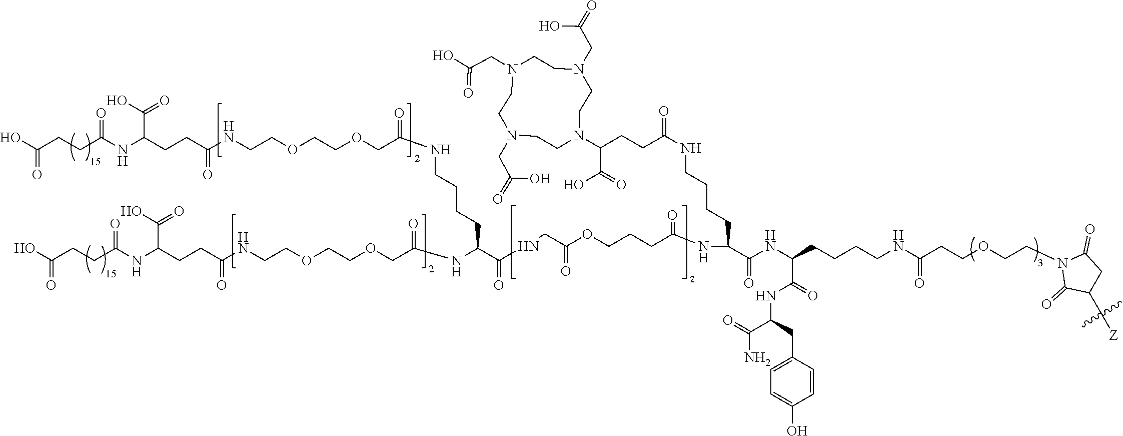 |
| C027 | [HO—C18-gGlu-OEG-OEG]2-Lys-ESL1-Gly-Lys(DOTAGA)-Lys(PEG-3-Suc-Z)-Tyr-NH2 |
| | 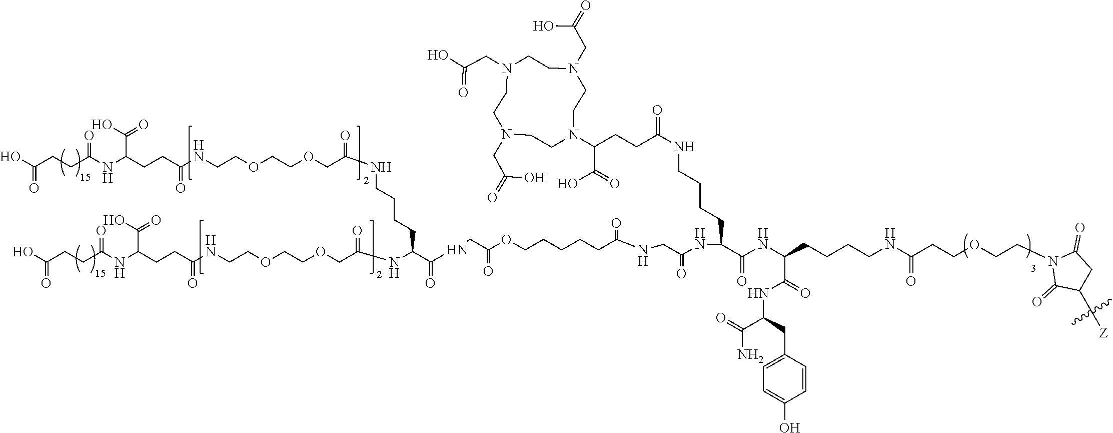 |
| C028 | [HO—C18-gGlu-OEG-OEG]2-Lys-ESL1-Gly-Gly-Lys(DOTAGA)-Lys(PEG3-Suc-Z)-Tyr-NH2 |
| | 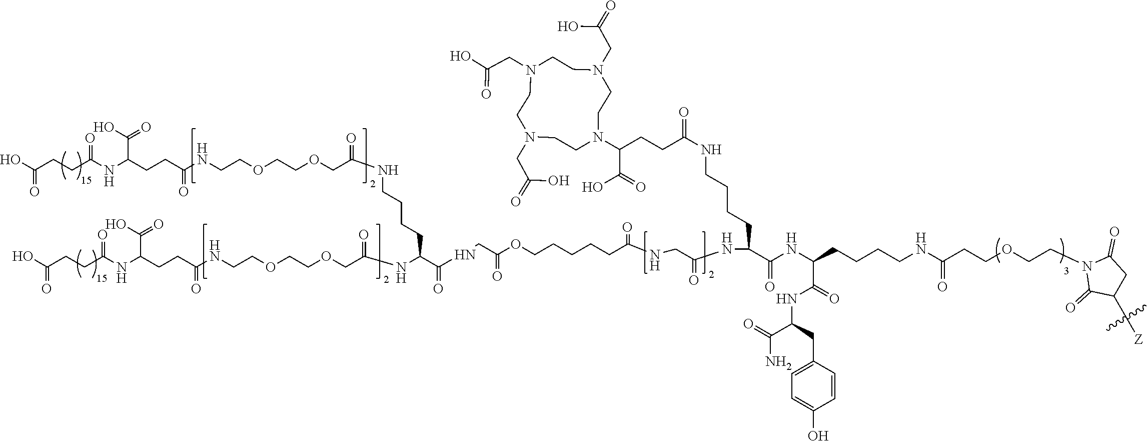 |
| C029 | [HO—C18-gGlu-PEG8-PEG8]2-Lys-ESL1-Lys(DOTAGA)-Lys(PEG3-Suc-Z)-Tyr-NH2 |
| | 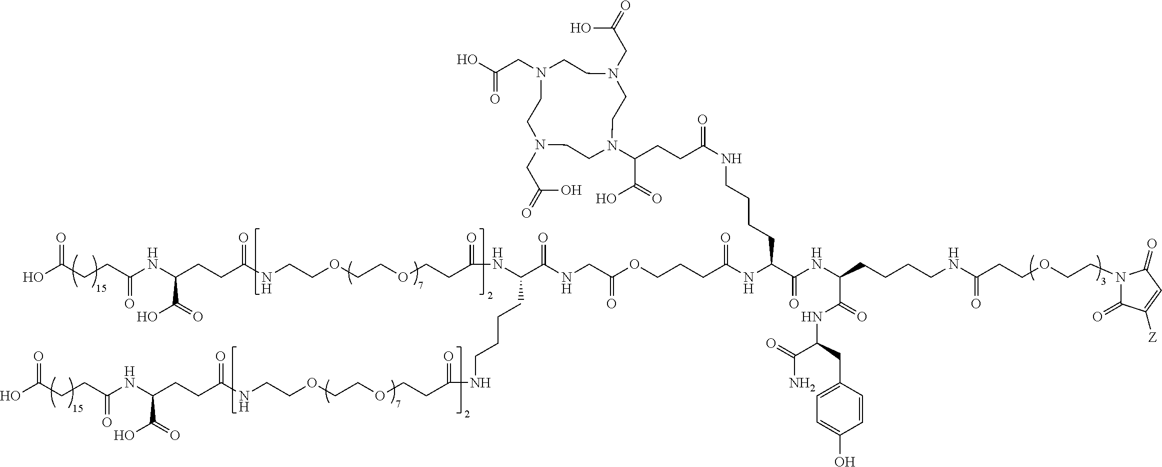 |
| C030 | [HO—C18-gGlu-gGlu-gGlu-OEG-OEG]2-Lys-ESL1-Lys(DOTAGA)-Lys(PEG3-Suc-Z)-Tyr-NH2 |
| | 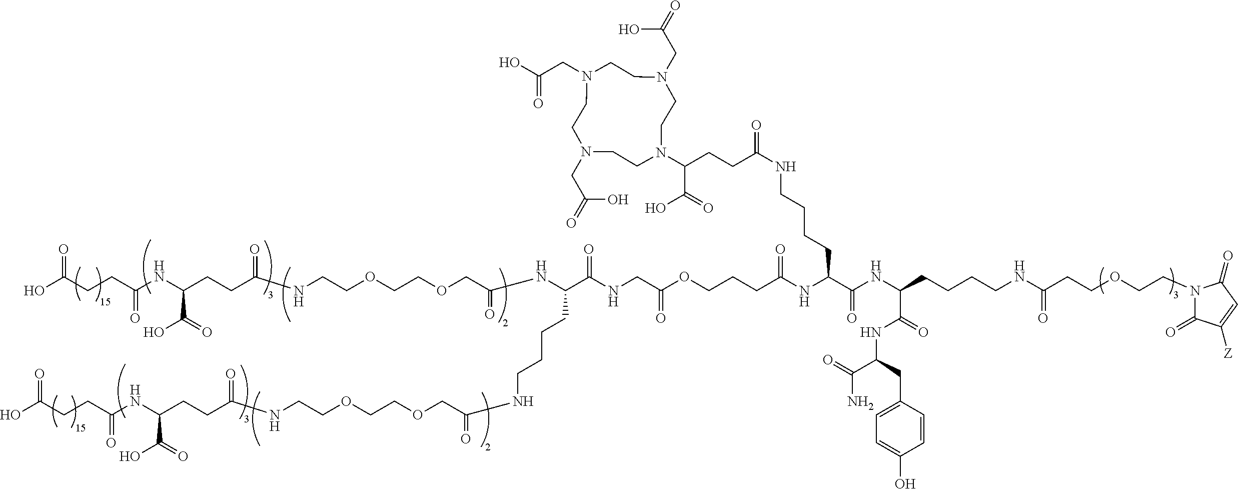 |
| C031 | [HO—C16-gGlu-OEG-OEG]2-Lys-ESL1-Lys(DOTAGA)-Lys(PEG3-Suc-Z)-Tyr-NH2 |
| | 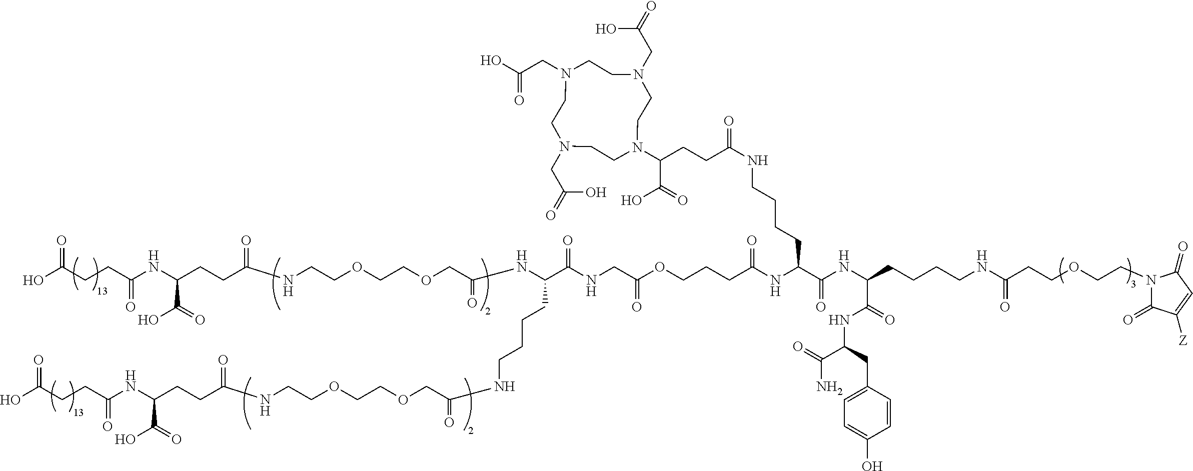 |
| C032 | [HO—C18-gGlu-PEG8-PEG8]2-Lys-Lys(ESL1-DOTAGA)-Lys(PEG3-Suc-Z)-Tyr-NH2 |
| | 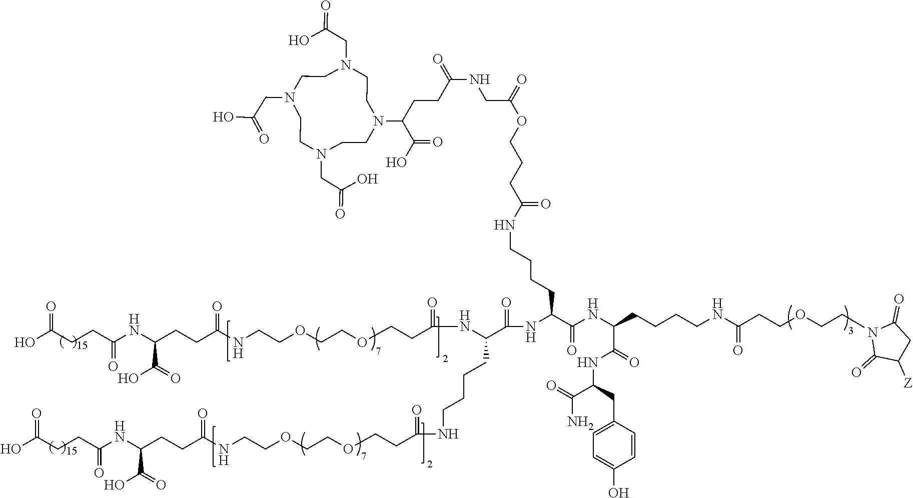 |
| C033 | [HO—C16-gGlu-PEG8-PEG8]2-Lys-Lys(ESL1-DOTAGA)-Lys(PEG3-Suc-Z)-Tyr-NH2 |
| | 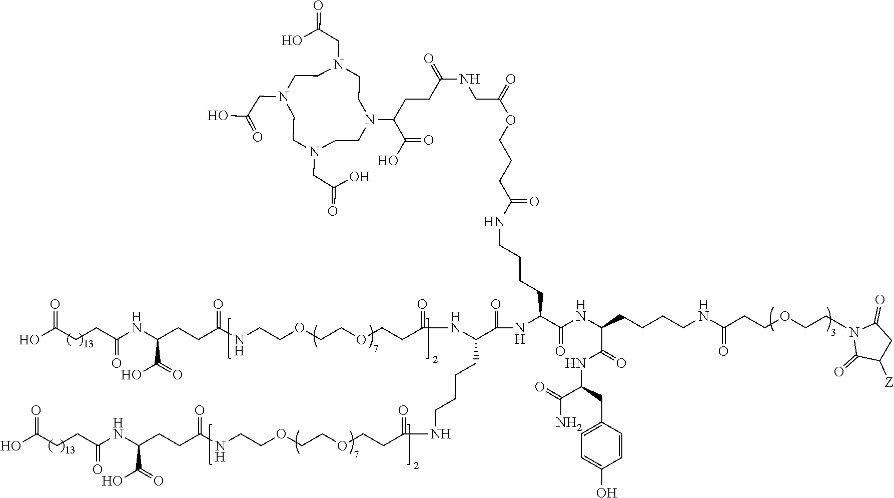 |
| C034 | [HO—C14-gGlu-PEG8-PEG8]2-Lys-Lys(ESL1-DOTAGA)-Lys(PEG-Suc-Z)-Tyr-NH2 |
| | 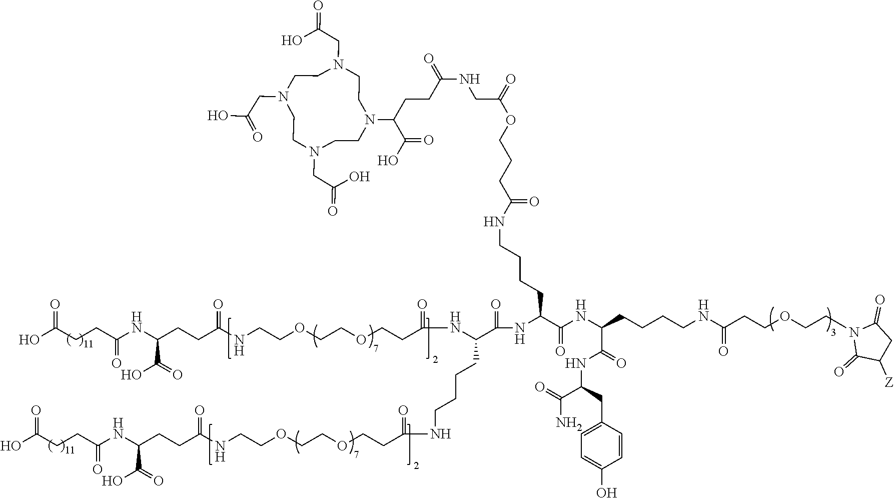 |
| C035 | [HO—C18-gGlu-PEG8-PEG8]2-Lys-Lys(ESL1-ESL1-DOTAGA)-Lys(PEG3-Suc-Z)-Tyr-NH2 |
| | 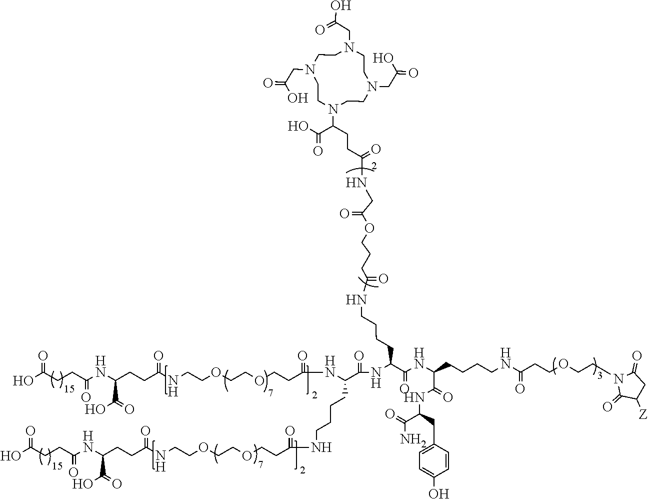 |
| C036 | [HO—C18-gGlu-PEG8-PEG8-PEG8-PEG8]2-Lys-Lys(ESL1-DOTAGA)-Lys(PEG3-Suc-Z)-Tyr-NH2 |
| | 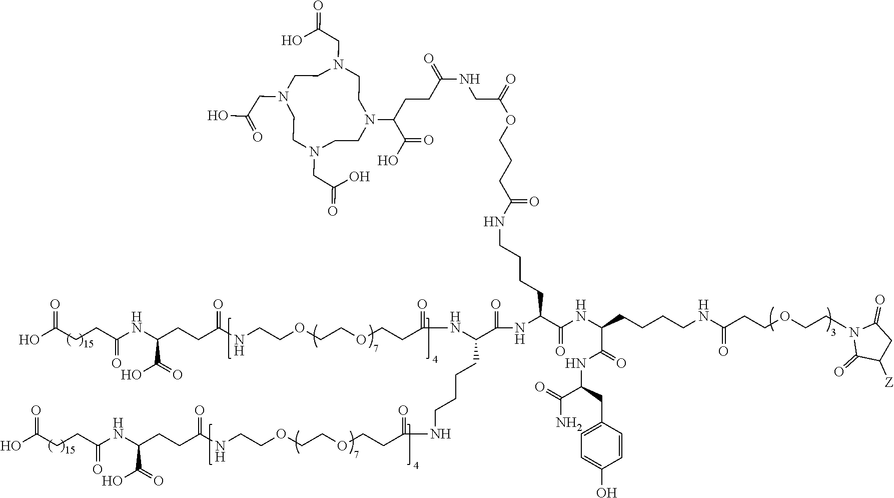 |
| C037 | HO—C18-gGlu-PEG8-PEG8-Lys(C12)-Glu-PEG8-PEG8-Lys(ESL1-DOTAGA)-Lys(PEG3-Suc-Z)-Tyr-NH2 |
| | 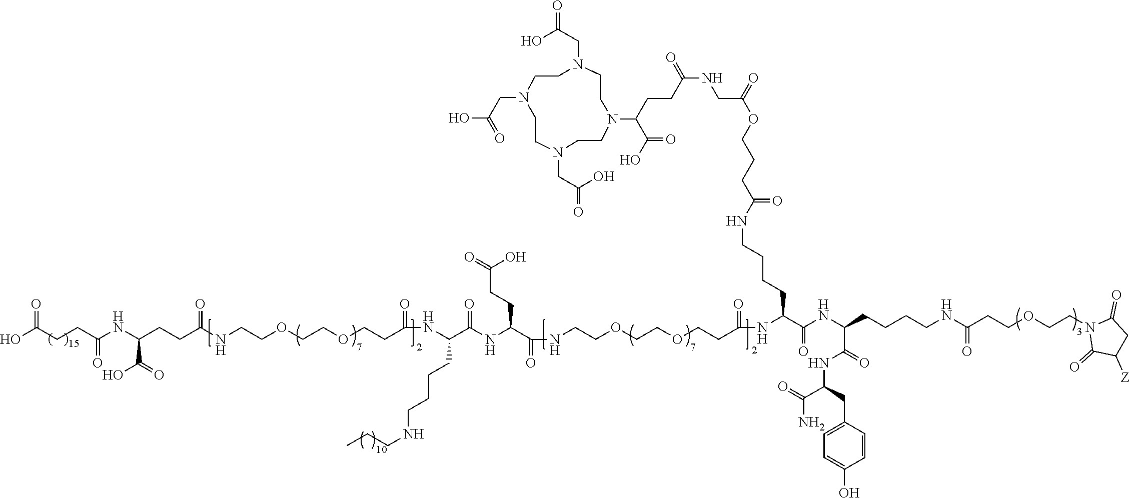 |
| C038 | HO—C18-gGlu-PEG8-PEG8-Lys(C12—OH)-Glu-PEG8-PEG8-Lys(ESL1-DOTAGA)-Lys(PEG3-Suc-Z)-Tyr-NH2 |
| | 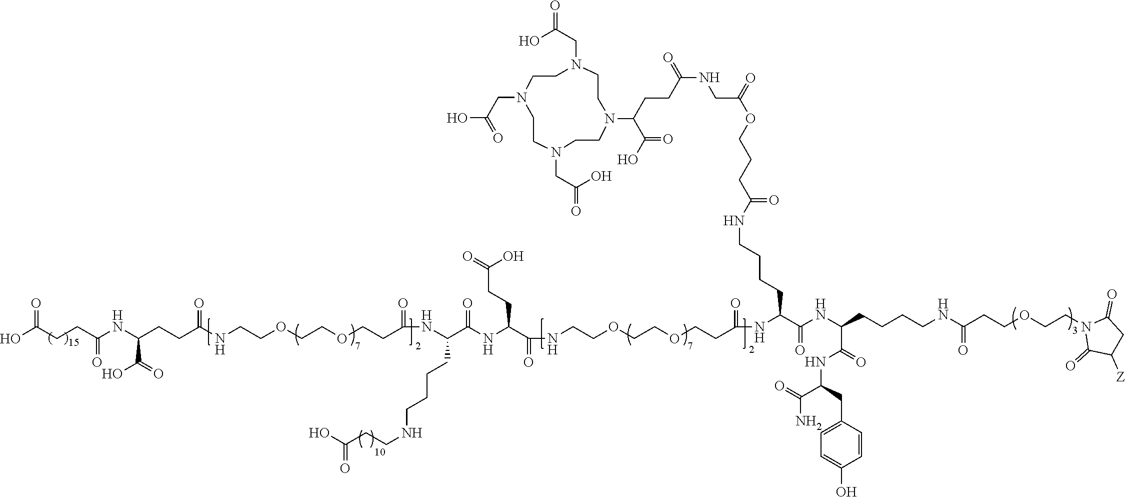 |
| C039 | HO—C18-gGlu-PEG8-NAl-PEG8-Nal-PEG8-Lys(ESL1-DOTAGA)-Lys(PEG3-Suc-Z)-Tyr-NH2 |
| | 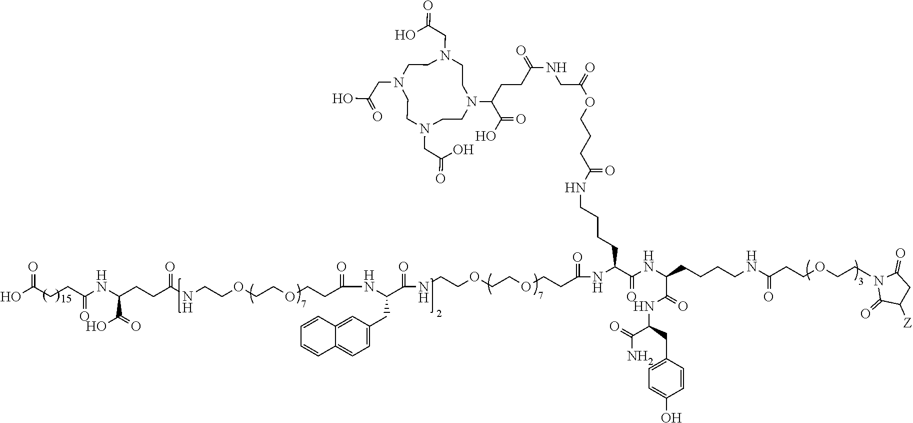 |
| C040 | HO—C18-gGlu-PEG8-PEG8-Lys(HO—C16)-Glu-PEG8-PEG8-Lys(ESL1-DOTAGA)-Lys(PEG3-Suc-Z)-Tyr-NH2 |
| | 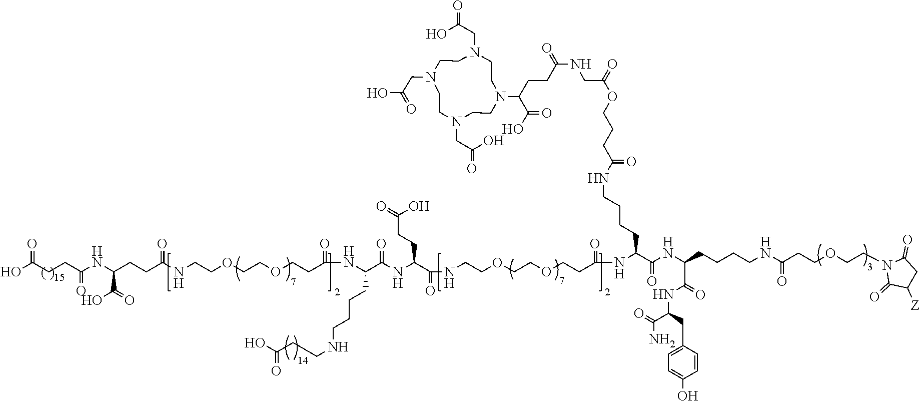 |
| C041 | [HO—C18-gGlu-PEG8-PEG8-PEG8-PEG8]2-Lys-Lys(OEG-ESL1-OEG-DOTAGA)-Lys(PEG3-Suc-Z)-Tyr-NH2 |
| | 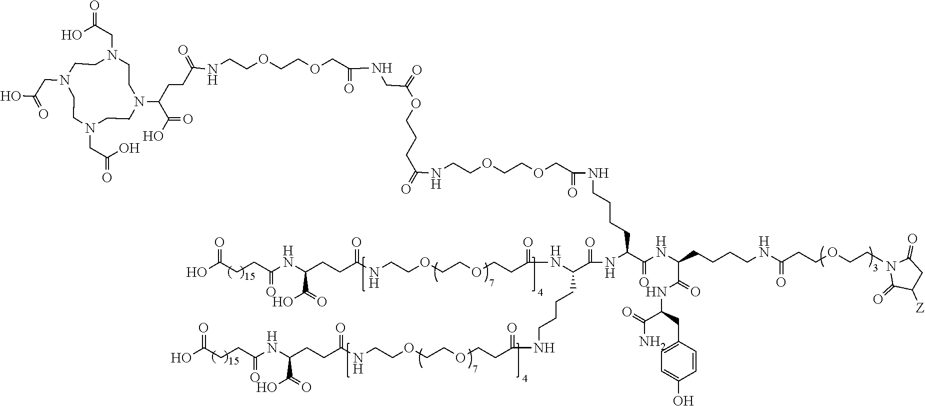 |
| C042 | HO—C18-gGlu-PEG12-PEG12-Glu-Lys(C16)-Glu-PEG12-PEG12-Lys(ESL1-DOTAGA)-Lys(PEG3-Suc-Z)-Tyr-NH2 |
| | 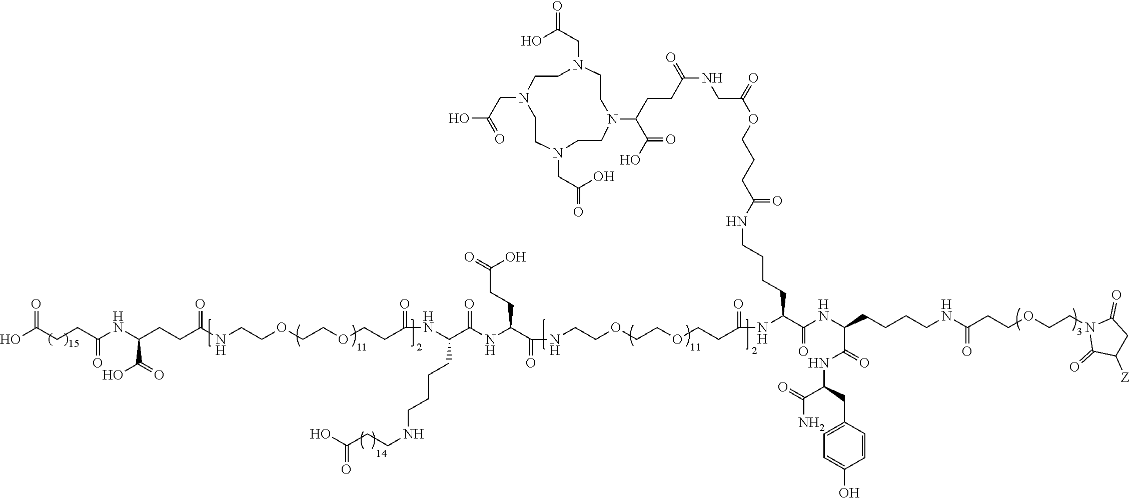 |
| C043 | [HO—C17-gGlu-PEG8-PEG8-PEG8-PEG8]2-Lys-Lys(ESL1-DOTAGA)-Lys(PEG3-Suc-Z)-Tyr-NH2 |
| | 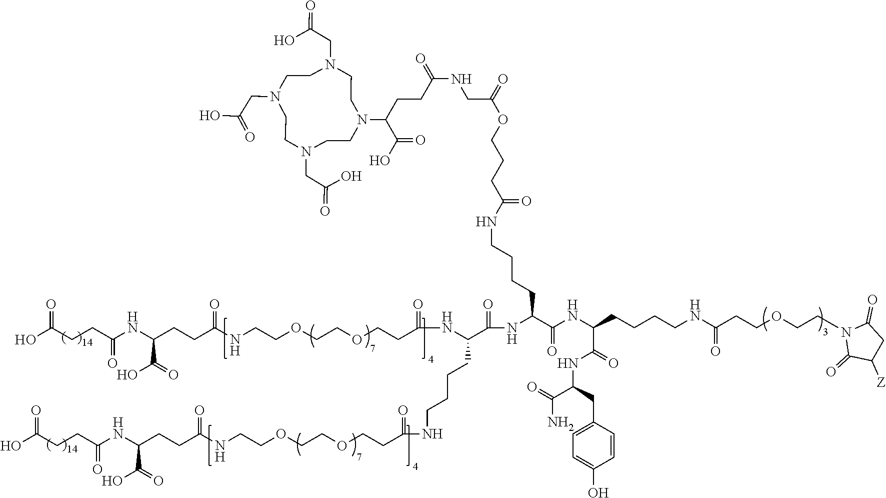 |
| C044 | HO—C20-gGlu-PEG12-PEG12-PEG12-PEG12-PEG12-PEG12-Lys(ESL1-DOTAGA)-Lys(PEG3-Suc-Z)-Tyr-NH2 |
| | 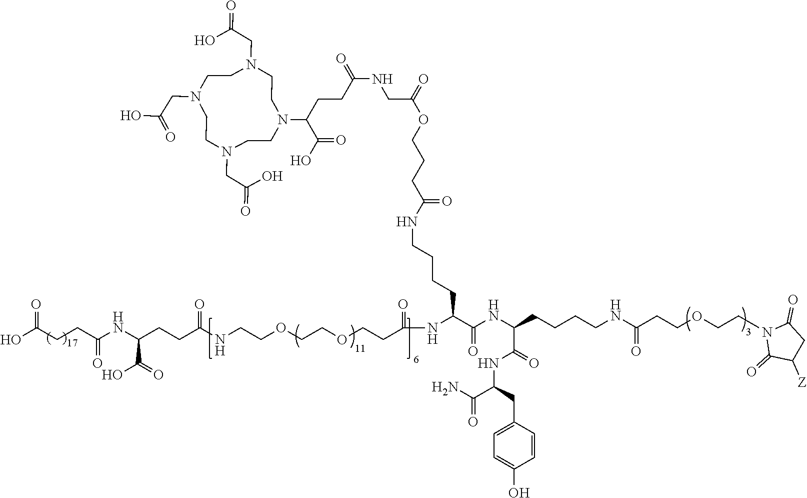 |
| C045 | [HO—C18-gGlu]2-Lys-PEG12-PEG12-PEG12-PEG12-PEG12-PEG12-Lys(ESL1-DOTAGA)-Lys(PEG3-Suc-Z)-Tyr-NH2 |
| | 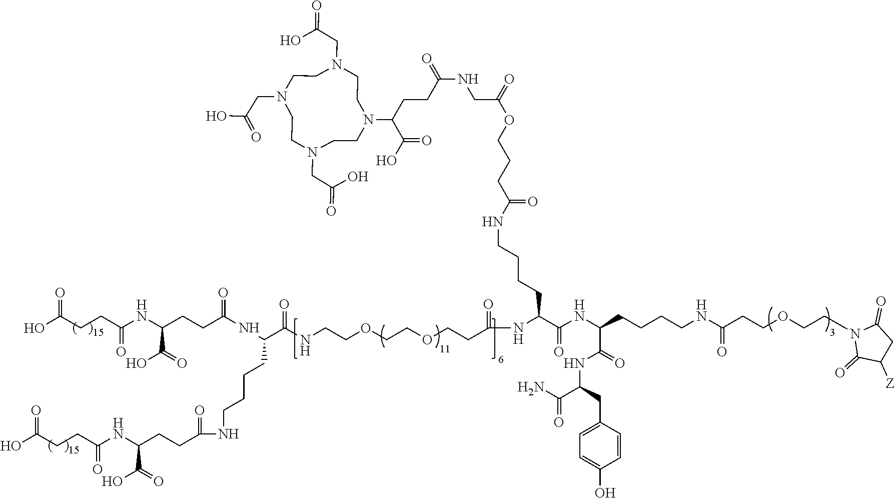 |
| C046 | [HO—C18-gGlu-PEG8-PEG8-PEG8-PEG8]2-Lys-Lys(OEG-OEG-ESL1-OEG-OEG-DOTAGA)-Lys(PEG3-Suc-Z)-Tyr-NH2 |
| | 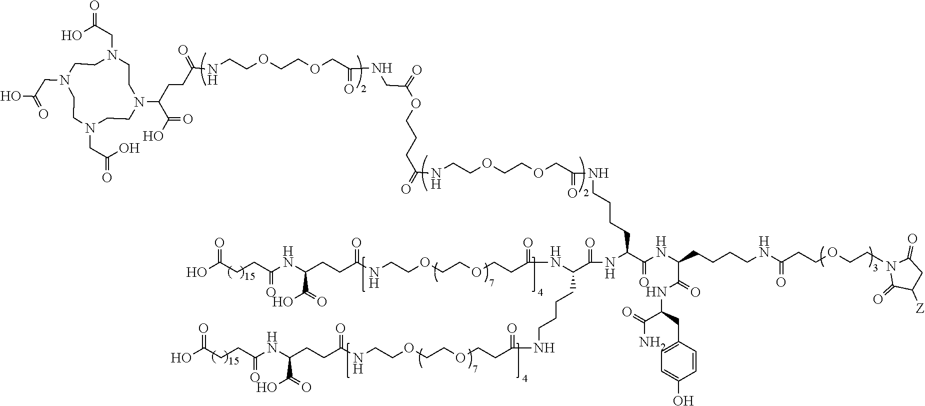 |
| C047 | [HO—C18-gGlu-PEG8-PEG8-PEG8-PEG8]2-Lys-Lys(DOTAGA)-Lys(PEG3-Suc-Z)-Tyr-NH2 |
| | 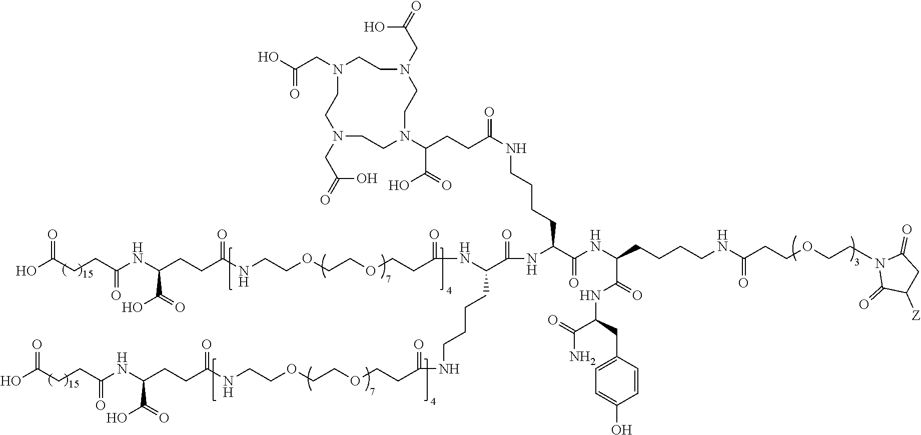 |
| C048 | HO—C20-gGlu-PEG8-PEG8-PEG8-PEG8-Lys(DOTAGA)-Lys(PEG3-Suc-Z)-Tyr-NH2 |
| | 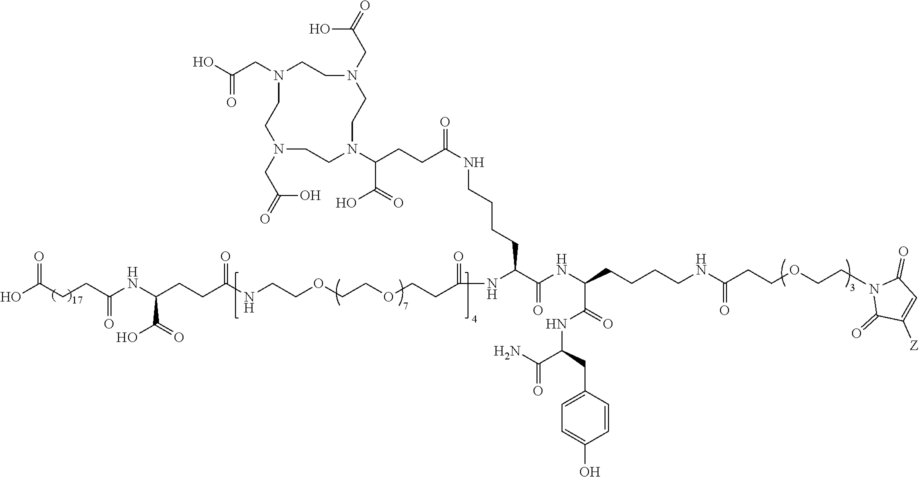 |
| C049 | HO—C18-gGlu-PEG8-PEG8-PEG8-PEG8-Lys(DOTAGA)-Lys(PEG3-Suc-Z)-Tyr-NH2 |
| | 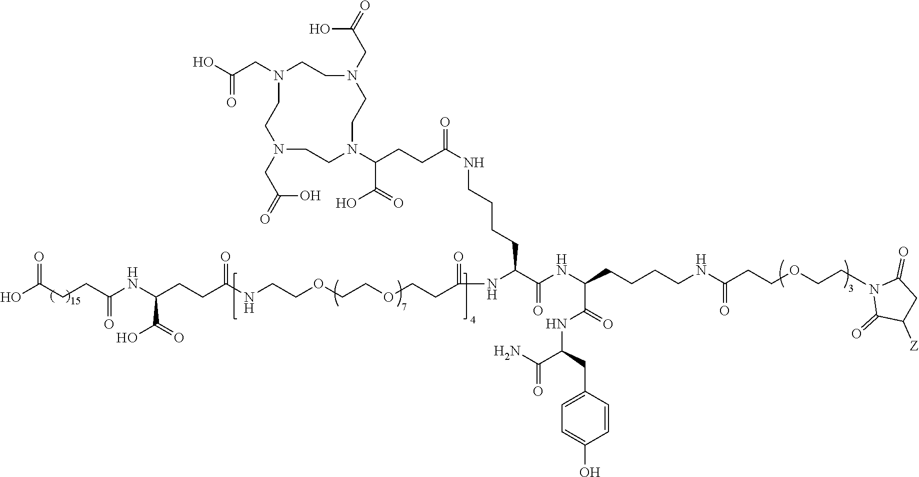 |
| C050 | HO—C18-gGlu-PEG8-PEG8-PEG8-PEG8-Lys(4hPA-Gly-DOTAGA)-Lys(PEG3-Suc-Z)-Tyr-NH2 |
| | 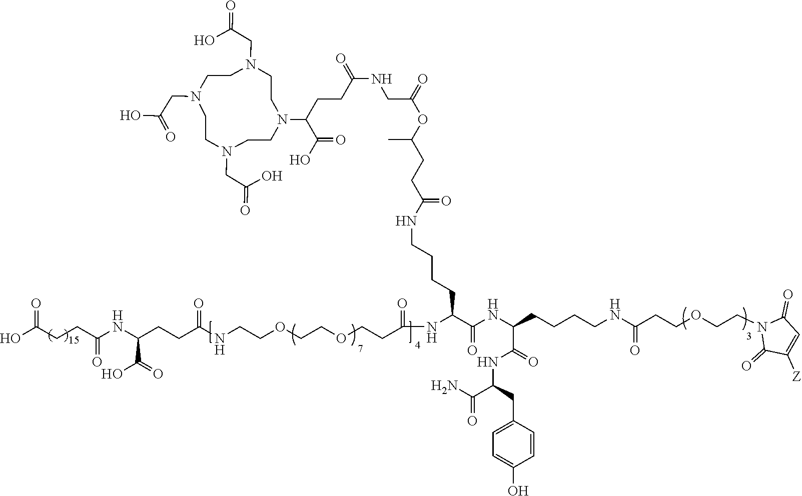 |
| C051 | HO—C18-gGlu-PEG8-PEG8-PEG8-PEG8-Lys(4hBA-Leu-DOTAGA)-Lys(PEG3-Suc-Z)-Tyr-NH2 |
| | 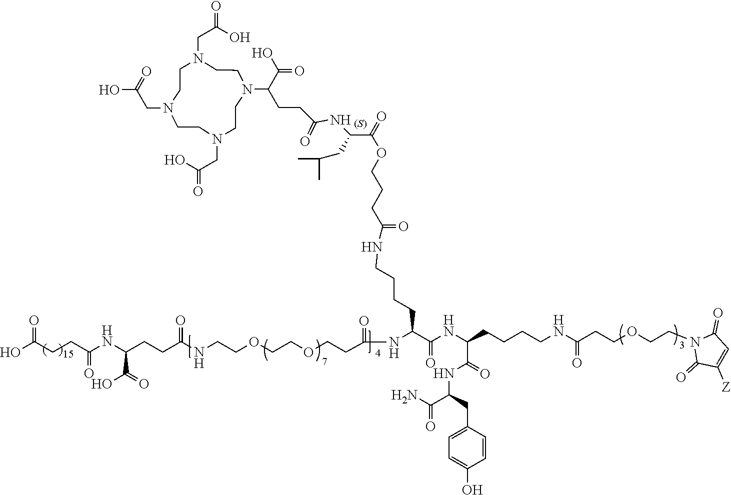 |
| C052 | HO—C18-gGlu-PEG8-PEG8-PEG8-PEG8-Lys(4hPA-Leu-DOTAGA)-Lys(PEG3-Suc-Z)-Tyr-NH2 |
| | 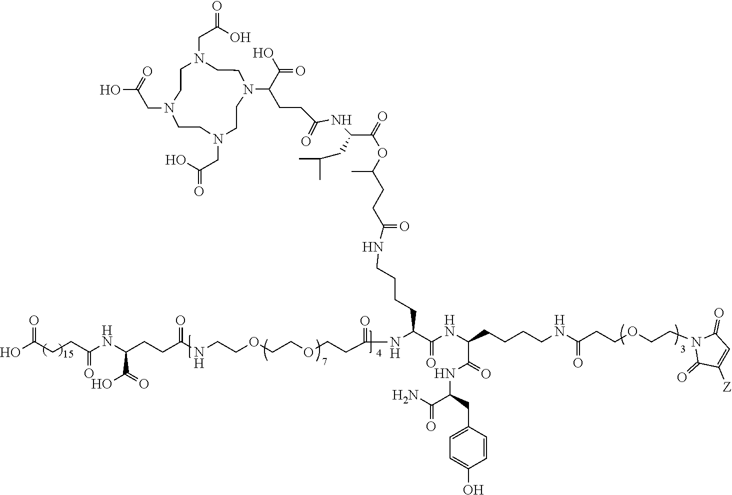 |
| C053 | HO—C18-gGlu-PEG8-PEG8-PEG8-PEG8-Gly-4hPA-Lys(DOTAGA)-Lys(PEG3-Suc-Z)-Tyr-NH2 |
| | 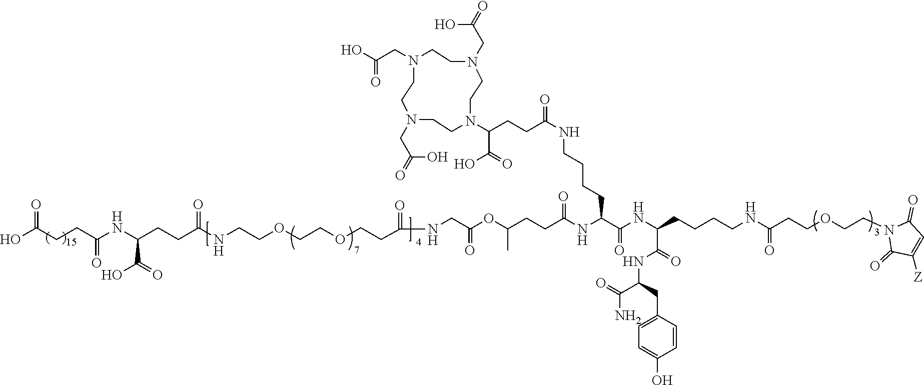 |
| C054 | HO—C18-gGlu-PEG8-PEG8-PEG8-PEG8-Leu-4hBA-Lys(DOTAGA)-Lys(PEG3-Suc-Z)-Tyr-NH2 |
| | 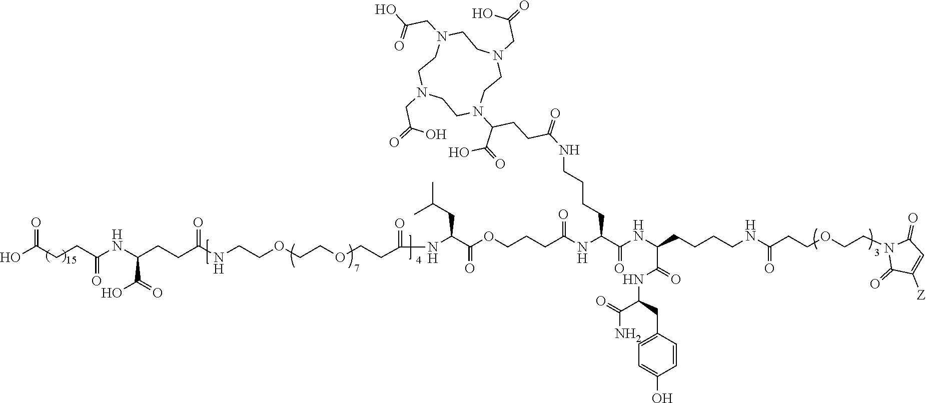 |
| C055 | HO—C18-gGlu-PEG8-PEG8-PEG8-PEG8-Leu-4hPA-Lys(DOTAGA)-Lys(PEG3-Suc-Z)-Tyr-NH2 |
| | 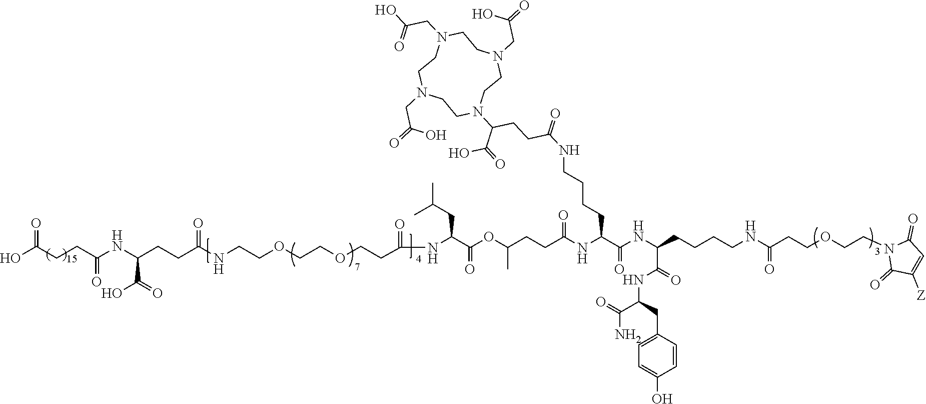 |
| C056 | HO—C18-gGlu-PEG8-PEG8-PEG8-PEG8-Gly-5hPA-Lys(DOTAGA)-Lys(PEG3-Suc-Z)-Tyr-NH2 |
| | 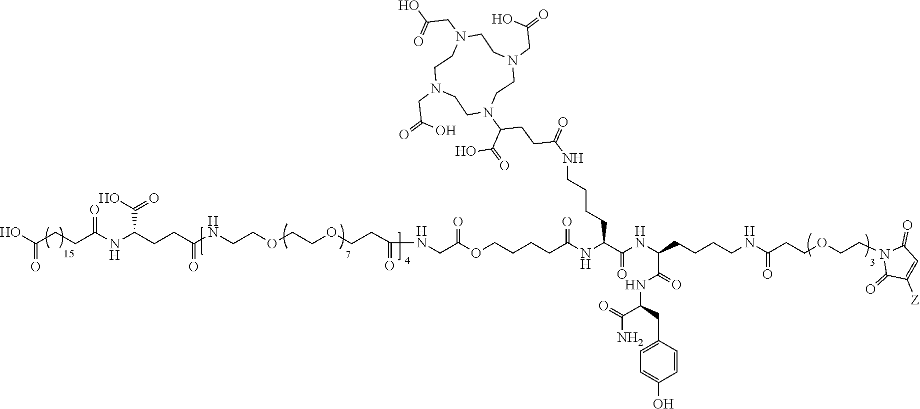 |
| C057 | HO—C18-gGlu-PEG8-PEG8-PEG8-PEG8-Gly-6hHA-Lys(DOTAGA)-Lys(PEG3-Suc-Z)-Tyr-NH2 |
| | 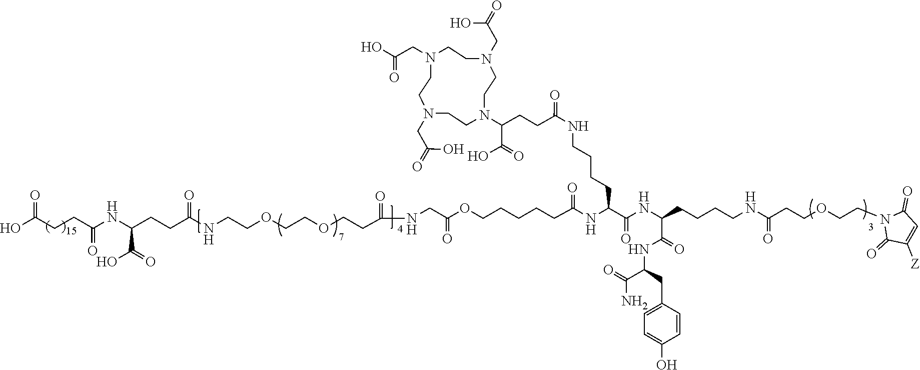 |
| C058 | HO—C18-gGlu-PEG8-PEG8-PEG8-PEG8-Lys(5hPA-Gly-DOTAGA)-Lys(PEG3-Suc-Z)-Tyr-NH2 |
| | 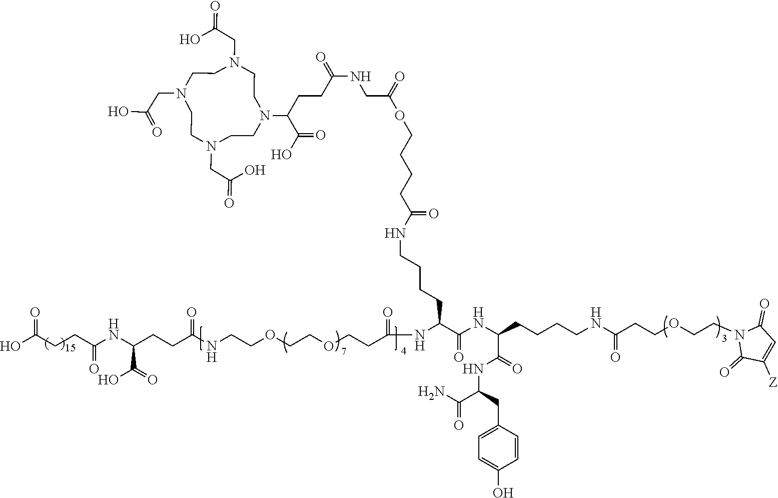 |
| C059 | HO—C18-gGlu-PEG8-PEG8-PEG8-PEG8-Lys(6hPA-Gly-DOTAGA)-Lys(PEG3-Suc-Z)-Tyr-NH2 |
| | 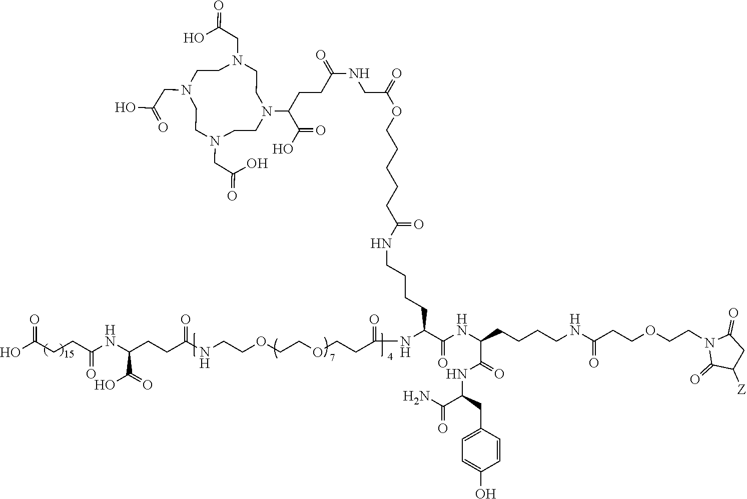 |
| C060 | [CO—C18-gGlu-PEG8-PEG8-PEG8-PEG8]2-Lys-Lys(PEG8-ESL1-PEG8-DOTAGA)-Lys(PEG3-Suz-Z)-Tyr-NH2 |
| | 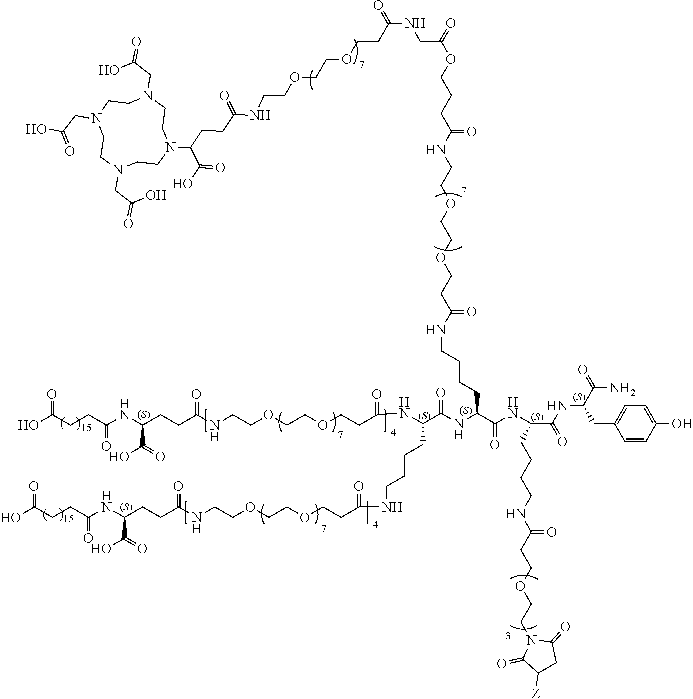 |
| C061 | HO—C20-gGlu-PEG8-PEG8-PEG8-PEG8-Lys(PEG8-ESL1-PEG8-DOTAGA)-Lys(PEG3-Suc-Z)-Tyr-NH2 |
| | 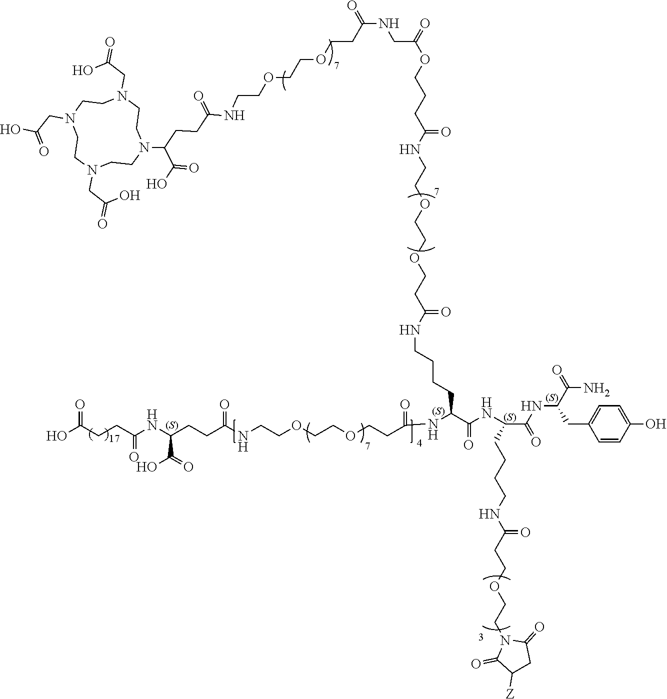 |
| C062 | HO—C18-gGlu-PEG8-PEG8-PEG8-PEG8-Lys(ESL1-DOTAGA)-Lys(PEG3-Suc-Z)-Tyr-NH |
| | 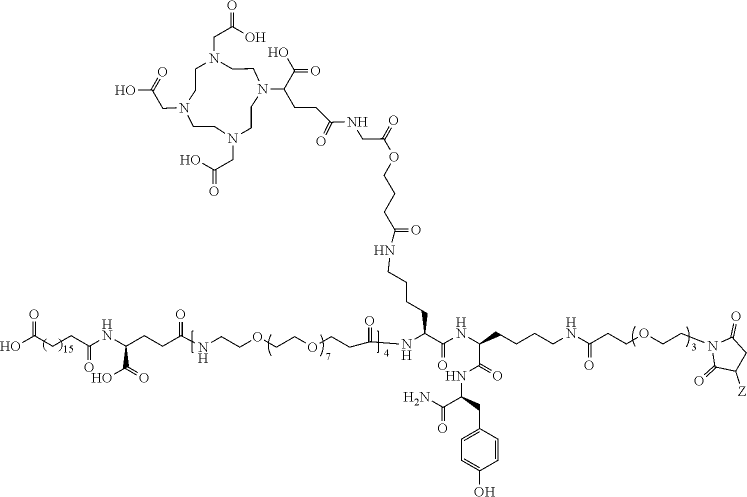 |
| C063 | HO—C20-gGlu-PEG8-PEG8-PEG8-PEG8-ESL1-Lys(DOTAGA)-Lys(PEG3-Suc-Z)-Tyr-NH |
| | 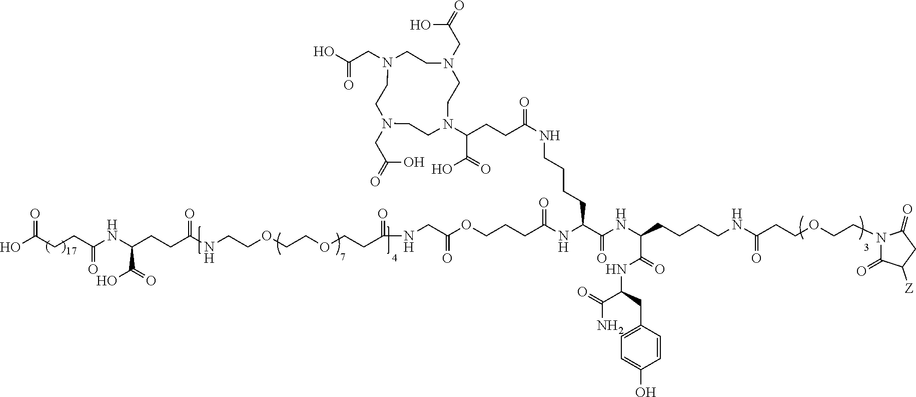 |
| C064 | HO—C20-gGlu-PEG8-PEG8-PEG8-PEG8-Lys(ESL1-DOTAGA)-Lys(PEG3-Suc-Z)-Tyr-NH2 |
| | 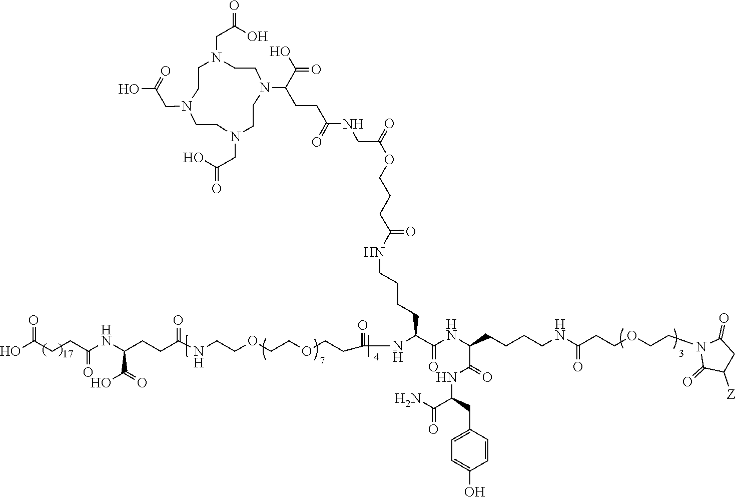 |
| C065 | HO—C20-gGlu-PEG8-PEG8-PEG8-PEG8-Lys(PEG8-ESL1-PEG8-DOTAGA)-Lys(PEG3-Suc-Z)-Tyr-NH2 |
| | 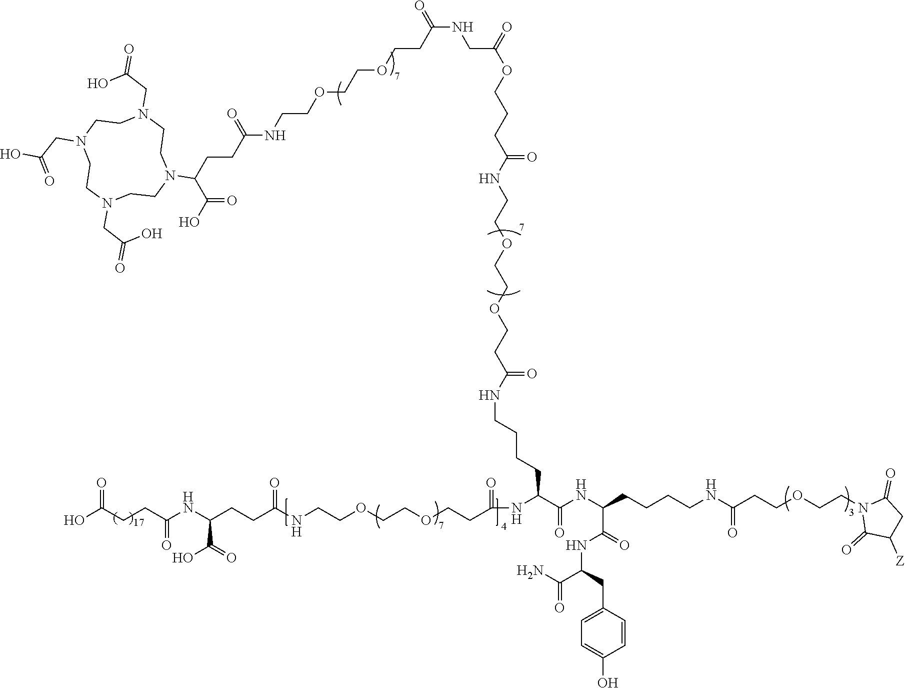 |
| C066 | HO—C20-gGlu-PEG8-PEG8-PEG8-PEG8-Lys(5hPA-Gly-DOTAGA)-Lys(PEG3-Suc-Z)-Tyr-NH2 |
| | 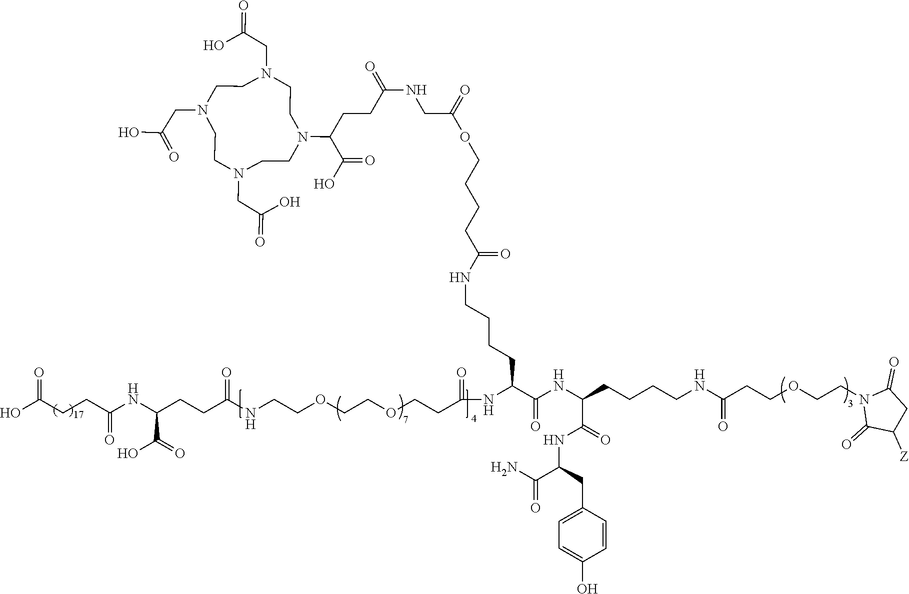 |
| C067 | HO—C20-gGlu-PEG8-PEG8-PEG8-PEG8-Lys(6hHA-Gly-DOTAGA)-Lys(PEG3-Suc-Z)-Tyr-NH2 |
| | 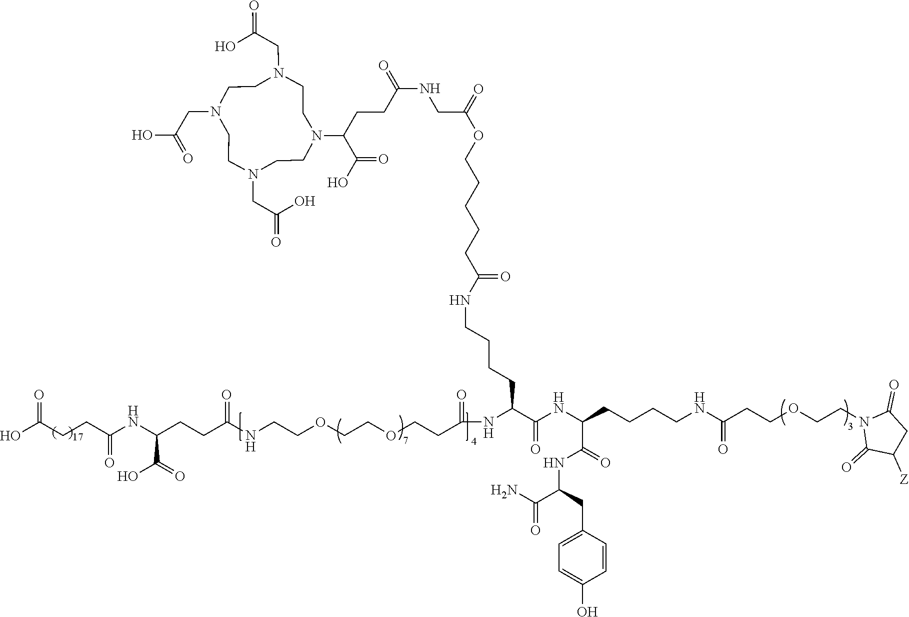 |
| C068 | [HO—C18-gGlu-PEG8-PEG8-PEG8-PEG8]2-Lys-Lys(PEG8-OEG-PEG8-DOTAGA)-Lys(PEG3-Suc-Z)-Tyr-NH2 |
| | 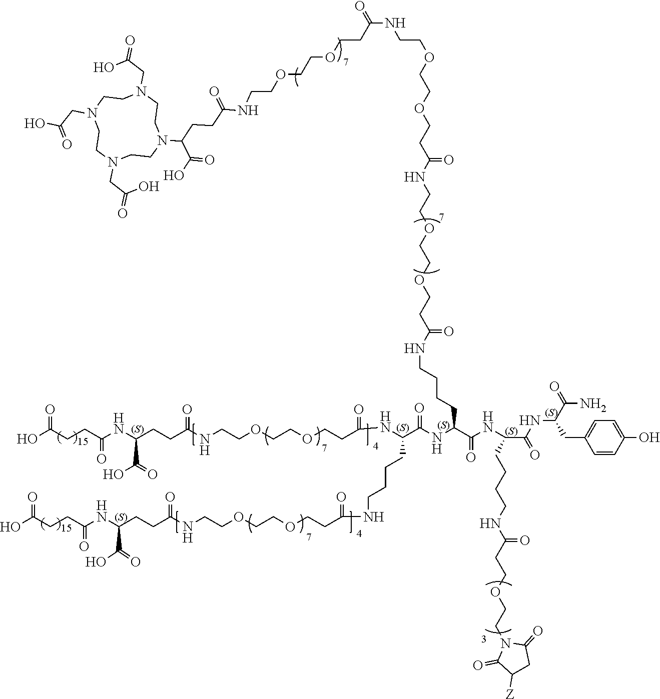 |
| C069 | HO—C20-gGlu-PEG8-PEG8-PEG8-PEG8-Lys(PEG6-ESL1-DOTAGA)-Lys(PEG3-Suc-Z)-Tyr-NH2 |
| | 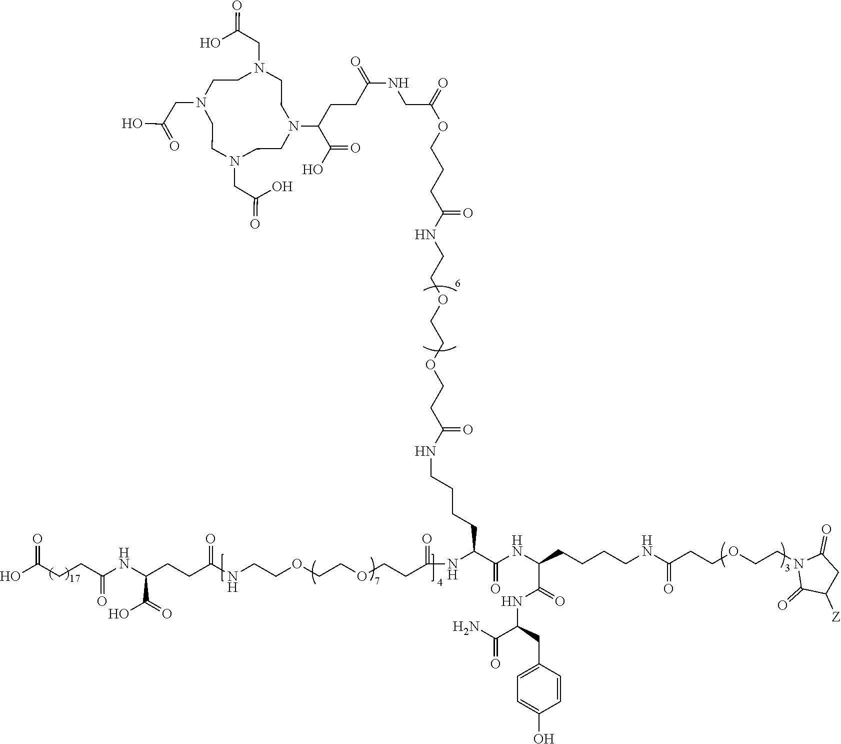 |
| C070 | HO—C20-gGlu-PEG8-PEG8-PEG8-PEG8-Lys(PEG8-ESL1-OEG-DOTAGA)-Lys(PEG3-Suc-Z)-Tyr-NH2 |
| | 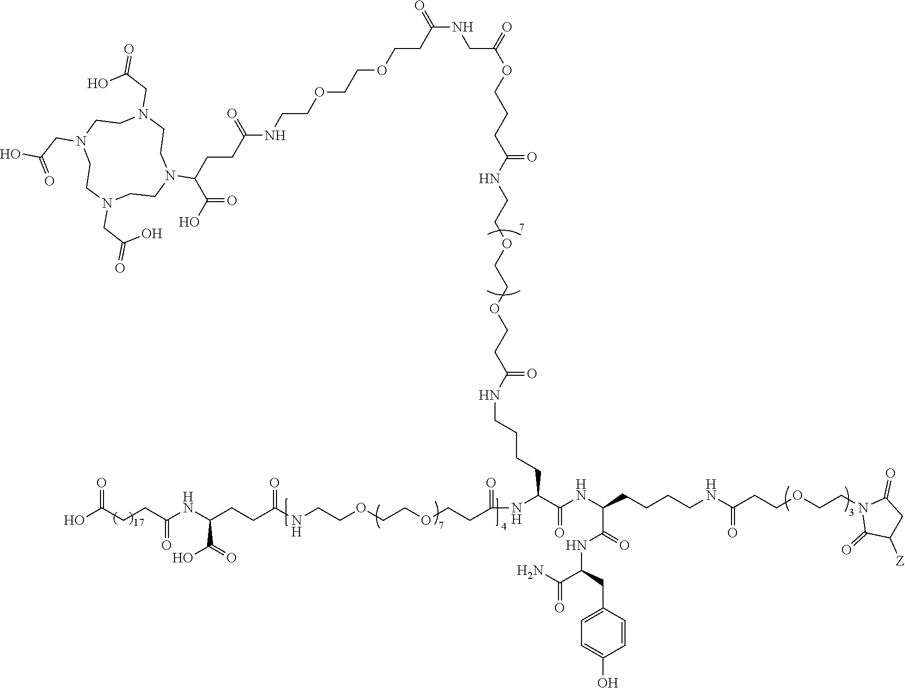 |
| C071 | HO—C20-gGlu-PEG8-PEG8-PEG8-PEG8-Lys(PEG6-ESL1-OEG-DOTAGA)-Lys(PEG3-Suc-Z)-Tyr-NH2 |
| | 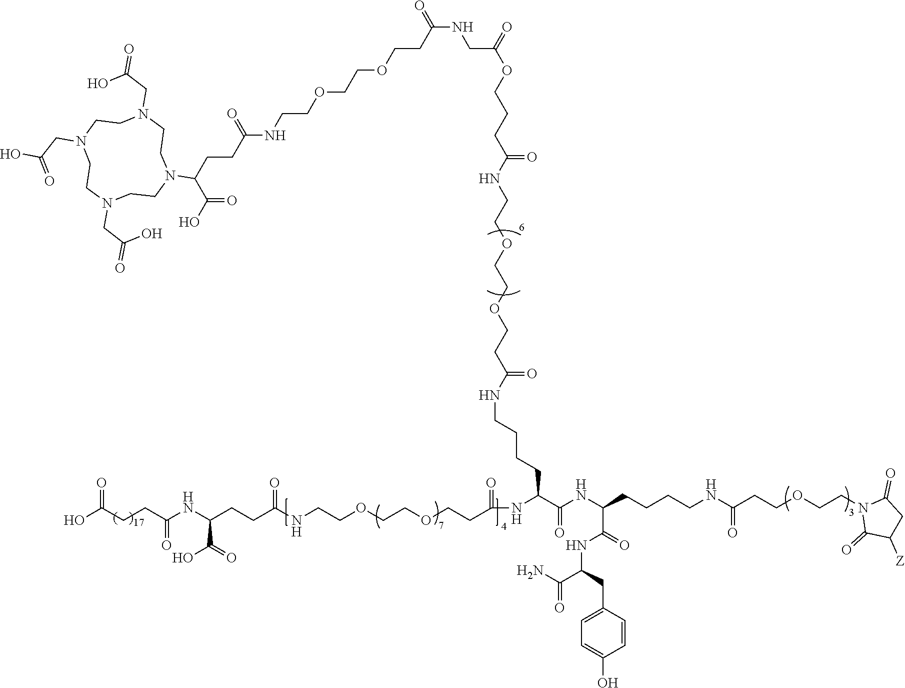 |
| C072 | HO—C20-gGlu-PEG8-PEG8-PEG8-PEG8-Lys(PEG8-ESL1-PEG4-DOTAGA)-Lys(PEG3-Suc-Z)-Tyr-NH2 |
| | 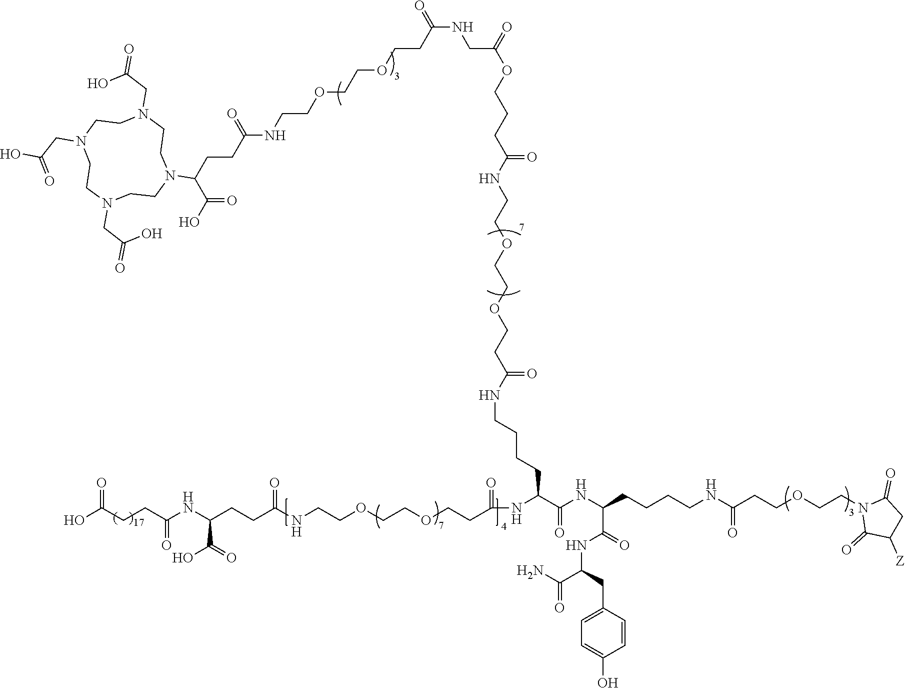 |
| C073 | HO—C20-gGlu-PEG8-PEG8-PEG8-PEG8-Lys(PEG8-ESL1-PEG5-DOTAGA)-Lys(PEG3-Suc-Z)-Tyr-NH2 |
| | 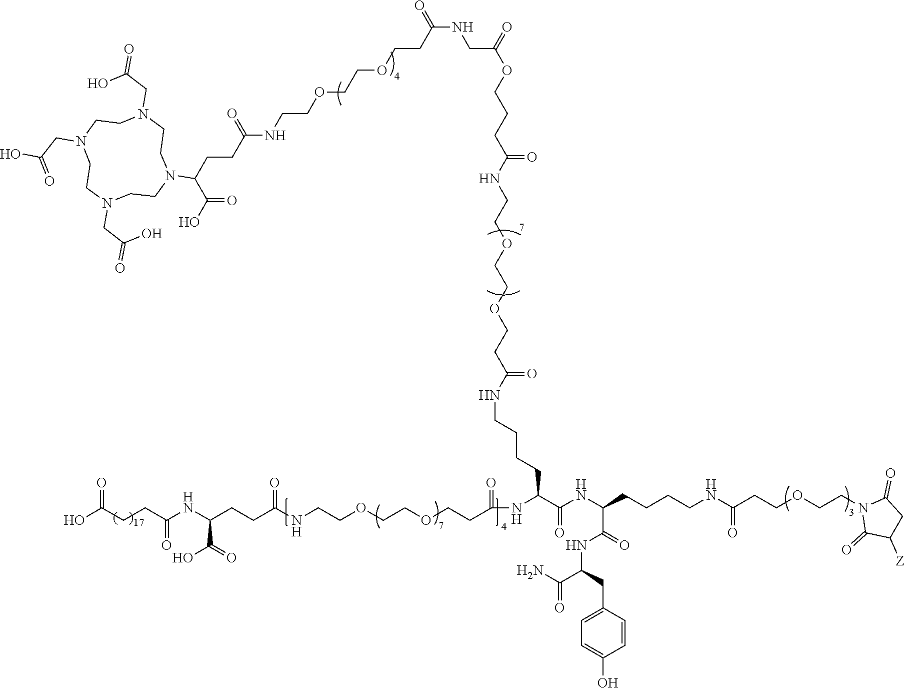 |
| C074 | HO—C20-gGlu-PEG8-PEG8-PEG8-PEG8-Lys(PEG8-ESL1-PEG6-DOTAGA)-Lys(PEG3-Suc-Z)-Tyr-NH2 |
| | 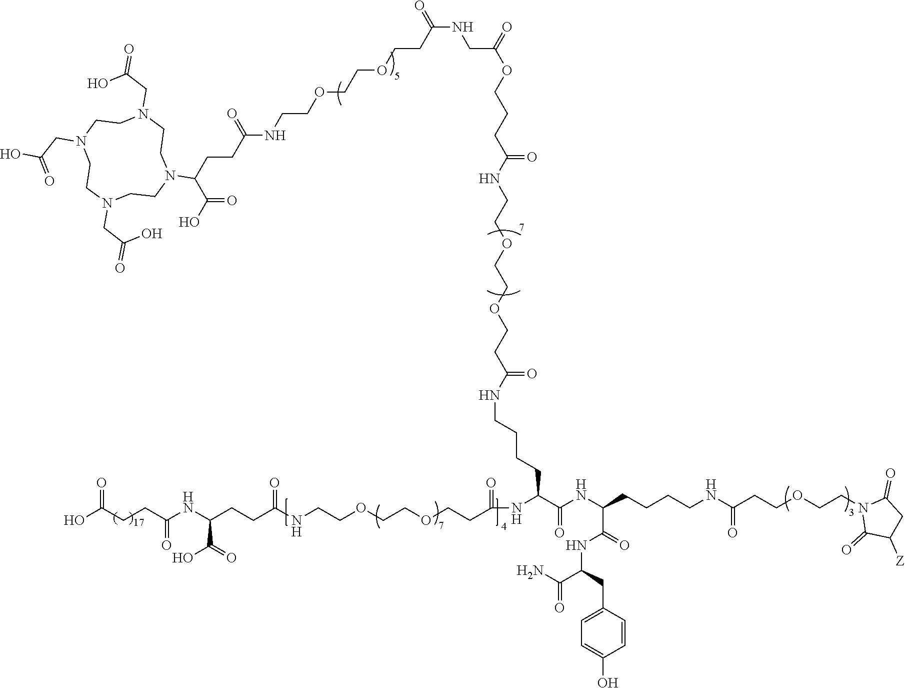 |
| C075 | HO—C20-gGlu-PEG8-PEG8-PEG8-PEG8-Lys(PEG8-ESL1-PEG7-DOTAGA)-Lys(PEG3-Suc-Z)-Tyr-NH2 |
| | 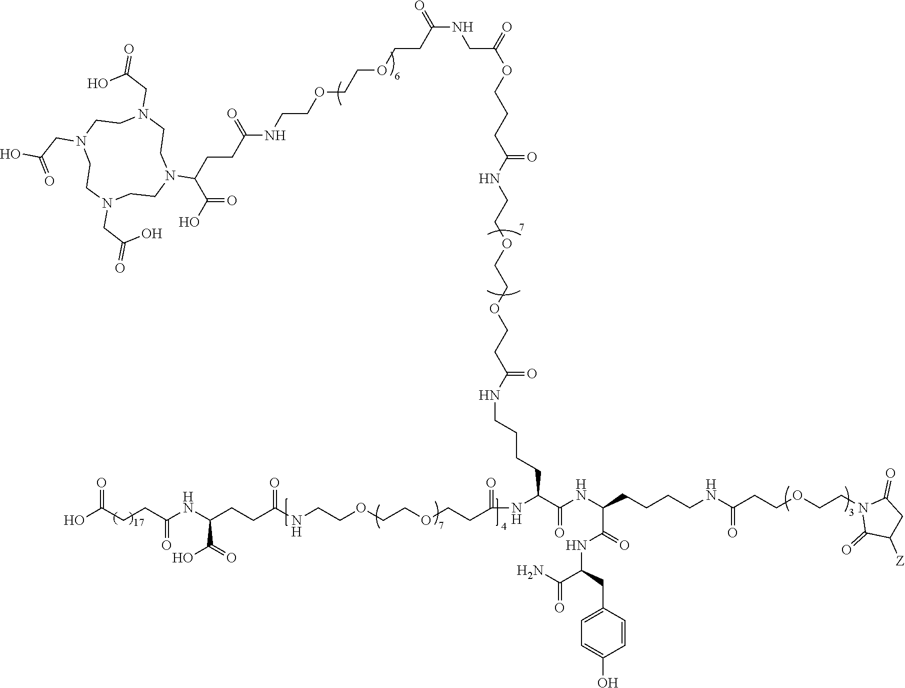 |
| C076 | HO—C20-gGlu-PEG8-PEG8-PEG8-PEG8-Lys(6hHA-Gly-PEG8-DOTAGA)-Lys(PEG3-Suc-Z)-Tyr-NH2 |
| | 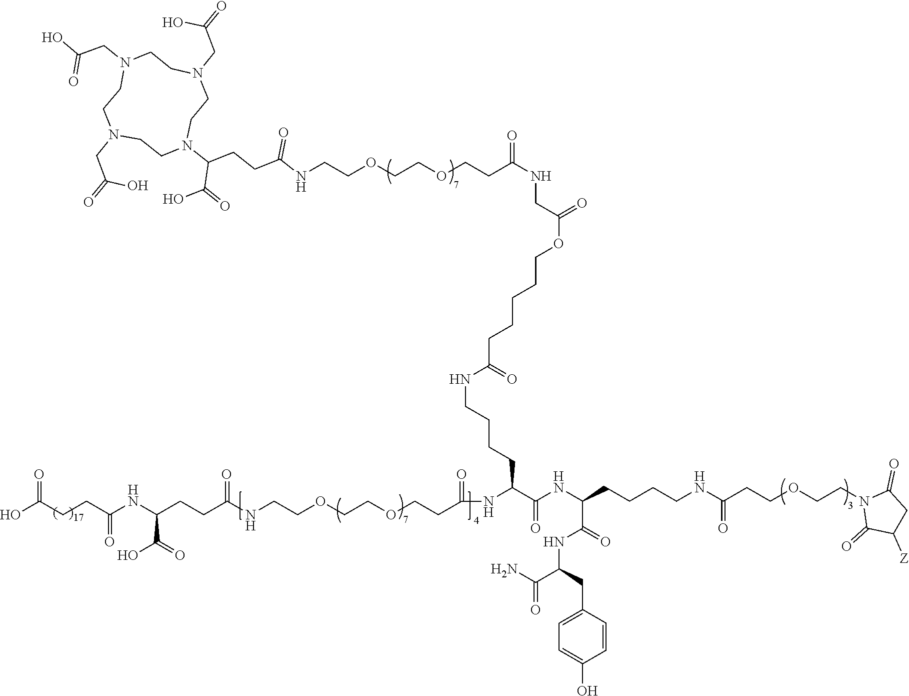 |
| C077 | HO—C20-gGlu-PEG8-PEG8-PEG8-PEG8-Lys(PEG8-6hHA-Gly-PEG6-DOTAGA)-Lys(PEG3-Suc-Z)-Tyr-NH2 |
| | 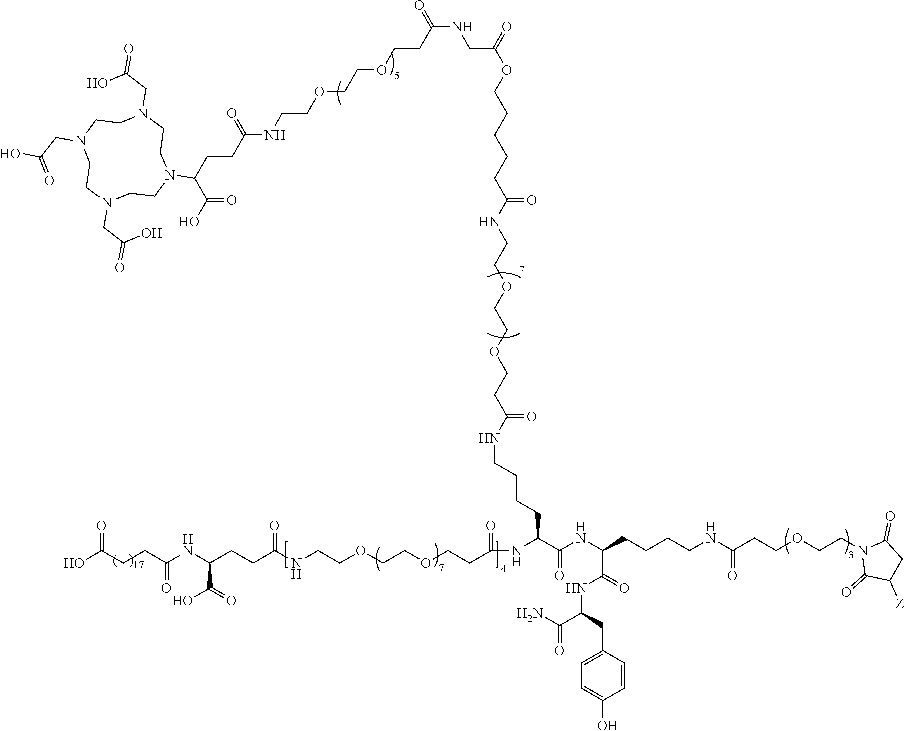 |
| C078 | HO—C20-gGlu-PEG8-PEG8-PEG8-PEG8-Lys(PEG8-OEG-PEG8-DOTAGA)-Lys(PEG3-Suc-Z)-Tyr-NH2 |
| | 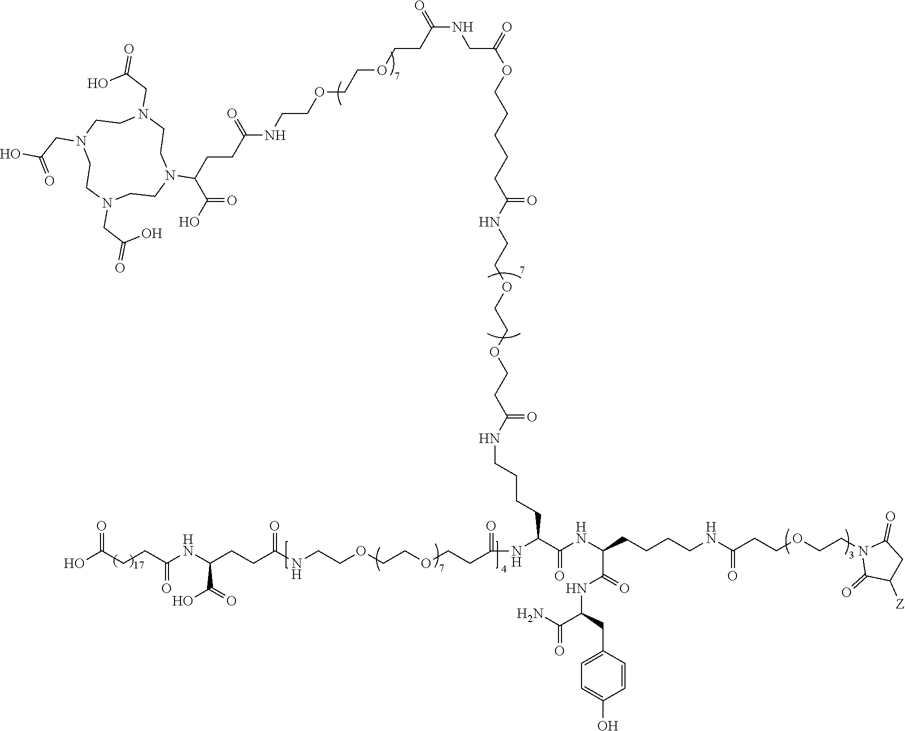 |
| C079 | HO—C20-gGlu-PEG8-PEG8-PEG8-PEG8-Lys(4hPA-Gly-DOTAGA)-Lys(PEG3-Suc-Z)-Tyr-NH2 |
| | 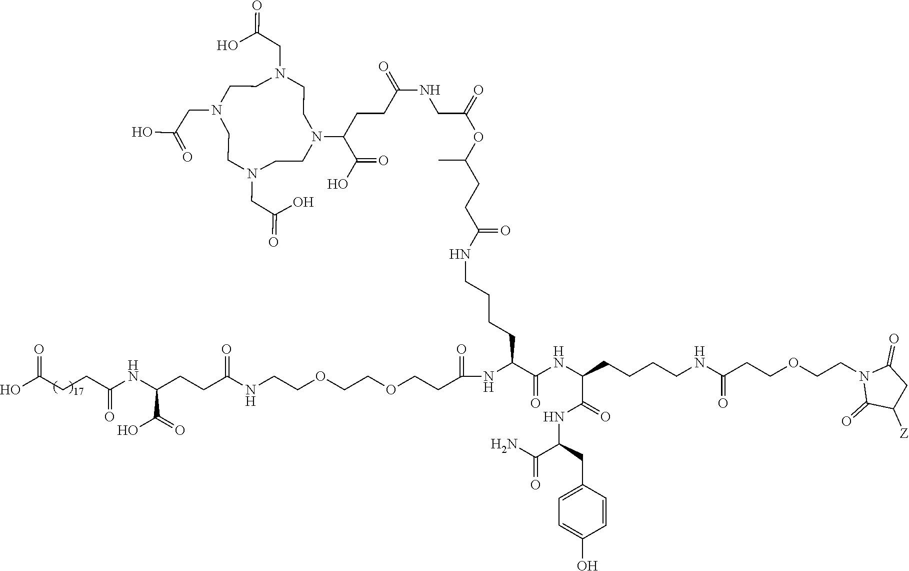 |
| C080 | HO—C20-gGlu-PEG8-PEG8-PEG8-PEG8-Lys(4hBA-Leu-DOTAGA)-Lys(PEG3-Suc-Z)-Tyr-NH2 |
| | 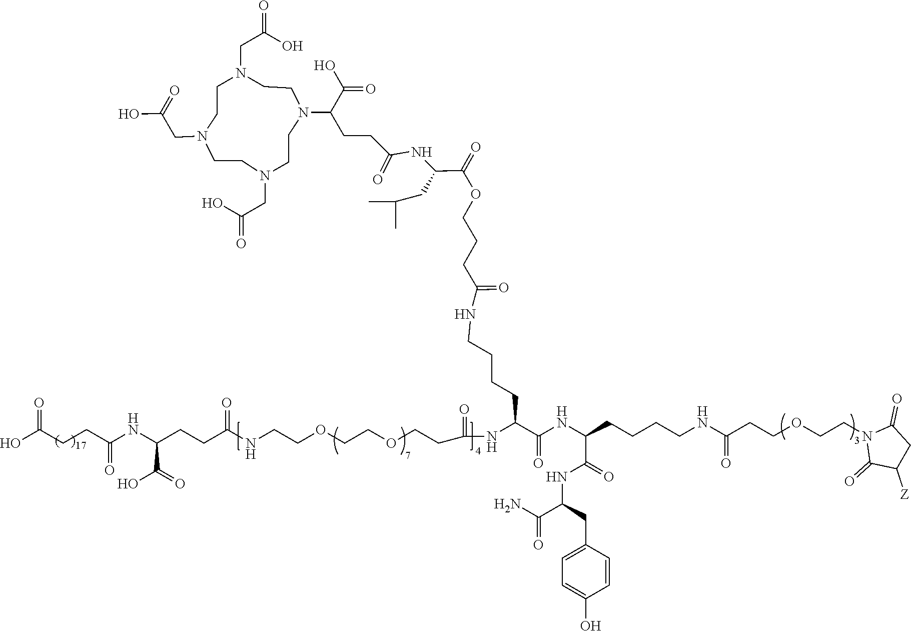 |
| C081 | HO—C20-gGlu-PEG8-PEG8-PEG8-PEG8-Lys(4hPA-Leu-DOTAGA)-Lys(PEG3-Suc-Z)-Tyr-NH2 |
| | 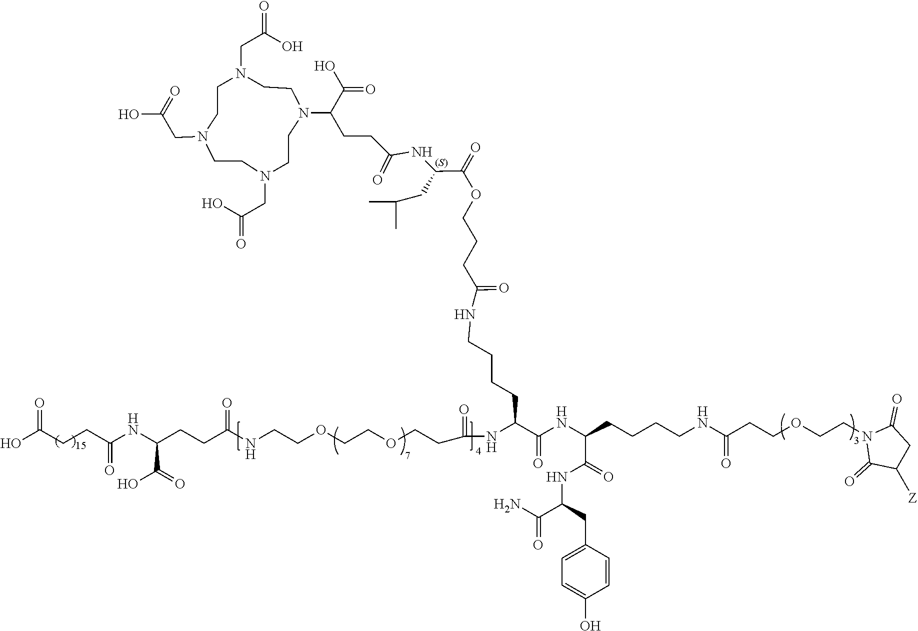 |
| C082 | HO—C20-gGlu-PEG8-PEG8-PEG8-PEG8-Gly-4hPA-Lys(DOTAGA)-Lys(PEG3-Suc-Z)-Tyr-NH2 |
| | 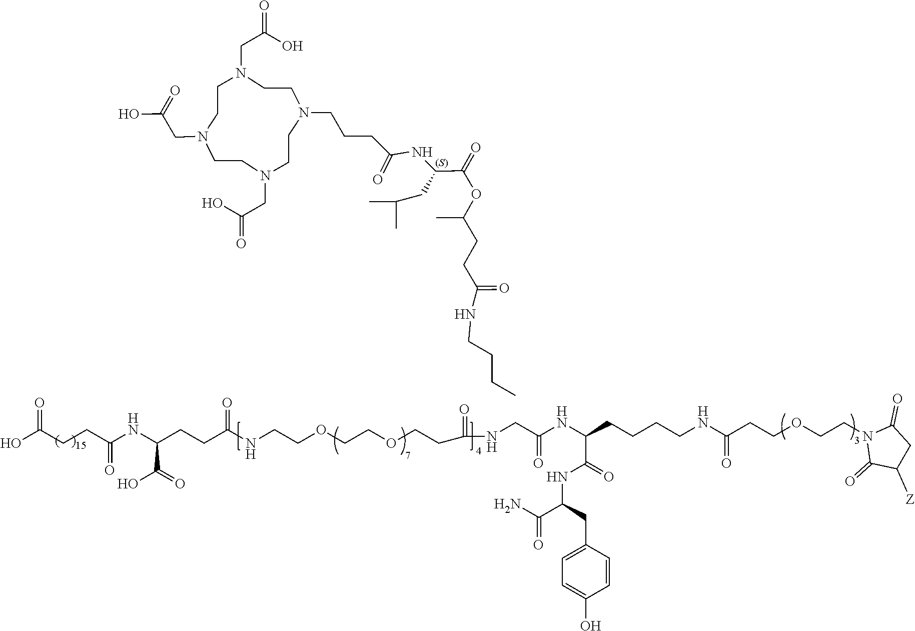 |
| C083 | HO—C20-gGlu-PEG8-PEG8-PEG8-PEG8-Leu-4hBA-Lys(DOTAGA)-Lys(PEG3-Suc-Z)-Tyr-NH2 |
| | 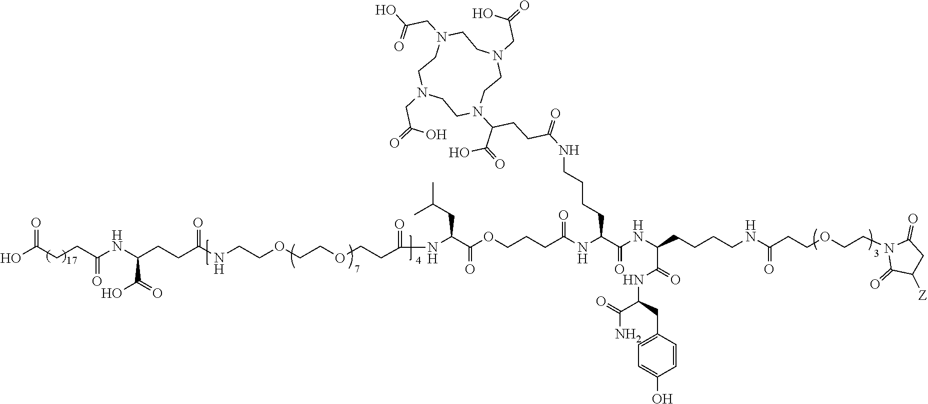 |
| C084 | HO—C20-gGlu-PEG8-PEG8-PEG8-PEG8-Leu-4hPA-Lys(DOTAGA)-Lys(PEG3-Suc-Z)-Tyr-NH2 |
| | 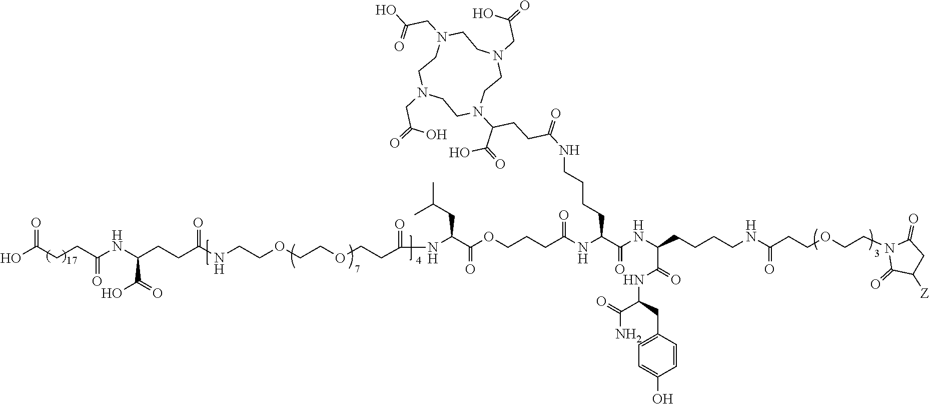 |
| C085 | HO—C20-gGlu-PEG8-PEG8-PEG8-PEG8-Gly-5hPA-Lys(DOTAGA)-Lys(PEG3-Suc-Z)-Tyr-NH2 |
| | 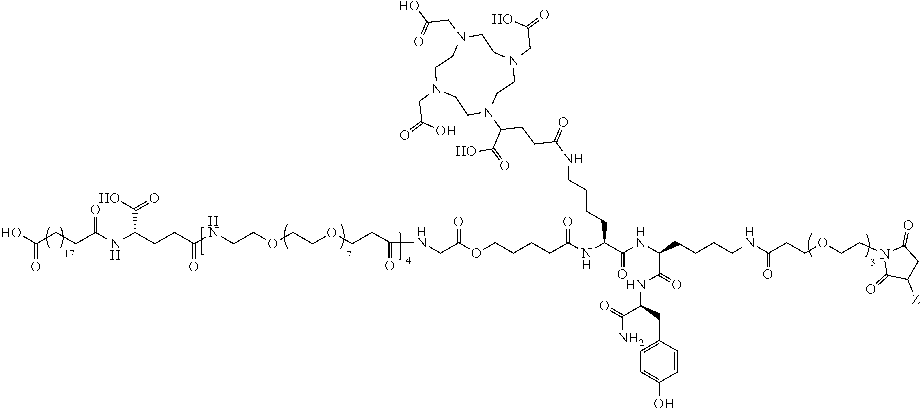 |
| C086 | HO—C20-gGlu-PEG8-PEG8-PEG8-PEG8-Gly-6hHA-Lys(DOTAGA)-Lys(PEG3-Suc-Z)-Tyr-NH2 |
| | 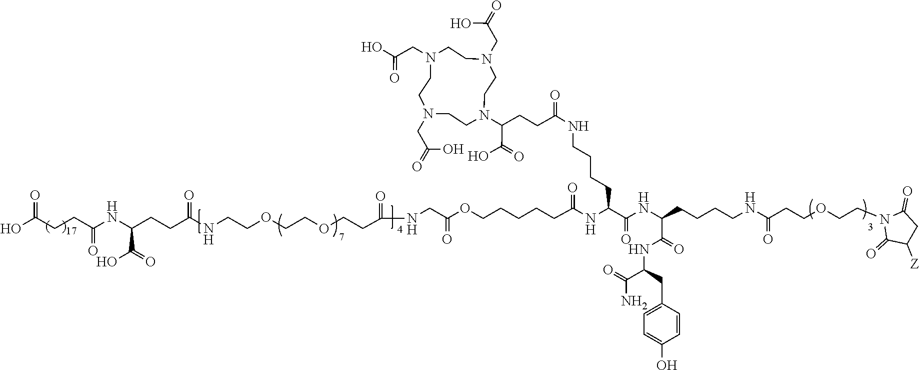 |
| C087 | HO—C20-gGlu-PEG8-PEG8-PEG8-PEG8-Lys(PEG8-4hPA-Gly-PEG8-DOTAGA)-Lys(PEG3-Suc-Z)-Tyr-NH2 |
| | 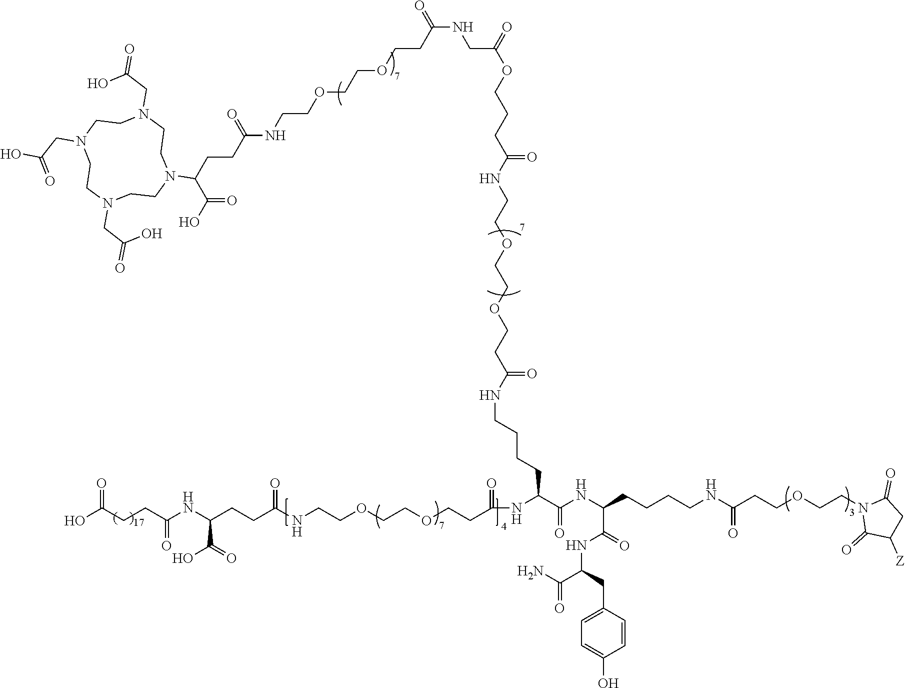 |
| C088 | HO—C20-gGlu-PEG8-PEG8-PEG8-PEG8-Lys(PEG8-4hPA-Ala-PEG8-DOTAGA)-Lys(PEG3-Suc-Z)-Tyr-NH2 |
| | 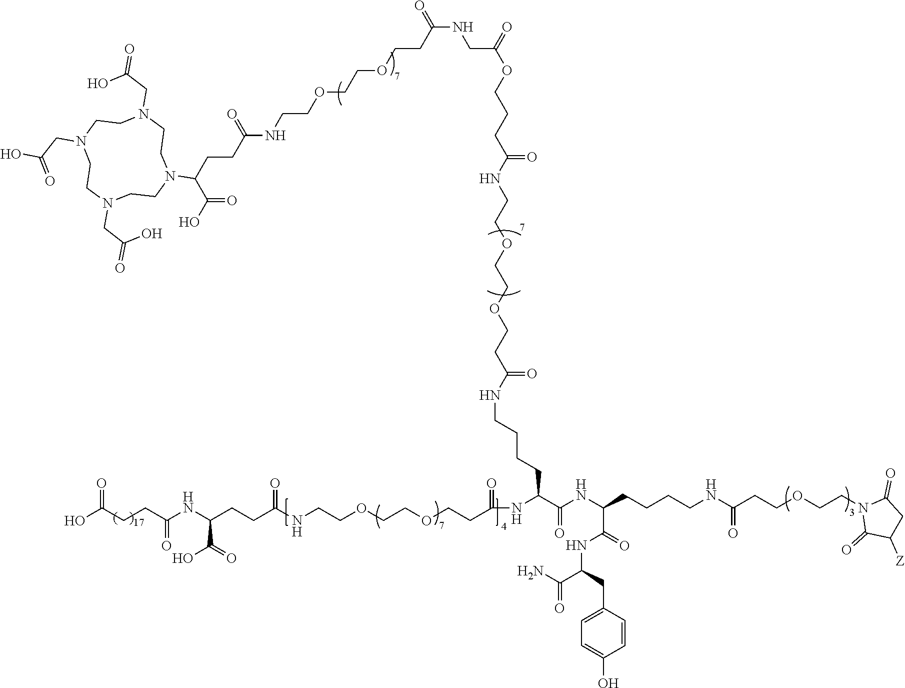 |
| C089 | HO—-gGlu-PEG8-PEG8-PEG8-PEG8-Lys(PEG8-4hBA-Ala-PEG8-DOTAGA)-Lys(PEG3-Suc-Z)-Tyr-NH2 |
| | 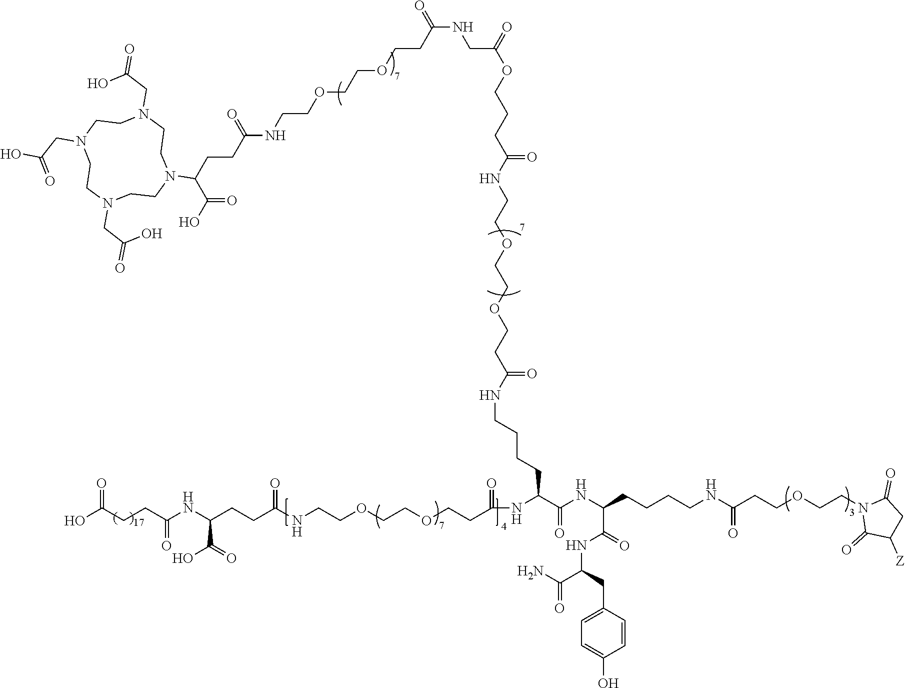 |
| C090 | HO—C20-gGlu-PEG8-PEG8-PEG8-PEG8-Lys(PEG8-4hBA-Leu-PEG8-DOTAGA)-Lys(PEG3-Suc-Z)-Tyr-NH2 |
| | 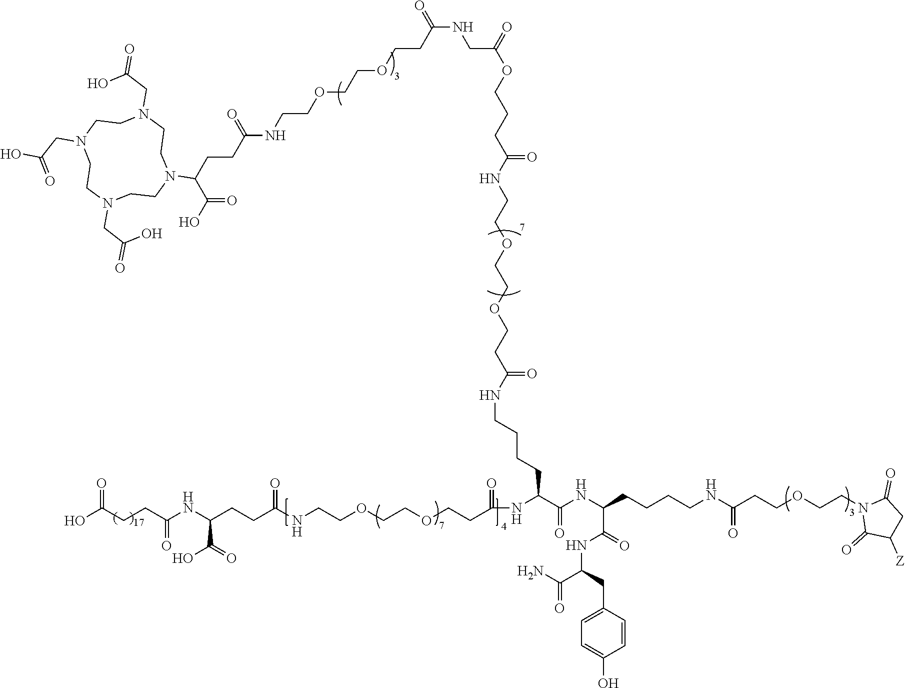 |
| C091 | HO—C20-gGlu-PEG8-PEG8-PEG8-PEG8-Lys(4hBA-Ala-DOTAGA)-Lys(PEG3-Suc-Z)-Tyr-NH2 |
| | 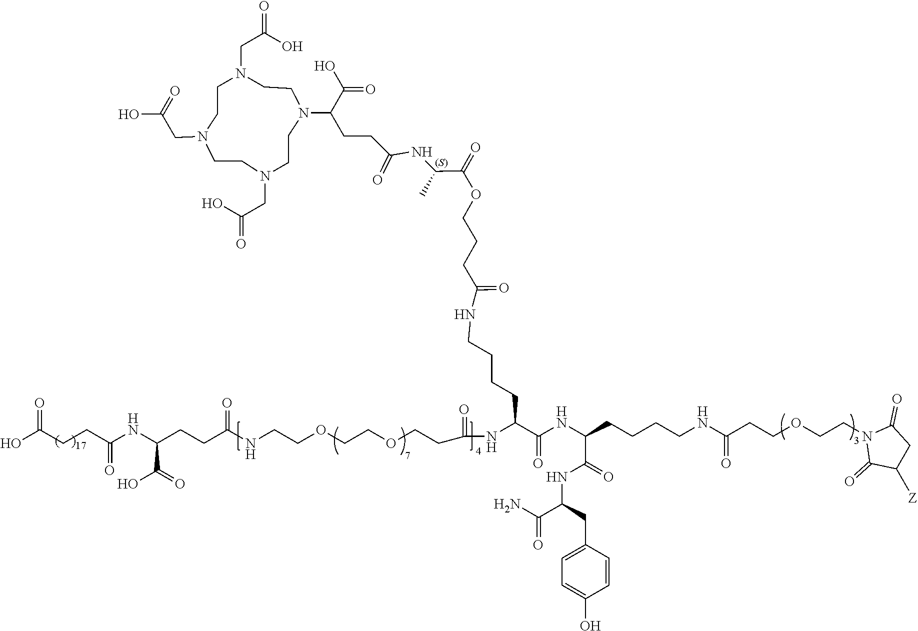 |
| C092 | [HO—C18-gGlu-PEG8-PEG8-PEG8-PEG8]2-Lys-Lys(PEG8-4hPA-Gly-PEG8-DOTAGA)-Lys(PEG3-Suc-Z)-Tyr-NH2 |
| | 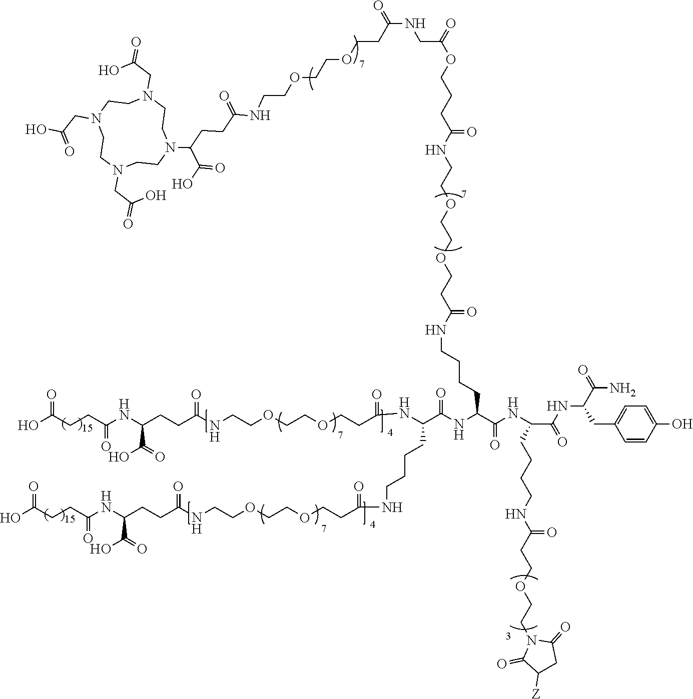 |
| C093 | [HO—C18-gGlu-PEG8-PEG8-PEG8-PEG8]2-Lys-Lys(PEG8-4hPA-Ala-PEG8-DOTAGA)-Lys(PEG3-Suc-Z)-Tyr-NH2 |
| | 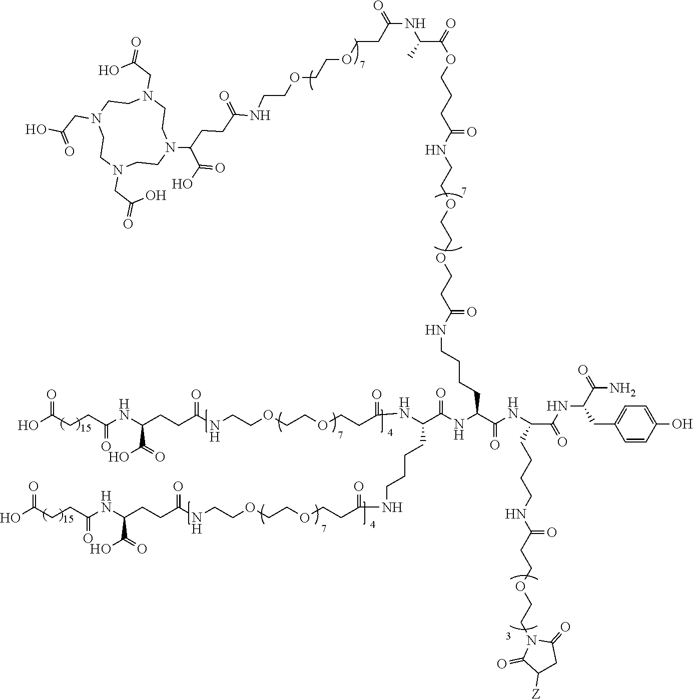 |
| C094 | HO—C20-gGlu-PEG8-PEG8-PEG8-PEG8-Lys(4hPA-Ala-DOTAGA)-Lys(PEG3-Suc-Z)-Tyr-NH2 |
| | 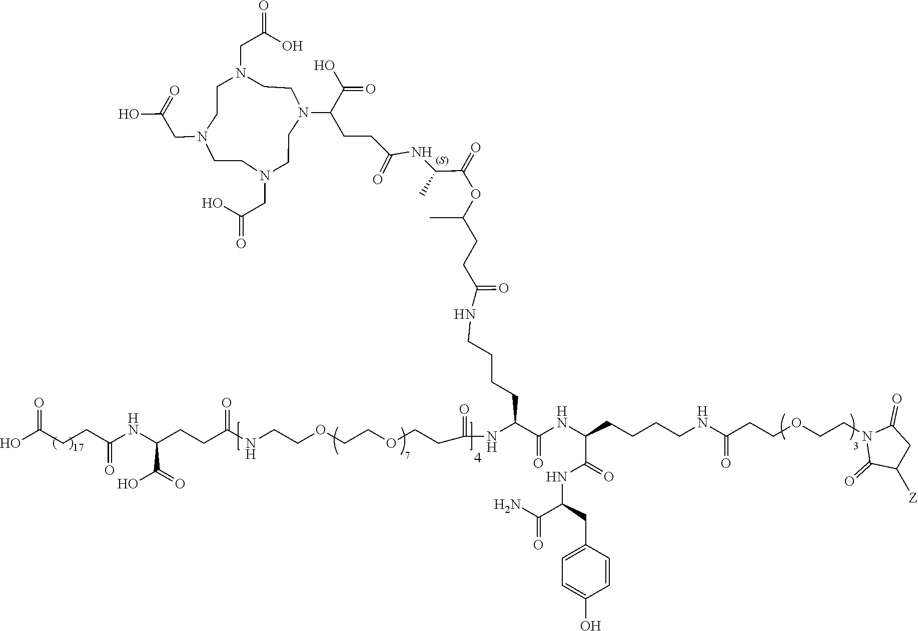 |
| C095 | [HO—C18-gGlu-PEG8-PEG8-PEG8-PEG8]2-Lys-Lys(PEG8-4hBA-Ala-PEG8-DOTAGA)-Lys(PEG3-Suc-Z)-Tyr-NH2 |
| | 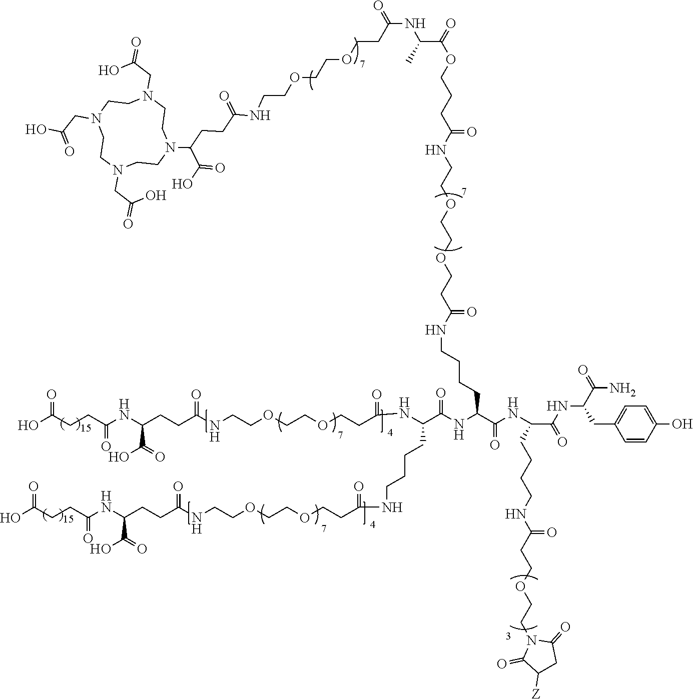 |
| C096 | [HO—C18-gGlu-PEG8-PEG8]2-Lys-Lys(PEG8-ESL1-PEG8-DOTAGA)-Lys(PEG3-Suc-Z)-Tyr-NH2 |
| | 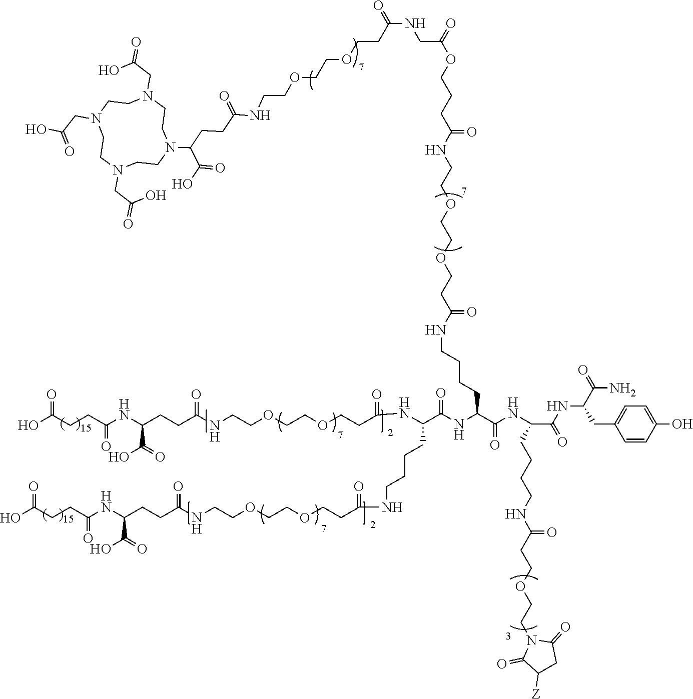 |
| C097 | HO—C18-gGlu-PEG8-PEG8-PEG8-PEG8-ESL1-Lys(DOTAGA)-Lys(PEG3-Suc-Z)-Tyr-NH2 |
| | 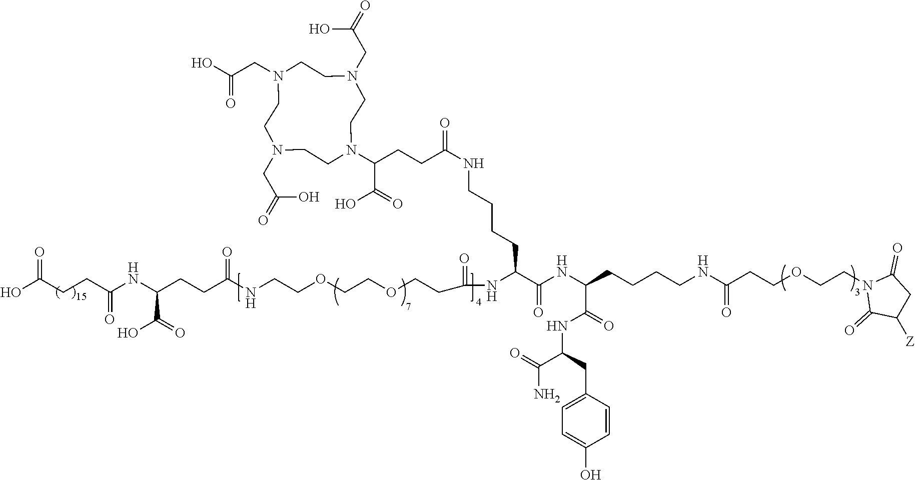 |
| C098 | HO—C18-gGlu-PEG8-PEG8-PEG8-PEG8]2-Lys-Lys(Gly-Gly-ESL1-Gly-Gly-DOTAGA)-Lys(PEG3-Suc-Z)-Tyr-NH2 |
| | 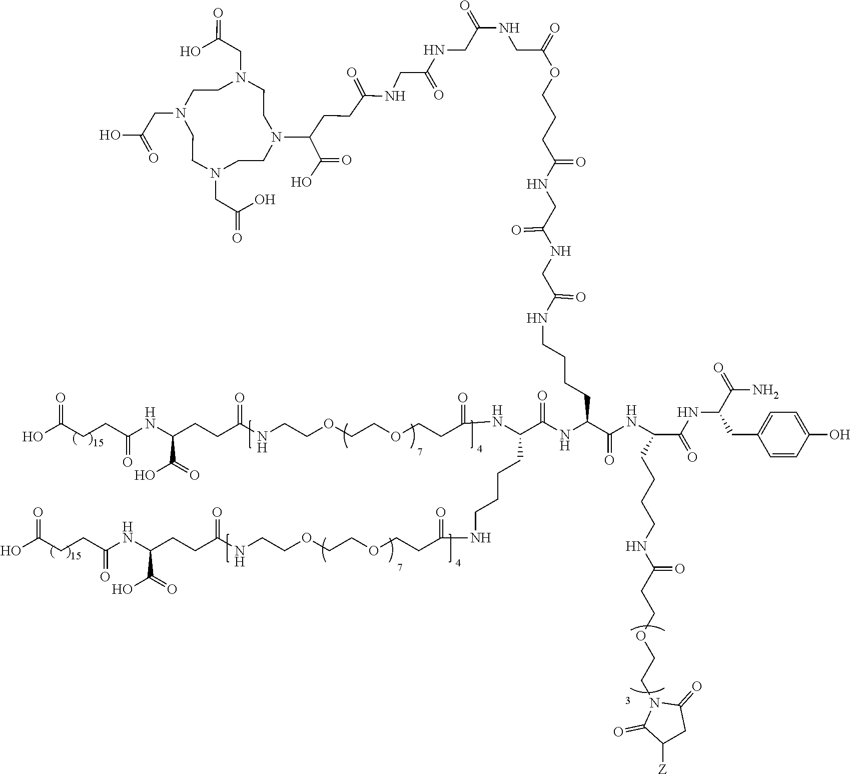 |
| C099 | [HO—C18-gGlu-PEG8-PEG8-PEG8-PEG8-]2-Lys-Lys(PEG8-ESL1-PEG8-DOTAGA)-Lys(PEG3-Suc-Z)-NH2 |
| | 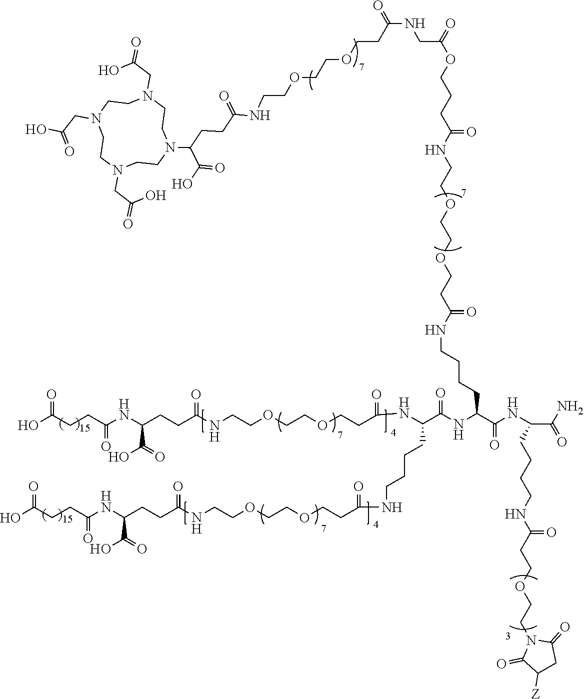 |
| C100 | HO—C20-gGlu-PEG8-PEG8-PEG8-PEG8-Lys(ESL1-DOTAGA)-Lys(PEG3-Suc-Z)-NH2 |
| | 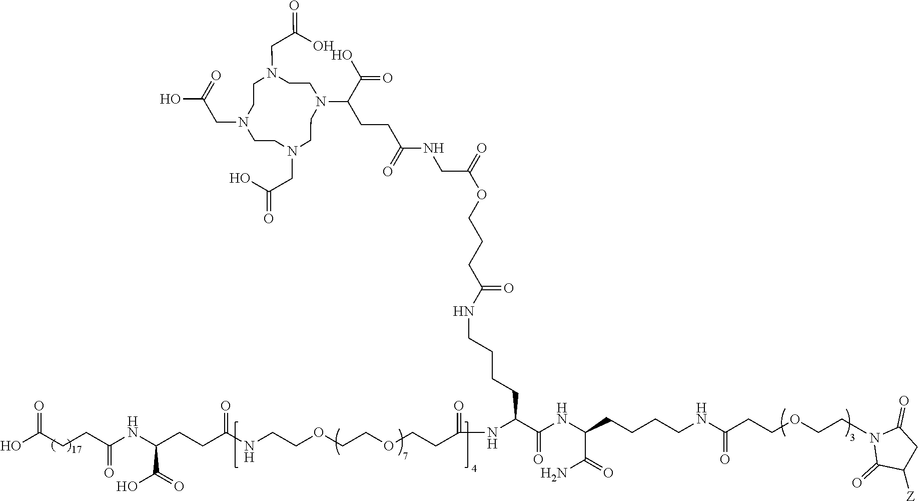 |
| C101 | HO—C18-gGlu-PEG8-PEG8-PEG8-PEG8-Lys(ESL1-DOTAGA)-Lys(PEG3-Suc-Z)-NH2 |
| | 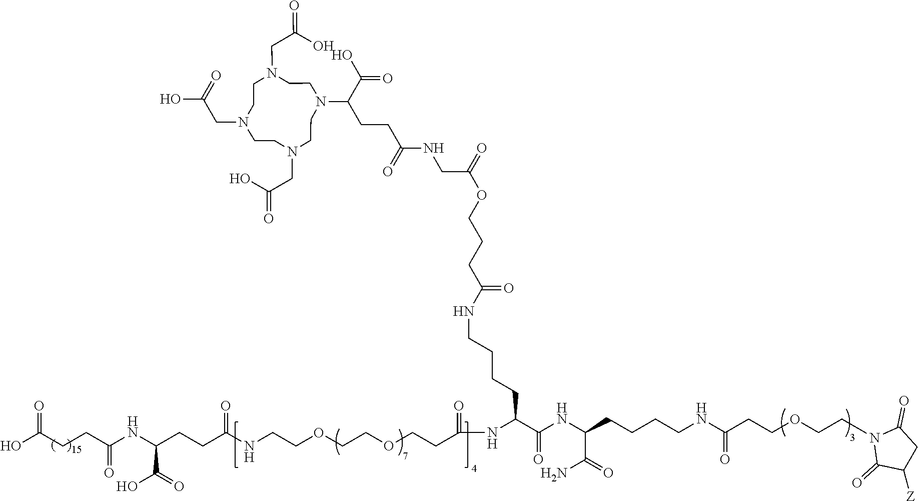 |

Any compound or structure given herein, is also intended to represent free form as well as salt form of the compound or structure. In some embodiments, the compound or structure is in neutral form or free form. In some embodiments, the compound or structure is a salt of the compound or structure as provided herein, such as a pharmaceutically acceptable salt. “Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid, and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide, and the like. Further examples of pharmaceutically acceptable salts include those listed in Berge et al., Pharmaceutical Salts, J. Pharm. Sci. 1977 Jan; 66(1):1-19. Pharmaceutically acceptable salts can be prepared in situ in the manufacturing process, or by separately reacting a purified compound of the disclosure in its free acid or base form with a suitable organic or inorganic base or acid, respectively, and isolating the salt thus formed during subsequent purification.

Also provided are stereoisomers, mixture of stereoisomers, and tautomers, of the compounds, structures, or conjugates described herein, such as but not limited to the conjugates of Formula I, I-A, I-B, I-C, I-D, I-E, I-F, I-G, I-H, I-I, I-J, I-K, I-L, I-A′, I-A′(a), I-A′(b), I-A′(c), I-A′(d), I-B′, I-C′ and I-D′.

### Compositions and Kits of the Application

The conjugates of the present application can be suitably formulated into compositions using one or more excipients. Accordingly, the present application also includes a composition comprising one or more conjugates of the application and an excipient. In some embodiments, the conjugates of the application are suitably formulated into pharmaceutical compositions for administration to subjects in a biologically compatible form suitable for administration in vivo. Accordingly, the present application further includes a pharmaceutical composition comprising one or more conjugates of the application and a pharmaceutically acceptable excipient. In embodiments of the application, the pharmaceutical compositions are used in the treatment of any of the diseases, disorders or conditions described herein.

The present application also includes a kit comprising one or more conjugates of Formula I as defined above, and optionally a package insert containing instructions for administration of the one or more conjugates of Formula I to a subject in need thereof.

The present application also includes a kit comprising one or more conjugates of Formula I as defined above, one or more radionuclides as defined above, and optionally a package insert containing instructions for administration of the one or more conjugates of Formula I and the one or more radioisotopes to a subject in need thereof.

In some embodiments, the kits are for use in imaging. In some embodiments, the kits are for use in therapy. In some embodiments, the kits are for use in treating a disorder such as cancer. In some embodiments, the kits are adapted and/or arranged to carry out any method of the present application.

### Methods and Uses of the Application

Accordingly, the present application also includes a method of treating a disease or disorder comprising administering a therapeutically effective amount of the conjugate or pharmaceutical composition of the present application to a subject in need thereof. The present application also includes a use of the conjugate or pharmaceutical composition of the present application for treatment of a disease or disorder as well as a use of the conjugate or pharmaceutical composition of the present application for the preparation of a medicament for treatment of a disease or disorder. The application further includes the conjugate or pharmaceutical composition of the present application for use in treating a disease or disorder, such as cancer.

The application also includes a method of inhibiting proliferative activity in a cell, comprising administering an effective amount of the conjugate or pharmaceutical composition of the present application to the cell. The present application also includes a use of the conjugate or pharmaceutical composition of the present application for inhibition of proliferative activity in a cell as well as a use of the conjugate or pharmaceutical composition of the present application for the preparation of a medicament for inhibition of proliferative activity in a cell. The application further includes the conjugate or pharmaceutical composition of the present application for use in inhibiting proliferative activity in a cell.

The present application also includes a method of imaging a tissue in a subject by administering an imaging effective amount of the conjugate or pharmaceutical composition of the present application for use in imaging to a subject in need thereof.

The present application also includes a use of the conjugate or pharmaceutical composition of the present application for use in imaging for imaging a tissue well as a use of the conjugate or pharmaceutical composition of the present application for use in imaging for the preparation of a medicament for imaging a tissue. The application further includes the conjugate or pharmaceutical composition of the present application for use in imaging a tissue.

The present application also includes a method of diagnosing cancer in subject by administering a diagnostic effective amount of the conjugate or pharmaceutical composition of the present application. The present application also includes a use of the conjugate or pharmaceutical composition of the present application for diagnosing cancer in well as a use of the conjugate or pharmaceutical composition of the present application for diagnosing cancer. The application further includes the conjugate or pharmaceutical composition of the present application for use in imaging for use in diagnosing cancer.

In some embodiments, the treating disease or disorder such as cancer, or imaging a tissue or diagnosing cancer having a greater uptake of the conjugate or pharmaceutical composition of the present application in a target cells, tissues and/or organs compared to off-target cells, tissues and/or organs. In some embodiments, the treating disease or disorder such as cancer, or imaging a tissue or diagnosing cancer having a greater uptake of the conjugate or pharmaceutical composition of the present application in a target cells, tissues and/or organs compared to off-target cells, tissues and/or organs, and compared to otherwise identical conjugates or complexes except for the presence of the cleavable moiety(ies) in the conjugates or complexes of the application.

To minimize the amount of radionuclide exposure to the normal organs and to purposely degrade the radioligand to metabolites that are less likely accumulate in non-target receptor expressing cells, the present inventors have introduced a cleavable linker to a radioligand conjugate.

In an embodiment, the subject is a mammal. In another embodiment, the subject is human. In an embodiment, the subject is a non-human animal. In an embodiment, the subject is canine. In an embodiment, the subject is feline. Accordingly, the conjugates, methods and uses of the present application are directed to both human and veterinary diseases, disorders and conditions.

### Methods of Preparing the Conjugates

In another aspect, the present application relates to a method of preparing the conjugate of the present application, comprising conjugating the compound with the tumor targeting moiety as described herein.

In some embodiments, the method of preparing the conjugate comprises: (i) introducing into a host cell a nucleic acid encoding Z; (ii) culturing the host cell under conditions that allow expression of Z; (iii) isolating Z; and (iv) conjugating Z with the compound of formula (I-X) via L^{Z(i)} of the compound:

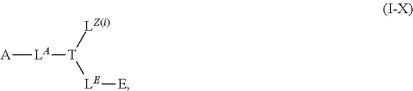

wherein: (1) L^{Z(i)} is a linker comprising a thiol-reactive group or an amine-reactive group; (2) T is a branching group which is at least trivalent; (3) E is an effector moiety, wherein E is conjugated to T via a linker L^{E}, and (4) A is a potency enhancing moiety, wherein A is conjugated to T via a linker L^{A}.

In some embodiments, the host cell used in the preparation method of the present application is a eukaryotic cell or a prokaryotic cell. In some embodiment, the prokaryotic cell is E. Coli. In some embodiments, the eukaryotic cell is a yeast cell or a mammalian cell.

In some embodiments, the eukaryotic cell is a mammalian cell. In some embodiments, the eukaryotic cell includes but is not limited to HEK293 cell, CHO cell, and BHK cell.

In some embodiments of the method of present application, the compound is conjugated with Z under a molar ratio (compound:Z) from about 10:1 to about 1:1. In some embodiments, the compound is conjugated with Z under a molar ratio (compound:Z) from about 10:1 to about 5:1. In some embodiments, the compound is conjugated with Z under a molar ratio (compound:Z) from about 5:1 to about 1:1. In some embodiments, the compound is conjugated with Z under a molar ratio (compound:Z) from about 5:1 to about 3:1. In some embodiments, the compound is conjugated with Z under a molar ratio (compound:Z) from about 3:1 to about 1:1. In some embodiments of the method of present application, the compound is conjugated with Z under a molar ratio (compound:Z) of about 10:1, 9:1, 8:1, 7:1, 6:1, 5:1, 4:1, 3:1, 2:1 or 1:1. In some embodiments, the compound is conjugated with Z under a molar ratio (compound:Z) of about 3:1. In some embodiments, the compound is conjugated with Z under a molar ratio (compound:Z) of about 1:1.

Conjugates of the present application can be prepared by various synthetic processes.

The choice of particular structural features and/or substituents may influence the selection of one process over another. The selection of a particular process to prepare a given conjugate of the present application or comparator conjugates is within the purview of the person of skill in the art. Some starting materials for preparing conjugates of the present application are available from commercial chemical sources. Other starting materials, for example as described below, are readily prepared from available precursors using straightforward transformations that are well known in the art.

In some embodiments, part of the conjugate of Formula, such as the compound, can be prepared using solid phase peptide synthesis (SPPS) or solution phase coupling techniques known in the art, for example, using the synthetic procedures found in Stewart and Young, 1984, Solid Phase Synthesis, Second Edition, Pierce Chemical Co., Rockford, Ill.; Fields and Noble, 1990, “Solid phase peptide synthesis utilizing 9-fluorenylmethyloxycarbonyl amino acids,” Int. J. Pept. Protein Res. 35:161-214; Geysen et al., 1987, J. Immunol. Methods 102:259-274.

Accordingly, in some embodiments, in SPPS, an Nα-protected linker group, such as a tert-butoxycarbonyl (Boc) or 9-fluorenylmethyloxycarbonyl (Fmoc) amino acid linker group, is activated at the α-carbonyl and coupled with the deprotected Na functionality of the solid phase support. The newly added Nα-protected linker group is then deprotected and coupled to the next Nα-protected linker group if necessary until the final cleavage step. It would be appreciated by the person skilled in the art the chemistry of the coupling, deprotection, and final cleavage step of the linker from the solid phase support depends on choice of a N-protecting group. In some embodiments, the cleavage is accomplished by treatment with acid, for example trifluoro acetic acid (TFA) optionally in the presence of scavenger reagents such as triisopropylsilane. In some embodiments, when the α N-protecting group is Fmoc, cleavage in acid will also result in deprotection of the side chains.

Therefore, in an exemplary embodiment, part of the conjugate of Formula I or comparator conjugate, such as the linker compound, can be prepared by using fluorenylmethyloxycarbonyl (Fmoc) solid-phase peptide synthesis chemistry known in the art. Accordingly, in some embodiments, the conjugates of Formula I or fragments therefore are prepared, manually or by using automated multiple solid-phase peptide synthesizer, using a Wang resin, Rink Amide-MBHA or equivalent resin and Fmoc-protected linker group derivatives with suitable side-chain protections such as Fmoc-Ala-OH, Fmoc-Arg(Pbf)-OH, Fmoc-Asn(Trt)-OH, Fmoc-Asp(OtBu)-OH, Fmoc-Cys(Trt)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Met-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, Fmoc-Val-OH, Fmoc-Lys(Mtt)-OH, Fmoc-Aib-OH, Fmoc-Nle-OH, Fmoc-8-amino-3,6-dioxaoctanoic acid (Fmoc-OEG-OH), Fmoc-Glu-OtBu, octadecanedioic acid mono-tert-butyl ester, nonadecanedioic acid mono-tert-butyl ester, eicosanedioic acid mono-tert-butyl ester and tetradecanedioic acid mono-tert-butyl ester. The resin is swelled using a suitable solvent such as combination of dichloromethane (DCM) and (DMF). Prior to each coupling step the base-labile Nα- protecting group Fmoc is cleaved off from the Fmoc protected linker groups using a suitable base such as piperidine in a suitable solvent such as DMF for time to cleave to cleave the Fmoc protecting group, for example, about 10-15 min. The resin is then subsequently washed with a suitable solvent such as the DMF to, for example, remove piperidine. An excess amount of the Fmoc-linker group (e.g., 4 to 8 molar equivalent) is then coupled using coupling agents known in the art, for example, N,N′-diisopropylcarbodiimide (DIC) and ethyl cyanohydroxyiminoacetate (Oxyma, e.g Oxyma Pure®) or (Benzotriazol-1-yloxy)tripyrrolidinophosphonium hexafluorophosphate (PyBOP) and (1-Hydroxybenzotriazole (HOBt), in a suitable solvent such as DMF for about 1 to about 2 hours and then further washed with a suitable solvent, such as DMF. The coupling step is repeated once for each linker group.

When necessary, the methyltrityl (Mtt) group of the Fmoc-Lys(Mtt)-OH (i.e., N-α-Fmoc-N-ε-4-methyltrityl-L-lysine) linker group or the deprotected Lys(Mtt)-residue in the linker fragment is removed by treating the group or residue with hexafluoroisopropanol (HFIP) in a suitable solvent such as dichloromethane (DCM) (e.g. about 30% v/v) for suitable amount of time, for example, about 1 hour, followed by washing the resin with the suitable solvent and repeating the treatment with HFIP in DCM with a final washing with DCM after treatment.

After coupling, the linker compound or fragment thereof can be cleaved from the solid phase by treatment with a suitable acid for example, trifluoroacetic acid (TFA) and optionally in the presence of a trialkylsilane such as triisopropylsilane (TIP) and water and then precipitated with a suitable solvent such as diethyl ether. The product is dissolved in a suitable solvent such as water and acetonitrile and purified using high-performance liquid chromatography (HPLC) such as reversed phase HPLC using a suitable solvent or solvent mixture such as water with acetonitrile and TFA with an increasing gradient of acetonitrile. Relevant fractions are checked by analytical UPLC. Fractions containing the pure target conjugates are pooled and freeze-dried.

The chelating group such as DOTA is conjugated to the linker fragment, for example, F-amine of a lysine residue of the linker fragment or the linker fragment attached to the tumour binding group and/or potency enhancing group using active ester chemistry known in the art. For example, DOTA is combined with the linker fragment in the presence of a base such as an amine.

The chelating groups can be synthesized through methods known in the art or are commercially available. For example, DOTA is available from Sigma-Aldrich (St. Louis, Missouri, United States).

In some embodiments, when the linker compound comprises a triazole (i.e., X³ is triazole), the triazole ring is incorporated in the linker group by reacting a suitable azide precursor conjugate with a suitable acetylene precursor conjugate using click reaction conditions (e.g., Tetrahedron 2016, 72, 5257-5283; Tetrahedron 2016, 72, 6136-6141).

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art.

Examples of transformations are given herein, and it is to be understood that the described transformations are not limited only to the generic groups or substituents for which the transformations are exemplified. References and descriptions of other suitable transformations are given in “Comprehensive Organic Transformations —A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

The following non-limiting examples are illustrative of the present application.

### EXAMPLES

### Example 1. Synthesis of Exemplary Compounds of Formula (I-X)

### 1.1 Reagents

The Fmoc-protected amino acid derivatives used, unless specifically stated otherwise, were the standard recommended: Fmoc-Ala-OH, Fmoc-Arg(Pbf)-OH, Fmoc-Asn(Trt)-OH, Fmoc-Asp(OtBu)-OH, Fmoc-Cys(Trt)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Leu-OH, Fmoc-Met-OH, Fmoc-Phe-OH, Fmoc-D-Phe-OH, Fmoc-Pro-OH, Fmoc-D-Pro-OH, Fmoc-Ser(tBu)-OH, Fmoc-Thr(tBu)-OH, Fmoc-Trp(Boc)-OH, Fmoc-D-Trp(Boc)-OH, Fmoc-Tyr(tBu)-OH, Fmoc-Val-OH, Fmoc-Lys(Mtt)-OH, Fmoc-Lys(ivDde)-OH, Fmoc-Glu-OtBu, Fmoc-8-amino-3,6-dioxaoctanoic acid (Fmoc-OEG-OH), Fmoc-8-amino-octanoic acid (Fmoc-Aoc-OH), Fmoc-PEG₁-OH (Fmoc-HN-CH₂CH₂O—CH₂CH₂COOH), Fmoc-PEG₃-OH (Fmoc-HN-[CH₂CH₂O]₃—CH₂CH₂COOH), Fmoc-PEG₆-OH (Fmoc-HN-[CH₂CH₂O]₆—CH₂CH₂COOH) etc. Other reagents used included DOTA-tris(tert-butyl ester), DOTAGA-tetra (t-Bu ester), etc.

### 1.2 C-Terminal Acid Peptides

The C-terminal acid peptides were prepared using Wang resin. The coupling of the first C-terminal residue started with swelling Wang resin (loading 1.1 mmol/g, 5.0 mmol) in DCM (50 mL) in a SPPS reaction vessel with N₂ bubbling for 30 min. The resin was then drained and washed with DMF (50 mL) 3 times. In a separate flask, a mixture of Fmoc-Thr(tBu)-OH (15 mmol), DIC (15 mmol) and 4-dimethylaminopyridine (DMAP) (0.5 mmol) in DMF (60 mL) was stirred for 15 min at room temperature before being transferred to the above reaction vessel. The resulting mixture was bubbled with N₂ for 4 h, then drained and washed with DMF (50 mL) 6 times, followed by the addition of DMF (60 mL), acetic anhydride (50 mmol) and DMAP (0.50 mmol). The resulting mixture was bubbled with N₂ for 2 h, then drained and the resin was washed with DMF (50 mL) 6 times.

### 1.3 C-Terminal Amide Peptides

The C-terminal amide peptides were prepared using Rink Amide-MBHA resin. The first C-terminal residue (Fmoc-Thr(tBu)-OH) was attached following a standard amide bond coupling conditions using PyBOP/HOBt/DIEA as coupling reagents. The resin was washed with DMF 6 times before the coupling of the second residue.

### 1.4 Standard Solid-Phase Assembly Protocol

The synthesis was performed using Fmoc-based chemistry manually. The stepwise assembly was conducted following the below steps:
1) Pre-swelling the resin with DCM and DMF;
2) Removing the Fmoc group by 20% piperidine; 2 treatments, 10 min each;
3) Washing the resin with DMF to remove piperidine;
4) Adding Fmoc-amino acid (1 mmol), PyBOP (1 mmol), HOBt (0.2 mmol) and DMF (5 mL) to a reaction vessel containing 0.20 mmol resin followed by addition of DIEA (2 mmol), the resulting mixture was mixed by bubbling nitrogen for 1-2 h;
5) Draining the resin, and washed the resin with DMF 6 times
6) When needed, the N-epsilon-lysine Mtt protective group could be removed by treating the resin with 30% (v/v) HFIP in DCM for 1 h twice; drained the resin and washed it with DCM and DMF;
7) The final wash before resin cleavage was done with DMF (3 times), DCM (3 times) and MeOH (3 times), respectively.

### 1.5 Resin Cleavage and Peptide Purification

After solid-phase peptide assembly was completed, the resin was subject to a 1.5-3 h treatment of TFA/triisopropylsilane (TIS)/H₂O (95:2.5:2.5, v/v/v). The resin was filtered off and washed one time with TFA, the combined filtrate was treated with methyl tert-butyl ether (MTBE) to precipitate the crude peptide out of the solution. The precipitate was collected by centrifugation and washed 3 times with diethyl ether, briefly dried, then subjected to purification by using a reverse phase preparative HPLC system (Waters Delta Prep 4000) with a C18-reverse phase column. Mobile phases: A: 0.1% TFA/H₂O; B: 0.1% TFA/ACN. Relevant fractions were analyzed by analytical ultra-performance liquid chromatography (UPLC). The pure fractions were pooled and freeze-dried affording the product as a white lyophilized powder.

### 1.6 Synthesis of Exemplary Functional Group ESL1

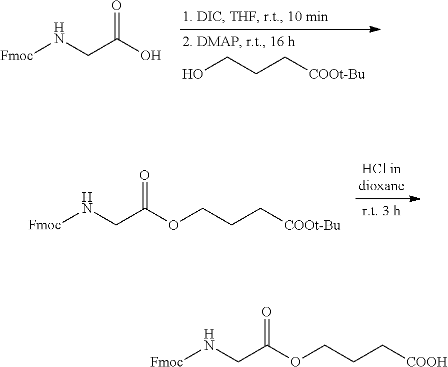

A mixture of Fmoc-Gly-OH (2.45 g, 8.25 mmol) and DIC (1.04 g, 8.25 mmol) in THF (15 mL) was stirred at room temperature (r.t.) for 10 min before the addition of DMAP (67 mg, 0.55 mmol) and tert-Butyl 4-hydroxybutanoate (880 mg, 5.5 mmol). The resulting mixture was stirred at r.t. overnight. The solvent was removed by rotatory evaporator, and the crude product was extracted with ethyl acetate (EA) (50 mL×3). The combined organic layers were washed by brine and dried over Na₂SO₄, concentrated and purified by the flash chromatography to afford the ester intermediate (1.6 g, 75% yield) as a white solid. The ester intermediate (1.6 g, 3.65 mmol) was dissolved in HCl/dioxane (2.5 M,5 mL) and stirred at r.t. for 3 h. The resulting crude product was purified by preparative reverse phase HPLC to afford Fmoc protected ESL1 carboxylic acid (800 mg) as a white solid.

### 1.7 LC-MS Condition

Instrument: Agilent prime-6125B_2LCMS
Column: Boltimate EXT C18 CoreShell 4.6×50 mm, 2.7 μm
Detection: UV (254 nm 214 nm 280 nm) and MS (ESI, 100 to 2000 amu)
Mobile Phase: A: H₂O (0.05% formic acid); B: ACN (0.05% formic acid)
Flow Rate: 2.0 mL/min
Column Temperature: 45° C.
Gradient: 10% to 95% B within 1.5 min, followed by 95% B for 1.0 min

### 1.8 Analytical HPLC Condition

Instrument: WATERS ARC UPLC
Column: XBridge BEH peptide BEH C18, 3.5 m, 2.1 mm×150 mm
Detection: UV 254 nm, 214 nm, 280 0 nm
Mobile Phase: A: H₂O (0.1% o TFA); B: ACN (0.1% TFA)
Column Temperature: 40° C.
Flow Rate: 0.6 mL/min
Gradient:

| | | | | | | |
|---|---|---|---|---|---|---|
| Time (min) | 0 | 1 | 11 | 13 | 13.5 | 15 |
| A % | 90 | 90 | 5 | 5 | 90 | 90 |
| B % | 10 | 10 | 95 | 95 | 10 | 10 |

The compounds of the present application, for example, the compounds as listed in Table 2 above, can be prepared using the general method as described. Exemplary compounds of the present application were synthesized and characterized as shown in Table 4 below.

TABLE 4
Synthesis and characterization of exemplary compounds of Formula (I-X)

| Com- | | Calculated | | Purity |
|---|---|---|---|---|
| pound | | Ave. | | (214 |
| No. | Sequence/Structure | MW | LC-MS | nm) |
| Reference | DOTAGA-Lys(PEG3-MAL)-Tyr-NH2 | 1050.1 | (M + H) +: 1050.4; | >99.0%. |
| | | | (M + 2H)2+: 525.8; | |
| L001 | HO-C18-gGlu-OEG-OEG-ESL1- | 2037.3 | (M + 2H)2+: 1019.4; | 93.0% |
| | Lys(DOTAGA)-Lys(PEG3-MAL)-Tyr- | | (M + 3H)3+: 679.8 | |
| | NH2 | | | |
| L002 | HO-C18-gGlu-OEG-OEG-Lys(ESL1- | 2037.3 | (M + 2H)2+: 1019.4; | >99.0% |
| | DOTAGA)-Lys(PEG3-MAL)-Tyr-NH2 | | (M + 3H)3+: 679.8 | |
| L003 | HO-C20-gGlu-OEG-OEG-OEG-OEG- | 2646.0 | (M + 2H)2+: 1323.6; | >99.0% |
| | OEG-OEG-ESL1-Lys(DOTAGA)- | | (M + 3H)3+: 882.8; | |
| | Lys(PEG3-MAL)-Tyr-NH2 | | (M + 4H)4+: 662.4 | |
| L004 | HO-C20-gGlu-OEG-OEG-OEG-OEG- | 2646.0 | (M + 2H)2+: 1323.6; | >99.0% |
| | OEG-OEG-Lys(ESL1-DOTAGA)- | | (M + 3H)3+: 882.7; | |
| | Lys(PEG3-MAL)-Tyr-NH2 | | (M + 4H)4+: 662.4 | |
| L005 | HO-C18-gGlu-OEG-OEG-OEG- | 2346.7 | (M + 2H)2+: 1174.0; | >99.0% |
| | Lys(ESL1-DOTAGA)-OEG-OEG-OEG- | | (M + 3H)3+: 782.9; | |
| | Lys[CO-CH2-C6H4-(p-NCS)]-NH2 | | (M + 4H)4+: 587.6 | |
| L006 | IPhBu-gGlu-PEG8-ESL1-Lys(DOTAGA)- | 2146.1 | (M + 2H)2+: 1073.8; | >99.0% |
| | Lys(PEG3-MAL)-Tyr-NH2 | | (M + 3H)3+: 716.3 | |
| L007 | IPhBu-gGlu-PEG8-Lys(ESL1-DOTAGA)- | 2146.1 | (M + 2H)2+: 1073.6; | >99.0% |
| | Lys(PEG3-MAL)-Tyr-NH2 | | (M + 3H)3+: 716.2 | |
| L012 | HO-C18-gGlu-OEG-OEG-Lys(C14)- | 2375.9 | (M + 2H)2+: 1188.4; | >96.6% |
| | ESL1-Lys(DOTAGA)-Lys(PEG3-MAL)- | | (M + 3H)3+: 792.7; | |
| | Tyr-NH2 | | (M + 4H)4+: 594.8 | |
| L013 | HO-C18-gGlu-OEG-OEG]2-Lys-ESL1- | 2881.4 | (M + 2H)2+: 1441.2; | >99.0% |
| | Lys(DOTAGA)-Lys(PEG3-MAL)-Tyr- | | (M + 3H)3+: 961.2; | |
| | NH2 | | (M + 4H)4+: 721.2 | |
| L013 | [HO-C18-gGlu-PEG8-PEG8]2-Lys-ESL1- | 3994.8 | 1132.0(M + 3H)3+, | 96.8% |
| | Lys(DOTAGA)-Lys(PEG3-MAL)-Tyr- | | 999.3(M + 4H)4+, | |
| | NH2 | | 799.7(M + 5H)5+, | |
| | | | 666.8(M + 6H)6+ | |
| L030 | [HO-C18-gGlu-gGlu-gGlu-OEG-OEG]2- | 3397.8 | 1133.1(M + 3H)3+, | >99.0% |
| | Lys-ESL1-Lys(DOTAGA)-Lys(PEG3- | | 850.2(M + 4H)4+ | |
| | MAL)-Tyr-NH2 | | | |
| L031 | [HO-C16-gGlu-OEG-OEG]2-Lys-ESL1- | 2825.3 | 1413.1(M + 2H)2+, | >99.0% |
| | Lys(DOTAGA)-Lys(PEG3-MAL)-Tyr- | | 942.3(M + 3H)4+, | |
| | NH2 | | 707.0(M + 4H)4+ | |
| L032 | [HO-C18-gGlu-PEG8-PEG8]2-Lys- | 3994.8 | 1332.4(M + 3H)3+, | 94.1% |
| | Lys(ESL1-DOTAGA)-Lys(PEG3-MAL)- | | 999.4c(M + 4H)4+, | |
| | Tyr-NH2 | | 799.8(M + 5H)5+, | |
| | | | 666.6(M + 6H)6+ | |
| L033 | [HO-C16-gGlu-PEG8-PEG8]2-Lys- | 3938.7 | 1320.8(M + | 90.3% |
| | Lys(ESL1-DOTAGA)-Lys(PEG3-MAL)- | | 2H + Na)3+, | |
| | Tyr-NH2 | | 985.4 (M + 4H)4+, | |
| | | | 788.6(M + 5H)5+, | |
| | | | 657.4(M + 6H)6+ | |
| L034 | [HO-C14-gGlu-PEG8-PEG8]2-Lys- | 3882.6 | 1941.4(M + 2H)2+, | 91.4% |
| | Lys(ESL1-DOTAGA)-Lys(PEG3-MAL)- | | 1294.9(M + 3H)3+, | |
| | Tyr-NH2 | | 777.3(M + 4H)4+, | |
| | | | 648.0(M + 5H)5+ | |
| L035 | [HO-C18-gGlu-PEG8-PEG8]2-Lys- | 4137.9 | 1379.7(M + 3H)3+, | 90.5% |
| | Lys(ESL1-ESL1-DOTAGA)-Lys(PEG3- | | 1035.1(M + 4H)4+, | |
| | MAL)-Tyr-NH2 | | 828.3(M + 5H)5+, | |
| | | | 690.6(M + 6H)6+ | |
| L036 | [HO-C18-gGlu-PEG8-PEG8-PEG8- | 5688.8 | 1138.3(M + 5H)5+, | 96.5% |
| | PEG8]2-Lys-Lys(ESL1-DOTAGA)- | | 948.8(M + 6H)6+, | |
| | Lys(PEG3-MAL)-Tyr-NH2 | | 813.4(M + 7H)7+, | |
| | | | 712.0(M + 8H)8+ | |
| L037 | HO-C18-gGlu-PEG8-PEG8-Lys(C12)- | 3880.6 | 1301.3(M + | 90.7% |
| | Glu-PEG8-PEG8-Lys(ESL1-DOTAGA)- | | 2H + Na)3+, | |
| | Lys(PEG3-MAL)-Tyr-NH2 | | 970.8(M + 4H)4+, | |
| | | | 776.8(M + 5H)5+, | |
| | | | 647.6(M + 6H)6+ | |
| L038 | HO-C18-gGlu-PEG8-PEG8-Lys(C12- | 3910.6 | 1303.9(M + 3H)3+, | 93.2% |
| | OH)-Glu-PEG8-PEG8-Lys(ESL1- | | 978.2(M + 4H)4+, | |
| | DOTAGA)-Lys(PEG3-MAL)-Tyr-NH2 | | 782.8(M + 5H)5+, | |
| | | | 652.7(M + 6H)6+ | |
| L039 | HO-C18-gGlu-PEG8-Nal-PEG8-Nal- | 3412 | 1706.2(M + 2H)2+, | 94.3% |
| | PEG8-Lys(ESL1-DOTAGA)-Lys(PEG3- | | 1138.1(M + 3H)3+, | |
| | MAL)-Tyr-NH2 | | 853.8(M + 4H)4+, | |
| | | | 683.2(M + 5H)5+ | |
| L040 | HO-C18-gGlu-PEG8-PEG8-Lys(HO- | 3966.7 | 1322.8(M + 3H)3+, | 95.1% |
| | C16)-Glu-PEG8-PEG8-Lys(ESL1- | | 992.3(M + 4H)4+, | |
| | DOTAGA)-Lys(PEG3-MAL)-Tyr-NH2 | | 794.2(M + 5H)5+, | |
| | | | 662.0(M + 6H)6+ | |
| L041 | [HO-C18-gGlu-PEG8-PEG8-PEG8- | 5980 | 1196.3(M + 5H)5+, | 93.8% |
| | PEG8]2-Lys-Lys(OEG-ESL1-OEG- | | 997.2(M + 6H)6+, | |
| | DOTAGA)-Lys(PEG3-MAL)-Tyr-NH2 | | 854.8(M + 7H)7+, | |
| | | | 748.2(M + 8H)8+ | |
| L042 | HO-C18-gGlu-PEG12-PEG12-Glu- | 4770.7 | 1590.9(M + 3H)3+, | 100.0% |
| | Lys(C16)-Glu-PEG12-PEG12-Lys(ESL1- | | 1198.9(M + 4H)4+, | |
| | DOTAGA)-Lys(PEG3-MAL)-Tyr-NH2 | | 955.0(M + 5H)5+, | |
| L043 | [HO-C17-gGlu-PEG8-PEG8-PEG8- | 5660.7 | 1132.6(M + 5H)5+, | 94.90% |
| | PEG8]2-Lys-Lys(ESL1-DOTAGA)- | | 944.0(M + 6H)6+, | |
| | Lys(PEG3-MAL)-Tyr-NH2 | | 809.5(M + 7H)7+, | |
| | | | 708.4(M + 8H)8+ | |
| L044 | HO-C20-gGlu-PEG12-PEG12-PEG12- | 5373.4 | 1075.3(M + 5H)5+, | >99.0% |
| | PEG12-PEG12-PEG12-Lys(ESL1- | | 896.3(M + 6H)6+, | |
| | DOTAGA)-Lys(PEG3-MAL)-Tyr-NH2 | | 768.4(M + 7H)7+, | |
| | | | 768.4(M + 8H)8+ | |
| L045 | [HO-C18-gGlu]2-Lys-PEG12-PEG12- | 5899 | 1180.4(M + 5H)5+, | 96.60% |
| | PEG12-PEG12-PEG12-PEG12-Lys(ESL1- | | 983.8(M + 6H)6+, | |
| | DOTAGA)-Lys(PEG3-MAL)-Tyr-NH2 | | 843.5(M + 7H)7+, | |
| | | | 738.2(M + 8H)8+ | |
| L046 | [HO-C18-gGlu-PEG8-PEG8-PEG8- | 6269.4 | 1567.9 (M + 3H)3+, | >99.0% |
| | PEG8]2-Lys-Lys(OEG-OEG-ESL1-OEG- | | 1254.4(M + 5H)5+, | |
| | OEG-DOTAGA)-Lys(PEG3-MAL)-Tyr- | | 1045.7(M + 6H)6+, | |
| | NH2 | | 896.5(M + 7H)7+, | |
| L047 | [HO-C18-gGlu-PEG8-PEG8-PEG8- | 5545.6 | 1109.6(M + 5H)5+, | >99.0% |
| | PEG8]2-Lys-Lys(DOTAGA)-Lys(PEG3- | | 924.8(M + 6H)6+, | |
| | MAL)-Tyr-NH2 | | 793.0(M + 7H)7+, | |
| | | | 694.0(M + 8H)8+ | |
| L048 | HO-C20-gGlu-PEG8-PEG8-PEG8-PEG8- | 3325.9 | 1109.6(M + 3H)3+, | >99.0% |
| | Lys(DOTAGA)-Lys(PEG3-MAL)-Tyr- | | 832.2(M + 4H)4+, | |
| | NH2 | | 666.0(M + 7H)7+, | |
| | | | 555.2(M + 8H)8+ | |
| L049 | HO-C18-gGlu-PEG8-PEG8-PEG8-PEG8- | 3297.9 | 1099.8 (M + 3H)3+, | >99.0% |
| | Lys(DOTAGA)-Lys(PEG3-MAL)-Tyr- | | 825.2(M + 4H)4+, | |
| | NH2 | | 660.4(M + 7H)7+, | |
| | | | 550.6(M + 8H)8+ | |
| L060 | [HO-C18-gGlu-PEG8-PEG8-PEG8- | 6535.8 | 934.2(M + 7H)⁷⁺; | >99.0% |
| | PEG8]2-Lys-Lys(PEG8-ESL1-PEG8- | | 817.8(M + 8H)⁸⁺; | |
| | DOTAGA)-Lys(PEG3-MAL)-Tyr-NH2 | | 727.0(M + 9H)⁹⁺ | |
| L061 | HO-C20-gGlu-PEG8-PEG8-PEG8-PEG8- | 4316.1 | 1439.1(M + 3H)³⁺; | 95.2% |
| | Lys(PEG8-ESL1-PEG8-DOTAGA)- | | 1079. 6(M + 4H)⁴⁺; | |
| | Lys(PEG3-MAL)-Tyr-NH2 | | 863.8(M + 5H)⁵⁺ | |
| L062 | HO-C18-gGlu-PEG8-PEG8-PEG8-PEG8- | 3441.0 | 1147.5(M + 3H)³⁺; | >99.0% |
| | Lys(ESL1-DOTAGA)-Lys(PEG3-MAL)- | | 860.8(M + 4H)⁴⁺; | |
| | Tyr-NH2 | | 689.0(M + 5H)⁵⁺ | |
| L063 | HO-C20-gGlu-PEG8-PEG8-PEG8-PEG8- | 3469.1 | 1156.9(M + 3H)³⁺; | >99.0% |
| | ESL1-Lys(DOTAGA)-Lys(PEG3-MAL)- | | 868.0 (M + 4H)⁴⁺; | |
| | Tyr-NH2 | | 694.7(M + 5H)⁵⁺ | |
| L064 | HO-C20-gGlu-PEG8-PEG8-PEG8-PEG8- | 3469.1 | 1156.8(M + 3H)³⁺; | >99.0% |
| | Lys(ESL1-DOTAGA)-Lys(PEG3-MAL)- | | 867.9 (M + 4H)⁴⁺; | |
| | Tyr-NH2 | | 694.6(M + 5H)⁵⁺ | |
| L097 | HO-C18-gGlu-PEG8-PEG8-PEG8-PEG8- | 3441.0 | 1147.4(M + 3H)³⁺; | >99.0% |
| | ESL1-Lys(DOTAGA)-Lys(PEG3-MAL)- | | 860.9(M + 4H)⁴⁺; | |
| | Tyr-NH2 | | 689.0(M + 5H)⁵⁺ | |
| L066 | HO-C20-gGlu-PEG8-PEG8-PEG8-PEG8- | 3483.1 | 1161.4(M + 3H)³⁺; | >99.0% |
| | Lys(5hPA-Gly-DOTAGA)-Lys(PEG3- | | 871.4 (M + 4H)⁴⁺; | |
| | MAL)-Tyr-NH2 | | 697.5(M + 5H)⁵⁺ | |
| L067 | HO-C20-gGlu-PEG8-PEG8-PEG8-PEG8- | 3497.1 | 1166.2(M + 3H)³⁺; | >99.0% |
| | Lys(6hHA-Gly-DOTAGA)-Lys(PEG3- | | 875.0(M + 4H)⁴⁺; | |
| | MAL)-Tyr-NH2 | | 700.3(M + 5H)⁵⁺ | |
| L068 | [HO-C18-gGlu-PEG8-PEG8-PEG8- | 6537.8 | 1090.4(M + 6H)⁶⁺; | >99.0% |
| | PEG8]2-Lys-Lys(PEG8-OEG-PEG8- | | 934.6(M + 7H)⁷⁺; | |
| | DOTAGA)-Lys(PEG3-MAL)-Tyr-NH2 | | 818.0(M + 8H)⁸⁺ | |
| L069 | HO-C20-gGlu-PEG8-PEG8-PEG8-PEG8- | 3804.5 | 1902.8(M + 2H)²⁺; | >99.0% |
| | Lys(PEG6-ESL1-DOTAGA)-Lys(PEG3- | | 1268.8(M + 3H)³⁺; | |
| | MAL)-Tyr-NH2 | | 951.8(M + 4H)⁴⁺ | |
| L070 | HO-C20-gGlu-PEG8-PEG8-PEG8-PEG8- | 4037.7 | 1346.3(M + 3H)³⁺; | >99.0% |
| | Lys(PEG8-ESL1-OEG-DOTAGA)- | | 1010.0(M + 4H)⁴⁺; | |
| | Lys(PEG3-MAL)-Tyr-NH2 | | 808.2(M + 5H)⁵⁺ | |
| L071 | HO-C20-gGlu-PEG8-PEG8-PEG8-PEG8- | 3949.6 | 988.0(M + 4H)⁴⁺; | >99.0% |
| | Lys(PEG6-ESL1-OEG-DOTAGA)- | | 790.6(M + 5H)⁵⁺; | |
| | Lys(PEG3-MAL)-Tyr-NH2 | | 659.2(M + 6H)⁶⁺ | |
| L072 | HO-C20-gGlu-PEG8-PEG8-PEG8-PEG8- | 4139.9 | 1380.3(M + 3H)³⁺; | >99.0% |
| | Lys(PEG8-ESL1-PEG4-DOTAGA)- | | 1035. 7(M + 4H)⁴⁺; | |
| | Lys(PEG3-MAL)-Tyr-NH2 | | 828.6(M + 5H)⁵⁺ | |
| L073 | HO-C20-gGlu-PEG8-PEG8-PEG8-PEG8- | 4183.9 | 1395.0(M + 3H)³⁺; | 99.5% |
| | Lys(PEG8-ESL1-PEG5-DOTAGA)- | | 1046.4(M + 4H)⁴⁺; | |
| | Lys(PEG3-MAL)-Tyr-NH2 | | 837.4(M + 5H)⁵⁺ | |
| L074 | HO-C20-gGlu-PEG8-PEG8-PEG8-PEG8- | 4228.0 | 1409.7(M + 3H)³⁺; | >99.0% |
| | Lys(PEG8-ESL1-PEG6-DOTAGA)- | | 1057.7(M + 4H)⁴⁺; | |
| | Lys(PEG3-MAL)-Tyr-NH2 | | 846.4(M + 5H)⁵⁺ | |
| L075 | HO-C20-gGlu-PEG8-PEG8-PEG8-PEG8- | 4272.0 | 1424.3(M + 3H)³⁺; | 98.2% |
| | Lys(PEG8-ESL1-PEG7-DOTAGA)- | | 1068.6(M + 4H)⁴⁺; | |
| | Lys(PEG3-MAL)-Tyr-NH2 | | 855.0(M + 5H)⁵⁺ | |
| L077 | HO-C20-gGlu-PEG8-PEG8-PEG8-PEG8- | 4199.0 | 1400.1(M + 3H)³⁺; | 94.9% |
| | Lys(PEG8-6hHA-Gly-PEG6-DOTAGA)- | | 840.4 (M + 5H)⁵⁺; | |
| | Lys(PEG3-MAL)-Tyr-NH2 | | 700.6(M + 6H)⁶⁺ | |
| L078 | HO-C20-gGlu-PEG8-PEG8-PEG8-PEG8- | 4318.1 | 1439.8(M + 3H)³⁺; | >99.0% |
| | Lys(PEG8-OEG-PEG8-DOTAGA)- | | 864.4(M + 5H)⁵⁺; | |
| | Lys(PEG3-MAL)-Tyr-NH2 | | 720.5(M + 6H)⁶⁺ | |
| L096 | [HO-C18-gGlu-PEG8-PEG8]2-Lys- | 4841.8 | 1614.1(M + 3H)³⁺; | >93.15 |
| | Lys(PEG8-ESL1-PEG8-DOTAGA)- | | 969.0(M + 5H)⁵⁺; | |
| | Lys(PEG3-MAL)-Tyr-NH2 | | 807.7(M + 6H)⁶⁺ | |
| L098 | [HO-C18-gGlu-PEG8-PEG8-PEG8- | 5917.0 | 986.8(M + 6H)⁶⁺; | >99.9% |
| | PEG8]2-Lys-Lys(Gly-Gly-ESL1-Gly-Gly- | | 846.0(M+7H)⁷⁺; | |
| | DOTAGA)-Lys(PEG3-MAL)-Tyr-NH2 | | 740.5(M + 8H)⁸⁺ | |

### Example 2 Preparation of the Exemplary sdAb Conjugates of the Present Application

Anti-Claudin 18.2 sdAbs, anti-GCC sdAb, and anti-FAP sdAb were used as moiety B, respectively, to prepare the exemplary conjugates of the present application. Briefly, 5 DNA sequence encoding each of CLDN18.2C(S) (SEQ ID NO. 9), GCCC(S); GCCC(Q); GCCC(P); GCCC(H) (SEQ ID NO. 14-17) and FAPC(S) (SEQ ID NO. 22) was cloned into pcDNA3.4 vector, followed by transient transfection into CHO-K1 cells for expression of the target binding moiety. The expressed target binding moiety in supernatant growth medium was then purified by Amsphere A3 resin and then validated by LC-MS.

The target binding moiety was firstly treated with EDTA and TCEP solution for 1 hour at 37° C., followed by desalting to change the buffer to 100 mM PB buffer, pH 7.0 via a Cytiva PD-10 pre-packed column. Then approximately 10 eq. of each compound was dissolved in DMSO and then transferred into the desalted targeting moiety solution for conjugation at 37° C. for 1 hour. The conjugate then was purified via an Amicon ultrafiltration filter unit to remove unconjugated compound and to exchange buffer to DPBS.

The conjugate was further subject to LC-MS analysis. The DOL (Degree of Ligation) was calculated based on the LC-MS results.

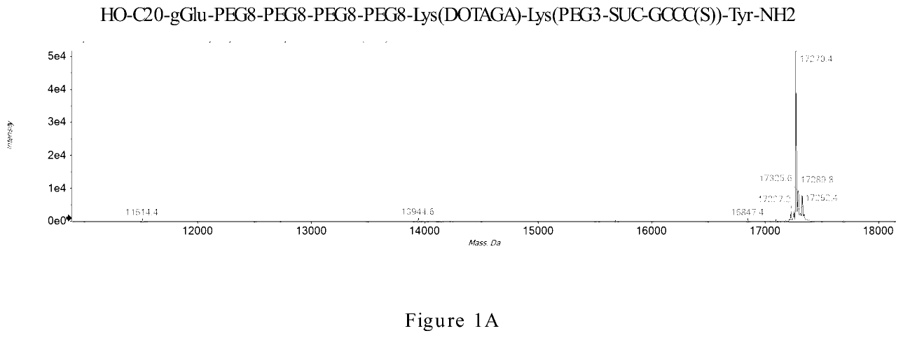

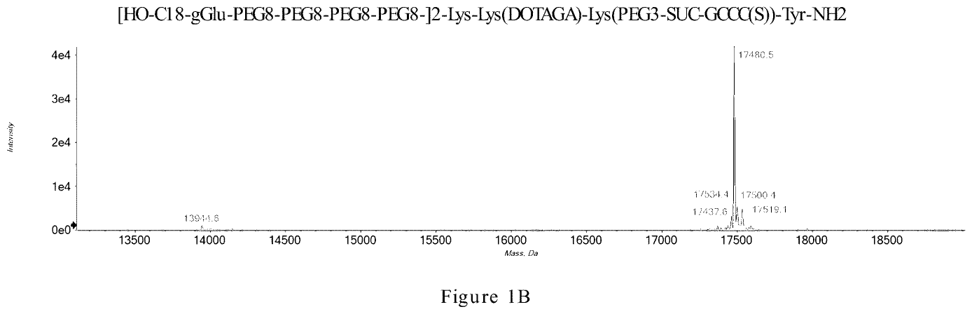

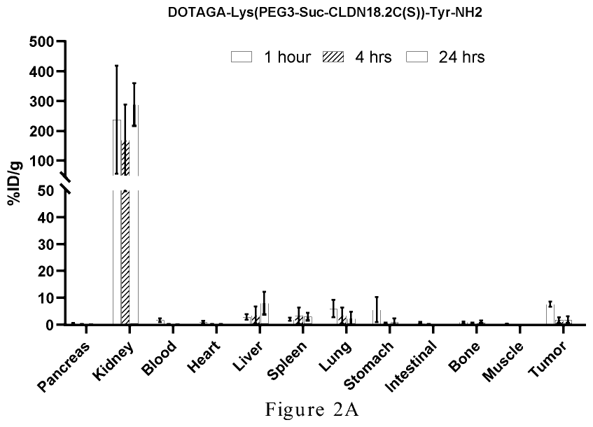

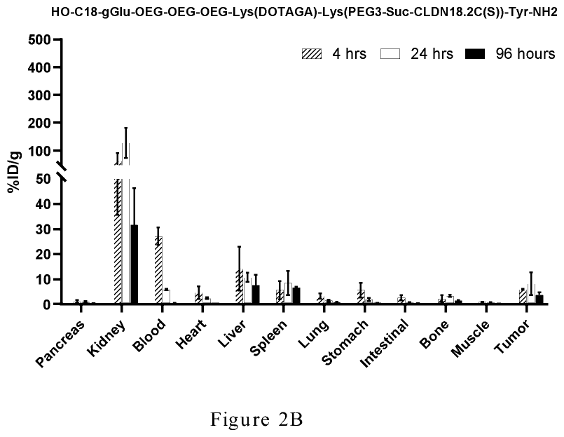

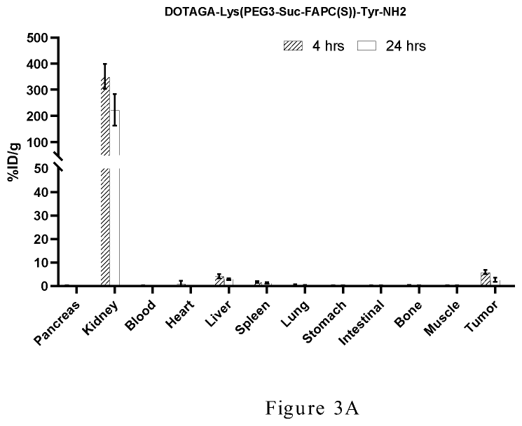

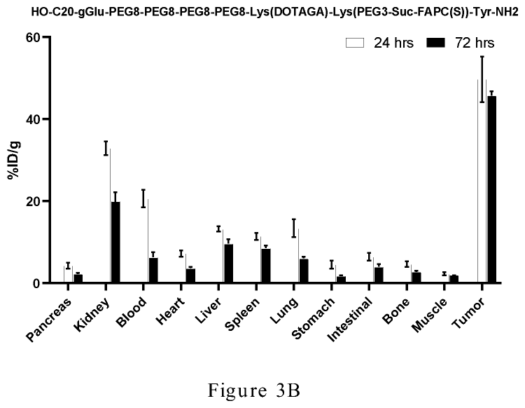

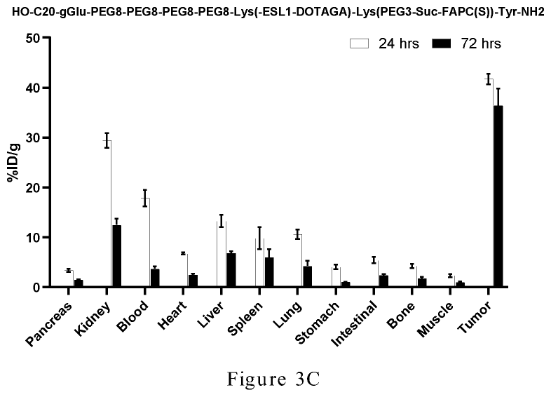

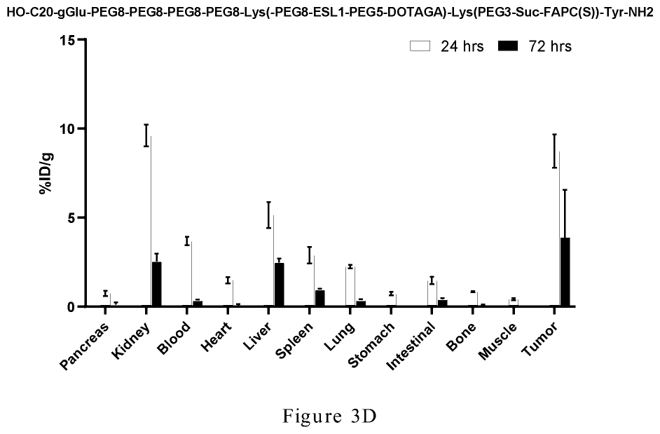

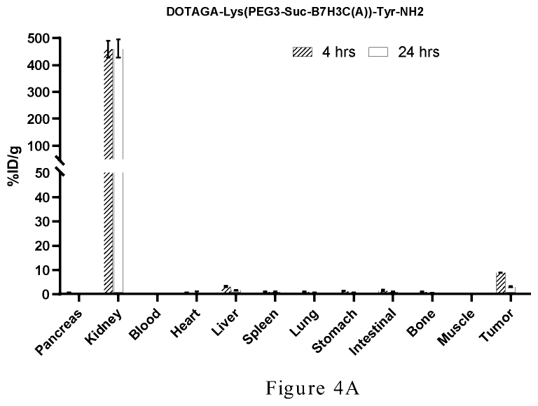

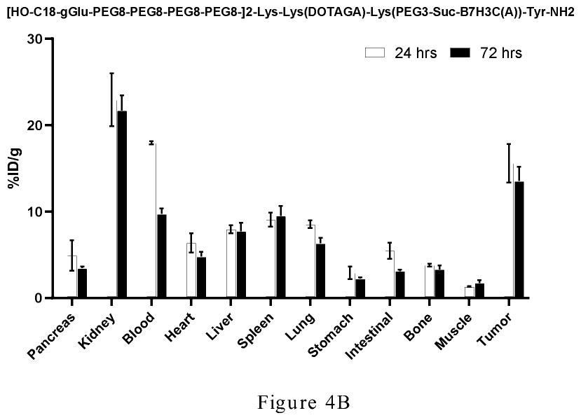

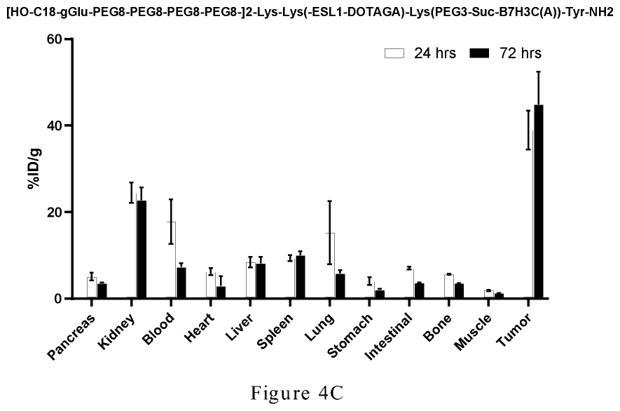

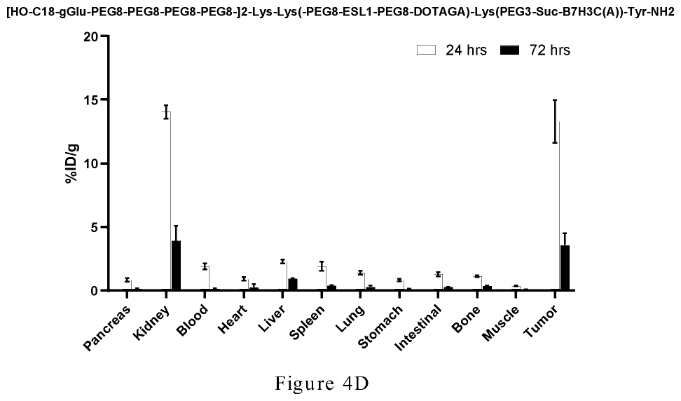

FIG. 1 exemplarily show the LS-MS results after conjugation. As seen in FIG. 1, the compounds I-01 and I-02 were successfully conjugated to via the Cys residue at C terminus of CLDN18.2C(S) in a site-specific manner with a DOL number of 1.0.

### Example 3. Binding Affinities of the Exemplary sdAb Conjugates with the Targets Thereof

The binding affinities of Claudin 18.2 sdAb conjugates were determined by ELISA. 100 μL DPBS containing 200 ng of Claudin 18.2 VLP protein (KactusBio, Cat #: CLD-HE1822) was coated on a 96-well plate (Nunc, Cat #: 442404) per well overnight at 4° C. On the next day, after washing four times with 0.05% PBST (ThermoFisher, Cat #: 28352) buffer, the plate was blocked by 10% StartingBlock PBS (ThermoFisher, Cat #: 37538) blocking buffer diluted in 0.05% PBST buffer for 2 hours at room temperature. The plate was then washed four times using 0.05% PBST buffer, added 100 μL per well of sample sdAb conjugates serially diluted in 0.05% PBST containing 1% StartingBlock buffer, and incubated on a 100-rpm orbital shaker for 2 hours at room temperature. After the incubation, the plate was washed four times using 0.05% PBST buffer, added 100 μL per well of 0.4 μg/mL anti-VHH secondary antibody, HRP (Jackson, Cat #: 128-035-232) diluted in 0.05% PBST containing 1% StartingBlock buffer, and incubated on a 100-rpm orbital shaker for 1 hours at room temperature. After the incubation, the plate was washed four times using 0.05% PBST buffer, developed using 100 μL per well of TMB-ELISA substrate solution (KPL, Cat #: 5120-00820) for 6 min at room temperature, and stopped using 100 μL per well of stop solution (Beyotime, Cat #: P0215-500 mL). The optical density at 450 nm and 570 nm per well was then read by a plate reader (Molecular Devices, SpectraMax M5), wherein the readouts were used for EC₅₀ calculation in Graphpad Prism 9.3.1 using variable slope (four parameters) model. The calculated EC₅₀ results are shown in Table 5.

The EC₅₀ data suggested that the sdAb and their conjugates can bind to the respective targeting receptor over-expressed cell lines with EC₅₀ values in the range of 0.6-4.7 nM. The linkers appended at the C-terminus of the sdAb had minimal impact on its binding affinity.

The binding affinities of GUCY2C and FAP sdAb conjugates were determined by flow cytometry based competitive binding assays using HEK293F cells that overexpress human GUCY2C, and HEK293T cells that overexpress human FAP, respectively. A competitive Alexa Fluor 488 labelled anti-GUCY2C sdAb (SEQ ID No. 10), and a monovalent single-chain Fc (scFc) tagged anti-FAP sdAb (SEQ ID No. 18; tag-excluded) were applied respectively as a competitor against the corresponding sdAb conjugates.

For GUCY2C assay, the HEK293F cells overexpressing GUCY2C were harvested through standard Trypsin-EDTA treatment followed by a spin at 400 g for 4 min, wherein the pelleted cells were then re-suspended in FACS Buffer A (DMEN containing 1% OVA, Admas, Cat #: 89204J) at a density of 2×10⁶ cells/mL. 50 μL of the re-suspended cells, 50 μL of Alexa Fluor 488 labelled anti-GUCY2C sdAb in FACS Buffer A (working concentration of 10 nM), and a serial dilute of GUCY2C sdAb conjugates were mixed thoroughly in a V-bottom 96-well plate. The mixture was then incubated on an orbital shaker at 100 rpm at RT for 1 hour, protecting from light. Unbound Alexa Fluor 488 labelled anti-GUCY2C sdAbs were removed by washing the plate three times with DPBS containing 1% OVA, wherein the plate was then measured for fluorescence intensity via a NovoCyte flow cytometer. IC₅₀values based on normalized MFI were calculated by using GraphPad Prism 10, wherein MFI from 10 nM Alexa Fluor 488 labelled anti-GUCY2C sdAb wells were deemed as HC and MFI of cell-only wells were deemed as LC.

For FAP assay, the HEK293T cells overexpressing FAP were harvested through standard Trypsin-EDTA treatment followed by a spin at 400 g for 4 min, wherein the pelleted cells were then re-suspended in FACS Buffer B (DMEM containing 10% protein-free blocking buffer, ThermoFisher, Cat #: 37572) at a density of 2×10⁶ cells/mL. A serial dilute of FAP sdAb conjugates was added to 50 μL of the re-suspended cells, wherein the plate was then incubated at 4° C. for 120 min. After washing the plate twice with FACS Buffer B, 100 μL of the scFc-tagged anti-FAP sdAb in FACS Buffer B (working concentration of 3 nM) was added per well for competition, wherein the plate was incubated at 4° C. for 30 min. After washing the plate twice with DPBS containing 1% OVA, Alexa Fluor® 647 AffmiPure Goat Anti-Human IgG secondary antibody (Jackson, Cat #: 109-605-098) with working concentration of 1.5 μg/mL was added per well, wherein the plate was incubated at 4° C. for 60 min, protecting from light. Unbound fluorescent secondary antibodies were then removed by washing the plate twice with FACS Buffer B, wherein the plate was fixed by 2% paraformaldehyde and then measured for fluorescence intensity via a NovoCyte flow cytometer. IC₅₀values based on direct MFI readouts were calculated by using GraphPad Prism 10, wherein an anti-FAP sdAb (SEQ ID NO. 18) was applied as a positive control, and an anti-Claudin 18.2 sdAb was applied as a negative control. The calculated IC₅₀results are shown in Table 5.

The IC₅₀data suggested that the sdAb and their conjugates can bind to the respective targeting receptor over-expressed cell lines with IC₅₀values in the range of 5-25 nM. The linkers appended at the C-terminus of the sdAb had minimal impact on its binding affinity.

TABLE 5
Binding affinities of the exemplary conjugates of the present application

| Sequence/Structure | IC₅₀ (nM) |
|---|---|
| GCC (SEQ ID NO. 10) | 17.4 |
| GCCC(S) (SEQ ID NO. 14) | 15.0 |
| GCCC(Q) (SEQ ID NO. 15) | 24.4 |
| GCCC(P) (SEQ ID NO. 16) | 21.3 |
| GCCC(H) (SEQ ID NO. 17) | 22.0 |
| DOTAGA-Lys(PEG3-Suc-GCCC(S))-Tyr-NH2 | 19.5 |
| HO-C20-gGlu-OEG-OEG-OEG-OEG-OEG-OEG-Lys(ESL1-DOTAGA)-Lys(PEG3-Suc- | 7.41 |
| GCCC(S))-Tyr-NH2 | |
| [HO-C18-gGlu-PEG8-PEG8]2-Lys-ESL1-Lys(DOTAGA)-Lys(PEG3-Suc-GCCC(S))-Tyr- | 15.5 |
| NH2 | |
| [HO-C18-gGlu-gGlu-gGlu-OEG-OEG]2-Lys-ESL1-Lys(DOTAGA)-Lys(PEG3-Suc- | 15.6 |
| GCCC(S))-Tyr-NH2 | |
| [HO-C18-gGlu-PEG8-PEG8]2-Lys-Lys(ESL1-DOTAGA)-Lys(PEG3-Suc-GCCC(S))-Tyr- | 11.0 |
| NH2 | |
| [HO-C18-gGlu-PEG8-PEG8]2-Lys-Lys(ESL1-ESL1-DOTAGA)-Lys(PEG3-Suc- | 11.8 |
| GCCC(S))-Tyr-NH2 | |
| [HO-C18-gGlu-PEG8-PEG8-PEG8-PEG8]2-Lys-Lys(ESL1-DOTAGA)-Lys(PEG3-Suc- | 13.1 |
| GCCC(S))-Tyr-NH2 | |
| HO-C18-gGlu-PEG8-PEG8-Lys(C12)-Glu-PEG8-PEG8-Lys(ESL1-DOTAGA)-Lys(PEG3- | 8.64 |
| Suc-GCCC(S))-Tyr-NH2 | |
| HO-C18-gGlu-PEG8-Nal-PEG8-Nal-PEG8-Lys(ESL1-DOTAGA)-Lys(PEG3-Suc- | 11.1 |
| GCCC(S))-Tyr-NH2 | |
| HO-C18-gGlu-PEG8-PEG8-Lys(HO-C16)-Glu-PEG8-PEG8-Lys(ESL1-DOTAGA)- | 10.9 |
| Lys(PEG3-Suc-GCCC(S))-Tyr-NH2 | |
| [HO-C17-gGlu-PEG8-PEG8-PEG8-PEG8]2-Lys-Lys(ESL1-DOTAGA)-Lys(PEG3-Suc- | 10.2 |
| GCCC(S))-Tyr-NH2 | |
| HO-C20-gGlu-PEG12-PEG12-PEG12-PEG12-PEG12-PEG12-Lys(ESL1-DOTAGA)- | 6.68 |
| Lys(PEG3-Suc-GCCC(S))-Tyr-NH2 | |
| [HO-C18-gGlu]2-Lys-PEG12-PEG12-PEG12-PEG12-PEG12-PEG12-Lys(ESL1- | 10.9 |
| DOTAGA)-Lys(PEG3-Suc-GCCC(S))-Tyr-NH2 | |
| [HO-C18-gGlu-PEG8-PEG8-PEG8-PEG8]2-Lys-Lys(DOTAGA)-Lys(PEG3-Suc- | 6.37 |
| GCCC(S))-Tyr-NH2 | |
| HO-C20-gGlu-PEG8-PEG8-PEG8-PEG8-Lys(DOTAGA)-Lys(PEG3-Suc-GCCC(S))-Tyr- | 4.88 |
| NH2 | |
| HO-C18-gGlu-PEG8-PEG8-PEG8-PEG8-Lys(DOTAGA)-Lys(PEG3-Suc-GCCC(S))-Tyr- | 4.78 |
| NH2 | |
| FAPC(S)(SEQ ID NO. 22) | 13.4 |
| | (N = 4) |
| DOTAGA-Lys(PEG3-Suc-FAPC(S))-Tyr-NH2 | 21.6 |
| HO-C20-gGlu-PEG8-PEG8-PEG8-PEG8-Lys(DOTAGA)-Lys(PEG3-Suc-FAPC(S))-Tyr- | 22.2 |
| NH2 | |
| HO-C18-gGlu-PEG8-PEG8-PEG8-PEG8-Lys(DOTAGA)-Lys(PEG3-Suc-FAPC(S))-Tyr- | 25.3 |
| NH2 | |
| HO-C18-gGlu-PEG8-PEG8-PEG8-PEG8-Lys(ESL1-DOTAGA)-Lys(PEG3-Suc-FAPC(S))- | 24.6 |
| Tyr-NH2 | |
| HO-C20-gGlu-PEG8-PEG8-PEG8-PEG8-ESL1-Lys(DOTAGA)-Lys(PEG3-Suc-FAPC(S))- | 24.5 |
| Tyr-NH2 | |
| HO-C20-gGlu-PEG8-PEG8-PEG8-PEG8-Lys(ESL1-DOTAGA)-Lys(PEG3-Suc-FAPC(S))- | 24.8 |
| Tyr-NH2 | |
| HO-C20-gGlu-PEG8-PEG8-PEG8-PEG8-Lys(PEG8-ESL1-PEG8-DOTAGA)-Lys(PEG3- | 22.2 |
| Suc-FAPC(S))-Tyr-NH2 | |
| [HO-C18-gGlu-PEG8-PEG8-PEG8-PEG8]2-Lys-Lys(PEG8-ESL1-PEG8-DOTAGA)- | 25.2 |
| Lys(PEG3-Suc-FAPC(S))-Tyr-NH2 | |
| [HO-C18-gGlu-PEG8-PEG8-PEG8-PEG8]2-Lys-Lys(PEG8-OEG-PEG8-DOTAGA)- | 17.3 |
| Lys(Lys(PEG3-Suc-FAPC(S))-Tyr-NH2 | |
| HO-C20-gGlu-PEG8-PEG8-PEG8-PEG8-Lys(PEG8-ESL1-OEG-DOTAGA)- | 13.0 |
| LysLys(PEG3-Suc-FAPC(S))-Tyr-NH2 | |
| HO-C20-gGlu-PEG8-PEG8-PEG8-PEG8-Lys(6hHA-Gly-DOTAGA)-Lys(PEG3-Suc- | 16.3 |
| FAPC(S))-Tyr-NH2 | |
| HO-C20-gGlu-PEG8-PEG8-PEG8-PEG8-Lys(PEG8-ESL1-PEG5-DOTAGA)-Lys(PEG3- | 7.8 |
| Suc-FAPC(S))-Tyr-NH2 | |
| HO-C20-gGlu-PEG8-PEG8-PEG8-PEG8-Lys(PEG8-ESL1-PEG7-DOTAGA)-Lys(PEG3- | 9.3 |
| Suc-FAPC(S))-Tyr-NH2 | |
| HO-C20-gGlu-PEG8-PEG8-PEG8-PEG8-Lys(PEG8-6hHA-Gly-PEG6-DOTAGA)- | 11.0 |
| Lys(PEG3-Suc-FAPC(S))-Tyr-NH2 | |
| HO-C20-gGlu-PEG8-PEG8-PEG8-PEG8-Lys(PEG8-ESL1-PEG6-DOTAGA)-Lys(PEG3- | 9.9 |
| Suc-FAPC(S))-Tyr-NH2 | |
| HO-C20-gGlu-PEG8-PEG8-PEG8-PEG8-Lys(PEG8-ESL1-PEG4-DOTAGA)-Lys(PEG3- | 10.0 |
| Suc-FAPC(S))-Tyr-NH2 | |
| Sequence/Structure | EC₅₀ (nM) |
| CLDN18.2C(S)(SEQ ID NO. 9) | 0.5688 |
| DOTAGA-Lys(PEG3-Suc-CLDN18.2C(S))-Tyr-NH2 | 4.699 |
| HO-C18-gGlu-OEG-OEG-OEG-Lys(DOTAGA)-Lys(PEG3-Suc-CLDN18.2C(S))-Tyr-NH2 | 0.7937 |

### Example 4 Preparation of the Exemplary Affibody Conjugates of the Present Application

To B7H3 affibody (SEQ ID NO. 25, 10.0 mg, 1.50 μmol) in 6.0 mL of 50 mM PBS buffer (50 mM NaH2PO4, 300 mM NaCl, 40 mM imidazole, pH 7.5) were added TCEP (0.22 mg, 0.75 μmol) and urea (0.72 mg, 12.0 μmol). The mixture (pH ~7.0) was stirred at 37° C. for 30 min. Then each linker (19.6 mg, 3.00 μmol) dissolved in 2.0 mL of deionized water was added and the resulting mixture was stirred at 37° C. for 2 h.

Then conjugation mixture was loaded to a preparative HPLC column (Agilent ZORBAX RX-C8, 5 μm, 80 Å, 9.4×250 mm) for purification. The fractions within a desired purity range were pooled and lyophilized. The final purified affibody conjugate (2.0 mg, 97.200 pure) was obtained as a fluffy powder.

### Example 5 Binding Affinities of the Exemplary Affibody Conjugates with the Targets Thereof

The binding affinities of B7-H-3 affibody conjugates was determined by a flow cytometry based competitive binding assay using CT26 cells that overexpress human B7-H3 (Cobioer Biosciences, Cat #: CBPGO018) and a biotinylated B7H3 ligand (biotin conjugated SEQ ID NO. 25). The assay started with harvesting and centrifuging the CT26 cells at 400 g for 4 min, then re-suspended them in a FACS buffer (PBS containing 10% protein free blocking buffer, ThermoFisher, Cat #: 37572) at a density of 2*10⁶ cells/ml. The assay was set up by mixing 50 μL of cell suspension, 50 μL of biotinylated ligand solution (final concentration of 81 nM in the FACS buffer) and 50 μL of series diluted test affibody conjugate solution (in the FACS buffer) in a 96-well plate. A Her-2 affibody (SEQ ID NO. 26) was included as a negative control. The resulting plate was incubated at 4° C. for 2 h, then washed twice with the FACS buffer, followed by the addition of Alexa Fluor® 647-conjugated streptavidin (final concentration of 1.8 μg/ml, Jackson, Cat #: 016-600-084) and further incubation at 4° C. for 1 h. The plate was washed twice with the FACS buffer, and the resulting samples were measured for fluorescence intensity via a flow cytometer (NovoCyte Flow Cytom) and the IC₅₀values were calculated by using GraphPad Prism 10. The data is summarized in Table 6.

The IC₅₀data suggested that the B7-H3 affibody and their conjugates can specifically bind to the CT26 cells over-expressing huma B7H3 with IC₅₀values of 50-300 nM. The linkers appended at the C-terminus of the affibody had minor impact on the binding affinity, and such effect was non-distinguishable among different linkers.

TABLE 6
Binding affinities of the exemplary B7-H3
affibody conjugates of the present application

| Sequence/Structure | IC₅₀ (nM) |
|---|---|
| HER-2 affibody (SEQ ID NO. 26, negative control) | No binding |
| B7H3C(A)(SEQ ID NO. 25) | 55.5 (N = 1) |
| DOTAGA-Lys(PEG3-Suc-B7H3C(A))-Tyr-NH2 | 190 (N = 3) |
| HO-C20-gGlu-OEG-OEG-OEG-OEG-OEG-OEG- | 139 (N= 3) |
| Lys(ESL1-DOTAGA)-Lys(PEG3-Suc-B7H3C(A))- | |
| Tyr-NH2 | |
| [HO-C18-gGlu-PEG8-PEG8-PEG8-PEG8]2-Lys- | 213 (N= 1) |
| Lys(PEG8-ESL1-PEG8-DOTAGA)-Lys(PEG3-Suc- | |
| B7H3C(A))-Tyr-NH2 | |
| [HO-C18-gGlu-PEG8-PEG8-PEG8-PEG8]2-Lys- | 296 (N= 1) |
| Lys(ESL1-DOTAGA)-Lys(PEG3-Suc-B7H3C(A))- | |
| Tyr-NH2 | |
| [HO-C18-gGlu-PEG8-PEG8-PEG8-PEG8]2-Lys- | 232 (N= 1) |
| Lys(DOTAGA)-Lys(PEG3-Suc-B7H3C(A))-Tyr-NH2 | |

### Example 6 Biodistribution Study of the Exemplary Conjugates of the Present Application

The BALB/c nude mice (Charles River, Beijing) were utilized to establish the xenografted model for biodistribution studies. All animal care and experimental procedure were performed by following the animal protocols approved by the ethics committee of China Institute of Radiation Protection.

To evaluate the in vivo biodistribution of the sdAb conjugates targeting Claudin 18.2, each mouse was inoculated with approximately 1×10⁷ human Claudin 18.2 expressing HEK-293 cells mixed with Matrigel, by subcutaneous injection at rear flank. After inoculation, the xenografted tumors were allowed to grow for ~2-4 weeks till the tumor weight reached to around 100-250 mm³. Biodistribution studies were performed by administrating ¹⁷⁷Lu labeled complexes of representative compounds of the application at a dose of ~30-100 μCi per mouse (in 100 μL 0.9% NaCl, ~1.1-3.7 MBq, with specific activity 50-500 μCi/nmol) through tail vein injection.

At different time points after the administration, the mice treated in each group were euthanized. The tissues and organs of the animals were collected, excised, weighed, and analyzed in a PerkinElmer 2480 WIZARD2 γ counter, and the percent injected dose (% ID) and % ID/g of each organ or tissue were calculated.

Tables 7-10 below illustrate the biodistribution of the tested ¹⁷⁷Lu-labeled conjugates targeting Claudin 18.2 in tumor bearing mice at 1, 4, 24 and 96 hours after administration.

It was found that, as compared to the comparator DOTAGA-Lys(PEG3-Suc-CLDN18.2C(S))-Tyr-NH2 without the potency enhancing moiety of the present application, the conjugates targeting Claudin 18.2 (HO-C18-gGlu-OEG-OEG-OEG-Lys(DOTAGA)-Lys(PEG3-Suc-CLDN18.2C(S))-Tyr-NH2) had significantly higher tumor uptake, lower kidney uptake, and improved T/K at 24 h. Such unexpected improvements of the biodistribution offer the conjugates significantly more favorable profiles concerning therapeutic efficacy and therapeutic safety window.

TABLE 7
Biodistribution of ¹⁷⁷Lu-DOTAGA-
Lys(PEG3-Suc-CLDN18.2C(S))-Tyr-NH2
(ID %/g, Mean ± SD, n = 3)

| | 1 h | 4 h | 24 h |
|---|---|---|---|
| Pancreas | 0.46 ± 0.12 | 0.14 ± 0.10 | 0.07 ± 0.02 |
| Kidney | 237.99 ± 181.24 | 168.85 ± 119.20 | 288.06 ± 71.70 |
| Blood | 1.76 ± 0.68 | 0.15 ± 0.09 | 0.08 ± 0.03 |
| Heart | 0.98 ± 0.44 | 0.14 ± 0.12 | 0.21 ± 0.09 |
| Liver | 2.88 ± 1.00 | 3.57 ± 3.20 | 8.19 ± 4.21 |
| Spleen | 2.00+0.54 | 3.44 ± 3.09 | 3.12 ± 1.46 |
| Lung | 5.99 ± 3.26 | 3.52 ± 3.01 | 2.43 ± 2.41 |
| Stomach | 5.63 ± 4.71 | 0.41 ± 0.38 | 1.01 ± 1.29 |
| Intestinal | 0.71 ± 0.32 | 0.20 ± 0.12 | 0.08 ± 0.02 |
| Bone | 1.05 ± 0.19 | 0.44 ± 0.32 | 1.06 ± 0.47 |
| Muscle | 0.24 ± 0.19 | 0.02 ± 0.02 | 0.03 ± 0.03 |
| Tumor | 7.72 ± 1.04 | 1.81 ± 1.02 | 1.77 ± 1.34 |

TABLE 8
Biodistribution of ¹⁷⁷Lu-HO-C18-gGlu-OEG-OEG-OEG-Lys(DOTAGA)-
Lys(PEG3-Suc-CLDN18.2C(S))-Tyr-NH2 (ID %/g, Mean ± SD, n = 3)

| | 4 h | 24 h | 96 h |
|---|---|---|---|
| Pancreas | 1.14 ± 0.51 | 0.98 ± 0.37 | 0.30 ± 0.02 |
| Kidney | 63.96 ± 28.26 | 128.94 ± 54.52 | 31.74 ± 14.59 |
| Blood | 27.12 ± 3.53 | 5.93 ± 0.26 | 0.58 ± 0.08 |
| Heart | 4.72 ± 2.53 | 2.54 ± 0.30 | 0.63 ± 0.02 |
| Liver | 14.28 ± 8.74 | 10.84 ± 1.84 | 7.68 ± 4.16 |
| Spleen | 5.67 ± 3.68 | 8.55 ± 4.84 | 6.77 ± 0.34 |
| Lung | 3.39 ± 1.03 | 1.50 ± 0.39 | 0.90 ± 0.18 |
| Stomach | 5.65 ± 2.93 | 2.00 ± 0.70 | 0.63 ± 0.03 |
| Intestinal | 2.71 ± 1.04 | 0.79 ± 0.06 | 0.41 ± 0.10 |
| Bone | 2.32 ± 1.43 | 3.30 ± 0.44 | 1.55 ± 0.04 |
| Muscle | 0.99 ± 0.06 | 0.63 ± 0.18 | 0.23 ± 0.06 |
| Tumor | 6.04 ± 0.19 | 8.22 ± 4.54 | 3.84 ± 0.98 |

TABLE 9
Tumor to normal organ ratio of biodistribution of representative
CLDN18.2 targeting conjugates 24 hours after administration

| | T(% ID/g)/ | T(% ID/g)/ | T(% ID/g)/ |
|---|---|---|---|
| | K(% ID/g) | B(% ID/g) | M(% ID/g) |
| DOTAGA-Lys(PEG3-Suc- | 0.006 | 20.6 | 66.38 |
| CLDN18.2C(S))-Tyr-NH2 | | | |
| HO-C18-gGlu-OEG-OEG-OEG- | 0.1 | 1.4 | 13.06 |
| Lys(DOTAGA)-Lys(PEG3-Suc- | | | |
| CLDN18.2C(S))-Tyr-NH2 | | | |

TABLE 10
Tumor to normal organ ratio of biodistribution of representative
CLDN18.2 targeting conjugates 96 hours after administration

| | T(% ID/g)/ | T(% ID/g)/ | T(% ID/g)/ |
|---|---|---|---|
| | K(% ID/g) | B(% ID/g) | M(% ID/g) |
| HO-C18-gGlu-OEG-OEG-OEG- | 0.1 | 6.6 | 16.75 |
| Lys(DOTAGA)-Lys(PEG3-Suc- | | | |
| CLDN18.2C(S))-Tyr-NH2 | | | |

To evaluate the in vivo biodistribution of the sdAb conjugates targeting GCC, each mouse was inoculated with approximately 1×10⁷ human GCC expressing HEK-293 cells mixed with Matrigel, by subcutaneous injection at rear flank. After inoculation, the xenografted tumors were allowed to grow for −0.2-4 weeks till the tumor weight reached to around 100-250 mm³. Biodistribution studies were performed by administrating ¹⁷⁷Lu labeled complexes of representative compounds of the application at a dose of ~30-100 μCi per mouse (in 100 μL 0.9% NaCl, ~1.1-3.7 MBq, with specific activity 50-500 μCi/nmol) through tail vein injection.

At different time points after the administration, the mice treated in each group were euthanized. The tissues and organs of the animals were collected, excised, weighed, and analyzed in a PerkinElmer 2480 WIZARD2 γ counter, and the percent injected dose (% ID) and % ID/g of each organ or tissue were calculated.

Tables 11-20 below illustrate the biodistribution of the tested ¹⁷⁷Lu-labeled conjugates targeting GCC in tumor bearing mice at 24 hours after administration.

It was found that, as compared to the comparator conjugate ¹⁷⁷Lu-DOTAGA-Lys(PEG3-Suc-GCCC(S))-Tyr-NH2, the conjugates including ¹⁷⁷Lu-HO-C18-gGlu-PEG8-PEG8-PEG8-PEG8-Lys(DOTAGA)-Lys(PEG3-Suc-GCCC(S))-Tyr-NH2, ¹⁷⁷Lu-[HO-C18-gGlu-PEG8-PEG8-PEG8-PEG8]2-Lys-Lys(DOTAGA)-Lys(PEG3-Suc-GCCC(S))-Tyr-NH2, ¹⁷⁷Lu-HO-C18-gGlu-PEG8-Nal-PEG8-Nal-PEG8-Lys(ESL1-DOTAGA)-Lys(PEG3-Suc-GCCC(S))-Tyr-NH2, ¹⁷⁷Lu-[HO-C18-gGlu-PEG8-PEG8-PEG8-PEG8]2-Lys-Lys(ESL1-DOTAGA)-Lys(PEG3-Suc-GCCC(S))-Tyr-NH2, ¹⁷⁷Lu-[HO-C18-gGlu-PEG8-PEG8]2-Lys-Lys(ESL1-DOTAGA)-Lys(PEG3-Suc-GCCC(S))-Tyr-NH2, ¹⁷⁷Lu-[HO-C18-gGlu-gGlu-gGlu-OEG-OEG]2-Lys-ESL1-Lys(DOTAGA)-Lys(PEG3-Suc-GCCC(S))-Tyr-NH2, and ¹⁷⁷Lu-[HO-C18-gGlu-PEG8-PEG8]2-Lys-ESL1-Lys(DOTAGA)-Lys(PEG3-Suc-GCCC(S))-Tyr-NH2 all displayed significantly higher tumor uptake, lower kidney uptake, and improved T/K at 24 h.

In addition, the conjugates with cleavable linkers as described herein also demonstrated superior tumor uptake to normal organ uptake ratios, including tumor to blood and tumor to muscle, over the ones with non-cleavable linkers, for example:
(1) cleavable ¹⁷⁷Lu-HO-C18-gGlu-PEG8-Nal-PEG8-Nal-PEG8-Lys(ESL1-DOTAGA)-Lys(PEG3-Suc-GCCC(S))-Tyr-NH2 vs non-cleavable ¹⁷⁷Lu-HO-C18-gGlu-PEG8-PEG8-PEG8-PEG8-Lys(DOTAGA)-Lys(PEG3-Suc-GCCC(S))-Tyr-NH2, and
(2) cleavable ¹⁷⁷Lu-[HO-C18-gGlu-PEG8-PEG8-PEG8-PEG8]2-Lys-Lys(ESL1-DOTAGA)-Lys(PEG3-Suc-GCCC(S))-Tyr-NH2, ¹⁷⁷Lu-[HO-C18-gGlu-PEG8-PEG8]2-Lys-Lys(ESL1-DOTAGA)-Lys(PEG3-Suc-GCCC(S))-Tyr-NH2, ¹⁷⁷Lu-[HO-C18-gGlu-gGlu-gGlu-OEG-OEG]2-Lys-ESL1-Lys(DOTAGA)-Lys(PEG3-Suc-GCCC(S))-Tyr-NH2, and ¹⁷⁷Lu-[HO-C18-gGlu-PEG8-PEG8]2-Lys-ESL1-Lys(DOTAGA)-Lys(PEG3-Suc-GCCC(S))-Tyr-NH2 vs non-cleavable ¹⁷⁷Lu-[HO-C18-gGlu-PEG8-PEG8-PEG8-PEG8]2-Lys-Lys(DOTAGA)-Lys(PEG3-Suc-GCCC(S))-Tyr-NH2.

Such unexpected further improvements of the biodistribution brought by the cleavable linkers offer the conjugates significantly more favorable profiles concerning therapeutic efficacy and therapeutic safety window.

TABLE 11
Biodistribution of ¹⁷⁷Lu-DOTAGA-Lys(PEG3-Suc-GCCC(S))-Tyr-NH2
(ID %/g, Mean ± SD, n = 3)

| | 24 h |
|---|---|
| Pancreas | 0.35 ± 0.24 |
| Kidney | 69.89 ± 3.59 |
| Blood | 0.08 ± 0.01 |
| Heart | 0.15 ± 0.04 |
| Liver | 10.72 ± 9.90 |
| Spleen | 3.45 ± 3.49 |
| Lung | 0.74 ± 0.71 |
| Stomach | 0.23 ± 0.10 |
| Intestinal | 0.43 ± 0.12 |
| Bone | 0.71 ± 0.10 |
| Muscle | 0.06 ± 0.01 |
| Tumor | 3.32 ± 0.90 |

TABLE 12
Biodistribution of ¹⁷⁷Lu-HO-C18-gGlu-PEG8-PEG8-
PEG8-PEG8-Lys(DOTAGA)-Lys(PEG3-Suc-GCCC(S))-
Tyr-NH2 (ID %/g, Mean ± SD, n = 3)

| | 24 h | 72 h |
|---|---|---|
| Pancreas | 2.19 ± 1.33 | 0.57 ± 0.06 |
| Kidney | 33.12 ± 6.69 | 17.83 ± 1.82 |
| Blood | 4.12 ± 0.72 | 0.83 ± 0.14 |
| Heart | 1.94 ± 0.35 | 0.88 ± 0.06 |
| Liver | 47.49 ± 2.90 | 36.01 ± 9.80 |
| Spleen | 9.07 ± 0.23 | 5.05 ± 1.26 |
| Lung | 3.04 ± 0.57 | 1.83 ± 0.35 |
| Stomach | 1.64 ± 0.17 | 0.59 ± 0.45 |
| Intestinal | 2.17 ± 0.46 | 1.16 ± 0.57 |
| Bone | 3.51 ± 0.75 | 3.37 ± 0.05 |
| Muscle | 0.73 ± 0.18 | 0.35 ± 0.03 |
| Tumor | 9.72 ± 2.40 | 6.28 ± 1.65 |

TABLE 13
Biodistribution of ¹⁷⁷Lu-[HO-C18-gGlu-PEG8-PEG8-PEG8-
PEG8]2-Lys-Lys(DOTAGA)-Lys(PEG3-Suc-GCCC(S))-
Tyr-NH2 (ID %/g, Mean ± SD, n = 3)

| | 24 h | 72 h |
|---|---|---|
| Pancreas | 3.18 ± 1.04 | 1.95 ± 0.56 |
| Kidney | 8.87 ± 1.63 | 5.62 ± 1.00 |
| Blood | 24.04 ± 3.23 | 10.69 ± 2.88 |
| Heart | 7.96 ± 1.41 | 4.70 ± 0.91 |
| Liver | 21.10 ± 6.78 | 18.82 ± 7.91 |
| Spleen | 13.64 ± 2.21 | 12.15 ± 2.93 |
| Lung | 13.13 ± 3.13 | 6.38 ± 1.04 |
| Stomach | 7.18 ± 2.52 | 1.89 ± 0.32 |
| Intestinal | 7.34 ± 1.59 | 2.84 ± 0.20 |
| Bone | 4.88 ± 0.51 | 3.20 ± 0.50 |
| Muscle | 2.13 ± 0.27 | 1.27 ± 0.23 |
| Tumor | 11.51 ± 1.11 | 15.28 ± 7.47 |

TABLE 14
Biodistribution of ¹⁷⁷Lu-HO-C18-gGlu-PEG8-Nal-PEG8-
Nal-PEG8-Lys(ESL1-DOTAGA)-Lys(PEG3-Suc-
GCCC(S))-Tyr-NH2 (ID %/g, Mean ± SD, n = 3)

| | 2 4h | 72 h |
|---|---|---|
| Pancreas | 1.03 ± 0.18 | 0.84 ± 0.07 |
| Kidney | 161.35 ± 17.82 | 129.87 ± 13.20 |
| Blood | 3.27 ± 0.53 | 0.92 ± 0.12 |
| Heart | 3.44 ± 2.30 | 1.01 ± 0.09 |
| Liver | 12.08 ± 1.69 | 14.33 ± 0.70 |
| Spleen | 5.56 ± 0.71 | 7.52 ± 1.49 |
| Lung | 2.60 ± 0.31 | 1.40 ± 0.21 |
| Stomach | 1.73 ± 0.72 | 0.64 ± 0.11 |
| Intestinal | 1.56 ± 0.17 | 1.28 ± 0.10 |
| Bone | 2.18 ± 0.75 | 2.08 ± 0.03 |
| Muscle | 0.73 ± 0.18 | 0.43 ± 0.06 |
| Tumor | 17.41 ± 3.92 | 10.25 ± 4.29 |

TABLE 15
Biodistribution of ¹⁷⁷Lu-[HO-C18-gGlu-PEG8-PEG8-PEG8-
PEG8]2-Lys-Lys(ESL1-DOTAGA)-Lys(PEG3-Suc-GCCC(S))-
Tyr-NH2 (ID %/g, Mean ± SD, n = 3)

| | 24 h | 72 h |
|---|---|---|
| Pancreas | 2.45 ± 0.26 | 1.73 ± 0.29 |
| Kidney | 7.56 ± 0.74 | 5.13 ± 0.38 |
| Blood | 24.16 ± 2.48 | 8.06 ± 0.55 |
| Heart | 8.47 ± 1.74 | 4.36 ± 0.33 |
| Liver | 9.97 ± 0.95 | 7.67 ± 0.40 |
| Spleen | 8.02 ± 1.80 | 6.71 ± 1.05 |
| Lung | 9.40 ± 0.70 | 5.34 ± 0.29 |
| Stomach | 2.65 ± 0.22 | 1.18 ± 0.15 |
| Intestinal | 3.79 ± 0.20 | 2.19 ± 0.33 |
| Bone | 2.97 ± 0.06 | 2.12 ± 0.64 |
| Muscle | 2.11 ± 0.31 | 1.24 ± 0.06 |
| Tumor | 26.53 ± 6.40 | 18.32 ± 3.53 |

TABLE 16
Biodistribution of ¹⁷⁷Lu-[HO-C18-gGlu-PEG8-PEG8]2-
Lys-Lys(ESL1-DOTAGA)-Lys(PEG3-Suc-GCCC(S))-
Tyr-NH2 (ID %/g, Mean ± SD, n = 3)

| | 24 h | 72 h |
|---|---|---|
| Pancreas | 1.38 ± 0.26 | 0.86 ± 0.13 |
| Kidney | 4.65 ± 0.84 | 3.34 ± 0.14 |
| Blood | 9.97 ± 0.41 | 3.54 ± 0.79 |
| Heart | 3.92 ± 0.66 | 1.40 ± 1.03 |
| Liver | 48.01 ± 2.49 | 31.76 ± 3.93 |
| Spleen | 23.93 ± 0.61 | 18.47 ± 2.14 |
| Lung | 6.27 ± 0.71 | 3.09 ± 0.18 |
| Stomach | 1.33 ± 0.21 | 0.82 ± 0.26 |
| Intestinal | 1.76 ± 0.25 | 1.15 ± 0.19 |
| Bone | 2.72 ± 0.47 | 2.49 ± 0.21 |
| Muscle | 0.97 ± 0.12 | 0.71 ± 0.14 |
| Tumor | 11.53 ± 0.83 | 10.63 ± 1.94 |

TABLE 17
Biodistribution of ¹⁷⁷Lu-[HO-C18-gGlu-gGlu-gGlu-OEG-OEG]2-
Lys-ESL1-Lys(DOTAGA)-Lys(PEG3-Suc-GCCC(S))-
Tyr-NH2 (ID %/g, Mean ± SD, n = 3)

| | 24 h | 72 h |
|---|---|---|
| Pancreas | 1.29 ± 0.08 | 0.79 ± 0.03 |
| Kidney | 7.68 ± 0.38 | 13.68 ± 12.09 |
| Blood | 7.73 ± 0.40 | 2.54 ± 0.17 |
| Heart | 2.88 ± 0.21 | 1.62 ± 0.18 |
| Liver | 71.18 ± 1.34 | 52.02 ± 3.87 |
| Spleen | 32.43 ± 7.61 | 29.51 ± 5.44 |
| Lung | 6.14 ± 1.49 | 2.46 ± 0.38 |
| Stomach | 1.31 ± 0.03 | 0.68 ± 0.12 |
| Intestinal | 1.97 ± 0.10 | 0.99 ± 0.09 |
| Bone | 2.63 ± 0.22 | 1.70 ± 0.30 |
| Muscle | 0.98 ± 0.13 | 0.55 ± 0.19 |
| Tumor | 6.88 ± 1.41 | 5.99 ± 0.86 |

TABLE 18
Biodistribution of ¹⁷⁷Lu-[HO-C18-gGlu-PEG8-PEG8]2-
Lys-ESL1-Lys(DOTAGA)-Lys(PEG3-Suc-GCCC(S))-
Tyr-NH2 (ID %/g, Mean ± SD, n = 3)

| | 24 h | 72 h |
|---|---|---|
| Pancreas | 3.01 ± 0.40 | 2.14 ± 0.47 |
| Kidney | 19.26 ± 0.98 | 15.57 ± 2.24 |
| Blood | 25.36 ± 3.32 | 7.98 ± 1.44 |
| Heart | 8.39 ± 1.63 | 4.57 ± 0.28 |
| Liver | 11.69 ± 0.82 | 9.25 ± 0.81 |
| Spleen | 8.28 ± 1.31 | 8.46 ± 1.50 |
| Lung | 12.73 ± 1.08 | 5.60 ± 1.71 |
| Stomach | 3.22 ± 0.39 | 1.64 ± 0.44 |
| Intestinal | 4.28 ± 0.47 | 2.18 ± 0.05 |
| Bone | 4.31 ± 0.28 | 2.76 ± 0.49 |
| Muscle | 2.24 ± 0.28 | 1.37 ± 0.18 |
| Tumor | 25.01 ± 3.83 | 25.01 ± 4.46 |

TABLE 19
Tumor to normal organ ratio of biodistribution of representative GCC
targeting conjugates 24 hours after administration

| | | | | T(% ID)/ |
|---|---|---|---|---|
| | T(% ID/g)/ | T(% ID/g)/ | T(% ID/g)/ | normalized |
| | K(% ID/g) | B(% ID/g) | M(% ID/g) | B (% ID)* |
| DOTAGA-Lys(PEG3-Suc-GCCC(S))-Tyr-NH2 | 0.05 | 40.2 | 52.42 | 17.65 |
| HO-C18-gGlu-PEG8-PEG8-PEG8-PEG8- | 0.3 | 2.4 | 13.27 | 1.51 |
| Lys(DOTAGA)-Lys(PEG3-Suc-GCCC(S))-Tyr- | | | | |
| NH2 | | | | |
| [HO-C18-gGlu-PEG8-PEG8-PEG8-PEG8]2-Lys- | 1.3 | 0.5 | 5.41 | 0.26 |
| Lys(DOTAGA)-Lys(PEG3-Suc-GCCC(S))-Tyr- | | | | |
| NH2 | | | | |
| HO-C18-gGlu-PEG8-Nal-PEG8-Nal-PEG8- | 0.1 | 5.3 | 23.74 | 2.08 |
| Lys(ESL1-DOTAGA)-Lys(PEG3-Suc- | | | | |
| GCCC(S))-Tyr-NH2 | | | | |
| [HO-C18-gGlu-PEG8-PEG8-PEG8-PEG8]2-Lys- | 3.5 | 1.1 | 12.55 | 0.36 |
| Lys(ESL1-DOTAGA)-Lys(PEG3-Suc- | | | | |
| GCCC(S))-Tyr-NH2 | | | | |
| [HO-C18-gGlu-PEG8-PEG8]2-Lys-Lys(ESL1- | 2.5 | 1.2 | 11.89 | 0.38 |
| DOTAGA)-Lys(PEG3-Suc-GCCC(S))Tyr-NH2 | | | | |
| [HO-C18-gGlu-Glu-gGlu-OEG-OEG]2-Lys- | 0.9 | 0.9 | 7.02 | 0.14 |
| ESL1-Lys(DOTAGA)-Lys(PEG3-Suc- | | | | |
| GCCC(S))-Tyr-NH2 | | | | |
| HO-C18-gGlu-PEG8-PEG8]2-Lys-ESL1- | 1.3 | 1.0 | 11.15 | 0.16 |
| Lys(DOTAGA)-Lys(PEG3-Suc-GCCC(S))-Tyr- | | | | |
| NH2 | | | | |

*Note:
T(% ID)/normalized B (% ID) represents the ratio of average T % ID (T% ID/g * tumor weight) to average B % ID [B% ID/g * blood weight (body weight *7.5%)] normalized by each gram of blood in mice, which is to indicate the ratio of uptake in tumor organ to blood.

TABLE 20
Tumor to normal organ ratio of biodistribution of representative GCC
targeting conjugates 72 hours after administration

| | | | | T(% ID)/ |
|---|---|---|---|---|
| | T(% ID/g)/ | T(% ID/g)/ | T(% ID/g)/ | normalized |
| | K(% ID/g) | B(% ID/g) | M(% ID/g) | B (% ID)* |
| HO-C18-gGlu-PEG8-PEG8-PEG8-PEG8- | 0.4 | 7.5 | 17.87 | 4.86 |
| Lys(DOTAGA)-Lys(PEG3-Suc-GCCC(S))-Tyr- | | | | |
| NH2 | | | | |
| [HO-C18-gGlu-PEG8-PEG8-PEG8-PEG8]2-Lys- | 2.8 | 1.5 | 12.00 | 0.82 |
| Lys(DOTAGA)-Lys(PEG3-Suc-GCCC(S))-Tyr- | | | | |
| NH2 | | | | |
| HO-C18-gGlu-PEG8-Nal-PEG8-Nal-PEG8- | 0.1 | 11.6 | 24.02 | 4.31 |
| Lys(ESL1-DOTAGA)-Lys(PEG3-Suc- | | | | |
| GCCC(S))-Tyr-NH2 | | | | |
| [HO-C18-gGlu-PEG8-PEG8-PEG8-PEG8]2-Lys- | 3.6 | 2.3 | 14.78 | 1.20 |
| Lys(ESL1-DOTAGA)-Lys(PEG3-Suc- | | | | |
| GCCC(S))-Tyr-NH2 | | | | |
| [HO-C18-gGlu-PEG8-PEG8]2-Lys-Lys(ESL1- | 3.2 | 3.0 | 15.05 | 1.32 |
| DOTAGA)-Lys(PEG3-Suc-GCCC(S))-Tyr-NH2 | | | | |
| [HO-C18-gGlu-gGlu-gGlu-OEG-OEG]2-Lys- | 0.7 | 2.4 | 10.88 | 0.60 |
| ESL1-Lys(DOTAGA)-Lys(PEG3-Suc- | | | | |
| GCCC(S))-Tyr-NH2 | | | | |
| [HO-C18-gGlu-PEG8-PEG8]2-Lys-ESL1- | 1.6 | 3.2 | 18.21 | 0.67 |
| Lys(DOTAGA)-Lys(PEG3-Suc-GCCC(S))-Tyr- | | | | |
| NH2 | | | | |

*Note:
T(% ID)/normalized B (% ID) represents the ratio of average T % ID (T% ID/g * tumor weight) to average B % ID [B% ID/g * blood weight (body weight *7.5%)] normalized by each gram of blood in mice, which is to indicate the ratio of uptake in tumor organ to blood.

To evaluate the in vivo biodistribution of the sdAb conjugates targeting FAP, each mouse was inoculated with approximately 1×10⁷ human FAP expressing 293T cells mixed with Matrigel, by subcutaneous injection at rear flank. After inoculation, the xenografted tumors were allowed to grow for ~2-4 weeks till the tumor weight reached to around 100-250 mm³.

Biodistribution studies were performed by administrating ¹⁷⁷Lu labeled complexes of representative compounds of the application at a dose of ~30-100 μCi per mouse (in 100 μL 0.9% NaCl, ~1.1-3.7 MBq, with specific activity 50-500 μCi/nmol) through tail vein injection.

At different time points after the administration, the mice treated in each group were euthanized. The tissues and organs of the animals were collected, excised, weighed, and analyzed in a PerkinElmer 2480 WIZARD2 7 counter, and the percent injected dose (% ID) and % ID/g of each organ or tissue were calculated.

Tables 21-32 below illustrate the biodistribution of the tested ¹⁷⁷Lu labeled conjugates targeting FAP in tumor bearing mice at 24 and 72 hour after administration.

It was found that, as compared to the comparator conjugate ¹⁷⁷Lu-DOTAGA-Lys(PEG3-Suc-FAPC(S))-Tyr-NH2, the conjugates including ¹⁷⁷Lu-[HO-C18-gGlu-PEG8-PEG8]2-Lys-Lys(PEG8-ESL1-PEG8-DOTAGA)-Lys(PEG3-Suc-FAPC(S))-Tyr-NH2, ¹⁷⁷Lu-HO-C20-gGlu-PEG8-PEG8-PEG8-PEG8-Lys(DOTAGA)-Lys(PEG3-Suc-FAPC(S))-Tyr-NH2, ¹⁷⁷Lu-HO-C20-gGlu-PEG8-PEG8-PEG8-PEG8-Lys(ESL1-DOTAGA)-Lys(PEG3-Suc-FAPC(S))-Tyr-NH2, ¹⁷⁷Lu-HO-C20-gGlu-PEG8-PEG8-PEG8-PEG8-Lys(PEG8-ESL1-PEG8-DOTAGA)-Lys(PEG3-Suc-FAPC(S))-Tyr-NH2, ¹⁷⁷Lu-HO-C20-gGlu-PEG8-PEG8-PEG8-PEG8-Lys(PEG8-ESL1-OEG-DOTAGA)-Lys(PEG3-Suc-FAPC(S))-Tyr-NH2, ¹⁷⁷Lu-HO-C20-gGlu-PEG8-PEG8-PEG8-PEG8-Lys(PEG8-ESL1-PEG5-DOTAGA)-Lys(PEG3-Suc-FAPC(S))-Tyr-NH2, ¹⁷⁷Lu-HO-C20-gGlu-PEG8-PEG8-PEG8-PEG8-Lys(PEG8-ESL1-PEG7-DOTAGA)-Lys(PEG3-Suc-FAPC(S))-Tyr-NH2, ¹⁷⁷Lu-[HO-C18-gGlu-PEG8-PEG8-PEG8-PEG8]2-Lys-Lys(PEG8-ESL1-PEG8-DOTAGA)-Lys(PEG3-Suc-FAPC(S))-Tyr-NH2, and ¹⁷⁷Lu-[HO-C18-gGlu-PEG8-PEG8-PEG8-PEG8]2-Lys-Lys(PEG8-OEG-PEG8-DOTAGA)-Lys(PEG3-Suc-FAPC(S))-Tyr-NH2 all displayed significantly higher tumor uptake, lower kidney uptake, and improved T/K at 24 h.

In addition, the conjugates with cleavable linkers as described herein also demonstrated superior tumor uptake to normal organ uptake ratios, including tumor to blood and tumor to muscle, over the ones modified with non-cleavable linkers, for example:
(1) cleavable ¹⁷⁷Lu-HO-C20-gGlu-PEG8-PEG8-PEG8-PEG8-Lys(ESL1-DOTAGA)-Lys(PEG3-Suc-FAPC(S))-Tyr-NH2, ¹⁷⁷Lu-HO-C20-gGlu-PEG8-PEG8-PEG8-PEG8-Lys(PEG8-ESL1-PEG8-DOTAGA)-Lys(PEG3-Suc-FAPC(S))-Tyr-NH2, ¹⁷⁷Lu-HO-C20-gGlu-PEG8-PEG8-PEG8-PEG8-Lys(PEG8-ESL1-OEG-DOTAGA)-Lys(PEG3-Suc-FAPC(S))-Tyr-NH2, ¹⁷⁷Lu-HO-C20-gGlu-PEG8-PEG8-PEG8-PEG8-Lys(PEG8-ESL1-PEG5-DOTAGA)-Lys(PEG3-Suc-FAPC(S))-Tyr-NH2, and ¹⁷⁷Lu-HO-C20-gGlu-PEG8-PEG8-PEG8-PEG8-Lys(PEG8-ESL1-PEG7-DOTAGA)-Lys(PEG3-Suc-FAPC(S))-Tyr-NH2 vs non cleavable ¹⁷⁷Lu-HO-C20-gGlu-PEG8-PEG8-PEG8-PEG8-Lys(DOTAGA)-Lys(PEG3-Suc-FAPC(S))-Tyr-NH2; and
(2) cleavable ¹⁷⁷Lu-[HO-C18-gGlu-PEG8-PEG8]2-Lys-Lys(PEG8-ESL1-PEG8-DOTAGA)-Lys(PEG3-Suc-Z)-Tyr-NH2 and ¹⁷⁷Lu-[HO-C18-gGlu-PEG8-PEG8-PEG8-PEG8]2-Lys-Lys(PEG8-ESL1-PEG8-DOTAGA)-Lys(PEG3-Suc-FAPC(S))-Tyr-NH2 vs non-cleavable ¹⁷⁷Lu-[HO-C18-gGlu-PEG8-PEG8-PEG8-PEG8]2-Lys-Lys(PEG8-OEG-PEG8-DOTAGA)-Lys(PEG3-Suc-FAPC(S))-Tyr-NH2.

Such unexpected further improvements of the biodistribution brought by the cleavable linkers offer the conjugates significantly more favorable profiles concerning therapeutic efficacy and therapeutic safety window.

TABLE 21
Biodistribution of ¹⁷⁷Lu-DOTAGA-Lys(PEG3-Suc-
FAPC(S))-Tyr-NH2 (ID %/g, Mean ± SD, n = 3)

| | 4 h | 24 h |
|---|---|---|
| Pancreas | 0.12 ± 0.01 | 0.10 ± 0.02 |
| Kidney | 351.02 ± 47.20 | 222.93 ± 59.88 |
| Blood | 0.15 ± 0.04 | 0.08 ± 0.01 |
| Heart | 0.88 ± 1.26 | 0.17 ± 0.01 |
| Liver | 4.17 ± 0.91 | 3.01 ± 0.35 |
| Spleen | 1.78 ± 0.34 | 1.37 ± 0.31 |
| Lung | 0.66 ± 0.18 | 0.32 ± 0.16 |
| Stomach | 0.29 ± 0.04 | 0.16 ± 0.03 |
| Intestinal | 0.32 ± 0.06 | 0.22 ± 0.07 |
| Bone | 0.42 ± 0.04 | 0.34 ± 0.03 |
| Muscle | 0.16 ± 0.07 | 0.09 ± 0.05 |
| Tumor | 5.83 ± 0.91 | 2.60 ± 0.93 |

TABLE 22
Biodistribution of ¹⁷⁷Lu-[HO-C18-gGlu-PEG8-PEG8]2-Lys-
Lys(PEG8-ESL1-PEG8-DOTAGA)-Lys(PEG3-Suc-FAPC(S))-
Tyr-NH2 (ID %/g, Mean ± SD, n = 3)

| | 24 h |
|---|---|
| Pancreas | 1.01 ± 0.06 |
| Kidney | 5.13 ± 1.12 |
| Blood | 5.44 ± 0.32 |
| Heart | 2.08 ± 0.25 |
| Liver | 20.72 ± 1.04 |
| Spleen | 4.43 ± 0.68 |
| Lung | 3.24 ± 0.51 |
| Stomach | 1.11 ± 0.11 |
| Intestinal | 1.57 ± 0.14 |
| Bone | 2.32 ± 0.28 |
| Muscle | 0.90 ± 0.16 |
| Tumor | 8.07 ± 1.90 |

TABLE 23
Biodistribution of ¹⁷⁷Lu-HO-C20-gGlu-PEG8-PEG8-
PEG8-PEG8-Lys(DOTAGA)-Lys(PEG3-Suc-FAPC(S))-
Tyr-NH2 (ID %/g, Mean ± SD, n = 3)

| | 24 h | 72 h |
|---|---|---|
| Pancreas | 4.19 ± 0.74 | 2.13 ± 0.37 |
| Kidney | 32.91 ± 1.64 | 19.90 ± 2.28 |
| Blood | 20.63 ± 2.17 | 6.25 ± 1.24 |
| Heart | 7.15 ± 0.76 | 3.62 ± 0.29 |
| Liver | 13.20 ± 0.63 | 9.59 ± 1.05 |
| Spleen | 11.37 ± 0.84 | 8.43 ± 0.71 |
| Lung | 13.36 ± 2.18 | 5.94 ± 0.46 |
| Stomach | 4.48 ± 0.98 | 1.75 ± 0.16 |
| Intestinal | 6.40 ± 0.92 | 3.96 ± 0.65 |
| Bone | 4.63 ± 0.69 | 2.66 ± 0.30 |
| Muscle | 2.26 ± 0.40 | 1.87 ± 0.01 |
| Tumor | 49.67 ± 5.54 | 45.61 ± 1.09 |

TABLE 24
Biodistribution of ¹⁷⁷Lu-HO-C20-gGlu-PEG8-PEG8-PEG8-
PEG8-Lys(ESL1-DOTAGA)-Lys(PEG3-Suc-FAPC(S))-
Tyr-NH2 (ID %/g, Mean ± SD, n = 3)

| | 24 h | 72 h |
|---|---|---|
| Pancreas | 3.37 ± 0.32 | 1.46 ± 0.16 |
| Kidney | 29.46 ± 1.47 | 12.46 ± 1.29 |
| Blood | 17.88 ± 1.64 | 3.66 ± 0.54 |
| Heart | 6.79 ± 0.22 | 2.44 ± 0.29 |
| Liver | 13.28 ± 1.22 | 6.81 ± 0.38 |
| Spleen | 9.86 ± 2.23 | 5.98 ± 1.63 |
| Lung | 10.64 ± 0.94 | 4.26 ± 1.02 |
| Stomach | 4.10 ± 0.49 | 1.07 ± 0.06 |
| Intestinal | 5.43 ± 0.64 | 2.37 ± 0.26 |
| Bone | 4.27 ± 0.44 | 1.77 ± 0.31 |
| Muscle | 2.37 ± 0.31 | 0.99 ± 0.20 |
| Tumor | 41.74 ± 1.08 | 36.40 ± 3.46 |

TABLE 25
Biodistribution of ¹⁷⁷Lu-HO-C20-gGlu-
PEG8-PEG8-PEG8-PEG8-Lys
(PEG8-ESL1-PEG8-DOTAGA)-Lys
(PEG3-Suc-FAPC(S))-Tyr-NH2
(ID %/g, Mean ± SD, n ± 3)

| | 24 h | 72 h |
|---|---|---|
| Pancreas | 0.32 ± 0.06 | 0.09 ± 0.01 |
| Kidney | 6.05 ± 0.42 | 1.52 ± 0.59 |
| Blood | 0.96 ± 0.04 | 0.11 ± 0.01 |
| Heart | 0.46 ± 0.06 | 0.09 ± 0.01 |
| Liver | 8.75 ± 1.61 | 4.16 ± 0.53 |
| Spleen | 5.91 ± 3.34 | 2.53 ± 0.60 |
| Lung | 0.86 ± 0.08 | 0.18 ± 0.03 |
| Stomach | 0.42 ± 0.10 | 0.09 ± 0.02 |
| Intestinal | 0.49 ± 0.05 | 0.13 ± 0.02 |
| Bone | 0.58 ± 0.06 | 0.37 ± 0.02 |
| Muscle | 0.22 ± 0.07 | 0.04 ± 0.01 |
| Tumor | 5.09 ± 0.50 | 1.55 ± 0.18 |

TABLE 26
Biodistribution of ¹⁷⁷Lu-HO-C20-gGlu-
PEG8-PEG8-PEG8-PEG8-Lys
(PEG8-ESL1-OEG-DOTAGA)-Lys
(PEG3-Suc-FAPC(S))-Tyr-NH2
(ID %/g, Mean ± SD, n ± 3)

| | 24 h | 72 h |
|---|---|---|
| Pancreas | 2.50 ± 0.13 | 1.25 ± 0.15 |
| Kidney | 25.15 ± 2.38 | 11.96 ± 1.05 |
| Blood | 15.61 ± 1.79 | 2.90 ± 0.13 |
| Heart | 5.69 ± 0.65 | 1.91 ± 0.22 |
| Liver | 12.41 ± 1.22 | 7.25 ± 0.55 |
| Spleen | 6.19 ± 0.88 | 4.35 ± 0.74 |
| Lung | 8.98 ± 0.92 | 2.81 ± 0.22 |
| Stomach | 2.80 ± 0.46 | 0.78 ± 0.09 |
| Intestinal | 4.90 ± 0.52 | 1.71 ± 0.27 |
| Bone | 3.12 ± 0.15 | 0.96 ± 0.10 |
| Muscle | 1.51 ± 0.22 | 0.48 ± 0.04 |
| Tumor | 43.57 ± 7.92 | 20.20 ± 2.22 |

TABLE 27
Biodistribution of ¹⁷⁷Lu-HO-C20-gGlu-
PEG8-PEG8-PEG8-PEG8-Lys
(PEG8-ESL1-PEG5-DOTAGA)-Lys
(PEG3-Suc-FAPC(S))-Tyr-NH2
(ID %/g, Mean ± SD, n ± 3)

| | 24 h | 72 h |
|---|---|---|
| Pancreas | 0.76 ± 0.15 | 0.09 ± 0.15 |
| Kidney | 9.63 ± 0.61 | 2.53 ± 0.45 |
| Blood | 3.70 ± 0.24 | 0.34 ± 0.07 |
| Heart | 1.50 ± 0.18 | 0.08 ± 0.07 |
| Liver | 5.16 ± 0.73 | 2.46 ± 0.23 |
| Spleen | 2.89 ± 0.46 | 0.94 ± 0.06 |
| Lung | 2.26 ± 0.10 | 0.35 ± 0.07 |
| Stomach | 0.74 ± 0.10 | 0.00 ± 0.00 |
| Intestinal | 1.49 ± 0.20 | 0.40 ± 0.07 |
| Bone | 0.85 ± 0.03 | 0.08 ± 0.04 |
| Muscle | 0.43 ± 0.06 | 0.00 ± 0.00 |
| Tumor | 8.76 ± 0.94 | 3.90 ± 2.68 |

TABLE 28
Biodistribution of ¹⁷⁷Lu-HO-C20-gGlu-
PEG8-PEG8-PEG8-PEG8-Lys
(PEG8-ESL1-PEG7-DOTAGA)-Lys
(PEG3-Suc-FAPC(S))-Tyr-NH2
(ID %/g, Mean ± SD, n ± 3)

| | 24 h | 72 h |
|---|---|---|
| Pancreas | 0.36 ± 0.06 | 0.01 ± 0.02 |
| Kidney | 7.32 ± 0.14 | 2.12 ± 0.66 |
| Blood | 1.51 ± 0.03 | 0.22 ± 0.02 |
| Heart | 0.68 ± 0.16 | 0.10 ± 0.13 |
| Liver | 5.13 ± 0.20 | 1.62 ± 0.19 |
| Spleen | 2.08 ± 0.50 | 0.66 ± 0.16 |
| Lung | 0.91 ± 0.08 | 0.15 ± 0.02 |
| Stomach | 0.27 ± 0.05 | 0.01 ± 0.02 |
| Intestinal | 0.71 ± 0.03 | 0.15 ± 0.03 |
| Bone | 0.46 ± 0.10 | 0.00 ± 0.00 |
| Muscle | 0.17 ± 0.01 | 0.00 ± 0.00 |
| Tumor | 4.77 ± 1.16 | 2.02 ± 0.51 |

TABLE 29
Biodistribution of ¹⁷⁷Lu-[HO-C18-gGlu-
PEG8-PEG8-PEG8-PEG8]2-Lys-Lys
(PEG8-ESL1-PEG8-DOTAGA)-Lys
(PEG3-Suc-FAPC(S))-Tyr-NH2
(ID %/g, Mean ± SD, n ± 3)

| | 24 h | 72 h |
|---|---|---|
| Pancreas | 0.96 ± 0.26 | 0.11 ± 0.04 |
| Kidney | 3.22 ± 0.64 | 0.95 ± 0.16 |
| Blood | 5.38 ± 0.91 | 0.50 ± 0.06 |
| Heart | 1.65 ± 0.25 | 0.18 ± 0.08 |
| Liver | 6.03 ± 1.53 | 2.88 ± 1.10 |
| Spleen | 2.70 ± 0.38 | 0.88 ± 0.33 |
| Lung | 2.89 ± 0.38 | 0.48 ± 0.12 |
| Stomach | 0.87 ± 0.17 | 0.07 ± 0.03 |
| Intestinal | 1.64 ± 0.25 | 0.47 ± 0.18 |
| Bone | 1.24 ± 0.37 | 0.55 ± 0.08 |
| Muscle | 0.60 ± 0.22 | 0.00 ± 0.00 |
| Tumor | 7.62 ± 1.71 | 3.26 ± 0.89 |

TABLE 30
Biodistribution of ¹⁷⁷Lu-[HO-C18-gGlu-
PEG8-PEG8-PEG8-PEG8]2-Lys-Lys
(PEG8-OEG-PEG8-DOTAGA)-Lys
(PEG3-Suc-FAPC(S))-Tyr-NH2
(ID %/g, Mean ± SD, n ± 3)

| | 24 h | 72 h |
|---|---|---|
| Pancreas | 3.22 ± 0.40 | 2.65 ± 0.54 |
| Kidney | 7.86 ± 0.59 | 5.42 ± 0.44 |
| Blood | 24.98 ± 1.86 | 9.03 ± 0.42 |
| Heart | 7.76 ± 1.06 | 4.03 ± 0.28 |
| Liver | 14.72 ± 1.63 | 13.30 ± 3.80 |
| Spleen | 8.19 ± 1.16 | 9.63 ± 2.52 |
| Lung | 12.26 ± 1.45 | 6.51 ± 0.58 |
| Stomach | 3.49 ± 0.37 | 1.93 ± 0.35 |
| Intestinal | 4.94 ± 0.10 | 3.51 ± 0.16 |
| Bone | 3.86 ± 0.97 | 2.92 ± 0.17 |
| Muscle | 1.82 ± 0.19 | 1.25 ± 0.44 |
| Tumor | 26.30 ± 3.92 | 26.11 ± 0.70 |

TABLE 31
Tumor to normal organ ratio of biodistribution of representative FAP
targeting conjugates 24 hours after administration

| | | | | T(% ID)/ |
|---|---|---|---|---|
| | T(% ID/g)/ | T(% ID/g)/ | T(% ID/g)/ | normalized |
| | K(% ID/g) | B(% ID/g) | M(% ID/g) | B (% ID)* |
| DOTAGA-Lys(PEG3-Suc-FAPC(S))-Tyr-NH2 | 0.01 | 33.2 | 30.43 | 3.22 |
| [HO-C18-gGlu-PEG8-PEG8]2-Lys-Lys(PEG8- | 1.6 | 1.5 | 8.97 | 0.34 |
| ESL1-PEG8-DOTAGA)-Lys(PEG3-Suc- | | | | |
| FAPC(S))-Tyr-NH2 | | | | |
| HO-C20-gGlu-PEG8-PEG8-PEG8-PEG8- | 1.5 | 2.4 | 21.97 | 0.24 |
| Lys(DOTAGA)-Lys(PEG3-Suc-FAPC(S))-Tyr- | | | | |
| NH2 | | | | |
| HO-C20-gGlu-PEG8-PEG8-PEG8-PEG8- | 1.4 | 2.4 | 17.62 | 0.26 |
| Lys(ESL1-DOTAGA)-Lys(PEG3-Suc-FAPC(S))- | | | | |
| Tyr-NH2 | | | | |
| HO-C20-gGlu-PEG8-PEG8-PEG8-PEG8- | 0.8 | 5.3 | 22.71 | 0.57 |
| Lys(PEG8-ESL1-PEG8-DOTAGA)-Lys(PEG3- | | | | |
| Suc-FAPC(S))-Tyr-NH2 | | | | |
| HO-C20-gGlu-PEG8-PEG8-PEG8-PEG8- | 1.7 | 2.8 | 28.94 | 0.19 |
| Lys(PEG8-ESL1-OEG-DOTAGA)-Lys(PEG3- | | | | |
| Suc-FAPC(S))-Tyr-NH2 | | | | |
| HO-C20-gGlu-PEG8-PEG8-PEG8-PEG8- | 0.9 | 2.4 | 20.36 | 0.41 |
| Lys(PEG8-ESL1-PEG5-DOTAGA)-Lys(PEG3- | | | | |
| Suc-FAPC(S))-Tyr-NH2 | | | | |
| HO-C20-gGlu-PEG8-PEG8-PEG8-PEG8- | 0.6 | 3.2 | 28.60 | 0.65 |
| Lys(PEG8-ESL1-PEG7-DOTAGA)-Lys(PEG3- | | | | |
| Suc-FAPC(S))-Tyr-NH2 | | | | |
| [HO-C18-gGlu-PEG8-PEG8-PEG8-PEG8]2-Lys- | 2.4 | 1.4 | 12.75 | 0.25 |
| Lys(PEG8-ESL1-PEG8-DOTAGA)-Lys(PEG3- | | | | |
| Suc-FAPC(S))-Tyr-NH2 | | | | |
| [HO-C18-gGlu-PEG8-PEG8-PEG8-PEG8]2-Lys- | 3.3 | 1.0 | 14.43 | 0.24 |
| Lys(PEG8-OEG-PEG8-DOTAGA)-Lys(PEG3- | | | | |
| Suc-FAPC(S))-Tyr-NH2 | | | | |

*Note:
T(% ID)/normalized B (% ID) represents the ratio of average T % ID (T % ID/g * tumor weight) to average B % ID [B % ID/g * blood weight (body weight *7.5%)] normalized by each gram of blood in mice, which is to indicate the ratio of uptake in tumor organ to blood.

TABLE 32
Tumor to normal organ ratio of biodistribution of representative FAP targeting
conjugates 72 hours after administration

| | | | | T(% ID)/ |
|---|---|---|---|---|
| | T(% ID/g)/ | T(% ID/g)/ | T(% ID/g)/ | normalized |
| | K(% ID/g) | B(% ID/g) | M(% ID/g) | B (% ID)* |
| DOTAGA-Lys(PEG3-Suc-FAPC(S))-Tyr-NH2 | N/A | N/A | N/A | N/A |
| HO-C18-gGlu-PEG8-PEG8]2-Lys-Lys(PEG8- | N/A | N/A | N/A | N/A |
| ESL1-PEG8-DOTAGA)-Lys(PEG3-Suc- | | | | |
| FAPC(S))-Tyr-NH2 | | | | |
| HO-C20-gGlu-PEG8-PEG8-PEG8-PEG8- | 2.3 | 7.5 | 24.37 | 0.81 |
| Lys(DOTAGA)-Lys(PEG3-Suc-FAPC(S))-Tyr- | | | | |
| NH2 | | | | |
| HO-C20-gGlu-PEG8-PEG8-PEG8-PEG8- | 2.9 | 10.0 | 36.75 | 1.31 |
| Lys(ESL1-DOTAGA)-Lys(PEG3-Suc-FAPC(S))- | | | | |
| Tyr-NH2 | | | | |
| HO-C20-gGlu-PEG8-PEG8-PEG8-PEG8- | 1.1 | 14.3 | 38.56 | 1.49 |
| Lys(PEG8-ESL1-PEG8-DOTAGA)-Lys(PEG3- | | | | |
| Suc-FAPC(S))-Tyr-NH2 | | | | |
| HO-C20-gGlu-PEG8-PEG8-PEG8-PEG8- | 1.7 | 7.0 | 41.6 | 2.01 |
| Lys(PEG8-ESL1-OEG-DOTAGA)-Lys(PEG3- | | | | |
| Suc-FAPC(S))-Tyr-NH2 | | | | |
| HO-C20-gGlu-PEG8-PEG8-PEG8-PEG8- | 1.6 | 11.0 | >100 | 2.30 |
| Lys(PEG8-ESL1-PEG5-DOTAGA)-Lys(PEG3- | | | | |
| Suc-FAPC(S))-Tyr-NH2 | | | | |
| HO-C20-gGlu-PEG8-PEG8-PEG8-PEG8- | 1.0 | 9.0 | >100 | 2.11 |
| Lys(PEG8-ESL1-PEG7-DOTAGA)-Lys(PEG3- | | | | |
| Suc-FAPC(S))-Tyr-NH2 | | | | |
| [HO-C18-gGlu-PEG8-PEG8-PEG8-PEG8]2-Lys- | 3.4 | 6.5 | >100 | 1.94 |
| Lys(PEG8-ESL1-PEG8-DOTAGA)-Lys(PEG3- | | | | |
| Suc-FAPC(S))-Tyr-NH2 | | | | |
| [HO-C18-gGlu-PEG8-PEG8-PEG8-PEG8]2-Lys- | 4.8 | 2.9 | 20.94 | 0.84 |
| Lys(PEG8-OEG-PEG8-DOTAGA)-Lys(PEG3- | | | | |
| Suc-FAPC(S))-Tyr-NH2 | | | | |

*Note:
T(% ID)/normalized B (% ID) represents the ratio of average T % ID (T % ID/g * tumor weight) to average B % ID [B % ID/g * blood weight (body weight *7.5%)] normalized by each gram of blood in mice, which is to indicate the ratio of uptake in tumor organ to blood.

To evaluate the in vivo biodistribution of the affibody conjugates targeting B7-H-3, each mouse was inoculated with approximately 4×10⁶ B7-H-3 expressing CT26 cells mixed with Matrigel, by subcutaneous injection at rear flank. After inoculation, the xenografted tumors were allowed to grow for ~2-4 weeks till the tumor weight reached to around 100-250 mm³.

Biodistribution studies were performed by administrating ¹⁷⁷Lu labeled complexes of representative compounds of the application at a dose of ~30-100 μCi per mouse (in 100 μL 0.9% NaCl, ~1.1-3.7 MBq, with specific activity 50-500 μCi/nmol) through tail vein injection.

At different time points after the administration, the mice treated in each group were euthanized. The tissues and organs of the animals were collected, excised, weighed, and analyzed in a PerkinElmer 2480 WIZARD2 γ counter, and the percent injected dose (% ID) and % ID/g of each organ or tissue were calculated.

Tables 33-39 below illustrate the biodistribution of the tested ¹⁷⁷Lu-labeled conjugates targeting B7-H3 in tumor bearing mice at 24 and 72 hour after administration.

It was found that, as compared to the comparator conjugate ¹⁷⁷Lu-DOTAGA-Lys(PEG3-Suc-B7H3C(A))-Tyr-NH2, the conjugates including ¹⁷⁷Lu-[HO-C18-gGlu-PEG8-PEG8-PEG8-PEG8]2-Lys-Lys(DOTAGA)-Lys(PEG3-Suc-B7H3C(A))-Tyr-NH2, ¹⁷⁷Lu-[HO-C18-gGlu-PEG8-PEG8-PEG8-PEG8]2-Lys-Lys(ESL1-DOTAGA)-Lys(PEG3-Suc-B7H3C(A))-Tyr-NH2 and ¹⁷⁷Lu-[HO-C18-gGlu-PEG8-PEG8-PEG8-PEG8]2-Lys-Lys(PEG8-ESL1-PEG8-DOTAGA)-Lys(PEG3-Suc-B7H3C(A))-Tyr-NH2 all displayed significantly higher tumor uptake, lower kidney uptake, and improved T/K at 24 h.

In addition, the conjugates with cleavable linkers as described herein also demonstrated superior tumor uptake to normal organ uptake ratios, including tumor to blood and tumor to muscle, over the ones with non-cleavable linkers, for example, cleavable ¹⁷⁷Lu-[HO-C18-gGlu-PEG8-PEG8-PEG8-PEG8]2-Lys-Lys(ESL1-DOTAGA)-Lys(PEG3-Suc-B7H3C(A))-Tyr-NH2 and ¹⁷⁷Lu-[HO-C18-gGlu-PEG8-PEG8-PEG8-PEG8]2-Lys-Lys(PEG8-ESL1-PEG8-DOTAGA)-Lys(PEG3-Suc-B7H3C(A))-Tyr-NH2 vs non-cleavable ¹⁷⁷Lu-[HO-C18-gGlu-PEG8-PEG8-PEG8-PEG8]2-Lys-Lys(DOTAGA)-Lys(PEG3-Suc-B7H3C(A))-Tyr-NH2. Such unexpected further improvements of the biodistribution brought by the cleavable linkers offer the conjugates significantly more favorable profiles concerning therapeutic efficacy and therapeutic safety window.

TABLE 33
Biodistribution of ¹⁷⁷Lu-DOTAGA-Lys
(PEG3-Suc-B7H3C(A))-Tyr-NH2
(ID %/g, Mean ± SD, n ± 3)

| | 4 h | 24 h |
|---|---|---|
| Pancreas | 0.70 ± 0.12 | 0.30 ± 0.03 |
| Kidney | 459.05 ± 30.80 | 460.02 ± 33.83 |
| Blood | 0.19 ± 0.02 | 0.04 ± 0.01 |
| Heart | 0.50 ± 0.10 | 0.73 ± 0.43 |
| Liver | 3.23 ± 0.32 | 1.67 ± 0.17 |
| Spleen | 1.12 ± 0.12 | 1.04 ± 0.19 |
| Lung | 1.10 ± 0.09 | 0.66 ± 0.12 |
| Stomach | 1.29 ± 0.12 | 0.56 ± 0.11 |
| Intestinal | 1.62 ± 0.40 | 1.04 ± 0.18 |
| Bone | 1.10 ± 0.08 | 0.42 ± 0.05 |
| Muscle | 0.27 ± 0.05 | 0.16 ± 0.03 |
| Tumor | 8.92 ± 0.11 | 3.04 ± 0.20 |

TABLE 34
Biodistribution of ¹⁷⁷Lu-[HO-C18-
gGlu-PEG8-PEG8-PEG8-PEG8]
2-Lys-Lys(DOTAGA)-Lys
(PEG3-Suc-B7H3C(A))-Tyr-NH2
(ID %/g, Mean ± SD, n ± 3)

| | 24 h | 72 h |
|---|---|---|
| Pancreas | 4.92 ± 0.31 | 4.38 ± 1.35 |
| Kidney | 26.37 ± 2.29 | 27.12 ± 5.60 |
| Blood | 18.72 ± 1.67 | 9.61 ± 4.16 |
| Heart | 7.10 ± 1.03 | 5.11 ± 1.21 |
| Liver | 12.32 ± 0.70 | 10.44 ± 2.82 |
| Spleen | 9.65 ± 0.39 | 11.97 ± 3.54 |
| Lung | 10.10 ± 2.74 | 14.70 ± 6.61 |
| Stomach | 3.84 ± 0.50 | 2.88 ± 0.71 |
| Intestinal | 6.06 ± 0.56 | 5.78 ± 2.33 |
| Bone | 7.24 ± 0.90 | 5.56 ± 1.06 |
| Muscle | 2.05 ± 0.52 | 1.25 ± 0.44 |
| Tumor | 31.87 ± 7.94 | 29.84 ± 6.04 |

TABLE 35
Biodistribution of ¹⁷⁷Lu-[HO-C18-
gGlu-PEG8-PEG8-PEG8-PEG8]
2-Lys-Lys(ESL1-DOTAGA)-Lys
(PEG3-Suc-B7H3C(A))-Tyr-NH2
(ID %/g, Mean ± SD, n ± 3)

| | 24 h | 72 h |
|---|---|---|
| Pancreas | 5.02 ± 0.91 | 3.46 ± 0.19 |
| Kidney | 24.41 ± 2.41 | 22.65 ± 2.97 |
| Blood | 17.72 ± 5.16 | 7.18 ± 0.94 |
| Heart | 6.19 ± 0.81 | 2.80 ± 2.32 |
| Liver | 8.37 ± 1.21 | 8.02 ± 1.55 |
| Spleen | 9.27 ± 0.70 | 9.87 ± 0.94 |
| Lung | 15.22 ± 7.34 | 5.71 ± 0.79 |
| Stomach | 3.97 ± 0.90 | 1.85 ± 0.34 |
| Intestinal | 6.92 ± 0.33 | 3.51 ± 0.15 |
| Bone | 5.57 ± 0.10 | 3.44 ± 0.07 |
| Muscle | 1.78 ± 0.17 | 1.14 ± 0.11 |
| Tumor | 38.89 ± 4.43 | 44.77 ± 7.60 |

TABLE 36
Biodistribution of ¹⁷⁷Lu-[HO-C18-gGlu-
PEG8-PEG8-PEG8-PEG8]2-Lys-Lys
(PEG8-ESL1-PEG8-DOTAGA)-Lys
(PEG3-Suc-B7H3C(A))-Tyr-NH2
(ID %/g, Mean ± SD, n ± 3)

| | 24 h | 72 h |
|---|---|---|
| Pancreas | 0.84 ± 0.14 | 0.13 ± 0.03 |
| Kidney | 14.05 ± 0.54 | 3.90 ± 1.17 |
| Blood | 1.89 ± 0.24 | 0.12 ± 0.03 |
| Heart | 0.91 ± 0.14 | 0.24 ± 0.22 |
| Liver | 2.28 ± 0.16 | 0.92 ± 0.03 |
| Spleen | 1.90 ± 0.36 | 0.36 ± 0.02 |
| Lung | 1.40 ± 0.14 | 0.26 ± 0.12 |
| Stomach | 0.82 ± 0.10 | 0.09 ± 0.02 |
| Intestinal | 1.28 ± 0.16 | 0.26 ± 0.03 |
| Bone | 1.12 ± 0.07 | 0.36 ± 0.03 |
| Muscle | 0.36 ± 0.03 | 0.06 ± 0.05 |
| Tumor | 13.30 ± 1.68 | 3.56 ± 0.94 |

TABLE 37
Biodistribution of ¹⁷⁷Lu-HO-C20-gGlu-
OEG-OEG-OEG-OEG-OEG-OEG-Lys
(ESL1-DOTAGA)-Lys
(PEG3-Suc-B7H3C(A))-Tyr-NH2
(ID %/g, Mean ± SD, n ± 3)

| | 24 h | 72 h |
|---|---|---|
| Pancreas | 4.32 ± 0.29 | 2.07 ± 0.16 |
| Kidney | 167.95 ± 21.47 | 106.19 ± 5.52 |
| Blood | 11.13 ± 0.69 | 2.70 ± 0.30 |
| Heart | 5.66 ± 0.55 | 2.38 ± 0.33 |
| Liver | 8.58 ± 0.49 | 4.99 ± 0.43 |
| Spleen | 7.55 ± 1.08 | 5.55 ± 0.71 |
| Lung | 8.62 ± 1.88 | 3.40 ± 0.38 |
| Stomach | 4.00 ± 0.26 | 1.90 ± 0.29 |
| Intestinal | 6.49 ± 1.03 | 3.09 ± 0.26 |
| Bone | 3.73 ± 0.14 | 1.71 ± 0.08 |
| Muscle | 1.75 ± 0.05 | 0.88 ± 0.10 |
| Tumor | 38.59 ± 9.13 | 28.91 ± 2.96 |

TABLE 38
Tumor to normal organ ratio of biodistribution of representative B7-H3
targeting conjugates 24 hours after administration

| | T(% ID/g)/ | T(% ID/g)/ | T(% ID/g)/ |
|---|---|---|---|
| | K(% ID/g) | B(% ID/g) | M(% ID/g) |
| DOTAGA-Lys(PEG3-Suc-B7H3C(A))-Tyr-NH2 | 0.01 | 71.17 | 18.59 |
| [HO-C18-gGlu-PEG8-PEG8-PEG8-PEG8]2-Lys- | 1.22 | 1.72 | 15.54 |
| Lys(DOTAGA)-Lys(PEG3-Suc-B7H3C(A))-Tyr-NH2 | | | |
| [HO-C18-gGlu-PEG8-PEG8-PEG8-PEG8]2-Lys-Lys | 1.6 | 2.3 | 21.85 |
| (ESL1-DOTAGA)-Lys(PEG3-Suc-B7H3C(A))-Tyr-NH2 | | | |
| [HO-C18-gGlu-PEG8-PEG8-PEG8-PEG8]2-Lys-Lys | 0.95 | 7.05 | 36.95 |
| (PEG8-ESL1-PEG8-DOTAGA)-Lys(PEG3-Suc-B7H3C(A))- | | | |
| Tyr-NH2 | | | |

TABLE 39
Tumor to normal organ ratio of biodistribution of representative B7-H3
targeting conjugates 72 hours after administration

| | T(% ID/g)/ | T(% ID/g)/ | T(% ID/g)/ |
|---|---|---|---|
| | K(% ID/g) | B(% ID/g) | M(% ID/g) |
| [HO-C18-gGlu-PEG8-PEG8-PEG8-PEG8]2-Lys- | 1.11 | 3.39 | 23.87 |
| Lys(DOTAGA)-Lys(PEG3-Suc-B7H3C(A))-Tyr-NH2 | | | |
| [HO-C18-gGlu-PEG8-PEG8-PEG8-PEG8]2-Lys-Lys | 1.97 | 6.21 | 39.27 |
| (ESL1-DOTAGA)-Lys(PEG3-Suc-B7H3C(A))-Tyr-NH2 | | | |
| [HO-C18-gGlu-PEG8-PEG8-PEG8-PEG8]2-Lys-Lys | 1.04 | 32.12 | 62.82 |
| (PEG8-ESL1-PEG8-DOTAGA)-Lys(PEG3-Suc-B7H3C(A))- | | | |
| Tyr-NH2 | | | |

While the present application has been described with reference to examples, it is to be understood that the scope of the claims should not be limited by the embodiments set forth in the examples but should be given the broadest interpretation consistent with the description as a whole.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the application described and claimed herein.

Sequence Listing

| SEQ | |
|---|---|
| ID | |
| NO. | Sequence |
| 1 | GGGSCSA |
| 2 | GGGSCQA |
| 3 | GGGSCPA |
| 4 | GGGSCHA |
| 5 | EVQLVESGGGLVQPGGSLRLSCAASGSFFRIVAMGWYRQAPGSQRELVATITRGGSTYY |
| | ADSVKGRFTISRDNAKNTLYLQMNSLRAEDTAVYYCNVRVEVPFMQPNDYWGQGTLV |
| | TVSS |
| 6 | GSFFRIVA |
| 7 | ITRGGST |
| 8 | NVRVEVPFMQPNDY |
| 9 | EVQLVESGGGLVQPGGSLRLSCAASGSFFRIVAMGWYRQAPGSQRELVATITRGGSTYYAD |
| | SVKGRFTISRDNAKNTLYLQMNSLRAEDTAVYYCNVRVEVPFMQPNDYWGQGTLVTVSSG |
| | GGSCSA |
| 10 | EVQLVESGGGLVQPGGSLRLSCTGSARPFSNYGMGWFRQAPGNEREFVAAISGGGVYT |
| | DFADSVKGRFTISRDNSKNTLYLQMNSLRAEDTAVYYCARARTARVVSRRHEYDYWG |
| | QGTQVTVSS |
| 11 | ARPFSNYG |
| 12 | ISGGGVYT |
| 13 | ARARTARVVSRRHEYDY |
| 14 | EVQLVESGGGLVQPGGSLRLSCTGSARPFSNYGMGWFRQAPGNEREFVAAISGGGVYT |
| | DFADSVKGRFTISRDNSKNTLYLQMNSLRAEDTAVYYCARARTARVVSRRHEYDYWG |
| | QGTQVTVSSGGGSCSA |
| 15 | EVQLVESGGGLVQPGGSLRLSCTGSARPFSNYGMGWFRQAPGNEREFVAAISGGGVYT |
| | DFADSVKGRFTISRDNSKNTLYLQMNSLRAEDTAVYYCARARTARVVSRRHEYDYWG |
| | QGTQVTVSSGGGSCQA |
| 16 | EVQLVESGGGLVQPGGSLRLSCTGSARPFSNYGMGWFRQAPGNEREFVAAISGGGVYT |
| | DFADSVKGRFTISRDNSKNTLYLQMNSLRAEDTAVYYCARARTARVVSRRHEYDYWG |
| | QGTQVTVSSGGGSCPA |
| 17 | EVQLVESGGGLVQPGGSLRLSCTGSARPFSNYGMGWFRQAPGNEREFVAAISGGGVYT |
| | DFADSVKGRFTISRDNSKNTLYLQMNSLRAEDTAVYYCARARTARVVSRRHEYDYWG |
| | QGTQVTVSSGGGSCHA |
| 18 | QVQLLESGGGLVQPGGSLRLSCAASGFGFGNSLMSWVRRAPGKGLEWVSTIYPRGSFTD |
| | YADSVKGRFTISRDNSRNTLYLQMNSLRAEDTAVYYCATGWGRSSDYDPPGQGTLVTV |
| | SS |
| 19 | GFGFGNSL |
| 20 | IYPRGSFT |
| 21 | ATGWGRSSDYDP |
| 22 | QVQLLESGGGLVQPGGSLRLSCAASGFGFGNSLMSWVRRAPGKGLEWVSTIYPRGSFTD |
| | YADSVKGRFTISRDNSRNTLYLQMNSLRAEDTAVYYCATGWGRSSDYDPPGQGTLVTV |
| | SSGGGSCSA |
| 23 | AEAKX5AKEKIX₁₁ALX₁₄EIIWLPNLTX₁₉X₂₀QIX₂₃AFIAX₂₈LNDDPSQSSELLSEAKKL |
| | X₄₇X₄₈SQ |
| 24 | AEAKFAKEKIKALSEIIWLPNLTYGQIKAFIAKLNDDPSQSSELLSEAKKLSESQ |
| 25 | AEAKFAKEKIKALSEIIWLPNLTYGQIKAFIAKLNDDPSQSSELLSEAKKLSESQGGGCA |
| 26 | AEAKYAKEMRNAYWEIALLPNLTNQQKRAFIRKLYDDPSQSSELLSEAKKLNDSQAPKVDC |

What is claimed is:

1. A compound comprising a structure of formula (I-X): 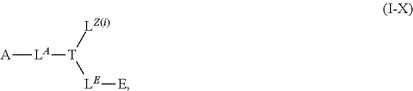
wherein:
(1) L^{Z(i)} is a linker comprising a thiol-reactive group or an amine-reactive group,
(2) T is a branching group which is at least trivalent,
(3) E is an effector moiety, wherein E is conjugated to T via a linker L^{E}, and
(4) A is a potency enhancing moiety, wherein A is conjugated to T via a linker L^{A}.

2. The compound of claim 1, wherein A comprises one or more plasma protein binding groups.

3. The compound of claim 2, wherein the plasma protein is selected from albumin, alpha-1-acid glycoprotein, fetuin, transferrin, IgG, HDL (high-density lipoprotein) or LDL (low-density lipoprotein).

4. The compound of any of claims 1-3, wherein A comprises one or more albumin binding groups.

5. The compound of claim 4, wherein the albumin binding group is selected from the group consisting of unsubstituted or substituted C(O)C₁₋₂₆alkyleneCOOH, unsubstituted or substituted C(O)C₁₋₂₆alkenyleneCOOH, unsubstituted or substituted C(O)C₁₋₂₆alkyl, and unsubstituted or substituted C(O)C₁₋₂₆alkenyl.

6. The compound of claim 5, wherein the albumin binding group is selected from unsubstituted or substituted C(O)C₆₋₂₀alkyleneCOOH, or unsubstituted or substituted C(O)C₇₋₂₁alkyl.

7. The compound of claim 5 or 6, wherein the albumin binding group is selected from the group consisting of C(O)C₇alkyl, C(O)C₈alkyl, C(O)C₉alkyl, C(O)C₁₀alkyl, C(O)C₁₁alkyl, C(O)C₁₂alkyl, C(O)C₁₃alkyl, C(O)C₁₋₄alkyl, C(O)C₁₅alkyl, C(O)C₁₆alkyl, C(O)C₁₇alkyl, C(O)C₁₈alkyl, C(O)C₁₉alkyl, C(O)C₂₀alkyl, C(O)C₂₁alkyl, C(O)C₆alkyleneCOOH, C(O)C₇alkyleneCOOH, C(O)C₈alkyleneCOOH, C(O)C₉alkyleneCOOH, C(O)C₁₀alkyleneCOOH, C(O)C₁₁alkyleneCOOH, C(O)C₁₂alkyleneCOOH, C(O)C₁₃alkyleneCOOH, C(O)C₁₋₄alkyleneCOOH, C(O)C₁₅alkyleneCOOH, C(O)C₁₆alkyleneCOOH, C(O)C₁₇alkyleneCOOH, C(O)C₁₈alkyleneCOOH, C(O)C₁₉alkyleneCOOH, and C(O)C₂₀alkyleneCOOH.

8. The compound of claim 7, wherein the albumin binding group is C(O)C₁₋₄alkyleneCOOH, C(O)C₁₆alkyleneCOOH, or C(O)C₁₈alkyleneCOOH.

9. The compound of any one of claims 4-8, wherein the albumin binding group is selected from CH₃—(CH₂)_{M1}—CO— or COOH—(CH₂)_{N1}—CO—, wherein each of M1 and N1 is independently selected from an integer of 6-22.

10. The compound of claim 9, wherein the albumin binding group is COOH—(CH₂)_{N1}—CO—, and wherein N1 is an integer of 10-18.

11. The compound of claim 9 or 10, wherein the albumin binding group is COOH—(CH₂)₁₆—CO— or COOH—(CH₂)₁₈—CO.

12. The compound of any one of claims 1-11, wherein A comprises a structure of formula (I-Dual-A): 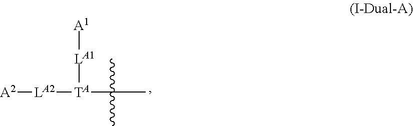
wherein each of A¹ and A² is independently a potency enhancing moiety, A¹ is conjugated to T^{A} via a linker L^{A1}, A² is conjugated to T via a linker L^{A2}, T^{A} is conjugated to T via the linker L^{A}.

13. The compound of claim 12, wherein A¹ and A² are identical.

14. The compound of claim 12 or 13, wherein each of A¹ and A² is independently an albumin binding group.

15. The compound of any one of claims 12-14, wherein each of A¹ and A² is independently selected from the group consisting of unsubstituted or substituted C(O)C₁₋₂₆alkyleneCOOH, unsubstituted or substituted C(O)C₁₋₂₆alkenyleneCOOH, unsubstituted or substituted C(O)C₁₋₂₆alkyl, and unsubstituted or substituted C(O)C₁₋₂₆alkenyl.

16. The compound of claim 15, wherein each of A¹ and A² is independently selected from unsubstituted or substituted C(O)C₆₋₂₀alkyleneCOOH, or unsubstituted or substituted C(O)C₇₋₂₁alkyl.

17. The compound of any one of claims 12-16, wherein each of A¹ and A² is independently selected from the group consisting of C(O)C₇alkyl, C(O)C₈alkyl, C(O)C₉alkyl, C(O)C₁₀alkyl, C(O)C₁₁alkyl, C(O)C₁₂alkyl, C(O)C₁₃alkyl, C(O)C₁₋₄alkyl, C(O)C₁₈alkyl, C(O)C₁₆alkyl, C(O)C₁₇alkyl, C(O)C₁₈alkyl, C(O)C₁₉alkyl, C(O)C₂₀alkyl, C(O)C₂₁alkyl, C(O)C₆alkyleneCOOH, C(O)C₇alkyleneCOOH, C(O)C₈alkyleneCOOH, C(O)C₉alkyleneCOOH, C(O)C₁₀alkyleneCOOH, C(O)C₁₁alkyleneCOOH, C(O)C₁₂alkyleneCOOH, C(O)C₁₃alkyleneCOOH, C(O)C₁₋₄alkyleneCOOH, C(O)C₁₈alkyleneCOOH, C(O)C₁₆alkyleneCOOH, C(O)C₁₇alkyleneCOOH, C(O)C₁₈alkyleneCOOH, C(O)C₁₉alkyleneCOOH, and C(O)C₂₀alkyleneCOOH.

18. The compound of any one of claims 12-17, wherein each of A¹ and A² is independently selected from CH₃—(CH₂)_{M1}—CO— or COOH—(CH₂)_{N1}—CO—, wherein each of M1 and N1 is independently an integer of 6-22.

19. The compound of claim 18, wherein each of A¹ and A² is independently COOH—(CH₂)_{N1}—CO—, wherein N1 is an integer of 6-20.

20. The compound of claim 19, wherein A¹ is COOH—(CH₂)₁₆—CO— and A² is COOH—(CH₂)_{N1}—CO—, wherein N1 is an integer of 8-16.

21. The compound of claim 18, wherein A¹ is COOH—(CH₂)_{N1}—CO— and A² is CH₃—(CH₂)_{M1}—CO—, wherein each of M1 and N1 is independently an integer of 10-18.

22. The compound of claim 20, wherein A¹ is COOH—(CH₂)₁₆—CO— and A² is CH₃—(CH₂)_{M1}—CO—, wherein M1 is an integer of 8-16.

23. The compound of any one of claims 1-22, wherein E comprises a chemotherapeutic agent, a toxin, an immunomodulator, a diagnostic agent, a radionuclide, or a chelating group.

24. The compound of claim 23, wherein E comprises a radionuclide selected from the group consisting of ¹¹C, ¹³N, ¹⁴C, ¹⁵O, ¹⁸F, ⁷⁵Br, ⁷⁶Br, ⁷⁷Br, ¹²³I, ¹²⁴I, ¹²⁵I, ¹³¹I, ³⁵S, ²¹At, ³²P, ³³P, ⁷¹As, ⁷²As, ⁷⁴As, ⁷⁶As, and ⁷⁷As.

25. The compound of claim 23, wherein E comprises a chelating group derived from a chelating agent.

26. The compound of claim 25, wherein the chelating agent is selected from the group consisting of 1,4,7-Triazacyclononane (TACN), 1,4,7-triazacyclononane-triacetic acid (NOTA), 1,4,7-triazacyclononane-N-succinic acid-N′,N″-diacetic acid (NOTASA), 1,4,7-triazacyclononane-N-glutamic acid-N′,N″-diacetic acid (NODAGA), 1,4,7-triazacyclononane-N,N′,N″-tris(methylenephosphonic) acid (NOTP), 1,4,7,10-tetraazacyclododecane ([12]aneN4) (cyclen), 1,4,7,10-tetraazacyclotridecane ([13]aneN4), 1,4,7,11-tetraazacyclotetradecane (iso-cyclam), 1,4,7,10-tetraazacyclododecane-1,4,7,10-tetraacetic acid (DOTA), 2-(1,4,7,10-tetraazacyclododecan-1-yl)acetate (DO1 Å), 2,2′-(1,4,7,10-tetraazacyclododecane-1,7-diyl)diacetic acid (DO2 Å), 2,2′,2″-(1,4,7,10-tetraazacyclododecane-1 ,4,7-triyl)triacetic acid (DO3 Å), 1,4,7,10-tetraazacyclododecane-1,4,7,10-tetra(methanepnosphonic acid) (DOTP), 1,4,7,10-tetraazacyclododecane-1,7-di(methanephosphoriic acid) (DO2P), 1,4,7,10-tetraazacyclododecane-1,4,7-tri(methanephosphonic acid) (DO3P), 1,4,7,10-tetraazacyclo-decane-1-glutamic acid-4,7,10-triacetic acid (DOTAGA), 1,4,7,10-tetraazacyclodecane-1-succinic acid-4,7,10-triacetic acid (DOTASA), 1,4,8,11-tetraazacyclotetradecane ([14]aneN4) (cyclam), 1,4,8,12-tetraazacyclopentadecane ([15]aneN4), 1,5,9,13-tetraazacyclohexadecane ([16]aneN4), 1,4-ethano-1,4,8,11-tetraazacyclo-tetradecane (et-cyclam), 1,4,8,11-tetraazacyclotetradecane-1,4,8,1 1-tetraacetic acid (TETA), 2-(1,4,8,11-tetraazacyclotetradecane-1-y I) acetic acid (TE1 Å), 2,2′-(1,4,8,11-tetraazacyclotetradecane-1,8-diyl)diacetic acid (TE2 Å), 4,11-bis(carboxy methyl)-1,4,8,11-tetraazabicyclo[6.6.2]-hexadecane (CB-TE2 Å), 3,6,10,13,16,19-hexaazabicyclo[6.6.6]icosane (Sar), 1,4,7,10-tetra-(2-carbamoyl-methyl)-cyclododecane (TCMC), N,N′-bis[(6-carboxy-2-pyridil)methyl]-4,13-diaza-18-crown-6 (macropa), phthalocyanines, porphyrins, PCTA (3,6,9,15-tetraazabicyclo[9.3.1]pentadeca-1(15),11,13-triene-3,6,9-triacetic acid), DEPA (7-[2-(biscarboxymethylamino)ethyl]-4,10-biscarboxymethyl-1,4,7,10-tetraazacyclododec-1-yl-acetic acid), DTPA (1,1,4,7,7-diethylenetriaminepentaacetic acid), CHX-DTPA (cyclohexane-1,2-diamineN,N,N′,N′-tetraacetate), BATPA (1,2-bis[2-aminophenoxy]ethane-N,N,N′,N′-tetraacetic acid), TTHA (triethylenetetramineN,N,N′,N″,N″′,N″′-hexaacetic acid), HBED (N,N′-bis[2-hydroxybenzyl]ethylenediamine-N,N′-diacetic acid), EGTA (ethylene glycol bis[2-aminoethyl ether]-N,N,N′,N′-tetraacetic acid), EDTMP (ethylenediamine tetra-[methylene phosphonic acid]), TRAP (triazacyclononate phosphinic acids), SHBED (N,N′-bis[2-hydroxy-5-sulfobenzyl]ethylenediaminediacetic acid), H6Sbbpen(N,N′-bis-[2-hydroxy-5-sulfonylbenzyl]-N,N′-bis[2-methylpyridyl]ethylenediamine), THP (Tris(3,4-hydroxypyridinone), DFO (deferoxamine), FSC (Fusarinine), TAFC (triacetylfusarinine C), FOXE (ferrioxamine E), 6SS (N,N′-bis[2,2-dimethyl-2-mercaptoethyl]ethylenediamine-N,N′-diacetic acid), ECC (ethylenecysteamine cysteine), ECD (ethyl cysteinate dimer), NETA ([2-{4,7-biscarboxymethyl(1,4,7)triazacyclonona-1-yl-ethyl}carbonylmethylamino]acetic acid, THPN (Tetrakis(3-Hydroxy-4-Pyridinone)), H2dedpa (1,2-[{6-(carboxylato-)pyridin-2-yl}methylamino]-ethane), H4octapa (N,N′-bis[6-carboxy-2-pyridylmethyl]-ethylenediamine-N,N′-diacetic acid), H2bispa2 (6,6′-[{9-hydroxy-1,5-bis-(methoxycarbonyl)-2,4-di(pyridin-2-yl)-3,7-diazabicyclo[3.3.1]nonane-3,7-diyl}bis(methylene)]dipicolinic acid), DOTMP(1,4,7,10-Tetraazacyclododecane-1,4,7,10-tetrayl-tetrakis(methylphosphonic acid)), PEPA (1,4,7,10,13-pentaazocyclopentadecane pentaacetic acid), HEHA (1,4,7,10,13,16-hexaazocyclooctadecane hexaacetic acid), H2hox, H2CHXhox, H2octox, H2pyhox, H4neunopa, TETPA, H4pypa, H4py4pa, DTPAm, EGTAm, ampam, Me-3,2-HOPO, 3,4,3-(L1-1,2-HOPO), and macrocyclic tetrapthalimide.

27. The compound of claim 26, wherein the chelating group is derived from DOTA or DOTAGA.

28. The compound of any one of claims 1-27, wherein each of L^{E} and L^{A} is independently selected from a direct bond or a divalent linker.

29. The compound of claim 28, wherein the divalent linker comprises one or more groups selected from the group consisting of R¹NC₁₋₂₀alkyleneNR², R¹NC₁₋₂₀alkenyleneNR², C(O)C₁₋₂₀alkyleneC(O), C(O)C₁₋₂₀alkenyleneC(O), R¹NC₁₋₂₀alkyleneC(O), R¹NC₁₋₂₀alkenyleneC(O), C(O)C₁₋₂₀alkyleneNHR², C(O)C₁₋₂₀alkenyleneNR², C(S)C₁₋₂₀alkyleneC(S), C(S)C₁₋₂₀alkenyleneC(S), C(S)C₁₋₂₀alkyleneC(O), C(S)C₁₋₂₀alkenyleneC(O), C(O)C₁₋₂₀alkyleneC(S), C(O)C₁₋₂₀alkenyleneC(S), C(O)C₁₋₂₀alkyleneO, C(O)C₁₋₂₀alkenyleneO, R¹NC₁₋₂₀alkenyleneC(S), C(S)C₁₋₂₀alkyleneNR² and C(S)C₁₋₂₀alkenyleneNR², each of which is independently and optionally interrupted by one or more of S, O, NH, N(C₁₋₆alkyl), C(O), C(O)NH, NHC(O), C(S)NH, NHC(S), NHC(O)NH, NHC(S)NH, NHC(NH), NHC(NC₁₋₄alkyl), C(NH)NH, C(NC₁₋₄alkyl)NH, NC₄₋₁₀cycloalkyl, C₄₋₁₀heterocycloalkyl, C₆₋₁₀aryl and C₅₋₁₀heteroaryl, and wherein each alkyl, alkylene and alkenylene is optionally substituted with one or more substituents selected from halo, COOH, C₁₋₆alkyl, OH, OC₁₋₆alkyl, SH, SC₁₋₆alkyl, NR³R⁴, C₁₋₄alkyleneOH, C₁₋₄alkyleneOC₁₋₄alkyl and C₁₋₄alkyleneNR³R⁴, and each R¹, R², R³ and R⁴ is independently selected from H and C₁₋₄alkyl.

30. The compound of any one of claims 28-29, wherein the divalent linker comprises one or more amino acid residues.

31. The compound of claim 30, wherein the amino acid residue is derived from a naturally occurring amino acid selected from the group consisting of alanine (Ala, A), arginine (Arg, R), asparagine (Asn, N), aspartic acid (Asp, D), glutamine (Gln, Q), glutamic acid (Glu, E), γGlu, glutamine (Gln), glycine (Gly, G), histidine (His, H), isoleucine (Ile, I), leucine (Leu, L), lysine (Lys, K), εLys, methionine (Met, M), phenylalanine (Phe, F), proline (Pro, P), serine (Ser, S), threonine (Thr, T), tryptophan (Trp, W), tyrosine (Tyr, Y), valine (Val, V), pyrrolysine (Pyl, O), pyrroline-carboxy-lysine (PCL), gamma-carboxyglutamic acid (Gla), and a D enantiomer thereof.

32. The compound of claim 30, wherein the amino acid residue is derived from a unnaturally occurring amino acid selected from the group consisting of NR⁵C₅alkyleneC(O) (Ahx), L-Cysteic acid (Cya), norleucine (Nle), norvaline (Nva), 2-aminooctanoic acid (Aoc), 2-naphthylalanine (2-Nal), 3-(trifluoromethyl)phenylalanine (TFP), homophenylalanine (hPhe), cyclohexylalanine (Cha), 1-naphthylalanine (1-Nal), 4-benzoyl-L-phenylalanine (Bpa), 2-methoxy-4-vinylphenylalanine (MvF), 4-fluorophenylalanine (4-F-Phe), 4-phenyl-2,3-dihydroxy-6-nitrophenylalanine (pNIPA), 2-(2-naphthyl)alanine (2-Nal-ala), 4-(4-propoxyphenyl)alanine (Ppa), 4-carboxyphenylalanine (4-CPA), 4-butylphenylalanine (Bua), 2-nitrophenylalanine (2-Npa), 4-azidophenylalanine (4-AzF), 2-(4-nitrophenyl)ethylalanine (2-Npe), 3-iodo-L-tyrosine (Ity), and 5,5,5-trifluoroleucine (TFL), and a D enantiomer thereof.

33. The compound of any one of claims 28-32, wherein the divalent linker comprises one of more moieties of —(OCH₂CH₂)ₙ—, wherein n is an integer of 1-28.

34. The compound of claim 33, wherein the divalent linker comprises one or more moieties each independently selected from the group consisting of —(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), —(C₂alkyleneO)₆C₂alkyleneC(O) (PEG6), —(C₂alkyleneO)₆C₂alkyleneC(O) (PEG8), and —(C₂alkyleneO)₆C₂alkyleneC(O) (PEG12).

35. The compound of any one of claims 28-34, wherein at least one of L^{E} and L^{A} is a cleavable linker.

36. The compound of claim 35, wherein the cleavable linker comprises one or more cleavable moieties selected from the group consisting of S—S, C(O)O, OC(O), OC(O)O, OC(O)NH, NHC(O)O, SC(O), C(O)S, C═NNH, C═NO, NC₁₋₄alkylC(O)O, NHOC(O)NH, NC₁₋₄alkylOC(O), and an enzymatically cleavable peptide sequence.

37. The compound of claim 35 or 36, wherein the cleavable linker comprises one or more groups selected from the group consisting of R⁵NC₁₋₂₀alkyleneNR⁶, R⁵NC₁₋₂₀alkenyleneNR⁵, C(O)C₁₋₂₀alkyleneC(O), C(O)C₁₋₂₀alkenyleneC(O), R⁵NC₁₋₂₀alkyleneC(O), R⁵NC₁₋₂₀alkenyleneC(O), C(O)C₁₋₂₀alkyleneNR⁶, C(O)C₁₋₂₀alkenyleneNR⁶, C(S)C₁₋₂₀alkyleneC(S), C(S)C₁₋₂₀alkenyleneC(S), C(S)C₁₋₂₀alkyleneC(O), C(S)C₁₋₂₀alkenyleneC(O), C(O)C₁₋₂₀alkyleneC(S), C(O)C₁₋₂₀alkenyleneC(S), SC₁₋₂₀alkyleneS, SC₁₋₂₀alkenyleneS, SC₁₋₂₀alkyleneNR⁶, SC₁₋₂₀alkenyleneNR⁶, R⁵NC₁₋₂₀alkyleneS, R⁵NC₁₋₂₀alkenyleneS, R⁵NC₁₋₂₀alkyleneO, R⁵NC₁₋₂₀alkenyleneO, OC₁₋₂O alkyleneNR⁶, OC₁₋₂₀alkenyleneNR⁶, SC₁₋₂₀alkyleneO, SC₁₋₂₀alkenyleneO, OC₁₋₂₀alkyleneS, and OC₁₋₂₀alkenyleneS, C(O)C₁₋₂₀alkyleneO, C(O)C₁₋₂₀alkenyleneO, OC₁₋₂₀alkyleneC(O), OC₁₋₂₀alkenyleneC(O), C(O)C₁₋₂₀alkyleneS, C(O)C₁₋₂₀alkenyleneS, SC₁₋₂₀alkyleneC(O), SC₁₋₂₀alkenyleneC(O), R⁵NC₁₋₂₀alkyleneC(S), R⁵NC₁₋₂₀alkenyleneC(S), C(S)C₁₋₂₀alkyleneNR⁶, C(S)C₁₋₂₀alkenyleneNR⁶, C(S)C₁₋₂₀alkyleneO, C(S)C₁₋₂O alkenyleneO, OC₁₋₂₀alkyleneC(S), OC₁₋₂₀alkenyleneC(S), SC₁₋₂₀alkyleneC(S), SC₁₋₂₀alkenyleneC(S), OC₁₋₂₀alkyleneO, OC₁₋₂₀alkenyleneCO, SC₁₋₂₀alkyleneS, or SC₁₋₂₀alkenyleneS, each of which is independently and optionally interrupted by one or more of S—S, C(O)O, OC(O), OC(O)O, OC(O)NH, NHC(O)O, SC(O), C(O)S, NC₁₋₄alkylC(O)O, NHOC(O)NH, NC₁₋₄alkylOC(O), C═NNH, C═NNH₂, C═NOH, C═NO, NH—NH, NH—NC₁₋₄alkyl, NC₁₋₄alkyl-NH, NC₁₋₄alkylNC₁₋₄alkyl, S, O, NH, N(C₁₋₆alkyl), C(O), C(O)NH, NHC(O), NHC(O)NH, NHC(S)NH, C(S)NH, NHC(S), NHC(NH), NHC(NC₁₋₄alkyl), C(NH)NH, C(NC₁₋₄alkyl)NH, NC₄₋₁₈cycloalkyl, C₄₋₁₀heterocycloalkyl, C₆₋₁₀aryl and C₅₋₁₀heteroaryl, and
wherein each alkyl, alkylene or alkenylene is optionally substituted with one or more substituents selected from halo, COOH, C₁₋₆alkyl, OH, OC₁₋₆alkyl, SH, SC₁₋₆alkyl, NR⁷R⁷, C₁₋₄alkyleneOH, C₁₋₄alkyleneOC₁₋₄alkyl and C₁₋₄alkyleneNR⁷R⁸, and
each of R⁵, R⁶, R⁷ and R⁸ is independently selected from H and C₁₋₄alkyl.

38. The compound of any one of claims 35-37, wherein the cleavable linker comprises or is a structure of Formula (I-CL3):
—Y¹—W¹—Y²—W²—Y³—W³—Y⁴—W⁴—Y⁵—W⁵—Y⁶—, (I-CL3)
wherein each of W¹, W², W³, W⁴, or W⁵ is independently a bond, or unbranched or branched C₁₋₆alkylene;
wherein each of Y¹, Y², Y³, Y⁴, Y⁵, Y⁶ is independently a bond, O, S, S—S, C(O)O, OC(O), OC(O)O, OC(O)NR^{Y}, NR^{Y}C(O)O, NR^{Y}, C(O), C(O)NR^{Y}, NR^{Y}C(O), NR^{Y}C(O)NR^{Y}, C₅₋₇ cycloalkyl, C₄₋₆heterocycloalkyl, or CH(R^{AA}),
wherein each R^{Y} is independently H, C₁₋₆alkyl, C₁₋₆alkyl substituted with one or more independently selected halogen, C₁₋₆alkyleneOH, C₁₋₆alkyleneNR⁷R⁸, C₁₋₅alkyleneCOOH, or C₁₋₄alkyleneOC₁₋₄alkyl; and
wherein R^{AA} is a side chain of a naturally occurring amino acid;
provided that at least one of Y¹, Y², Y³, Y⁴, Y⁵, Y⁶ is not a bond.

39. The compound of claim 38, wherein at least one of Y¹, Y², Y³, Y⁴, Y⁵, Y⁶ is C(O)O, OC(O), OC(O)O, OC(O)NR^{Y}, or NR^{Y}C(O)O.

40. The compound of any one of claims 35-39, wherein the cleavable linker comprises one or more groups comprising a structure of Formula (I-CL1) 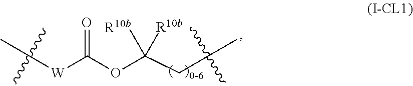
wherein W is selected from C(R^{10a})₂, N(R^{10a}) or 0,
each R^{10a} is independently H, C₁₋₆alkyl, C₁₋₆alkyl substituted with one or more independently selected halogen, C₁₋₆alkyleneOH, C₁₋₆alkyleneNR⁷R⁸, C₁₋₅alkyleneCOOH, C₁₋₄alkyleneOC₁₋₄alkyl, or the side chain of a naturally occurring amino acid, or two R^{10a} groups, together with the carbon atom to which they are attached, form a C₃₋₁₀cycloalkyl or C₃₋₁₀heterocycloalkyl;
each R^{10b} is independently H, C₁₋₆alkyl, C₁₋₆alkyl substituted with one or more independently selected halogen, C₁₋₆alkyleneOH, C₁₋₆alkyleneNR⁷R⁸, C₁₋₅alkyleneCOOH, C₁₋₄alkyleneOC₁₋₄alkyl, or two R^{10b} groups, together with the carbon atom to which they are attached, form a C₃₋₁₀cycloalkyl or C₃₋₁₀heterocycloalkyl; and
R⁷ and R⁸ are each independently H, or C₁₋₄alkyl.

41. The compound of claim any one of claims 35-40, wherein the cleavable linker comprises one or more groups selected from the group consisting of

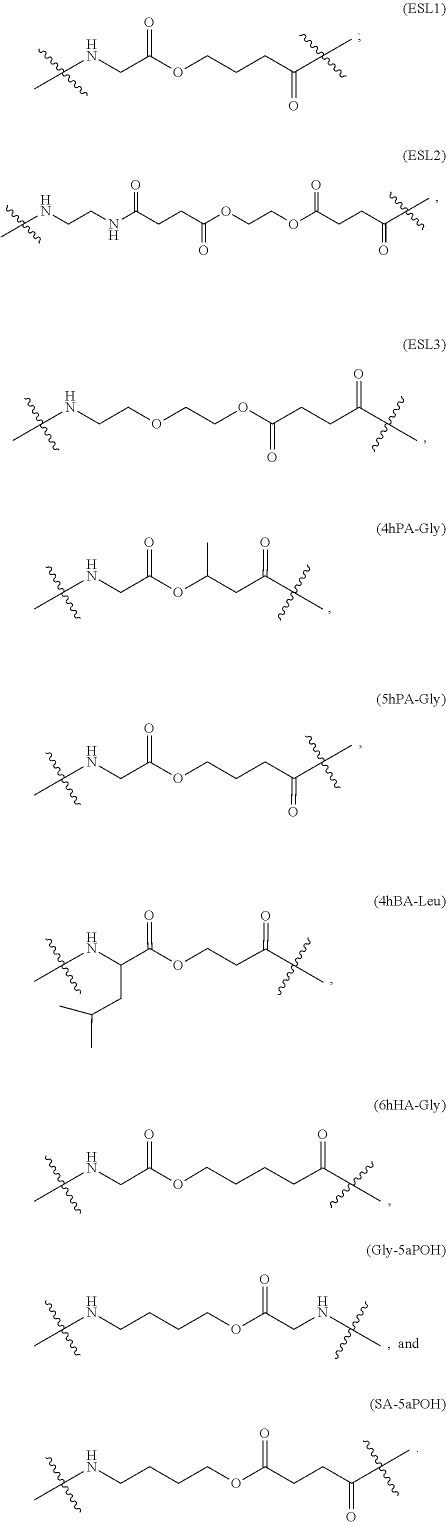

42. The compound of claim 38, wherein at least one of Y¹, Y², Y³, Y⁴, Y⁵, Y¹ is —S—S—.

43. The compound of any one of claims 35-38 or 42, wherein the cleavable linker comprises one or more groups having a structure of Formula (I-CL2): 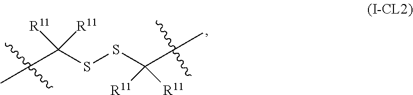
and
wherein each R¹¹ is independently selected from H or C₁₋₄alkyl.

44. The compound of claim 43, wherein the cleavable linker comprises one or more groups selected from the group consisting of

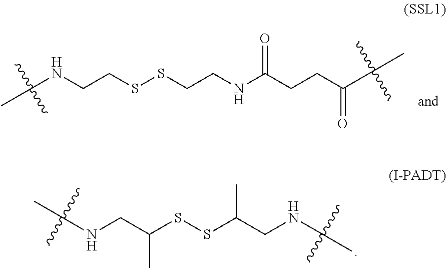 [SMILES: *NCCSSCCNC(=O)CCC(*)=O ; *NCC(C)SSC(C)CN*]

45. The compound of claim 36, wherein the enzymatically cleavable peptide sequence is selected from the group consisting of Met-Val-Lys, Ala-Val, Ala-Pro, Gly-Pro, Ser-Ser, Ser-Gly, Gly-Ala and Ser-Lys.

46. The compound of any one of claims 1-38, wherein L^{Z(i)} comprises a thiol-reactive group.

47. The compound of claim 46, wherein the thiol-reactive group is selected from a thiol-reactive double bond, a thiol-reactive triple bond, or a leaving group containing moiety.

48. The compound of claim 46 or 47, wherein the thiol-reactive group is selected from the group consisting of

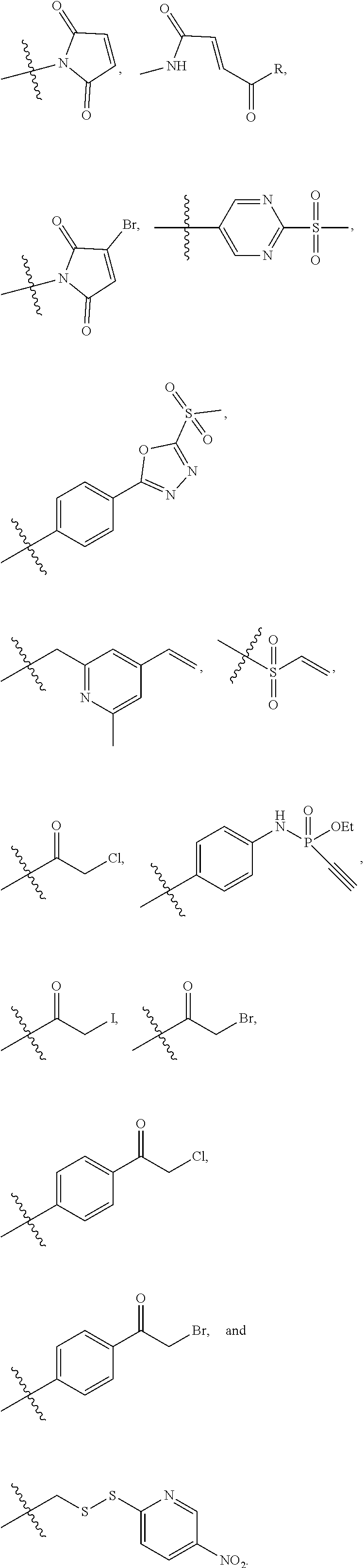

49. The compound of any one of claims 1-38, wherein L^{Z(i)} comprises an amine-reactive group.

50. The compound of claim 49, wherein the amine-reactive group is selected from the group consisting of

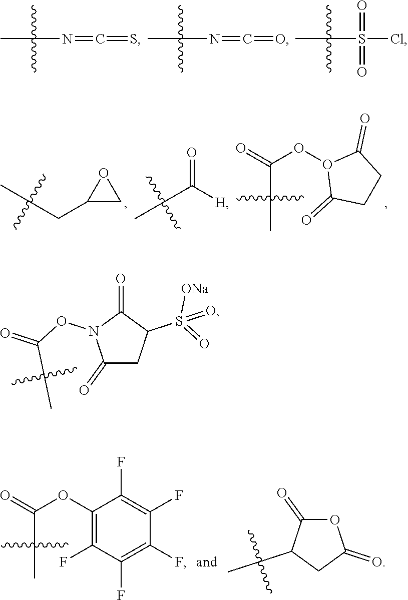

51. The compound of any one of claims 46-50, wherein L^{Z(i)} further comprises one or more groups selected from an R¹NC₁₋₂₀alkyleneNR², R¹NC₁₋₂₀alkenyleneNR² C(O)C₁₋₂₀alkyleneC(O), C(O)C₁₋₂₀alkenyleneC(O), R¹NC₁₋₂₀alkyleneC(O), R¹NC₁₋₂₀alkenyleneC(O), C(O)C₁₋₂₀alkyleneNHR², C(O)C₁₋₂₀alkenyleneNR², C(S)C₁₋₂₀alkyleneC(S), C(S)C₁₋₂₀alkenyleneC(S), C(S)C₁₋₂₀alkyleneC(O), C(S)C₁₋₂₀alkenyleneC(O), C(O)C₁₋₂₀alkyleneC(S), C(O)C₁₋₂₀alkenyleneC(S), C(O)C₁₋₂₀alkyleneO, C(O)C₁₋₂₀alkenyleneO, R¹NC₁₋₂₀alkenyleneC(S), C(S)C₁₋₂₀alkyleneNR² or C(S)C₁₋₂₀alkenyleneNR², each of which is independently and optionally interrupted by one or more of S, O, NH, N(C₁₋₆alkyl), C(O), C(O)NH, NHC(O), C(S)NH, NHC(S), NHC(O)NH, NHC(S)NH, NHC(NH), NHC(NC₁₋₄alkyl), C(NH)NH, C(NC₁₋₄alkyl)NH, NC₄₋₁₀cycloalkyl, C₄₋₁₀heterocycloalkyl, C₆₋₁₀aryl and C₅₋₁₀heteroaryl, and each alkyl, alkylene and alkenylene is optionally and independently substituted with one or more substituents selected from halo, COOH, C₁₋₆alkyl, OH, OC₁₋₆alkyl, SH, SC₁₋₆alkyl, NR³R⁴, C₁₋₄alkyleneOH, C₁₋₄alkyleneOC₁₋₄alkyl and C₁₋₄alkyleneNR³R⁴, and wherein each R¹, R², R³ and R⁴ is independently selected from H and C₁₋₄alkyl.

52. The compound of any one of claims 46-51, wherein L^{Z(i)} comprises one or more amino acid residues.

53. The compound of claim 52, wherein the amino acid residue is derived from a naturally occurring amino acid selected from the group consisting of alanine (Ala, A), arginine (Arg, R), asparagine (Asn, N), aspartic acid (Asp, D), glutamine (Gln, Q), glutamic acid (Glu, E), γGlu, glutamine (Gln), glycine (Gly, G), histidine (His, H), isoleucine (Ile, I), leucine (Leu, L), Lysine (Lys, K), εLys, methionine (Met, M), phenylalanine (Phe, F), proline (Pro, P), serine (Ser, S), threonine (Thr, T), tryptophan (Trp, W), tyrosine (Tyr, Y), valine (Val, V), pyrrolysine (Pyl, O), pyrroline-carboxy-lysine (PCL), gamma-carboxyglutamic acid (Gla), and a D enantiomer thereof.

54. The compound of claim 52, wherein the amino acid residue is derived from a unnaturally occurring amino acid selected from the group consisting of NR⁵C₅alkyleneC(O) (Ahx), L-Cysteic acid (Cya), norleucine (Nle), norvaline (Nva), 2-aminooctanoic acid (Aoc), 2-naphthylalanine (2-Nal), 3-(trifluoromethyl)phenylalanine (TFP), homophenylalanine (hPhe), cyclohexylalanine (Cha), 1-naphthylalanine (1-Nal), 4-benzoyl-L-phenylalanine (Bpa), 2-methoxy-4-vinylphenylalanine (MvF), 4-fluorophenylalanine (4-F-Phe), 4-phenyl-2,3-dihydroxy-6-nitrophenylalanine (pNIPA), 2-(2-naphthyl)alanine (2-Nal-ala), 4-(4-propoxyphenyl)alanine (Ppa), 4-carboxyphenylalanine (4-CPA), 4-butylphenylalanine (Bua), 2-nitrophenylalanine (2-Npa), 4-azidophenylalanine (4-AzF), 2-(4-nitrophenyl)ethylalanine (2-Npe), 3-iodo-L-tyrosine (Ity), or 5,5,5-trifluoroleucine (TFL), and a D enantiomer thereof.

55. The compound of any one of claims 46-54, wherein L^{Z(i)} comprises one of more moieties of —(OCH₂CH₂)ₙ—, wherein n is an integer of 1-28.

56. The compound of claim 55, wherein L^{Z(i)} comprises one or more moieties each independently selected from the group consisting of —(C₂alkyleneO)₂C₁alkyleneC(O) (OEG), —(C₂alkyleneO)C₂alkyleneC(O) (PEG1), —(C₂alkyleneO)₃C₂alkyleneC(O) (PEG3), —(C₂alkyleneO)₆C₂alkyleneC(O) (PEG6), —(C₂alkyleneO)₆C₂alkyleneC(O) (PEG8), and —(C₂alkyleneO)₆C₂alkyleneC(O) (PEG12).

57. The compound of any one of claims 1-56, wherein T comprises one or more amino acid residues each independently derived from Lys, ornithine (Orn), homo-lysine, 2,3-diaminopropionic acid (Dap), 2,4-diaminobutyric acid (Dab), cysteine, homo-cysteine, glutamine, glutamic acid, asparagine, aspartic acid, 3,5-bis(aminomethyl)benzoic acid (Bab), 4-aminomethylphenylalaline (Amp), 4R-4-aminoproline (Apr), 4-(2-aminoethoxy)phenylalanine, 4-aminopiperidine-4-carboxylic acid (Apc), 2-((1,3-diaminopropan-2-yl)oxy)acetic acid (Dpa), or a D enantiomer thereof.

58. The compound of claim 57, wherein T comprises a first terminal functionality, a second terminal functionality and a third terminal functionality, which are the same or different and bind to L^{Z(i)}, L^{A} (or alternatively A), and L^{E} (or alternatively E), respectively to independently form an amide group, an amine group, a urea group, a thioether group, a thiourea group or a thioamide group.

59. The compound of claim 57 or 58, wherein T binds to L^{A} (or alternatively A), and/or L^{E} (or alternatively E) to further form an ester group, a thioester group, a carbonate group, a carbamate group, a disulfide bond, a hydrazone group, or an oxime group.

60. The compound of any one of claims 1-59, wherein the compound is selected from any one of Table 2.

61. A conjugate, comprising the structure of formula (I): 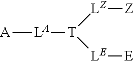
wherein:
(1) T is a branching group which is at least trivalent,
(2) Z is a target binding moiety specifically binding to a target expressed on a cell, wherein Z has a molecular size of less than 66 kDa and is conjugated to T via a linker L^{Z},
(3) E is an effector moiety, wherein E is conjugated to T via a linker L^{E}, and
(4) A is a potency enhancing moiety, wherein A is conjugated to T via a linker L^{A}, wherein A, L^{A}, E, L^{E}, and T are as defined in any one of claims 1-60.

62. The conjugate of claim 61, wherein the potency enhanced by A includes improved binding affinity against the tumor antigen that Z specifically binds to, extended in vivo half-life, increased tumor uptake, and/or enhanced in vivo activity.

63. The conjugate of claim 61 or 62, wherein E comprises a chelating group complexed with a radionuclide.

64. The conjugate of claim 63, wherein the radionuclide is a radioactive isotope selected from the group consisting of As, Se, K, Sc, Ti, Cr, Mn, Fe, Co, Ni, Cu, Ga, Ge, Rb, Sr, Y, Zr, Nb, Tc, Rh, Pd, In, Sn, Sb, Zn, Ta, W, Re, Os, Ir, Pt, Au, Hg, Tl, Pb, Bi, Po, Fr, Pm, a lanthanide, an actinide, Mg, Al, Ca, Cd, and Ba.

65. The conjugate of claim 64, wherein the radioactive isotope is lanthanide, and wherein the lanthanide is Lu, Sm, Ho, or Tb.

66. The conjugate of claim 64, wherein the radioactive isotope is actinide, and wherein the actinide is Ac, Th, or U.

67. The conjugate of any one of claims 63-66, wherein the one or more radionuclides are each independently selected from the group consisting of ⁹⁹Tc, ^{99m}Tc, ¹⁸⁸Re, ¹⁸⁶Re, ¹⁵³Sm, ⁶⁶Ga, ⁶⁷Ga, ⁶⁸Ga, ¹¹¹In, ¹²³In, ⁵⁹Fe, ⁶³Zn, ⁵²Fe, ⁵²Mn, ⁴⁵Ti, ⁶⁰Cu, ⁶¹Cu, ⁶⁷Cu, ⁶⁴Cu, ⁶²Cu, ⁸²Rb, ^{195m}Pt, ^{191m}Pt, ^{193m}Pt, ^{117m}Sn, ⁸⁹Zr, ¹⁷⁷Lu, ¹⁸F, ¹⁸⁸Re, ¹⁸⁶Re, ¹⁵³Sm, ⁶⁶Ho, ⁸⁶Y, Y, ⁸⁹ Sr, ¹⁵³Gd, ¹⁹Gd, ²²⁵Ac, ²¹²Bi, ²¹³Bi, ¹⁹⁸Au, ¹⁹⁹Au, ^{193m}Pt, ¹⁹⁷Pt, ¹⁰³Pd, ¹⁰⁹Pd, ¹⁰⁵Rh, ^{101m}Rh ^{103m}Rh, ²²³Ra, ²²⁴Ra, ⁹⁷Ru, ²²⁷Th, ²²⁹Th, ¹⁶¹Tb, ¹⁴⁹Tb, ²⁰³Pb, ²¹²Pb, ²⁰¹TI, ¹¹⁹Sb, ^{58m}Co, ⁵⁵Co, ⁵⁷Co ⁴⁷SC, ¹⁴⁹Pm, ¹⁴²Pr, ¹⁶¹Ho, ¹⁶⁶Ho, ¹⁷⁵Yb, and ⁵¹Cr.

68. The conjugate of any one of claims 63-67, wherein E is ¹⁷⁷Lu-DOTA-, ¹⁷⁷Lu-DOTAGA-, ²²⁵Ac-DOTA-, or ²²⁵Ac-DOTAGA-.

69. The conjugate of any one of claims 61-68, wherein the target Z specifically binds to is a tumor antigen expressed on a tumor cell.

70. The conjugate of claim 69, wherein the target is selected from the group consisting of Carbonic Anhydrase IX (CAIX), B7 Homolog 3 (B7-H3), Guanylyl cyclase C (GCC), prostate specific membrane antigen (PSMA), fibroblast activation protein alpha (FAP-alpha, FAP), Epidermal Growth Factor Receptor (EGFR), Human Epidermal Growth Factor Receptor-2 (HER-2), Receptor tyrosine-protein kinase erbB-3 (HER-3), Platelet-derived growth factor receptor beta (PDGF-Rβ), Programmed death-ligand 1 (PD-L1), Tumour Necrosis Factor alpha (TNF-α), Vascular endothelial growth factor receptor 2 (VEGFR-2), Vascular Endothelial Growth Factor (VGFR), Vascular Endothelial Growth Factor Receptor (VEGFR), Claudin 18.2, Glucagon-like Peptide-1 Receptor (GLP-1R), Glucose-dependent Insulinotropic Polypeptide Receptor (GIP-R), Cholecystokinin-2 Receptor (CCK2R), Gastrin Releasing Peptide Receptor (GRPR), Somatostatin Receptor 2 (SSTR2), Neurotensin Receptor 1 (NTR1), Neuropeptide Y Receptor Type 1 (Y1R), Nectin-4, Epithelial Cell Adhesion Molecule (EpCAM), and Insulin-like Growth Factor-1 (IGF-1) Receptor.

71. The conjugate of any one of claims 61-70, wherein Z comprises an antibody or antibody mimetic selected from the group consisting of a single domain antibody (sdAb), a fragment antigen-binding region (Fab), a single-chain variable fragment (scFv), an affibody, a DARPin (an acronym for designed ankyrin repeat proteins), an Anticalin, an Avimer (avidity multimer), and a Versabody.

72. The conjugate of any one of claims 61-71, wherein L^{Z} is covalently conjugated with a Cys or Lys amino acid residue of Z.

73. The conjugate of claim 72, wherein L^{Z} is covalently conjugated with a Cys amino acid residue of Z by a structure of L^{Z} selected from the group consisting of

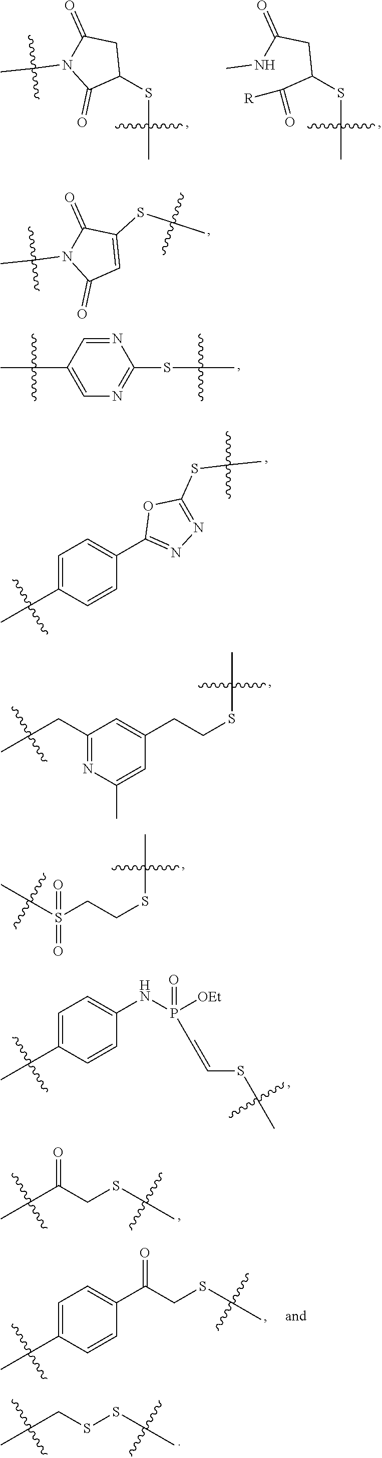

74. The conjugate of claim 72, wherein L^{Z} is covalently conjugated with a Lys amino acid residue of Z by a structure L^{Z} selected from the group consisting of

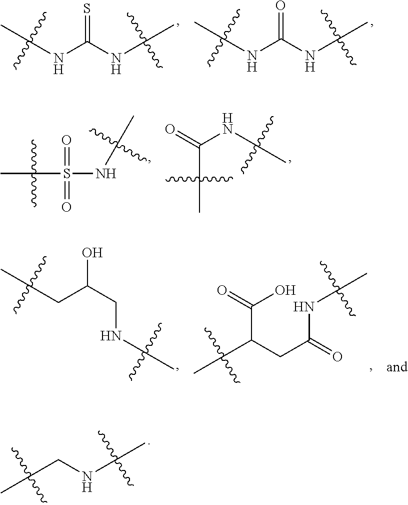

75. The conjugate of any one of claims 61-74, wherein Z comprises a structure of formula (XI):
B—P (XI),
wherein:
B is an antibody or antibody mimetic selected from a single domain antibody (sdAb), a fragment antigen-binding region (Fab), a single-chain variable fragment (scFv), an affibody, a DARPin (an acronym for designed ankyrin repeat proteins), an Anticalin, an Avimer (avidity multimer), or a Versabody; and
P is null or comprises a structure of formula (XII):
X—J—Y (XII),
wherein X is null or an amino acid linker, J is a Cys residue, and Y is null or an extension moiety, and wherein X or Y do not contain an amino acid residue that is identical to J.

76. The conjugate of claim 75, wherein B is a single domain antibody specifically binding to a tumor antigen selected from the group consisting of Guanylyl cyclase C (GCC), prostate specific membrane antigen (PSMA), fibroblast activation protein alpha (FAP-alpha, FAP), Epidermal Growth Factor Receptor (EGFR), Human Epidermal Growth Factor Receptor-2 (HER-2), Vascular Endothelial Growth Factor (VGFR), Vascular Endothelial Growth Factor Receptor (VEGFR), Claudin 18.2, Glucagon-like Peptide-1 Receptor (GLP-1R), Glucose-dependent Insulinotropic Polypeptide Receptor (GIP-R), Cholecystokinin-2 Receptor (CCK2R), Gastrin Releasing Peptide Receptor (GRPR), Somatostatin Receptor 2 (SSTR2), Neurotensin Receptor 1 (NTR1), Neuropeptide Y Receptor Type 1 (Y1R), Nectin-4, Epithelial Cell Adhesion Molecule (EpCAM), and Insulin-like Growth Factor-1 (IGF-1) Receptor.

77. The conjugate of claim 75 or 76, wherein B is a single domain antibody comprising:
(1) a CDR1 comprising an amino acid sequence as set forth in SEQ ID NO. 6, 11, or 19, or an amino acid sequence with one to three amino acid alterations as compared to SEQ ID NO. 6, 11, or 19,
(2) a CDR2 comprising an amino acid sequence as set forth in SEQ ID NO. 7, 12, or 20, or an amino acid sequence with one to three amino acid alterations as compared to SEQ ID NO. 7, 12, or 20, and
(3) a CDR3 comprising an amino acid sequence as set forth in SEQ ID NO. 8, 13, or 21, or an amino acid sequence with one to three amino acid alterations as compared to SEQ ID NO. 8, 13, or 21.

78. The conjugate of claim 77, wherein the sdAb comprises an amino acid sequence having at least 80% identity with SEQ ID NO. 5, 10, or 18.

79. The conjugate of claim 75, wherein B is an affibody specifically binding to a tumor antigen selected from Carbonic Anhydrase IX (CAIX), Epidermal growth factor receptor (EGFR), Receptor tyrosine-protein kinase erbB-2 (HER-2), Receptor tyrosine-protein kinase erbB-3 (HER-3), Insulin-like growth factor 1 (IGF-1R), Platelet-derived growth factor receptor beta (PDGF-Rβ), Programmed death-ligand 1 (PD-L1), Tumour Necrosis Factor alpha (TNF-α), Vascular endothelial growth factor receptor 2 (VEGFR-2) or B7 Homolog 3 (B7-H3).

80. The conjugate of claim 79, wherein B is an affibody comprising an amino acid sequence as set forth in SEQ ID NO. 23:
(SEQ ID NO. 23)
AEAKXAKEKIX₁₁ALX₁₄EIIWLPNLTX₁₉X₂₀QIX₂₃AFIAX₂₈LNDDP
SQSSELLSEAKKLX₄₇X₄₈SQ,
wherein
X₅ is selected from Phe or Tyr;
X₁₁ is selected from Lys, Asn or Ala;
X₁₄ is selected from Ser or Gly;
X₁₉ is selected from Tyr or His;
X₂₀ is selected from Gly or Asp;
X₂₃ is selected from Lys or Met;
X₂₈ is selected from Lys or Ala;
X₄₇ is selected from Ser or Asn;
X₄₈ is selected from Glu or Asp;
X₅₁ is selected from null or Gly;
X₅₂ is selected from null or Gly;
X₅₃ is selected from null or Gly;
X₅₄ is selected from Cys or Lys; and
X₅₅ is selected from null or Ala.

81. The conjugate of claim 80, wherein B is an affibody comprising an amino acid sequence having at least 80% identity with SEQ ID NO. 24.

82. The conjugate of any one of claims 75-81, wherein P is at C-terminus of B.

83. The conjugate of any one of claims 75-81, wherein P comprises no more than 20 amino acid residues.

84. The conjugate of any one of claims 75-83, wherein X comprises an amino acid sequence selected from (GGS)ₙ, (GSG)ₙ, (GGGS)ₙ or (GGGGS)ₙ, (GGG)ₙ, (EAAAK)ₙ, and n is any integer from 0-3.

85. The conjugate of claim 84, wherein X comprises an amino acid sequence selected from GGG, GGS, GSG, GGGS or GGGGS.

86. The conjugate of any one of claims 75-85, wherein Y comprises no more than 5 amino acids.

87. The conjugate of claim 86, wherein Y comprises an amino acid residue adjacent to J with polar uncharged side chain or positive charged side chain.

88. The conjugate of claim 87, wherein the amino acid residue adjacent to J is selected from the group consisting of Ala, Ser, Thr, Gln, Asn, Pro, His, and Arg.

89. The conjugate of any one of claims 75-88, wherein P comprises an amino acid sequence of anyone selected from the group consisting of GGGC, GGSC, GGGSC, GGGCA, GGGSCA, GGSCA, GGGSCSA, GGGSCYA, GGGSCQA, GGGSCPA, GGGSCHA, GGSCSA, GGSCYA, GGSCQA, GGSCPA, GGSCHA, GGGCSA, GGGCYA, GGGCQA, GGGCPA, GGGCHA, GGGGSCSA, GGGGSCYA, GGGGSCQA, GGGGSCPA, GGGGSCHA, GSGCSA, GSGCYA, GSGCQA, GSGCPA, and GSGCHA.

90. The conjugate of any one of claims 61-89, wherein L^{Z} is covalently conjugated with the thiol group of J by a structure of L^{Z} selected from the group consisting of

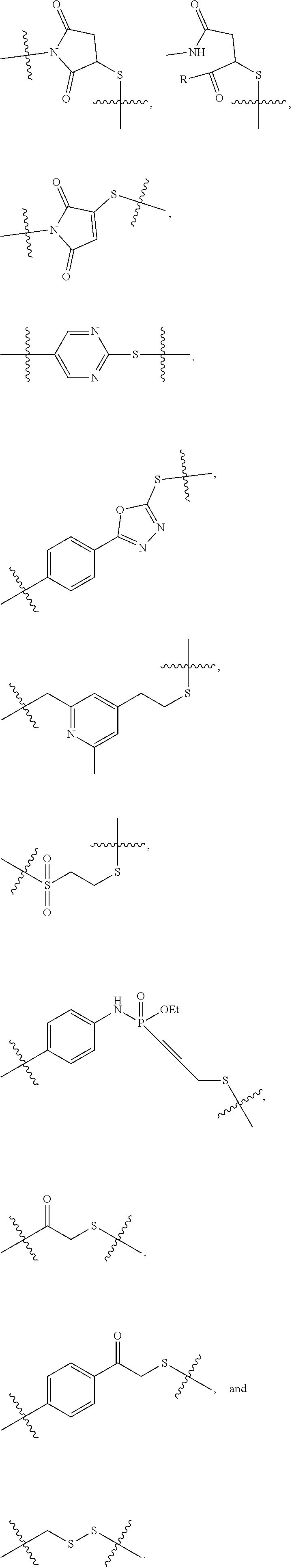

91. The conjugate of any one of claims 61-90, wherein the conjugate comprises a structure selected from any one of Table 3.

92. A method of preparing the conjugate of any one of claims 61-91, comprising:
(i) introducing into a host cell a nucleic acid encoding Z,
(ii) culturing the host cell under conditions that allow expression of Z;
(iii) isolating Z; and
(iv) conjugating Z with the compound of formula (I-X) via L^{Z(i)} of the compound: 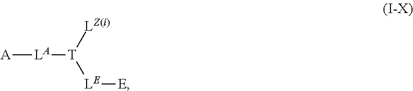
wherein:
(1) L^{Z(i)} is a linker comprising a thiol-reactive group or an amine-reactive group,
(2) T is a branching group which is at least trivalent,
(3) E is an effector moiety, wherein E is conjugated to T via a linker L^{E}, and
(4) A is a potency enhancing moiety, wherein A is conjugated to T via a linker L^{A}, wherein A, L^{A}, E, L^{E}, L^{Z}(i), T and Z are as defined in any one of claims 1-91.

93. The method of claim 92, wherein the host cell is a eukaryotic cell or a prokaryotic cell.

94. The method of claim 93, wherein the eukaryotic cell is a mammalian cell.

95. A pharmaceutical composition, comprising the conjugate of any one of claims 61-91 and a pharmaceutically acceptable excipient.

96. A kit, comprising the conjugate of any one of claims 61-91, and instructions for using the kit to diagnose a disease or disorder in a subject in need thereof.

97. A method of treating a disease or disorder in a subject in need thereof, comprising administering to the subject a pharmaceutical effective amount of the conjugate of any one of claims 61-91.

98. A method of inhibiting proliferative activity in a cell, comprising administering an effective amount of the conjugate of any one of claims 61-91.

99. A method of imaging a tissue in a subject by administering an imaging effective amount a pharmaceutical effective amount of the conjugate of any one of claims 61-91, to a subject in need thereof and applying an imaging technique to detect emitted gamma rays.

100. A method of diagnosing cancer in subject by administering a diagnostic effective amount a pharmaceutical effective amount of the conjugate of any one of claims 61-91 and applying an imaging technique to detect emitted gamma rays.


SEQUENCE LISTING (26 sequences)

SEQ ID NO: 1 - protein, 7 aa, synthetic construct
GGGSCSA

SEQ ID NO: 2 - protein, 7 aa, synthetic construct
GGGSCQA

SEQ ID NO: 3 - protein, 7 aa, synthetic construct
GGGSCPA

SEQ ID NO: 4 - protein, 7 aa, synthetic construct
GGGSCHA

SEQ ID NO: 5 - protein, 120 aa, synthetic construct
EVQLVESGGGLVQPGGSLRLSCAASGSFFRIVAMGWYRQAPGSQRELVATITRGGSTYYADSVKGRFTISRDNAKNTLYLQMNSLRAEDTAVYYCNVRVEVPFMQPNDYWGQGTLVTVSS

SEQ ID NO: 6 - protein, 8 aa, synthetic construct
GSFFRIVA

SEQ ID NO: 7 - protein, 7 aa, synthetic construct
ITRGGST

SEQ ID NO: 8 - protein, 14 aa, synthetic construct
NVRVEVPFMQPNDY

SEQ ID NO: 9 - protein, 127 aa, synthetic construct
EVQLVESGGGLVQPGGSLRLSCAASGSFFRIVAMGWYRQAPGSQRELVATITRGGSTYYADSVKGRFTISRDNAKNTLYLQMNSLRAEDTAVYYCNVRVEVPFMQPNDYWGQGTLVTVSSGGGSCSA

SEQ ID NO: 10 - protein, 124 aa, synthetic construct
EVQLVESGGGLVQPGGSLRLSCTGSARPFSNYGMGWFRQAPGNEREFVAAISGGGVYTDFADSVKGRFTISRDNSKNTLYLQMNSLRAEDTAVYYCARARTARVVSRRHEYDYWGQGTQVTVSS

SEQ ID NO: 11 - protein, 8 aa, synthetic construct
ARPFSNYG

SEQ ID NO: 12 - protein, 8 aa, synthetic construct
ISGGGVYT

SEQ ID NO: 13 - protein, 17 aa, synthetic construct
ARARTARVVSRRHEYDY

SEQ ID NO: 14 - protein, 131 aa, synthetic construct
EVQLVESGGGLVQPGGSLRLSCTGSARPFSNYGMGWFRQAPGNEREFVAAISGGGVYTDFADSVKGRFTISRDNSKNTLYLQMNSLRAEDTAVYYCARARTARVVSRRHEYDYWGQGTQVTVSSGGGSCSA

SEQ ID NO: 15 - protein, 131 aa, synthetic construct
EVQLVESGGGLVQPGGSLRLSCTGSARPFSNYGMGWFRQAPGNEREFVAAISGGGVYTDFADSVKGRFTISRDNSKNTLYLQMNSLRAEDTAVYYCARARTARVVSRRHEYDYWGQGTQVTVSSGGGSCQA

SEQ ID NO: 16 - protein, 131 aa, synthetic construct
EVQLVESGGGLVQPGGSLRLSCTGSARPFSNYGMGWFRQAPGNEREFVAAISGGGVYTDFADSVKGRFTISRDNSKNTLYLQMNSLRAEDTAVYYCARARTARVVSRRHEYDYWGQGTQVTVSSGGGSCPA

SEQ ID NO: 17 - protein, 131 aa, synthetic construct
EVQLVESGGGLVQPGGSLRLSCTGSARPFSNYGMGWFRQAPGNEREFVAAISGGGVYTDFADSVKGRFTISRDNSKNTLYLQMNSLRAEDTAVYYCARARTARVVSRRHEYDYWGQGTQVTVSSGGGSCHA

SEQ ID NO: 18 - protein, 119 aa, synthetic construct
QVQLLESGGGLVQPGGSLRLSCAASGFGFGNSLMSWVRRAPGKGLEWVSTIYPRGSFTDYADSVKGRFTISRDNSRNTLYLQMNSLRAEDTAVYYCATGWGRSSDYDPPGQGTLVTVSS

SEQ ID NO: 19 - protein, 8 aa, synthetic construct
GFGFGNSL

SEQ ID NO: 20 - protein, 8 aa, synthetic construct
IYPRGSFT

SEQ ID NO: 21 - protein, 12 aa, synthetic construct
ATGWGRSSDYDP

SEQ ID NO: 22 - protein, 126 aa, synthetic construct
QVQLLESGGGLVQPGGSLRLSCAASGFGFGNSLMSWVRRAPGKGLEWVSTIYPRGSFTDYADSVKGRFTISRDNSRNTLYLQMNSLRAEDTAVYYCATGWGRSSDYDPPGQGTLVTVSSGGGSCSA

SEQ ID NO: 23 - protein, 55 aa, synthetic construct
  VARIANT 5: note = X is selected from Phe or Tyr
  VARIANT 11: note = X is selected from Lys, Asn or Ala
  VARIANT 14: note = X is selected from Ser or Gly
  VARIANT 19: note = X is selected from Tyr or His
  VARIANT 20: note = X is selected from Gly or Asp
  VARIANT 23: note = X is selected from Lys or Met
  VARIANT 28: note = X is selected from Lys or Ala
  VARIANT 47: note = X is selected from Ser or Asn
  VARIANT 48: note = X is selected from Glu or Asp
  VARIANT 51: note = X is selected from null or Gly
  VARIANT 52: note = X is selected from null or Gly
  VARIANT 53: note = X is selected from null or Gly
  VARIANT 54: note = X is selected from Cys or Lys
  VARIANT 55: note = X is selected from null or Ala
AEAKXAKEKIXALXEIIWLPNLTXXQIXAFIAXLNDDPSQSSELLSEAKKLXXSQ

SEQ ID NO: 24 - protein, 55 aa, synthetic construct
AEAKFAKEKIKALSEIIWLPNLTYGQIKAFIAKLNDDPSQSSELLSEAKKLSESQ

SEQ ID NO: 25 - protein, 60 aa, synthetic construct
AEAKFAKEKIKALSEIIWLPNLTYGQIKAFIAKLNDDPSQSSELLSEAKKLSESQGGGCA

SEQ ID NO: 26 - protein, 61 aa, synthetic construct
AEAKYAKEMRNAYWEIALLPNLTNQQKRAFIRKLYDDPSQSSELLSEAKKLNDSQAPKVDC